(19) United States
(12) Patent Application Publication
(10) Pub. No.: US 2024/0301444 A1
(43) Pub. Date: Sep. 12, 2024

(54) GENOME EDITING COMPOSITIONS AND METHODS FOR TREATMENT OF CYSTIC FIBROSIS

(71) Applicant: PRIME MEDICINE, INC., Cambridge, MA (US)

(72) Inventors: Andrew V. ANZALONE, Cambridge, MA (US); Aaron Nakwon CHANG, Lexington, MA (US); Linghe XI, Cambridge, MA (US); Dewi HARJANTO, Brookline, MA (US)

(21) Appl. No.: 18/431,323

(22) Filed: Feb. 2, 2024

Related U.S. Application Data
(63) Continuation of application No. PCT/US2022/074642, filed on Aug. 5, 2022.
(60) Provisional application No. 63/229,827, filed on Aug. 5, 2021.

(51) Int. Cl. C12N 15/90; C12N 9/22; C12N 15/11

(52) U.S. Cl. CPC C12N 15/90; C12N 9/22; C12N 15/11; C12N 2310/20; C12N 2310/531

(57) ABSTRACT

Provided herein are compositions and methods of using prime editing systems comprising prime editors and prime editing guide RNAs for treatment of genetic disorders such as cystic fibrosis.

### CROSS-REFERENCE

This application is a continuation of International Application No. PCT/US22/74642, filed on Aug. 5, 2022, which claims the benefit of U.S. Provisional Application No. 63/229,827, filed Aug. 5, 2021, each of which applications are incorporated herein by reference in its entirety.

### SEQUENCE LISTING

The instant application contains a Sequence Listing which has been submitted electronically in XML file format and is hereby incorporated by reference in its entirety. Said XML copy, created on Aug. 4, 2022, is named 59761743601_SL.xml and is 19,326,690 bytes in size.

### BACKGROUND

Cystic fibrosis (CF) is one of the most common genetic disease, particularly in the Caucasian population. CF is caused by mutations in the cystic fibrosis transmembrane conductance regulator (CFTR/ABCC7) gene. Mutations in the CFTR gene results in defective biosynthesis, trafficking, and/or activity of the CFTR protein, causing severe damage to the lungs, pancreas, liver, intestines, sinuses, and limited ability to breath overtime. While technological advances have increased the life expectancy of certain CF patients, numerous mutations remain non-responsive to currently existing therapies, and there is still no effective cure for the disease.

This disclosure provides prime editing methods and compositions for correcting mutations in the CFTR gene associated with Cystic fibrosis.

### SUMMARY OF THE DISCLOSURE

Provided herein, in some embodiments, are methods and compositions for prime editing of alterations in a target sequence in a target gene, for example, a CFTR gene. The target CTFR gene may comprise double stranded DNA. As exemplified in FIG. 1, in some embodiment, the target gene, e.g., a CFTR gene, is edited by prime editing. In some embodiments, the prime editing described herein results in efficient correction of one or more pathogenic mutations in the CFTR gene, thereby treating cystic fibrosis in a subject.

Without wishing to be bound by any particular theory, the prime editing process may search specific targets and edit endogenous sequences in a target gene, e.g., the CFTR gene. As exemplified in FIG. 1, the spacer sequence of a PEgRNA recognizes and anneals with a search target sequence in a target strand of the target gene. A prime editing complex may generate a nick in the target gene on the edit strand which is the complementary strand of the target strand. The prime editing complex may then use a free 3′ end formed at the nick site of the edit strand to initiate DNA synthesis, where a primer binding site (PBS) of the PEgRNA complexes with the free 3′ end, and a single stranded DNA is synthesized using an editing template of the PEgRNA as a template. The editing template may comprise one or more nucleotide edits compared to the endogenous target CFTR gene sequence. Accordingly, the newly-synthesized single stranded DNA also comprises the nucleotide edit(s) encoded by the editing template. Through removal of an editing target sequence on the edit strand of the target gene and DNA repair, the intended nucleotide edit(s) included in the newly synthesized single stranded DNA are incorporated into the target CFTR gene.

One embodiment provides a prime editing guide RNA (PEgRNA) comprising: a spacer that is complementary to a search target sequence on a first strand of a CFTR gene, an editing template that comprises a region of complementarity to an editing target sequence on a second strand of the CFTR gene, and a gRNA core that associates with a prime editor comprising a DNA binding domain and a DNA polymerase domain, wherein the first strand and second strand are complementary to each other, and wherein the editing target sequence is in an exon selected from the group consisting of: exon 1, exon 2, exon 3, exon 4, exon 5, exon 6, exon 7, exon 8, exon 9, exon 10, exon 12, exon 13, exon 14, exon 15, exon 16, exon 17, exon 18, exon 19, exon 20, exon 21, exon 22, exon 23, exon 24, exon 25, exon 26, or exon 27 of the CFTR gene.

One embodiment provides a prime editing guide RNA (PEgRNA) comprising: a spacer that that is complementary to a search target sequence on a first strand of an CFTR gene, an editing template that comprises a region of complementarity to an editing target sequence on a second strand of the CFTR gene, and a gRNA core that associates with a prime editor comprising a DNA binding domain and a DNA polymerase domain, wherein first strand and second strand are complementary to each other, and wherein if the editing target sequence is in exon 11 then the editing target sequence does not comprise a CTT deletion at positions chr7:117559591-117559594 as compared to a wild type CFTR gene.

One embodiment provides a prime editing guide RNA (PEgRNA) comprising: a spacer that is complementary to a search target sequence on a first strand of an CFTR gene, an editing template that comprises a region of complementarity to an editing target sequence on a second strand of the CFTR gene, and a gRNA core that associates with a prime editor comprising a DNA binding domain and a DNA polymerase domain, wherein first strand and second strand are complementary to each other, and wherein the editing target sequence is between positions 117480095-117548823 and positions 117587739-117665564 of human chromosome 7.

One embodiment provides a prime editing guide RNA (PEgRNA) comprising: a spacer that is complementary to a search target sequence on a first strand of a CFTR gene, an editing template that comprises a region of complementarity to an editing target sequence on a second strand of the CFTR gene, and a gRNA core that associates with a prime editor comprising a DNA binding domain and a DNA polymerase domain, wherein first strand and second strand are complementary to each other, wherein the editing target sequence comprises a mutation associated with cystic fibrosis, and wherein the mutation is not a CTT deletion at positions chr7:117559591-117559594 as compared to a wild type CFTR gene.

In some embodiments, the PEgRNA comprises a primer binding site sequence (PBS) at least partially complementary to the spacer. In some embodiments, the gRNA core is between the spacer and the editing template. In some embodiments, the editing template comprises an intended nucleotide edit compared to the CFTR gene. In some embodiments, the PEgRNA guides the prime editor to incorporate the intended nucleotide edit into the CFTR gene when contacted with the CFTR gene. In some embodiments, the prime editor synthesizes a single stranded DNA encoded by the editing template, wherein the single stranded DNA replaces the editing target sequence and results in incorporation of the intended nucleotide edit into a region corresponding to the editing target in the CFTR gene. In some embodiments, the search target sequence is complementary to a protospacer sequence in the CFTR gene, and wherein the protospacer sequence is adjacent to a search target adjacent motif (PAM) in the CFTR gene. In some embodiments, the PEgRNA results in incorporation of the intended nucleotide edit in the PAM when contacted with the CFTR gene. In some embodiments, the PBS is about 2 to 20 base pairs in length. In some embodiments, the PBS is about 8 to 16 base pairs in length. In some embodiments, the editing template is about 4 to 30 base pairs in length. In some embodiments, the editing template is about 10 to 30 base pairs in length. In some embodiments, the PEgRNA results in incorporation of intended nucleotide edit about 0 to 27 base pairs downstream of the 5′ end of the PAM when contacted with the CFTR gene. In some embodiments, the intended nucleotide edit comprises a single nucleotide substitution compared to the region corresponding to the editing target in the CFTR gene. In some embodiments, the intended nucleotide edit comprise an insertion compared to the region corresponding to the editing target in the CFTR gene. In some embodiments, the intended nucleotide edit comprises a deletion compared to the region corresponding to the editing target in the CFTR gene. In some embodiments, the editing target sequence comprises a mutation associated with cystic fibrosis. In some embodiments, the editing template comprises a wild type CFTR gene sequence. In some embodiments, the PEgRNA results in correction of the mutation when contacted with the CFTR gene. In some embodiments, the editing target sequence is between positions 117509034 and 117509142 of human chromosome 7. In some embodiments, the editing target sequence is between positions 117530899 and 117531114 of human chromosome 7. In some embodiments, the editing target sequence is between positions 117540100 and 117540346 of human chromosome 7. In some embodiments, the editing target sequence is between positions 117548641 and 117548823 of human chromosome 7. In some embodiments, the editing target sequence is between positions 117559464 and 117559655 of human chromosome 7. In some embodiments, the editing target sequence is between positions 117587739 and 117587833 of human chromosome 7. In some embodiments, the editing target sequence is between positions 117591934 and 117592657 of human chromosome 7. In some embodiments, the editing target sequence is between positions 117614613 and 117614713 of human chromosome 7. In some embodiments, the editing target sequence is between positions 117627522 and 117627770 of human chromosome 7. In some embodiments, the editing target sequence is between positions 117642438 and 117642593 of human chromosome 7. In some embodiments, the editing target sequence is between positions 117652842 and 117652931 of human chromosome 7. In some embodiments, the editing target sequence comprises a mutation that encodes an amino acid substitution selected from a group consisting of G85E, R117H, R334W, R347P, R347H, A445E, G542*, S549N, G551D, R553*, K684fs, D1152H, R1158*, R1162*, K1177R, W1282*, and N1303K as compared to a wild type CFTR protein as set forth in SEQ ID NO: 19, wherein * refers to a nonsense mutation and fs refers to a frameshift mutation.

One embodiment provides a PEgRNA system comprising the PEgRNA according to this disclosure and further comprising a nick guide RNA (ngRNA), wherein the ngRNA comprises an ng spacer that is complementary to a second search target sequence in the CFTR gene. In some embodiments, the second search target sequence is on the second strand of the CFTR gene.

One embodiment provides a prime editing guide RNA (PEgRNA) comprising: a spacer comprising a sequence selected from the group consisting of SEQ Identifiers A330-A334, A343-A361, and A364-A380, an editing template that comprises a region of complementarity to an editing target sequence on an edit strand of a CFTR gene, and a gRNA core that associates with a prime editor comprising a DNA binding domain and a DNA polymerase domain, wherein the first strand and second strand are complementary to each other, and wherein the editing target sequence comprises a CTT deletion at positions chr7:117559591-117559594 as compared to a wild type CFTR gene. In some embodiments, the PEgRNA comprises a primer binding site sequence (PBS) that is at least partially complementary to the spacer. In some embodiments, the PBS comprises a sequence selected from the group consisting of SEQ Identifiers: B1377-B1403, B1413-B1520, and B1530-B1565. In some embodiments, the editing template comprises a sequence selected from the group consisting of SEQ Identifiers: C4592-C4964 and C4976-C5414.

One embodiment provides a prime editing guide RNA (PEgRNA) comprising: a spacer comprising a sequence selected from the group consisting of SEQ ID NOs. 678-682, 691-709, and 712-728, an editing template that comprises a region of complementarity to an editing target sequence on an edit strand of a CFTR gene, and a gRNA core that associates with a prime editor comprising a DNA binding domain and a DNA polymerase domain, wherein the first strand and second strand are complementary to each other, and wherein the editing target sequence comprises a CTT deletion at positions chr7:117559591-117559594 as compared to a wild type CFTR gene. In some embodiments, the PEgRNA comprises a primer binding site sequence (PBS) that is at least partially complementary to the spacer. In some embodiments, the PBS comprises a sequence selected from the group consisting of SEQ ID NOs: 2284-2304, 2312-2395, and 2403-2430. In some embodiments, the PEgRNA comprises a PBS comprising a sequence selected from the group consisting of sequences ATAATCCA, ATAATCCAG, CATAATCC, CATAATCCA, TGGTGCCA, TGGTGCCAG, TATTTTCT, TATTTTCTT, ATATTTTC, ATATTTTCT, GATATTTT, GATATTTTC, ATGAATAT, ATGAATATA, GCGTCATC, GCGTCATCA, TAATCCAG, TAATCCAGG, GTGTTTCC, GTGTTTCCT, TGAATATA, TGAATATAG, GATGAATA, GATGAATAT, ATGGTGCC, ATGGTGCCA, TGTTTCCT, TGTTTCCTA, TCCAGGAA, TCCAGGAAA, ATGATATT, ATGATATTT, TGATATTT, TGATATTTT, TCAAAGCA, TCAAAGCAT, GATGATAT, and GATGATATT. In some embodiments, the editing template comprises a sequence selected from the group consisting of SEQ ID NOs: 8618-8990 and 9002-9440.

One embodiment provides a prime editing complex comprising: (i) the PEgRNA of any one of the above embodiments or the PEgRNA system of any one of the above embodiments; and (ii) a prime editor comprising a DNA binding domain and a DNA polymerase domain. In some embodiments, the DNA binding domain is a CRISPR associated (Cas) protein domain. In some embodiments, the Cas protein domain has nickase activity. In some embodiments, the Cas protein domain is a Cas9. In some embodiments, the Cas9 comprises a mutation in an HNH domain. In some embodiments, the Cas9 comprises a H840A mutation in the HNH domain. In some embodiments, the Cas protein domain is a Cas12b. In some embodiments, the Cas protein domain is a Cas12a, Cas12b, Cas12c, Cas12d, Cas12e, Cas14a, Cas14b, Cas14c, Cas14d, Cas14e, Cas14f, Cas14g, Cas14h, Cas14u, or a CasΦ. In some embodiments, the DNA polymerase domain is a reverse transcriptase.

In some embodiments, the reverse transcriptase is a retrovirus reverse transcriptase. In some embodiments, the reverse transcriptase is a Moloney murine leukemia virus (M-MLV) reverse transcriptase. In some embodiments, the DNA polymerase and the programmable DNA binding domain are fused or linked to form a fusion protein. In some embodiments, the fusion protein comprises the sequence of any one of SEQ ID NOs: 15, 341, 342, or 343.

One embodiment provides a lipid nanoparticle (LNP) or ribonucleoprotein (RNP) comprising the prime editing complex of any one of the above embodiments, or a component thereof. A polynucleotide encoding the PEgRNA of any one of the above embodiments, the PEgRNA system of any one of the above embodiments, or the fusion protein of any one of the above embodiments. In some embodiments, the polynucleotide is a mRNA. In some embodiments, the polynucleotide is operably linked to a regulatory element. In some embodiments, the regulatory element is an inducible regulatory element.

One embodiment provides a vector comprising the polynucleotide of any one of the above embodiments. In some embodiments, the vector is an AAV vector. One embodiment provides an isolated cell comprising the PEgRNA of any one of the above embodiments, the PEgRNA system of any one of the above embodiments, the prime editing complex of any one of the above embodiments, the LNP or RNP of the above embodiment, the polynucleotide of any one of the above embodiment or the vector of any one of the above embodiments. In some embodiments, the cell is a human cell. In some embodiments, the cell is a fibroblast, an organoid cell, a basal cell, a club cell, an ionocyte, or an epithelial cell.

One embodiment provides a pharmaceutical composition comprising (i) the PEgRNA of any one of the above embodiments, the PEgRNA system of any one of the above embodiments, the prime editing complex of any one of the above embodiments, the LNP or RNP of above embodiment, the polynucleotide of any one of the above embodiments, the vector of any one of the above embodiments, or the cell of any one of the above embodiments; and (ii) a pharmaceutically acceptable carrier.

One embodiment provides a method for editing a CFTR gene, the method comprising contacting the CFTR gene with (i) the PEgRNA of any one of the above embodiments or the PEgRNA system of any one of the above embodiments and (ii) a prime editor comprising a DNA binding domain and a DNA polymerase domain, wherein the PEgRNA directs the prime editor to incorporate the intended nucleotide edit in the CFTR gene, thereby editing the CFTR gene.

One embodiment a method for editing an CFTR gene, the method comprising contacting the CFTR gene with the prime editing complex of any one of the above embodiments, wherein the PEgRNA directs the prime editor to incorporate the intended nucleotide edit in the CFTR gene, thereby editing the CFTR gene.

In some embodiments, the prime editor synthesizes a single stranded DNA encoded by the editing template, wherein the single stranded DNA replaces the editing target sequence and results in incorporation of the intended nucleotide edit into a region corresponding to the editing target in the CFTR gene. In some embodiments, wherein the CFTR gene is in a cell. In some embodiments, the cell is a mammalian cell. In some embodiments, the cell is a human cell. In some embodiments, the cell is a fibroblast, organoid cell, a basal cell, a club cell, an ionocyte, or an epithelial cell. In some embodiments, the cell is in a subject. In some embodiments, wherein the subject is a human. In some embodiments, the cell is from a subject having cystic fibrosis. In some embodiments, the method further comprises administering the cell to the subject after incorporation of the intended nucleotide edit.

One embodiment provides a cell generated by the method of any one of the above embodiments. One embodiment provides a population of cells generated by the method of any one of the above embodiments. One embodiment provides a method for treating cystic fibrosis in a subject in need thereof, the method comprising administering to the subject (i) the PEgRNA of any one of the above embodiments or the PEgRNA system of any one of the above embodiments and (ii) a prime editor comprising a DNA binding domain and a DNA polymerase domain, wherein the PEgRNA directs the prime editor to incorporate the intended nucleotide edit in the CFTR gene in the subject, thereby treating cystic fibrosis in the subject.

One embodiment provides a method for treating cystic fibrosis in a subject in need thereof, the method comprising administering to the subject the prime editing complex of any one of the above embodiments, the LNP or RNP of the above embodiment, or the pharmaceutical composition of claim 64, wherein the PEgRNA directs the prime editor to incorporate the intended nucleotide edit in the CFTR gene in the subject, thereby treating cystic fibrosis in the subject. In some embodiments, the subject is a human.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic of a prime editing guide RNA (PEgRNA) binding to a double stranded target DNA sequence.

FIG. 2 depicts a PEgRNA architectural overview in an exemplary schematic of PEgRNA designed for a prime editor.

FIG. 3 is a schematic showing the spacer and gRNA core part of an exemplary guide RNA, in two separate molecules. The rest of the PEgRNA structure is not shown.

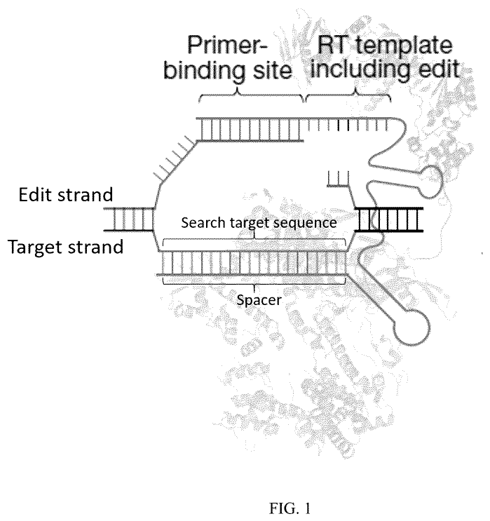

### DETAILED DESCRIPTION OF THE DISCLOSURE

Provided herein, in some embodiments, are compositions and methods to edit the target gene cystic fibrosis transmembrane conductance regulator (CFTR ABCC7) with prime editing. In certain embodiments, provided herein are compositions and methods for correction of mutations in the CFTR gene associated with cystic fibrosis. Compositions provided herein can comprise prime editors (PEs) that may use engineered guide polynucleotides, e.g., prime editing guide RNAs (PEgRNAs), that can direct PEs to specific DNA targets and can encode DNA edits on the target gene CFTR that serve a variety of functions, including direct correction of disease-causing mutations associated with cystic fibrosis.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

Unless otherwise specified, the words “comprising”, “comprise”, “comprises”, “having”, “have”, “has”, “including”, “includes”, “include”, “containing”, “contains” and “contain” are inclusive or open-ended and do not exclude additional, unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell).

In some embodiments, the cell is a human cell. A cell may be of or derived from different tissues, organs, and/or cell types. In some embodiments, the cell is a primary cell. As used herein, the term primary cell means a cell isolated from an organism, e.g., a mammal, which is grown in tissue culture (i.e., in vitro) for the first time before subdivision and transfer to a subculture. In some non-limiting examples, mammalian primary cells which can be transfected and further passaged include hepatocytes, fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells and precursors of these somatic cell types. In some embodiments, the cell is a primary cell. In some embodiments, the cell is a human primary cell. In some embodiments, the cell is a pluripotent stem cell. In some embodiments, the cell is an induced human pluripotent stem cell (iPSC). In some embodiments, the cell is a lung progenitor cell. In some embodiments, the cell is a basal cell. In some embodiments, the cell is a basal cell from the respiratory epithelium, e.g., from the bronchioles or alveoli of the lung. In some embodiments, the cell is a club cell. In some embodiments, the cell is a ciliated cell. In some embodiments, the cell is an ionocyte. In some embodiments, the cell is a human basal cell. In some embodiments, the cell is a human basal cell from the respiratory epithelium, e.g., from the bronchioles or alveoli of the lung. In some embodiments, the cell is a human club cell. In some embodiments, the cell is a human ciliated cell. In some embodiments, the cell is a human ionocyte. In some embodiments, the cell is a bronchioalveolar stem cell. In some embodiments, the cell is a human bronchioalveolar stem cell. In some embodiments, the cell is a human lung progenitor cell. In some embodiments, the cell is an epithelial cell. In some embodiments, the cell is an airway epithelial cell. In some embodiments, the cell is a human airway epithelial cell. In some embodiments, the cell is a bronchial epithelial cell. In some embodiments, the cell is a human bronchial epithelial cell. In some embodiments, the cell is a human bronchial epithelial cell induced from iPSC. In some embodiments, the cell is a pancreatic epithelial cell. In some embodiments, the cell is a pancreatic ductal epithelia cell. In some embodiments, the cell is a pancreatic acinar cell. In some embodiments, the cell is a human pancreatic epithelial cell. In some embodiments, the cell is a human pancreatic ductal epithelia cell. In some embodiments, the cell is a human pancreatic acinar cell. In some embodiments, the cell is a hepatocyte. In some embodiments, the cell is a human hepatocyte. In some embodiments, the cell is a kidney epithelial cell. In some embodiments, the cell is an intestine epithelial cell. In some embodiments, the cell is a human kidney epithelial cell. In some embodiments, the cell is a human intestine epithelial cell. In some embodiments, the cell is a fibroblast. In some embodiments, the cell is a human fibroblast. In some embodiments, the cell is a lymphocyte. In some embodiments, the cell is a human lymphocyte. In some embodiments, the cell is a tuft cell. In some embodiments, the cell is a neuroendocrine cell. In some embodiments, the cell is a goblet cell. In some embodiments, the cell is a reproductive tissue epithelial cell. In some embodiments, the cell is a sperm canal epithelial cell. In some embodiments, the cell is a human tuft cell. In some embodiments, the cell is a human neuroendocrine cell. In some embodiments, the cell is a human goblet cell. In some embodiments, the cell is a human reproductive tissue epithelial cell. In some embodiments, the cell is a human sperm canal epithelial cell. In some embodiments, the cell is derived from a human subject. In some embodiments, the cell is derived from a human subject having a disease or condition associated with a target gene, e.g., CFTR. In some embodiments, the cell is a part of an organoid, e.g., an intestinal organoid. In some embodiments, the cell is part of a human organoid.

In some embodiments, the cell comprises a prime editor, a PEgRNA, or a prime editing composition disclosed herein. In some embodiments, the cell further comprises an ngRNA. In some embodiments, the cell is from a human subject. In some embodiments, the human subject has a disease or condition associated with a mutation to be corrected by prime editing, for example, cystic fibrosis. In some embodiments, the cell is from a human subject, and comprises a prime editor, a PEgRNA, or a prime editing composition for correction of the mutation. In some embodiments, the cell is from the human subject and the mutation has been edited or corrected by prime editing.

The term “substantially” as used herein may refer to a value approaching 100% of a given value. In some embodiments, the term may refer to an amount that may be at least about 70%, 80%, 90%, 91%, 92%, 93%, 94%, 95%, 96%, 97%, 98%, 99%, 99.9%, or 99.99% of a total amount. In some embodiments, the term may refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein may be a full-length protein (e.g., a fully processed protein having certain biological function). In some embodiments, a protein may be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor may be a fusion protein comprising a Cas9 protein domain of S. pyogenes and a reverse transcriptase protein domain of a Moloney murine leukemia virus (M-MLV). A protein that comprises amino acid sequences from different origins or naturally occurring proteins may be referred to as a fusion, or chimeric protein.

In some embodiments, a protein comprises a functional variant or functional fragment of a full-length wild type protein. A “functional fragment” or “functional portion”, as used herein, refers to any portion of a reference protein (e.g., a wild type protein) that encompasses less than the entire amino acid sequence of the reference protein while retaining one or more of the functions, e.g., catalytic or binding functions. For example, a functional fragment of a reverse transcriptase may encompass less than the entire amino acid sequence of a wild type reverse transcriptase, but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild type Cas9 but retains its DNA binding ability and lacks its nuclease activity partially or completely.

A “functional variant” or “functional mutant”, as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild type reverse transcriptase, but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional fragment of a Cas9 may comprise one or more amino acid substitutions in a nuclease domain, e.g., an H840A amino acid substitution, compared to the amino acid sequence of a wild type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

In some embodiments, a protein or polypeptides includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid and a corresponding reference amino acid sequence or a polynucleotide sequence and a corresponding reference polynucleotide sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, a primer binding site, or protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16: 276-277), and the GGSEARCH program https://fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). In some embodiments, alignment between a query sequence and a reference sequence is performed with Needleman-Wunsch alignment with Gap Costs set to Existence: 11 Extension: 1 where percent identity is calculated by dividing the number of identities by the length of the alignment, as further described in Altschul et al. (“Gapped BLAST and PSI-BLAST: a new generation of protein database search programs”, Nucleic Acids Res. 25:3389-3402, 1997) and Altschul et al, (“Protein database searches using compositionally adjusted substitution matrices”, FEBS J. 272:5101-5109, 2005).

A skilled person understands that amino acid (or nucleotide) positions may be determined in homologous sequences based on alignment, for example, “H840” in a reference Cas9 sequence may correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G).

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to base pair with each other. Complementary polynucleotides may base pair via hydrogen bonding, which may be Watson Crick, Hoogsteen or reversed Hoogsteen hydrogen bonding. For example, an adenine on one polynucleotide molecule will base pair to a thymine or an uracil on a second polynucleotide molecule and a cytosine on one polynucleotide molecule will base pair to a guanine on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. “Substantial complementary” can also refer to a 100% complementarity over a portion or a region of two polynucleotide molecules. In some embodiments, the portion or the region of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, translated into peptides, polypeptides, or proteins. If the polynucleotide is derived from genomic DNA, expression may include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript, that is encoded by the gene after transcription the gene. In some embodiments, expression of a polynucleotide, e.g., an mRNA, is determined by the amount of the protein encoded by the mRNA after translation of the mRNA. In some embodiments, expression of a polynucleotide, e.g., a mRNA or coding RNA, is determined by the amount of a functional form of the protein encoded by the polypeptide after translation of the polynucleotide.

The term “sequencing” as used herein, may comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, or any combination thereof.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or a reference nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” and its grammatical equivalents as used herein may refer to a human or a non-human. A subject may be a mammal. A human subject may be male or female. A human subject may be of any age. A subject may be a human embryo. A human subject may be a newborn, an infant, a child, an adolescent, or an adult. A human subject may be in need of treatment for a genetic disease or disorder.

The terms “treatment” or “treating” and their grammatical equivalents may refer to the medical management of a subject with an intent to cure, ameliorate, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates, but does not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g., a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective amount” or “therapeutically effective amount” refers to a quantity of a composition, for example a prime editing composition comprising a construct, that can be sufficient to result in a desired activity upon introduction into a subject as disclosed herein. An effective amount of the prime editing compositions can be provided to the target gene or cell, whether the cell is ex vivo or in vivo. An effective amount can be the amount to induce, for example, at least about a 2-fold change (increase or decrease) or more in the amount of target nucleic acid modulation (e.g., expression of a target CFTR gene to produce functional CFTR protein) observed relative to a negative control. An effective amount or dose can induce, for example, about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in target gene modulation (e.g., expression of a target CFTR gene to produce functional CFTR protein). The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro or in vivo).

In some embodiments, an effective amount can be an amount to induce, when administered to a population of cells, a certain percentage of the population of cells to have a correction of a mutation. For example, in some embodiments, an effective amount can be the amount to induce, when administered to or introduced to a population of cells, installation of one or more intended nucleotide edits that correct a mutation in the target CFTR gene, in at least about 1%, 2%, 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the population of cells.

Cystic fibrosis or “CF” refers to a genetic disorder caused by pathogenic mutations or variants in the CFTR gene that decrease expression or function of the CFTR protein. The CFTR protein is a member of the C subfamily of ABC transporters. In some embodiments, the CFTR protein functions in fluid transport and surface hydration across epithelial cells. In some embodiments, CF is associated with a single mutation in the CFTR gene in a subject. In some embodiments, CF is associated with multiple mutations in the CFTR gene in a subject. In some embodiments, CF associates with a different set of one or more mutations in the CFTR gene in a group or a population of subjects. In some embodiments, CF is inherited in a recessive manner. In some embodiments, CF is associated with one or more mutations in both copies of the CFTR gene. In some embodiments, mutations in the CFTR gene result in loss of CFTR expression and/or activity and cause inflammation and infection within the lungs and intestines and other severe symptoms. In some embodiments, the CFTR gene or the CFTR protein is also referred to as the ABCC7 gene or the ABCC7 protein. In the human genome, the CFTR gene is located on q31.2 of chromosome 7 and contains 26 introns and 27 exons (chr7: 117480025-117668665 (GRCh38/hg38)).

### Prime Editing

The term “prime editing” refers to programmable editing of a target DNA using a prime editor complexed with a PEgRNA to incorporate an intended nucleotide edit (also referred to herein as a nucleotide change) into the target DNA through target-primed DNA synthesis. A target gene of prime editing may comprise a double stranded DNA molecule having two complementary strands: a first strand that may be referred to as a “target strand” or a “non-edit strand”, and a second strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally protospacer adjacent motif (PAM) sequence. In prime editing using a Cas-protein-based prime editor, a PAM sequence refers to a short DNA sequence immediately adjacent to the protospacer sequence on the PAM strand of the target gene. A PAM sequence may be specifically recognized by a programmable DNA binding protein, e.g., a Cas nickase or a Cas nuclease. In some embodiments, a specific PAM is characteristic of a specific programmable DNA binding protein, e.g., a Cas nickase or a Cas nuclease. A protospacer sequence refers to a specific sequence in the PAM strand of the target gene that is complementary to the search target sequence. In a PEgRNA, a spacer sequence may have a substantially identical sequence as the protospacer sequence on the edit strand of a target gene, except that the spacer sequence may comprise Uracil (U) and the protospacer sequence may comprise Thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is upstream of a PAM sequence recognized by a Cas9 nickase, wherein the Cas9 nickase comprises a nuclease active RuvC domain and a nuclease inactive HNH domain. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 base pairs upstream of the PAM sequence, and the PAM sequence is recognized by a Cas9 nickase, wherein the Cas9 nickase that comprises a nuclease active RuvC domain and a nuclease inactive HNH domain. In some embodiments, the nick site is 2 nucleotides upstream of the PAM sequence, and the PAM sequence is recognized by a S. thermophilus Cas9 nickase that comprises a nuclease active RuvC domain and a nuclease inactive HNH domain.

A “primer binding site” (also referred to as PBS or primer binding site sequence) is a single-stranded portion of the PEgRNA that comprises a region of complementarity to the PAM strand (i.e., the non-target strand or the edit strand). The PBS is complementary or substantially complementary to a sequence on the PAM strand of the double stranded target DNA that is immediately upstream of the nick site. In some embodiments, in the process of prime editing, the PEgRNA complexes with and directs a prime editor to bind the search target sequence on the target strand of the double stranded target DNA and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

An “editing template” of a PEgRNA is a single-stranded portion of the PEgRNA that is 5′ of the PBS and comprises a region of complementarity to the PAM strand (i.e., the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other. In some embodiments, the single stranded portion of the PEgRNA comprising both the PBS and the editing template is complementary or substantially complementary to an endogenous sequence on the PAM strand (i.e., the non-target strand or the edit strand) of the double stranded target DNA except for one or more non-complementary nucleotides at the intended nucleotide edit position(s). As used herein, regardless of relative 5′-3′ positioning in other context, the relative positions as between the PBS and the editing template, and the relative positions as among elements of a PEgRNA, are determined by the 5′ to 3′ order of the PEgRNA regardless of the position of sequences in the double stranded target DNA that may have complementarity or identity to elements of the PEgRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits. In some embodiments, the editing template may encode the wild-type or non-disease associated gene sequence (or its complement if the edit strand is the antisense strand of a gene). In some embodiments, the editing template may encode the wild-type or non-disease associated protein, but contain one or more synonymous mutations relative to the wild-type or non-disease associated protein coding region. Such synonymous mutations may include, for example, mutations that decrease the ability of a PEgRNA to rebind to the same target sequence once the desired edit is installed in the genome (e.g., synonymous mutations that silence the endogenous PAM sequence or that edit the endogenous protospacer).

In some embodiments, a PEgRNA complexes with and directs a prime editor to bind to the search target sequence of the target gene at the nick site. In some embodiments, the bound prime editor generates a nick on the edit strand (PAM strand) of the target gene. In some embodiments, a primer binding site (PBS) of the PEgRNA anneals with a free 3′ end formed at the nick site, and the prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to the endogenous target gene sequence. Accordingly, in some embodiments, the editing template of a PEgRNA is complementary to a sequence in the edit strand except for one or more mismatches at the intended nucleotide edit positions in the editing template. The endogenous, e.g., genomic, sequence that is partially complementary to the editing template may be referred to as an “editing target sequence”. Accordingly, in some embodiments, the newly synthesized single stranded DNA has identity or substantial identity to a sequence in the editing target sequence, except for one or more insertions, deletions, or substitutions at the intended nucleotide edit positions. In some embodiments, the editing template comprises at least 4 contiguous nucleotides of complementarity with the edit strand wherein the at least 4 nucleotides contiguous are located upstream of the 5′ most edit in the editing template.

In some embodiments, the newly synthesized single-stranded DNA equilibrates with the editing target on the edit strand of the target gene for pairing with the target strand of the target gene. In some embodiments, the editing target sequence of the target gene is excised by a flap endonuclease (FEN), for example, FEN1. In some embodiments, the FEN is an endogenous FEN, for example, in a cell comprising the target gene. In some embodiments, the FEN is provided as part of the prime editor, either linked to other components of the prime editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the target gene. In some embodiments, the newly synthesized single-stranded DNA comprising the nucleotide edit is paired in the heteroduplex with the target strand of the target DNA that does not comprise the nucleotide edit, thereby creating a mismatch between the two otherwise complementary strands. In some embodiments, the mismatch is recognized by DNA repair machinery, e.g., an endogenous DNA repair machinery. In some embodiments, through DNA repair, the intended nucleotide edit is incorporated into the target gene.

### Prime Editor

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide (DNA binding domain) and a reverse transcriptase polypeptide (DNA polymerase) that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

### Prime Editor Nucleotide Polymerase Domain

In some embodiments, a prime editor comprises a nucleotide polymerase domain, e.g., a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the prime editor comprises a DNA-dependent DNA polymerase. For example, a prime editor having a DNA-dependent DNA polymerase can synthesize a new single stranded DNA using a PEgRNA editing template that comprises a DNA sequence as a template. In such cases, the PEgRNA is a chimeric or hybrid PEgRNA, and comprising an extension arm comprising a DNA strand. The chimeric or hybrid PEgRNA may comprise an RNA portion (including the spacer and the gRNA core) and a DNA portion (the extension arm comprising the editing template that includes a strand of DNA).

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol IV family DNA polymerase. In some embodiments, the DNA polymerase is a E. coli Pol IV DNA polymerase. In some embodiments, the DNA polymerase comprises a eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLDI DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a Rev1 DNA polymerase. In some embodiments, the DNA polymerase is a human Rev1 DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, a prime editor comprises an RNA-dependent DNA polymerase domain, for example, a reverse transcriptase (RT). A RT or an RT domain may be a wild type RT domain, a full-length RT domain, or may be a functional mutant, a functional variant, or a functional fragment thereof. An RT or an RT domain of a prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, a prime editor comprises a virus RT, for example, a retrovirus RT. Non-limiting examples of virus RT include Moloney murine leukemia virus (M-MLV MMLVRT or M-MLV RT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild-type M-MLV RT, a functional mutant, a functional variant, or a functional fragment thereof. In some embodiments, the prime editor comprises a reference M-MLV RT, a functional mutant, a functional variant, or a functional fragment thereof. In some embodiments, the RT domain or a RT is a M-MLV RT (e.g., wild-type M-MLV RT, a functional mutant, a functional variant, or a functional fragment thereof). In some embodiments, the RT domain or a RT is a M-MLV RT (e.g., a reference M-MLV RT, a functional mutant, a functional variant, or a functional fragment thereof). In some embodiments, a M-MLV RT, e.g., reference M-MLV RT, comprises an amino acid sequence as set forth in any one of SEQ ID NO: 1.

In some embodiments, a reference M-MLV RT is a wild-type M-MLV RT. An exemplary amino acid sequence of a reference M-MLV RT is provided in SEQ ID NO: 2.

In some embodiments, the prime editor comprises a wild type M-MLV RT. An exemplary amino acid sequence of a wild type M-MLV RT is provided in SEQ ID NO: 2.

(SEQ ID NO: 2)
TLNIEDEHRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGTAGFCRLWIPGFAEMAAPLYPLTKTGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGLLTSEGKEIKNKDEILALLKALFLPKRLSIIHCPGHQKGHSAEARGNR
MADQAARKAAITETPDTSTLLIENSSP.

In some embodiments, the prime editor comprises a reference M-MLV RT. An exemplary amino acid sequence of a reference M-MLV RT is provided in SEQ ID NO: 1.

Exemplary wild type Moloney murine leukemia virus reverse transcriptase:

(SEQ ID NO: 1)
TLNIEDEYRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGTAGFCRLWIPGFAEMAAPLYPLTKTGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGLLTSEGKEIKNKDEILALLKALFLPKRLSIIHCPGHQKGHSAEARGNR
MADQAARKAAITETPDTSTLLIENSSP.

In some embodiments, the prime editor comprises a M-MLV RT comprising one or more of amino acid substitutions P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to the reference M-MLV RT as set forth in SEQ ID NO: 1, where X is any amino acid other than the wild type amino acid. In some embodiments, the prime editor comprises a M-MLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to the reference M-MLV RT as set forth in SEQ ID NO: 1. In some embodiments, the prime editor comprises a M-MLV RT comprising one or more amino acid substitutions D200N, T330P, L603W, T306K, and W313F as compared to the reference M-MLV RT as set forth in SEQ ID NO: 1. In some embodiments, the prime editor comprises a M-MLV RT comprising amino acid substitutions D200N, T330P, L603W, T306K, and W313F as compared to the reference M-MMLV RT as set forth in SEQ ID NO: 1. In some embodiments, a prime editor comprising the D200N, T330P, L603W, T306K, and W313F as compared to a reference M-MLV RT as set forth in SEQ ID NO: 1. In some embodiments, the prime editor comprises a M-MLV RT comprising one or more of amino acid substitutions D200N, T330P, L603W, T306K, and W313F as compared to a wild type M-MMLV RT as set forth in SEQ ID NO: 2. In some embodiments, a prime editor may comprise amino acid substitutions D200N, T330P, L603W, T306K, and W313F as compared to a reference M-MLV RT as set forth in SEQ ID NO: 1. In some embodiments, the prime editor comprises a M-MLV RT that comprises an amino acid sequence that is at least 70%, at least 75%, at least 80%, at least 85%, at least 90%, at least 95%, at least 96%, at least 97%, at least 98%, at least 99%, at least 99.5%, or at least 99.9% identical to an amino acid sequence set forth in any one of SEQ ID NOs: 1-3. In some embodiments, the prime editor comprises a M-MLV RT that comprises an amino acid sequence that is selected from the group consisting of SEQ ID NOs: 1, 2 and 3 or a variant or fragment thereof. In some embodiments, the prime editor comprises a M-MLV RT that comprises an amino acid sequence set forth in SEQ ID NO: 3.

(SEQ ID NO: 3)
TLNIEDEYRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFN
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGKAGFCRLFIPGFAEMAAPLYPLTKPGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGWLTSEGKEIKNKDEILALLKALFLPKRLSIIHCPGHQKGHSAEARGNR
MADQAARKAAITETPDTSTLLIENSSP.

A prime editing system comprising a PEgRNA (or one or more polynucleotide encoding the PEgRNA) and a prime editor protein (or one or more polynucleotides encoding the prime editor), may be referred to as a PE2 prime editing system and the corresponding editing approach referred to as PE2 approach or PE2 strategy. A PE2 system does not contain a ngRNA. A prime editing system comprising a PEgRNA (or one or more polynucleotide encoding the PEgRNA), a prime editor protein (or one or more polynucleotides encoding the prime editor), and a ngRNA (or one or more polynucleotides encoding the ngRNA) may be referred to as a “PE3” prime editing system. In some embodiments, an ngRNA spacer sequence is complementary to a portion of the edit strand that includes the intended nucleotide edit and may hybridize with the edit strand only after the edit has been incorporated on the edit strand. Such ngRNA may be referred to a “PE3b” ngRNA, and the prime editing system a PE3b prime editing system.

In some embodiments, a prime editor comprises a eukaryotic RT, for example, a yeast, drosophila, rodent, or primate RT. In some embodiments, the prime editor comprises a Group II intron RT, for example, a Geobacillus stearothermophilus Group II Intron (GsI-IIC) RT or a Eubacterium rectale group II intron (Eu.re.I2) RT. In some embodiments, the prime editor comprises a retron RT.

In some embodiments, the DNA-binding domain of a prime editor is a programmable DNA binding domain. In some embodiments, a prime editor comprises a DNA binding domain that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 298-325. In some embodiments, the DNA binding domain comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions, substitutions and/or insertions compared to any one of the amino acid sequences set forth in SEQ ID NOs: 298-325. In some embodiments, the DNA binding domain of a prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a target gene. In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprises a nuclease activity. In some embodiments, the DNA-binding domain of a prime editor comprises an endonuclease domain having single strand DNA cleavage activity. For example, the endonuclease domain may comprise a FokI nuclease domain. In some embodiments, the DNA-binding domain of a prime editor comprises a nuclease having full nuclease activity. In some embodiments, the DNA-binding domain of a prime editor comprises a nuclease having modified or reduced nuclease activity as compared to a wild type endonuclease domain. For example, the endonuclease domain may comprise one or more amino acid substitutions as compared to a wild type endonuclease domain. In some embodiments, the DNA-binding domain of a prime editor has nickase activity. In some embodiments, the DNA-binding domain of a prime editor comprises a Cas protein domain that is a nickase. In some embodiments, compared to a wild type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas9, Cas12a (Cpf1), Cas12e (CasX), Cas12d (CasY), Cas12b1 (C2c1), Cas12b2, Cas12c (C2c3), C2c4, C2c8, C2c5, C2c10, C2c9, Cas14a, Cas14b, Cas14c, Cas14d, Cas14e, Cas14f, Cas14g, Cas14h, Cas14u, Cns2, Cas Φ, and homologs, functional fragments, or modified versions thereof. A Cas protein can be a chimeric Cas protein that is fused to other proteins or polypeptides. A Cas protein can be a chimera of various Cas proteins, for example, comprising domains of Cas proteins from different organisms.

A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis.

Non-limiting examples of suitable organism include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some embodiments, the organism is Streptococcus pyogenes (S. pyogenes). In some embodiments, the organism is Staphylococcus aureus (S. aureus). In some embodiments, the organism is Streptococcus thermophilus (S. thermophilus). In some embodiments, the organism is Staphylococcus lugdunensis (S. lugdunensis).

In some embodiments, a Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

In some embodiments, a Cas protein, e.g., Cas9, can be a wild type or a modified form of a Cas protein. In some embodiments, a Cas protein, e.g., Cas9, can be a nuclease active variant, nuclease inactive variant, a nickase, or a functional variant or a functional fragment of a wild type Cas protein. In some embodiments, a Cas protein, e.g., Cas9, can be a wild type or a modified form of a Cas protein. A Cas protein, e.g., Cas9, can be a nuclease active variant, nuclease inactive variant, a nickase, or a functional variant or functional fragment of a wild type Cas protein. In some embodiments, a Cas protein, e.g., Cas9, can comprise an amino acid change such as a deletion, insertion, substitution, fusion, chimera, or any combination thereof relative to a corresponding wild-type version of the Cas protein. In some embodiments, a Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a PEgRNA.

In some embodiments, a prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand or the non-edit strand of the target gene but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain e.g., an amino acid substitution that reduces or abolishes nuclease activity of the RuvC domain. In some embodiments, the Cas9 nickase comprises a D10X amino acid substitution compared to a wild type S. pyogenes Cas9, wherein X is any amino acid other than D. In some embodiments, a prime editor comprises a Cas9 nickase having an amino acid substitution in the HNH domain e.g., an amino acid substitution that reduces or abolishes nuclease activity of the HNH domain. In some embodiments, the Cas9 nickase comprises a H840X amino acid substitution compared to a wild type S. pyogenes Cas9, wherein X is any amino acid other than H.

In some embodiments, a prime editor comprises a Cas protein that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some embodiments, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild type Cas9 protein from any organism or a homolog, ortholog, or paralog from any organisms; any functional mutants or functional variants thereof; or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild type reference Cas9 protein (e.g., Cas9 from S. pyogenes). In some embodiments, the Cas9 comprises an amino acid change such as a deletion, insertion, substitution, fusion, chimera, or any combination thereof as compared to a wild type reference Cas9 protein. Exemplary Cas sequences are provided in Table 9 below.

In some embodiments, a Cas9 protein comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 298-325. In some embodiments, a Cas9 protein is a Cas9 nickase that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 299, 300, 302, 303, 305, 306, 308, 309, 311, 312, 314, 315, 317, 318, 320, 321, 323, 324, or 325. In some embodiments, a Cas9 protein comprises an amino acid sequence that is selected from the group consisting of SEQ ID NOs: 298-325. In some embodiments, a prime editor comprises a Cas9 protein that comprises an amino acid sequence that lacks a N-terminus methionine relative to an amino acid sequence set forth in any one of SEQ ID NOs: 298, 301, 304, 307, 310, 313, 316, 319, or 322. In some embodiments, the prime editing compositions or prime editing systems disclosed herein comprises a polynucleotide (e.g., a DNA, or an RNA, e.g., an mRNA) that encodes a Cas9 protein that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 298-325.

In some embodiments, a Cas9 protein comprises a Cas9 protein from Streptococcus pyogenes (Sp), e.g., as according to NC_002737.2:854751-858857 or the protein encoded by UniProt Q99ZW2, e.g., as according to SEQ ID NO: 298. In some embodiments, a prime editor comprises a Cas9 protein (e.g., a SpCas9) as according to any one of the sequences set forth in SEQ ID NOs: 298-300, or 325 or a variant thereof. In some embodiments, the Cas9 protein is a SpCas9. In some embodiments, a SpCas9 can be a wild type SpCas9, a SpCas9 variant, or a nickase SpCas9. In some embodiments, the SpCas9 lacks the N-terminus methionine relative to a corresponding SpCas9 (e.g., a wild type SpCas9, a SpCas9 variant or a nickase SpCas9). In some embodiments, a prime editor comprises a Cas9 protein, having an amino acid sequence as according to SEQ ID NO: 298, not including the N-terminus methionine. In some embodiments, a wild type SpCas9 comprises an amino acid sequence set forth in SEQ ID NO: 298. In some embodiments, a prime editor comprises a Cas9 protein comprising one or more mutations (e.g., amino acid substitutions, insertions and/or deletions) relative to a corresponding wild type Cas9 protein (e.g., a wild type SpCas9). In some embodiments, the Cas9 protein comprising one or more mutations relative to a wild type Cas9 (e.g., a wild type SpCas9) protein comprises an amino acid sequence set forth in SEQ ID NO: 299, SEQ ID NO: 300 or SEQ ID NO: 325. Exemplary Streptococcus pyogenes Cas9 (SpCas9) amino acid sequence useful in the prime editors disclosed herein are provided below in SEQ ID NOs: 298-300, and 325.

In some embodiments, a prime editor comprises a Cas9 protein (e.g., a SluCas9) as according to any one of the SEQ ID NOS: 301-303 or a variant thereof. In some embodiments, a prime editor comprises a Cas9 protein from Staphylococcus lugdunensis (SluCas9) e.g., as according to any one of the SEQ ID NOs: 301-303, or a variant thereof. In some embodiments, the Cas9 protein is a SluCas9. In some embodiments, a SluCas9 can be a wild type SluCas9, a SluCas9 variant, or a nickase SluCas9. In some embodiments, the SluCas9 lacks the N-terminus methionine relative to a corresponding SluCas9 (e.g., a wild type SluCas9, a SluCas9 variant or a nickase SluCas9). In some embodiments, a prime editor comprises a Cas9 protein, having an amino acid sequence as according to SEQ ID NO: 301, not including the N-terminus methionine. In some embodiments, a wild type SluCas9 comprises an amino acid sequence set forth in SEQ ID NO: 301. In some embodiments, a prime editor comprises a Cas9 protein comprising one or more mutations (e.g., amino acid substitutions, insertions and/or deletions) relative to a corresponding wild type Cas9 protein (e.g., a wild type SluCas9). In some embodiments, the Cas9 protein comprising one or mutations relative to a wild type Cas9 protein comprises an amino acid sequence set forth in SEQ ID NO: 302 or SEQ ID NO: 303. Exemplary Staphylococcus lugdunensis Cas9 (SluCas9) amino acid sequence useful in the prime editors disclosed herein are provided below in SEQ ID NOs: 301-303.

In some embodiments, a prime editor comprises a Cas9 protein from Staphylococcus aureus (SaCas9) e.g., as according to any of the SEQ ID NOS: 304-306, or a variant thereof. In some embodiments, a prime editor comprises a Cas9 protein from Staphylococcus aureus (SaCas9) e.g., as according to any one of the SEQ ID NOS: 304-306, or a variant thereof. In some embodiments, the Cas9 protein is a SaCas9. In some embodiments, a SaCas9 can be a wild type SaCas9, a SaCas9 variant, or a nickase SaCas9. In some embodiments, the SaCas9 lacks the N-terminus methionine relative to a corresponding SaCas9 (e.g., a wild type SaCas9, a SaCas9 variant or a nickase SaCas9). In some embodiments, a prime editor comprises a Cas9 protein, having an amino acid sequence as according to SEQ ID NO: 304, not including the N-terminus methionine. In some embodiments, a wild type SaCas9 comprises an amino acid sequence set forth in SEQ ID NO: 304. In some embodiments, a prime editor comprises a Cas9 protein comprising one or more mutations (e.g., amino acid substitutions, insertions and/or deletions relative to a corresponding wild type Cas9 protein (e.g., a wild type SaCas9). In some embodiments, the Cas9 protein comprising one or more mutations relative to a wild type Cas9 protein comprises an amino acid sequence set forth in SEQ ID NO: 305 or SEQ ID NO: 306. Exemplary Staphylococcus aureus Cas9 (SaCas9) amino acid sequence useful in the prime editors disclosed herein are provided below in SEQ ID NOs: 304-306.

In some embodiments, a prime editor comprises a Cas protein, e.g., a Cas9 variant, comprising modifications that allow altered PAM recognition. Exemplary Cas9 protein amino acid sequence (e.g., Cas9 variant with altered PAM recognition specificities) that are useful in the Prime editors of the disclosure are provided below in SEQ ID NOs 307-315, 322-324. In some embodiments, a prime editor comprises a Cas9 protein as according to any one of the sequences set forth in SEQ ID NOs: 307-315, 322-324 or a variant thereof. In some embodiments, the Cas9 protein is a Cas9 variant, for example, a SpCas9 variant (e.g., SpCas9-NG, SpCas9-NGA, SpRY, or SpG). In some embodiments, the Cas9 protein lacks the N-terminus methionine relative to a corresponding Cas9 protein (e.g., a Cas9 variant set forth in any one of SEQ ID NOs: 307, 310, 313, or 322). In some embodiments, a prime editor comprises a Cas9 protein (e.g., a Cas9 variant), having an amino acid sequence as according to any one of SEQ ID NOs: 307, 310, 313, or 322 not including the N-terminus methionine. In some embodiments, a prime editor comprises a Cas9 protein comprising one or more mutations (e.g., amino acid substitutions, insertions and/or deletions) relative to a corresponding Cas9 protein (e.g., a Cas9 protein set forth in any one of SEQ ID NOs: 307, 310, 313, or 322). In some embodiments, the Cas9 protein comprising one or mutations relative to a corresponding Cas9 protein comprises an amino acid sequence set forth in any one of SEQ ID NOs: 308, 309, 311, 312, 314, 315, 323, or 324.

In some embodiments, a Cas9 protein is a chimeric Cas9, e.g., modified Cas9, e.g., synthetic RNA-guided nucleases (sRGNs), e.g., modified by DNA family shuffling, e.g., sRGN3.1, sRGN3.3. In some embodiments, the DNA family shuffling comprises, fragmentation and reassembly of parental Cas9 genes, e.g., one or more of Cas9s from Staphylococcus hyicus (Shy), Staphylococcus lugdunensis (Slu), Staphylococcus microti (Smi), and Staphylococcus pasteuri (Spa). In some embodiments, a modified sluCas9 shows increased editing efficiency and/or specificity relative to a sluCas9 that is not modified. In some embodiments, a modified Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in editing efficiency compared to a Cas9 that is not modified. In some embodiments, a Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in specificity compared to a Cas9 that is not modified. In some embodiments, a Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in cleavage activity compared to a Cas9 that is not modified. In some embodiments, a Cas9, e.g., a sRGN shows ability to cleave a 5′-NNGG-3′ PAM-containing target. In some embodiments, a prime editor comprises a Cas9 protein (e.g., a chimeric Cas9), e.g., as according any one of the sequences set forth in SEQ ID NOs: 316-321, or a variant thereof. Exemplary amino acid sequences of Cas9 protein (e.g., sRGN) useful in the prime editors disclosed herein are provided below in SEQ ID NOs: 316-321. In some embodiments, a prime editor comprises a Cas9 protein, that lacks a N-terminus methionine relative to SEQ ID NO: 316 or SEQ ID NO: 319. In some embodiments, a prime editor comprises a Cas9 protein comprising one or more mutations (e.g., amino acid substitutions, insertions and/or deletions) relative to a corresponding Cas9 protein (e.g., a Cas9 protein set forth in any one of SEQ ID NOs: 316, or 319.). In some embodiments, the Cas9 protein comprising one or mutations relative to a corresponding Cas9 protein comprises an amino acid sequence set forth in any one of SEQ ID NOs: 317, 318, 320, or 321.

TABLE 9
Exemplary Cas protein sequences

| SEQ | | |
|---|---|---|
| ID | Sequence | |
| NO: | description | Amino acid sequence |
| 298 | wild type | MDKKYSIGLDIGTNSVGWAVITDEYKVPSKKFKVLGNTDRHSIKKNLIGALLFDSGE |
| | Streptococcus | TAEATRLKRTARRRYTRRKNRICYLQEIFSNEMAKVDDSFFHRLEESFLVEEDKKHE |
| | Pyogenes Cas9 | RHPIFGNIVDEVAYHEKYPTIYHLRKKLVDSTDKADLRLIYLALAHMIKFRGHFLIEG |
| | (SpCas9) | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 299 | SpCas9 H840A | MDKKYSIGLDIGTNSVGWAVITDEYKVPSKKFKVLGNTDRHSIKKNLIGALLFDSGE |
| | nickase | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 300 | Met (-) | DKKYSIGLDIGTNSVGWAVITDEYKVPSKKFKVLGNTDRHSIKKNLIGALLFDSGET |
| | SpCas9 | AEATRLKRTARRRYTRRKNRICYLQEIFSNEMAKVDDSFFHRLEESFLVEEDKKHER |
| | H840A nickase | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 301 | wild type | MNQKFILGLDIGITSVGYGLIDYETKNIIDAGVRLFPEANVENNEGRRSKRGSRRLKR |
| | Staphylococcus | RRIHRLERVKKLLEDYNLLDQSQIPQSTNPYAIRVKGLSEALSKDELVIALLHIAKRR |
| | lugdunensis | GIHKIDVIDSNDDVGNELSTKEQLNKNSKLLKDKFVCQIQLERMNEGQVRGEKNRF |
| | (Slu)Cas9 | KTADIIKEIIQLLNVQKNFHQLDENFINKYIELVEMRREYFEGPGKGSPYGWEGDPK |
| | | AWYETLMGHCTYFPDELRSVKYAYSADLFNALNDLNNLVIQRDGLSKLEYHEKYH |
| | | IIENVFKQKKKPTLKQIANEINVNPEDIKGYRITKSGKPQFTEFKLYHDLKSVLFDQSI |
| | | LENEDVLDQIAEILTIYQDKDSIKSKLTELDILLNEEDKENIAQLTGYTGTHRLSLKCI |
| | | RLVLEEQWYSSRNQMEIFTHLNIKPKKINLTAANKIPKAMIDEFILSPVVKRTFGQAI |
| | | NLINKIIEKYGVPEDIIIELARENNSKDKQKFINEMQKKNENTRKRINEIIGKYGNQNA |
| | | KRLVEKIRLHDEQEGKCLYSLESIPLEDLLNNPNHYEVDHIIPRSVSFDNSYHNKVLV |
| | | KQSENSKKSNLTPYQYFNSGKSKLSYNQFKQHILNLSKSQDRISKKKKEYLLEERDI |
| | | NKFEVQKEFINRNLVDTRYATRELTNYLKAYFSANNMNVKVKTINGSFTDYLRKV |
| | | WKFKKERNHGYKHHAEDALIIANADFLFKENKKLKAVNSVLEKPEIESKQLDIQVD |
| | | SEDNYSEMFIIPKQVQDIKDFRNFKYSHRVDKKPNRQLINDTLYSTRKKDNSTYIVQ |
| | | TIKDIYAKDNTTLKKQFDKSPEKFLMYQHDPRTFEKLEVIMKQYANEKNPLAKYHE |
| | | ETGEYLTKYSKKNNGPIVKSLKYIGNKLGSHLDVTHQFKSSTKKLVKLSIKPYRFDV |
| | | YLTDKGYKFITISYLDVLKKDNYYYIPEQKYDKLKLGKAIDKNAKFIASFYKNDLIK |
| | | LDGEIYKIIGVNSDTRNMIELDLPDIRYKEYCELNNIKGEPRIKKTIGKKVNSIEKLTT |
| | | DVLGNVFTNTQYTKPQLLFKRGN |
| 302 | SluCas9 | MNQKFILGLDIGITSVGYGLIDYETKNIIDAGVRLFPEANVENNEGRRSKRGSRRLKR |
| | N582A nickase | RRIHRLERVKKLLEDYNLLDQSQIPQSTNPYAIRVKGLSEALSKDELVIALLHIAKRR |
| | | GIHKIDVIDSNDDVGNELSTKEQLNKNSKLLKDKFVCQIQLERMNEGQVRGEKNRF |
| | | KTADIIKEIIQLLNVQKNFHQLDENFINKYIELVEMRREYFEGPGKGSPYGWEGDPK |
| | | AWYETLMGHCTYFPDELRSVKYAYSADLFNALNDLNNLVIQRDGLSKLEYHEKYH |
| | | IIENVFKQKKKPTLKQIANEINVNPEDIKGYRITKSGKPQFTEFKLYHDLKSVLFDQSI |
| | | LENEDVLDQIAEILTIYQDKDSIKSKLTELDILLNEEDKENIAQLTGYTGTHRLSLKCI |
| | | RLVLEEQWYSSRNQMEIFTHLNIKPKKINLTAANKIPKAMIDEFILSPVVKRTFGQAI |
| | | NLINKIIEKYGVPEDIIIELARENNSKDKQKFINEMQKKNENTRKRINEIIGKYGNQNA |
| | | KRLVEKIRLHDEQEGKCLYSLESIPLEDLLNNPNHYEVDHIIPRSVSFDNSYHNKVLV |
| | | KQSEASKKSNLTPYQYFNSGKSKLSYNQFKQHILNLSKSQDRISKKKKEYLLEERDI |
| | | NKFEVQKEFINRNLVDTRYATRELTNYLKAYFSANNMNVKVKTINGSFTDYLRKV |
| | | WKFKKERNHGYKHHAEDALIIANADFLFKENKKLKAVNSVLEKPEIESKQLDIQVD |
| | | SEDNYSEMFIIPKQVQDIKDFRNFKYSHRVDKKPNRQLINDTLYSTRKKDNSTYIVQ |
| | | TIKDIYAKDNTTLKKQFDKSPEKFLMYQHDPRTFEKLEVIMKQYANEKNPLAKYHE |
| | | ETGEYLTKYSKKNNGPIVKSLKYIGNKLGSHLDVTHQFKSSTKKLVKLSIKPYRFDV |
| | | YLTDKGYKFITISYLDVLKKDNYYYIPEQKYDKLKLGKAIDKNAKFIASFYKNDLIK |
| | | LDGEIYKIIGVNSDTRNMIELDLPDIRYKEYCELNNIKGEPRIKKTIGKKVNSIEKLTT |
| | | DVLGNVFTNTQYTKPQLLFKRGN |
| 303 | Met (-) | NQKFILGLDIGITSVGYGLIDYETKNIIDAGVRLFPEANVENNEGRRSKRGSRRLKRR |
| | SluCas9 | RIHRLERVKKLLEDYNLLDQSQIPQSTNPYAIRVKGLSEALSKDELVIALLHIAKRRGI |
| | nickase | HKIDVIDSNDDVGNELSTKEQLNKNSKLLKDKFVCQIQLERMNEGQVRGEKNRFKT |
| | | ADIIKEIIQLLNVQKNFHQLDENFINKYIELVEMRREYFEGPGKGSPYGWEGDPKAW |
| | | YETLMGHCTYFPDELRSVKYAYSADLFNALNDLNNLVIQRDGLSKLEYHEKYHIIE |
| | | NVFKQKKKPTLKQIANEINVNPEDIKGYRITKSGKPQFTEFKLYHDLKSVLFDQSILE |
| | | NEDVLDQIAEILTIYQDKDSIKSKLTELDILLNEEDKENIAQLTGYTGTHRLSLKCIRL |
| | | VLEEQWYSSRNQMEIFTHLNIKPKKINLTAANKIPKAMIDEFILSPVVKRTFGQAINLI |
| | | NKIIEKYGVPEDIIIELARENNSKDKQKFINEMQKKNENTRKRINEIIGKYGNQNAKR |
| | | LVEKIRLHDEQEGKCLYSLESIPLEDLLNNPNHYEVDHIIPRSVSFDNSYHNKVLVKQ |
| | | SEASKKSNLTPYQYFNSGKSKLSYNQFKQHILNLSKSQDRISKKKKEYLLEERDINKF |
| | | EVQKEFINRNLVDTRYATRELTNYLKAYFSANNMNVKVKTINGSFTDYLRKVWKF |
| | | KKERNHGYKHHAEDALIIANADFLFKENKKLKAVNSVLEKPEIESKQLDIQVDSEDN |
| | | YSEMFIIPKQVQDIKDFRNFKYSHRVDKKPNRQLINDTLYSTRKKDNSTYIVQTIKDI |
| | | YAKDNTTLKKQFDKSPEKFLMYQHDPRTFEKLEVIMKQYANEKNPLAKYHEETGE |
| | | YLTKYSKKNNGPIVKSLKYIGNKLGSHLDVTHQFKSSTKKLVKLSIKPYRFDVYLTD |
| | | KGYKFITISYLDVLKKDNYYYIPEQKYDKLKLGKAIDKNAKFIASFYKNDLIKLDGEI |
| | | YKIIGVNSDTRNMIELDLPDIRYKEYCELNNIKGEPRIKKTIGKKVNSIEKLTTDVLGN |
| | | VFTNTQYTKPQLLFKRGN |
| 304 | Staphylococcus | MKRNYILGLDIGITSVGYGIIDYETRDVIDAGVRLFKEANVENNEGRRSKRGARRLK |
| | aureus Cas9 | RRRRHRIQRVKKLLFDYNLLTDHSELSGINPYEARVKGLSQKLSEEEFSAALLHLAK |
| | (SaCas9) | RRGVHNVNEVEEDTGNELSTKEQISRNSKALEEKYVAELQLERLKKDGEVRGSINR |
| | | FKTSDYVKEAKQLLKVQKAYHQLDQSFIDTYIDLLETRRTYYEGPGEGSPFGWKDI |
| | | KEWYEMLMGHCTYFPEELRSVKYAYNADLYNALNDLNNLVITRDENEKLEYYEKF |
| | | QIIENVFKQKKKPTLKQIAKEILVNEEDIKGYRVTSTGKPEFTNLKVYHDIKDITARK |
| | | EIIENAELLDQIAKILTIYQSSEDIQEELTNLNSELTQEEIEQISNLKGYTGTHNLSLKAI |
| | | NLILDELWHTNDNQIAIFNRLKLVPKKVDLSQQKEIPTTLVDDFILSPVVKRSFIQSIK |
| | | VINAIIKKYGLPNDIIIELAREKNSKDAQKMINEMQKRNRQTNERIEEIIRTTGKENAK |
| | | YLIEKIKLHDMQEGKCLYSLEAIPLEDLLNNPFNYEVDHIIPRSVSFDNSFNNKVLVK |
| | | QEENSKKGNRTPFQYLSSSDSKISYETFKKHILNLAKGKGRISKTKKEYLLEERDINR |
| | | FSVQKDFINRNLVDTRYATRGLMNLLRSYFRVNNLDVKVKSINGGFTSFLRRKWKF |
| | | KKERNKGYKHHAEDALIIANADFIFKEWKKLDKAKKVMENQMFEEKQAESMPEIE |
| | | TEQEYKEIFITPHQIKHIKDFKDYKYSHRVDKKPNRELINDTLYSTRKDDKGNTLIVN |
| | | NLNGLYDKDNDKLKKLINKSPEKLLMYHHDPQTYQKLKLIMEQYGDEKNPLYKYY |
| | | EETGNYLTKYSKKDNGPVIKKIKYYGNKLNAHLDITDDYPNSRNKVVKLSLKPYRF |
| | | DVYLDNGVYKFVTVKNLDVIKKENYYEVNSKCYEEAKKLKKISNQAEFIASFYNND |
| | | LIKINGELYRVIGVNNDLLNRIEVNMIDITYREYLENMNDKRPPRIIKTIASKTQSIKK |
| | | YSTDILGNLYEVKSKKHPQIIKKG |
| 305 | SaCas9 N580A | MKRNYILGLDIGITSVGYGIIDYETRDVIDAGVRLFKEANVENNEGRRSKRGARRLK |
| | nickase | RRRRHRIQRVKKLLFDYNLLTDHSELSGINPYEARVKGLSQKLSEEEFSAALLHLAK |
| | | RRGVHNVNEVEEDTGNELSTKEQISRNSKALEEKYVAELQLERLKKDGEVRGSINR |
| | | FKTSDYVKEAKQLLKVQKAYHQLDQSFIDTYIDLLETRRTYYEGPGEGSPFGWKDI |
| | | KEWYEMLMGHCTYFPEELRSVKYAYNADLYNALNDLNNLVITRDENEKLEYYEKF |
| | | QIIENVFKQKKKPTLKQIAKEILVNEEDIKGYRVTSTGKPEFTNLKVYHDIKDITARK |
| | | EIIENAELLDQIAKILTIYQSSEDIQEELTNLNSELTQEEIEQISNLKGYTGTHNLSLKAI |
| | | NLILDELWHTNDNQIAIFNRLKLVPKKVDLSQQKEIPTTLVDDFILSPVVKRSFIQSIK |
| | | VINAIIKKYGLPNDIIIELAREKNSKDAQKMINEMQKRNRQTNERIEEIIRTTGKENAK |
| | | YLIEKIKLHDMQEGKCLYSLEAIPLEDLLNNPFNYEVDHIIPRSVSFDNSFNNKVLVK |
| | | QEEASKKGNRTPFQYLSSSDSKISYETFKKHILNLAKGKGRISKTKKEYLLEERDINR |
| | | FSVQKDFINRNLVDTRYATRGLMNLLRSYFRVNNLDVKVKSINGGFTSFLRRKWKF |
| | | KKERNKGYKHHAEDALIIANADFIFKEWKKLDKAKKVMENQMFEEKQAESMPEIE |
| | | TEQEYKEIFITPHQIKHIKDFKDYKYSHRVDKKPNRELINDTLYSTRKDDKGNTLIVN |
| | | NLNGLYDKDNDKLKKLINKSPEKLLMYHHDPQTYQKLKLIMEQYGDEKNPLYKYY |
| | | EETGNYLTKYSKKDNGPVIKKIKYYGNKLNAHLDITDDYPNSRNKVVKLSLKPYRF |
| | | DVYLDNGVYKFVTVKNLDVIKKENYYEVNSKCYEEAKKLKKISNQAEFIASFYNND |
| | | LIKINGELYRVIGVNNDLLNRIEVNMIDITYREYLENMNDKRPPRIIKTIASKTQSIKK |
| | | YSTDILGNLYEVKSKKHPQIIKKG |
| 306 | Met (-) | KRNYILGLDIGITSVGYGIIDYETRDVIDAGVRLFKEANVENNEGRRSKRGARRLKR |
| | SaCas9 | RRRHRIQRVKKLLFDYNLLTDHSELSGINPYEARVKGLSQKLSEEEFSAALLHLAKR |
| | nickase | RGVHNVNEVEEDTGNELSTKEQISRNSKALEEKYVAELQLERLKKDGEVRGSINRF |
| | | KTSDYVKEAKQLLKVQKAYHQLDQSFIDTYIDLLETRRTYYEGPGEGSPFGWKDIK |
| | | EWYEMLMGHCTYFPEELRSVKYAYNADLYNALNDLNNLVITRDENEKLEYYEKFQ |
| | | IIENVFKQKKKPTLKQIAKEILVNEEDIKGYRVTSTGKPEFTNLKVYHDIKDITARKEI |
| | | IENAELLDQIAKILTIYQSSEDIQEELTNLNSELTQEEIEQISNLKGYTGTHNLSLKAIN |
| | | LILDELWHTNDNQIAIFNRLKLVPKKVDLSQQKEIPTTLVDDFILSPVVKRSFIQSIKVI |
| | | NAIIKKYGLPNDIIIELAREKNSKDAQKMINEMQKRNRQTNERIEEIIRTTGKENAKY |
| | | LIEKIKLHDMQEGKCLYSLEAIPLEDLLNNPFNYEVDHIIPRSVSFDNSFNNKVLVKQ |
| | | EEASKKGNRTPFQYLSSSDSKISYETFKKHILNLAKGKGRISKTKKEYLLEERDINRFS |
| | | VQKDFINRNLVDTRYATRGLMNLLRSYFRVNNLDVKVKSINGGFTSFLRRKWKFK |
| | | KERNKGYKHHAEDALIIANADFIFKEWKKLDKAKKVMENQMFEEKQAESMPEIETE |
| | | QEYKEIFITPHQIKHIKDFKDYKYSHRVDKKPNRELINDTLYSTRKDDKGNTLIVNNL |
| | | NGLYDKDNDKLKKLINKSPEKLLMYHHDPQTYQKLKLIMEQYGDEKNPLYKYYEE |
| | | TGNYLTKYSKKDNGPVIKKIKYYGNKLNAHLDITDDYPNSRNKVVKLSLKPYRFDV |
| | | YLDNGVYKFVTVKNLDVIKKENYYEVNSKCYEEAKKLKKISNQAEFIASFYNNDLI |
| | | KINGELYRVIGVNNDLLNRIEVNMIDITYREYLENMNDKRPPRIIKTIASKTQSIKKYS |
| | | TDILGNLYEVKSKKHPQIIKKG |
| 307 | SpCas9-NG | MDKKYSIGLDIGTNSVGWAVITDEYKVPSKKFKVLGNTDRHSIKKNLIGALLFDSGE |
| | (VRVRFRR) | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| 308 | spCas9-NG | MDKKYSIGLDIGTNSVGWAVITDEYKVPSKKFKVLGNTDRHSIKKNLIGALLFDSGE |
| | (H840A_VRV | TAEATRLKRTARRRYTRRKNRICYLQEIFSNEMAKVDDSFFHRLEESFLVEEDKKHE |
| | RFRR) | RHPIFGNIVDEVAYHEKYPTIYHLRKKLVDSTDKADLRLIYLALAHMIKFRGHFLIEG |
| | Nickase | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| 309 | Met (-) | DKKYSIGLDIGTNSVGWAVITDEYKVPSKKFKVLGNTDRHSIKKNLIGALLFDSGET |
| | SpCas9-NG | AEATRLKRTARRRYTRRKNRICYLQEIFSNEMAKVDDSFFHRLEESFLVEEDKKHER |
| | Nickase | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| 310 | spCas9-NGA | MDKKYSIGLDIGTNSVGWAVITDEYKVPSKKFKVLGNTDRHSIKKNLIGALLFDSGE |
| | (VRQR) | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 311 | spCas9-NGA | MDKKYSIGLDIGTNSVGWAVITDEYKVPSKKFKVLGNTDRHSIKKNLIGALLFDSGE |
| | (H840A)_VRQ | TAEATRLKRTARRRYTRRKNRICYLQEIFSNEMAKVDDSFFHRLEESFLVEEDKKHE |
| | R) Nickase | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 312 | Met(-) | DKKYSIGLDIGTNSVGWAVITDEYKVPSKKFKVLGNTDRHSIKKNLIGALLFDSGET |
| | spCas9- | AEATRLKRTARRRYTRRKNRICYLQEIFSNEMAKVDDSFFHRLEESFLVEEDKKHER |
| | NGA VRQR | HPIFGNIVDEVAYHEKYPTIYHLRKKLVDSTDKADLRLIYLALAHMIKFRGHFLIEGD |
| | Nickase | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 313 | SpRY Cas9 | MDKKYSIGLDIGTNSVGWAVITDEYKVPSKKFKVLGNTDRHSIKKNLIGALLFDSGE |
| | | TAERTRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| 314 | SpRY Cas9 | MDKKYSIGLDIGTNSVGWAVITDEYKVPSKKFKVLGNTDRHSIKKNLIGALLFDSGE |
| | (H840A) | TAERTRLKRTARRRYTRRKNRICYLQEIFSNEMAKVDDSFFHRLEESFLVEEDKKHE |
| | Nickase | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| 315 | Met(-) SpRY | DKKYSIGLDIGTNSVGWAVITDEYKVPSKKFKVLGNTDRHSIKKNLIGALLFDSGET |
| | Cas9 Nickase | AERTRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| 316 | sRGN3.1 | MNQKFILGLDIGITSVGYGLIDYETKNIIDAGVRLFPEANVENNEGRRSKRGSRRLKR |
| | | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSENSKKSNLTPYQYFNSGKSKLSYNQFKQHILNLSKSQDRISKKKKEYLLEE |
| | | RDINKFEVQKEFINRNLVDTRYATRELTNYLKAYFSANNMNVKVKTINGSFTDYLR |
| | | KVWKFKKERNHGYKHHAEDALIIANADFLFKENKKLKAVNSVLEKPEIETKQLDIQ |
| | | VDSEDNYSEMFIIPKQVQDIKDFRNFKYSHRVDKKPNRQLINDTLYSTRKKDNSTYI |
| | | VQTIKDIYAKDNTTLKKQFDKSPEKFLMYQHDPRTFEKLEVIMKQYANEKNPLAKY |
| | | HEETGEYLTKYSKKNNGPIVKSLKYIGNKLGSHLDVTHQFKSSTKKLVKLSIKNYRF |
| | | DVYLTEKGYKFVTIAYLNVFKKDNYYYIPKDKYQELKEKKKIKDTDQFIASFYKND |
| | | LIKLNGDLYKIIGVNSDDRNIIELDYYDIKYKDYCEINNIKGEPRIKKTIGKKTESIEKF |
| | | TTDVLGNLYLHSTEKAPQLIFKRGL |
| 317 | sRGN3.1 | MNQKFILGLDIGITSVGYGLIDYETKNIIDAGVRLFPEANVENNEGRRSKRGSRRLKR |
| | (N585A) | RRIHRLERVKLLLTEYDLINKEQIPTSNNPYQIRVKGLSEILSKDELAIALLHLAKRRG |
| | Nickase | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSEASKKSNLTPYQYFNSGKSKLSYNQFKQHILNLSKSQDRISKKKKEYLLEE |
| | | RDINKFEVQKEFINRNLVDTRYATRELTNYLKAYFSANNMNVKVKTINGSFTDYLR |
| | | KVWKFKKERNHGYKHHAEDALIIANADFLFKENKKLKAVNSVLEKPEIETKQLDIQ |
| | | VDSEDNYSEMFIIPKQVQDIKDFRNFKYSHRVDKKPNRQLINDTLYSTRKKDNSTYI |
| | | VQTIKDIYAKDNTTLKKQFDKSPEKFLMYQHDPRTFEKLEVIMKQYANEKNPLAKY |
| | | HEETGEYLTKYSKKNNGPIVKSLKYIGNKLGSHLDVTHQFKSSTKKLVKLSIKNYRF |
| | | DVYLTEKGYKFVTIAYLNVFKKDNYYYIPKDKYQELKEKKKIKDTDQFIASFYKND |
| | | LIKLNGDLYKIIGVNSDDRNIIELDYYDIKYKDYCEINNIKGEPRIKKTIGKKTESIEKF |
| | | TTDVLGNLYLHSTEKAPQLIFKRGL |
| 318 | Met(-) | NQKFILGLDIGITSVGYGLIDYETKNIIDAGVRLFPEANVENNEGRRSKRGSRRLKRR |
| | sRGN3.1 | RIHRLERVKLLLTEYDLINKEQIPTSNNPYQIRVKGLSEILSKDELAIALLHLAKRRGI |
| | (N584A) | HNVDVAADKEETASDSLSTKDQINKNAKFLESRYVCELQKERLENEGHVRGVENRF |
| | Nickase | LTKDIVREAKKIIDTQMQYYPEIDETFKEKYISLVETRREYFEGPGQGSPFGWNGDLK |
| | | KWYEMLMGHCTYFPQELRSVKYAYSADLFNALNDLNNLIIQRDNSEKLEYHEKYHI |
| | | IENVFKQKKKPTLKQIAKEIGVNPEDIKGYRITKSGTPEFTSFKLFHDLKKVVKDHAI |
| | | LDDIDLLNQIAEILTIYQDKDSIVAELGQLEYLMSEADKQSISELTGYTGTHSLSLKC |
| | | MNMIIDELWHSSMNQMEVFTYLNMRPKKYELKGYQRIPTDMIDDAILSPVVKRTFI |
| | | QSINVINKVIEKYGIPEDIIIELARENNSDDRKKFINNLQKKNEATRKRINEIIGQTGNQ |
| | | NAKRIVEKIRLHDQQEGKCLYSLESIPLEDLLNNPNHYEVDHIIPRSVSFDNSYHNKV |
| | | LVKQSEASKKSNLTPYQYFNSGKSKLSYNQFKQHILNLSKSQDRISKKKKEYLLEER |
| | | DINKFEVQKEFINRNLVDTRYATRELTNYLKAYFSANNMNVKVKTINGSFTDYLRK |
| | | VWKFKKERNHGYKHHAEDALIIANADFLFKENKKLKAVNSVLEKPEIETKQLDIQV |
| | | DSEDNYSEMFIIPKQVQDIKDFRNFKYSHRVDKKPNRQLINDTLYSTRKKDNSTYIV |
| | | QTIKDIYAKDNTTLKKQFDKSPEKFLMYQHDPRTFEKLEVIMKQYANEKNPLAKYH |
| | | EETGEYLTKYSKKNNGPIVKSLKYIGNKLGSHLDVTHQFKSSTKKLVKLSIKNYRFD |
| | | VYLTEKGYKFVTIAYLNVFKKDNYYYIPKDKYQELKEKKKIKDTDQFIASFYKNDLI |
| | | KLNGDLYKIIGVNSDDRNIIELDYYDIKYKDYCEINNIKGEPRIKKTIGKKTESIEKFTT |
| | | DVLGNLYLHSTEKAPQLIFKRGL |
| 319 | sRGN3.3 | MNQKFILGLDIGITSVGYGLIDYETKNIIDAGVRLFPEANVENNEGRRSKRGSRRLKR |
| | | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSENSKKSNLTPYQYFNSGKSKLSYNQFKQHILNLSKSQDRISKKKKEYLLEE |
| | | RDINKFEVQKEFINRNLVDTRYATRELTSYLKAYFSANNMDVKVKTINGSFTNHLR |
| | | KVWRFDKYRNHGYKHHAEDALIIANADFLFKENKKLQNTNKILEKPTIENNTKKVT |
| | | VEKEEDYNNVFETPKLVEDIKQYRDYKFSHRVDKKPNRQLINDTLYSTRMKDEHDY |
| | | IVQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 320 | sRGN3.3 | MNQKFILGLDIGITSVGYGLIDYETKNIIDAGVRLFPEANVENNEGRRSKRGSRRLKR |
| | (N585A) | RRIHRLERVKLLLTEYDLINKEQIPTSNNPYQIRVKGLSEILSKDELAIALLHLAKRRG |
| | Nickase | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSEASKKSNLTPYQYFNSGKSKLSYNQFKQHILNLSKSQDRISKKKKEYLLEE |
| | | RDINKFEVQKEFINRNLVDTRYATRELTSYLKAYFSANNMDVKVKTINGSFTNHLR |
| | | KVWRFDKYRNHGYKHHAEDALIIANADFLFKENKKLQNTNKILEKPTIENNTKKVT |
| | | VEKEEDYNNVFETPKLVEDIKQYRDYKFSHRVDKKPNRQLINDTLYSTRMKDEHDY |
| | | IVQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 321 | Met(-) | NQKFILGLDIGITSVGYGLIDYETKNIIDAGVRLFPEANVENNEGRRSKRGSRRLKRR |
| | sRGN3.3 | RIHRLERVKLLLTEYDLINKEQIPTSNNPYQIRVKGLSEILSKDELAIALLHLAKRRGI |
| | (N584A) | HNVDVAADKEETASDSLSTKDQINKNAKFLESRYVCELQKERLENEGHVRGVENRF |
| | Nickase | LTKDIVREAKKIIDTQMQYYPEIDETFKEKYISLVETRREYFEGPGQGSPFGWNGDLK |
| | | KWYEMLMGHCTYFPQELRSVKYAYSADLFNALNDLNNLIIQRDNSEKLEYHEKYHI |
| | | IENVFKQKKKPTLKQIAKEIGVNPEDIKGYRITKSGTPEFTSFKLFHDLKKVVKDHAI |
| | | LDDIDLLNQIAEILTIYQDKDSIVAELGQLEYLMSEADKQSISELTGYTGTHSLSLKC |
| | | MNMIIDELWHSSMNQMEVFTYLNMRPKKYELKGYQRIPTDMIDDAILSPVVKRTFI |
| | | QSINVINKVIEKYGIPEDIIIELARENNSDDRKKFINNLQKKNEATRKRINEIIGQTGNQ |
| | | NAKRIVEKIRLHDQQEGKCLYSLESIPLEDLLNNPNHYEVDHIIPRSVSFDNSYHNKV |
| | | LVKQSEASKKSNLTPYQYFNSGKSKLSYNQFKQHILNLSKSQDRISKKKKEYLLEER |
| | | DINKFEVQKEFINRNLVDTRYATRELTSYLKAYFSANNMDVKVKTINGSFTNHLRK |
| | | VWRFDKYRNHGYKHHAEDALIIANADFLFKENKKLQNTNKILEKPTIENNTKKVTV |
| | | EKEEDYNNVFETPKLVEDIKQYRDYKFSHRVDKKPNRQLINDTLYSTRMKDEHDYI |
| | | VQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 322 | SpG | MDKKYSIGLDIGTNSVGWAVITDEYKVPSKKFKVLGNTDRHSIKKNLIGALLFDSGE |
| | | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 323 | SpG (H840A) | MDKKYSIGLDIGTNSVGWAVITDEYKVPSKKFKVLGNTDRHSIKKNLIGALLFDSGE |
| | Nickase | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 324 | Met(-) SpG | DKKYSIGLDIGTNSVGWAVITDEYKVPSKKFKVLGNTDRHSIKKNLIGALLFDSGET |
| | (H839A) | AEATRLKRTARRRYTRRKNRICYLQEIFSNEMAKVDDSFFHRLEESFLVEEDKKHER |
| | Nickase | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |

In some embodiments, a Cas9 protein comprises a variant Cas9 protein containing one or more amino acid substitutions. In some embodiments, a wildtype Cas9 protein comprises a RuvC domain and an HNH domain. In some embodiments, a prime editor comprises a nuclease active Cas9 protein that may cleave both strands of a double stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA, but can cleave only one strand of a double stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e., the PAM strand), but not the non-edit strand of a double stranded target DNA sequence. In some embodiments, a prime editor comprises a Cas9 having a non-functional RuvC domain that can cleave the target strand (i.e., the non-PAM strand), but not the edit strand of a double stranded target DNA sequence. In some embodiments, a prime editor comprises a Cas9 that has neither a functional RuvC domain nor a functional HNH domain, which may not cleave any strand of a double stranded target DNA sequence.

In some embodiments, a prime editor comprises a Cas9 having a mutation in the RuvC domain that reduces or abolishes the nuclease activity of the RuvC domain. In some embodiments, the Cas9 comprises a mutation at amino acid D10 as compared to a wild type SpCas9 as set forth in SEQ ID NO: 298, or a corresponding mutation thereof. In some embodiments, the Cas9 comprises a D10A mutation as compared to a wild type SpCas9 as set forth in SEQ ID NO: 298, or a corresponding mutation thereof. In some embodiments, the Cas9 polypeptide comprises a mutation at amino acid D10, G12, and/or G17 as compared to a wild type SpCas9 as set forth in SEQ ID NO: 298, or a corresponding mutation thereof. In some embodiments, the Cas9 polypeptide comprises a D10A mutation, a G12A mutation, and/or a G17A mutation as compared to a wild type SpCas9 as set forth in SEQ ID NO: 298, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprises a mutation at amino acid H840 as compared to a wild type SpCas9 as set forth in SEQ ID NO: 298, or a corresponding mutation thereof. In some embodiments, the Cas9 polypeptide comprises a H840A mutation as compared to a wild type SpCas9 as set forth in SEQ ID NO: 298, or a corresponding mutation thereof. In some embodiments, the Cas9 polypeptide comprises a mutation at amino acid E762, D839, H840, N854, N856, N863, H982, H983, A984, D986, and/or a A987 as compared to a wild type SpCas9 as set forth in SEQ ID NO: 298, or a corresponding mutation thereof. In some embodiments, the Cas9 polypeptide comprises a E762A, D839A, H840A, N854A, N856A, N863A, H982A, H983A, A984A, and/or a D986A mutation as compared to a wild type SpCas9 as set forth in SEQ ID NO: 298, or a corresponding mutation thereof. In some embodiments, the Cas9 polypeptide comprises a mutation at amino acid residue R221, N394, and/or H840 as compared to a wild type SpCas9 (e.g., SEQ ID NO: 298). In some embodiments, the Cas9 polypeptide comprises a R221K, N394L, and/or H840A mutation as compared to a wild type SpCas9 as set forth in SEQ ID NO: 298, or a corresponding mutation thereof. In some embodiments, the Cas9 polypeptide comprises a mutation at amino acid residue R220, N393, and/or H839 as compared to a wild type SpCas9 (e.g., SEQ ID NO: 298) lacking a N-terminal methionine, or a corresponding mutation thereof. In some embodiments, the Cas9 polypeptide comprises a R220K, N393K, and/or H839A mutation as compared to a wild type SpCas9 (as set forth in SEQ ID NO: 298) lacking a N-terminal methionine, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840X substitution and a DIOX mutation compared to a wild type SpCas9 as set forth in SEQ ID NO: 298 or corresponding mutations thereof, wherein X is any amino acid other than H for the H840X substitution and any amino acid other than D for the D10X substitution. In some embodiments, the dead Cas9 comprises a H840A and a D10A mutation as compared to a wild type SpCas9 as set forth in SEQ ID NO: 298, or corresponding mutations thereof.

In some embodiments, the N-terminal methionine is removed from the amino acid sequence of a Cas9 nickase, or from any Cas9 variant, ortholog, or equivalent disclosed or contemplated herein. For example, methionine-minus (Met (−)) Cas9 nickases include any one of the sequences set forth in SEQ ID NOs: 300, 303, 306, 309, 312, 315, 318, 321, 324, 325, or a variant thereof having an amino acid sequence that has at least 80%, at least 85%, at least 90%, at least 95%, or at least 99% sequence identity thereto.

Besides dead Cas9 and Cas9 nickase variants, the Cas9 proteins used herein may also include other Cas9 variants having at least about 70%, at least about 80%, at least about 90%, at least about 95%, at least about 96%, at least about 97%, at least about 98%, at least about 99%, at least about 99.5%, or at least about 99.9% sequence identity to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of a reference Cas9, e.g., a wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 99.5% of the amino acid length of a corresponding wild type Cas9.

In some embodiments, a Cas9 fragment is a functional fragment that retains one or more Cas9 activities. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length. In some embodiments, a prime editor comprises a Cas protein, e.g., Cas9, containing modifications that allow altered PAM recognition. In prime editing using a Cas-protein-based prime editor, a “protospacer adjacent motif (PAM)”, PAM sequence, or PAM-like motif, may be used to refer to a short DNA sequence immediately following the protospacer sequence on the PAM strand of the target gene. In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. Exemplary PAM sequences and corresponding Cas variants are described in Table 1 below. It should be appreciated that for each of the variants provided, the Cas protein comprises one or more of the amino acid substitutions as indicated compared to a wild type Cas protein sequence, for example, the Cas9 as set forth in SEQ ID NO: 298. The PAM motifs as shown in Table 1 below are in the order of 5′ to 3′.

In some embodiments, the Cas proteins of the disclosure can also be used to direct transcriptional control of target sequences, for example silencing transcription by sequence-specific binding to target sequences. In some embodiments, a Cas protein described herein may have one or mutations in a PAM recognition motif. In some embodiments, a Cas protein described herein may have altered PAM specificity.

As used in PAM sequences in Table 1, “N” refers to any one of nucleotides A, G, C, and T, “R” refers to nucleotide A or G, and “Y” refers to nucleotide C or T.

TABLE 1
Cas protein variants and corresponding PAM sequences

| Variant | PAM |
|---|---|
| spCas9 (wild type) | NGG, NGA, |
| | NAG, NGNGA |
| spCas9-VRVRFRR R1335V/L1111R/ | NG |
| D1135V/G1218R/E1219F/A1322R/T1337R | |
| spCas9-VQR (D1135V/R1335Q/T1337R ) | NGA |
| spCas9-EQR (D1135E/R1335Q/T1337R) | NGA |
| spCas9-VRER (D1135V/G1218R/R1335E/T1337R) | NGCG |
| spCas9-VRQR (D1135V, G1218R, R1335Q, T1337R) | NGA |
| Cas9-NG (L1111R, D1135V, G1218R, E1219F, | NGN |
| A1322R, T1337R, R1335V) | |
| SpG Cas9 (D1135L, S1136W, G1218K, E1219Q, | NGN |
| R1335Q, T1337R) | |
| SyRY Cas9 | NRN |
| (A61R, L1111R, N1317R, A1322R, and R1333P) | |
| xCas9 (E480K, E543D, E1219V, K294R, Q1256K, | NGN |
| A262T, S409I, M694I) | |
| SluCa9 | NNGG |
| sRGN1, sRGN2, sRGN4, sRGN3.1, sRGN3.3 | NNGG |
| saCas9 | NNGRRT, |
| | NNGRRN |
| saCas9-KKH (E782K, N968K, R1015H) | NNNRRT |
| spCas9-MQKSER (D1135M, S1136Q, G1218K, | NGCG/NGCN |
| E1219S, R1335E, T1337R) | |
| spCas9-LRKIQK (D1135L, S1136R, G1218K, | NGTN |
| E1219I, R1335Q, T1337K) | |
| spCas9-LRVSQK (D1135L, S1136R, G1218V, | NGTN |
| E1219S, R1335Q, T1337K) | |
| spCas9-LRVSQL(D1135L, S1136R, G1218V, | NGTN |
| E1219S, R1335Q, T1337L) | |
| Cpf1 | TTTV |
| Spy-Mac | NAA |
| NmCas9 | NNNNGATT |
| StCas9 | NNAGAAW |
| TdCas9 | NAAAAC |

In some embodiments, a prime editor comprises a Cas9 polypeptide comprising one or mutations selected from the group consisting of: A61R, L111R, D1135V, R221K, A262T, R324L, N394K, S4091, S4091, E427G, E480K, M495V, N497A, Y515N, K526E, F539S, E543D, R654L, R661A, R661L, R691A, N692A, M694A, M6941, Q695A, H698A, R753G, M7631, K848A, K890N, Q926A, K1003A, R1060A, L1111R, R1114G, D1135E, D1135L, D1135N, S1136W, V1139A, D1180G, G1218K, G1218R, G1218S, E1219Q, E1219V, E1219V, Q1221H, P1249S, E1253K, N1317R, A1320V, P1321S, A1322R, 11322V, D1332G, R1332N, A1332R, R1333K, R1333P, R1335L, R1335Q, R1335V, T1337N, T1337R, S1338T, H1349R, and any combinations thereof as compared to a wildtype SpCas9 polypeptide as set forth in SEQ ID NO: 298.

In some embodiments, a prime editor comprises a SaCas9 polypeptide. In some embodiments, the SaCas9 polypeptide comprises one or more of mutations E782K, N968K, and R1015H as compared to a wild type SaCas9. In some embodiments, a prime editor comprises a FnCas9 polypeptide, for example, a wildtype FnCas9 polypeptide or a FnCas9 polypeptide comprising one or more of mutations E1369R, E1449H, or R1556A as compared to the wild type FnCas9. In some embodiments, a prime editor comprises a Sc Cas9, for example, a wild type ScCas9 or a ScCas9 polypeptide comprises one or more of mutations I367K, G368D, I369K, H371L, T375S, T376G, and T1227K as compared to the wild type ScCas9. In some embodiments, a prime editor comprises a St1 Cas9 polypeptide, a St3 Cas9 polypeptide, or a SluCas9 polypeptide.

In some embodiments, a prime editor comprises a Cas polypeptide that comprises a circular permutant Cas variant. For example, a Cas9 polypeptide of a prime editor may be engineered such that the N-terminus and the C-terminus of a Cas9 protein (e.g., a wild type Cas9 protein, or a Cas9 nickase) are topically rearranged to retain the ability to bind DNA when complexed with a guide RNA (gRNA). An exemplary circular permutant configuration may be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

In some embodiments, prime editors described herein may also comprise Cas proteins other than Cas9. For example, in some embodiments, a prime editor as described herein may comprise a Cas12a (Cpf1) polypeptide or functional variants thereof. In some embodiments, the Cas12a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12a polypeptide.

In some embodiments, a prime editor comprises a Cas protein that is a Cas12b (C2c1) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c3) protein. In some embodiments, the Cas protein is a Cas12b nickase or a Cas12c nickase. In some embodiments, the Cas protein is a Cas12e, a Cas12d, a Cas13, Cas14a, Cas14b, Cas14c, Cas14d, Cas14e, Cas14f, Cas14g, Cas14h, Cas14u, or a Cas4 polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally-occurring Cas12e, Cas12d, Cas13, Cas14a, Cas14b, Cas14c, Cas14d, Cas14e, Cas14f, Cas14g, Cas14h, Cas14u, or Cas Φ protein. In some embodiments, the Cas protein is a Cas12e, Cas12d, Cas13, or Cas Φ nickase.

### Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase, that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing complex comprises at least one NLS. In some embodiments, a prime editor or prime editing complex comprises at least two NLSs. In embodiments with at least two NLSs, the NLSs can be the same NLS, or they can be different NLSs.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, the one or more NLSs of a prime editor are rich in lysine and arginine residues. In some embodiments, the one or more NLSs of a prime editor comprise proline residues. In some embodiments, a nuclear localization signal (NLS) comprises the sequence MDSLLMNRRKFLYQFKNVRWAKGRRETYLC (SEQ ID NO: 6).

In some embodiments, a NLS is a monopartite NLS. For example, in some embodiments, a NLS is a SV40 large T antigen NLS comprising the sequence PKKKRKV (SEQ ID NO: 4). In some embodiments, a NLS is a bipartite NLS. In some embodiments, a bipartite NLS comprises two basic domains separated by a spacer sequence comprising a variable number of amino acids. In some embodiments, a NLS is a bipartite NLS. In some embodiments, a bipartite NLS consists of two basic domains separated by a spacer sequence comprising a variable number of amino acids. In some embodiments, a NLS is a noncanonical sequences such as M9 of the hnRNP A1 protein, the influenza virus nucleoprotein NLS, and the yeast Gal4 protein NLS. In some embodiments, a NLS is a noncanonical sequences such as M9 of the hnRNP A1 protein, the influenza virus nucleoprotein NLS, and the yeast Gal4 protein NLS.

In some embodiments, a bipartite NLS consists of two basic domains separated by a spacer sequence comprising a variable number of amino acids. In some embodiments, a NLS comprises an amino acid sequence that is at least about 50%, 60%, 70%, 80%, 90%, 91%, 92%, 93%, 94%, 95%, 96%, 97%, 98%, 99%, or 100% identical to the amino acid sequence of any one of SEQ ID NOs: 4-14. In some embodiments, a NLS comprises an amino acid sequence selected from the group consisting of 4-14. In some embodiments, a prime editing composition comprises a polynucleotide that encodes a NLS that comprises an amino acid sequence that is at least about 50%, 60%, 70%, 80%, 90%, 91%, 92%, 93%, 94%, 95%, 96%, 97%, 98%, 99%, or 100% identical to the amino acid sequence of any one of SEQ ID NOs: 4-14. In some embodiments, a prime editing composition comprises a polynucleotide that encodes a NLS that comprises an amino acid sequence selected from the group consisting of 4-14.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, the one or more NLSs of a prime editor are rich in lysine and arginine residues. In some embodiments, the one or more NLSs of a prime editor comprise proline residues. Non-limiting examples of NLS sequences are provided in Table 2 below.

TABLE 2
Exemplary nuclear localization sequences

| | | SEQ |
|---|---|---|
| | | ID |
| Description | Sequence | NO: |
| NLS of SV40 | PKKKRKV | 4 |
| Large T-AG | | |
| NLS | MKRTADGSEFESPKKKRKV | 5 |
| NLS | MDSLLMNRRKFLYQFKNVRWAKGRRETYLC | 6 |
| NLS of | AVKRPAATKKAGQAKKKKLD | 7 |
| Nucleoplasmin | | |
| NLS of EGL-13 | MSRRRKANPTKLSENAKKLAKEVEN | 8 |
| NLS of C-Myc | PAAKRVKLD | 9 |
| NLS of Tus- | KLKIKRPVK | 10 |
| protein | | |
| NLS of polyoma | VSRKRPRP | 11 |
| large T-AG | | |
| NLS of | EGAPPAKRAR | 12 |
| Hepatitis D | | |
| virus antigen | | |
| NLS of murine | PPQPKKKPLDGE | 13 |
| p53 | | |
| C terminal | SGGSKRTADGSEFEPKKKRKV | 14 |
| linker and NLS | | |
| of an exemplary | | |
| prime editor | | |
| fusion protein | | |

In some embodiments, a prime editing complex comprises a fusion protein comprising a DNA binding domain (e.g., Cas9(H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 15. In some embodiments, the prime editing complex comprises a prime editor fusion protein that has the amino acid sequence of SEQ ID NO: 341. Sequence of an exemplary prime editor fusion protein comprising a DNA binding domain (e.g., Cas9(H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)] and its components are shown in Table 11.

In some embodiments, a prime editing complex comprises a fusion protein comprising a DNA binding domain (e.g., Cas9((R221K N394K H840A)) and a reverse transcriptase (e.g., a variant MMLV RT) having the following structure: [NLS]-[Cas9((R221K N394K H840A)]-[linker]-[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 342. In some embodiments, the prime editing complex comprises a prime editor fusion protein that has the amino acid sequence of SEQ ID NO: 343. Sequence of an exemplary prime editor fusion protein comprising a DNA binding domain (e.g., Cas9((R221K N394K H840A)) and a reverse transcriptase (e.g., a variant MMLV RT) having the following structure: [NLS]-[Cas9 (R221K N394K H840A)]-[linker]-[MMLV_RT(D200N)(T330P)(L603W)(T306K)(W313F)] and its components are shown in Table 12.

Polypeptides comprising components of a prime editor may be fused via peptide linkers or may be provided in trans relevant to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer fused or linked to a portion of the PEgRNA or ngRNA. For example, an MS2 coat protein fused or linked to the DNA polymerase and a MS2 hairpin installed on the PEgRNA for co-localization of the DNA polymerase and the RNA-guided DNA binding domain (e.g., a Cas9 nickase).

In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)n (SEQ ID NO: 326), (G)n (SEQ ID NO: 327), (EAAAK)n (SEQ ID NO: 328), (GGS)n (SEQ ID NO: 329), (SGGS)n (SEQ ID NO: 330), (XP)n (SEQ ID NO: 331), or any combination thereof, wherein n is independently an integer between 1 and 30, and wherein X is any amino acid. In some embodiments, the linker comprises the amino acid sequence (GGS)n (SEQ ID NO: 21632), wherein n is 1, 3, or 7. In some embodiments, the linker comprises the amino acid sequence SGSETPGTSESATPES (SEQ ID NO: 332). In some embodiments, the linker comprises the amino acid sequence SGGSSGGSSGSETPGTSESATPESSGGSSGGS (SEQ ID NO: 333). In some embodiments, the linker comprises the amino acid sequence SGGSGGSGGS (SEQ ID NO: 335). In some embodiments, the linker comprises the amino acid sequence SGGS (SEQ ID NO: 336). In other embodiments, the linker comprises the amino acid sequence

(SEQ ID NO: 337)
SGGSSGGSSGSETPGTSESATPESAGSYPYDVPDYAGSAAPAAKKKKLD
GSGSGGSSGGS.

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein, or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2-[DNA binding domain]-[polymerase]-COGH; or NH2-[polymerase]-[DNA binding domain]-COGH, wherein each instance of “]-[” indicates the presence of an optional linker sequence. In some embodiments, a prime editor comprises a fusion protein and a DNA polymerase domain provided in trans, wherein the fusion protein comprises the structure NH2-[DNA binding domain]-[RNA-protein recruitment polypeptide]-COGH. In some embodiments, a prime editor comprises a fusion protein and a DNA binding domain provided in trans, wherein the fusion protein comprises the structure NH2-[DNA polymerase domain]-[RNA-protein recruitment polypeptide]-COGH.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans splicing. In some embodiments, a prime editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete prime editor fusion protein in the target cell. In some embodiments, an exemplary protein described herein may lack a methionine residue at the N-terminus.

In some embodiments, a prime editor fusion protein comprises a Cas9(H840A) nickase and a wild type M-MLV RT. In some embodiments, a prime editor fusion protein comprises a Cas9(H840A) nickase and a M-MLV RT that comprises amino acid substitutions D200N, T330P, T306K, W313F, and L603W compared to a wild type M-MLV RT. In some embodiments, a prime editor fusion protein comprises a Cas9(H840A) nickase and a M-MLV RT that comprises amino acid substitutions D200N, T330P, T306K, W313F, and L603W compared to a wild type M-MLV RT. The amino acid sequence of an exemplary prime editor fusion protein and its individual components is shown in Table 11. In some embodiments, a prime editor fusion protein comprises a Cas9 (R221K N394K H840A) nickase and a M-MLV RT that comprises amino acid substitutions D200N, T330P, T306K, W313F, and L603W compared to a wild type M-MLV RT. The amino acid sequence of an exemplary Prime editor fusion protein and its individual components in shown in Table 12. In some embodiments an exemplary prime editor protein may comprise an amino acid sequence as set forth in any of the SEQ ID NO: 15, 341, 342, or 343.

In various embodiments, a prime editor fusion protein comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the prime editor fusion sequences described herein or known in the art.

Table 11 lists exemplary prime editor and its components

| SEQ | | |
|---|---|---|
| ID | | |
| NO. | DESCRIPTION | SEQUENCE |
| 15 | Exemplary Prime Editor | MKRTADGSEFESPKKKRKVDKKYSIGLDIGTNSVGWAVITDEYKV |
| | [NLS]-[Cas9(H840A)]- | PSKKFKVLGNTDRHSIKKNLIGALLFDSGETAEATRLKRTARRR |
| | [linker]- | YTRRKNRICYLQEIFSNEMAKVDDSFFHRLEESFLVEEDKKHER |
| | [MMLV_RT(D200N)(T330 | HPIFGNIVDEVAYHEKYPTIYHLRKKLVDSTDKADLRLIYLALAH |
| | P)(L603W)(T306K)(W313 | MIKFRGHFLIEGDLNPDNSDVDKLFIQLVQTYNQLFEENPINASG |
| | F)]-[NLS] | VDAKAILSARLSKSRRLENLIAQLPGEKKNGLFGNLIALSLGLTP |
| | | NFKSNFDLAEDAKLQLSKDTYDDDLDNLLAQIGDQYADLFLAAK |
| | | NLSDAILLSDILRVNTEITKAPLSASMIKRYDEHHQDLTLLKALV |
| | | RQQLPEKYKEIFFDQSKNGYAGYIDGGASQEEFYKFIKPILEKMD |
| | | GTEELLVKLNREDLLRKQRTFDNGSIPHQIHLGELHAILRRQEDF |
| | | YPFLKDNREKIEKILTFRIPYYVGPLARGNSRFAWMTRKSEETIT |
| | | PWNFEEVVDKGASAQSFIERMTNFDKNLPNEKVLPKHSLLYEYF |
| | | TVYNELTKVKYVTEGMRKPAFLSGEQKKAIVDLLFKTNRKVTV |
| | | KQLKEDYFKKIECFDSVEISGVEDRFNASLGTYHDLLKIIKDKDF |
| | | LDNEENEDILEDIVLTLTLFEDREMIEERLKTYAHLFDDKVMKQ |
| | | LKRRRYTGWGRLSRKLINGIRDKQSGKTILDFLKSDGFANRNFM |
| | | QLIHDDSLTFKEDIQKAQVSGQGDSLHEHIANLAGSPAIKKGILQ |
| | | TVKVVDELVKVMGRHKPENIVIEMARENQTTQKGQKNSRERM |
| | | KRIEEGIKELGSQILKEHPVENTQLQNEKLYLYYLQNGRDMYVD |
| | | QELDINRLSDYDVDAIVPQSFLKDDSIDNKVLTRSDKNRGKSDNV |
| | | PSEEVVKKMKNYWRQLLNAKLITQRKFDNLTKAERGGLSELDK |
| | | AGFIKRQLVETRQITKHVAQILDSRMNTKYDENDKLIREVKVITL |
| | | KSKLVSDFRKDFQFYKVREINNYHHAHDAYLNAVVGTALIKKYP |
| | | KLESEFVYGDYKVYDVRKMIAKSEQEIGKATAKYFFYSNIMNFF |
| | | KTEITLANGEIRKRPLIETNGETGEIVWDKGRDFATVRKVLSMP |
| | | QVNIVKKTEVQTGGFSKESILPKRNSDKLIARKKDWDPKKYGGF |
| | | DSPTVAYSVLVVAKVEKGKSKKLKSVKELLGITIMERSSFEKNPI |
| | | DFLEAKGYKEVKKDLIIKLPKYSLFELENGRKRMLASAGELQKG |
| | | NELALPSKYVNFLYLASHYEKLKGSPEDNEQKQLFVEQHKHYL |
| | | DEIIEQISEFSKRVILADANLDKVLSAYNKHRDKPIREQAENIIHLF |
| | | TLTNLGAPAAFKYFDTTIDRKRYTSTKEVLDATLIHQSITGLYET |
| | | RIDLSQLGGDSGGSSGGSSGSETPGTSESATPESSGGSSGGSSTLNI |
| | | EDEYRLHETSKEPDVSLGSTWLSDFPQAWAETGGMGLAVRQAPLIIPLK |
| | | ATSTPVSIKQYPMSQEARLGIKPHIQRLLDQGILVPCQSPWNTPLLPVKK |
| | | PGTNDYRPVQDLREVNKRVEDIHPTVPNPYNLLSGLPPSHQWYTVLDLK |
| | | DAFFCLRLHPTSQPLFAFEWRDPEMGISGQLTWTRLPQGFKNSPTLFNE |
| | | ALHRDLADFRIQHPDLILLQYVDDLLLAATSELDCQQGTRALLQTLGNL |
| | | GYRASAKKAQICQKQVKYLGYLLKEGQRWLTEARKETVMGQPTPKTPR |
| | | QLREFLGKAGFCRLFIPGFAEMAAPLYPLTKPGTLFNWGPDQQKAYQEI |
| | | KQALLTAPALGLPDLTKPFELFVDEKQGYAKGVLTQKLGPWRRPVAYLS |
| | | KKLDPVAAGWPPCLRMVAAIAVLTKDAGKLTMGQPLVILAPHAVEALVK |
| | | QPPDRWLSNARMTHYQALLLDTDRVQFGPVVALNPATLLPLPEEGLQH |
| | | NCLDILAEAHGTRPDLTDQPLPDADHTWYTDGSSLLQEGQRKAGAAVTT |
| | | ETEVIWAKALPAGTSAQRAELIALTQALKMAEGKKLNVYTDSRYAFATAH |
| | | IHGEIYRRRGWLTSEGKEIKNKDEILALLKALFLPKRLSIIHCPGHQKGHS |
| | | AEARGNRMADQAARKAAITETPDTSTLLIENSSPSGGSKRTADGSEFEPK |
| | | KKRKV |
| | | KEY: |
| | | NUCLEAR LOCALIZATION SEQUENCE (NLS) |
| | | CAS9(H840A) |
| | | 33-AMINO ACID LINKER |
| | | M-MLV REVERSE TRANSCRIPTASE |
| 341 | Met-Exemplary Prime | KRTADGSEFESPKKKRKVDKKYSIGLDIGTNSVGWAVITDEYKVP |
| | Editor [NLS]- | SKKFKVLGNTDRHSIKKNLIGALLFDSGETAEATRLKRTARRRY |
| | [Cas9(H840A)]-[linker]- | TRRKNRICYLQEIFSNEMAKVDDSFFHRLEESFLVEEDKKHERH |
| | [MMLV_RT(D200N)(T330 | PIFGNIVDEVAYHEKYPTIYHLRKKLVDSTDKADLRLIYLALAHM |
| | P)(L603W)(T306K)(W313 | IKFRGHFLIEGDLNPDNSDVDKLFIQLVQTYNQLFEENPINASGV |
| | F)]-[NLS] | DAKAILSARLSKSRRLENLIAQLPGEKKNGLFGNLIALSLGLTPN |
| | | FKSNFDLAEDAKLQLSKDTYDDDLDNLLAQIGDQYADLFLAAKN |
| | | LSDAILLSDILRVNTEITKAPLSASMIKRYDEHHQDLTLLKALVR |
| | | QQLPEKYKEIFFDQSKNGYAGYIDGGASQEEFYKFIKPILEKMD |
| | | GTEELLVKLNREDLLRKQRTFDNGSIPHQIHLGELHAILRRQEDF |
| | | YPFLKDNREKIEKILTFRIPYYVGPLARGNSRFAWMTRKSEETIT |
| | | PWNFEEVVDKGASAQSFIERMTNFDKNLPNEKVLPKHSLLYEYF |
| | | TVYNELTKVKYVTEGMRKPAFLSGEQKKAIVDLLFKTNRKVTV |
| | | KQLKEDYFKKIECFDSVEISGVEDRFNASLGTYHDLLKIIKDKDF |
| | | LDNEENEDILEDIVLTLTLFEDREMIEERLKTYAHLFDDKVMKQ |
| | | LKRRRYTGWGRLSRKLINGIRDKQSGKTILDFLKSDGFANRNFM |
| | | QLIHDDSLTFKEDIQKAQVSGQGDSLHEHIANLAGSPAIKKGILQ |
| | | TVKVVDELVKVMGRHKPENIVIEMARENQTTQKGQKNSRERM |
| | | KRIEEGIKELGSQILKEHPVENTQLQNEKLYLYYLQNGRDMYVD |
| | | QELDINRLSDYDVDAIVPQSFLKDDSIDNKVLTRSDKNRGKSDNV |
| | | PSEEVVKKMKNYWRQLLNAKLITQRKFDNLTKAERGGLSELDK |
| | | AGFIKRQLVETRQITKHVAQILDSRMNTKYDENDKLIREVKVITL |
| | | KSKLVSDFRKDFQFYKVREINNYHHAHDAYLNAVVGTALIKKYP |
| | | KLESEFVYGDYKVYDVRKMIAKSEQEIGKATAKYFFYSNIMNFF |
| | | KTEITLANGEIRKRPLIETNGETGEIVWDKGRDFATVRKVLSMP |
| | | QVNIVKKTEVQTGGFSKESILPKRNSDKLIARKKDWDPKKYGGF |
| | | DSPTVAYSVLVVAKVEKGKSKKLKSVKELLGITIMERSSFEKNPI |
| | | DFLEAKGYKEVKKDLIIKLPKYSLFELENGRKRMLASAGELQKG |
| | | NELALPSKYVNFLYLASHYEKLKGSPEDNEQKQLFVEQHKHYL |
| | | DEIIEQISEFSKRVILADANLDKVLSAYNKHRDKPIREQAENIIHLF |
| | | TLTNLGAPAAFKYFDTTIDRKRYTSTKEVLDATLIHQSITGLYET |
| | | RIDLSQLGGDSGGSSGGSSGSETPGTSESATPESSGGSSGGSSTLNI |
| | | EDEYRLHETSKEPDVSLGSTWLSDFPQAWAETGGMGLAVRQAPLIIPLK |
| | | ATSTPVSIKQYPMSQEARLGIKPHIQRLLDQGILVPCQSPWNTPLLPVKK |
| | | PGTNDYRPVQDLREVNKRVEDIHPTVPNPYNLLSGLPPSHQWYTVLDLK |
| | | DAFFCLRLHPTSQPLFAFEWRDPEMGISGQLTWTRLPQGFKNSPTLFNE |
| | | ALHRDLADFRIQHPDLILLQYVDDLLLAATSELDCQQGTRALLQTLGNL |
| | | GYRASAKKAQICQKQVKYLGYLLKEGQRWLTEARKETVMGQPTPKTPR |
| | | QLREFLGKAGFCRLFIPGFAEMAAPLYPLTKPGTLFNWGPDQQKAYQEI |
| | | KQALLTAPALGLPDLTKPFELFVDEKQGYAKGVLTQKLGPWRRPVAYLS |
| | | KKLDPVAAGWPPCLRMVAAIAVLTKDAGKLTMGQPLVILAPHAVEALVK |
| | | QPPDRWLSNARMTHYQALLLDTDRVQFGPVVALNPATLLPLPEEGLQH |
| | | NCLDILAEAHGTRPDLTDQPLPDADHTWYTDGSSLLQEGQRKAGAAVTT |
| | | ETEVIWAKALPAGTSAQRAELIALTQALKMAEGKKLNVYTDSRYAFATAH |
| | | IHGEIYRRRGWLTSEGKEIKNKDEILALLKALFLPKRLSIIHCPGHQKGHS |
| | | AEARGNRMADQAARKAAITETPDTSTLLIENSSPSGGSKRTADGSEFEPK |
| | | KKRKV |
| 5 | -N-terminal NLS | MKRTADGSEFESPKKKRKV |
| 300 | -CAS9 (H840A) | DKKYSIGLDIGTNSVGWAVITDEYKVPSKKFKVLGNTDRHSIKKNLI |
| | | GALLFDSGETAEATRLKRTARRRYTRRKNRICYLQEIFSNEMAKVD |
| | | DSFFHRLEESFLVEEDKKHERHPIFGNIVDEVAYHEKYPTIYHLRKKL |
| | | VDSTDKADLRLIYLALAHMIKFRGHFLIEGDLNPDNSDVDKLFIQLV |
| | | QTYNQLFEENPINASGVDAKAILSARLSKSRRLENLIAQLPGEKKNG |
| | | LFGNLIALSLGLTPNFKSNFDLAEDAKLQLSKDTYDDDLDNLLAQIG |
| | | DQYADLFLAAKNLSDAILLSDILRVNTEITKAPLSASMIKRYDEHHQ |
| | | DLTLLKALVRQQLPEKYKEIFFDQSKNGYAGYIDGGASQEEFYKFIK |
| | | PILEKMDGTEELLVKLNREDLLRKQRTFDNGSIPHQIHLGELHAILRR |
| | | QEDFYPFLKDNREKIEKILTFRIPYYVGPLARGNSRFAWMTRKSEETI |
| | | TPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQL |
| | | KEDYFKKIECFDSVEISGVEDRFNASLGTYHDLLKIIKDKDFLDNEEN |
| | | EDILEDIVLTLTLFEDREMIEERLKTYAHLFDDKVMKQLKRRRYTG |
| | | WGRLSRKLINGIRDKQSGKTILDFLKSDGFANRNFMQLIHDDSLTFK |
| | | EDIQKAQVSGQGDSLHEHIANLAGSPAIKKGILQTVKVVDELVKVM |
| | | GRHKPENIVIEMARENQTTQKGQKNSRERMKRIEEGIKELGSQILKE |
| | | HPVENTQLQNEKLYLYYLQNGRDMYVDQELDINRLSDYDVDAIVP |
| | | QSFLKDDSIDNKVLTRSDKNRGKSDNVPSEEVVKKMKNYWRQLLN |
| | | AKLITQRKFDNLTKAERGGLSELDKAGFIKRQLVETRQITKHVAQIL |
| | | DSRMNTKYDENDKLIREVKVITLKSKLVSDFRKDFQFYKVREINNY |
| | | HHAHDAYLNAVVGTALIKKYPKLESEFVYGDYKVYDVRKMIAKSE |
| | | QEIGKATAKYFFYSNIMNFFKTEITLANGEIRKRPLIETNGETGEIVW |
| | | DKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGI |
| | | TIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGD |
| 334 | -linker between CAS9 | SGGSSGGSSGSETPGTSESATPESSGGSSGGSS |
| | domain and RT domain | |
| | (33 amino acids) | |
| 3 | -MMLV_RT D200N | TLNIEDEYRLHETSKEPDVSLGSTWLSDFPQAWAETGGMGLAVRQA |
| | T330P L603W T306K | PLIIPLKATSTPVSIKQYPMSQEARLGIKPHIQRLLDQGILVPCQSPWN |
| | W313F | TPLLPVKKPGTNDYRPVQDLREVNKRVEDIHPTVPNPYNLLSGLPPS |
| | | HQWYTVLDLKDAFFCLRLHPTSQPLFAFEWRDPEMGISGQLTWTRL |
| | | PQGFKNSPTLFNEALHRDLADFRIQHPDLILLQYVDDLLLAATSELD |
| | | CQQGTRALLQTLGNLGYRASAKKAQICQKQVKYLGYLLKEGQRWL |
| | | TEARKETVMGQPTPKTPRQLREFLGKAGFCRLFIPGFAEMAAPLYPL |
| | | TKPGTLFNWGPDQQKAYQEIKQALLTAPALGLPDLTKPFELFVDEK |
| | | QGYAKGVLTQKLGPWRRPVAYLSKKLDPVAAGWPPCLRMVAAIA |
| | | VLTKDAGKLTMGQPLVILAPHAVEALVKQPPDRWLSNARMTHYQA |
| | | LLLDTDRVQFGPVVALNPATLLPLPEEGLQHNCLDILAEAHGTRPDL |
| | | TDQPLPDADHTWYTDGSSLLQEGQRKAGAAVTTETEVIWAKALPA |
| | | GTSAQRAELIALTQALKMAEGKKLNVYTDSRYAFATAHIHGEIYRR |
| | | RGWLTSEGKEIKNKDEILALLKALFLPKRLSIIHCPGHQKGHSAEAR |
| | | GNRMADQAARKAAITETPDTSTLLIENSSP |
| 14 | -C-terminal NLS | SGGSKRTADGSEFEPKKKRKV |

| SEQ | | |
|---|---|---|
| ID | | |
| NO. | DESCRIPTION | SEQUENCE |
| 342 | Exemplary prime editor | MKRTADGSEFESPKKKRKVDKKYSIGLDIGTNSVGWAVITDEYKV |
| | [NLS]-[Cas9((R220K) | PSKKFKVLGNTDRHSIKKNLIGALLFDSGETAEATRLKRTARRR |
| | (R393K)(H839A)]- | YTRRKNRICYLQEIFSNEMAKVDDSFFHRLEESFLVEEDKKHER |
| | [linker]- | HPIFGNIVDEVAYHEKYPTIYHLRKKLVDSTDKADLRLIYLALAH |
| | [MMLV_RT(D200N)(T33 | MIKFRGHFLIEGDLNPDNSDVDKLFIQLVQTYNQLFEENPINASG |
| | 0P)(L603W)(T306K)(W3 | VDAKAILSARLSKSRKLENLIAQLPGEKKNGLFGNLIALSLGLTP |
| | 13F)]-[NLS] | NFKSNFDLAEDAKLQLSKDTYDDDLDNLLAQIGDQYADLFLAAK |
| | | NLSDAILLSDILRVNTEITKAPLSASMIKRYDEHHQDLTLLKALV |
| | | RQQLPEKYKEIFFDQSKNGYAGYIDGGASQEEFYKFIKPILEKMD |
| | | GTEELLVKLKREDLLRKQRTFDNGSIPHQIHLGELHAILRRQED |
| | | FYPFLKDNREKIEKILTFRIPYYVGPLARGNSRFAWMTRKSEETI |
| | | TPWNFEEVVDKGASAQSFIERMTNFDKNLPNEKVLPKHSLLYEY |
| | | FTVYNELTKVKYVTEGMRKPAFLSGEQKKAIVDLLFKTNRKVT |
| | | VKQLKEDYFKKIECFDSVEISGVEDRFNASLGTYHDLLKIIKDKD |
| | | FLDNEENEDILEDIVLTLTLFEDREMIEERLKTYAHLFDDKVMK |
| | | QLKRRRYTGWGRLSRKLINGIRDKQSGKTILDFLKSDGFANRNF |
| | | MQLIHDDSLTFKEDIQKAQVSGQGDSLHEHIANLAGSPAIKKGIL |
| | | QTVKVVDELVKVMGRHKPENIVIEMARENQTTQKGQKNSRER |
| | | MKRIEEGIKELGSQILKEHPVENTQLQNEKLYLYYLQNGRDMY |
| | | VDQELDINRLSDYDVDAIVPQSFLKDDSIDNKVLTRSDKNRGKSD |
| | | NVPSEEVVKKMKNYWRQLLNAKLITQRKFDNLTKAERGGLSEL |
| | | DKAGFIKRQLVETRQITKHVAQILDSRMNTKYDENDKLIREVKV |
| | | ITLKSKLVSDFRKDFQFYKVREINNYHHAHDAYLNAVVGTALIK |
| | | KYPKLESEFVYGDYKVYDVRKMIAKSEQEIGKATAKYFFYSNIM |
| | | NFFKTEITLANGEIRKRPLIETNGETGEIVWDKGRDFATVRKVLS |
| | | MPQVNIVKKTEVQTGGFSKESILPKRNSDKLIARKKDWDPKKYG |
| | | GFDSPTVAYSVLVVAKVEKGKSKKLKSVKELLGITIMERSSFEKN |
| | | PIDFLEAKGYKEVKKDLIIKLPKYSLFELENGRKRMLASAGELQ |
| | | KGNELALPSKYVNFLYLASHYEKLKGSPEDNEQKQLFVEQHKH |
| | | YLDEIIEQISEFSKRVILADANLDKVLSAYNKHRDKPIREQAENII |
| | | HLFTLTNLGAPAAFKYFDTTIDRKRYTSTKEVLDATLIHQSITGL |
| | | YETRIDLSQLGGDSGGSSGGSKRTADGSEFESPKKKRKVSGGSSG |
| | | GSTLNIEDEYRLHETSKEPDVSLGSTWLSDFPQAWAETGGMGLAVRQAP |
| | | LIIPLKATSTPVSIKQYPMSQEARLGIKPHIQRLLDQGILVPCQSPWNTPLL |
| | | PVKKPGTNDYRPVQDLREVNKRVEDIHPTVPNPYNLLSGLPPSHQWYTV |
| | | LDLKDAFFCLRLHPTSQPLFAFEWRDPEMGISGQLTWTRLPQGFKNSPT |
| | | LFNEALHRDLADFRIQHPDLILLQYVDDLLLAATSELDCQQGTRALLQTL |
| | | GNLGYRASAKKAQICQKQVKYLGYLLKEGQRWLTEARKETVMGQPTPK |
| | | TPRQLREFLGKAGFCRLFIPGFAEMAAPLYPLTKPGTLFNWGPDQQKAY |
| | | QEIKQALLTAPALGLPDLTKPFELFVDEKQGYAKGVLTQKLGPWRRPVA |
| | | YLSKKLDPVAAGWPPCLRMVAAIAVLTKDAGKLTMGQPLVILAPHAVEA |
| | | LVKQPPDRWLSNARMTHYQALLLDTDRVQFGPVVALNPATLLPLPEEGL |
| | | QHNCLDILAEAHGTRPDLTDQPLPDADHTWYTDGSSLLQEGQRKAGAA |
| | | VTTETEVIWAKALPAGTSAQRAELIALTQALKMAEGKKLNVYTDSRYAFA |
| | | TAHIHGEIYRRRGWLTSEGKEIKNKDEILALLKALFLPKRLSIIHCPGHQK |
| | | GHSAEARGNRMADQAARKAAITETPDTSTLLIENSSPSGGSKRTADGSEF |
| | | ESPKKKRKVGSGPAAKRVKLD |
| | | KEY: |
| | | N-terminal bipartiteSV40NLS |
| | | CAS9(R221K N394K H840A) |
| | | SGGSx2-met-bpSV40NLS-SGGSx2 LINKER |
| | | M-MLV D200N T306K W313F T330P L603W REVERSE |
| | | TRANSCRIPTASE |
| | | C-terminal linker-NLS1 |
| | | C-terminal linker-NLS2 |
| 343 | Met-Exemplary prime | KRTADGSEFESPKKKRKVDKKYSIGLDIGTNSVGWAVITDEYKVPSK |
| | editor [NLS]- | KFKVLGNTDRHSIKKNLIGALLFDSGETAEATRLKRTARRRYTRRKN |
| | [Cas9((R220K)(R393K) | RICYLQEIFSNEMAKVDDSFFHRLEESFLVEEDKKHERHPIFGNIVDE |
| | (H839A)]-[linker]- | VAYHEKYPTIYHLRKKLVDSTDKADLRLIYLALAHMIKFRGHFLIEG |
| | [MMLV_RT(D200N)(T33 | DLNPDNSDVDKLFIQLVQTYNQLFEENPINASGVDAKAILSARLSKS |
| | 0P)(L603W)(T306K)(W3 | RKLENLIAQLPGEKKNGLFGNLIALSLGLTPNFKSNFDLAEDAKLQL |
| | 13F)]-[NLS] | SKDTYDDDLDNLLAQIGDQYADLFLAAKNLSDAILLSDILRVNTEIT |
| | | KAPLSASMIKRYDEHHQDLTLLKALVRQQLPEKYKEIFFDQSKNGY |
| | | AGYIDGGASQEEFYKFIKPILEKMDGTEELLVKLKREDLLRKQRTFD |
| | | NGSIPHQIHLGELHAILRRQEDFYPFLKDNREKIEKILTFRIPYYVGPL |
| | | ARGNSRFAWMTRKSEETITPWNFEEVVDKGASAQSFIERMTNFDKN |
| | | LPNEKVLPKHSLLYEYFTVYNELTKVKYVTEGMRKPAFLSGEQKKA |
| | | IVDLLFKTNRKVTVKQLKEDYFKKIECFDSVEISGVEDRFNASLGTY |
| | | HDLLKIIKDKDFLDNEENEDILEDIVLTLTLFEDREMIEERLKTYAHLF |
| | | DDKVMKQLKRRRYTGWGRLSRKLINGIRDKQSGKTILDFLKSDGFA |
| | | NRNFMQLIHDDSLTFKEDIQKAQVSGQGDSLHEHIANLAGSPAIKKG |
| | | ILQTVKVVDELVKVMGRHKPENIVIEMARENQTTQKGQKNSRERM |
| | | KRIEEGIKELGSQILKEHPVENTQLQNEKLYLYYLQNGRDMYVDQE |
| | | LDINRLSDYDVDAIVPQSFLKDDSIDNKVLTRSDKNRGKSDNVPSEE |
| | | VVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPK |
| | | YSLFELENGRKRMLASAGELQKGNELALPSKYVNFLYLASHYEKLK |
| | | GSPEDNEQKQLFVEQHKHYLDEIIEQISEFSKRVILADANLDKVLSAY |
| | | NKHRDKPIREQAENIIHLFTLTNLGAPAAFKYFDTTIDRKRYTSTKEV |
| | | LDATLIHQSITGLYETRIDLSQLGGDSGGSSGGSKRTADGSEFESPKK |
| | | KRKVSGGSSGGSTLNIEDEYRLHETSKEPDVSLGSTWLSDFPQAWAE |
| | | TGGMGLAVRQAPLIIPLKATSTPVSIKQYPMSQEARLGIKPHIQRLLD |
| | | QGILVPCQSPWNTPLLPVKKPGTNDYRPVQDLREVNKRVEDIHPTVP |
| | | NPYNLLSGLPPSHQWYTVLDLKDAFFCLRLHPTSQPLFAFEWRDPE |
| | | MGISGQLTWTRLPQGFKNSPTLFNEALHRDLADFRIQHPDLILLQYV |
| | | DDLLLAATSELDCQQGTRALLQTLGNLGYRASAKKAQICQKQVKY |
| | | LGYLLKEGQRWLTEARKETVMGQPTPKTPRQLREFLGKAGFCRLFIP |
| | | GFAEMAAPLYPLTKPGTLFNWGPDQQKAYQEIKQALLTAPALGLPD |
| | | LTKPFELFVDEKQGYAKGVLTQKLGPWRRPVAYLSKKLDPVAAGW |
| | | PPCLRMVAAIAVLTKDAGKLTMGQPLVILAPHAVEALVKQPPDRWL |
| | | SNARMTHYQALLLDTDRVQFGPVVALNPATLLPLPEEGLQHNCLDI |
| | | LAEAHGTRPDLTDQPLPDADHTWYTDGSSLLQEGQRKAGAAVTTE |
| | | TEVIWAKALPAGTSAQRAELIALTQALKMAEGKKLNVYTDSRYAFA |
| | | TAHIHGEIYRRRGWLTSEGKEIKNKDEILALLKALFLPKRLSIIHCPGH |
| | | QKGHSAEARGNRMADQAARKAAITETPDTSTLLIENSSPSGGSKRTA |
| | | DGSEFESPKKKRKVGSGPAAKRVKLD |
| 5 | -N-terminal bpSV40NLS | MKRTADGSEFESPKKKRKV |
| 325 | -CAS9 (R221K N394K | DKKYSIGLDIGTNSVGWAVITDEYKVPSKKFKVLGNTDRHSIKKNLI |
| | H840A) | GALLFDSGETAEATRLKRTARRRYTRRKNRICYLQEIFSNEMAKVD |
| | | DSFFHRLEESFLVEEDKKHERHPIFGNIVDEVAYHEKYPTIYHLRKKL |
| | | VDSTDKADLRLIYLALAHMIKFRGHFLIEGDLNPDNSDVDKLFIQLV |
| | | QTYNQLFEENPINASGVDAKAILSARLSKSRKLENLIAQLPGEKKNG |
| | | LFGNLIALSLGLTPNFKSNFDLAEDAKLQLSKDTYDDDLDNLLAQIG |
| | | DQYADLFLAAKNLSDAILLSDILRVNTEITKAPLSASMIKRYDEHHQ |
| | | DLTLLKALVRQQLPEKYKEIFFDQSKNGYAGYIDGGASQEEFYKFIK |
| | | PILEKMDGTEELLVKLKREDLLRKQRTFDNGSIPHQIHLGELHAILRR |
| | | QEDFYPFLKDNREKIEKILTFRIPYYVGPLARGNSRFAWMTRKSEETI |
| | | TPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQL |
| | | KEDYFKKIECFDSVEISGVEDRFNASLGTYHDLLKIIKDKDFLDNEEN |
| | | EDILEDIVLTLTLFEDREMIEERLKTYAHLFDDKVMKQLKRRRYTG |
| | | WGRLSRKLINGIRDKQSGKTILDFLKSDGFANRNFMQLIHDDSLTFK |
| | | EDIQKAQVSGQGDSLHEHIANLAGSPAIKKGILQTVKVVDELVKVM |
| | | GRHKPENIVIEMARENQTTQKGQKNSRERMKRIEEGIKELGSQILKE |
| | | HPVENTQLQNEKLYLYYLQNGRDMYVDQELDINRLSDYDVDAIVP |
| | | QSFLKDDSIDNKVLTRSDKNRGKSDNVPSEEVVKKMKNYWRQLLN |
| | | AKLITQRKFDNLTKAERGGLSELDKAGFIKRQLVETRQITKHVAQIL |
| | | DSRMNTKYDENDKLIREVKVITLKSKLVSDFRKDFQFYKVREINNY |
| | | HHAHDAYLNAVVGTALIKKYPKLESEFVYGDYKVYDVRKMIAKSE |
| | | QEIGKATAKYFFYSNIMNFFKTEITLANGEIRKRPLIETNGETGEIVW |
| | | DKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGI |
| | | TIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGD |
| 338 | -SGGSx2-bpSV40NLS- | SGGSSGGSKRTADGSEFESPKKKRKVSGGSSGGS |
| | SGGSx2 linker | |
| 3 | -MMLV_RT D200N | TLNIEDEYRLHETSKEPDVSLGSTWLSDFPQAWAETGGMGLAVRQA |
| | T330P L603W T306K | PLIIPLKATSTPVSIKQYPMSQEARLGIKPHIQRLLDQGILVPCQSPWN |
| | W313F | TPLLPVKKPGTNDYRPVQDLREVNKRVEDIHPTVPNPYNLLSGLPPS |
| | | HQWYTVLDLKDAFFCLRLHPTSQPLFAFEWRDPEMGISGQLTWTRL |
| | | PQGFKNSPTLFNEALHRDLADFRIQHPDLILLQYVDDLLLAATSELD |
| | | CQQGTRALLQTLGNLGYRASAKKAQICQKQVKYLGYLLKEGQRWL |
| | | TEARKETVMGQPTPKTPRQLREFLGKAGFCRLFIPGFAEMAAPLYPL |
| | | TKPGTLFNWGPDQQKAYQEIKQALLTAPALGLPDLTKPFELFVDEK |
| | | QGYAKGVLTQKLGPWRRPVAYLSKKLDPVAAGWPPCLRMVAAIA |
| | | VLTKDAGKLTMGQPLVILAPHAVEALVKQPPDRWLSNARMTHYQA |
| | | LLLDTDRVQFGPVVALNPATLLPLPEEGLQHNCLDILAEAHGTRPDL |
| | | TDQPLPDADHTWYTDGSSLLQEGQRKAGAAVTTETEVIWAKALPA |
| | | GTSAQRAELIALTQALKMAEGKKLNVYTDSRYAFATAHIHGEIYRR |
| | | RGWLTSEGKEIKNKDEILALLKALFLPKRLSIIHCPGHQKGHSAEAR |
| | | GNRMADQAARKAAITETPDTSTLLIENSSP |
| 340 | C-terminal linker-NLS | SGGSKRTADGSEFESPKKKRKV |
| 345 | C-terminal linker-NLS2 | GSGPAAKRVKLD |

### PEgRNA for Editing of CFTR Gene

The term “prime editing guide RNA”, or “PEgRNA”, refers to a guide polynucleotide that comprises one or more intended nucleotide edits for incorporation into the target DNA. In some embodiments, the PEgRNA associates with and directs a prime editor to incorporate the one or more intended nucleotide edits into the target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene, or may refer to the edit encoded by the editing template on the newly synthesized single stranded DNA that replaces the editing target sequence, as compared to the editing target sequence. In some embodiments, a PEgRNA comprises a spacer sequence that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA further comprises an extended nucleotide sequence comprising one or more intended nucleotide edits compared to the endogenous sequence of the target gene, wherein the extended nucleotide sequence may be referred to as an extension arm.

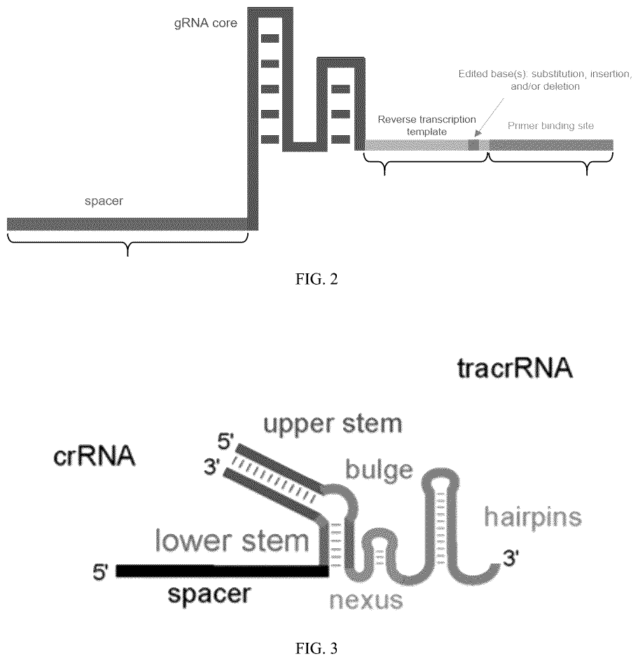

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the extension arm further comprises an editing template that comprises one or more intended nucleotide edits to be incorporated in the target gene by prime editing. In some embodiments, the editing template is a template for an RNA-dependent DNA polymerase domain or polypeptide of the prime editor, for example, a reverse transcriptase domain. The reverse transcriptase editing template may also be referred to herein as an RT template, or RTT. In some embodiments, the editing template comprises partial complementarity to an editing target sequence in the target gene, e.g., an CFTR gene. In some embodiments, the editing template comprises substantial or partial complementarity to the editing target sequence except at the position of the intended nucleotide edits to be incorporated into the target gene. An exemplary architecture of a PEgRNA including its components is as demonstrated in FIG. 2.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer sequence, the gRNA core, or the extension arm. In some embodiments, a PEgRNA comprises DNA in the spacer sequence. In some embodiments, the entire spacer sequence of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PEgRNA, the 3′ portion of the PEgRNA, or in the middle of the gRNA core. In some embodiments, a PEgRNA comprises a PBS and an editing template sequence in 5′ to 3′ order. In some embodiments, the gRNA core of a PEgRNA of this disclosure may be located in between a spacer and an extension arm of the PEgRNA. In some embodiments, the gRNA core of a PEgRNA may be located at the 3′ end of a spacer. In some embodiments, the gRNA core of a PEgRNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, the PEgRNA comprises, from 5′ to 3′: a spacer, a gRNA core, and an extension arm. In some embodiments, the PEgRNA comprises, from 5′ to 3′: a spacer, a gRNA core, an editing template, and a PBS. In some embodiments, the PEgRNA comprises, from 5′ to 3′: an extension arm, a spacer, and a gRNA core. In some embodiments, the PEgRNA comprises, from 5′ to 3′: an editing template, a PBS, a spacer, and a gRNA core.

In some embodiments, a PEgRNA comprises a single polynucleotide molecule that comprises the spacer sequence, the gRNA core, and the extension arm. In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA may comprise a first polynucleotide comprising the spacer and a first portion of a gRNA core comprising, which may also be referred to as a crRNA. In some embodiments, the PEgRNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem, as exemplified in FIG. 3.

In some embodiments, a spacer sequence comprises a region that has substantial complementarity to a search target sequence on the target strand of a double stranded target DNA, e.g., an CFTR gene. In some embodiments, the spacer sequence of a PEgRNA is identical or substantially identical to a protospacer sequence on the edit strand of the target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the target gene. In some embodiments, the spacer comprises is substantially complementary to the search target sequence.

In some embodiments, the length of the spacer varies from about 10 nucleotides to about 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, or 20 to 30 nucleotides in length. In some embodiments, the spacer is 20 nucleotides in length. In some embodiments, the spacer is 17 to 18 nucleotides in length. In some embodiments, the spacer is 20 nucleotides in length.

As used herein in a PEgRNA or a nick guide RNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PEgRNA or guide RNA or any chemically modified uracil nucleobase known in the art, such as 5-methoxyuracil.

Exemplary sequences for PEgRNA spacers are provided in Table 3A.

The extension arm of a PEgRNA may comprise a primer binding site (PBS) and an editing template (e.g., an RTT). The extension arm may be partially complementary to the spacer. In some embodiments, the editing template (e.g., RTT) is partially complementary to the spacer. In some embodiments, the editing template (e.g., RTT) and the primer binding site (PBS) are each partially complementary to the spacer.

An extension arm of a PEgRNA may comprise a primer binding site sequence (PBS, or PBS sequence) that comprises complementarity to and can hybridize with a free 3′ end of a single stranded DNA in the target gene (e.g., the CFTR gene) generated by nicking with a prime editor at the nick site on the PAM strand. The length of the PBS sequence may vary depending on, e.g., the prime editor components, the search target sequence and other components of the PEgRNA. In some embodiments, the length of the primer binding site (PBS) varies from at least 2 nucleotides to 50 nucleotides. For examples, a primer binding site (PBS) may be at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, or at least 50 nucleotides in length. In some embodiments, the PBS is at least 6 nucleotides in length. In some embodiments, the PBS is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is about 7 to 15 nucleotides in length. In some embodiments, the PBS is 6, 7, 8, 9, 10, 11, 12, 13, 14, 15, 16, 17, 18, 19, or 20 nucleotides in length. In some embodiments, the PBS is 8, 9, 10, 11, 12, 13, or 14 nucleotides in length

The PBS may be complementary or substantially complementary to a DNA sequence in the edit strand of the target gene. By annealing with the edit strand at a free hydroxy group, e.g., a free 3′ end generated by prime editor nicking, the PBS may initiate synthesis of a new single stranded DNA encoded by the editing template at the nick site. In some embodiments, the PBS is at least about 70%, 75%, 80%, 85%, 90%, 95%, or 100% complementary to a region of the edit strand of the target gene (e.g., the CFTR gene). In some embodiments, the PBS is perfectly complementary, or 100% complementary, to a region of the edit strand of the target gene (e.g., the CFTR gene).

Exemplary sequences for PBS are provided in Table 3B.

An extension arm of a PEgRNA may comprise an editing template that serves as a DNA synthesis template for the DNA polymerase in a prime editor during prime editing.

The length of an editing template may vary depending on, e.g., the prime editor components, the search target sequence and other components of the PEgRNA. In some embodiments, the editing template serves as a DNA synthesis template for a reverse transcriptase, and the editing template is referred to as a reverse transcription editing template (RTT).

The editing template (e.g., RTT), in some embodiments, is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the RTT is 12, 13, 14, 15, 16, 17, 18, 19, 20, 21, 22, 23, 24, or 25 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, or 40 nucleotides in length.

Exemplary sequences for RTT are provided in Table 3C.

In some embodiments, the editing template (e.g., RTT) sequence is about 70%, 75%, 80%, 85%, 90%, 95%, or 99% complementary to the editing target sequence on the edit strand of the target gene. In some embodiments, the editing template sequence (e.g., RTT) is substantially complementary to the editing target sequence. In some embodiments, the editing template sequence (e.g., RTT) is complementary to the editing target sequence except at positions of the intended nucleotide edits to be incorporated int the target gene. In some embodiments, the editing template comprises a nucleotide sequence comprising about 85% to about 95% complementarity to an editing target sequence in the edit strand in the target gene (e.g., the CFTR gene). In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the target gene (e.g., the CFTR gene).

An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 1, at least 2, at least 3, at least 4, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA may comprise one or more intended nucleotide edits, compared to the CFTR gene to be edited. Position of the intended nucleotide edit(s) relevant to other components of the PEgRNA, or to particular nucleotides (e.g., mutations) in the CFTR target gene may vary. In some embodiments, the nucleotide edit is in a region of the PEgRNA corresponding to or homologous to the protospacer sequence. In some embodiments, the nucleotide edit is in a region of the PEgRNA corresponding to a region of the CFTR gene outside of the protospacer sequence.

In some embodiments, the position of a nucleotide edit incorporation in the target gene may be determined based on position of the protospacer adjacent motif (PAM). For instance, the intended nucleotide edit may be installed in a sequence corresponding to the protospacer adjacent motif (PAM) sequence. In some embodiments, a nucleotide edit in the editing template is at a position corresponding to the 5′ most nucleotide of the PAM sequence. In some embodiments, a nucleotide edit in the editing template is at a position corresponding to the 3′ most nucleotide of the PAM sequence. In some embodiments, position of an intended nucleotide edit in the editing template may be referred to by aligning the editing template with the partially complementary edit strand of the target gene and referring to nucleotide positions on the editing strand where the intended nucleotide edit is incorporated. In some embodiments, a nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream of the 5′ most nucleotide of the PAM sequence in the edit strand of the target gene. By 0 nucleotide upstream or downstream of a reference position, it is meant that the intended nucleotide is immediately upstream or downstream of the reference position.

In some embodiments, a nucleotide edit is incorporated at a position corresponding to about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream of the 5′ most nucleotide of the PAM sequence. In some embodiments, the nucleotide edit is incorporated at a position corresponding to 3 nucleotides upstream of the 5′ most nucleotide of the PAM sequence. In some embodiments, the nucleotide edit in is incorporated at a position corresponding to 4 nucleotides upstream of the 5′ most nucleotide of the PAM sequence. In some embodiments, the nucleotide edit is incorporated at a position corresponding to 5 nucleotides upstream of the 5′ most nucleotide of the PAM sequence. In some embodiments, the nucleotide edit in the editing template is at a position corresponding to 6 nucleotides upstream of the 5′ most nucleotide of the PAM sequence.

In some embodiments, an intended nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides downstream of the 5′ most nucleotide of the PAM sequence in the edit strand of the target gene. In some embodiments, a nucleotide edit is incorporated at a position corresponding to about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides downstream of the 5′ most nucleotide of the PAM sequence. In some embodiments, a nucleotide edit is incorporated at a position corresponding to 3 nucleotides downstream of the 5′ most nucleotide of the PAM sequence. In some embodiments, a nucleotide edit is incorporated at a position corresponding to 4 nucleotides downstream of the 5′ most nucleotide of the PAM sequence. In some embodiments, a nucleotide edit is incorporated at a position corresponding to 5 nucleotides downstream of the 5′ most nucleotide of the PAM sequence. In some embodiments, a nucleotide edit is incorporated at a position corresponding to 6 nucleotides downstream of the 5′ most nucleotide of the PAM sequence. By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

When referred to within the PEgRNA, positions of the one or more intended nucleotide edits may be referred to relevant to components of the PEgRNA. For example, an intended nucleotide edit may be 5′ or 3′ to the PBS. In some embodiments, a PEgRNA comprises the structure, from 5′ to 3′: a spacer, a gRNA core, an editing template, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream to the 5′ most nucleotide of the PBS. In some embodiments, the intended nucleotide edit is 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream to the 5′ most nucleotide of the PBS.

The corresponding positions of the intended nucleotide edit incorporated in the target gene may also be referred to based on the nicking position generated by a prime editor based on sequence homology and complementarity. For example, in embodiments, the distance between the nucleotide edit to be incorporated into the target CFTR gene and the nick site (also referred to as the “nick to edit distance”) may be determined by the position of the nick site and the position of the nucleotide(s) corresponding to the intended nucleotide edit(s), for example, by identifying sequence complementarity between the spacer and the search target sequence and sequence complementarity between the editing template and the editing target sequence. In certain embodiments, the position of the nucleotide edit can be in any position downstream of the nick site on the edit strand (or the PAM strand) generated by the prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 base pair from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence. In some embodiments, the editing template extends beyond a nucleotide edit to be incorporated to the target CFTR gene sequence. For example, in some embodiments, the editing template comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs 3′ to the nucleotide edit to be incorporated to the target CFTR gene sequence. In some embodiments, the editing template comprises 1 to 80 nucleotides 3′ to the nucleotide edit to be incorporated to the target CFTR gene sequence.

In some embodiments, the editing template comprises at least 4 to 30 base pairs 3′ to the nucleotide edit to be incorporated to the target CFTR gene sequence. In some embodiments, the editing template comprises at least 4 to 25 base pairs 3′ to the nucleotide edit to be incorporated to the target CFTR gene sequence. In some embodiments, the editing template comprises at least 4 to 20 base pairs 3′ to the nucleotide edit to be incorporated to the target CFTR gene sequence. In some embodiments, the editing template comprises at least 4 to 30 base pairs 5′ to the nucleotide edit to be incorporated to the target CFTR gene sequence. In some embodiments, the editing template comprises at least 4 to 25 base pairs 5′ to the nucleotide edit to be incorporated to the target CFTR gene sequence. In some embodiments, the editing template comprises at least 4 to 20 base pairs 5′ to the nucleotide edit to be incorporated to the target CFTR gene sequence.

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nucleobase position (e.g., for a PEgRNA following 5′-spacer-gRNA core-RTT-PBS-3′ orientation, the 5′ most nucleobase is the “first base”). In some embodiments, the editing template comprises an uracil at the first nucleobase position (e.g., for a PEgRNA following 5′-spacer-gRNA core-RTT-PBS-3′ orientation, the 5′ most nucleobase is the “first base”). In some embodiments, the editing template comprises a cytosine at the first nucleobase position (e.g., for a PEgRNA following 5′-spacer-gRNA core-RTT-PBS-3′ orientation, the 5′ most nucleobase is the “first base”). In some embodiments, the editing template does not comprise a cytosine at the first nucleobase position (e.g., for a PEgRNA following 5′-spacer-gRNA core-RTT-PBS-3′ orientation, the 5′ most nucleobase is the “first base”).

The editing template of a PEgRNA may encode a new single stranded DNA (e.g., by reverse transcription) to replace an editing target sequence in the target gene. In some embodiments, the editing target sequence in the edit strand of the target gene is replaced by the newly synthesized strand, and the nucleotide edit(s) are incorporated in the region of the target gene. In some embodiments, the target gene is an CFTR gene. In some embodiments, the editing template of the PEgRNA encodes a newly synthesized single stranded DNA that comprises a wild type CFTR gene sequence. In some embodiments, the newly synthesized DNA strand replaces the editing target sequence in the target CFTR gene, wherein the editing target sequence (or the endogenous sequence complementary to the editing target sequence on the target strand of the CFTR gene) comprises a mutation or a nucleotide alteration compared to a wild type CFTR gene. In some embodiments, the mutation is associated with cystic fibrosis.

In some embodiments, the newly synthesized single stranded DNA encoded by the editing template replaces the editing target sequence and corrects the mutation in the editing target sequence of the target CFTR gene.

In some embodiments, the editing target sequence comprises a mutation in a sequence of the CFTR gene that encodes a MSD1 domain of the CFTR protein. In some embodiments, the editing target sequence comprises a mutation in a sequence of the CFTR gene that encodes a NBD1 domain of the CFTR protein. In some embodiments, the editing target sequence comprises a mutation in a sequence of the CFTR gene that encodes a R domain of the CFTR protein. In some embodiments, the editing target sequence comprises a mutation in a sequence of the CFTR gene that encodes a NBD2 domain of the CFTR protein. In some embodiments, the editing target sequence comprises a mutation in a sequence of the CFTR gene that encodes an interdomain interface region of the CFTR protein.

In some embodiments, the editing target sequence comprises a mutation as compared to a wild type CFTR gene, wherein the mutation is in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, or exon 27 of the CFTR gene. In some embodiments, the editing target sequence comprises a mutation as compared to a wild type CFTR gene, wherein the mutation is in exon 1, exon 2, exon 3, exon 4, exon 5, exon 6, exon 7, exon 8, exon 9, exon 10, exon 12, exon 13, exon 14, exon 15, exon 16, exon 17, exon 18, exon 19, exon 20, exon 21, exon 22, exon 23, exon 24, exon 25, exon 26, or exon 27 of the CFTR gene. Accordingly, in some embodiments, the editing template comprises one or more intended nucleotide edits compared to the target CFTR gene, wherein incorporation of the one or more intended nucleotide edits corrects the mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, or exon 27 of the CFTR gene compared to a wild type CFTR gene. In some embodiments, the editing template comprises a sequence of a wild type CFTR gene. In some embodiments, the editing target sequence is in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, or exon 27 of the CFTR gene.

In some embodiments, the editing target sequence comprises a mutation as compared to a wild type CFTR gene, wherein the mutation is a nucleotide insertion, a nucleotide deletion, a nucleotide substitution, two or more nucleotide substitutions, or any combination thereof. In some embodiments, the mutation results in a premature stop codon in a mRNA encoded by the CFTR gene. In some embodiments, the mutation results in an amino acid alteration in the CFTR protein encoded by the CFTR gene. In some embodiments, the mutation results in an amino acid substitution in the CFTR protein encoded by the CFTR gene. In some embodiments, the mutation results in a truncated CFTR polypeptide encoded by the CFTR gene as compared to a wild type CFTR polypeptide. In some embodiments, the mutation results in an aberrant CFTR polypeptide encoded by the CFTR gene. In some embodiments, the mutation results in a CFTR polypeptide encoded by the CFTR gene that has reduced biological activity as compared to a wild type CFTR polypeptide. In some embodiments, the mutation results in a CFTR polypeptide encoded by the CFTR gene that has abolished biological activity as compared to a wild type CFTR polypeptide. In some embodiments, the editing template of a PEgRNA comprises one or more intended nucleotide edits compared to the sequence on the target strand of the target CFTR gene. In some embodiments, the editing template encodes a single stranded DNA that comprises one or more intended nucleotide edits compared to the editing target sequence. In some embodiments, the single stranded DNA replaces the editing target sequence by prime editing, thereby incorporating the one or more intended nucleotide edits. In some embodiments, incorporation of the one or more intended nucleotide edits corrects the mutation in the editing target sequence to wild type nucleotides at corresponding positions in the target CFTR gene. As used herein, “correcting” a mutation means restoring a wild type sequence at the place of the mutation in the double stranded target DNA e.g. target gene, by prime editing. In some embodiments, incorporation of the one or more intended nucleotide edits results in expression of a functional CFTR protein. For example, in some embodiments, incorporation of the one or more intended nucleotide edits results in a nucleotide substitution, insertion, or deletion that results in a codon that encodes a wild type amino acid as compared to a wild type CFTR polypeptide, while the codon is not the same as the wild type nucleotide at the corresponding position.

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a DNA nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired “lower stem” adjacent to the spacer sequence and a base paired “upper stem” following the lower stem, where the lower stem and upper stem may be connected by a “bulge” comprising unpaired RNAs. The gRNA core may further comprise a “nexus” distal from the spacer sequence, followed by a hairpin structure, e.g., at the 3′ end, as exemplified in FIG. 3. In some embodiments, the gRNA core comprises modified nucleotides as compared to a wild type gRNA core in the lower stem, upper stem, and/or the hairpin. For example, nucleotides in the lower stem, upper stem, an/or the hairpin regions may be modified, deleted, or replaced. In some embodiments, RNA nucleotides in the lower stem, upper stem, an/or the hairpin regions may be replaced with one or more DNA sequences. In some embodiments, the gRNA core comprises unmodified or wild type RNA sequences in the nexus and/or the bulge regions. In some embodiments, the gRNA core does not include long stretches of A-T pairs, for example, a GUUUU-AAAAC pairing element. In some embodiments, a prime editing system comprises a prime editor and a PEgRNA, wherein the prime editor comprises a SpCas9 nickase variant thereof, and the gRNA core of the PEgRNA comprises the sequence: GUUUUAGAGCUAGAAAUAGCAAGUUAAAAUAAGGCUAGUCCGUUAUCAACUUGAAAAAG UGGCACCGAGUCGGUGC (SEQ ID NO: 16) GUUUGAGAGCUAGAAAUAGCAAGUUUAAAUAAGGCUAGUCCGUUAUCAACUUGAAAAAG UGGGACCGAGUCGGUCC (SEQ ID NO: 17), or GUUUAAGAGCUAUGCUGGAAACAGCAUAGCAAGUUUAAAUAAGGCUAGUCCGUUAUCAA CUUGAAAAAGUGGCACCGAGUCGGUGC (SEQ ID NO: 18). In some embodiments, the gRNA core comprises the sequence of SEQ ID NO: 16. Any gRNA core sequences known in the art are also contemplated in the prime editing compositions described herein.

A PEgRNA may also comprise optional modifiers, e.g., 3′ end modifier region and/or an 5′ end modifier region. In some embodiments, a PEgRNA comprises at least one nucleotide that is not part of a spacer, a gRNA core, or an extension arm. The optional sequence modifiers could be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

In some embodiments, a prime editing system or composition further comprises a nick guide polynucleotide, such as a nick guide RNA (ngRNA). In some embodiments, a ngRNA comprises a spacer (referred to as a ngRNA spacer or ng spacer) and a gRNA core, wherein the spacer of the ngRNA comprises a region of complementarity to the edit strand, and wherein the gRNA core can interact with a Cas, e.g., Cas9, of a prime editor. Without wishing to be bound by any particular theory, an ngRNA may bind to the edit strand and direct Cas nickase to generate a nick on the non-edit strand (or target strand). In some embodiments, the nick on the non-edit strand directs endogenous DNA repair machinery to use the edit strand as a template for repair of the non-edit strand, which may increase efficiency of prime editing. In some embodiments, the non-edit strand is nicked by a prime editor localized to the non-edit strand by the ngRNA. Accordingly, also provided herein are PEgRNA systems comprising at least one PEgRNA and at least one ngRNA.

A prime editing system comprising a PEgRNA (or one or more polynucleotide encoding the PEgRNA) and a prime editor protein (or one or more polynucleotides encoding the prime editor), may be referred to as a PE2 prime editing system and the corresponding editing approach referred to as PE2 approach or PE2 strategy. A PE2 system does not contain a ngRNA. A prime editing system comprising a PEgRNA (or one or more polynucleotide encoding the PEgRNA), a prime editor protein (or one or more polynucleotides encoding the prime editor), and a ngRNA (or one or more polynucleotides encoding the ngRNA) may be referred to as a “PE3” prime editing system. In some embodiments, an ngRNA spacer sequence is complementary to a portion of the edit strand that includes the intended nucleotide edit and may hybridize with the edit strand only after the edit has been incorporated on the edit strand. Such ngRNA may be referred to a “PE3b” ngRNA, and the prime editing system a PE3b prime editing system.

In some embodiments, the ng search target sequence is located on the non-target strand, within 10 base pairs to 100 base pairs of an intended nucleotide edit incorporated by the PEgRNA on the edit strand. In some embodiments, the ng target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the PEgRNA on the edit strand. In some embodiments, the 5′ ends of the ng search target sequence and the PEgRNA search target sequence are within 1, 2, 3, 4, 5, 6, 7, 8, 9, or 10 bp apart from each other. In some embodiments, the 5′ ends of the ng search target sequence and the PEgRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp apart from each other.

In some embodiments, an ng spacer sequence is complementary to, and may hybridize with the second search target sequence only after an intended nucleotide edit has been incorporated on the edit strand, by the editing template of a PEgRNA. In some embodiments, such a prime editing system maybe referred to as a “PE3b” prime editing system or composition. In some embodiments, the ngRNA comprises a spacer sequence that matches only the edit strand after incorporation of the nucleotide edits, but not the endogenous target gene sequence on the edit strand. Accordingly, in some embodiments, an intended nucleotide edit is incorporated within the ng search target sequence. In some embodiments, the intended nucleotide edit is incorporated within about 1-10 nucleotides of the position corresponding to the PAM of the ng search target sequence.

The gRNA core of a PEgRNA or ngRNA can be any gRNA scaffold sequence that is capable of interacting with a Cas protein that recognizes the corresponding PAM of the PEgRNA or ngRNA. In some embodiments, gRNA core of a PEgRNA or a ngRNA comprises a sequence selected from SEQ ID Nos: 16-18.

Exemplary sequences for ngRNA spacer sequences are provided in Table 3D.

In some embodiments, the editing target sequence is in exon 3 of the CFTR gene. In some embodiments, the editing target sequence comprises a mutation as compared to a wild type CFTR gene.

In some embodiments, the editing target sequence or the complementary sequence on the CFTR gene thereof comprises a mutation that encodes an amino acid substitution G85E, a G85V, a F87I, a L88*, and/or a Y84* relative to a wild type CFTR polypeptide set forth in SEQ ID NO: 19, wherein * refers to a non-sense mutation, and wherein the editing template encodes an intended nucleotide edit that corrects the mutation. In some embodiments, the editing target sequence or the complementary sequence on the CFTR gene thereof comprises a G→→A mutation (c.254G>A) at position 117,509,123, a duplication of a T (c.248dupT) at position 117,509,117, a T→→A mutation (c.252T>A) at position 117,509,121, a G→→T mutation (c.254G>T) at position 117,509,123, a T→→A mutation (c.259T>A) at position 117,509,128, a deletion of nucleotides TT (c.262_263delTT) at position 117,509,131, a T→→G mutation (c.263T>G) at position 117,509,132, a T→→A mutation (c.263T>A) at position 117,509,132, and/or a deletion of ATATT (c.264_268delATATT) at position 117,509,133 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide. In some embodiments, the editing template comprises a wild type CFTR gene sequence. Accordingly, in some embodiments, the newly synthesized single stranded DNA encoded by the editing template is capable of correcting the c.254G>A mutation, the c.248dupT mutation, the c.252T>A mutation, the c.254G>T mutation, the c.259T>A mutation, the c.262_263delTT mutation, the c.263T>G mutation, the c.263T>A mutation, and/or the c.264_268delATATT mutation in the target CFTR gene. In some embodiments, the newly synthesized single stranded DNA encoded by the editing template is capable of correcting all of the c.254G>A mutation, the c.248dupT mutation, the c.252T>A mutation, the c.254G>T mutation, the c.259T>A mutation, the c.262_263delTT mutation, the c.263T>G mutation, the c.263T>A mutation, and the c.264_268delATATT mutation in the target CFTR gene. In some embodiments, two or more of the c.254G>A mutation, the c.248dupT mutation, the c.252T>A mutation, the c.254G>T mutation, the c.259T>A mutation, the c.262_263delTT mutation, the c.263T>G mutation, the c.263T>A mutation, and the c.264_268delATATT mutation are located on the same CFTR gene. In some embodiments, the newly synthesized single stranded DNA encoded by the editing template is capable of correcting the c.254G>A mutation, the c.248dupT mutation, the c.252T>A mutation, the c.254G>T mutation, the c.259T>A mutation, the c.262_263delTT mutation, the c.263T>G mutation, the c.263T>A mutation, and the c.264_268delATATT mutation that are located on different CFTR genes. For example, in some embodiments, a subject may comprise two copies of CFTR genes, each comprising a different mutation selected from the group consisting of the c.254G>A mutation, the c.248dupT mutation, the c.252T>A mutation, the c.254G>T mutation, the c.259T>A mutation, the c.262_263delTT mutation, the c.263T>G mutation, the c.263T>A mutation, and the c.264_268delATATT mutation, all of which may be corrected by an editing template of a single PEgRNA. In some embodiments, the editing template is capable of correcting one or more, or all of the c.254G>A mutation, the c.248dupT mutation, the c.252T>A mutation, the c.254G>T mutation, the c.259T>A mutation, the c.262_263delTT mutation, the c.263T>G mutation, the c.263T>A mutation, and the c.264_268delATATT mutation that are in different subjects, wherein each subject may have one or more of the mutations.

In some embodiments, the PEgRNA comprises a spacer comprising a sequence selected from the any of the sequences disclosed in Table 3A. In some embodiments, the PEgRNA comprises a PBS comprising a sequence selected from any of the sequences disclosed in Table 3B. In some embodiments, the PEgRNA comprises an editing template comprising a sequence selected from any of the sequences disclosed in Table 3C. In some embodiments, the prime editing composition further comprises a ngRNA that comprises a spacer comprising a sequence selected from any of the sequences disclosed in Table 3D.

In some embodiments, the PEgRNA comprises a spacer comprising a sequence selected from the group consisting of SEQ ID Nos: A1-A864 in Table 3A. In some embodiments, the PEgRNA comprises a PBS comprising a sequence selected from the group consisting of SEQ ID Nos: B1-B3617 in Table 3B. In some embodiments, the PEgRNA comprises an editing template comprising a sequence selected from the group consisting of SEQ ID Nos: C1-C15607 in Table 3C. In some embodiments, the prime editing composition further comprises a ngRNA that comprises a spacer comprising a sequence selected from the group consisting of ngRNA SEQ ID Nos: D1-D1998 in Table 3D.

In some embodiments, the PEgRNA comprises a spacer comprising a sequence selected from the group consisting of SEQ ID NOs. 349-1212 of Table 3A. In some embodiments, the PEgRNA comprises a PBS comprising a sequence selected from the group consisting of SEQ ID Nos: 1213-4026 of Table 3B. In some embodiments, the PEgRNA comprises an editing template comprising a sequence selected from the group consisting of SEQ ID Nos: 4027-19633 of Table 3C. In some embodiments, the prime editing composition further comprises a ngRNA that comprises a spacer comprising a sequence selected from the group consisting of SEQ ID Nos: 19634-21631 of Table 3D.

In some embodiments, the PEgRNA comprises a spacer comprising a sequence selected from the group consisting of SEQ Identifiers: A1-A2 in Table 3A. In some embodiments, the PEgRNA comprises a spacer comprising a sequence selected from the group consisting of SEQ ID Nos: 349-350 in Table 3A. In some embodiments, the PEgRNA comprises a PBS comprising a sequence selected from the group consisting of SEQ Identifiers: B1-B9 in Table 3B. In some embodiments, the PEgRNA comprises a PBS comprising a sequence selected from the group consisting of SEQ ID Nos: 1213-1219 in Table 3B. In some embodiments, the PEgRNA comprises a PBS comprising a sequence selected from the group consisting of sequences TGTACATT and TGTACATTC. In some embodiments, the PEgRNA comprises an editing template comprising a sequence selected from the group consisting of SEQ Identifiers: C1-C12 in Table 3C. In some embodiments, the PEgRNA comprises an editing template comprising a sequence selected from the group consisting of SEQ ID Nos: 4027-4038 in Table 3C. In some embodiments, the prime editing composition further comprises a ngRNA that comprises a spacer comprising a sequence selected from the group consisting of ngRNA SEQ ID Identifiers: D1-D8 in Table 3D. In some embodiments, the prime editing composition further comprises a ngRNA that comprises a spacer comprising a sequence selected from the group consisting of ngRNA SEQ ID Nos: 19634-19641 in Table 3D.

In some embodiments, the PEgRNA comprises a spacer comprising a sequence selected from the group consisting of SEQ Identifiers: A1-A2 in Table 3A. In some embodiments, the PEgRNA comprises a spacer comprising a sequence selected from the group consisting of SEQ ID Nos: 349-350 in Table 3A. In some embodiments, the PEgRNA comprises a PBS comprising a sequence selected from the group consisting of SEQ Identifiers: B1-B9 in Table 3B. In some embodiments, the PEgRNA comprises a PBS comprising a sequence selected from the group consisting of SEQ ID Nos: 1213-1219 in Table 3B. In some embodiments, the PEgRNA comprises a PBS comprising a sequence selected from the group consisting of sequences TGTACATT and TGTACATTC. In some embodiments, the PEgRNA comprises an editing template comprising a sequence selected from the group consisting of SEQ Identifiers: C1-C12 in Table 3C. In some embodiments, the PEgRNA comprises an editing template comprising a sequence selected from the group consisting of SEQ ID Nos: 4027-4038 in Table 3C. In some embodiments, the prime editing composition further comprises a ngRNA that comprises a spacer comprising a sequence selected from the group consisting of ngRNA SEQ ID Identifiers: D1-D8 in Table 3D. In some embodiments, the prime editing composition further comprises a ngRNA that comprises a spacer comprising a sequence selected from the group consisting of ngRNA SEQ ID Nos: 19634-19641 in Table 3D.

In some embodiments, the PEgRNA comprises a spacer comprising a sequence selected from the group consisting of SEQ Identifiers: A3-A4 in Table 3A. In some embodiments, the PEgRNA comprises a spacer comprising a sequence selected from the group consisting of SEQ ID Nos: 351-352 in Table 3A. In some embodiments, the PEgRNA comprises a PBS comprising a sequence selected from the group consisting of SEQ Identifiers: B10-B18 in Table 3B. In some embodiments, the PEgRNA comprises a PBS comprising a sequence selected from the group consisting of SEQ ID Nos: 1220-1226 in Table 3B. In some embodiments, the PEgRNA comprises a PBS comprising a sequence selected from the group consisting of sequences AAGGGCAT and AAGGGCATT. In some embodiments, the PEgRNA comprises an editing template comprising a sequence selected from the group consisting of SEQ ID Identifiers: C13-C17 in Table 3C. In some embodiments, the PEgRNA comprises an editing template comprising a sequence selected from the group consisting of SEQ ID Nos: 4039-4043 in Table 3C. In some embodiments, the prime editing composition further comprises a ngRNA that comprises a spacer comprising a sequence selected from the group consisting of SEQ Identifiers: D9-D32 in Table 3D. In some embodiments, the prime editing composition further comprises a ngRNA that comprises a spacer comprising a sequence selected from the group consisting of SEQ ID Nos: 19642-19665 in Table 3D.

In some embodiments, the PEgRNA comprises a spacer comprising the sequence of SEQ ID Identifiers: A5 in Table 3A. In some embodiments, the PEgRNA comprises a spacer comprising the sequence of SEQ ID Nos: 353 in Table 3A. In some embodiments, the PEgRNA comprises a PBS comprising a sequence selected from the group consisting of SEQ Identifiers: B19-B27 in Table 3B. In some embodiments, the PEgRNA comprises a PBS comprising a sequence selected from the group consisting of SEQ ID Nos: 1227-1233 in Table 3B. In some embodiments, the PEgRNA comprises a PBS comprising a sequence selected from sequences ATCTCCAG and ATCTCCAGA. In some embodiments, the PEgRNA comprises an editing template comprising a sequence selected from the group consisting of SEQ Identifiers: C18-C44 in Table 3C. In some embodiments, the PEgRNA comprises an editing template comprising a sequence selected from the group consisting of SEQ ID Nos: 4044-4070 in Table 3C. In some embodiments, the prime editing composition further comprises a ngRNA that comprises a spacer comprising a sequence selected from the group consisting of SEQ Identifiers: D11-D27 and D33-D38 in Table 3D. In some embodiments, the prime editing composition further comprises a ngRNA that comprises a spacer comprising a sequence selected from the group consisting of SEQ ID Nos: 19644-19660 and 19666-19671 in Table 3D.

In some embodiments, the PEgRNA comprises a spacer comprising a sequence selected from the group consisting of SEQ Identifiers: A6-A7 in Table 3A. In some embodiments, the PEgRNA comprises a spacer comprising a sequence selected from the group consisting of SEQ ID Nos: 354-355 in Table 3A. In some embodiments, the PEgRNA comprises a PBS comprising a sequence selected from the group consisting of SEQ Identifiers: B28-B36 in Table 3B. In some embodiments, the PEgRNA comprises a PBS comprising a sequence selected from the group consisting of SEQ ID Nos: 1234-1240 in Table 3B. In some embodiments, the PEgRNA comprises a PBS comprising a sequence selected from the group consisting of sequences TTGTACAT and TTGTACATT. In some embodiments, the PEgRNA comprises an editing template comprising a sequence selected from the group consisting of SEQ Identifiers: C45-C51 in Table 3C. In some embodiments, the PEgRNA comprises an editing template comprising a sequence selected from the group consisting of SEQ ID Nos: 4071-4077 in Table 3C. In some embodiments, the prime editing composition further comprises a ngRNA that comprises a spacer comprising a sequence selected from the group consisting of SEQ Identifiers: D39-D62 in Table 3D. In some embodiments, the prime editing composition further comprises a ngRNA that comprises a spacer comprising a sequence selected from the group consisting of SEQ ID Nos: 19672-19695 in Table 3D.

In some embodiments, the PEgRNA comprises a spacer comprising a sequence selected from the group consisting of SEQ Identifiers: A8-A10 in Table 3A. In some embodiments, the PEgRNA comprises a spacer comprising a sequence selected from the group consisting of SEQ ID Nos: 356-358 in Table 3A. In some embodiments, the PEgRNA comprises a PBS comprising a sequence selected from the group consisting of SEQ Identifiers: B37-B45 in Table 3B. In some embodiments, the PEgRNA comprises a PBS comprising a sequence selected from the group consisting of SEQ ID Nos: 1241-1247 in Table 3B. In some embodiments, the PEgRNA comprises a PBS comprising a sequence selected from the group consisting of sequences GCATTAAT and GCATTAATG. In some embodiments, the PEgRNA comprises an editing template comprising the sequence of SEQ Identifiers: C52 in Table 3C. In some embodiments, the PEgRNA comprises an editing template comprising the sequence of SEQ ID Nos: 4078 in Table 3C. In some embodiments, the prime editing composition further comprises a ngRNA that comprises a spacer comprising a sequence selected from the group consisting of SEQ Identifiers: D17 and D63-D70 in Table 3D. In some embodiments, the prime editing composition further comprises a ngRNA that comprises a spacer comprising a sequence selected from the group consisting of SEQ ID Nos: 19650 and 19696-19703 in Table 3D.

In some embodiments, the PEgRNA comprises a spacer comprising a sequence selected from the group consisting of SEQ Identifiers: A11-A12 in Table 3A. In some embodiments, the PEgRNA comprises a spacer comprising a sequence selected from the group consisting of SEQ ID Nos: 359-360 in Table 3A. In some embodiments, the PEgRNA comprises a PBS comprising a sequence selected from the group consisting of SEQ Identifiers: B46-B54 in Table 3B. In some embodiments, the PEgRNA comprises a PBS comprising a sequence selected from the group consisting of SEQ ID Nos: 1248-1254 in Table 3B. In some embodiments, the PEgRNA comprises a PBS comprising a sequence selected from the group consisting of sequences ATATTTAG and ATATTTAGG. In some embodiments, the PEgRNA comprises an editing template comprising a sequence selected from the group consisting of SEQ Identifiers: C53-C112 in Table 3C. In some embodiments, the PEgRNA comprises an editing template comprising a sequence selected from the group consisting of SEQ ID Nos: 4079-4138 in Table 3C. In some embodiments, the prime editing composition further comprises a ngRNA that comprises a spacer comprising a sequence selected from the group consisting of SEQ Identifiers: D1-D7 and D71-D78 in Table 3D. In some embodiments, the prime editing composition further comprises a ngRNA that comprises a spacer comprising a sequence selected from the group consisting of SEQ ID Nos: 19634-19640 and 19704-19711 in Table 3D.

In some embodiments, the PEgRNA comprises a spacer comprising a sequence selected from the group consisting of SEQ Identifiers: A13-A14 in Table 3A. In some embodiments, the PEgRNA comprises a spacer comprising a sequence selected from the group consisting of SEQ ID Nos: A13-A14 in Table 3A. In some embodiments, the PEgRNA comprises a PBS comprising a sequence selected from the group consisting of SEQ Identifiers: B55-B63 in Table 3B. In some embodiments, the PEgRNA comprises a PBS comprising a sequence selected from the group consisting of SEQ ID Nos: 1255-1261 in Table 3B. In some embodiments, the PEgRNA comprises a PBS comprising a sequence selected from the group consisting of sequences: AAAACATC and AAAACATCG. In some embodiments, the PEgRNA comprises an editing template comprising a sequence selected from the group consisting of SEQ Identifiers: C113-C129 in Table 3C. In some embodiments, the PEgRNA comprises an editing template comprising a sequence selected from the group consisting of SEQ ID Nos: 4139-4155 in Table 3C. In some embodiments, the prime editing composition further comprises a ngRNA that comprises a spacer comprising a sequence selected from the group consisting of SEQ Identifiers: D9-D27, D33-D37, and D79-D83 in Table 3D. In some embodiments, the prime editing composition further comprises a ngRNA that comprises a spacer comprising a sequence selected from the group consisting of SEQ ID Nos: 19642-19660, 19666-19670, and 19712-19716 in Table 3D.

In some embodiments, the PEgRNA comprises a spacer comprising a sequence selected from the group consisting of SEQ Identifiers: A15-A16 in Table 3A. In some embodiments, the PEgRNA comprises a spacer comprising a sequence selected from the group consisting of SEQ ID Nos: 363-364 in Table 3A. In some embodiments, the PEgRNA comprises a PBS comprising a sequence selected from the group consisting of SEQ Identifiers: B64-B72 in Table 3B. In some embodiments, the PEgRNA comprises a PBS comprising a sequence selected from the group consisting of SEQ ID Nos: 1262-1268 in Table 3B. In some embodiments, the PEgRNA comprises a PBS comprising a sequence selected from the group consisting of sequences: TATATTTA and TATATTTAG. In some embodiments, the PEgRNA comprises an editing template comprising a sequence selected from the group consisting of SEQ ID Nos: C130-C160 in Table 3C. In some embodiments, the PEgRNA comprises an editing template comprising a sequence selected from the group consisting of SEQ ID Nos: 4156-4186 in Table 3C. In some embodiments, the prime editing composition further comprises a ngRNA that comprises a spacer comprising a sequence selected from the group consisting of SEQ ID Nos: D39-D61, and D84-D91 in Table 3D. In some embodiments, the prime editing composition further comprises a ngRNA that comprises a spacer comprising a sequence selected from the group consisting of SEQ ID Nos: 19672-19695, and 19717-19724 in Table 3D.

In some embodiments, the PEgRNA comprises a spacer comprising a sequence selected from the group consisting of SEQ Identifiers: A17-A18 in Table 3A. In some embodiments, the PEgRNA comprises a spacer comprising a sequence selected from the group consisting of SEQ ID Nos: 365-366 in Table 3A. In some embodiments, the PEgRNA comprises a PBS comprising a sequence selected from the group consisting of SEQ Identifiers: B73-B81 in Table 3B. In some embodiments, the PEgRNA comprises a PBS comprising a sequence selected from the group consisting of SEQ ID Nos: 1269-1275 in Table 3B. In some embodiments, the PEgRNA comprises a PBS comprising a sequence selected from the group consisting of sequences: AAAAACAT and AAAAACATC. In some embodiments, the PEgRNA comprises an editing template comprising a sequence selected from the group consisting of SEQ ID Nos: C161-C178 in Table 3C. In some embodiments, the PEgRNA comprises an editing template comprising a sequence selected from the group consisting of SEQ ID Nos: 4187-4204 in Table 3C. In some embodiments, the prime editing composition further comprises a ngRNA that comprises a spacer comprising a sequence selected from the group consisting of SEQ ID Nos: D9-D37 and D92 in Table 3D. In some embodiments, the prime editing composition further comprises a ngRNA that comprises a spacer comprising a sequence selected from the group consisting of SEQ ID Nos: 19642-19670 and 19725 in Table 3D.

In some embodiments, the PEgRNA comprises a spacer comprising a sequence selected from the group consisting of SEQ Identifiers: A19-A21 in Table 3A. In some embodiments, the PEgRNA comprises a spacer comprising a sequence selected from the group consisting of SEQ ID Nos: 367-369 in Table 3A. In some embodiments, the PEgRNA comprises a PBS comprising a sequence selected from the group consisting of SEQ Identifiers: B82-B90 in Table 3B. In some embodiments, the PEgRNA comprises a PBS comprising a sequence selected from the group consisting of SEQ ID Nos: 1276-1282 in Table 3B. In some embodiments, the PEgRNA comprises a PBS comprising a sequence selected from the group consisting of sequences: AAACATCG and AAACATCGC. In some embodiments, the PEgRNA comprises an editing template comprising a sequence selected from the group consisting of SEQ ID Nos: C179-C194 in Table 3C. In some embodiments, the PEgRNA comprises an editing template comprising a sequence selected from the group consisting of SEQ ID Nos: 4205-4220 in Table 3C. In some embodiments, the prime editing composition further comprises a ngRNA that comprises a spacer comprising a sequence selected from the group consisting of SEQ ID Nos: D17, D35, D63-D70 and D93-D97 in Table 3D. In some embodiments, the prime editing composition further comprises a ngRNA that comprises a spacer comprising a sequence selected from the group consisting of SEQ ID Nos: 19650, 19668, 19696-19703 and 19726-19730 in Table 3D.

In some embodiments, the PEgRNA comprises a spacer comprising a sequence selected from the group consisting of SEQ Identifiers: A22-A23 in Table 3A. In some embodiments, the PEgRNA comprises a spacer comprising a sequence selected from the group consisting of SEQ ID Nos: 370-371 in Table 3A. In some embodiments, the PEgRNA comprises a PBS comprising a sequence selected from the group consisting of SEQ Identifiers: B91-B99 in Table 3B. In some embodiments, the PEgRNA comprises a PBS comprising a sequence selected from the group consisting of SEQ ID Nos: 1283-1289 in Table 3B. In some embodiments, the PEgRNA comprises a PBS comprising a sequence selected from the group consisting of sequences: GGCATTAA and GGCATTAAT. In some embodiments, the PEgRNA comprises an editing template comprising a sequence selected from the group consisting of SEQ ID Nos: C195-C198 in Table 3C. In some embodiments, the PEgRNA comprises an editing template comprising a sequence selected from the group consisting of SEQ ID Nos: 4221-4224 in Table 3C. In some embodiments, the prime editing composition further comprises a ngRNA that comprises a spacer comprising a sequence selected from the group consisting of SEQ ID Nos: D9-D27, D79-D81 and D98-D99 in Table 3D. In some embodiments, the prime editing composition further comprises a ngRNA that comprises a spacer comprising a sequence selected from the group consisting of SEQ ID Nos: 19642-19660, 19712-19714 and 19731-19732 in Table 3D.

In some embodiments, the PEgRNA comprises a spacer comprising the sequence of SEQ Identifiers: A24 in Table 3A. In some embodiments, the PEgRNA comprises a spacer comprising the sequence of SEQ ID Nos: 372 in Table 3A. In some embodiments, the PEgRNA comprises a PBS comprising a sequence selected from the group consisting of SEQ Identifiers: B100-B108 in Table 3B. In some embodiments, the PEgRNA comprises a PBS comprising a sequence selected from the group consisting of SEQ ID Nos: 1290-1296 in Table 3B. In some embodiments, the PEgRNA comprises a PBS comprising a sequence selected from the group consisting of sequences: GGGCATTA and GGGCATTAA. In some embodiments, the PEgRNA comprises an editing template comprising a sequence selected from the group consisting of SEQ identifiers: C199-C201 in Table 3C. In some embodiments, the PEgRNA comprises an editing template comprising a sequence selected from the group consisting of SEQ ID Nos: 4225-4227 in Table 3C. In some embodiments, the prime editing composition further comprises a ngRNA that comprises a spacer comprising a sequence selected from the group consisting of SEQ identifiers: D9-D27 in Table 3D. In some embodiments, the prime editing composition further comprises a ngRNA that comprises a spacer comprising a sequence selected from the group consisting of SEQ ID Nos: 19642-19660 in Table 3D.

In some embodiments, the PEgRNA comprises a spacer comprising a sequence selected from the group consisting of SEQ Identifiers: A25-A26 in Table 3A. In some embodiments, the PEgRNA comprises a spacer comprising a sequence selected from the group consisting of SEQ ID Nos: 373-374 in Table 3A. In some embodiments, the PEgRNA comprises a PBS comprising a sequence selected from the group consisting of SEQ identifiers: B109-B117 in Table 3B. In some embodiments, the PEgRNA comprises a PBS comprising a sequence selected from the group consisting of SEQ ID Nos: 1297-1303 in Table 3B. In some embodiments, the PEgRNA comprises a PBS comprising a sequence selected from the group consisting of sequences: AATCTTTT and AATCTTTTT. In some embodiments, the PEgRNA comprises an editing template comprising a sequence selected from the group consisting of SEQ identifiers: C202-C263 in Table 3C. In some embodiments, the PEgRNA comprises an editing template comprising a sequence selected from the group consisting of SEQ ID Nos: 4228-4289 in Table 3C. In some embodiments, the prime editing composition further comprises a ngRNA that comprises a spacer comprising a sequence selected from the group consisting of SEQ identifiers: D1-D7, D73-D74, D77-D78, and D100 in Table 3D. In some embodiments, the prime editing composition further comprises a ngRNA that comprises a spacer comprising a sequence selected from the group consisting of SEQ ID Nos: 19634-19640, 19706-19707, 19710-19711, and 19733 in Table 3D.

In some embodiments, the PEgRNA comprises a spacer comprising a sequence selected from the group consisting of SEQ Identifiers: A27-A28 in Table 3A. In some embodiments, the PEgRNA comprises a spacer comprising a sequence selected from the group consisting of SEQ ID Nos: 375-376 in Table 3A. In some embodiments, the PEgRNA comprises a PBS comprising a sequence selected from the group consisting of SEQ identifiers: B118-B126 in Table 3B. In some embodiments, the PEgRNA comprises a PBS comprising a sequence selected from the group consisting of SEQ ID Nos: 1304-B126 in Table 3B. In some embodiments, the PEgRNA comprises a PBS comprising a sequence selected from the group consisting of sequences: GTACATTC and GTACATTCA. In some embodiments, the PEgRNA comprises an editing template comprising a sequence selected from the group consisting of SEQ identifiers: C264-C268 in Table 3C. In some embodiments, the PEgRNA comprises an editing template comprising a sequence selected from the group consisting of SEQ ID Nos: 4290-4294 in Table 3C. In some embodiments, the prime editing composition further comprises a ngRNA that comprises a spacer comprising a sequence selected from the group consisting of SEQ identifiers: D39-D62 in Table 3D. In some embodiments, the prime editing composition further comprises a ngRNA that comprises a spacer comprising a sequence selected from the group consisting of SEQ ID Nos: 19672-19695 in Table 3D.

In some embodiments, the PEgRNA comprises a spacer comprising the sequence of SEQ Identifiers: A29 in Table 3A. In some embodiments, the PEgRNA comprises a spacer comprising the sequence of SEQ ID Nos: 377 in Table 3A. In some embodiments, the PEgRNA comprises a PBS comprising a sequence selected from the group consisting of SEQ identifiers: B127-B135 in Table 3B. In some embodiments, the PEgRNA comprises a PBS comprising a sequence selected from the group consisting of SEQ ID Nos: 1311-1317 in Table 3B. In some embodiments, the PEgRNA comprises a PBS comprising a sequence selected from the group consisting of sequences: CCGAAGGG and CCGAAGGGC. In some embodiments, the PEgRNA comprises an editing template comprising a sequence selected from the group consisting of SEQ identifiers: C269-C276 in Table 3C. In some embodiments, the PEgRNA comprises an editing template comprising a sequence selected from the group consisting of SEQ ID Nos: 4295-4302 in Table 3C. In some embodiments, the prime editing composition further comprises a ngRNA that comprises a spacer comprising a sequence selected from the group consisting of SEQ identifiers: D9-D27 and D33-D35 in Table 3D. In some embodiments, the prime editing composition further comprises a ngRNA that comprises a spacer comprising a sequence selected from the group consisting of SEQ ID Nos: 19642-19660 and 19666-19668 in Table 3D.

In some embodiments, the PEgRNA comprises a spacer comprising a sequence selected from the group consisting of SEQ Identifiers: A30-A31 in Table 3A. In some embodiments, the PEgRNA comprises a spacer comprising a sequence selected from the group consisting of SEQ ID Nos: 378-379 in Table 3A. In some embodiments, the PEgRNA comprises a PBS comprising a sequence selected from the group consisting of SEQ identifiers: B136-B144 in Table 3B. In some embodiments, the PEgRNA comprises a PBS comprising a sequence selected from the group consisting of SEQ ID Nos: 1318-1324 in Table 3B. In some embodiments, the PEgRNA comprises a PBS comprising a sequence selected from the group consisting of sequences: GAAAAAAC and GAAAAAACA. In some embodiments, the PEgRNA comprises an editing template comprising a sequence selected from the group consisting of SEQ identifiers: C277-C296 in Table 3C. In some embodiments, the PEgRNA comprises an editing template comprising a sequence selected from the group consisting of SEQ ID Nos: 4303-4322 in Table 3C. In some embodiments, the prime editing composition further comprises a ngRNA that comprises a spacer comprising a sequence selected from the group consisting of SEQ identifiers: D9-D37, and D92 in Table 3D. In some embodiments, the prime editing composition further comprises a ngRNA that comprises a spacer comprising a sequence selected from the group consisting of SEQ ID Nos: 19642-19670 and 19725 in Table 3D.

In some embodiments, the PEgRNA comprises a spacer comprising a sequence selected from the group consisting of SEQ Identifiers: A32-A33 in Table 3A. In some embodiments, the PEgRNA comprises a spacer comprising a sequence selected from the group consisting of SEQ ID Nos: 380-381 in Table 3A. In some embodiments, the PEgRNA comprises a PBS comprising a sequence selected from the group consisting of SEQ identifiers: B145-B153 in Table 3B. In some embodiments, the PEgRNA comprises a PBS comprising a sequence selected from the group consisting of SEQ ID Nos: 1325-1331 in Table 3B. In some embodiments, the PEgRNA comprises a PBS comprising a sequence selected from the group consisting of sequences: AACATAAA and AACATAAAT. In some embodiments, the PEgRNA comprises an editing template comprising a sequence selected from the group consisting of SEQ identifiers: C297-C327 in Table 3C. In some embodiments, the PEgRNA comprises an editing template comprising a sequence selected from the group consisting of SEQ ID Nos: 4323-4353 in Table 3C. In some embodiments, the prime editing composition further comprises a ngRNA that comprises a spacer comprising a sequence selected from the group consisting of SEQ identifiers: D11-D38, D92, and D101 in Table 3D. In some embodiments, the prime editing composition further comprises a ngRNA that comprises a spacer comprising a sequence selected from the group consisting of SEQ ID Nos: 19644-19671, 19725, and 19734 in Table 3D.

In some embodiments, the PEgRNA comprises a spacer comprising a sequence selected from the group consisting of SEQ Identifiers: A34-A35 in Table 3A. In some embodiments, the PEgRNA comprises a spacer comprising a sequence selected from the group consisting of SEQ ID Nos: 382-383 in Table 3A. In some embodiments, the PEgRNA comprises a PBS comprising a sequence selected from the group consisting of SEQ identifiers: B154-B162 in Table 3B. In some embodiments, the PEgRNA comprises a PBS comprising a sequence selected from the group consisting of SEQ ID Nos: 1332-1336 in Table 3B. In some embodiments, the PEgRNA comprises a PBS comprising a sequence selected from the group consisting of sequences: AAAAAACA and AAAAAACAT. In some embodiments, the PEgRNA comprises an editing template comprising a sequence selected from the group consisting of SEQ identifiers: C328-C365 in Table 3C. In some embodiments, the PEgRNA comprises an editing template comprising a sequence selected from the group consisting of SEQ ID Nos: 4354-4391 in Table 3C. In some embodiments, the prime editing composition further comprises a ngRNA that comprises a spacer comprising a sequence selected from the group consisting of SEQ identifiers: D67, D95, D102-D119 in Table 3D. In some embodiments, the prime editing composition further comprises a ngRNA that comprises a spacer comprising a sequence selected from the group consisting of SEQ ID Nos: 19700, 19728, 19735-19752 in Table 3D.

In some embodiments, the PEgRNA comprises a spacer comprising a sequence selected from the group consisting of SEQ Identifiers: A1-A2 in Table 3A. In some embodiments, the PEgRNA comprises a spacer comprising a sequence selected from the group consisting of SEQ ID Nos: 349-350 in Table 3A. In some embodiments, the PEgRNA comprises a PBS comprising a sequence selected from the group consisting of SEQ identifiers: B1-B9 in Table 3B. In some embodiments, the PEgRNA comprises a PBS comprising a sequence selected from the group consisting of SEQ ID Nos: 1213-1219 in Table 3B. In some embodiments, the PEgRNA comprises a PBS comprising a sequence selected from the group consisting of sequences: TGTACATT and TGTACATTC. In some embodiments, the PEgRNA comprises an editing template comprising a sequence selected from the group consisting of SEQ identifiers: C366-C393 in Table 3C. In some embodiments, the PEgRNA comprises an editing template comprising a sequence selected from the group consisting of SEQ ID Nos: 4392-4419 in Table 3C. In some embodiments, the prime editing composition further comprises a ngRNA that comprises a spacer comprising a sequence selected from the group consisting of SEQ identifiers: D1-D8 in Table 3D. In some embodiments, the prime editing composition further comprises a ngRNA that comprises a spacer comprising a sequence selected from the group consisting of SEQ ID Nos: 19634-19641 in Table 3D.

In some embodiments, the PEgRNA comprises a spacer comprising a sequence selected from the group consisting of SEQ Identifiers: A36-A37 in Table 3A. In some embodiments, the PEgRNA comprises a spacer comprising a sequence selected from the group consisting of SEQ ID Nos: 384-385 in Table 3A. In some embodiments, the PEgRNA comprises a PBS comprising a sequence selected from the group consisting of SEQ identifiers: B163-B171 in Table 3B. In some embodiments, the PEgRNA comprises a PBS comprising a sequence selected from the group consisting of SEQ ID Nos: 1339-1345 in Table 3B. In some embodiments, the PEgRNA comprises a PBS comprising a sequence selected from the group consisting of sequences: TGTATCAC and TGTATCACA. In some embodiments, the PEgRNA comprises an editing template comprising the sequence of SEQ identifier: C394. In some embodiments, the PEgRNA comprises an editing template comprising the sequence of SEQ ID No: 4420. In some embodiments, the prime editing composition further comprises a ngRNA that comprises a spacer comprising a sequence selected from the group consisting of SEQ ID Nos: D40-D62 in Table 3D. In some embodiments, the prime editing composition further comprises a ngRNA that comprises a spacer comprising a sequence selected from the group consisting of SEQ ID Nos: 19673-19695 in Table 3D.

In some embodiments, the PEgRNA comprises a spacer comprising a sequence selected from the group consisting of SEQ identifiers: A38-A39 in Table 3A. In some embodiments, the PEgRNA comprises a spacer comprising a sequence selected from the group consisting of SEQ ID Nos: 386-387 in Table 3A. In some embodiments, the PEgRNA comprises a PBS comprising a sequence selected from the group consisting of SEQ identifiers: B172-B180 in Table 3B. In some embodiments, the PEgRNA comprises a PBS comprising a sequence selected from the group consisting of SEQ ID Nos: 1346-1352 in Table 3B. In some embodiments, the PEgRNA comprises a PBS comprising a sequence selected from the group consisting of sequences: ATTTAGGG and ATTTAGGGG. In some embodiments, the PEgRNA comprises an editing template comprising a sequence selected from the group consisting of SEQ identifiers: C395-C425 in Table 3C. In some embodiments, the PEgRNA comprises an editing template comprising a sequence selected from the group consisting of SEQ ID Nos: 4421-4451 in Table 3C. In some embodiments, the prime editing composition further comprises a ngRNA that comprises a spacer comprising a sequence selected from the group consisting of SEQ identifiers: D1-D7 and D120-D121 in Table 3D. In some embodiments, the prime editing composition further comprises a ngRNA that comprises a spacer comprising a sequence selected from the group consisting of SEQ ID Nos: 19634-19640 and 19753-19754 in Table 3D.

In some embodiments, the PEgRNA comprises a spacer comprising the sequence of SEQ identifier: A5 in Table 3A. In some embodiments, the PEgRNA comprises a spacer comprising the sequence of SEQ ID No: 353 in Table 3A. In some embodiments, the PEgRNA comprises a PBS comprising a sequence selected from the group consisting of SEQ identifiers: B19-B27 in Table 3B. In some embodiments, the PEgRNA comprises a PBS comprising a sequence selected from the group consisting of SEQ ID Nos: 1227-1233 in Table 3B. In some embodiments, the PEgRNA comprises a PBS comprising a sequence selected from the group consisting of sequences ATCTCCAG and ATCTCCAGA. In some embodiments, the PEgRNA comprises an editing template comprising a sequence selected from the group consisting of SEQ identifiers: C426-C443 in Table 3C. In some embodiments, the prime editing composition further comprises a ngRNA that comprises a spacer comprising a sequence selected from the group consisting of SEQ identifiers: D11-D27, D33-D37, and D122 in Table 3D. In some embodiments, the PEgRNA comprises an editing template comprising a sequence selected from the group consisting of SEQ ID Nos: 4452-4469 in Table 3C. In some embodiments, the prime editing composition further comprises a ngRNA that comprises a spacer comprising a sequence selected from the group consisting of SEQ ID Nos: 19644-19660, 19666-19670, and 19755 in Table 3D.

In some embodiments, the PEgRNA comprises a spacer comprising a sequence selected from the group consisting of SEQ identifiers: A6-A7 in Table 3A. In some embodiments, the PEgRNA comprises a spacer comprising a sequence selected from the group consisting of SEQ ID Nos: 354-355 in Table 3A. In some embodiments, the PEgRNA comprises a PBS comprising a sequence selected from the group consisting of SEQ identifiers: B28-B36 in Table 3B. In some embodiments, the PEgRNA comprises a PBS comprising a sequence selected from the group consisting of SEQ ID Nos: 1234-1240 in Table 3B. In some embodiments, the PEgRNA comprises a PBS comprising a sequence selected from the group consisting of sequences: TTGTACAT and TTGTACATT. In some embodiments, the PEgRNA comprises an editing template comprising a sequence selected from the group consisting of SEQ identifiers: C444-C458 in Table 3C. In some embodiments, the prime editing composition further comprises a ngRNA that comprises a spacer comprising a sequence selected from the group consisting of SEQ identifiers: D39-D62 in Table 3D. In some embodiments, the PEgRNA comprises an editing template comprising a sequence selected from the group consisting of SEQ ID Nos: 4470-4484 in Table 3C. In some embodiments, the prime editing composition further comprises a ngRNA that comprises a spacer comprising a sequence selected from the group consisting of SEQ ID Nos: 19672-19695 in Table 3D.

In some embodiments, the PEgRNA comprises a spacer comprising a sequence selected from the group consisting of SEQ identifiers: A13-A14 in Table 3A. In some embodiments, the PEgRNA comprises a spacer comprising a sequence selected from the group consisting of SEQ ID Nos: 361-362 in Table 3A. In some embodiments, the PEgRNA comprises a PBS comprising a sequence selected from the group consisting of SEQ identifiers: B55-B63 in Table 3B. In some embodiments, the PEgRNA comprises a PBS comprising a sequence selected from the group consisting of SEQ ID Nos: 1255-1261 in Table 3B. In some embodiments, the PEgRNA comprises a PBS comprising a sequence selected from the group consisting of sequences: AAAACATC and AAAACATCG. In some embodiments, the PEgRNA comprises an editing template comprising a sequence selected from the group consisting of SEQ identifiers: C459-C466 in Table 3C. In some embodiments, the prime editing composition further comprises a ngRNA that comprises a spacer comprising a sequence selected from the group consisting of SEQ identifiers: D10-D27, D33-D37, and D79-D83 in Table 3D. In some embodiments, the PEgRNA comprises an editing template comprising a sequence selected from the group consisting of SEQ ID Nos: 4485-4492 in Table 3C. In some embodiments, the prime editing composition further comprises a ngRNA that comprises a spacer comprising a sequence selected from the group consisting of SEQ ID Nos: 19643-19660, 19666-19670, and 19712-19716 in Table 3D.

In some embodiments, the PEgRNA comprises a spacer comprising a sequence selected from the group consisting of SEQ identifiers: A40-A41 in Table 3A. In some embodiments, the PEgRNA comprises a spacer comprising a sequence selected from the group consisting of SEQ ID Nos: 388-389 in Table 3A. In some embodiments, the PEgRNA comprises a PBS comprising a sequence selected from the group consisting of SEQ identifiers: B181-B189 in Table 3B. In some embodiments, the PEgRNA comprises a PBS comprising a sequence selected from the group consisting of SEQ ID Nos: 1353-1359 in Table 3B. In some embodiments, the PEgRNA comprises a PBS comprising a sequence selected from the group consisting of GAACATAA and GAACATAAA. In some embodiments, the PEgRNA comprises an editing template comprising a sequence selected from the group consisting of SEQ identifiers: C467-C492 in Table 3C. In some embodiments, the prime editing composition further comprises a ngRNA that comprises a spacer comprising a sequence selected from the group consisting of SEQ identifiers: D11-D37, D92, and D122-D123 in Table 3D. In some embodiments, the PEgRNA comprises an editing template comprising a sequence selected from the group consisting of SEQ ID Nos: 4493-4518 in Table 3C. In some embodiments, the prime editing composition further comprises a ngRNA that comprises a spacer comprising a sequence selected from the group consisting of SEQ ID Nos: 19644-19670, 19725, and 19755-19756 in Table 3D.

In some embodiments, the PEgRNA comprises a spacer comprising a sequence selected from the group consisting of SEQ identifiers: A17-A18 in Table 3A. In some embodiments, the PEgRNA comprises a spacer comprising a sequence selected from the group consisting of SEQ ID Nos: 365-366 in Table 3A. In some embodiments, the PEgRNA comprises a PBS comprising a sequence selected from the group consisting of SEQ identifiers: B73-B81 in Table 3B. In some embodiments, the PEgRNA comprises a PBS comprising a sequence selected from the group consisting of SEQ ID Nos: 1269-1275 in Table 3B. In some embodiments, the PEgRNA comprises a PBS comprising a sequence selected from the group consisting of sequences: AAAAACAT and AAAAACATC. In some embodiments, the PEgRNA comprises an editing template comprising a sequence selected from the group consisting of SEQ identifiers: C493-C501 in Table 3C. In some embodiments, the prime editing composition further comprises a ngRNA that comprises a spacer comprising a sequence selected from the group consisting of SEQ identifiers: D10-D37, and D92 in Table 3D. In some embodiments, the PEgRNA comprises an editing template comprising a sequence selected from the group consisting of SEQ ID Nos: 4519-4527 in Table 3C. In some embodiments, the prime editing composition further comprises a ngRNA that comprises a spacer comprising a sequence selected from the group consisting of SEQ ID Nos: 19643-19670, and 19725 in Table 3D.

In some embodiments, the PEgRNA comprises a spacer comprising a sequence selected from the group consisting of SEQ identifiers: A19-A21 in Table 3A. In some embodiments, the PEgRNA comprises a spacer comprising a sequence selected from the group consisting of SEQ ID Nos: 367-369 in Table 3A. In some embodiments, the PEgRNA comprises a PBS comprising a sequence selected from the group consisting of SEQ identifiers: B82-B90 in Table 3B. In some embodiments, the PEgRNA comprises a PBS comprising a sequence selected from the group consisting of SEQ ID Nos: 1276-1282 in Table 3B. In some embodiments, the PEgRNA comprises a PBS comprising a sequence selected from the group consisting of sequences: AAACATCG and AAACATCGC. In some embodiments, the PEgRNA comprises an editing template comprising a sequence selected from the group consisting of SEQ identifiers: C502-C508 in Table 3C. In some embodiments, the prime editing composition further comprises a ngRNA that comprises a spacer comprising a sequence selected from the group consisting of SEQ identifiers: D17, D35, D63-D70, and D93-D97 in Table 3D. In some embodiments, the PEgRNA comprises an editing template comprising a sequence selected from the group consisting of SEQ ID Nos: 4528-4534 in Table 3C. In some embodiments, the prime editing composition further comprises a ngRNA that comprises a spacer comprising a sequence selected from the group consisting of SEQ ID Nos: 19650, 19668, 19696-19703, and 19726-19730 in Table 3D.

In some embodiments, the PEgRNA comprises a spacer comprising the sequence of SEQ identifier: A42 in Table 3A. In some embodiments, the PEgRNA comprises a spacer comprising the sequence of SEQ ID No: 390 in Table 3A. In some embodiments, the PEgRNA comprises a PBS comprising a sequence selected from the group consisting of SEQ identifiers: B190-B198 in Table 3B. In some embodiments, the PEgRNA comprises a PBS comprising a sequence selected from the group consisting of SEQ ID Nos: 1360-1366 in Table 3B. In some embodiments, the PEgRNA comprises a PBS comprising a sequence selected from the group consisting of sequences: ATTATGTA and ATTATGTAT. In some embodiments, the PEgRNA comprises an editing template comprising a sequence selected from the group consisting of SEQ identifiers: C509-C513 in Table 3C. In some embodiments, the prime editing composition further comprises a ngRNA that comprises a spacer comprising a sequence selected from the group consisting of SEQ identifiers: D39-D54 in Table 3D. In some embodiments, the PEgRNA comprises an editing template comprising a sequence selected from the group consisting of SEQ ID Nos: 4535-4539 in Table 3C. In some embodiments, the prime editing composition further comprises a ngRNA that comprises a spacer comprising a sequence selected from the group consisting of SEQ ID Nos: 19672-19687 in Table 3D.

In some embodiments, the PEgRNA comprises a spacer comprising a sequence selected from the group consisting of SEQ identifiers: A27-A28 in Table 3A. In some embodiments, the PEgRNA comprises a spacer comprising a sequence selected from the group consisting of SEQ ID Nos: 375-376 in Table 3A. In some embodiments, the PEgRNA comprises a PBS comprising a sequence selected from the group consisting of SEQ identifiers: B118-B126 in Table 3B. In some embodiments, the PEgRNA comprises a PBS comprising a sequence selected from the group consisting of SEQ ID Nos: 1304-1310 in Table 3B. In some embodiments, the PEgRNA comprises a PBS comprising a sequence selected from the group consisting of sequences: GTACATTC and GTACATTCA. In some embodiments, the PEgRNA comprises an editing template comprising a sequence selected from the group consisting of identifiers: C514-C526 in Table 3C. In some embodiments, the prime editing composition further comprises a ngRNA that comprises a spacer comprising a sequence selected from the group consisting of SEQ identifiers: D39-D62 in Table 3D. In some embodiments, the PEgRNA comprises an editing template comprising a sequence selected from the group consisting of SEQ ID Nos: 4540-4552 in Table 3C. In some embodiments, the prime editing composition further comprises a ngRNA that comprises a spacer comprising a sequence selected from the group consisting of SEQ ID Nos: 19672-19695 in Table 3D.

In some embodiments, the PEgRNA comprises a spacer comprising a sequence selected from the group consisting of SEQ identifiers: A43-A44 in Table 3A. In some embodiments, the PEgRNA comprises a spacer comprising a sequence selected from the group consisting of SEQ ID Nos: 391-392 in Table 3A. In some embodiments, the PEgRNA comprises a PBS comprising a sequence selected from the group consisting of SEQ identifiers: B199-B207 in Table 3B. In some embodiments, the PEgRNA comprises a PBS comprising a sequence selected from the group consisting of SEQ ID Nos: 1367-1373 in Table 3B. In some embodiments, the PEgRNA comprises a PBS comprising a sequence selected from the group consisting of sequences: TATTTAGG and TATTTAGGG. In some embodiments, the PEgRNA comprises an editing template comprising a sequence selected from the group consisting of SEQ identifiers: C527-C557 in Table 3C. In some embodiments, the prime editing composition further comprises a ngRNA that comprises a spacer comprising a sequence selected from the group consisting of SEQ identifiers: D39-D61, D90, D124-D130 in Table 3D. In some embodiments, the PEgRNA comprises an editing template comprising a sequence selected from the group consisting of SEQ ID Nos: 4553-4583 in Table 3C. In some embodiments, the prime editing composition further comprises a ngRNA that comprises a spacer comprising a sequence selected from the group consisting of SEQ ID Nos: 19672-19694, 19723, 19757-19763 in Table 3D.

In some embodiments, the PEgRNA comprises a spacer comprising a sequence selected from the group consisting of SEQ identifiers: A30-A31 in Table 3A. In some embodiments, the PEgRNA comprises a spacer comprising a sequence selected from the group consisting of SEQ ID Nos: 378-379 in Table 3A. In some embodiments, the PEgRNA comprises a PBS comprising a sequence selected from the group consisting of SEQ identifiers: B136-B144 in Table 3B. In some embodiments, the PEgRNA comprises a PBS comprising a sequence selected from the group consisting of SEQ ID Nos: 1318-1324 in Table 3B. In some embodiments, the PEgRNA comprises a PBS comprising a sequence selected from the group consisting of sequences: GAAAAAAC and GAAAAAACA. In some embodiments, the PEgRNA comprises an editing template comprising a sequence selected from the group consisting of SEQ identifiers: C558-C568 in Table 3C. In some embodiments, the prime editing composition further comprises a ngRNA that comprises a spacer comprising a sequence selected from the group consisting of SEQ identifiers: D10-D37, and D92 in Table 3D. In some embodiments, the PEgRNA comprises an editing template comprising a sequence selected from the group consisting of SEQ ID Nos: 4584-4594 in Table 3C. In some embodiments, the prime editing composition further comprises a ngRNA that comprises a spacer comprising a sequence selected from the group consisting of SEQ ID Nos: 19643-19670, and 19725 in Table 3D.

In some embodiments, the PEgRNA comprises a spacer comprising a sequence selected from the group consisting of SEQ identifiers: 380, 381, 393, and 394 in Table 3A. In some embodiments, the PEgRNA comprises a spacer comprising a sequence selected from the group consisting of SEQ ID Nos: A32, A33, A45, and A46 in Table 3A. In some embodiments, the PEgRNA comprises a PBS comprising a sequence selected from the group consisting of SEQ identifiers: B145-B153 in Table 3B. In some embodiments, the PEgRNA comprises a PBS comprising a sequence selected from the group consisting of SEQ ID Nos: 1325-1331 in Table 3B. In some embodiments, the PEgRNA comprises a PBS comprising a sequence selected from the group consisting of sequences: AACATAAA and AACATAAAT. In some embodiments, the PEgRNA comprises an editing template comprising a sequence selected from the group consisting of SEQ identifiers: C569-C593 in Table 3C. In some embodiments, the prime editing composition further comprises a ngRNA that comprises a spacer comprising a sequence selected from the group consisting of SEQ identifiers: D11-D27, D33-D37, D65, D67, D70, D79-D83, D105, D122, and D131-D135 in Table 3D. In some embodiments, the PEgRNA comprises an editing template comprising a sequence selected from the group consisting of SEQ ID Nos: 4595-4619 in Table 3C. In some embodiments, the prime editing composition further comprises a ngRNA that comprises a spacer comprising a sequence selected from the group consisting of SEQ ID Nos: 19644-19660, 19666-19670, 19698, 19700, 19703, 19712-19716, 19738, 19755, and 19764-19768 in Table 3D.

In some embodiments, the PEgRNA comprises a spacer comprising a sequence selected from the group consisting of SEQ identifiers: A34-A35 in Table 3A. In some embodiments, the PEgRNA comprises a spacer comprising a sequence selected from the group consisting of SEQ ID Nos: 382-383 in Table 3A. In some embodiments, the PEgRNA comprises a PBS comprising a sequence selected from the group consisting of SEQ identifiers: B154-B162 in Table 3B. In some embodiments, the PEgRNA comprises a PBS comprising a sequence selected from the group consisting of SEQ ID Nos: 1332-1338 in Table 3B. In some embodiments, the PEgRNA comprises a PBS comprising a sequence selected from the group consisting of sequences: AAAAAACA and AAAAAACAT. In some embodiments, the PEgRNA comprises an editing template comprising a sequence selected from the group consisting of SEQ identifiers: C594-C613 in Table 3C. In some embodiments, the prime editing composition further comprises a ngRNA that comprises a spacer comprising a sequence selected from the group consisting of SEQ identifiers: D67, D95, D102-D119 in Table 3D. In some embodiments, the PEgRNA comprises an editing template comprising a sequence selected from the group consisting of SEQ ID Nos: 4620-4639 in Table 3C. In some embodiments, the prime editing composition further comprises a ngRNA that comprises a spacer comprising a sequence selected from the group consisting of SEQ ID Nos: 19700, 19728, 19735-19752 in Table 3D.

In some embodiments, the PEgRNA comprises a spacer comprising a sequence selected from the group consisting of SEQ identifiers: A47-A49 in Table 3A. In some embodiments, the PEgRNA comprises a spacer comprising a sequence selected from the group consisting of SEQ ID Nos: 395-397 in Table 3A. In some embodiments, the PEgRNA comprises a PBS comprising a sequence selected from the group consisting of SEQ identifiers: B208-B216 in Table 3B. In some embodiments, the PEgRNA comprises a PBS comprising a sequence selected from the group consisting of SEQ ID Nos: 1374-1380 in Table 3B. In some embodiments, the PEgRNA comprises a PBS comprising a sequence selected from the group consisting of sequences: ACATAAAT and ACATAAATC. In some embodiments, the PEgRNA comprises an editing template comprising a sequence selected from the group consisting of SEQ identifiers: C614-C637 in Table 3C. In some embodiments, the prime editing composition further comprises a ngRNA that comprises a spacer comprising a sequence selected from the group consisting of SEQ identifiers: D17, D35, D63-D70 and D93-D97 in Table 3D. In some embodiments, the PEgRNA comprises an editing template comprising a sequence selected from the group consisting of SEQ ID Nos: 4640-4663 in Table 3C. In some embodiments, the prime editing composition further comprises a ngRNA that comprises a spacer comprising a sequence selected from the group consisting of SEQ ID Nos: 19650, 19668, 19696-19703 and 19726-19730 in Table 3D.

In some embodiments, the editing target sequence is in exon 4 of the CFTR gene.

In some embodiments, the editing target sequence or the complementary sequence on the CFTR gene thereof comprises a mutation that encodes an amino acid substitution R117P, R117H, R117L, R117G, E116Q, K114*, E115*, E116*, and/or A120P, relative to a wild type CFTR polypeptide set forth in SEQ ID NO: 19, wherein * refers to a non-sense mutation, and wherein the editing template encodes an intended nucleotide edit that corrects the mutation. In some embodiments, the editing target sequence or the complementary sequence on the CFTR gene thereof comprises a G→→C mutation (c.350G>C) at position 117,530,975, a G→→C mutation (c.350G>A) at position 117,530,975, a G→→C mutation (c.350G>T) at position 117,530,975 a A→→T mutation (c.340A>T) at position 117,530,965, a deletion of AAG (c.340_342delAAG) at position 117,530,965, a deletion of 13 nucleotides AAGGAGGAACGCT (SEQ ID NO: 347)(c.341_353del13) at position 117,530,966, a G→→T mutation (c.343G>T) at position 117,530,968, a deletion of GGA (c.345_347delGGA) at position 117,530,970, a G→→C mutation (c.346G>C) at position 117,530,971, a G→→T mutation (c.346G>T) at position 117,530,971, a C→→G mutation (c.349C>G) at position 117,530,974, a deletion of C (c.357delC) at position 117,530,982, and/or a G→→C mutation (c.358G>C) at position 117,530,983 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide. In some embodiments, the editing template comprises a wild type CFTR gene sequence. Accordingly, in some embodiments, the newly synthesized single stranded DNA encoded by the editing template is capable of correcting the c.350G>C mutation, the c.350G>A mutation, the c.350G>T mutation, the c.340A>T mutation, the c.340_342delAAG mutation, the c.341_353del13 mutation, the c.343G>T mutation, the c.345_347delGGA mutation, the c.346G>C mutation, the c.346G>T mutation, the c.349C>G mutation, the c.357delC mutation, and/or the c.358G>C mutation in the target CFTR gene. In some embodiments, the newly synthesized single stranded DNA encoded by the editing template is capable of correcting all of the c.350G>C mutation, the c.350G>A mutation, the c.350G>T mutation, the c.340A>T mutation, the c.340_342delAAG mutation, the c.341_353del13 mutation, the c.343G>T mutation, the c.345_347delGGA mutation, the c.346G>C mutation, the c.346G>T mutation, the c.349C>G mutation, the c.357delC mutation, and the c.358G>C mutation in the target CFTR gene. In some embodiments, two or more of the c.350G>C mutation, the c.350G>A mutation, the c.350G>T mutation, the c.340A>T mutation, the c.340_342delAAG mutation, the c.341_353del13 mutation, the c.343G>T mutation, the c.345_347delGGA mutation, the c.346G>C mutation, the c.346G>T mutation, the c.349C>G mutation, the c.357delC mutation, and the c.358G>C mutation are located on the same CFTR gene. In some embodiments, the newly synthesized single stranded DNA encoded by the editing template is capable of correcting the c.350G>C mutation, the c.350G>A mutation, the c.350G>T mutation, the c.340A>T mutation, the c.340_342delAAG mutation, the c.341_353del13 mutation, the c.343G>T mutation, the c.345_347delGGA mutation, the c.346G>C mutation, the c.346G>T mutation, the c.349C>G mutation, the c.357delC mutation, and the c.358G>C mutation that are located on different CFTR genes. For example, in some embodiments, a subject may comprise two copies of CFTR genes, each comprising a different mutation selected from the group consisting of the c.350G>C mutation, the c.350G>A mutation, the c.350G>T mutation, the c.340A>T mutation, the c.340_342delAAG mutation, the c.341_353del13 mutation, the c.343G>T mutation, the c.345_347delGGA mutation, the c.346G>C mutation, the c.346G>T mutation, the c.349C>G mutation, the c.357delC mutation, and the c.358G>C mutation, all of which may be corrected by an editing template of a single PEgRNA. In some embodiments, the editing template is capable of correcting one or more, or all of the c.350G>C mutation, the c.350G>A mutation, the c.350G>T mutation, the c.340A>T mutation, the c.340_342delAAG mutation, the c.341_353del13 mutation, the c.343G>T mutation, the c.345_347delGGA mutation, the c.346G>C mutation, the c.346G>T mutation, the c.349C>G mutation, the c.357delC mutation, and the c.358G>C mutation that are in different subjects, wherein each subject may have one or more of the mutations.

In some embodiments, the PEgRNA comprises a spacer comprising the sequence of SEQ identifier: A50 in Table 3A. In some embodiments, the PEgRNA comprises a spacer comprising the sequence of SEQ ID No: 398 in Table 3A. In some embodiments, the PEgRNA comprises a PBS comprising a sequence selected from the group consisting of SEQ identifiers: B217-B225 in Table 3B. In some embodiments, the PEgRNA comprises a PBS comprising a sequence selected from the group consisting of SEQ ID Nos: 1381-1387 in Table 3B. In some embodiments, the PEgRNA comprises a PBS comprising a sequence selected from the group consisting of sequences: GCTTATGC and GCTTATGCC. In some embodiments, the PEgRNA comprises an editing template comprising a sequence selected from the group consisting of SEQ identifiers: C638-C650 in Table 3C. In some embodiments, the prime editing composition further comprises a ngRNA that comprises a spacer comprising a sequence selected from the group consisting of SEQ identifiers: D136-D157 in Table 3D. In some embodiments, the PEgRNA comprises an editing template comprising a sequence selected from the group consisting of SEQ ID Nos: 4394-4676 in Table 3C. In some embodiments, the prime editing composition further comprises a ngRNA that comprises a spacer comprising a sequence selected from the group consisting of SEQ ID Nos: 19769-19790 in Table 3D.

In some embodiments, the PEgRNA comprises a spacer comprising the sequence of SEQ identifiers: A51 in Table 3A. In some embodiments, the PEgRNA comprises a spacer comprising the sequence of SEQ ID No: 399 in Table 3A. In some embodiments, the PEgRNA comprises a PBS comprising a sequence selected from the group consisting of SEQ identifiers: B226-B234 in Table 3B. In some embodiments, the PEgRNA comprises a PBS comprising a sequence selected from the group consisting of SEQ ID Nos: 1388-1394 in Table 3B. In some embodiments, the PEgRNA comprises a PBS comprising a sequence selected from the group consisting of sequences: TATCTAGG and TATCTAGGC. In some embodiments, the PEgRNA comprises an editing template comprising a sequence selected from the group consisting of SEQ identifiers: C651-C676 in Table 3C. In some embodiments, the prime editing composition further comprises a ngRNA that comprises a spacer comprising a sequence selected from the group consisting of SEQ identifiers: D136-D155, and D158-D161 in Table 3D. In some embodiments, the PEgRNA comprises an editing template comprising a sequence selected from the group consisting of SEQ ID Nos: 4677-4702 in Table 3C. In some embodiments, the prime editing composition further comprises a ngRNA that comprises a spacer comprising a sequence selected from the group consisting of SEQ ID Nos: 19769-19788, and 19791-19794 in Table 3D.

In some embodiments, the PEgRNA comprises a spacer comprising a sequence selected from the group consisting of SEQ identifiers: A52-A53 in Table 3A. In some embodiments, the PEgRNA comprises a spacer comprising a sequence selected from the group consisting of SEQ ID Nos: 400-401 in Table 3A. In some embodiments, the PEgRNA comprises a PBS comprising a sequence selected from the group consisting of SEQ identifiers: B235-B243 in Table 3B. In some embodiments, the PEgRNA comprises a PBS comprising a sequence selected from the group consisting of SEQ ID Nos: 1395-1401 in Table 3B. In some embodiments, the PEgRNA comprises a PBS comprising a sequence selected from the group consisting of sequences: CATAGGCT and CATAGGCTT. In some embodiments, the PEgRNA comprises an editing template comprising a sequence selected from the group consisting of SEQ identifiers: C677-C694 in Table 3C. In some embodiments, the prime editing composition further comprises a ngRNA that comprises a spacer comprising a sequence selected from the group consisting of SEQ identifiers: D136-D157, and D161-D169 in Table 3D. In some embodiments, the PEgRNA comprises an editing template comprising a sequence selected from the group consisting of SEQ ID Nos: 4703-4720 in Table 3C. In some embodiments, the prime editing composition further comprises a ngRNA that comprises a spacer comprising a sequence selected from the group consisting of SEQ ID Nos: 19769-19790, and 19794-19802 in Table 3D.

In some embodiments, the PEgRNA comprises a spacer comprising a sequence selected from the group consisting of SEQ identifiers: A54-A55 in Table 3A. In some embodiments, the PEgRNA comprises a spacer comprising a sequence selected from the group consisting of SEQ ID Nos: 402-403 in Table 3A. In some embodiments, the PEgRNA comprises a PBS comprising a sequence selected from the group consisting of SEQ identifiers: B244-B252 in Table 3B. In some embodiments, the PEgRNA comprises a PBS comprising a sequence selected from the group consisting of SEQ ID Nos: 1402-1408 in Table 3B. In some embodiments, the PEgRNA comprises a PBS comprising a sequence selected from the group consisting of sequences: CATAGGAA and CATAGGAAG. In some embodiments, the PEgRNA comprises an editing template comprising a sequence selected from the group consisting of SEQ identifiers: C695-C730 in Table 3C. In some embodiments, the prime editing composition further comprises a ngRNA that comprises a spacer comprising a sequence selected from the group consisting of SEQ identifiers: D170-D195 in Table 3D. In some embodiments, the PEgRNA comprises an editing template comprising a sequence selected from the group consisting of SEQ ID Nos: 4721-4756 in Table 3C. In some embodiments, the prime editing composition further comprises a ngRNA that comprises a spacer comprising a sequence selected from the group consisting of SEQ ID Nos: 19803-19828 in Table 3D.

In some embodiments, the PEgRNA comprises a spacer comprising a sequence selected from the group consisting of SEQ identifiers: A56-A57 in Table 3A. In some embodiments, the PEgRNA comprises a spacer comprising a sequence selected from the group consisting of SEQ ID Nos: 404-405 in Table 3A. In some embodiments, the PEgRNA comprises a PBS comprising a sequence selected from the group consisting of SEQ identifiers: B253-B261 in Table 3B. In some embodiments, the PEgRNA comprises a PBS comprising a sequence selected from the group consisting of SEQ ID Nos: 1409-1415 in Table 3B. In some embodiments, the PEgRNA comprises a PBS comprising a sequence selected from the group consisting of sequences: TTGTTATC and TTGTTATCC. In some embodiments, the PEgRNA comprises an editing template comprising a sequence selected from the group consisting of SEQ identifiers: C731-C761 in Table 3C. In some embodiments, the prime editing composition further comprises a ngRNA that comprises a spacer comprising a sequence selected from the group consisting of SEQ identifiers: D172-D190, and D196-D207 in Table 3D. In some embodiments, the PEgRNA comprises an editing template comprising a sequence selected from the group consisting of SEQ ID Nos: 4757-4787 in Table 3C. In some embodiments, the prime editing composition further comprises a ngRNA that comprises a spacer comprising a sequence selected from the group consisting of SEQ ID Nos: 19805-19823, and 19829-19840 in Table 3D.

In some embodiments, the PEgRNA comprises a spacer comprising a sequence selected from the group consisting of SEQ identifiers: A58-A59 in Table 3A. In some embodiments, the PEgRNA comprises a spacer comprising a sequence selected from the group consisting of SEQ ID Nos: 406-407 in Table 3A. In some embodiments, the PEgRNA comprises a PBS comprising a sequence selected from the group consisting of SEQ identifiers: B262-B270 in Table 3B. In some embodiments, the PEgRNA comprises a PBS comprising a sequence selected from the group consisting of SEQ ID Nos: 1416-1422 in Table 3B. In some embodiments, the PEgRNA comprises a PBS comprising a sequence selected from the group consisting of sequences: CGATTTAT and CGATTTATC. In some embodiments, the PEgRNA comprises an editing template comprising a sequence selected from the group consisting of SEQ identifiers: C762-C823 in Table 3C. In some embodiments, the prime editing composition further comprises a ngRNA that comprises a spacer comprising a sequence selected from the group consisting of SEQ identifiers: D208-D226 in Table 3D. In some embodiments, the PEgRNA comprises an editing template comprising a sequence selected from the group consisting of SEQ ID Nos: 4788-4849 in Table 3C. In some embodiments, the prime editing composition further comprises a ngRNA that comprises a spacer comprising a sequence selected from the group consisting of SEQ ID Nos: 19841-19859 in Table 3D.

In some embodiments, the PEgRNA comprises a spacer comprising a sequence selected from the group consisting of SEQ identifiers: A60-A61 in Table 3A. In some embodiments, the PEgRNA comprises a spacer comprising a sequence selected from the group consisting of SEQ ID Nos: 408-409 in Table 3A. In some embodiments, the PEgRNA comprises a PBS comprising a sequence selected from the group consisting of SEQ identifiers: B271-B279 in Table 3B. In some embodiments, the PEgRNA comprises a PBS comprising a sequence selected from the group consisting of SEQ ID Nos: 1423-1429 in Table 3B. In some embodiments, the PEgRNA comprises a PBS comprising a sequence selected from the group consisting of sequences: CCTTCTCT and CCTTCTCTT. In some embodiments, the PEgRNA comprises an editing template comprising a sequence selected from the group consisting of SEQ identifiers: C824-C835 in Table 3C. In some embodiments, the prime editing composition further comprises a ngRNA that comprises a spacer comprising a sequence selected from the group consisting of SEQ identifiers: D210-D215, D219-D224, and D227-D230 in Table 3D. In some embodiments, the PEgRNA comprises an editing template comprising a sequence selected from the group consisting of SEQ ID Nos: 4850-4861 in Table 3C. In some embodiments, the prime editing composition further comprises a ngRNA that comprises a spacer comprising a sequence selected from the group consisting of SEQ ID Nos: 19843-19848, 19852-19857, and 19860-19863 in Table 3D.

In some embodiments, the PEgRNA comprises a spacer comprising the sequence of SEQ identifier A62 in Table 3A. In some embodiments, the PEgRNA comprises a spacer comprising the sequence of SEQ ID No 410 in Table 3A. In some embodiments, the PEgRNA comprises a PBS comprising a sequence selected from the group consisting of SEQ identifiers: B280-B288 in Table 3B. In some embodiments, the PEgRNA comprises a PBS comprising a sequence selected from the group consisting of SEQ ID Nos: 1430-1436 in Table 3B. In some embodiments, the PEgRNA comprises a PBS comprising a sequence selected from the group consisting of sequences: GCCTTCTC and GCCTTCTCT. In some embodiments, the PEgRNA comprises an editing template comprising a sequence selected from the group consisting of SEQ identifiers: C836-C842 in Table 3C. In some embodiments, the prime editing composition further comprises a ngRNA that comprises a spacer comprising a sequence selected from the group consisting of SEQ identifiers: D137-D157 in Table 3D. In some embodiments, the PEgRNA comprises an editing template comprising a sequence selected from the group consisting of SEQ ID Nos: 4862-4868 in Table 3C. In some embodiments, the prime editing composition further comprises a ngRNA that comprises a spacer comprising a sequence selected from the group consisting of SEQ ID Nos: 19770-19790 in Table 3D.

In some embodiments, the PEgRNA comprises a spacer comprising a sequence selected from the group consisting of SEQ ID identifiers: A63-A66 in Table 3A. In some embodiments, the PEgRNA comprises a spacer comprising a sequence selected from the group consisting of SEQ ID Nos: 411-414 in Table 3A. In some embodiments, the PEgRNA comprises a PBS comprising a sequence selected from the group consisting of SEQ identifiers: B289-B297 in Table 3B. In some embodiments, the PEgRNA comprises a PBS comprising a sequence selected from the group consisting of SEQ ID Nos: 1437-1443 in Table 3B. In some embodiments, the PEgRNA comprises a PBS comprising a sequence selected from the group consisting of sequences: TCGCGATT and TCGCGATTT. In some embodiments, the PEgRNA comprises an editing template comprising a sequence selected from the group consisting of SEQ identifiers: C843-C873 in Table 3C. In some embodiments, the prime editing composition further comprises a ngRNA that comprises a spacer comprising a sequence selected from the group consisting of SEQ identifiers: D136-D154, D158-D161, D219, D225, D226, and D231-D247 in Table 3D. In some embodiments, the PEgRNA comprises an editing template comprising a sequence selected from the group consisting of SEQ ID Nos: 4869-4899 in Table 3C. In some embodiments, the prime editing composition further comprises a ngRNA that comprises a spacer comprising a sequence selected from the group consisting of SEQ ID Nos: 19769-19787, 19791-19794, 19852, 19858, 19859, and 19864-19880 in Table 3D.

In some embodiments, the PEgRNA comprises a spacer comprising a sequence selected from the group consisting of SEQ ID identifiers: A67-A68 in Table 3A. In some embodiments, the PEgRNA comprises a spacer comprising a sequence selected from the group consisting of SEQ ID Nos: 415-416 in Table 3A. In some embodiments, the PEgRNA comprises a PBS comprising a sequence selected from the group consisting of SEQ identifiers: B298-B306 in Table 3B. In some embodiments, the PEgRNA comprises a PBS comprising a sequence selected from the group consisting of SEQ ID Nos: 1444-1450 in Table 3B. In some embodiments, the PEgRNA comprises a PBS comprising a sequence selected from the group consisting of sequences: TTATCCGG and TTATCCGGG. In some embodiments, the PEgRNA comprises an editing template comprising a sequence selected from the group consisting of SEQ identifiers: C874-C901 in Table 3C. In some embodiments, the prime editing composition further comprises a ngRNA that comprises a spacer comprising a sequence selected from the group consisting of SEQ identifiers: D170-D190, D196-D207, and 248-D249 of Table 3D. In some embodiments, the PEgRNA comprises an editing template comprising a sequence selected from the group consisting of SEQ ID Nos: 4900-4927 in Table 3C. In some embodiments, the prime editing composition further comprises a ngRNA that comprises a spacer comprising a sequence selected from the group consisting of SEQ ID Nos: 19803-19823, 19829-19840, and 19881-19882 of Table 3D.

In some embodiments, the PEgRNA comprises a spacer comprising a sequence selected from the group consisting of SEQ ID identifiers: A69-A70 of Table 3A. In some embodiments, the PEgRNA comprises a spacer comprising a sequence selected from the group consisting of SEQ ID Nos: 417-418 of Table 3A. In some embodiments, the PEgRNA comprises a PBS comprising a sequence selected from the group consisting of SEQ identifiers: B307-B315 of Table 3B. In some embodiments, the PEgRNA comprises a PBS comprising a sequence selected from the group consisting of SEQ ID Nos: 1451-1457 of Table 3B. In some embodiments, the PEgRNA comprises a PBS comprising a sequence selected from the group consisting of sequences: TTTATCTA and TTTATCTAG. In some embodiments, the PEgRNA comprises an editing template comprising a sequence selected from the group consisting of SEQ identifiers: C902-C929 of Table 3C. In some embodiments, the prime editing composition further comprises a ngRNA that comprises a spacer comprising a sequence selected from the group consisting of SEQ identifiers: D136-D155, D158-D169 of Table 3D. In some embodiments, the PEgRNA comprises an editing template comprising a sequence selected from the group consisting of SEQ ID Nos: 4928-4955 of Table 3C. In some embodiments, the prime editing composition further comprises a ngRNA that comprises a spacer comprising a sequence selected from the group consisting of SEQ ID Nos: 19769-19788, 19791-19802 of Table 3D.

In some embodiments, the PEgRNA comprises a spacer comprising a sequence selected from the group consisting of SEQ ID identifiers: A71-A72 of Table 3A. In some embodiments, the PEgRNA comprises a spacer comprising a sequence selected from the group consisting of SEQ ID Nos: 419-420 of Table 3A. In some embodiments, the PEgRNA comprises a PBS comprising a sequence selected from the group consisting of SEQ identifiers: B316-B324 of Table 3B. In some embodiments, the PEgRNA comprises a PBS comprising a sequence selected from the group consisting of SEQ ID Nos: 1458-1464 of Table 3B. In some embodiments, the PEgRNA comprises a PBS comprising a sequence selected from the group consisting of sequences: ATCGCGAT and ATCGCGATT. In some embodiments, the PEgRNA comprises an editing template comprising a sequence selected from the group consisting of SEQ identifiers: C930-C960 of Table 3C. In some embodiments, the prime editing composition further comprises a ngRNA that comprises a spacer comprising a sequence selected from the group consisting of SEQ identifiers: D136-D154, D158-D161, D236-D240, and D250 of Table 3D. In some embodiments, the PEgRNA comprises an editing template comprising a sequence selected from the group consisting of SEQ ID Nos: 4956-4986 of Table 3C. In some embodiments, the prime editing composition further comprises a ngRNA that comprises a spacer comprising a sequence selected from the group consisting of SEQ ID Nos: 19769-19787, 19791-19794, 19869-19873, and 19883 of Table 3D.

In some embodiments, the PEgRNA comprises a spacer comprising the sequence of SEQ identifier: A73 of Table 3A. In some embodiments, the PEgRNA comprises a spacer comprising the sequence of SEQ ID No: 421 of Table 3A. In some embodiments, the PEgRNA comprises a PBS comprising a sequence selected from the group consisting of SEQ identifiers: B325-B333 of Table 3B. In some embodiments, the PEgRNA comprises a PBS comprising a sequence selected from the group consisting of SEQ ID Nos: 1465-1471 of Table 3B. In some embodiments, the PEgRNA comprises a PBS comprising a sequence selected from the group consisting of sequences: TATCGCGA and TATCGCGAT. In some embodiments, the PEgRNA comprises an editing template comprising a sequence selected from the group consisting of SEQ identifiers: C961-C991 of Table 3C. In some embodiments, the prime editing composition further comprises a ngRNA that comprises a spacer comprising a sequence selected from the group consisting of SEQ identifiers: D136-D154, D158-D161, and D250 of Table 3D. In some embodiments, the PEgRNA comprises an editing template comprising a sequence selected from the group consisting of SEQ ID Nos: 4987-5017 of Table 3C. In some embodiments, the prime editing composition further comprises a ngRNA that comprises a spacer comprising a sequence selected from the group consisting of SEQ ID Nos: 19769-19787, 19791-19794, and 19883 of Table 3D.

In some embodiments, the PEgRNA comprises a spacer comprising a sequence selected from the group consisting of SEQ identifiers: A74-A77 of Table 3A. In some embodiments, the PEgRNA comprises a spacer comprising a sequence selected from the group consisting of SEQ ID Nos: 422-425 of Table 3A. In some embodiments, the PEgRNA comprises a PBS comprising a sequence selected from the group consisting of SEQ identifiers: B334-B342 of Table 3B. In some embodiments, the PEgRNA comprises a PBS comprising a sequence selected from the group consisting of SEQ ID Nos: 1472-1478 of Table 3B. In some embodiments, the PEgRNA comprises a PBS comprising a sequence selected from the group consisting of sequences: GTTATCCG and GTTATCCGG. In some embodiments, the PEgRNA comprises an editing template comprising a sequence selected from the group consisting of SEQ identifiers: C992-C1049 of Table 3C. In some embodiments, the prime editing composition further comprises a ngRNA that comprises a spacer comprising a sequence selected from the group consisting of SEQ identifiers: D176, D177, D207, and D251-D290 of Table 3D. In some embodiments, the PEgRNA comprises an editing template comprising a sequence selected from the group consisting of SEQ ID Nos: 5018-5075 of Table 3C. In some embodiments, the prime editing composition further comprises a ngRNA that comprises a spacer comprising a sequence selected from the group consisting of SEQ ID Nos: 19809, 19810, 19840, and 19884-19923 of Table 3D.

In some embodiments, the PEgRNA comprises a spacer comprising a sequence selected from the group consisting of SEQ identifiers: A78-A79 of Table 3AB. In some embodiments, the PEgRNA comprises a spacer comprising a sequence selected from the group consisting of SEQ ID Nos: 426-427 of Table 3AB. In some embodiments, the PEgRNA comprises a PBS comprising a sequence selected from the group consisting of SEQ identifiers: B343-B351 of Table 3B. In some embodiments, the PEgRNA comprises a PBS comprising a sequence selected from the group consisting of SEQ ID Nos: 1479-1485 of Table 3B. In some embodiments, the PEgRNA comprises a PBS comprising a sequence selected from the group consisting of sequences: TTCTTCCC and TTCTTCCCA. In some embodiments, the PEgRNA comprises an editing template comprising a sequence selected from the group consisting of SEQ identifiers: C1050-C1055 of Table 3C. In some embodiments, the prime editing composition further comprises a ngRNA that comprises a spacer comprising a sequence selected from the group consisting of SEQ identifiers: D251-D 271, and D291-D298 of Table 3D. In some embodiments, the PEgRNA comprises an editing template comprising a sequence selected from the group consisting of SEQ ID Nos: 5076-5081 of Table 3C. In some embodiments, the prime editing composition further comprises a ngRNA that comprises a spacer comprising a sequence selected from the group consisting of SEQ ID Nos: 19884-19904, and 19924-19931 of Table 3D.

In some embodiments, the PEgRNA comprises a spacer comprising the sequence of SEQ identifier: A80 of Table 3A. In some embodiments, the PEgRNA comprises a spacer comprising the sequence of SEQ ID No: 428 of Table 3A. In some embodiments, the PEgRNA comprises a PBS comprising a sequence selected from the group consisting of SEQ identifiers: B352-B360 of Table 3B. In some embodiments, the PEgRNA comprises a PBS comprising a sequence selected from the group consisting of SEQ ID Nos: 1486-1492 of Table 3B. In some embodiments, the PEgRNA comprises a PBS comprising a sequence selected from the group consisting of sequences: ATTCTTCC and ATTCTTCCC. In some embodiments, the PEgRNA comprises an editing template comprising a sequence selected from the group consisting of SEQ identifiers: C1056-C1059 of Table 3C. In some embodiments, the prime editing composition further comprises a ngRNA that comprises a spacer comprising a sequence selected from the group consisting of SEQ identifiers: D170-D187 of Table 3D. In some embodiments, the PEgRNA comprises an editing template comprising a sequence selected from the group consisting of SEQ ID Nos: 5082-5085 of Table 3C. In some embodiments, the prime editing composition further comprises a ngRNA that comprises a spacer comprising a sequence selected from the group consisting of SEQ ID Nos: 19803-19820 of Table 3D.

In some embodiments, the PEgRNA comprises a spacer comprising a sequence selected from the group consisting of SEQ identifiers: A81-A82 of Table 3A. In some embodiments, the PEgRNA comprises a spacer comprising a sequence selected from the group consisting of SEQ ID Nos: 429-430 of Table 3A. In some embodiments, the PEgRNA comprises a PBS comprising a sequence selected from the group consisting of SEQ identifiers: B361-B369 of Table 3B. In some embodiments, the PEgRNA comprises a PBS comprising a sequence selected from the group consisting of SEQ ID Nos: 1493-1499 of Table 3B. In some embodiments, the PEgRNA comprises a PBS comprising a sequence selected from the group consisting of sequences: GAAGCTAT and GAAGCTATG. In some embodiments, the PEgRNA comprises an editing template comprising a sequence selected from the group consisting of SEQ identifiers: C1060-C1072 of Table 3C. In some embodiments, the prime editing composition further comprises a ngRNA that comprises a spacer comprising a sequence selected from the group consisting of SEQ identifiers: D170-D188, D196-D202, D248, and D249 of Table 3D. In some embodiments, the PEgRNA comprises an editing template comprising a sequence selected from the group consisting of SEQ ID Nos: 5086-5098 of Table 3C. In some embodiments, the prime editing composition further comprises a ngRNA that comprises a spacer comprising a sequence selected from the group consisting of SEQ ID Nos: 19803-19821, 19829-19835, 19881, and 19882 of Table 3D.

In some embodiments, the PEgRNA comprises a spacer comprising a sequence selected from the group consisting of SEQ identifiers: A83-A84 of Table 3A. In some embodiments, the PEgRNA comprises a spacer comprising a sequence selected from the group consisting of SEQ ID Nos: 431-432 of Table 3A. In some embodiments, the PEgRNA comprises a PBS comprising a sequence selected from the group consisting of SEQ identifiers: B370-B378 of Table 3B. In some embodiments, the PEgRNA comprises a PBS comprising a sequence selected from the group consisting of SEQ ID Nos: 1500-1506 of Table 3B. In some embodiments, the PEgRNA comprises a PBS comprising a sequence selected from the group consisting of sequences: ATAGGCTT and ATAGGCTTA. In some embodiments, the PEgRNA comprises an editing template comprising a sequence selected from the group consisting of SEQ identifiers: C1073-C1106 of Table 3C. In some embodiments, the prime editing composition further comprises a ngRNA that comprises a spacer comprising a sequence selected from the group consisting of SEQ identifiers: D210-D215, D219, D224, and D227-D230 of Table 3D. In some embodiments, the PEgRNA comprises an editing template comprising a sequence selected from the group consisting of SEQ ID Nos: 5099-5132 of Table 3C. In some embodiments, the prime editing composition further comprises a ngRNA that comprises a spacer comprising a sequence selected from the group consisting of SEQ ID Nos: 19843-19848, 19852, 19857, and 19860-19863 of Table 3D.

In some embodiments, the PEgRNA comprises a spacer comprising a sequence selected from the group consisting of SEQ identifiers: A85-A87 of Table 3A. In some embodiments, the PEgRNA comprises a spacer comprising a sequence selected from the group consisting of SEQ ID Nos: 433-435 of Table 3A. In some embodiments, the PEgRNA comprises a PBS comprising a sequence selected from the group consisting of SEQ identifiers: B379-B387 of Table 3B. In some embodiments, the PEgRNA comprises a PBS comprising a sequence selected from the group consisting of SEQ ID Nos: 1507-1513 of Table 3B. In some embodiments, the PEgRNA comprises a PBS comprising a sequence selected from the group consisting of sequences: ATAGGAAG and ATAGGAAGC. In some embodiments, the PEgRNA comprises an editing template comprising a sequence selected from the group consisting of SEQ identifiers: C1107-C1123 of Table 3C. In some embodiments, the prime editing composition further comprises a ngRNA that comprises a spacer comprising a sequence selected from the group consisting of SEQ identifiers: D176, D177, D251, D254, D255, D262, D275-D279, D283-D287, D299, and D300 of Table 3D. In some embodiments, the PEgRNA comprises an editing template comprising a sequence selected from the group consisting of SEQ ID Nos: 5133-5149 of Table 3C. In some embodiments, the prime editing composition further comprises a ngRNA that comprises a spacer comprising a sequence selected from the group consisting of SEQ ID Nos: 19809, 19810, 19884, 19887, 19888, 19895, 19908-19912, 19916-19920, 19932, and 19933 of Table 3D.

In some embodiments, the PEgRNA comprises a spacer comprising a sequence selected from the group consisting of SEQ identifiers: A88-A91 of Table 3A. In some embodiments, the PEgRNA comprises a spacer comprising a sequence selected from the group consisting of SEQ ID Nos: 436-439 of Table 3A. In some embodiments, the PEgRNA comprises a PBS comprising a sequence selected from the group consisting of SEQ identifiers: B388-B396 of Table 3B. In some embodiments, the PEgRNA comprises a PBS comprising a sequence selected from the group consisting of SEQ ID Nos: 1514-1520 of Table 3B. In some embodiments, the PEgRNA comprises a PBS comprising a sequence selected from the group consisting of sequences: CGCGATTT and CGCGATTTA. In some embodiments, the PEgRNA comprises an editing template comprising a sequence selected from the group consisting of SEQ identifiers: C1124-C1185 of Table 3C. In some embodiments, the prime editing composition further comprises a ngRNA that comprises a spacer comprising a sequence selected from the group consisting of SEQ identifiers: D145, D209-D219, D220-D226, D231-D235, D241-D247, and D301-D306 of Table 3D. In some embodiments, the PEgRNA comprises an editing template comprising a sequence selected from the group consisting of SEQ ID Nos: 5150-5211 of Table 3C. In some embodiments, the prime editing composition further comprises a ngRNA that comprises a spacer comprising a sequence selected from the group consisting of SEQ ID Nos: 19778, 19842-19852, 19853-19859, 19864-19868, 19874-19880, and 19934-19939 of Table 3D.

In some embodiments, the PEgRNA comprises a spacer comprising a sequence selected from the group consisting of SEQ identifiers: A92-A93 of Table 3A. In some embodiments, the PEgRNA comprises a spacer comprising a sequence selected from the group consisting of SEQ ID Nos: 440-441 of Table 3A. In some embodiments, the PEgRNA comprises a PBS comprising a sequence selected from the group consisting of SEQ identifiers: B397-B405 of Table 3B. In some embodiments, the PEgRNA comprises a PBS comprising a sequence selected from the group consisting of SEQ ID Nos: 1521-1527 of Table 3B. In some embodiments, the PEgRNA comprises a PBS comprising a sequence selected from the group consisting of sequences: TGTTATCC and TGTTATCCG. In some embodiments, the PEgRNA comprises an editing template comprising a sequence selected from the group consisting of SEQ identifiers: C1186-C1245 of Table 3C. In some embodiments, the prime editing composition further comprises a ngRNA that comprises a spacer comprising a sequence selected from the group consisting of SEQ identifiers: D171-D195, D204-D207, and D307-D315 of Table 3D. In some embodiments, the PEgRNA comprises an editing template comprising a sequence selected from the group consisting of SEQ ID Nos: 5212-5271 of Table 3C. In some embodiments, the prime editing composition further comprises a ngRNA that comprises a spacer comprising a sequence selected from the group consisting of SEQ ID Nos: 19804-19828, 19837-19840, and 19940-19948 of Table 3D.

In some embodiments, the PEgRNA comprises a spacer comprising a sequence selected from the group consisting of SEQ identifiers: A94-A95 of Table 3A. In some embodiments, the PEgRNA comprises a spacer comprising a sequence selected from the group consisting of SEQ ID Nos: 442-443 of Table 3A. In some embodiments, the PEgRNA comprises a PBS comprising a sequence selected from the group consisting of SEQ identifiers: B406-B414 of Table 3B. In some embodiments, the PEgRNA comprises a PBS comprising a sequence selected from the group consisting of SEQ ID Nos: 1528-1534 of Table 3B. In some embodiments, the PEgRNA comprises a PBS comprising a sequence selected from the group consisting of sequences: TCATAGGA and TCATAGGAA. In some embodiments, the PEgRNA comprises an editing template comprising a sequence selected from the group consisting of identifiers: C1246-C1264 of Table 3C. In some embodiments, the prime editing composition further comprises a ngRNA that comprises a spacer comprising a sequence selected from the group consisting of SEQ identifiers: D170-D190, D196-D204, D248, and D249 of Table 3D. In some embodiments, the PEgRNA comprises an editing template comprising a sequence selected from the group consisting of SEQ ID Nos: 5272-5290 of Table 3C. In some embodiments, the prime editing composition further comprises a ngRNA that comprises a spacer comprising a sequence selected from the group consisting of SEQ ID Nos: 19803-19823, 19829-19837, 19881, and 19882 of Table 3D.

In some embodiments, the PEgRNA comprises a spacer comprising a sequence selected from the group consisting of SEQ identifiers: A96-A97 of Table 3A. In some embodiments, the PEgRNA comprises a spacer comprising a sequence selected from the group consisting of SEQ ID Nos: 444-445 of Table 3A. In some embodiments, the PEgRNA comprises a PBS comprising a sequence selected from the group consisting of SEQ identifiers: B415-B423 of Table 3B. In some embodiments, the PEgRNA comprises a PBS comprising a sequence selected from the group consisting of SEQ ID Nos: 1535-1541 of Table 3B. In some embodiments, the PEgRNA comprises a PBS comprising a sequence selected from the group consisting of sequences: TATCCGGG and TATCCGGGT. In some embodiments, the PEgRNA comprises an editing template comprising a sequence selected from the group consisting of SEQ identifiers: C1265-C1318 of Table 3C. In some embodiments, the prime editing composition further comprises a ngRNA that comprises a spacer comprising a sequence selected from the group consisting of SEQ identifiers: D170-D195, D204-D207, and D313 of Table 3D. In some embodiments, the PEgRNA comprises an editing template comprising a sequence selected from the group consisting of SEQ ID Nos: 5291-5344 of Table 3C. In some embodiments, the prime editing composition further comprises a ngRNA that comprises a spacer comprising a sequence selected from the group consisting of SEQ ID Nos: 19803-19828, 19837-19840, and 19946 of Table 3D.

In some embodiments, the PEgRNA comprises a spacer comprising a sequence selected from the group consisting of SEQ identifiers: A98-A99 of Table 3A. In some embodiments, the PEgRNA comprises a spacer comprising a sequence selected from the group consisting of SEQ ID Nos: 446-447 of Table 3A. In some embodiments, the PEgRNA comprises a PBS comprising a sequence selected from the group consisting of identifiers: B424-B432 of Table 3B. In some embodiments, the PEgRNA comprises a PBS comprising a sequence selected from the group consisting of SEQ ID Nos: 1542-1548 of Table 3B. In some embodiments, the PEgRNA comprises a PBS comprising a sequence selected from the group consisting of sequences: GCGATTTA and GCGATTTAT. In some embodiments, the PEgRNA comprises an editing template comprising a sequence selected from the group consisting of SEQ identifiers: C1319-C1349 of Table 3C. In some embodiments, the prime editing composition further comprises a ngRNA that comprises a spacer comprising a sequence selected from the group consisting of SEQ identifiers: D136-D154, and D158-D168 of Table 3D. In some embodiments, the PEgRNA comprises an editing template comprising a sequence selected from the group consisting of SEQ ID Nos: 5345-5375 of Table 3C. In some embodiments, the prime editing composition further comprises a ngRNA that comprises a spacer comprising a sequence selected from the group consisting of SEQ ID Nos: 19769-19787, and 19791-19801 of Table 3D.

In some embodiments, the PEgRNA comprises a spacer comprising the sequence of SEQ identifier: A100 of table 3A. In some embodiments, the PEgRNA comprises a spacer comprising the sequence of SEQ ID No: 448 of table 3A. In some embodiments, the PEgRNA comprises a PBS comprising a sequence selected from the group consisting of SEQ identifiers: B433-B441 of Table 3B. In some embodiments, the PEgRNA comprises a PBS comprising a sequence selected from the group consisting of SEQ ID Nos: 1549-1555 of Table 3B. In some embodiments, the PEgRNA comprises a PBS comprising a sequence selected from the group consisting of sequences: TCTTTATT and TCTTTATTG. In some embodiments, the PEgRNA comprises an editing template comprising the sequence selected of identifier: C1350 of table 3C. In some embodiments, the prime editing composition further comprises a ngRNA that comprises a spacer comprising a sequence selected from the group consisting of SEQ identifiers: D137-D157 of Table 3D. In some embodiments, the PEgRNA comprises an editing template comprising the sequence selected of SEQ ID No: 5376 of table 3C. In some embodiments, the prime editing composition further comprises a ngRNA that comprises a spacer comprising a sequence selected from the group consisting of SEQ ID Nos: 19770-19790 of Table 3D.

In some embodiments, the PEgRNA comprises a spacer comprising a sequence selected from the group consisting of SEQ identifiers: A101-A104 of Table 3A. In some embodiments, the PEgRNA comprises a spacer comprising a sequence selected from the group consisting of SEQ ID Nos: 449-452 of Table 3A. In some embodiments, the PEgRNA comprises a PBS comprising a sequence selected from the group consisting of SEQ identifiers: B442-B450 of Table 3B. In some embodiments, the PEgRNA comprises a PBS comprising a sequence selected from the group consisting of SEQ ID Nos: 1556-1562 of Table 3B. In some embodiments, the PEgRNA comprises a PBS comprising a sequence selected from the group consisting of sequences: ATCCGGGT and ATCCGGGTC. In some embodiments, the PEgRNA comprises an editing template comprising a sequence selected from the group consisting of SEQ identifiers: C1351-C1402 of Table 3C. In some embodiments, the prime editing composition further comprises a ngRNA that comprises a spacer comprising a sequence selected from the group consisting of SEQ identifiers: D176, D177, D251-D259, D261, D262, D264-D272, D274-D280, D283-D288, and D322-D325 of Table 3D. In some embodiments, the PEgRNA comprises an editing template comprising a sequence selected from the group consisting of SEQ ID Nos: 5377-5428 of Table 3C. In some embodiments, the prime editing composition further comprises a ngRNA that comprises a spacer comprising a sequence selected from the group consisting of SEQ ID Nos: 19809, 19810, 19884-19892, 19894, 19895, 19897-19905, 19907-19913, 19916-19921, and 19955-19958 of Table 3D.

In some embodiments, the PEgRNA comprises a spacer comprising the sequence of SEQ identifier: A50 of Table 3A. In some embodiments, the PEgRNA comprises a spacer comprising the sequence of SEQ ID No: 398 of Table 3A. In some embodiments, the PEgRNA comprises a PBS comprising a sequence selected from the group consisting of SEQ identifiers: B217-B225 of Table 3B. In some embodiments, the PEgRNA comprises a PBS comprising a sequence selected from the group consisting of SEQ ID Nos: 1381-1387 of Table 3B. In some embodiments, the PEgRNA comprises a PBS comprising a sequence selected from the group consisting of sequences: GCTTATGC and GCTTATGCC. In some embodiments, the PEgRNA comprises an editing template comprising a sequence selected from the group consisting of SEQ identifiers: C638-C650 of Table 3C. In some embodiments, the prime editing composition further comprises a ngRNA that comprises a spacer comprising a sequence selected from the group consisting of SEQ identifiers: D136-D157 of Table 3D. In some embodiments, the PEgRNA comprises an editing template comprising a sequence selected from the group consisting of SEQ ID Nos: 4664-4676 of Table 3C. In some embodiments, the prime editing composition further comprises a ngRNA that comprises a spacer comprising a sequence selected from the group consisting of SEQ ID Nos: 19769-19790 of Table 3D.

In some embodiments, the PEgRNA comprises a spacer comprising the of SEQ identifier: A51 of Table 3A. In some embodiments, the PEgRNA comprises a spacer comprising the of SEQ ID No: 399 of Table 3A. In some embodiments, the PEgRNA comprises a PBS comprising a sequence selected from the group consisting of SEQ identifiers: B226-B234 of Table 3B. In some embodiments, the PEgRNA comprises a PBS comprising a sequence selected from the group consisting of SEQ ID Nos: 1388-1394 of Table 3B. In some embodiments, the PEgRNA comprises a PBS comprising a sequence selected from the group consisting of sequences: TATCTAGG and TATCTAGGC. In some embodiments, the PEgRNA comprises an editing template comprising a sequence selected from the group consisting of SEQ identifiers: C651-C676 of Table 3C. In some embodiments, the prime editing composition further comprises a ngRNA that comprises a spacer comprising a sequence selected from the group consisting of SEQ identifiers: D136-D155, and D158-D 161 of Table 3D. In some embodiments, the PEgRNA comprises an editing template comprising a sequence selected from the group consisting of SEQ ID Nos: 4677-4702 of Table 3C. In some embodiments, the prime editing composition further comprises a ngRNA that comprises a spacer comprising a sequence selected from the group consisting of SEQ ID Nos: 19769-19788, and 19791-19794 of Table 3D.

In some embodiments, the PEgRNA comprises a spacer comprising a sequence selected from the group consisting of SEQ identifiers: A52-A53 of Table 3A. In some embodiments, the PEgRNA comprises a spacer comprising a sequence selected from the group consisting of SEQ ID Nos: 400-401 of Table 3A. In some embodiments, the PEgRNA comprises a PBS comprising a sequence selected from the group consisting of SEQ identifiers: B235-B243 of Table 3B. In some embodiments, the PEgRNA comprises a PBS comprising a sequence selected from the group consisting of SEQ ID Nos: 1395-1401 of Table 3B. In some embodiments, the PEgRNA comprises a PBS comprising a sequence selected from the group consisting of sequences: CATAGGCT and CATAGGCTT. In some embodiments, the PEgRNA comprises an editing template comprising a sequence selected from the group consisting of SEQ identifiers: C677-C694 of Table 3C. In some embodiments, the prime editing composition further comprises a ngRNA that comprises a spacer comprising a sequence selected from the group consisting of SEQ identifiers: D136-D157, and D161-D169 of Table 3D. In some embodiments, the PEgRNA comprises an editing template comprising a sequence selected from the group consisting of SEQ ID Nos: 4703-4720 of Table 3C. In some embodiments, the prime editing composition further comprises a ngRNA that comprises a spacer comprising a sequence selected from the group consisting of SEQ ID Nos: 19769-19790, and 19794-19802 of Table 3D.

In some embodiments, the PEgRNA comprises a spacer comprising a sequence selected from the group consisting of SEQ identifiers: A54-A55 of Table 3A. In some embodiments, the PEgRNA comprises a spacer comprising a sequence selected from the group consisting of SEQ ID Nos: 402-403 of Table 3A. In some embodiments, the PEgRNA comprises a PBS comprising a sequence selected from the group consisting of SEQ identifiers: B244-B252 of Table 3B. In some embodiments, the PEgRNA comprises a PBS comprising a sequence selected from the group consisting of SEQ ID Nos: 1402-1408 of Table 3B. In some embodiments, the PEgRNA comprises a PBS comprising a sequence selected from the group consisting of sequences: CATAGGAA and CATAGGAAG. In some embodiments, the PEgRNA comprises an editing template comprising a sequence selected from the group consisting of SEQ identifiers: C695-C730 of Table 3C. In some embodiments, the prime editing composition further comprises a ngRNA that comprises a spacer comprising a sequence selected from the group consisting of SEQ identifiers: D170-D195 of Table 3D. In some embodiments, the PEgRNA comprises an editing template comprising a sequence selected from the group consisting of SEQ ID Nos: 4721-4756 of Table 3C. In some embodiments, the prime editing composition further comprises a ngRNA that comprises a spacer comprising a sequence selected from the group consisting of SEQ ID Nos: 19803-19828 of Table 3D.

In some embodiments, the PEgRNA comprises a spacer comprising a sequence selected from the group consisting of SEQ identifiers: A56-A57 of Table 3A. In some embodiments, the PEgRNA comprises a spacer comprising a sequence selected from the group consisting of SEQ ID Nos: 404-405 of Table 3A. In some embodiments, the PEgRNA comprises a PBS comprising a sequence selected from the group consisting of SEQ identifiers: B253-B261 of Table 3B. In some embodiments, the PEgRNA comprises a PBS comprising a sequence selected from the group consisting of SEQ ID Nos: 1409-1415 of Table 3B. In some embodiments, the PEgRNA comprises a PBS comprising a sequence selected from the group consisting of sequences: TTGTTATC and TTGTTATCC. In some embodiments, the PEgRNA comprises an editing template comprising a sequence selected from the group consisting of SEQ identifiers: C731-C761 of Table 3C. In some embodiments, the prime editing composition further comprises a ngRNA that comprises a spacer comprising a sequence selected from the group consisting of SEQ identifiers: D172-D190, and D196-D207 of Table 3D. In some embodiments, the PEgRNA comprises an editing template comprising a sequence selected from the group consisting of SEQ ID Nos: 4757-4787 of Table 3C. In some embodiments, the prime editing composition further comprises a ngRNA that comprises a spacer comprising a sequence selected from the group consisting of SEQ ID Nos: 19805-19824, and 19829-19840 of Table 3D.

In some embodiments, the PEgRNA comprises a spacer comprising a sequence selected from the group consisting of SEQ identifiers: A58, A59, A105, and A106 of Table 3A. In some embodiments, the PEgRNA comprises a spacer comprising a sequence selected from the group consisting of SEQ identifiers: 406, 407, 453, and 454 of Table 3A. In some embodiments, the PEgRNA comprises a PBS comprising a sequence selected from the group consisting of SEQ identifiers: B262-B270 of Table 3B. In some embodiments, the PEgRNA comprises a PBS comprising a sequence selected from the group consisting of SEQ ID Nos: 1416-1422 of Table 3B. In some embodiments, the PEgRNA comprises a PBS comprising a sequence selected from the group consisting of sequences: CGATTTAT and CGATTTATC. In some embodiments, the PEgRNA comprises an editing template comprising a sequence selected from the group consisting of SEQ identifiers: C762-C823 of Table 3C. In some embodiments, the prime editing composition further comprises a ngRNA that comprises a spacer comprising a sequence selected from the group consisting of SEQ identifiers: D145, D208-D226, D301, D302, D304, and D305 of Table 3D. In some embodiments, the PEgRNA comprises an editing template comprising a sequence selected from the group consisting of SEQ ID Nos: 4788-4849 of Table 3C. In some embodiments, the prime editing composition further comprises a ngRNA that comprises a spacer comprising a sequence selected from the group consisting of SEQ ID Nos: 19778, 19841-19859, 19934, 19935, 19937, and 19938 of Table 3D.

In some embodiments, the PEgRNA comprises a spacer comprising a sequence selected from the group consisting of SEQ identifiers: A60-A61 of Table 3A. In some embodiments, the PEgRNA comprises a spacer comprising a sequence selected from the group consisting of SEQ ID Nos: 408-409 of Table 3A. In some embodiments, the PEgRNA comprises a PBS comprising a sequence selected from the group consisting of SEQ identifiers: B271-B279 of Table 3B. In some embodiments, the PEgRNA comprises a PBS comprising a sequence selected from the group consisting of SEQ ID Nos: 1423-1429 of Table 3B. In some embodiments, the PEgRNA comprises a PBS comprising a sequence selected from the group consisting of sequences: CCTTCTCT and CCTTCTCTT. In some embodiments, the PEgRNA comprises an editing template comprising a sequence selected from the group consisting of SEQ identifiers: C824-C835 of Table 3C. In some embodiments, the prime editing composition further comprises a ngRNA that comprises a spacer comprising a sequence selected from the group consisting of SEQ identifiers: D210-D215, D219-D224, and D227-D230 of Table 3D. In some embodiments, the PEgRNA comprises an editing template comprising a sequence selected from the group consisting of SEQ ID Nos: 4850-4861 of Table 3C. In some embodiments, the prime editing composition further comprises a ngRNA that comprises a spacer comprising a sequence selected from the group consisting of SEQ ID Nos: 19843-19848, 19852-19857, and 19860-19863 of Table 3D.

In some embodiments, the PEgRNA comprises a spacer comprising the sequence of SEQ identifier: A62 of Table 3A. In some embodiments, the PEgRNA comprises a spacer comprising the sequence of SEQ ID No: 410 of Table 3A. In some embodiments, the PEgRNA comprises a PBS comprising a sequence selected from the group consisting of SEQ identifiers: B280-B288 of Table 3B. In some embodiments, the PEgRNA comprises a PBS comprising a sequence selected from the group consisting of SEQ ID Nos: 1430-1436 of Table 3B. In some embodiments, the PEgRNA comprises a PBS comprising a sequence selected from the group consisting of sequences: GCCTTCTC and GCCTTCTCT. In some embodiments, the PEgRNA comprises an editing template comprising a sequence selected from the group consisting of SEQ identifiers: C836-C842 of Table 3C. In some embodiments, the prime editing composition further comprises a ngRNA that comprises a spacer comprising a sequence selected from the group consisting of SEQ identifiers: D137-D157 of Table 3D. In some embodiments, the PEgRNA comprises an editing template comprising a sequence selected from the group consisting of SEQ ID Nos: 4862-4868 of Table 3C. In some embodiments, the prime editing composition further comprises a ngRNA that comprises a spacer comprising a sequence selected from the group consisting of SEQ ID Nos: 19770-19790 of Table 3D.

In some embodiments, the PEgRNA comprises a spacer comprising a sequence selected from the group consisting of SEQ identifiers: A67-A68 of Table 3A. In some embodiments, the PEgRNA comprises a spacer comprising a sequence selected from the group consisting of SEQ ID Nos: 415-416 of Table 3A. In some embodiments, the PEgRNA comprises a PBS comprising a sequence selected from the group consisting of SEQ identifiers: B298-B306 of Table 3B. In some embodiments, the PEgRNA comprises a PBS comprising a sequence selected from the group consisting of SEQ ID Nos: 1444-1450 of Table 3B. In some embodiments, the PEgRNA comprises a PBS comprising a sequence selected from the group consisting of sequences: TTATCCGG and TTATCCGGG. In some embodiments, the PEgRNA comprises an editing template comprising a sequence selected from the group consisting of SEQ identifiers: C874-C901 of Table 3C. In some embodiments, the prime editing composition further comprises a ngRNA that comprises a spacer comprising a sequence selected from the group consisting of SEQ identifiers: D170-D190, D196-D207, D248, and D249 of Table 3D. In some embodiments, the PEgRNA comprises an editing template comprising a sequence selected from the group consisting of SEQ ID Nos: 4900-4927 of Table 3C. In some embodiments, the prime editing composition further comprises a ngRNA that comprises a spacer comprising a sequence selected from the group consisting of SEQ ID Nos: 19803-19823, 19829-19840, 19881, and 19882 of Table 3D.

In some embodiments, the PEgRNA comprises a spacer comprising a sequence selected from the group consisting of SEQ identifiers: A69-A70 of Table 3A. In some embodiments, the PEgRNA comprises a spacer comprising a sequence selected from the group consisting of SEQ ID Nos: 417-418 of Table 3A. In some embodiments, the PEgRNA comprises a PBS comprising a sequence selected from the group consisting of SEQ identifiers: B307-B315 of Table 3B. In some embodiments, the PEgRNA comprises a PBS comprising a sequence selected from the group consisting of SEQ ID Nos: 1451-1457 of Table 3B. In some embodiments, the PEgRNA comprises a PBS comprising a sequence selected from the group consisting of sequences: TTTATCTA and TTTATCTAG. In some embodiments, the PEgRNA comprises an editing template comprising a sequence selected from the group consisting of SEQ identifiers: C902-C929 of Table 3C. In some embodiments, the prime editing composition further comprises a ngRNA that comprises a spacer comprising a sequence selected from the group consisting of SEQ identifiers: D136-D155, and D158-D169 of Table 3D. In some embodiments, the PEgRNA comprises an editing template comprising a sequence selected from the group consisting of SEQ ID Nos: 4928-4955 of Table 3C. In some embodiments, the prime editing composition further comprises a ngRNA that comprises a spacer comprising a sequence selected from the group consisting of SEQ ID Nos: 19769-19788, and 19791-19802 of Table 3D.

In some embodiments, the PEgRNA comprises a spacer comprising the sequence of SEQ identifier: A73 of Table 3A. In some embodiments, the PEgRNA comprises a spacer comprising the sequence of SEQ ID No: 421 of Table 3A. In some embodiments, the PEgRNA comprises a PBS comprising a sequence selected from the group consisting of SEQ identifiers: B325-B333 of Table 3B. In some embodiments, the PEgRNA comprises a PBS comprising a sequence selected from the group consisting of SEQ ID Nos: 1465-1471 of Table 3B. In some embodiments, the PEgRNA comprises a PBS comprising a sequence selected from the group consisting of sequences: TATCGCGA and TATCGCGA. In some embodiments, the PEgRNA comprises an editing template comprising a sequence selected from the group consisting of SEQ identifiers: C961-C991 of Table 3C. In some embodiments, the prime editing composition further comprises a ngRNA that comprises a spacer comprising a sequence selected from the group consisting of SEQ identifiers: D136-D154, D158-D161 and D250 of Table 3D. In some embodiments, the PEgRNA comprises an editing template comprising a sequence selected from the group consisting of SEQ ID Nos: 4987-5017 of Table 3C. In some embodiments, the prime editing composition further comprises a ngRNA that comprises a spacer comprising a sequence selected from the group consisting of SEQ ID Nos: 19769-19787, 19791-19794 and 19883 of Table 3D.

In some embodiments, the PEgRNA comprises a spacer comprising a sequence selected from the group consisting of SEQ identifiers: A74-A77 of Table 3A. In some embodiments, the PEgRNA comprises a spacer comprising a sequence selected from the group consisting of SEQ ID Nos: 422-425 of Table 3A. In some embodiments, the PEgRNA comprises a PBS comprising a sequence selected from the group consisting of SEQ identifiers: B334-B342 of Table 3B. In some embodiments, the PEgRNA comprises a PBS comprising a sequence selected from the group consisting of SEQ ID Nos: 1472-1478 of Table 3B. In some embodiments, the PEgRNA comprises a PBS comprising a sequence selected from the group consisting of sequences: GTTATCCG and GTTATCCGG. In some embodiments, the PEgRNA comprises an editing template comprising a sequence selected from the group consisting of SEQ identifiers: C992-C1049 of Table 3C. In some embodiments, the prime editing composition further comprises a ngRNA that comprises a spacer comprising a sequence selected from the group consisting of SEQ identifiers: D176, D177, D207, and D251-D290 of Table 3D. In some embodiments, the PEgRNA comprises an editing template comprising a sequence selected from the group consisting of SEQ ID Nos: 5018-5075 of Table 3C. In some embodiments, the prime editing composition further comprises a ngRNA that comprises a spacer comprising a sequence selected from the group consisting of SEQ ID Nos: 19809, 19810, 19840, and 19884-19923 of Table 3D.

In some embodiments, the PEgRNA comprises a spacer comprising a sequence selected from the group consisting of SEQ identifiers: A78-A79 of Table 3A. In some embodiments, the PEgRNA comprises a spacer comprising a sequence selected from the group consisting of SEQ ID Nos: 426-427 of Table 3A. In some embodiments, the PEgRNA comprises a PBS comprising a sequence selected from the group consisting of SEQ identifiers: B343-B351 of Table 3B. In some embodiments, the PEgRNA comprises a PBS comprising a sequence selected from the group consisting of SEQ ID Nos: 1479-1485 of Table 3B. In some embodiments, the PEgRNA comprises a PBS comprising a sequence selected from the group consisting of sequences: TTCTTCCC and TTCTTCCCA. In some embodiments, the PEgRNA comprises an editing template comprising a sequence selected from the group consisting of SEQ identifiers: C1050-C1055 of Table 3C. In some embodiments, the prime editing composition further comprises a ngRNA that comprises a spacer comprising a sequence selected from the group consisting of SEQ identifiers: D251-D258, D264-D271, and D291-D298 of Table 3D. In some embodiments, the PEgRNA comprises an editing template comprising a sequence selected from the group consisting of SEQ ID Nos: 5076-5081 of Table 3C. In some embodiments, the prime editing composition further comprises a ngRNA that comprises a spacer comprising a sequence selected from the group consisting of SEQ ID Nos: 19884-19891, 19897-19904, and 19924-19931 of Table 3D.

In some embodiments, the PEgRNA comprises a spacer comprising the sequence of SEQ identifiers: A80 of Table 3A. In some embodiments, the PEgRNA comprises a spacer comprising the sequence of SEQ ID Nos: 428 of Table 3A. In some embodiments, the PEgRNA comprises a PBS comprising a sequence selected from the group consisting of SEQ identifiers: B352-B360 of Table 3B. In some embodiments, the PEgRNA comprises a PBS comprising a sequence selected from the group consisting of SEQ ID Nos: 1486-1492 of Table 3B. In some embodiments, the PEgRNA comprises a PBS comprising a sequence selected from the group consisting of sequences: ATTCTTCC and ATTCTTCCC. In some embodiments, the PEgRNA comprises an editing template comprising a sequence selected from the group consisting of SEQ identifiers: C1056-C1059 of Table 3C. In some embodiments, the prime editing composition further comprises a ngRNA that comprises a spacer comprising a sequence selected from the group consisting of SEQ identifiers: D170-D187 of Table 3D. In some embodiments, the PEgRNA comprises an editing template comprising a sequence selected from the group consisting of SEQ ID Nos: 5082-5085 of Table 3C. In some embodiments, the prime editing composition further comprises a ngRNA that comprises a spacer comprising a sequence selected from the group consisting of SEQ ID Nos: 19803-19820 of Table 3D.

In some embodiments, the PEgRNA comprises a spacer comprising a sequence selected from the group consisting of SEQ identifiers: A81-A82 of Table 3A. In some embodiments, the PEgRNA comprises a spacer comprising a sequence selected from the group consisting of SEQ ID Nos: 429-430 of Table 3A. In some embodiments, the PEgRNA comprises a PBS comprising a sequence selected from the group consisting of SEQ identifiers: B361-B369 of Table 3B. In some embodiments, the PEgRNA comprises a PBS comprising a sequence selected from the group consisting of SEQ ID Nos: 1493-1499 of Table 3B. In some embodiments, the PEgRNA comprises a PBS comprising a sequence selected from the group consisting of sequences: GAAGCTAT and GAAGCTATG. In some embodiments, the PEgRNA comprises an editing template comprising a sequence selected from the group consisting of SEQ identifiers: C1060-C1072 of Table 3. In some embodiments, the prime editing composition further comprises a ngRNA that comprises a spacer comprising a sequence selected from the group consisting of SEQ identifiers: D170-D188, D196-D202, D248, and D249 of Table 3D. In some embodiments, the PEgRNA comprises an editing template comprising a sequence selected from the group consisting of SEQ ID Nos: 5086-5098 of Table 3. In some embodiments, the prime editing composition further comprises a ngRNA that comprises a spacer comprising a sequence selected from the group consisting of SEQ ID Nos: 19803-19821, 19829-19835, 19881, and 19882 of Table 3D.

In some embodiments, the PEgRNA comprises a spacer comprising a sequence selected from the group consisting of SEQ identifiers: A83-A84 of Table 3A. In some embodiments, the PEgRNA comprises a spacer comprising a sequence selected from the group consisting of SEQ ID Nos: 431-432 of Table 3A. In some embodiments, the PEgRNA comprises a PBS comprising a sequence selected from the group consisting of SEQ identifiers: B370-B378 of Table 3B. In some embodiments, the PEgRNA comprises a PBS comprising a sequence selected from the group consisting of SEQ ID Nos: 1500-1506 of Table 3B. In some embodiments, the PEgRNA comprises a PBS comprising a sequence selected from the group consisting of sequences: ATAGGCTT and ATAGGCTTA. In some embodiments, the PEgRNA comprises an editing template comprising a sequence selected from the group consisting of SEQ identifiers: C1073-C1106 of Table 3C. In some embodiments, the prime editing composition further comprises a ngRNA that comprises a spacer comprising a sequence selected from the group consisting of SEQ identifiers: D210-D215, D219-D224, and D227-D230 of Table 3D. In some embodiments, the PEgRNA comprises an editing template comprising a sequence selected from the group consisting of SEQ ID Nos: 5099-5132 of Table 3C. In some embodiments, the prime editing composition further comprises a ngRNA that comprises a spacer comprising a sequence selected from the group consisting of SEQ ID Nos: 19843-19848, 19852-19857, and 19860-19863 of Table 3D.

In some embodiments, the PEgRNA comprises a spacer comprising a sequence selected from the group consisting of SEQ identifiers: A85-A87 of Table 3A. In some embodiments, the PEgRNA comprises a spacer comprising a sequence selected from the group consisting of SEQ ID Nos: 433-435 of Table 3A. In some embodiments, the PEgRNA comprises a PBS comprising a sequence selected from the group consisting of SEQ identifiers: B379-B387 of Table 3B. In some embodiments, the PEgRNA comprises a PBS comprising a sequence selected from the group consisting of SEQ ID Nos: 1507-1513 of Table 3B. In some embodiments, the PEgRNA comprises a PBS comprising a sequence selected from the group consisting of sequences: ATAGGAAG and ATAGGAAGC. In some embodiments, the PEgRNA comprises an editing template comprising a sequence selected from the group consisting of SEQ identifiers: C1107-C1123 of Table 3C. In some embodiments, the prime editing composition further comprises a ngRNA that comprises a spacer comprising a sequence selected from the group consisting of SEQ identifiers: D176, D177, D251, D254, D255, D262, D275-D279, and D283-D300 of Table 3D. In some embodiments, the PEgRNA comprises an editing template comprising a sequence selected from the group consisting of SEQ ID Nos: 5133-5149 of Table 3C. In some embodiments, the prime editing composition further comprises a ngRNA that comprises a spacer comprising a sequence selected from the group consisting of SEQ ID Nos: 19809, 19810, 19884, 19887, 19888, D262, 19908-19912, and 19916-19933 of Table 3D.

In some embodiments, the PEgRNA comprises a spacer comprising a sequence selected from the group consisting of SEQ identifiers: A88-A89 of Table 3A. In some embodiments, the PEgRNA comprises a spacer comprising a sequence selected from the group consisting of SEQ ID Nos: 436-437 of Table 3A. In some embodiments, the PEgRNA comprises a PBS comprising a sequence selected from the group consisting of SEQ identifiers: B388-B396 of Table 3B. In some embodiments, the PEgRNA comprises a PBS comprising a sequence selected from the group consisting of SEQ ID Nos: 1514-1520 of Table 3B. In some embodiments, the PEgRNA comprises a PBS comprising a sequence selected from the group consisting of sequences: CGCGATTT and CGCGATTTA. In some embodiments, the PEgRNA comprises an editing template comprising a sequence selected from the group consisting of SEQ identifiers: C1124-C1185 of Table 3C. In some embodiments, the prime editing composition further comprises a ngRNA that comprises a spacer comprising a sequence selected from the group consisting of SEQ identifiers: D209-D216, D219-D226, D303 and D306 of Table 3D. In some embodiments, the PEgRNA comprises an editing template comprising a sequence selected from the group consisting of SEQ ID Nos: 5150-5211 of Table 3C. In some embodiments, the prime editing composition further comprises a ngRNA that comprises a spacer comprising a sequence selected from the group consisting of SEQ ID Nos: 19842-19849, 19852-19859, 19936 and 19939 of Table 3D.

In some embodiments, the PEgRNA comprises a spacer comprising the sequence of SEQ identifier: A64 of Table 3A. In some embodiments, the PEgRNA comprises a spacer comprising the sequence of SEQ ID No: 412 of Table 3A. In some embodiments, the PEgRNA comprises a PBS comprising a sequence selected from the group consisting of SEQ identifiers: B289-B297 of Table 3B. In some embodiments, the PEgRNA comprises a PBS comprising a sequence selected from the group consisting of SEQ ID Nos: 1437-1443 of Table 3B. In some embodiments, the PEgRNA comprises a PBS comprising a sequence selected from the group consisting of sequences: TCGCGATT and TCGCGATTT. In some embodiments, the PEgRNA comprises an editing template comprising a sequence selected from the group consisting of SEQ identifiers: C843-C873 of Table 3C. In some embodiments, the prime editing composition further comprises a ngRNA that comprises a spacer comprising a sequence selected from the group consisting of SEQ identifiers: D136-D154, and D158-D161 of Table 3D. In some embodiments, the PEgRNA comprises an editing template comprising a sequence selected from the group consisting of SEQ ID Nos: 4869-4899 of Table 3C. In some embodiments, the prime editing composition further comprises a ngRNA that comprises a spacer comprising a sequence selected from the group consisting of SEQ ID Nos: 19769-19787, and 19791-19794 of Table 3D.

In some embodiments, the PEgRNA comprises a spacer comprising a sequence selected from the group consisting of SEQ identifiers: A92-A93 of Table 3A. In some embodiments, the PEgRNA comprises a spacer comprising a sequence selected from the group consisting of SEQ ID Nos: 440-441 of Table 3A. In some embodiments, the PEgRNA comprises a PBS comprising a sequence selected from the group consisting of SEQ identifiers: B397-B405 of Table 3B. In some embodiments, the PEgRNA comprises a PBS comprising a sequence selected from the group consisting of SEQ ID Nos: 1521-1527 of Table 3B. In some embodiments, the PEgRNA comprises a PBS comprising a sequence selected from the group consisting of sequences: TGTTATCC and TGTTATCCG. In some embodiments, the PEgRNA comprises an editing template comprising a sequence selected from the group consisting of SEQ identifiers: C1186-C1245 of Table 3C. In some embodiments, the prime editing composition further comprises a ngRNA that comprises a spacer comprising a sequence selected from the group consisting of SEQ identifiers: D171-D195, D204-D207, and D307-D315 of Table 3D. In some embodiments, the PEgRNA comprises an editing template comprising a sequence selected from the group consisting of SEQ ID Nos: 5212-5271 of Table 3C. In some embodiments, the prime editing composition further comprises a ngRNA that comprises a spacer comprising a sequence selected from the group consisting of SEQ ID Nos: 19804-19828, 19837-19840, and 19940-19948 of Table 3D.

In some embodiments, the PEgRNA comprises a spacer comprising a sequence selected from the group consisting of SEQ identifiers: A94-A95 of Table 3A. In some embodiments, the PEgRNA comprises a spacer comprising a sequence selected from the group consisting of SEQ ID Nos: 442-443 of Table 3A. In some embodiments, the PEgRNA comprises a PBS comprising a sequence selected from the group consisting of SEQ identifiers: B406-B414 of Table 3B. In some embodiments, the PEgRNA comprises a PBS comprising a sequence selected from the group consisting of SEQ ID Nos: 1528-1534 of Table 3B. In some embodiments, the PEgRNA comprises a PBS comprising a sequence selected from the group consisting of sequences: TCATAGGA and TCATAGGAA. In some embodiments, the PEgRNA comprises an editing template comprising a sequence selected from the group consisting of SEQ identifiers: C1246-C1264 of Table 3C. In some embodiments, the prime editing composition further comprises a ngRNA that comprises a spacer comprising a sequence selected from the group consisting of SEQ identifiers: D170-D190, D196-D204, D248, and D249 of Table 3D. In some embodiments, the PEgRNA comprises an editing template comprising a sequence selected from the group consisting of SEQ ID Nos: 5272-5290 of Table 3C. In some embodiments, the prime editing composition further comprises a ngRNA that comprises a spacer comprising a sequence selected from the group consisting of SEQ ID Nos: 19803-19823, 19829-19837, 19881, and 19882 of Table 3D.

In some embodiments, the PEgRNA comprises a spacer comprising a sequence selected from the group consisting of SEQ identifiers: A96-A97 of Table 3A. In some embodiments, the PEgRNA comprises a spacer comprising a sequence selected from the group consisting of SEQ ID Nos: 444-445 of Table 3A. In some embodiments, the PEgRNA comprises a PBS comprising a sequence selected from the group consisting of SEQ identifiers: B415-B423 of Table 3B. In some embodiments, the PEgRNA comprises a PBS comprising a sequence selected from the group consisting of SEQ ID Nos: 1535-1541 of Table 3B. In some embodiments, the PEgRNA comprises a PBS comprising a sequence selected from the group consisting of sequences: TATCCGGG and TATCCGGGT. In some embodiments, the PEgRNA comprises an editing template comprising a sequence selected from the group consisting of SEQ identifiers: C1265-C1318 of Table 3C. In some embodiments, the prime editing composition further comprises a ngRNA that comprises a spacer comprising a sequence selected from the group consisting of SEQ identifiers: D170-D195, D204-D207, and D313-D321 of Table 3D. In some embodiments, the PEgRNA comprises an editing template comprising a sequence selected from the group consisting of SEQ ID Nos: 5291-5344 of Table 3C. In some embodiments, the prime editing composition further comprises a ngRNA that comprises a spacer comprising a sequence selected from the group consisting of SEQ ID Nos: 19803-19828, 19837-19840, and 19946-19954 of Table 3D.

In some embodiments, the PEgRNA comprises a spacer comprising a sequence selected from the group consisting of SEQ identifiers: A98-A99 of Table 3A. In some embodiments, the PEgRNA comprises a spacer comprising a sequence selected from the group consisting of SEQ ID Nos: 446-447 of Table 3A. In some embodiments, the PEgRNA comprises a PBS comprising a sequence selected from the group consisting of SEQ identifiers: B424-B432 of Table 3B. In some embodiments, the PEgRNA comprises a PBS comprising a sequence selected from the group consisting of SEQ ID Nos: 1542-1548 of Table 3B. In some embodiments, the PEgRNA comprises a PBS comprising a sequence selected from the group consisting of sequences: GCGATTTA and GCGATTTAT. In some embodiments, the PEgRNA comprises an editing template comprising a sequence selected from the group consisting of SEQ identifiers: C1319-C1349 of Table 3C. In some embodiments, the prime editing composition further comprises a ngRNA that comprises a spacer comprising a sequence selected from the group consisting of SEQ identifiers: D136-D154 and D158-D168 of Table 3D. In some embodiments, the PEgRNA comprises an editing template comprising a sequence selected from the group consisting of SEQ ID Nos: 5345-C 5375 of Table 3C. In some embodiments, the prime editing composition further comprises a ngRNA that comprises a spacer comprising a sequence selected from the group consisting of SEQ ID Nos: 19769-19787 and 19791-19801 of Table 3D.

In some embodiments, the PEgRNA comprises a spacer comprising the sequence of SEQ identifier: A100 of Table 3A. In some embodiments, the PEgRNA comprises a spacer comprising the sequence of SEQ ID No: 448 of Table 3A. In some embodiments, the PEgRNA comprises a PBS comprising a sequence selected from the group consisting of SEQ identifiers: B433-B441 of Table 3B. In some embodiments, the PEgRNA comprises a PBS comprising a sequence selected from the group consisting of SEQ ID Nos: 1549-1555 of Table 3B. In some embodiments, the PEgRNA comprises a PBS comprising a sequence selected from the group consisting of sequences: TCTTTATT and TCTTTATTG. In some embodiments, the PEgRNA comprises an editing template comprising the sequence of SEQ identifier: C1350 of Table 3C. In some embodiments, the prime editing composition further comprises a ngRNA that comprises a spacer comprising a sequence selected from the group consisting of SEQ identifiers: D137-D157 of Table 3D. In some embodiments, the PEgRNA comprises an editing template comprising the sequence of SEQ ID No: 5376 of Table 3C. In some embodiments, the prime editing composition further comprises a ngRNA that comprises a spacer comprising a sequence selected from the group consisting of SEQ ID Nos: 19770-19790 of Table 3D.

In some embodiments, the PEgRNA comprises a spacer comprising a sequence selected from the group consisting of SEQ identifiers: A101-A104 of Table 3A. In some embodiments, the PEgRNA comprises a spacer comprising a sequence selected from the group consisting of SEQ ID Nos: 449-452 of Table 3A. In some embodiments, the PEgRNA comprises a PBS comprising a sequence selected from the group consisting of SEQ identifiers: B442-B450 of Table 3B. In some embodiments, the PEgRNA comprises a PBS comprising a sequence selected from the group consisting of SEQ ID Nos: 1556-1562 of Table 3B. In some embodiments, the PEgRNA comprises a PBS comprising a sequence selected from the group consisting of sequences: ATCCGGGT and ATCCGGGTC. In some embodiments, the PEgRNA comprises an editing template comprising a sequence selected from the group consisting of SEQ identifiers: C1351-C1402 of Table 3C. In some embodiments, the prime editing composition further comprises a ngRNA that comprises a spacer comprising a sequence selected from the group consisting of SEQ identifiers: D176, D177, D251-D259, D261-D262, D264-D280, D283-D288, and D322-D325 of Table 3D. In some embodiments, the PEgRNA comprises an editing template comprising a sequence selected from the group consisting of SEQ ID Nos: 5377-5428 of Table 3C. In some embodiments, the prime editing composition further comprises a ngRNA that comprises a spacer comprising a sequence selected from the group consisting of SEQ ID Nos: 19809, D177, 19884-19892, 19894-19895, 19897-19913, 19916-19921, and 19955-19958 of Table 3D.

In some embodiments, the PEgRNA comprises a spacer comprising the sequence of SEQ identifier: A50 of Table 3A. In some embodiments, the PEgRNA comprises a spacer comprising the sequence of SEQ ID No: 398 of Table 3A. In some embodiments, the PEgRNA comprises a PBS comprising a sequence selected from the group consisting of SEQ identifiers: B217-B225 of Table 3B. In some embodiments, the PEgRNA comprises a PBS comprising a sequence selected from the group consisting of SEQ ID Nos: 1381-1387 of Table 3B. In some embodiments, the PEgRNA comprises a PBS comprising a sequence selected from the group consisting of sequences: GCTTATGC and GCTTATGCC. In some embodiments, the PEgRNA comprises an editing template comprising a sequence selected from the group consisting of SEQ identifiers: C638-C650 of Table 3C. In some embodiments, the prime editing composition further comprises a ngRNA that comprises a spacer comprising a sequence selected from the group consisting of SEQ identifiers: D136-D157 of Table 3D. In some embodiments, the PEgRNA comprises an editing template comprising a sequence selected from the group consisting of SEQ ID Nos: 4664-4676 of Table 3C. In some embodiments, the prime editing composition further comprises a ngRNA that comprises a spacer comprising a sequence selected from the group consisting of SEQ ID Nos: 19769-19790 of Table 3D.

In some embodiments, the PEgRNA comprises a spacer comprising the of SEQ identifier: A51 of Table 3A. In some embodiments, the PEgRNA comprises a spacer comprising the of SEQ ID No: 399 of Table 3A. In some embodiments, the PEgRNA comprises a PBS comprising a sequence selected from the group consisting of SEQ identifiers: B226-B234 of Table 3B. In some embodiments, the PEgRNA comprises a PBS comprising a sequence selected from the group consisting of SEQ ID Nos: 1388-1394 of Table 3B. In some embodiments, the PEgRNA comprises a PBS comprising a sequence selected from the group consisting of sequence: TATCTAGG and TATCTAGGC. In some embodiments, the PEgRNA comprises an editing template comprising a sequence selected from the group consisting of SEQ identifiers: C651-C676 of Table 3C. In some embodiments, the prime editing composition further comprises a ngRNA that comprises a spacer comprising a sequence selected from the group consisting of SEQ identifiers: D136-D155 and D158-D161 of Table 3D. In some embodiments, the PEgRNA comprises an editing template comprising a sequence selected from the group consisting of SEQ ID Nos: 4677-4702 of Table 3C. In some embodiments, the prime editing composition further comprises a ngRNA that comprises a spacer comprising a sequence selected from the group consisting of SEQ ID Nos: D136-D155 and D158-D161 of Table 3D.

In some embodiments, the PEgRNA comprises a spacer comprising a sequence selected from the group consisting of SEQ identifiers: A52-A53 of Table 3A. In some embodiments, the PEgRNA comprises a spacer comprising a sequence selected from the group consisting of SEQ ID Nos: 400-401 of Table 3A. In some embodiments, the PEgRNA comprises a PBS comprising a sequence selected from the group consisting of SEQ identifiers: B235-B243 of Table 3B. In some embodiments, the PEgRNA comprises a PBS comprising a sequence selected from the group consisting of SEQ ID Nos: 1395-1401 of Table 3B. In some embodiments, the PEgRNA comprises a PBS comprising a sequence selected from the group consisting of sequences: CATAGGCT and CATAGGCTT. In some embodiments, the PEgRNA comprises an editing template comprising a sequence selected from the group consisting of SEQ identifiers: C677-C694 of Table 3C. In some embodiments, the prime editing composition further comprises a ngRNA that comprises a spacer comprising a sequence selected from the group consisting of SEQ identifiers: D136-D157, and D161-D169 of Table 3D. In some embodiments, the PEgRNA comprises an editing template comprising a sequence selected from the group consisting of SEQ ID Nos: 4703-4720 of Table 3C. In some embodiments, the prime editing composition further comprises a ngRNA that comprises a spacer comprising a sequence selected from the group consisting of SEQ ID Nos: 19769-19790, and 19794-19802 of Table 3D.

In some embodiments, the PEgRNA comprises a spacer comprising a sequence selected from the group consisting of SEQ identifiers: A54-A55 of Table 3A. In some embodiments, the PEgRNA comprises a spacer comprising a sequence selected from the group consisting of SEQ ID Nos: 402-403 of Table 3A. In some embodiments, the PEgRNA comprises a PBS comprising a sequence selected from the group consisting of SEQ identifiers: B244-B252 of Table 3B. In some embodiments, the PEgRNA comprises a PBS comprising a sequence selected from the group consisting of SEQ ID Nos: 1402-1408 of Table 3B. In some embodiments, the PEgRNA comprises a PBS comprising a sequence selected from the group consisting of sequences: CATAGGAA and CATAGGAAG. In some embodiments, the PEgRNA comprises an editing template comprising a sequence selected from the group consisting of SEQ identifiers: C695-C730 of Table 3C. In some embodiments, the prime editing composition further comprises a ngRNA that comprises a spacer comprising a sequence selected from the group consisting of SEQ identifiers: D170-D195 of Table 3D. In some embodiments, the PEgRNA comprises an editing template comprising a sequence selected from the group consisting of SEQ ID Nos: 4721-4756 of Table 3C. In some embodiments, the prime editing composition further comprises a ngRNA that comprises a spacer comprising a sequence selected from the group consisting of SEQ ID Nos: 19803-19828 of Table 3D.

In some embodiments, the PEgRNA comprises a spacer comprising a sequence selected from the group consisting of SEQ identifiers: A56-A57 of Table 3A. In some embodiments, the PEgRNA comprises a spacer comprising a sequence selected from the group consisting of SEQ ID Nos: 404-405 of Table 3A. In some embodiments, the PEgRNA comprises a PBS comprising a sequence selected from the group consisting of SEQ identifiers: B253-B261 of Table 3B. In some embodiments, the PEgRNA comprises a PBS comprising a sequence selected from the group consisting of SEQ ID Nos: 1409-1415 of Table 3B. In some embodiments, the PEgRNA comprises a PBS comprising a sequence selected from the group consisting of sequences: TTGTTATC and TTGTTATCC. In some embodiments, the PEgRNA comprises an editing template comprising a sequence selected from the group consisting of SEQ identifiers: C731-C761 of Table 3C. In some embodiments, the prime editing composition further comprises a ngRNA that comprises a spacer comprising a sequence selected from the group consisting of SEQ identifiers: D172-D190, and D196-D207 of Table 3D. In some embodiments, the PEgRNA comprises an editing template comprising a sequence selected from the group consisting of SEQ ID Nos: 4757-4787 of Table 3C. In some embodiments, the prime editing composition further comprises a ngRNA that comprises a spacer comprising a sequence selected from the group consisting of SEQ ID Nos: 19805-19823, and 19829-19840 of Table 3D.

In some embodiments, the PEgRNA comprises a spacer comprising a sequence selected from the group consisting of SEQ identifiers: A58-A59 of Table 3A. In some embodiments, the PEgRNA comprises a spacer comprising a sequence selected from the group consisting of SEQ ID Nos: 406-407 of Table 3A. In some embodiments, the PEgRNA comprises a PBS comprising a sequence selected from the group consisting of SEQ identifiers: B262-B270 of Table 3B. In some embodiments, the PEgRNA comprises a PBS comprising a sequence selected from the group consisting of SEQ ID Nos: 1417-1422 of Table 3B. In some embodiments, the PEgRNA comprises a PBS comprising a sequence selected from the group consisting of sequences: CGATTTAT and CGATTTATC. In some embodiments, the PEgRNA comprises an editing template comprising a sequence selected from the group consisting of SEQ identifiers: C762-C823 of Table 3. In some embodiments, the prime editing composition further comprises a ngRNA that comprises a spacer comprising a sequence selected from the group consisting of SEQ identifiers: D208-D226 of Table 3D. In some embodiments, the PEgRNA comprises an editing template comprising a sequence selected from the group consisting of SEQ ID Nos: 4788-4849 of Table 3. In some embodiments, the prime editing composition further comprises a ngRNA that comprises a spacer comprising a sequence selected from the group consisting of SEQ ID Nos: 19841-19859 of Table 3D.

In some embodiments, the PEgRNA comprises a spacer comprising a sequence selected from the group consisting of SEQ identifiers: A60-A61 of Table 3A. In some embodiments, the PEgRNA comprises a spacer comprising a sequence selected from the group consisting of SEQ ID Nos: 408-409 of Table 3A. In some embodiments, the PEgRNA comprises a PBS comprising a sequence selected from the group consisting of SEQ identifiers: B271-B279 of Table 3B. In some embodiments, the PEgRNA comprises a PBS comprising a sequence selected from the group consisting of SEQ ID Nos: 1423-1429 of Table 3B. In some embodiments, the PEgRNA comprises a PBS comprising a sequence selected from the group consisting of sequences: CCTTCTCT and CCTTCTCTT. In some embodiments, the PEgRNA comprises an editing template comprising a sequence selected from the group consisting of SEQ identifiers: C824-C835 of Table 3C. In some embodiments, the prime editing composition further comprises a ngRNA that comprises a spacer comprising a sequence selected from the group consisting of SEQ identifiers: D210-D215, D219-D224, and D227-D230 of Table 3D. In some embodiments, the PEgRNA comprises an editing template comprising a sequence selected from the group consisting of SEQ ID Nos: 4850-4861 of Table 3C. In some embodiments, the prime editing composition further comprises a ngRNA that comprises a spacer comprising a sequence selected from the group consisting of SEQ ID Nos: 19843-19848, 19852-19857, and 19860-19863 of Table 3D.

In some embodiments, the PEgRNA comprises a spacer comprising the sequence of SEQ identifier: A62 of Table 3A. In some embodiments, the PEgRNA comprises a spacer comprising the sequence of SEQ ID No: 410 of Table 3A. In some embodiments, the PEgRNA comprises a PBS comprising a sequence selected from the group consisting of SEQ identifiers: B280-B288 of Table 3B. In some embodiments, the PEgRNA comprises a PBS comprising a sequence selected from the group consisting of SEQ ID Nos: 1430-1436 of Table 3B. In some embodiments, the PEgRNA comprises a PBS comprising a sequence selected from the group consisting of sequences: GCCTTCTC and GCCTTCTCT. In some embodiments, the PEgRNA comprises an editing template comprising a sequence selected from the group consisting of SEQ identifiers: C836-C842 of Table 3C. In some embodiments, the prime editing composition further comprises a ngRNA that comprises a spacer comprising a sequence selected from the group consisting of SEQ identifiers: D137-D157 of Table 3D. In some embodiments, the PEgRNA comprises an editing template comprising a sequence selected from the group consisting of SEQ ID Nos: 4862-4868 of Table 3C. In some embodiments, the prime editing composition further comprises a ngRNA that comprises a spacer comprising a sequence selected from the group consisting of SEQ ID Nos: 19770-19790 of Table 3D.

In some embodiments, the PEgRNA comprises a spacer comprising a sequence selected from the group consisting of SEQ identifiers: A63-A64 of Table 3A. In some embodiments, the PEgRNA comprises a spacer comprising a sequence selected from the group consisting of SEQ ID Nos: 411-412 of Table 3A. In some embodiments, the PEgRNA comprises a PBS comprising a sequence selected from the group consisting of SEQ identifiers: B289-B297 of Table 3B. In some embodiments, the PEgRNA comprises a PBS comprising a sequence selected from the group consisting of SEQ ID Nos: 1437-1443 of Table 3B. In some embodiments, the PEgRNA comprises a PBS comprising a sequence selected from the group consisting of SEQ ID Nos: TCGCGATT and TCGCGATTT. In some embodiments, the PEgRNA comprises an editing template comprising a sequence selected from the group consisting of SEQ identifiers: C843-C873 of Table 3C. In some embodiments, the prime editing composition further comprises a ngRNA that comprises a spacer comprising a sequence selected from the group consisting of SEQ identifiers: D136-D154, and D158-D168 of Table 3D. In some embodiments, the PEgRNA comprises an editing template comprising a sequence selected from the group consisting of SEQ ID Nos: 4869-4899 of Table 3C. In some embodiments, the prime editing composition further comprises a ngRNA that comprises a spacer comprising a sequence selected from the group consisting of SEQ ID Nos: 19769-19787, and 19791-19801 of Table 3D.

In some embodiments, the PEgRNA comprises a spacer comprising a sequence selected from the group consisting of SEQ identifiers: A67-A68 of Table 3A. In some embodiments, the PEgRNA comprises a spacer comprising a sequence selected from the group consisting of SEQ ID Nos: 415-416 of Table 3A. In some embodiments, the PEgRNA comprises a PBS comprising a sequence selected from the group consisting of SEQ identifiers: B298-B306 of Table 3B. In some embodiments, the PEgRNA comprises a PBS comprising a sequence selected from the group consisting of SEQ ID Nos: 1444-1450 of Table 3B. In some embodiments, the PEgRNA comprises a PBS comprising a sequence selected from the group consisting of sequences: TTATCCGG and TTATCCGGG. In some embodiments, the PEgRNA comprises an editing template comprising a sequence selected from the group consisting of SEQ identifiers: C874-C901 of Table 3C. In some embodiments, the prime editing composition further comprises a ngRNA that comprises a spacer comprising a sequence selected from the group consisting of SEQ identifiers: D170-D190, D196-D207, D248, and D249 of Table 3D. In some embodiments, the PEgRNA comprises an editing template comprising a sequence selected from the group consisting of SEQ ID Nos: 4900-4927 of Table 3C. In some embodiments, the prime editing composition further comprises a ngRNA that comprises a spacer comprising a sequence selected from the group consisting of SEQ ID Nos: 19803-19823, 19829-19840, 19881, and 19882 of Table 3D.

In some embodiments, the PEgRNA comprises a spacer comprising a sequence selected from the group consisting of SEQ identifiers: A69-A70 of Table 3A. In some embodiments, the PEgRNA comprises a spacer comprising a sequence selected from the group consisting of SEQ ID Nos: 417-418 of Table 3A. In some embodiments, the PEgRNA comprises a PBS comprising a sequence selected from the group consisting of SEQ identifers: B307-B315 of Table 3B. In some embodiments, the PEgRNA comprises a PBS comprising a sequence selected from the group consisting of SEQ ID Nos: 1451-1457 of Table 3B. In some embodiments, the PEgRNA comprises a PBS comprising a sequence selected from the group consisting of sequences: TTTATCTA and TTTATCTAG. In some embodiments, the PEgRNA comprises an editing template comprising a sequence selected from the group consisting of SEQ identifiers: C902-C929 of Table 3C. In some embodiments, the prime editing composition further comprises a ngRNA that comprises a spacer comprising a sequence selected from the group consisting of SEQ identifiers: D136-D155, and D158-D169 of Table 3D. In some embodiments, the PEgRNA comprises an editing template comprising a sequence selected from the group consisting of SEQ ID Nos: 4928-4955 of Table 3C. In some embodiments, the prime editing composition further comprises a ngRNA that comprises a spacer comprising a sequence selected from the group consisting of SEQ ID Nos: 19769-19788, and 19791-19802 of Table 3D.

In some embodiments, the PEgRNA comprises a spacer comprising a sequence selected from the group consisting of SEQ ID Nos: A71-A72 of Table 3A. In some embodiments, the PEgRNA comprises a spacer comprising a sequence selected from the group consisting of SEQ ID Nos: 419-420 of Table 3A. In some embodiments, the PEgRNA comprises a PBS comprising a sequence selected from the group consisting of SEQ identifers: B316-B324 of Table 3B. In some embodiments, the PEgRNA comprises a PBS comprising a sequence selected from the group consisting of SEQ ID Nos: 1458-1464 of Table 3B. In some embodiments, the PEgRNA comprises a PBS comprising a sequence selected from the group consisting of sequences: ATCGCGAT and ATCGCGATT. In some embodiments, the PEgRNA comprises an editing template comprising a sequence selected from the group consisting of SEQ identifiers: C930-C960 of Table 3C. In some embodiments, the prime editing composition further comprises a ngRNA that comprises a spacer comprising a sequence selected from the group consisting of SEQ identifiers: D209-D216, and D219-D226 of Table 3D. In some embodiments, the PEgRNA comprises an editing template comprising a sequence selected from the group consisting of SEQ ID Nos: 4956-4986 of Table 3C. In some embodiments, the prime editing composition further comprises a ngRNA that comprises a spacer comprising a sequence selected from the group consisting of SEQ ID Nos: 19842-19849, and 19852-19859 of Table 3D.

In some embodiments, the PEgRNA comprises a spacer comprising the sequence of SEQ ID No: A73 of Table 3A. In some embodiments, the PEgRNA comprises a spacer comprising the sequence of SEQ ID No: 421 of Table 3A. In some embodiments, the PEgRNA comprises a PBS comprising a sequence selected from the group consisting of SEQ identifiers: B325-B333 of Table 3B. In some embodiments, the PEgRNA comprises a PBS comprising a sequence selected from the group consisting of SEQ ID Nos: 1465-1471 of Table 3B. In some embodiments, the PEgRNA comprises a PBS comprising a sequence selected from the group consisting of sequences: TATCGCGA and TATCGCGA. In some embodiments, the PEgRNA comprises an editing template comprising a sequence selected from the group consisting of SEQ identifiers: C961-C991 of Table 3C. In some embodiments, the prime editing composition further comprises a ngRNA that comprises a spacer comprising a sequence selected from the group consisting of SEQ identifiers: D136-D154, D158-D161, and D250 of Table 3D. In some embodiments, the PEgRNA comprises an editing template comprising a sequence selected from the group consisting of SEQ ID Nos: 4987-5017 of Table 3C. In some embodiments, the prime editing composition further comprises a ngRNA that comprises a spacer comprising a sequence selected from the group consisting of SEQ ID Nos: 19769-19787, 19791-19794, and 19883 of Table 3D.

In some embodiments, the PEgRNA comprises a spacer comprising a sequence selected from the group consisting of SEQ ID Nos: A74-A77 of Table 3A. In some embodiments, the PEgRNA comprises a spacer comprising a sequence selected from the group consisting of SEQ ID Nos: 422-425 of Table 3A. In some embodiments, the PEgRNA comprises a PBS comprising a sequence selected from the group consisting of SEQ identifiers: B334-B342 of Table 3B. In some embodiments, the PEgRNA comprises a PBS comprising a sequence selected from the group consisting of SEQ ID Nos: 1472-1478 of Table 3B. In some embodiments, the PEgRNA comprises a PBS comprising a sequence selected from the group consisting of sequences: GTTATCCG and GTTATCCGG. In some embodiments, the PEgRNA comprises an editing template comprising a sequence selected from the group consisting of SEQ identifiers: C992-C1049 of Table 3C. In some embodiments, the prime editing composition further comprises a ngRNA that comprises a spacer comprising a sequence selected from the group consisting of SEQ identifiers: D176, D177, D207, D251-D290 of Table 3D. In some embodiments, the PEgRNA comprises an editing template comprising a sequence selected from the group consisting of SEQ ID Nos: 5018-5075 of Table 3C. In some embodiments, the prime editing composition further comprises a ngRNA that comprises a spacer comprising a sequence selected from the group consisting of SEQ ID Nos: 19809, 19810, 19840, 19884-19923 of Table 3D.

In some embodiments, the PEgRNA comprises a spacer comprising a sequence selected from the group consisting of SEQ ID Nos: A78-A79 of Table 3A. In some embodiments, the PEgRNA comprises a spacer comprising a sequence selected from the group consisting of SEQ ID Nos: 426-427 of Table 3A. In some embodiments, the PEgRNA comprises a PBS comprising a sequence selected from the group consisting of SEQ identifiers: B343-B351 of Table 3B. In some embodiments, the PEgRNA comprises a PBS comprising a sequence selected from the group consisting of SEQ ID Nos: 1479-1485 of Table 3B. In some embodiments, the PEgRNA comprises a PBS comprising a sequence selected from the group consisting of SEQ ID Nos: B343-B351 of Table 3B. In some embodiments, the PEgRNA comprises an editing template comprising a sequence selected from the group consisting of SEQ identifiers: C1050-C1055 of Table 3C. In some embodiments, the prime editing composition further comprises a ngRNA that comprises a spacer comprising a sequence selected from the group consisting of SEQ identifiers: D251-D258, D264-D271, and D291-D298 of Table 3D. In some embodiments, the PEgRNA comprises an editing template comprising a sequence selected from the group consisting of SEQ ID Nos: 5076-5081 of Table 3C. In some embodiments, the prime editing composition further comprises a ngRNA that comprises a spacer comprising a sequence selected from the group consisting of SEQ ID Nos: 19884-19891, 19897-19904, and 19924-19931 of Table 3D.

In some embodiments, the PEgRNA comprises a spacer comprising the sequence of SEQ ID No: A80 of Table 3A. In some embodiments, the PEgRNA comprises a spacer comprising the sequence of SEQ ID No: 428 of Table 3A. In some embodiments, the PEgRNA comprises a PBS comprising a sequence selected from the group consisting of SEQ identifiers: B352-B360 of Table 3B. In some embodiments, the PEgRNA comprises a PBS comprising a sequence selected from the group consisting of SEQ ID Nos: 1486-1492 of Table 3B. In some embodiments, the PEgRNA comprises a PBS comprising a sequence selected from the group consisting of sequences: ATTCTTCC and ATTCTTCCC. In some embodiments, the PEgRNA comprises an editing template comprising a sequence selected from the group consisting of SEQ identifiers: C1056-C1059 of Table 3C. In some embodiments, the prime editing composition further comprises a ngRNA that comprises a spacer comprising a sequence selected from the group consisting of SEQ identifiers: D170-D187 of Table 3D. In some embodiments, the PEgRNA comprises an editing template comprising a sequence selected from the group consisting of SEQ ID Nos: 5082-5085 of Table 3C. In some embodiments, the prime editing composition further comprises a ngRNA that comprises a spacer comprising a sequence selected from the group consisting of SEQ ID Nos: 19803-19820 of Table 3D.

In some embodiments, the PEgRNA comprises a spacer comprising a sequence selected from the group consisting of SEQ ID Nos: A81-A82 of Table 3A. In some embodiments, the PEgRNA comprises a spacer comprising a sequence selected from the group consisting of SEQ ID Nos: 429-430 of Table 3A. In some embodiments, the PEgRNA comprises a PBS comprising a sequence selected from the group consisting of SEQ identifiers: B361-B369 of Table 3B. In some embodiments, the PEgRNA comprises a PBS comprising a sequence selected from the group consisting of SEQ ID Nos: 1493-1499 of Table 3B. In some embodiments, the PEgRNA comprises a PBS comprising a sequence selected from the group consisting of sequences: GAAGCTAT and GAAGCTATG. In some embodiments, the PEgRNA comprises an editing template comprising a sequence selected from the group consisting of SEQ identifiers: C1060-C1072 of Table 3C. In some embodiments, the prime editing composition further comprises a ngRNA that comprises a spacer comprising a sequence selected from the group consisting of SEQ identifiers: D170-D188, D196-D202, D248, and D249 of Table 3D. In some embodiments, the PEgRNA comprises an editing template comprising a sequence selected from the group consisting of SEQ ID Nos: 5086-5098 of Table 3C. In some embodiments, the prime editing composition further comprises a ngRNA that comprises a spacer comprising a sequence selected from the group consisting of SEQ ID Nos: 19803-19821, 19829-19835, 19881, and 19882 of Table 3D.

In some embodiments, the PEgRNA comprises a spacer comprising a sequence selected from the group consisting of SEQ ID Nos: A83-A84 of Table 3A. In some embodiments, the PEgRNA comprises a spacer comprising a sequence selected from the group consisting of SEQ ID Nos: 431-432 of Table 3A. In some embodiments, the PEgRNA comprises a PBS comprising a sequence selected from the group consisting of SEQ identifiers: B370-B378 of Table 3B. In some embodiments, the PEgRNA comprises a PBS comprising a sequence selected from the group consisting of SEQ ID Nos: 1500-1506 of Table 3B. In some embodiments, the PEgRNA comprises a PBS comprising a sequence selected from the group consisting of sequences: ATAGGCTT and ATAGGCTTA. In some embodiments, the PEgRNA comprises an editing template comprising a sequence selected from the group consisting of SEQ identifiers: C1073-C1106 of Table 3C. In some embodiments, the prime editing composition further comprises a ngRNA that comprises a spacer comprising a sequence selected from the group consisting of SEQ identifiers: D210-D215, D219-D224, and D227-D230 of Table 3D. In some embodiments, the PEgRNA comprises an editing template comprising a sequence selected from the group consisting of SEQ ID Nos: 5099-5132 of Table 3C. In some embodiments, the prime editing composition further comprises a ngRNA that comprises a spacer comprising a sequence selected from the group consisting of SEQ ID Nos: 19843-19848, 19852-19857, and 19860-19863 of Table 3D.

In some embodiments, the PEgRNA comprises a spacer comprising a sequence selected from the group consisting of SEQ ID Nos: A85-A87 of Table 3A. In some embodiments, the PEgRNA comprises a spacer comprising a sequence selected from the group consisting of SEQ ID Nos: 433-435 of Table 3A. In some embodiments, the PEgRNA comprises a PBS comprising a sequence selected from the group consisting of SEQ identifiers: B379-B387 of Table 3B. In some embodiments, the PEgRNA comprises a PBS comprising a sequence selected from the group consisting of SEQ ID Nos: 1507-1513 of Table 3B. In some embodiments, the PEgRNA comprises a PBS comprising a sequence selected from the group consisting of sequences: ATAGGAAG and ATAGGAAGC. In some embodiments, the PEgRNA comprises an editing template comprising a sequence selected from the group consisting of SEQ identifiers: C1107-C1123 of Table 3C. In some embodiments, the prime editing composition further comprises a ngRNA that comprises a spacer comprising a sequence selected from the group consisting of SEQ identifiers: D176, D177, D251, D254, D255, D262, D275-D279, D283-D287, D299, and D300 of Table 3D. In some embodiments, the PEgRNA comprises an editing template comprising a sequence selected from the group consisting of SEQ ID Nos: 5133-5149 of Table 3C. In some embodiments, the prime editing composition further comprises a ngRNA that comprises a spacer comprising a sequence selected from the group consisting of SEQ ID Nos: 19809, 19810, 19884, 19887, 19888, 19895, 19908-19912, 19916-19920, 19932, and 19933 of Table 3D.

In some embodiments, the PEgRNA comprises a spacer comprising a sequence selected from the group consisting of SEQ ID Nos: A88-A91 of Table 3A. In some embodiments, the PEgRNA comprises a spacer comprising a sequence selected from the group consisting of SEQ ID Nos: 436-439 of Table 3A. In some embodiments, the PEgRNA comprises a PBS comprising a sequence selected from the group consisting of SEQ identifiers: B388-B396 of Table 3B. In some embodiments, the PEgRNA comprises a PBS comprising a sequence selected from the group consisting of SEQ ID Nos: 1514-1520 of Table 3B. In some embodiments, the PEgRNA comprises a PBS comprising a sequence selected from the group consisting of sequences: CGCGATTT and CGCGATTTA. In some embodiments, the PEgRNA comprises an editing template comprising a sequence selected from the group consisting of SEQ identifiers: C1124-C1185 of Table 3C. In some embodiments, the prime editing composition further comprises a ngRNA that comprises a spacer comprising a sequence selected from the group consisting of SEQ identifiers: D145, D209-D216, D219-D226, and D301-D306 of Table 3D. In some embodiments, the PEgRNA comprises an editing template comprising a sequence selected from the group consisting of SEQ ID Nos: 5150-5211 of Table 3C. In some embodiments, the prime editing composition further comprises a ngRNA that comprises a spacer comprising a sequence selected from the group consisting of SEQ ID Nos: 19778, 19842-19849, 19852-19859, and 19934-19939 of Table 3D.

In some embodiments, the PEgRNA comprises a spacer comprising a sequence selected from the group consisting of SEQ ID Nos: A92-A93 of Table 3A. In some embodiments, the PEgRNA comprises a spacer comprising a sequence selected from the group consisting of SEQ ID Nos: 440-441 of Table 3A. In some embodiments, the PEgRNA comprises a PBS comprising a sequence selected from the group consisting of SEQ identifiers: B397-B405 of Table 3B. In some embodiments, the PEgRNA comprises a PBS comprising a sequence selected from the group consisting of SEQ ID Nos: 1521-1527 of Table 3B. In some embodiments, the PEgRNA comprises a PBS comprising a sequence selected from the group consisting of sequences: TGTTATCC and TGTTATCCG. In some embodiments, the PEgRNA comprises an editing template comprising a sequence selected from the group consisting of SEQ identifiers: C1186-C1245 of Table 3C. In some embodiments, the prime editing composition further comprises a ngRNA that comprises a spacer comprising a sequence selected from the group consisting of SEQ identifiers: D171-D195, D204-D207, and D307-D315 of Table 3D. In some embodiments, the PEgRNA comprises an editing template comprising a sequence selected from the group consisting of SEQ ID Nos: 5212-5271 of Table 3C. In some embodiments, the prime editing composition further comprises a ngRNA that comprises a spacer comprising a sequence selected from the group consisting of SEQ ID Nos: 19804-19828, 19837-19840, and 19940-19948 of Table 3D.

In some embodiments, the PEgRNA comprises a spacer comprising a sequence selected from the group consisting of SEQ ID Nos: A94-A95 of Table 3A. In some embodiments, the PEgRNA comprises a spacer comprising a sequence selected from the group consisting of SEQ ID Nos: 442-443 of Table 3A. In some embodiments, the PEgRNA comprises a PBS comprising a sequence selected from the group consisting of SEQ identifiers: B406-B414 of Table 3B. In some embodiments, the PEgRNA comprises a PBS comprising a sequence selected from the group consisting of SEQ ID Nos: 1528-1534 of Table 3B. In some embodiments, the PEgRNA comprises a PBS comprising a sequence selected from the group consisting of SEQ ID Nos: TCATAGGA and TCATAGGAA. In some embodiments, the PEgRNA comprises an editing template comprising a sequence selected from the group consisting of SEQ identifiers: C1246-C1264 of Table 3C. In some embodiments, the prime editing composition further comprises a ngRNA that comprises a spacer comprising a sequence selected from the group consisting of SEQ identifiers: D170-D190, D196-D204, D248, and D249 of Table 3D. In some embodiments, the PEgRNA comprises an editing template comprising a sequence selected from the group consisting of SEQ ID Nos: 5272-5290 of Table 3C. In some embodiments, the prime editing composition further comprises a ngRNA that comprises a spacer comprising a sequence selected from the group consisting of SEQ ID Nos: 19803-19823, 19829-19837, 19881, and 19882 of Table 3D.

In some embodiments, the PEgRNA comprises a spacer comprising a sequence selected from the group consisting of SEQ ID Nos: A96-A97 of Table 3A. In some embodiments, the PEgRNA comprises a spacer comprising a sequence selected from the group consisting of SEQ ID Nos: 444-445 of Table 3A. In some embodiments, the PEgRNA comprises a PBS comprising a sequence selected from the group consisting of SEQ identifiers: B415-B423 of Table 3B. In some embodiments, the PEgRNA comprises a PBS comprising a sequence selected from the group consisting of SEQ ID Nos: 1535-1541 of Table 3B. In some embodiments, the PEgRNA comprises a PBS comprising a sequence selected from the group consisting of sequences: TATCCGGG and TATCCGGGT. In some embodiments, the PEgRNA comprises an editing template comprising a sequence selected from the group consisting of SEQ identifiers: C1265-C1318 of Table 3C. In some embodiments, the prime editing composition further comprises a ngRNA that comprises a spacer comprising a sequence selected from the group consisting of SEQ identifiers: D170-D195, D205-D207, and D313-D321 of Table 3D. In some embodiments, the PEgRNA comprises an editing template comprising a sequence selected from the group consisting of SEQ ID Nos: 5291-5344 of Table 3C. In some embodiments, the prime editing composition further comprises a ngRNA that comprises a spacer comprising a sequence selected from the group consisting of SEQ ID Nos: 19803-19828, 19838-19840, and 19946-19954 of Table 3D.

In some embodiments, the PEgRNA comprises a spacer comprising a sequence selected from the group consisting of SEQ ID Nos: A98-A99 of Table 3A. In some embodiments, the PEgRNA comprises a spacer comprising a sequence selected from the group consisting of SEQ ID Nos: 446-447 of Table 3A. In some embodiments, the PEgRNA comprises a PBS comprising a sequence selected from the group consisting of SEQ identifiers: B424-B432 of Table 3B. In some embodiments, the PEgRNA comprises a PBS comprising a sequence selected from the group consisting of SEQ ID Nos: 1542-1548 of Table 3B. In some embodiments, the PEgRNA comprises a PBS comprising a sequence selected from the group consisting of sequences: GCGATTTA and GCGATTTAT. In some embodiments, the PEgRNA comprises an editing template comprising a sequence selected from the group consisting of SEQ identifiers: C1319-C1349 of Table 3C. In some embodiments, the prime editing composition further comprises a ngRNA that comprises a spacer comprising a sequence selected from the group consisting of SEQ identifiers: D136-D154, and D158-D168 of Table 3D. In some embodiments, the PEgRNA comprises an editing template comprising a sequence selected from the group consisting of SEQ ID Nos: 5345-5375 of Table 3C. In some embodiments, the prime editing composition further comprises a ngRNA that comprises a spacer comprising a sequence selected from the group consisting of SEQ ID Nos: 19769-19787, and 19791-19801 of Table 3D.

In some embodiments, the PEgRNA comprises a spacer comprising the sequence of SEQ ID No: A100 of Table 3A. In some embodiments, the PEgRNA comprises a spacer comprising the sequence of SEQ ID No: 448 of Table 3A. In some embodiments, the PEgRNA comprises a PBS comprising a sequence selected from the group consisting of SEQ identifiers: B433-B441 of Table 3B. In some embodiments, the PEgRNA comprises a PBS comprising a sequence selected from the group consisting of SEQ ID Nos: 1549-1555 of Table 3B. In some embodiments, the PEgRNA comprises a PBS comprising a sequence selected from the group consisting of sequences: TCTTTATT and TCTTTATTG. In some embodiments, the PEgRNA comprises an editing template comprising the sequence of SEQ identifier: C1350. In some embodiments, the prime editing composition further comprises a ngRNA that comprises a spacer comprising a sequence selected from the group consisting of SEQ identifiers: D137-D157 of Table 3D. In some embodiments, the PEgRNA comprises an editing template comprising the sequence of SEQ ID No: 5376. In some embodiments, the prime editing composition further comprises a ngRNA that comprises a spacer comprising a sequence selected from the group consisting of SEQ ID Nos: 19770-19790 of Table 3D.

In some embodiments, the PEgRNA comprises a spacer comprising a sequence selected from the group consisting of SEQ ID Nos: A101-A104 of Table 3A. In some embodiments, the PEgRNA comprises a spacer comprising a sequence selected from the group consisting of SEQ ID Nos: 449-452 of Table 3A. In some embodiments, the PEgRNA comprises a PBS comprising a sequence selected from the group consisting of SEQ identifiers: B442-B450 of Table 3B. In some embodiments, the PEgRNA comprises a PBS comprising a sequence selected from the group consisting of SEQ ID Nos: 1556-1562 of Table 3B. In some embodiments, the PEgRNA comprises a PBS comprising a sequence selected from the group consisting of sequences: ATCCGGGT and ATCCGGGTC. In some embodiments, the PEgRNA comprises an editing template comprising a sequence selected from the group consisting of SEQ identifiers: C1351-C1402 of Table 3C. In some embodiments, the prime editing composition further comprises a ngRNA that comprises a spacer comprising a sequence selected from the group consisting of SEQ identifiers: D176, D177, D251-D272, D274-D280, D283-D288, and D322-D325 of Table 3D. In some embodiments, the PEgRNA comprises an editing template comprising a sequence selected from the group consisting of SEQ ID Nos: 5377-5428 of Table 3C. In some embodiments, the prime editing composition further comprises a ngRNA that comprises a spacer comprising a sequence selected from the group consisting of SEQ ID Nos: 19809, 19810, 19884-19905, 19907-19913, 19916-19921, and 19955-19958 of Table 3D.

In some embodiments, the editing target sequence is in exon 8 of the CFTR gene.

In some embodiments, the editing target sequence or the complementary sequence on the CFTR gene thereof comprises a mutation that encodes an amino acid substitution S313*, G314R, G314V, F319V, and/or L320V relative to a wild type CFTR polypeptide set forth in SEQ ID NO: 19, wherein * refers to a non-sense mutation, and wherein the editing template encodes an intended nucleotide edit that corrects the mutation. In some embodiments, the editing target sequence or the complementary sequence on the CFTR gene thereof comprises a C→→A mutation (c.938C>A) at position 117,540,168, a T deletion mutation (c.948delT) at position 117,540,178, a G→→C mutation (c.940G>C) at position 117,540,170, a G→→T mutation (c.941G>T) at position 117,540,171, a T→→G mutation (c.955T>G) at position 117,540,185, and/or a T→→G mutation (c.958T>G) at position 117,540,188 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide. In some embodiments, the editing template comprises a wild type CFTR gene sequence. Accordingly, in some embodiments, the newly synthesized single stranded DNA encoded by the editing template is capable of correcting the c.948delT mutation, the c.938C>A mutation, the c.940G>C mutation, the c.941G>T mutation, the c.955T>G mutation, and/or the c.958T>G mutation in the target CFTR gene. In some embodiments, the newly synthesized single stranded DNA encoded by the editing template is capable of correcting all of the c.948delT mutation, the c.938C>A mutation, the c.940G>C mutation, the c.941G>T mutation, the c.955T>G mutation, and the c.958T>G mutation in the target CFTR gene. In some embodiments, two or more of the c.948delT mutation, the c.938C>A mutation, the c.940G>C mutation, the c.941G>T mutation, the c.955T>G mutation, and the c.958T>G mutation are located on the same CFTR gene. In some embodiments, the newly synthesized single stranded DNA encoded by the editing template is capable of correcting the c.948delT mutation, the c.938C>A mutation, the c.940G>C mutation, the c.941G>T mutation, the c.955T>G mutation, and the c.958T>G mutation that are located on different CFTR genes. For example, in some embodiments, a subject may comprise two copies of CFTR genes, each comprising a different mutation selected from the group consisting of the c.948delT mutation, the c.938C>A mutation, the c.940G>C mutation, the c.941G>T mutation, the c.955T>G mutation, and the c.958T>G mutation, all of which may be corrected by an editing template of a single PEgRNA. In some embodiments, the editing template is capable of correcting one or more, or all of the c.948delT mutation, the c.938C>A mutation, the c.940G>C mutation, the c.941G>T mutation, the c.955T>G mutation, and the c.958T>G mutation that are in different subjects, wherein each subject may have one or more of the mutations.

In some embodiments, the PEgRNA comprises a spacer comprising a sequence selected from the group consisting of SEQ Identifiers: A107-A110 of Table 3A. In some embodiments, the PEgRNA comprises a PBS comprising a sequence selected from the group consisting of SEQ Identifiers: B451-B459 of Table 3B. In some embodiments, the PEgRNA comprises an editing template comprising a sequence selected from the group consisting of SEQ Identifiers: C1403-C1454 of Table 3C. In some embodiments, the prime editing composition further comprises a ngRNA that comprises a spacer comprising a sequence selected from the group consisting of SEQ Identifiers: D326-D361 of Table 3D.

In some embodiments, the PEgRNA comprises a spacer comprising the sequence of SEQ Identifier: A111 of Table 3A. In some embodiments, the PEgRNA comprises a PBS comprising a sequence selected from the group consisting of SEQ Identifiers: B460-B468 of Table 3B. In some embodiments, the PEgRNA comprises an editing template comprising a sequence selected from the group consisting of SEQ Identifiers: C1455-C1460 of Table 3C. In some embodiments, the prime editing composition further comprises a ngRNA that comprises a spacer comprising a sequence selected from the group consisting of SEQ Identifiers: D362-D384 of Table 3D.

In some embodiments, the PEgRNA comprises a spacer comprising a sequence selected from the group consisting of SEQ Identifiers: A112-A113 of Table 3A. In some embodiments, the PEgRNA comprises a PBS comprising a sequence selected from the group consisting of SEQ Identifiers: B469-B477 of Table 3B. In some embodiments, the PEgRNA comprises an editing template comprising a sequence selected from the group consisting of SEQ Identifiers: C1461-C1500 of Table 3C. In some embodiments, the prime editing composition further comprises a ngRNA that comprises a spacer comprising a sequence selected from the group consisting of SEQ Identifiers: D385-D386 of Table 3D.

In some embodiments, the PEgRNA comprises a spacer comprising a sequence selected from the group consisting of SEQ Identifiers: A114-A117 of Table 3A. In some embodiments, the PEgRNA comprises a PBS comprising a sequence selected from the group consisting of SEQ Identifiers: B478-B486 of Table 3B. In some embodiments, the PEgRNA comprises an editing template comprising a sequence selected from the group consisting of SEQ Identifiers: C1501-C1522 of Table 3C. In some embodiments, the prime editing composition further comprises a ngRNA that comprises a spacer comprising a sequence selected from the group consisting of SEQ Identifiers: D362-D384, and D386-D422 of Table 3D.

In some embodiments, the PEgRNA comprises a spacer comprising a sequence selected from the group consisting of SEQ Identifiers: A118-A119 of Table 3A. In some embodiments, the PEgRNA comprises a PBS comprising a sequence selected from the group consisting of SEQ Identifiers: B487-B495 of Table 3B. In some embodiments, the PEgRNA comprises an editing template comprising a sequence selected from the group consisting of SEQ Identifiers: C1523-C1553 of Table 3C. In some embodiments, the prime editing composition further comprises a ngRNA that comprises a spacer comprising a sequence selected from the group consisting of SEQ Identifiers: D362-D379 and D423-D427 of Table 3D.

In some embodiments, the PEgRNA comprises a spacer comprising a sequence selected from the group consisting of SEQ Identifiers: A120-A121 of Table 3A. In some embodiments, the PEgRNA comprises a PBS comprising a sequence selected from the group consisting of SEQ Identifiers: B496-B504 of Table 3B. In some embodiments, the PEgRNA comprises an editing template comprising a sequence selected from the group consisting of SEQ Identifiers: C1554-C1565 of Table 3C. In some embodiments, the prime editing composition further comprises a ngRNA that comprises a spacer comprising a sequence selected from the group consisting of SEQ Identifiers: D332-D337, D349-D354, D428, and D429 of Table 3D.

In some embodiments, the PEgRNA comprises a spacer comprising the of SEQ Identifiers: A122 of Table 3A. In some embodiments, the PEgRNA comprises a PBS comprising a sequence selected from the group consisting of SEQ Identifiers: B505-B513 of Table 3B. In some embodiments, the PEgRNA comprises an editing template comprising a sequence selected from the group consisting of SEQ Identifiers: C1566-C1572 of Table 3C. In some embodiments, the prime editing composition further comprises a ngRNA that comprises a spacer comprising a sequence selected from the group consisting of SEQ Identifiers: D360, and D430-D446 of Table 3D.

In some embodiments, the PEgRNA comprises a spacer comprising a sequence selected from the group consisting of SEQ Identifiers: A123-A124 of Table 3A. In some embodiments, the PEgRNA comprises a PBS comprising a sequence selected from the group consisting of SEQ Identifiers: B514-B522 of Table 3B. In some embodiments, the PEgRNA comprises an editing template comprising a sequence selected from the group consisting of SEQ Identifiers: C1573-C1628 of Table 3C. In some embodiments, the prime editing composition further comprises a ngRNA that comprises a spacer comprising a sequence selected from the group consisting of SEQ Identifiers: D360, and D434-460 of Table 3D.

In some embodiments, the PEgRNA comprises a spacer comprising the sequence of SEQ Identifier: A125 of Table 3A. In some embodiments, the PEgRNA comprises a PBS comprising a sequence selected from the group consisting of SEQ Identifiers: B451, B523-B530 of Table 3B. In some embodiments, the PEgRNA comprises an editing template comprising a sequence selected from the group consisting of SEQ Identifiers: C1629-C1657 of Table 3C. In some embodiments, the prime editing composition further comprises a ngRNA that comprises a spacer comprising a sequence selected from the group consisting of SEQ Identifiers: D360, and D435-D446, D454-D459 of Table 3D.

In some embodiments, the PEgRNA comprises a spacer comprising a sequence selected from the group consisting of SEQ Identifiers: A126-A127 of Table 3A. In some embodiments, the PEgRNA comprises a PBS comprising a sequence selected from the group consisting of SEQ Identifiers: B531B-539 of Table 3B. In some embodiments, the PEgRNA comprises an editing template comprising a sequence selected from the group consisting of SEQ Identifiers: C1658-C1688 of Table 3C. In some embodiments, the prime editing composition further comprises a ngRNA that comprises a spacer comprising a sequence selected from the group consisting of SEQ Identifiers: D436-D446, D449, D450, D452-D459, and D462-D463 of Table 3D.

In some embodiments, the PEgRNA comprises a spacer comprising the sequence of SEQ Identifier: A128 of Table 3A. In some embodiments, the PEgRNA comprises a PBS comprising a sequence selected from the group consisting of SEQ Identifiers: B540-B548 of Table 3B. In some embodiments, the PEgRNA comprises an editing template comprising a sequence selected from the group consisting of SEQ Identifiers: C1689-C1719 of Table 3C. In some embodiments, the prime editing composition further comprises a ngRNA that comprises a spacer comprising a sequence selected from the group consisting of SEQ Identifiers: D436-D446, D453-D459, and D461-D 463 of Table 3D.

In some embodiments, the PEgRNA comprises a spacer comprising the sequence of SEQ Identifiers: A129 of Table 3A. In some embodiments, the PEgRNA comprises a PBS comprising a sequence selected from the group consisting of SEQ Identifiers: B549-B557 of Table 3B. In some embodiments, the PEgRNA comprises an editing template comprising a sequence selected from the group consisting of SEQ Identifiers: C1720-C1750 of Table 3C. In some embodiments, the prime editing composition further comprises a ngRNA that comprises a spacer comprising a sequence selected from the group consisting of SEQ Identifiers: D436-D446, D453-D459, and D461-D463 of Table 3D.

In some embodiments, the PEgRNA comprises a spacer comprising a sequence selected from the group consisting of SEQ Identifiers: A130-A131 of Table 3A. In some embodiments, the PEgRNA comprises a PBS comprising a sequence selected from the group consisting of SEQ Identifiers: B558-B566 of Table 3B. In some embodiments, the PEgRNA comprises an editing template comprising a sequence selected from the group consisting of SEQ Identifiers: C1751-C1780 of Table 3C.

In some embodiments, the prime editing composition further comprises a ngRNA that comprises a spacer comprising a sequence selected from the group consisting of SEQ Identifiers: D385, D386, D464, and D465 of Table 3D.

In some embodiments, the PEgRNA comprises a spacer comprising the sequence of SEQ Identifier: A132 of Table 3A. In some embodiments, the PEgRNA comprises a PBS comprising a sequence selected from the group consisting of SEQ Identifiers: B567-B575 of Table 3 of Table 3B. In some embodiments, the PEgRNA comprises an editing template comprising a sequence selected from the group consisting of SEQ Identifiers: C1781-C1796 of Table 3C. In some embodiments, the prime editing composition further comprises a ngRNA that comprises a spacer comprising a sequence selected from the group consisting of SEQ Identifiers: D362-D383 of Table 3D.

In some embodiments, the PEgRNA comprises a spacer comprising a sequence selected from the group consisting of SEQ Identifiers: A133-A136 of Table 3A. In some embodiments, the PEgRNA comprises a PBS comprising a sequence selected from the group consisting of SEQ Identifiers: B576-B584 of Table 3B. In some embodiments, the PEgRNA comprises an editing template comprising a sequence selected from the group consisting of SEQ Identifiers: C1797-C1816 of Table 3C. In some embodiments, the prime editing composition further comprises a ngRNA that comprises a spacer comprising a sequence selected from the group consisting of SEQ Identifiers: D376, D385-D395, D406-D412, D414-D422, D466, and D467 of Table 3D.

In some embodiments, the PEgRNA comprises a spacer comprising a sequence selected from the group consisting of SEQ Identifiers: A137-A138 of Table 3A. In some embodiments, the PEgRNA comprises a PBS comprising a sequence selected from the group consisting of SEQ Identifiers: B585-B593 of Table 3B. In some embodiments, the PEgRNA comprises an editing template comprising a sequence selected from the group consisting of SEQ Identifiers: C1817-C1847 of Table 3C. In some embodiments, the prime editing composition further comprises a ngRNA that comprises a spacer comprising a sequence selected from the group consisting of SEQ Identifiers: D385-D386 of Table 3D.

In some embodiments, the PEgRNA comprises a spacer comprising a sequence selected from the group consisting of SEQ Identifiers: A139-A142 of Table 3A. In some embodiments, the PEgRNA comprises a PBS comprising a sequence selected from the group consisting of SEQ Identifiers: B594-B602 of Table 3B. In some embodiments, the PEgRNA comprises an editing template comprising a sequence selected from the group consisting of SEQ Identifiers: C1848-C1874 of Table 3C. In some embodiments, the prime editing composition further comprises a ngRNA that comprises a spacer comprising a sequence selected from the group consisting of SEQ Identifiers: D326-D330, D343-d347, D349, D350, D354, D359, D360, D434-D450, and 452-D459 of Table 3D.

In some embodiments, the PEgRNA comprises a spacer comprising a sequence selected from the group consisting of SEQ Identifiers: A143-A144 of Table 3A. In some embodiments, the PEgRNA comprises a PBS comprising a sequence selected from the group consisting of SEQ Identifiers: B603-B611 of Table 3B. In some embodiments, the PEgRNA comprises an editing template comprising a sequence selected from the group consisting of SEQ Identifiers: C1875-C1880 of Table 3C. In some embodiments, the PEgRNA comprises a spacer comprising the sequence of SEQ Identifier: A145 of Table 3A. In some embodiments, the PEgRNA comprises a PBS comprising a sequence selected from the group consisting of SEQ Identifiers: B612-B620 of Table 3B. In some embodiments, the PEgRNA comprises an editing template comprising a sequence selected from the group consisting of SEQ Identifiers: C1881-C1884 of Table 3C. In some embodiments, the prime editing composition further comprises a ngRNA that comprises a spacer comprising a sequence selected from the group consisting of SEQ Identifiers: D363-D384 of Table 3D.

In some embodiments, the PEgRNA comprises a spacer comprising a sequence selected from the group consisting of SEQ Identifiers: A146-A147 of Table 3A. In some embodiments, the PEgRNA comprises a PBS comprising a sequence selected from the group consisting of SEQ Identifiers: B621-B629 of Table 3B. In some embodiments, the PEgRNA comprises an editing template comprising a sequence selected from the group consisting of SEQ Identifiers: C1885-C1906 of Table 3C. In some embodiments, the prime editing composition further comprises a ngRNA that comprises a spacer comprising a sequence selected from the group consisting of SEQ Identifiers: D332-D338, D349-D355, and D468-D470 of Table 3D.

In some embodiments, the PEgRNA comprises a spacer comprising the sequence of SEQ Identifier: A148 of Table 3A. In some embodiments, the PEgRNA comprises a PBS comprising a sequence selected from the group consisting of SEQ Identifiers: B630-B638 of Table 3B. In some embodiments, the PEgRNA comprises an editing template comprising a sequence selected from the group consisting of SEQ Identifiers: C1907-C1918 of Table 3C. In some embodiments, the prime editing composition further comprises a ngRNA that comprises a spacer comprising a sequence selected from the group consisting of SEQ Identifiers: D360, D430-D446, D453, and D454 of Table 3D.

In some embodiments, the PEgRNA comprises a spacer comprising a sequence selected from the group consisting of SEQ Identifiers: A149-A150 of Table 3A. In some embodiments, the PEgRNA comprises a PBS comprising a sequence selected from the group consisting of SEQ Identifiers: B639-B647 of Table 3B. In some embodiments, the PEgRNA comprises an editing template comprising a sequence selected from the group consisting of SEQ Identifiers: C1919-C1931 of Table 3C. In some embodiments, the prime editing composition further comprises a ngRNA that comprises a spacer comprising a sequence selected from the group consisting of SEQ Identifiers: D362-D384, D423, D424, D471, and D472 of Table 3D.

In some embodiments, the PEgRNA comprises a spacer comprising a sequence selected from the group consisting of SEQ Identifiers: A151-A152 of Table 3A. In some embodiments, the PEgRNA comprises a PBS comprising a sequence selected from the group consisting of SEQ Identifiers: B648-B656 of Table 3B. In some embodiments, the PEgRNA comprises an editing template comprising a sequence selected from the group consisting of SEQ Identifiers: C1932-C1952 of Table 3C. In some embodiments, the prime editing composition further comprises a ngRNA that comprises a spacer comprising a sequence selected from the group consisting of SEQ Identifiers: D362-D382, D423, D424, D427, and D471 of Table 3D.

In some embodiments, the PEgRNA comprises a spacer comprising a sequence selected from the group consisting of SEQ Identifiers: A153-A154 of Table 3A. In some embodiments, the PEgRNA comprises a PBS comprising a sequence selected from the group consisting of SEQ Identifiers: B657-B665 of Table 3B. In some embodiments, the PEgRNA comprises an editing template comprising a sequence selected from the group consisting of SEQ Identifiers: C1953-C1964 of Table 3C. In some embodiments, the prime editing composition further comprises a ngRNA that comprises a spacer comprising a sequence selected from the group consisting of SEQ Identifiers: D362-D384, and D397-D405 of Table 3D.

In some embodiments, the PEgRNA comprises a spacer comprising a sequence selected from the group consisting of SEQ Identifiers: A155-A157 of Table 3A. In some embodiments, the PEgRNA comprises a PBS comprising a sequence selected from the group consisting of SEQ Identifiers: B666-B674 of Table 3B. In some embodiments, the PEgRNA comprises an editing template comprising a sequence selected from the group consisting of SEQ Identifiers: C1965-C1995 of Table 3C. In some embodiments, the prime editing composition further comprises a ngRNA that comprises a spacer comprising a sequence selected from the group consisting of SEQ Identifiers: D328, D329, D330, D345, D346, D347, D350, D354, and D359 of Table 3D.

In some embodiments, the editing target sequence or the complementary sequence on the CFTR gene thereof comprises a mutation that encodes an amino acid substitution R334W, R334L, and/or I336K relative to a wild type CFTR polypeptide set forth in SEQ IDENTIFIER: 19, and wherein the editing template encodes an intended nucleotide edit that corrects the mutation. In some embodiments, the editing target sequence or the complementary sequence on the CFTR gene thereof comprises a C→→T mutation (c.1000C>T) at position 117,540,230, a G→→T mutation (c.1001G>T) at position 117,540,231, an insertion of G (c.1006_1007insG) at position 117,540,236, and/or a T→→A mutation (c.1007T>A) at position 117,540,237 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide. In some embodiments, the editing template comprises a wild type CFTR gene sequence. Accordingly, in some embodiments, the newly synthesized single stranded DNA encoded by the editing template is capable of correcting the c.1000C>T mutation, the c.1001G>T mutation, the c.1006_1007insG mutation, and/or the c.1007T>A mutation in the target CFTR gene. In some embodiments, the newly synthesized single stranded DNA encoded by the editing template is capable of correcting all of the c.1000C>T mutation, the c.1001G>T mutation, the c.1006_1007insG mutation, and the c.1007T>A mutation in the target CFTR gene. In some embodiments, two or more of the c.1000C>T mutation, the c.1001G>T mutation, the c.1006_1007insG mutation, and the c.1007T>A mutation are located on the same CFTR gene. In some embodiments, the newly synthesized single stranded DNA encoded by the editing template is capable of correcting the c.1000C>T mutation, the c.1001G>T mutation, the c.1006_1007insG mutation, and the c.1007T>A mutation that are located on different CFTR genes. For example, in some embodiments, a subject may comprise two copies of CFTR genes, each comprising a different mutation selected from the group consisting of the c.1000C>T mutation, the c.1001G>T mutation, the c.1006_1007insG mutation, and the c.1007T>A mutation, all of which may be corrected by an editing template of a single PEgRNA. In some embodiments, the editing template is capable of correcting one or more, or all of the c.1000C>T mutation, the c.1001G>T mutation, the c.1006_1007insG mutation, and the c.1007T>A mutation that are in different subjects, wherein each subject may have one or more of the mutations.

In some embodiments, the PEgRNA comprises a spacer comprising a sequence selected from the group consisting of SEQ Identifiers: A158-A159 of Table 3A. In some embodiments, the PEgRNA comprises a PBS comprising a sequence selected from the group consisting of SEQ Identifiers: B675-B683 of Table 3B. In some embodiments, the PEgRNA comprises an editing template comprising a sequence selected from the group consisting of SEQ Identifiers: C1996-C2057 of Table 3.

In some embodiments, the prime editing composition further comprises a ngRNA that comprises a spacer comprising a sequence selected from the group consisting of SEQ Identifiers: D439-D446, D450, D452-D457, and D473-D488 of Table 3D. In some embodiments, the PEgRNA comprises a spacer comprising a sequence selected from the group consisting of SEQ Identifiers: A160-A161 of Table 3A. In some embodiments, the PEgRNA comprises a PBS comprising a sequence selected from the group consisting of SEQ Identifiers: B684-B692 of Table 3B. In some embodiments, the PEgRNA comprises an editing template comprising a sequence selected from the group consisting of SEQ Identifiers: C2058-C2099 of Table 3C. In some embodiments, the prime editing composition further comprises a ngRNA that comprises a spacer comprising a sequence selected from the group consisting of SEQ Identifiers: D366-D384, D367-D384, D398-D404, and D489-D495 of Table 3D. In some embodiments, the PEgRNA comprises a spacer comprising the sequence of SEQ Identifiers: A162 of Table 3A. In some embodiments, the PEgRNA comprises a PBS comprising a sequence selected from the group consisting of SEQ Identifiers: B693-B701 of Table 3B. In some embodiments, the PEgRNA comprises an editing template comprising a sequence selected from the group consisting of SEQ Identifiers: C2100-C2121 of Table 3C. In some embodiments, the prime editing composition further comprises a ngRNA that comprises a spacer comprising a sequence selected from the group consisting of SEQ Identifiers: D366-D384, and D 489-D494 of Table 3D.

In some embodiments, the PEgRNA comprises a spacer comprising a sequence selected from the group consisting of SEQ Identifiers: A163-A164 of Table 3A. In some embodiments, the PEgRNA comprises a PBS comprising a sequence selected from the group consisting of SEQ Identifiers: B702-B710 of Table 3B. In some embodiments, the PEgRNA comprises an editing template comprising a sequence selected from the group consisting of SEQ Identifiers: C2122-C2157 of Table 3C. In some embodiments, the prime editing composition further comprises a ngRNA that comprises a spacer comprising a sequence selected from the group consisting of SEQ Identifiers: D366-D384, D398-D404, and D489-D495 of Table 3D.

In some embodiments, the PEgRNA comprises a spacer comprising the sequence of SEQ Identifier: A165 of Table 3A. In some embodiments, the PEgRNA comprises a PBS comprising a sequence selected from the group consisting of SEQ Identifiers: B711-B719 of Table 3B. In some embodiments, the PEgRNA comprises an editing template comprising a sequence selected from the group consisting of SEQ Identifiers: C2158-C2176 of Table 3C. In some embodiments, the prime editing composition further comprises a ngRNA that comprises a spacer comprising a sequence selected from the group consisting of SEQ Identifiers: D366-DD384, and D489-D494 of Table 3D.

In some embodiments, the PEgRNA comprises a spacer comprising a sequence selected from the group consisting of SEQ Identifiers: A166-A167 of Table 3A. In some embodiments, the PEgRNA comprises a PBS comprising a sequence selected from the group consisting of SEQ Identifiers: B720-B728 of Table 3B. In some embodiments, the PEgRNA comprises an editing template comprising a sequence selected from the group consisting of SEQ Identifiers: C2177-C2207 of Table 3C. In some embodiments, the prime editing composition further comprises a ngRNA that comprises a spacer comprising a sequence selected from the group consisting of SEQ Identifiers: D440-D446, D453-D457, D475-D488, and D496-D506 of Table 3D.

In some embodiments, the PEgRNA comprises a spacer comprising a sequence selected from the group consisting of SEQ Identifiers: A168-A169 of Table 3A. In some embodiments, the PEgRNA comprises a PBS comprising a sequence selected from the group consisting of SEQ Identifiers: B729-B737 of Table 3B. In some embodiments, the PEgRNA comprises an editing template comprising a sequence selected from the group consisting of SEQ Identifiers: C2208-C2231 of Table 3C. In some embodiments, the prime editing composition further comprises a ngRNA that comprises a spacer comprising a sequence selected from the group consisting of SEQ Identifiers: D366-d384, D471, D472, and D489-D494 of Table 3D.

In some embodiments, the PEgRNA comprises a spacer comprising a sequence selected from the group consisting of SEQ Identifiers: A170-A171 of Table 3A. In some embodiments, the PEgRNA comprises a PBS comprising a sequence selected from the group consisting of SEQ Identifiers: B738-B746 of Table 3B. In some embodiments, the PEgRNA comprises an editing template comprising a sequence selected from the group consisting of SEQ Identifiers: C2232-C2262 of Table 3C. In some embodiments, the prime editing composition further comprises a ngRNA that comprises a spacer comprising a sequence selected from the group consisting of SEQ Identifiers: D366-D384, D471, D472, and D489-D492 of Table 3D.

In some embodiments, the PEgRNA comprises a spacer comprising a sequence selected from the group consisting of SEQ Identifiers: A172-A174 of Table 3A. In some embodiments, the PEgRNA comprises a PBS comprising a sequence selected from the group consisting of SEQ Identifiers: B747-B755 of Table 3B. In some embodiments, the PEgRNA comprises an editing template comprising a sequence selected from the group consisting of SEQ Identifiers: C2263-C2285 of Table 3C. In some embodiments, the PEgRNA comprises a spacer comprising a sequence selected from the group consisting of SEQ Identifiers: A175-A176 of Table 3A. In some embodiments, the PEgRNA comprises a PBS comprising a sequence selected from the group consisting of SEQ Identifiers: B756-B764 of Table 3B. In some embodiments, the PEgRNA comprises an editing template comprising a sequence selected from the group consisting of SEQ Identifiers: C2286-C2300 of Table 3C. In some embodiments, the prime editing composition further comprises a ngRNA that comprises a spacer comprising a sequence selected from the group consisting of SEQ Identifiers: D367-D384, D471, D472, D489-D494, D507, and D508 of Table 3D.

In some embodiments, the PEgRNA comprises a spacer comprising a sequence selected from the group consisting of SEQ Identifiers: A177-A180 of Table 3A. In some embodiments, the PEgRNA comprises a PBS comprising a sequence selected from the group consisting of SEQ Identifiers: B765-B773 of Table 3B. In some embodiments, the PEgRNA comprises an editing template comprising a sequence selected from the group consisting of SEQ Identifiers: C2301-C2316 of Table 3C. In some embodiments, the prime editing composition further comprises a ngRNA that comprises a spacer comprising a sequence selected from the group consisting of SEQ Identifiers: D509-D512 of Table 3D.

In some embodiments, the PEgRNA comprises a spacer comprising a sequence selected from the group consisting of SEQ Identifiers: A181-A183 of Table 3A. In some embodiments, the PEgRNA comprises a PBS comprising a sequence selected from the group consisting of SEQ Identifiers: B774-B782 of Table 3B. In some embodiments, the PEgRNA comprises an editing template comprising a sequence selected from the group consisting of SEQ Identifiers: C2317-C2347 of Table 3C. In some embodiments, the prime editing composition further comprises a ngRNA that comprises a spacer comprising a sequence selected from the group consisting of SEQ Identifiers: D329, D346, D354, D485, and D513-D 520 of Table 3D.

In some embodiments, the PEgRNA comprises a spacer comprising a sequence selected from the group consisting of SEQ Identifiers: A184-A187 of Table 3A. In some embodiments, the PEgRNA comprises a PBS comprising a sequence selected from the group consisting of SEQ Identifiers: B783-B791 of Table 3B. In some embodiments, the PEgRNA comprises an editing template comprising a sequence selected from the group consisting of SEQ Identifiers: C2348-C2391 of Table 3C. In some embodiments, the prime editing composition further comprises a ngRNA that comprises a spacer comprising a sequence selected from the group consisting of SEQ Identifiers: D329, D334-D338, D341, D346, D351-D355, D358, D485, and D513-D538 of Table 3D.

In some embodiments, the PEgRNA comprises a spacer comprising the sequence of SEQ Identifiers: A188 of Table 3A. In some embodiments, the PEgRNA comprises a PBS comprising a sequence selected from the group consisting of SEQ Identifiers: B792-B800 of Table 3B. In some embodiments, the PEgRNA comprises an editing template comprising the sequence of SEQ Identifiers: c2392 of Table 3C. In some embodiments, the prime editing composition further comprises a ngRNA that comprises a spacer comprising a sequence selected from the group consisting of SEQ Identifiers: D439-D446, D453-D457, and D475-D480 of Table 3D.

In some embodiments, the PEgRNA comprises a spacer comprising a sequence selected from the group consisting of SEQ Identifiers: A189-A191 of Table 3A. In some embodiments, the PEgRNA comprises a PBS comprising a sequence selected from the group consisting of SEQ Identifiers: B801-B809 of Table 3B. In some embodiments, the PEgRNA comprises an editing template comprising a sequence selected from the group consisting of SEQ Identifiers: C2393-C2412 of Table 3C. In some embodiments, the prime editing composition further comprises a ngRNA that comprises a spacer comprising a sequence selected from the group consisting of SEQ Identifiers: D376, D389-D395, D407-D412, D416-D422, D510, D539, and D540 of Table 3D.

In some embodiments, the PEgRNA comprises a spacer comprising a sequence selected from the group consisting of SEQ Identifiers: A192 of Table 3A. In some embodiments, the PEgRNA comprises a PBS comprising a sequence selected from the group consisting of SEQ Identifiers: B810-B818 of Table 3B. In some embodiments, the PEgRNA comprises an editing template comprising a sequence selected from the group consisting of SEQ Identifiers: C2413-C2418 of Table 3C. In some embodiments, the prime editing composition further comprises a ngRNA that comprises a spacer comprising a sequence selected from the group consisting of SEQ Identifiers: D369-D384, D489-D494, and D508 of Table 3D.

In some embodiments, the PEgRNA comprises a spacer comprising a sequence selected from the group consisting of SEQ Identifiers: A193-A194 of Table 3A. In some embodiments, the PEgRNA comprises a PBS comprising a sequence selected from the group consisting of SEQ Identifiers: B819-B827 of Table 3B. In some embodiments, the PEgRNA comprises an editing template comprising a sequence selected from the group consisting of SEQ Identifiers: C2419-C2449 of Table 3C. In some embodiments, the prime editing composition further comprises a ngRNA that comprises a spacer comprising a sequence selected from the group consisting of SEQ Identifiers: D509-D512 of Table 3D.

In some embodiments, the PEgRNA comprises a spacer comprising a sequence selected from the group consisting of SEQ Identifiers: A195-A197 of Table 3A. In some embodiments, the PEgRNA comprises a PBS comprising a sequence selected from the group consisting of SEQ Identifiers: B828-B836 of Table 3B. In some embodiments, the PEgRNA comprises an editing template comprising a sequence selected from the group consisting of SEQ Identifiers: C2450-C2466 of Table 3C. In some embodiments, the prime editing composition further comprises a ngRNA that comprises a spacer comprising a sequence selected from the group consisting of SEQ Identifiers: D376, D389-D395, D407-D412, D416-D422, D510, D539, and D540 of Table 3D.

In some embodiments, the PEgRNA comprises a spacer comprising a sequence selected from the group consisting of SEQ Identifiers: A198-A199 of Table 3A. In some embodiments, the PEgRNA comprises a PBS comprising a sequence selected from the group consisting of SEQ Identifiers: B837-B845 of Table 3B. In some embodiments, the PEgRNA comprises an editing template comprising a sequence selected from the group consisting of SEQ Identifiers: C2467-C2475 of Table 3C. In some embodiments, the prime editing composition further comprises a ngRNA that comprises a spacer comprising a sequence selected from the group consisting of SEQ Identifiers: D368-DD384, D471, D472, D489-D494, D507, and D508 of Table 3D.

In some embodiments, the PEgRNA comprises a spacer comprising a sequence selected from the group consisting of SEQ Identifiers: A200-A201 of Table 3A. In some embodiments, the PEgRNA comprises a PBS comprising a sequence selected from the group consisting of SEQ Identifiers: B846-B854 of Table 3B. In some embodiments, the PEgRNA comprises an editing template comprising a sequence selected from the group consisting of SEQ Identifiers: C2476-C2503 of Table 3C. In some embodiments, the prime editing composition further comprises a ngRNA that comprises a spacer comprising a sequence selected from the group consisting of SEQ Identifiers: D509-D512 of Table 3D.

In some embodiments, the PEgRNA comprises a spacer comprising a sequence selected from the group consisting of SEQ Identifiers: A202-A203 of Table 3A. In some embodiments, the PEgRNA comprises a PBS comprising a sequence selected from the group consisting of SEQ Identifiers: B855-B863 of Table 3B. In some embodiments, the PEgRNA comprises an editing template comprising a sequence selected from the group consisting of SEQ Identifiers: C2504-C2565 of Table 3C. In some embodiments, the prime editing composition further comprises a ngRNA that comprises a spacer comprising a sequence selected from the group consisting of SEQ Identifiers: D366-D384, D398-D404, D489D492, and D495 of Table 3D.

In some embodiments, the PEgRNA comprises a spacer comprising a sequence selected from the group consisting of SEQ Identifiers: A204-A205 of Table 3A. In some embodiments, the PEgRNA comprises a PBS comprising a sequence selected from the group consisting of SEQ Identifiers: B864-B872 of Table 3B. In some embodiments, the PEgRNA comprises an editing template comprising a sequence selected from the group consisting of SEQ Identifiers: C2566-C2596 of Table 3C. In some embodiments, the prime editing composition further comprises a ngRNA that comprises a spacer comprising a sequence selected from the group consisting of SEQ Identifiers: D366-D384, D471, D472, and D489-D492 of Table 3D.

In some embodiments, the PEgRNA comprises a spacer comprising a sequence selected from the group consisting of SEQ Identifiers: A206-A207 of Table 3A. In some embodiments, the PEgRNA comprises a PBS comprising a sequence selected from the group consisting of SEQ Identifiers: B873-B881 of Table 3B. In some embodiments, the PEgRNA comprises an editing template comprising a sequence selected from the group consisting of SEQ Identifiers: C2597-C2620 of Table 3C. In some embodiments, the prime editing composition further comprises a ngRNA that comprises a spacer comprising a sequence selected from the group consisting of SEQ Identifiers: D439-D446, D453-D457, D475-D487, and D496D505 of Table 3D.

In some embodiments, the PEgRNA comprises a spacer comprising a sequence selected from the group consisting of SEQ Identifiers: A208-A209 of Table 3A. In some embodiments, the PEgRNA comprises a PBS comprising a sequence selected from the group consisting of SEQ Identifiers: B882-B890 of Table 3B. In some embodiments, the PEgRNA comprises an editing template comprising a sequence selected from the group consisting of SEQ Identifiers: C2621-C2633 of Table 3C. In some embodiments, the prime editing composition further comprises a ngRNA that comprises a spacer comprising a sequence selected from the group consisting of SEQ Identifiers: D367-D384, D471, D472, D489-D494, D507, and D508 of Table 3D.

In some embodiments, the PEgRNA comprises a spacer comprising a sequence selected from the group consisting of SEQ Identifiers: A210-A213 of Table 3A. In some embodiments, the PEgRNA comprises a PBS comprising a sequence selected from the group consisting of SEQ Identifiers: B891-B899 of Table 3B. In some embodiments, the PEgRNA comprises an editing template comprising a sequence selected from the group consisting of SEQ Identifiers: C2634-C2695 of Table 3C. In some embodiments, the prime editing composition further comprises a ngRNA that comprises a spacer comprising a sequence selected from the group consisting of SEQ Identifiers: D376, D389-D395, D407-D412, D416-D422, D509-D512, D539, and D540 of Table 3D.

In some embodiments, the PEgRNA comprises a spacer comprising a sequence selected from the group consisting of SEQ Identifiers: A214-A217 of Table 3A. In some embodiments, the PEgRNA comprises a PBS comprising a sequence selected from the group consisting of SEQ Identifiers: B900-B908 of Table 3B. In some embodiments, the PEgRNA comprises an editing template comprising a sequence selected from the group consisting of SEQ Identifiers: C2696-C2718 of Table 3C. In some embodiments, the prime editing composition further comprises a ngRNA that comprises a spacer comprising a sequence selected from the group consisting of SEQ Identifiers: D331, D348, D361, D439-D446, D450, D452-D457, and D473-D486 of Table 3D.

In some embodiments, the PEgRNA comprises a spacer comprising the sequence of SEQ Identifier: A218 of Table 3A. In some embodiments, the PEgRNA comprises a PBS comprising a sequence selected from the group consisting of SEQ Identifiers: B909-B917 of Table 3B. In some embodiments, the PEgRNA comprises an editing template comprising a sequence selected from the group consisting of SEQ Identifiers: C2719-C2729 of Table 3C. In some embodiments, the prime editing composition further comprises a ngRNA that comprises a spacer comprising a sequence selected from the group consisting of SEQ Identifiers: D439-D457, and D475-D481 of Table 3D.

In some embodiments, the editing target sequence or the complementary sequence on the CFTR gene thereof comprises a mutation that encodes an amino acid substitution R347H or R347P relative to a wild type CFTR polypeptide set forth in SEQ IDENTIFIER: 19, and wherein the editing template encodes an intended nucleotide edit that corrects the mutation. In some embodiments, the editing target sequence or the complementary sequence on the CFTR gene thereof comprises a G→→A mutation (c.1040G>A) at position 117,540,270, a G→A mutation (c.1040G>C) at position 117,540,270, a G→→T mutation (c.1040G>T) at position 117,540,270, and/or a T→→A mutation (c.1043T>A) at position 117,540,273 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide. In some embodiments, the editing template comprises a wild type CFTR gene sequence. Accordingly, in some embodiments, the newly synthesized single stranded DNA encoded by the editing template is capable of correcting the c.1040G>A mutation, the c.1040G>C mutation, the c.1040G>T mutation, and/or the c.1043T>A mutation in the target CFTR gene. In some embodiments, the newly synthesized single stranded DNA encoded by the editing template is capable of correcting all of the c.1040G>A mutation, the c.1040G>C mutation, the c.1040G>T mutation, and the c.1043T>A mutation in the target CFTR gene. In some embodiments, two or more of the c.1040G>A mutation, the c.1040G>C mutation, the c.1040G>T mutation, and the c.1043T>A mutation are located on the same CFTR gene. In some embodiments, the newly synthesized single stranded DNA encoded by the editing template is capable of correcting the c.1040G>A mutation, the c.1040G>C mutation, the c.1040G>T mutation, and the c.1043T>A mutation that are located on different CFTR genes. For example, in some embodiments, a subject may comprise two copies of CFTR genes, each comprising a different mutation selected from the group consisting of the c.1040G>A mutation, the c.1040G>C mutation, the c.1040G>T mutation, and the c.1043T>A mutation, all of which may be corrected by an editing template of a single PEgRNA. In some embodiments, the editing template is capable of correcting one or more, or all of the c.1040G>A mutation, the c.1040G>C mutation, the c.1040G>T mutation, and the c.1043T>A mutation that are in different subjects, wherein each subject may have one or more of the mutations.

In some embodiments, the PEgRNA comprises a spacer comprising a sequence selected from the group consisting of SEQ Identifiers: A219-A222 of Table 3A. In some embodiments, the PEgRNA comprises a PBS comprising a sequence selected from the group consisting of SEQ Identifiers: B918-B926 of Table 3B. In some embodiments, the PEgRNA comprises an editing template comprising a sequence selected from the group consisting of SEQ Identifiers: C2730-C2760 of Table 3C. In some embodiments, the prime editing composition further comprises a ngRNA that comprises a spacer comprising a sequence selected from the group consisting of SEQ Identifiers: D376-D384, D392-D395, D401D404, D410-D412, D419-D422, D489-D495, D508, D510, and D539-D 548 of Table 3D.

In some embodiments, the PEgRNA comprises a spacer comprising a sequence selected from the group consisting of SEQ Identifiers: A223-A224 of Table 3A. In some embodiments, the PEgRNA comprises a PBS comprising a sequence selected from the group consisting of SEQ Identifiers: B927-B935 of Table 3B. In some embodiments, the PEgRNA comprises an editing template comprising a sequence selected from the group consisting of SEQ Identifiers: C2761-C2804 of Table 3C. In some embodiments, the prime editing composition further comprises a ngRNA that comprises a spacer comprising a sequence selected from the group consisting of SEQ Identifiers: D509-D512, D549-D554 of Table 3D.

In some embodiments, the PEgRNA comprises a spacer comprising the sequence of SEQ Identifier: A225 of Table 3A. In some embodiments, the PEgRNA comprises a PBS comprising a sequence selected from the group consisting of SEQ Identifiers: B936-B944 of Table 3B. In some embodiments, the PEgRNA comprises an editing template comprising a sequence selected from the group consisting of SEQ Identifiers: C2805-C2827 of Table 3C. In some embodiments, the prime editing composition further comprises a ngRNA that comprises a spacer comprising a sequence selected from the group consisting of SEQ Identifiers: D376-D384, D489-D494, D508, D544, D545, and D555 of Table 3D.

In some embodiments, the PEgRNA comprises a spacer comprising the sequence of SEQ Identifiers: A226 of Table 3A. In some embodiments, the PEgRNA comprises a PBS comprising a sequence selected from the group consisting of SEQ Identifiers: B945-B953 of Table 3B. In some embodiments, the PEgRNA comprises an editing template comprising a sequence selected from the group consisting of SEQ Identifiers: C2828-C2855 of Table 3.

In some embodiments, the prime editing composition further comprises a ngRNA that comprises a spacer comprising a sequence selected from the group consisting of SEQ Identifiers: D453-D457, D475-D486, and D556-D569 of Table 3D.

In some embodiments, the PEgRNA comprises a spacer comprising a sequence selected from the group consisting of SEQ Identifiers: A227-A228 of Table 3A. In some embodiments, the PEgRNA comprises a PBS comprising a sequence selected from the group consisting of SEQ Identifiers: B954-B962 of Table 3B. In some embodiments, the PEgRNA comprises an editing template comprising a sequence selected from the group consisting of SEQ Identifiers: C2856-C2866 of Table 3C. In some embodiments, the prime editing composition further comprises a ngRNA that comprises a spacer comprising a sequence selected from the group consisting of SEQ Identifiers: D380-D384, D403, D404, D489-D 495, D508, D544, D545, D555, D570, and D571 of Table 3D.

In some embodiments, the PEgRNA comprises a spacer comprising a sequence selected from the group consisting of SEQ Identifiers: A229-A232 of Table 3A. In some embodiments, the PEgRNA comprises a PBS comprising a sequence selected from the group consisting of SEQ Identifiers: B963-B971 of Table 3B. In some embodiments, the PEgRNA comprises an editing template comprising a sequence selected from the group consisting of SEQ Identifiers: C2867-C2884 of Table 3C. In some embodiments, the prime editing composition further comprises a ngRNA that comprises a spacer comprising a sequence selected from the group consisting of SEQ Identifiers: D394, D395, D411, D412, D421, D422, D509-D512, and D539, D540, D549-D554 of Table 3D.

In some embodiments, the PEgRNA comprises a spacer comprising a sequence selected from the group consisting of SEQ Identifiers: A233-A234 of Table 3A. In some embodiments, the PEgRNA comprises a PBS comprising a sequence selected from the group consisting of SEQ Identifiers: B972-B980 of Table 3B. In some embodiments, the PEgRNA comprises an editing template comprising a sequence selected from the group consisting of SEQ Identifiers: C2885-C2915 of Table 3C. In some embodiments, the prime editing composition further comprises a ngRNA that comprises a spacer comprising a sequence selected from the group consisting of SEQ Identifiers: D376-D384, D401-D404, D489-D495, D508, D542, D543, D544, and D546 of Table 3D.

In some embodiments, the PEgRNA comprises a spacer comprising the sequence of SEQ Identifier: A235 of Table 3A. In some embodiments, the PEgRNA comprises a PBS comprising a sequence selected from the group consisting of SEQ Identifiers: B981-B989 of Table 3B. In some embodiments, the PEgRNA comprises an editing template comprising a sequence selected from the group consisting of SEQ Identifiers: C2916-C2946 of Table 3C. In some embodiments, the prime editing composition further comprises a ngRNA that comprises a spacer comprising a sequence selected from the group consisting of SEQ Identifiers: D376-D384, D489-d494, D508, D542, D543, and D544 of Table 3D.

In some embodiments, the PEgRNA comprises a spacer comprising the sequence of SEQ Identifier: A236 of Table 3A. In some embodiments, the PEgRNA comprises a PBS comprising a sequence selected from the group consisting of SEQ Identifiers: B990-B998 of Table 3B. In some embodiments, the PEgRNA comprises an editing template comprising a sequence selected from the group consisting of SEQ Identifiers: C2947-C2975 of Table 3C. In some embodiments, the prime editing composition further comprises a ngRNA that comprises a spacer comprising a sequence selected from the group consisting of SEQ Identifiers: D376-D384, D489-D494, D508, D543, D544, D545, and D555 of Table 3D.

In some embodiments, the PEgRNA comprises a spacer comprising the sequence of SEQ Identifiers: A237 of Table 3A. In some embodiments, the PEgRNA comprises a PBS comprising a sequence selected from the group consisting of SEQ Identifiers: B999-B1007 of Table 3B. In some embodiments, the PEgRNA comprises an editing template comprising a sequence selected from the group consisting of SEQ Identifiers: C2976-C3006 of Table 3C. In some embodiments, the prime editing composition further comprises a ngRNA that comprises a spacer comprising a sequence selected from the group consisting of SEQ Identifiers: D453-D457, D475-D486, D556-D569, and D572 of Table 3D.

In some embodiments, the PEgRNA comprises a spacer comprising a sequence selected from the group consisting of SEQ Identifiers: A238-A240 of Table 3A. In some embodiments, the PEgRNA comprises a PBS comprising a sequence selected from the group consisting of SEQ Identifiers: B1008-B1016 of Table 3B. In some embodiments, the PEgRNA comprises an editing template comprising a sequence selected from the group consisting of SEQ Identifiers: C3007-C3037 of Table 3C. In some embodiments, the prime editing composition further comprises a ngRNA that comprises a spacer comprising a sequence selected from the group consisting of SEQ Identifiers: D485, D513-D520, and D573-D584 of Table 3D.

In some embodiments, the PEgRNA comprises a spacer comprising a sequence selected from the group consisting of SEQ Identifiers: A241-A242 of Table 3A. In some embodiments, the PEgRNA comprises a PBS comprising a sequence selected from the group consisting of SEQ Identifiers: B1017-B1025 of Table 3B. In some embodiments, the PEgRNA comprises an editing template comprising a sequence selected from the group consisting of SEQ Identifiers: C3038-C3049 of Table 3C. In some embodiments, the prime editing composition further comprises a ngRNA that comprises a spacer comprising a sequence selected from the group consisting of SEQ Identifiers: D380-D384, D471D472, D489-D494, D507, D508, D544, D545, D555, D570, D571, D585, and D586 of Table 3D.

In some embodiments, the PEgRNA comprises a spacer comprising a sequence selected from the group consisting of SEQ Identifiers: A243-A244 of Table 3A. In some embodiments, the PEgRNA comprises a PBS comprising a sequence selected from the group consisting of SEQ Identifiers: B1026-B1034 of Table 3B. In some embodiments, the PEgRNA comprises an editing template comprising a sequence selected from the group consisting of SEQ Identifiers: C3050-C3080 of Table 3C. In some embodiments, the prime editing composition further comprises a ngRNA that comprises a spacer comprising a sequence selected from the group consisting of SEQ Identifiers: D376-D384, D471D472, D489-D494, D507, D508, D542, D543, D544, D587, and D588 of Table 3D.

In some embodiments, the PEgRNA comprises a spacer comprising a sequence selected from the group consisting of SEQ Identifiers: A245-A247 of Table 3A. In some embodiments, the PEgRNA comprises a PBS comprising a sequence selected from the group consisting of SEQ Identifiers: B1035-B1043 of Table 3B. In some embodiments, the PEgRNA comprises an editing template comprising a sequence selected from the group consisting of SEQ Identifiers: C3081-C3111 of Table 3C. In some embodiments, the prime editing composition further comprises a ngRNA that comprises a spacer comprising a sequence selected from the group consisting of SEQ Identifiers: D376, D392-D395, D410D412, D419, D422, D510, D539, D540, D541, D547, and D548 of Table 3D.

In some embodiments, the PEgRNA comprises a spacer comprising a sequence selected from the group consisting of SEQ Identifiers: A248-A249 of Table 3A. In some embodiments, the PEgRNA comprises a PBS comprising a sequence selected from the group consisting of SEQ Identifiers: B1044-B1052 of Table 3B. In some embodiments, the PEgRNA comprises an editing template comprising a sequence selected from the group consisting of SEQ Identifiers: C3112-C3119 of Table 3C. In some embodiments, the prime editing composition further comprises a ngRNA that comprises a spacer comprising a sequence selected from the group consisting of SEQ Identifiers: D509D512, and D549-D554 of Table 3D.

In some embodiments, the PEgRNA comprises a spacer comprising the sequence of SEQ Identifier: A250 of Table 3A. In some embodiments, the PEgRNA comprises a PBS comprising a sequence selected from the group consisting of SEQ Identifiers: B1053-B1061 of Table 3B. In some embodiments, the PEgRNA comprises an editing template comprising a sequence selected from the group consisting of SEQ Identifiers: C3120-C3124 of Table 3C. In some embodiments, the prime editing composition further comprises a ngRNA that comprises a spacer comprising a sequence selected from the group consisting of SEQ Identifiers: D380-D384, D489-D494, D508, D544, D545, D555, D570, and D571 of Table 3D.

In some embodiments, the PEgRNA comprises a spacer comprising a sequence selected from the group consisting of SEQ Identifiers: A251-A252 of Table 3A. In some embodiments, the PEgRNA comprises a PBS comprising a sequence selected from the group consisting of SEQ Identifiers: B1062-B1070 of Table 3B. In some embodiments, the PEgRNA comprises an editing template comprising a sequence selected from the group consisting of SEQ Identifiers: C3125-C3149 of Table 3C. In some embodiments, the prime editing composition further comprises a ngRNA that comprises a spacer comprising a sequence selected from the group consisting of SEQ Identifiers: D376-D384, D471, D472, D489-D494, D507, D508, D544, D545, D555, and D585 of Table 3D.

In some embodiments, the PEgRNA comprises a spacer comprising a sequence selected from the group consisting of SEQ Identifiers: A253-A254 of Table 3A. In some embodiments, the PEgRNA comprises a PBS comprising a sequence selected from the group consisting of SEQ Identifiers: B1071-B1079 of Table 3B. In some embodiments, the PEgRNA comprises an editing template comprising a sequence selected from the group consisting of SEQ Identifiers: C3150-C3211 of Table 3C. In some embodiments, the prime editing composition further comprises a ngRNA that comprises a spacer comprising a sequence selected from the group consisting of SEQ Identifiers: D509-D512, D549, and D552 of Table 3D.

In some embodiments, the PEgRNA comprises a spacer comprising a sequence selected from the group consisting of SEQ Identifiers: A255-A256 of Table 3A. In some embodiments, the PEgRNA comprises a PBS comprising a sequence selected from the group consisting of SEQ Identifiers: B1080-B1088 of Table 3B. In some embodiments, the PEgRNA comprises an editing template comprising a sequence selected from the group consisting of SEQ Identifiers: C3212-C3232 of Table 3C. In some embodiments, the prime editing composition further comprises a ngRNA that comprises a spacer comprising a sequence selected from the group consisting of SEQ Identifiers: D376-D384, D471, D472, D489-D494, D507, D508, D544, D545, D555, D570, D585, and D586 of Table 3.

In some embodiments, the PEgRNA comprises a spacer comprising a sequence selected from the group consisting of SEQ Identifiers: A257-A260 of Table 3A. In some embodiments, the PEgRNA comprises a PBS comprising a sequence selected from the group consisting of SEQ Identifiers: B1089-B1097 of Table 3B. In some embodiments, the PEgRNA comprises an editing template comprising a sequence selected from the group consisting of SEQ Identifiers: C3233-C3252 of Table 3C. In some embodiments, the prime editing composition further comprises a ngRNA that comprises a spacer comprising a sequence selected from the group consisting of SEQ Identifiers: D380-D384, D394, D395, D403, D404, D411, D412, D421, D422, D489-D495, D508, D510, D539, D540, D544, D545, D555, D570, D571, D589, and D590 of Table 3D.

In some embodiments, the PEgRNA comprises a spacer comprising the sequence of SEQ Identifier: A261 of Table 3A. In some embodiments, the PEgRNA comprises a PBS comprising a sequence selected from the group consisting of SEQ Identifiers: B1098-B1106 of Table 3B. In some embodiments, the PEgRNA comprises an editing template comprising a sequence selected from the group consisting of SEQ Identifiers: C3253-C3275 of Table 3C. In some embodiments, the prime editing composition further comprises a ngRNA that comprises a spacer comprising a sequence selected from the group consisting of SEQ Identifiers: D445, D446, D453-D457, D475-D486, and D556-D 567 of Table 3D.

In some embodiments, the PEgRNA comprises a spacer comprising a sequence selected from the group consisting of SEQ Identifiers: A262-A264 of Table 3A. In some embodiments, the PEgRNA comprises a PBS comprising a sequence selected from the group consisting of SEQ Identifiers: B1107-B1115 of Table 3B. In some embodiments, the PEgRNA comprises an editing template comprising a sequence selected from the group consisting of SEQ Identifiers: C3276-C3295 of Table 3C. In some embodiments, the PEgRNA comprises a spacer comprising a sequence selected from the group consisting of SEQ Identifiers: A265-A266 of Table 3A. In some embodiments, the PEgRNA comprises a PBS comprising a sequence selected from the group consisting of SEQ Identifiers: B1116-B1124 of Table 3B. In some embodiments, the PEgRNA comprises an editing template comprising a sequence selected from the group consisting of SEQ Identifiers: C3296-C3326 of Table 3C. In some embodiments, the prime editing composition further comprises a ngRNA that comprises a spacer comprising a sequence selected from the group consisting of SEQ Identifiers: D452, D454-D457, D473-D486, D556-D569, D572, and D591-D 595 of Table 3D.

In some embodiments, the PEgRNA comprises a spacer comprising the sequence of SEQ Identifier: A267 of Table 3A. In some embodiments, the PEgRNA comprises a PBS comprising a sequence selected from the group consisting of SEQ Identifiers: B1125-B1133 of Table 3B. In some embodiments, the PEgRNA comprises an editing template comprising a sequence selected from the group consisting of SEQ Identifiers: C3327-C3344 of Table 3C. In some embodiments, the prime editing composition further comprises a ngRNA that comprises a spacer comprising a sequence selected from the group consisting of SEQ Identifiers: D376-D384, D489-D494, D508, D544, D545, D555, and D570 of Table 3D.

In some embodiments, the PEgRNA comprises a spacer comprising the sequence of SEQ Identifier: A220 of Table 3A. In some embodiments, the PEgRNA comprises a PBS comprising a sequence selected from the group consisting of SEQ Identifiers: B918-B926 of Table 3B. In some embodiments, the PEgRNA comprises an editing template comprising a sequence selected from the group consisting of SEQ Identifiers: C2730-C2760 of Table 3C. In some embodiments, the prime editing composition further comprises a ngRNA that comprises a spacer comprising a sequence selected from the group consisting of SEQ Identifiers: D376-D384, D489-d494, D508, D542, D543, D544, and D545 of Table 3D.

In some embodiments, the PEgRNA comprises a spacer comprising a sequence selected from the group consisting of SEQ Identifiers: A223-A224 of Table 3A. In some embodiments, the PEgRNA comprises a PBS comprising a sequence selected from the group consisting of SEQ Identifiers: B927-B935 of Table 3B. In some embodiments, the PEgRNA comprises an editing template comprising a sequence selected from the group consisting of SEQ Identifiers: C2761-C2804 of Table 3C. In some embodiments, the prime editing composition further comprises a ngRNA that comprises a spacer comprising a sequence selected from the group consisting of SEQ Identifiers: D509-D512, and D549-D 554 of Table 3D.

In some embodiments, the PEgRNA comprises a spacer comprising the sequence of SEQ Identifier: A225 of Table 3A. In some embodiments, the PEgRNA comprises a PBS comprising a sequence selected from the group consisting of SEQ Identifiers: B936-B944 of Table 3B. In some embodiments, the PEgRNA comprises an editing template comprising a sequence selected from the group consisting of SEQ Identifiers: C2805-C2827 of Table 3C. In some embodiments, the prime editing composition further comprises a ngRNA that comprises a spacer comprising a sequence selected from the group consisting of SEQ Identifiers: D376-D384, D489-D494, D508, D544, D545, and D555 of Table 3D.

In some embodiments, the PEgRNA comprises a spacer comprising the sequence of SEQ Identifier: A226 of Table 3A. In some embodiments, the PEgRNA comprises a PBS comprising a sequence selected from the group consisting of SEQ Identifiers: B945-B953 of Table 3B. In some embodiments, the PEgRNA comprises an editing template comprising a sequence selected from the group consisting of SEQ Identifiers: C2828-C2855 of Table 3C. In some embodiments, the prime editing composition further comprises a ngRNA that comprises a spacer comprising a sequence selected from the group consisting of SEQ Identifiers: D453-D457, D475-D486, and D556-D 569 of Table 3D.

In some embodiments, the PEgRNA comprises a spacer comprising a sequence selected from the group consisting of SEQ Identifiers: A227-A228 of Table 3A. In some embodiments, the PEgRNA comprises a PBS comprising a sequence selected from the group consisting of SEQ Identifiers: B954-B962 of Table 3B. In some embodiments, the PEgRNA comprises an editing template comprising a sequence selected from the group consisting of SEQ Identifiers: C2856-C2866 of Table 3C. In some embodiments, the prime editing composition further comprises a ngRNA that comprises a spacer comprising a sequence selected from the group consisting of SEQ Identifiers: D380-D384, D403, D404, D489-D495, D508, D544, D545, D555, D570, and D571 of Table 3D.

In some embodiments, the PEgRNA comprises a spacer comprising a sequence selected from the group consisting of SEQ Identifiers: A229-A232 of Table 3A. In some embodiments, the PEgRNA comprises a PBS comprising a sequence selected from the group consisting of SEQ Identifiers: B963-B971 of Table 3B. In some embodiments, the PEgRNA comprises an editing template comprising a sequence selected from the group consisting of SEQ Identifiers: C2867-C2884 of Table 3C. In some embodiments, the prime editing composition further comprises a ngRNA that comprises a spacer comprising a sequence selected from the group consisting of SEQ Identifiers: D394, D395, D411, D412, D421, D422, D509, D510, D511, D512, D539, D540, and D549-D 554 of Table 3D.

In some embodiments, the PEgRNA comprises a spacer comprising the sequence of SEQ Identifier: A235 of Table 3A. In some embodiments, the PEgRNA comprises a PBS comprising a sequence selected from the group consisting of SEQ Identifiers: B981-B989 of Table 3B. In some embodiments, the PEgRNA comprises an editing template comprising a sequence selected from the group consisting of SEQ Identifiers: C2916-C2946 of Table 3C. In some embodiments, the prime editing composition further comprises a ngRNA that comprises a spacer comprising a sequence selected from the group consisting of SEQ Identifiers: D376-D384, D489-D494, D508, D542, D543, and D544 of Table 3D.

In some embodiments, the PEgRNA comprises a spacer comprising the sequence of SEQ Identifier: A236 of Table 3A. In some embodiments, the PEgRNA comprises a PBS comprising a sequence selected from the group consisting of SEQ Identifiers: B990-B998 of Table 3B. In some embodiments, the PEgRNA comprises an editing template comprising a sequence selected from the group consisting of SEQ Identifiers: C2947-C2975 of Table 3C. In some embodiments, the prime editing composition further comprises a ngRNA that comprises a spacer comprising a sequence selected from the group consisting of SEQ Identifiers: D376-D384, D489-D494, D508, D543, D544, D545, and D555 of Table 3D.

In some embodiments, the PEgRNA comprises a spacer comprising the sequence of SEQ Identifier: A237 of Table 3A. In some embodiments, the PEgRNA comprises a PBS comprising a sequence selected from the group consisting of SEQ Identifiers: B999-B1007 of Table 3B. In some embodiments, the PEgRNA comprises an editing template comprising a sequence selected from the group consisting of SEQ Identifiers: C2976-C3006 of Table 3C. In some embodiments, the prime editing composition further comprises a ngRNA that comprises a spacer comprising a sequence selected from the group consisting of SEQ Identifiers: D453-D457, D475-D486, D556-D569, and D572 of Table 3D.

In some embodiments, the PEgRNA comprises a spacer comprising a sequence selected from the group consisting of SEQ Identifiers: A268-A270 of Table 3A. In some embodiments, the PEgRNA comprises a PBS comprising a sequence selected from the group consisting of SEQ Identifiers: B1008-B1016 of Table 3B. In some embodiments, the PEgRNA comprises an editing template comprising a sequence selected from the group consisting of SEQ Identifiers: C3007-C3037 of Table 3C. In some embodiments, the prime editing composition further comprises a ngRNA that comprises a spacer comprising a sequence selected from the group consisting of SEQ Identifiers: D485, D513-D520, and D573-D584 of Table 3D.

In some embodiments, the PEgRNA comprises a spacer comprising a sequence selected from the group consisting of SEQ Identifiers: A241-A242 of Table 3A. In some embodiments, the PEgRNA comprises a PBS comprising a sequence selected from the group consisting of SEQ Identifiers: B1017-B1025 of Table 3B. In some embodiments, the PEgRNA comprises an editing template comprising a sequence selected from the group consisting of SEQ Identifiers: C3038-C3049 of Table 3C. In some embodiments, the prime editing composition further comprises a ngRNA that comprises a spacer comprising a sequence selected from the group consisting of SEQ Identifiers: D380-D384, D471, D472, D489-D494, D507, D508, D544, D545, D555, D570, D571, D585, and D586 of Table 3D.

In some embodiments, the PEgRNA comprises a spacer comprising a sequence selected from the group consisting of SEQ Identifiers: A271-A272 of Table 3A. In some embodiments, the PEgRNA comprises a PBS comprising a sequence selected from the group consisting of SEQ Identifiers: B1026-B1034 of Table 3B. In some embodiments, the PEgRNA comprises an editing template comprising a sequence selected from the group consisting of SEQ Identifiers: C3050-C3080 of Table 3C. In some embodiments, the prime editing composition further comprises a ngRNA that comprises a spacer comprising a sequence selected from the group consisting of SEQ Identifiers: D376-D 384, D471, D472, D489-D494, D507, D508, D542, D543, D544, D587, and D588 of Table 3D.

In some embodiments, the PEgRNA comprises a spacer comprising a sequence selected from the group consisting of SEQ Identifiers: A248-A249 of Table 3A. In some embodiments, the PEgRNA comprises a PBS comprising a sequence selected from the group consisting of SEQ Identifiers: B1044-B1052 of Table 3B. In some embodiments, the PEgRNA comprises an editing template comprising a sequence selected from the group consisting of SEQ Identifiers: C3112-C3119 of Table 3C. In some embodiments, the prime editing composition further comprises a ngRNA that comprises a spacer comprising a sequence selected from the group consisting of SEQ Identifiers: D509-D512, and D549-D554 of Table 3D.

In some embodiments, the PEgRNA comprises a spacer comprising the of SEQ Identifiers: A250 of Table 3A. In some embodiments, the PEgRNA comprises a PBS comprising a sequence selected from the group consisting of SEQ Identifiers: B1053-B1061 of Table 3B. In some embodiments, the PEgRNA comprises an editing template comprising a sequence selected from the group consisting of SEQ Identifiers: C3120-C3124 of Table 3C. In some embodiments, the prime editing composition further comprises a ngRNA that comprises a spacer comprising a sequence selected from the group consisting of SEQ Identifiers: D380-D384, D489-D494, D508, D544, D545, D555, D570, and D571 of Table 3D.

In some embodiments, the PEgRNA comprises a spacer comprising a sequence selected from the group consisting of SEQ Identifiers: A251-A252 of Table 3A. In some embodiments, the PEgRNA comprises a PBS comprising a sequence selected from the group consisting of SEQ Identifiers: B1062-B1070 of Table 3B. In some embodiments, the PEgRNA comprises an editing template comprising a sequence selected from the group consisting of SEQ Identifiers: C3125-C3149 of Table 3C. In some embodiments, the prime editing composition further comprises a ngRNA that comprises a spacer comprising a sequence selected from the group consisting of SEQ Identifiers: D376-D384, D471, D472, D489-D494, D507, D508, D544, D545, D555, and D585 of Table 3D.

In some embodiments, the PEgRNA comprises a spacer comprising a sequence selected from the group consisting of SEQ Identifiers: A273-A274 of Table 3A. In some embodiments, the PEgRNA comprises a PBS comprising a sequence selected from the group consisting of SEQ Identifiers: B1071-B1079 of Table 3B. In some embodiments, the PEgRNA comprises an editing template comprising a sequence selected from the group consisting of SEQ Identifiers: C3150-C3211 of Table 3C. In some embodiments, the prime editing composition further comprises a ngRNA that comprises a spacer comprising a sequence selected from the group consisting of SEQ Identifiers: D509, D510, D511, D512, D549, and D552 of Table 3D.

In some embodiments, the PEgRNA comprises a spacer comprising a sequence selected from the group consisting of SEQ Identifiers: A255-A256 of Table 3A. In some embodiments, the PEgRNA comprises a PBS comprising a sequence selected from the group consisting of SEQ Identifiers: B1080-B1088 of Table 3B. In some embodiments, the PEgRNA comprises an editing template comprising a sequence selected from the group consisting of SEQ Identifiers: C3212-C3232 of Table 3C. In some embodiments, the prime editing composition further comprises a ngRNA that comprises a spacer comprising a sequence selected from the group consisting of SEQ Identifiers: D376-D384, D471, D472, D489-D494, D507, D508, D544, D545, D555, D570, D585, and D586 of Table 3D.

In some embodiments, the PEgRNA comprises a spacer comprising a sequence selected from the group consisting of SEQ Identifiers: A257-A260 of Table 3A. In some embodiments, the PEgRNA comprises a PBS comprising a sequence selected from the group consisting of SEQ Identifiers: B1089-B1097 of Table 3B. In some embodiments, the PEgRNA comprises an editing template comprising a sequence selected from the group consisting of SEQ Identifiers: C3233-C3252 of Table 3C. In some embodiments, the prime editing composition further comprises a ngRNA that comprises a spacer comprising a sequence selected from the group consisting of SEQ Identifiers: D380-D384, D394, D395, D403, D404, D411, D412, D421, D422, D489-D495, D508, D510, D539, D540, D544, D545, D555, D570, D571, D589, and D590 of Table 3D.

In some embodiments, the PEgRNA comprises a spacer comprising the sequence of SEQ Identifier: A261 of Table 3A. In some embodiments, the PEgRNA comprises a PBS comprising a sequence selected from the group consisting of SEQ Identifiers: B1098-B1106 of Table 3B. In some embodiments, the PEgRNA comprises an editing template comprising a sequence selected from the group consisting of SEQ Identifiers: C3253-C3275 of Table 3C. In some embodiments, the prime editing composition further comprises a ngRNA that comprises a spacer comprising a sequence selected from the group consisting of SEQ Identifiers: D445, D446, D453-D457, D475-D486, and D556-D 567 of Table 3D.

In some embodiments, the PEgRNA comprises a spacer comprising a sequence selected from the group consisting of SEQ Identifiers: A262-A264 of Table 3A. In some embodiments, the PEgRNA comprises a PBS comprising a sequence selected from the group consisting of SEQ Identifiers: B1107-B1115 of Table 3B. In some embodiments, the PEgRNA comprises an editing template comprising a sequence selected from the group consisting of SEQ Identifiers: C3276-C3295 of Table 3C. In some embodiments, the PEgRNA comprises a spacer comprising a sequence selected from the group consisting of SEQ Identifiers: A275-A276 of Table 3A. In some embodiments, the PEgRNA comprises a PBS comprising a sequence selected from the group consisting of SEQ Identifiers: B1116-B1124 of Table 3B. In some embodiments, the PEgRNA comprises an editing template comprising a sequence selected from the group consisting of SEQ Identifiers: C3296-C3326 of Table 3C. In some embodiments, the prime editing composition further comprises a ngRNA that comprises a spacer comprising a sequence selected from the group consisting of SEQ Identifiers: D452, D454-D457, D473-D486, D556-D569, D572, and D591-D 595 of Table 3D.

In some embodiments, the PEgRNA comprises a spacer comprising the sequence of SEQ Identifier: A267 of Table 3A. In some embodiments, the PEgRNA comprises a PBS comprising a sequence selected from the group consisting of SEQ Identifiers: B1125-B1133 of Table 3B. In some embodiments, the PEgRNA comprises an editing template comprising a sequence selected from the group consisting of SEQ Identifiers: C3327-C3344 of Table 3C. In some embodiments, the prime editing composition further comprises a ngRNA that comprises a spacer comprising a sequence selected from the group consisting of SEQ Identifiers: D376-D384, D489-D494, D508, D544, D545, D555, and D570 of Table 3D.

In some embodiments, the editing target sequence is in exon 10 of the CFTR gene.

In some embodiments, the editing target sequence or the complementary sequence on the CFTR gene thereof comprises a mutation that encodes an amino acid substitution A455E relative to a wild type CFTR polypeptide set forth in SEQ IDENTIFIER: 19, and wherein the editing template encodes an intended nucleotide edit that corrects the mutation. In some embodiments, the editing target sequence or the complementary sequence on the CFTR gene thereof comprises a C→→A mutation (c.1364C>A) at position 117,548,795, a A→→C mutation (c.1355A>C) at position 117,548,786, a deletion of T (c.1358delT) at position 117,548,789, a deletion of TTG (c.1360_1362delTTG) at position 117,548,791 a deletion of GG (c.1365_1366delGG) at position 117,548,796, a G→→T mutation (c.1366G>T) at position 117,548,797, a deletion of G (c.1366delG) at position 117,548,797, a G→C mutation (c.1369G>C) at position 117,548,800, a deletion of G (c.1373delG) at position 117,548,804, and/or a G→→T mutation (c.1373G>T) at position 117,548,804 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, the editing template comprises a wild type CFTR gene sequence. Accordingly, in some embodiments, the newly synthesized single stranded DNA encoded by the editing template is capable of correcting the c.1364C>A mutation, the c.1355A>C mutation, the c.1358delT mutation, the c.1360_1362delTTG mutation, the c.1365_1366delGG mutation, the c.1366G>T mutation, the c.1366delG mutation, the c.1369G>C mutation, the c.1373delG mutation, and/or the c.1373G>T mutation in the target CFTR gene. In some embodiments, the newly synthesized single stranded DNA encoded by the editing template is capable of correcting all of the c.1364C>A mutation, the c.1355A>C mutation, the c.1358delT mutation, the c.1360_1362delTTG mutation, the c.1365_1366delGG mutation, the c.1366G>T mutation, the c.1366delG mutation, the c.1369G>C mutation, the c.1373delG mutation, and the c.1373G>T mutation in the target CFTR gene. In some embodiments, two or more of the c.1364C>A mutation, the c.1355A>C mutation, the c.1358delT mutation, the c.1360_1362delTTG mutation, the c.1365_1366delGG mutation, the c.1366G>T mutation, the c.1366delG mutation, the c.1369G>C mutation, the c.1373delG mutation, and the c.1373G>T mutation are located on the same CFTR gene. In some embodiments, the newly synthesized single stranded DNA encoded by the editing template is capable of correcting the c.1364C>A mutation, the c.1355A>C mutation, the c.1358delT mutation, the c.1360_1362delTTG mutation, the c.1365_1366delGG mutation, the c.1366G>T mutation, the c.1366delG mutation, the c.1369G>C mutation, the c.1373delG mutation, and the c.1373G>T mutation that are located on different CFTR genes. For example, in some embodiments, a subject may comprise two copies of CFTR genes, each comprising a different mutation selected from the group consisting of the c.1364C>A mutation, the c.1355A>C mutation, the c.1358delT mutation, the c.1360_1362delTTG mutation, the c.1365_1366delGG mutation, the c.1366G>T mutation, the c.1366delG mutation, the c.1369G>C mutation, the c.1373delG mutation, and the c.1373G>T mutation, all of which may be corrected by an editing template of a single PEgRNA. In some embodiments, the editing template is capable of correcting one or more, or all of the c.1364C>A mutation, the c.1355A>C mutation, the c.1358delT mutation, the c.1360_1362delTTG mutation, the c.1365_1366delGG mutation, the c.1366G>T mutation, the c.1366delG mutation, the c.1369G>C mutation, the c.1373delG mutation, and the c.1373G>T mutation that are in different subjects, wherein each subject may have one or more of the mutations.

In some embodiments, the PEgRNA comprises a spacer comprising a sequence selected from the group consisting of SEQ Identifiers: A277-A278 of Table 3A. In some embodiments, the PEgRNA comprises a PBS comprising a sequence selected from the group consisting of SEQ Identifiers: B1134-B1142 of Table 3B. In some embodiments, the PEgRNA comprises an editing template comprising a sequence selected from the group consisting of SEQ Identifiers: C3345-C3374 of Table 3C. In some embodiments, the prime editing composition further comprises a ngRNA that comprises a spacer comprising a sequence selected from the group consisting of SEQ Identifiers: D596-D619 of Table 3D.

In some embodiments, the PEgRNA comprises a spacer comprising the sequence of SEQ Identifiers: A279 of Table 3A. In some embodiments, the PEgRNA comprises a PBS comprising a sequence selected from the group consisting of SEQ Identifiers: B1143-B1151 of Table 3B. In some embodiments, the PEgRNA comprises an editing template comprising a sequence selected from the group consisting of SEQ Identifiers: C3375-C3388 of Table 3C. In some embodiments, the prime editing composition further comprises a ngRNA that comprises a spacer comprising a sequence selected from the group consisting of SEQ Identifiers: D620-D643 of Table 3D.

In some embodiments, the PEgRNA comprises a spacer comprising a sequence selected from the group consisting of SEQ Identifiers: A280-A281 of Table 3A. In some embodiments, the PEgRNA comprises a PBS comprising a sequence selected from the group consisting of SEQ Identifiers: B1152-B1160 of Table 3B. In some embodiments, the PEgRNA comprises an editing template comprising a sequence selected from the group consisting of SEQ Identifiers: C3389-C3404 of Table 3C. In some embodiments, the prime editing composition further comprises a ngRNA that comprises a spacer comprising a sequence selected from the group consisting of SEQ Identifiers: D644-D673 of Table 3D.

In some embodiments, the PEgRNA comprises a spacer comprising a sequence selected from the group consisting of SEQ Identifiers: A282-A283 of Table 3A. In some embodiments, the PEgRNA comprises a PBS comprising a sequence selected from the group consisting of SEQ Identifiers: B1161-B1169 of Table 3B. In some embodiments, the PEgRNA comprises an editing template comprising a sequence selected from the group consisting of SEQ Identifiers: C3405-C3448 of Table 3C. In some embodiments, the prime editing composition further comprises a ngRNA that comprises a spacer comprising a sequence selected from the group consisting of SEQ Identifiers: D620-D641, and D674-D 688 of Table 3D.

In some embodiments, the PEgRNA comprises a spacer comprising a sequence selected from the group consisting of SEQ Identifiers: A284-A287 of Table 3A. In some embodiments, the PEgRNA comprises a PBS comprising a sequence selected from the group consisting of SEQ Identifiers: B1170-B1178 of Table 3B. In some embodiments, the PEgRNA comprises an editing template comprising a sequence selected from the group consisting of SEQ Identifiers: C3449-C3479 of Table 3C. In some embodiments, the prime editing composition further comprises a ngRNA that comprises a spacer comprising a sequence selected from the group consisting of SEQ Identifiers: D596-D619, and D689-D694 of Table 3D.

In some embodiments, the PEgRNA comprises a spacer comprising a sequence selected from the group consisting of SEQ Identifiers: A288-A289 of Table 3A. In some embodiments, the PEgRNA comprises a PBS comprising a sequence selected from the group consisting of SEQ Identifiers: B1179-B1187 of Table 3B. In some embodiments, the PEgRNA comprises an editing template comprising a sequence selected from the group consisting of SEQ Identifiers: C3480-C3517 of Table 3C. In some embodiments, the prime editing composition further comprises a ngRNA that comprises a spacer comprising a sequence selected from the group consisting of SEQ Identifiers: D695-D718 of Table 3D.

In some embodiments, the PEgRNA comprises a spacer comprising a sequence selected from the group consisting of SEQ ID Nos: A288-A289 of Table 3A. In some embodiments, the PEgRNA comprises a PBS comprising a sequence selected from the group consisting of SEQ ID Nos: B1179-B1187 of Table 3B. In some embodiments, the PEgRNA comprises an editing template comprising a sequence selected from the group consisting of SEQ ID Nos: C3480-C3517 of Table 3C. In some embodiments, the prime editing composition further comprises a ngRNA that comprises a spacer comprising a sequence selected from the group consisting of SEQ ID Nos: D695-D718 of Table 3D.

In some embodiments, the PEgRNA comprises a spacer comprising the sequence of SEQ Identifiers: A290 of Table 3A. In some embodiments, the PEgRNA comprises a spacer comprising a sequence as set forth in SEQ ID NO. 638 of Table 3A. In some embodiments, the PEgRNA comprises a PBS comprising a sequence selected from the group consisting of SEQ Identifiers: B1188-B1196 of Table 3B. In some embodiments, the PEgRNA comprises a PBS comprising a sequence selected from the group consisting of SEQ ID Nos: 2137-2143 of Table 3B. In some embodiments, the PEgRNA comprises a PBS comprising a sequence selected from the group consisting of sequences GCAGGCAA and GCAGGCAAG as disclosed in Table 3B. In some embodiments, the PEgRNA comprises an editing template comprising a sequence selected from the group consisting of SEQ Identifiers: C3518-C3537 of Table 3C. In some embodiments, the PEgRNA comprises an editing template comprising a sequence selected from the group consisting of SEQ ID Nos: 7544-7563 of Table 3C. In some embodiments, the prime editing composition further comprises a ngRNA that comprises a spacer comprising a sequence selected from the group consisting of SEQ Identifiers: D620-D642, D676, and D677 of Table 3D. In some embodiments, the prime editing composition further comprises a ngRNA that comprises a spacer comprising a sequence selected from the group consisting of SEQ ID Nos: 20253-20275 and 20309-20310 of Table 3D.

In some embodiments, the PEgRNA comprises a spacer comprising a sequence selected from the group consisting of SEQ Identifiers: A291-A293 of Table 3A. In some embodiments, the PEgRNA comprises a spacer comprising a sequence as set forth in SEQ ID NOs. 639-641 of Table 3A. In some embodiments, the PEgRNA comprises a PBS comprising a sequence selected from the group consisting of SEQ Identifiers: B1197-B1205 of Table 3B. In some embodiments, the PEgRNA comprises a PBS comprising a sequence selected from the group consisting of SEQ ID Nos: 2144-2150 of Table 3B. In some embodiments, the PEgRNA comprises a PBS comprising a sequence selected from the group consisting of sequences CTGTCCTC and CTGTCCTCT as disclosed in Table 3B. In some embodiments, the PEgRNA comprises an editing template comprising a sequence selected from the group consisting of SEQ Identifiers: C3538-C3568 of Table 3C. In some embodiments, the PEgRNA comprises an editing template comprising a sequence selected from the group consisting of SEQ ID Nos: 7564-7594 of Table 3C. In some embodiments, the prime editing composition further comprises a ngRNA that comprises a spacer comprising a sequence selected from the group consisting of SEQ Identifiers: D610, D617, D619, and D689-D 694 of Table 3D. In some embodiments, the prime editing composition further comprises a ngRNA that comprises a spacer comprising a sequence selected from the group consisting of SEQ ID Nos: 20243, 20250, 20252 and 20322-20327 of Table 3D.

In some embodiments, the PEgRNA comprises a spacer comprising a sequence selected from the group consisting of SEQ Identifiers: A294-A295 of Table 3A. In some embodiments, the PEgRNA comprises a spacer comprising a sequence as set forth in SEQ ID NOs. 642-643 of Table 3A. In some embodiments, the PEgRNA comprises a PBS comprising a sequence selected from the group consisting of SEQ Identifiers: B1206-B1214 of Table 3B. In some embodiments, the PEgRNA comprises a PBS comprising a sequence selected from the group consisting of SEQ ID Nos: 2151-2157 of Table 3B. In some embodiments, the PEgRNA comprises a PBS comprising a sequence selected from the group consisting of sequences ACTGTCCT and ACTGTCCTC as disclosed in Table 3B. In some embodiments, the PEgRNA comprises an editing template comprising a sequence selected from the group consisting of SEQ Identifiers: C3569-C3630 of Table 3C. In some embodiments, the PEgRNA comprises an editing template comprising a sequence selected from the group consisting of SEQ ID Nos: 7595-7656 of Table 3C. In some embodiments, the prime editing composition further comprises a ngRNA that comprises a spacer comprising a sequence selected from the group consisting of SEQ Identifiers: D644-D664, and D719-D722 of Table 3D. In some embodiments, the prime editing composition further comprises a ngRNA that comprises a spacer comprising a sequence selected from the group consisting of SEQ ID Nos: 20555 and 20654-20658 of Table 3D.

In some embodiments, the PEgRNA comprises a spacer comprising a sequence selected from the group consisting of SEQ ID Nos: A296-A297 of Table 3A. In some embodiments, the PEgRNA comprises a spacer comprising a sequence as set forth in SEQ ID NOs. 644-645 of Table 3A. In some embodiments, the PEgRNA comprises a PBS comprising a sequence selected from the group consisting of SEQ Identifiers: B1215-B1223 of Table 3B. In some embodiments, the PEgRNA comprises a PBS comprising a sequence selected from the group consisting of SEQ ID Nos: 2158-2164 of Table 3B. In some embodiments, the PEgRNA comprises a PBS comprising a sequence selected from the group consisting of sequences ATCTTGAA and ATCTTGAAA as disclosed in Table 3B. In some embodiments, the PEgRNA comprises an editing template comprising a sequence selected from the group consisting of SEQ Identifiers: C3631-C3668 of Table 3C. In some embodiments, the PEgRNA comprises an editing template comprising a sequence selected from the group consisting of SEQ ID Nos: 7657-7714 of Table 3C. In some embodiments, the prime editing composition further comprises a ngRNA that comprises a spacer comprising a sequence selected from the group consisting of SEQ Identifiers: D596-D617, and D619 of Table 3D. In some embodiments, the prime editing composition further comprises a ngRNA that comprises a spacer comprising a sequence selected from the group consisting of SEQ ID Nos: 20229-20250 and 20252 of Table 3D.

In some embodiments, the PEgRNA comprises a spacer comprising a sequence selected from the group consisting of SEQ Identifiers: A298-A301 of Table 3A. In some embodiments, the PEgRNA comprises a spacer comprising a sequence as set forth in SEQ ID NOs. 646-649 of Table 3A. In some embodiments, the PEgRNA comprises a PBS comprising a sequence selected from the group consisting of SEQ Identifiers: B1224-B1232 of Table 3B. In some embodiments, the PEgRNA comprises a PBS comprising a sequence selected from the group consisting of SEQ ID Nos: 2165-2171 of Table 3B. In some embodiments, the PEgRNA comprises a PBS comprising a sequence selected from the group consisting of sequences CTATCTTG and CTATCTTGA as disclosed in Table 3B. In some embodiments, the PEgRNA comprises an editing template comprising a sequence selected from the group consisting of SEQ Identifiers: C3669-C3710 of Table 3C. In some embodiments, the PEgRNA comprises an editing template comprising a sequence selected from the group consisting of SEQ ID Nos: 7695-7736 of Table 3C. In some embodiments, the prime editing composition further comprises a ngRNA that comprises a spacer comprising a sequence selected from the group consisting of SEQ Identifiers: D596-D619, and D689-D694 of Table 3D. In some embodiments, the prime editing composition further comprises a ngRNA that comprises a spacer comprising a sequence selected from the group consisting of SEQ ID Nos: 20229-20252 and 20322-20327 of Table 3D.

In some embodiments, the PEgRNA comprises a spacer comprising the sequence of SEQ Identifiers: A302 of Table 3A. In some embodiments, the PEgRNA comprises a spacer comprising a sequence as set forth in SEQ ID NO. 650 of Table 3A. In some embodiments, the PEgRNA comprises a PBS comprising a sequence selected from the group consisting of SEQ Identifiers: B1233-B1241 of Table 3B. In some embodiments, the PEgRNA comprises a PBS comprising a sequence selected from the group consisting of SEQ ID Nos: 2172-2178 of Table 3B. In some embodiments, the PEgRNA comprises a PBS comprising a sequence selected from the group consisting of sequences GTCCTCTT and GTCCTCTTT as disclosed in Table 3B. In some embodiments, the PEgRNA comprises an editing template comprising a sequence selected from the group consisting of SEQ Identifiers: C3711-C3740 of Table 3C. In some embodiments, the PEgRNA comprises an editing template comprising a sequence selected from the group consisting of SEQ ID Nos: 7737-7766 of Table 3C. In some embodiments, the prime editing composition further comprises a ngRNA that comprises a spacer comprising a sequence selected from the group consisting of SEQ Identifiers: D644-D664, and D722 of Table 3D. In some embodiments, the prime editing composition further comprises a ngRNA that comprises a spacer comprising a sequence selected from the group consisting of SEQ ID Nos: 20277-20297 and 20405 of Table 3D.

In some embodiments, the PEgRNA comprises a spacer comprising a sequence selected from the group consisting of SEQ Identifiers: A303-A304 of Table 3A. In some embodiments, the PEgRNA comprises a spacer comprising a sequence as set forth in SEQ ID NOs. 651-652 of Table 3A. In some embodiments, the PEgRNA comprises a PBS comprising a sequence selected from the group consisting of SEQ Identifiers: B1242-B1250 of Table 3B. In some embodiments, the PEgRNA comprises a PBS comprising a sequence selected from the group consisting of SEQ ID Nos: 2179-2185 of Table 3B. In some embodiments, the PEgRNA comprises a PBS comprising a sequence selected from the group consisting of sequences ATTAATAT and ATTAATATC as disclosed in Table 3B. In some embodiments, the PEgRNA comprises an editing template comprising a sequence selected from the group consisting of SEQ Identifiers: C3741-C3762 of Table 3C. In some embodiments, the PEgRNA comprises an editing template comprising a sequence selected from the group consisting of SEQ ID Nos: 7767-7788 of Table 3C. In some embodiments, the prime editing composition further comprises a ngRNA that comprises a spacer comprising a sequence selected from the group consisting of SEQ Identifiers: D596-D619, D723, and D724 of Table 3D. In some embodiments, the prime editing composition further comprises a ngRNA that comprises a spacer comprising a sequence selected from the group consisting of SEQ ID Nos: 20229-20252, and 20356-20357 of Table 3D.

In some embodiments, the PEgRNA comprises a spacer comprising a sequence selected from the group consisting of SEQ Identifiers: A305-A306 of Table 3A. In some embodiments, the PEgRNA comprises a spacer comprising a sequence as set forth in SEQ ID NOs. 653-654 of Table 3A. In some embodiments, the PEgRNA comprises a PBS comprising a sequence selected from the group consisting of SEQ Identifiers: B1251-B1259 of Table 3B. In some embodiments, the PEgRNA comprises a PBS comprising a sequence selected from the group consisting of SEQ ID Nos: 2186-2189 of Table 3B. In some embodiments, the PEgRNA comprises a PBS comprising a sequence selected from the group consisting of sequences GGAGCAGG and GGAGCAGGC as disclosed in Table 3B. In some embodiments, the PEgRNA comprises an editing template comprising a sequence selected from the group consisting of SEQ Identifiers: C3763-C3785 of Table 3C. In some embodiments, the PEgRNA comprises an editing template comprising a sequence selected from the group consisting of SEQ ID Nos: 7789-7811 of Table 3C. In some embodiments, the prime editing composition further comprises a ngRNA that comprises a spacer comprising a sequence selected from the group consisting of SEQ Identifiers: D620-D640, D674-D677, and D725-D729 of Table 3D. In some embodiments, the prime editing composition further comprises a ngRNA that comprises a spacer comprising a sequence selected from the group consisting of SEQ ID Nos: 20253-20273, 20307-20310, and 20358-20362 of Table 3D.

In some embodiments, the PEgRNA comprises a spacer comprising a sequence selected from the group consisting of SEQ Identifiers: A307-A308 of Table 3A. In some embodiments, the PEgRNA comprises a spacer comprising a sequence as set forth in SEQ ID NOs. 655-656 of Table 3A. In some embodiments, the PEgRNA comprises a PBS comprising a sequence selected from the group consisting of SEQ Identifiers: B1260-B1268 of Table 3B. In some embodiments, the PEgRNA comprises a PBS comprising a sequence selected from the group consisting of SEQ ID Nos: 2193-2199 of Table 3B. In some embodiments, the PEgRNA comprises a PBS comprising a sequence selected from the group consisting of sequences AATTAATA and AATTAATAT as disclosed in Table 3B. In some embodiments, the PEgRNA comprises an editing template comprising a sequence selected from the group consisting of SEQ Identifiers: C3786-C3797 of Table 3C. In some embodiments, the PEgRNA comprises an editing template comprising a sequence selected from the group consisting of SEQ ID Nos: 7812-7823 of Table 3C. In some embodiments, the prime editing composition further comprises a ngRNA that comprises a spacer comprising a sequence selected from the group consisting of SEQ Identifiers: D644-D673 of Table 3D. In some embodiments, the prime editing composition further comprises a ngRNA that comprises a spacer comprising a sequence selected from the group consisting of SEQ ID Nos: 20277-20306 of Table 3D.

In some embodiments, the PEgRNA comprises a spacer comprising a sequence selected from the group consisting of SEQ Identifiers: A309-A310 of Table 3A. In some embodiments, the PEgRNA comprises a spacer comprising a sequence as set forth in SEQ ID NOs. 657-658 of Table 3A. In some embodiments, the PEgRNA comprises a PBS comprising a sequence selected from the group consisting of SEQ Identifiers: B1269-B1277 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C3798-C3859 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D645-D664, D719-D722, and D730 of Table 3D. In some embodiments, the prime editing composition further comprises a ngRNA that comprises a spacer comprising a sequence selected from the group consisting of SEQ ID Nos: 20278-20297, 20352-20355, and 20363 of Table 3D.

In some embodiments, the PEgRNA comprises a spacer comprising the sequence of SEQ Identifiers: A311 of Table 3A. In some embodiments, the PEgRNA comprises a spacer comprising a sequence as set forth in SEQ ID NO. 659 of Table 3A. In some embodiments, the PEgRNA comprises a PBS comprising a sequence selected from the group consisting of SEQ Identifiers: B1278-B1286 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C3860-C3890 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D645-D664, D722, and D730 of Table 3D. In some embodiments, the prime editing composition further comprises a ngRNA that comprises a spacer comprising a sequence selected from the group consisting of SEQ ID Nos: 20278-20297, 20355, and 20363 of Table 3D.

In some embodiments, the PEgRNA comprises a spacer comprising a sequence selected from the group consisting of SEQ Identifiers: A312-A314 of Table 3A. In some embodiments, the PEgRNA comprises a spacer comprising a sequence as set forth in SEQ ID NOs. 660-662 of Table 3A. In some embodiments, the PEgRNA comprises a PBS comprising a sequence selected from the group consisting of SEQ Identifiers: B1287-B1295 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C3891-C3911 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D700, D703, D704, D705, and D731-D747 of Table 3D. In some embodiments, the prime editing composition further comprises a ngRNA that comprises a spacer comprising a sequence selected from the group consisting of SEQ ID Nos: 20333, 20336-20338, and 20364-20380 of Table 3D.

In some embodiments, the PEgRNA comprises a spacer comprising the sequence of SEQ Identifiers: A315 of Table 3A. In some embodiments, the PEgRNA comprises a spacer comprising a sequence as set forth in SEQ ID NO. 663 of Table 3A. In some embodiments, the PEgRNA comprises a PBS comprising a sequence selected from the group consisting of SEQ Identifiers: B1296-B1304 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C3912-C3921 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D621-D643 of Table 3D. In some embodiments, the prime editing composition further comprises a ngRNA that comprises a spacer comprising a sequence selected from the group consisting of SEQ ID Nos: 20254-20276 of Table 3D.

In some embodiments, the PEgRNA comprises a spacer comprising a sequence selected from the group consisting of SEQ Identifiers: A316-A317 of Table 3A. In some embodiments, the PEgRNA comprises a spacer comprising a sequence as set forth in SEQ ID NOs. 664-665 of Table 3A. In some embodiments, the PEgRNA comprises a PBS comprising a sequence selected from the group consisting of SEQ Identifiers: B1305-B1313 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C3922-C3944 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D644-D722, and D748 of Table 3D. In some embodiments, the prime editing composition further comprises a ngRNA that comprises a spacer comprising a sequence selected from the group consisting of SEQ ID Nos: 20277-20355 and 20381 of Table 3D.

In some embodiments, the PEgRNA comprises a spacer comprising a sequence selected from the group consisting of SEQ Identifiers: A318-A319 of Table 3A. In some embodiments, the PEgRNA comprises a spacer comprising a sequence as set forth in SEQ ID NOs. 666-667 of Table 3A. In some embodiments, the PEgRNA comprises a PBS comprising a sequence selected from the group consisting of SEQ Identifiers: B1314-B1322 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C3945-C3975 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D644-D673, D722, and D748 of Table 3D. In some embodiments, the prime editing composition further comprises a ngRNA that comprises a spacer comprising a sequence selected from the group consisting of SEQ ID Nos: 20272-20306, 20355 and 20381 of Table 3D.

In some embodiments, the PEgRNA comprises a spacer comprising a sequence selected from the group consisting of SEQ Identifiers: A320-A321 of Table 3A. In some embodiments, the PEgRNA comprises a spacer comprising a sequence as set forth in SEQ ID NOs. 668-669 of Table 3A. In some embodiments, the PEgRNA comprises a PBS comprising a sequence selected from the group consisting of SEQ Identifiers: B1323-B1331 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C3976-C3995 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D644-D673, D722, and D748 of Table 3D. In some embodiments, the prime editing composition further comprises a ngRNA that comprises a spacer comprising a sequence selected from the group consisting of SEQ ID Nos: 20277-20306, 20355, and 20381 of Table 3D.

In some embodiments, the PEgRNA comprises a spacer comprising a sequence selected from the group consisting of SEQ Identifiers: A322-A323 of Table 3A. In some embodiments, the PEgRNA comprises a spacer comprising a sequence as set forth in SEQ ID NOs. 670-671 of Table 3A. In some embodiments, the PEgRNA comprises a PBS comprising a sequence selected from the group consisting of SEQ Identifiers: B1332-B1340 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C3996-C4017 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D644-D664, and D719-D722 of Table 3D. In some embodiments, the prime editing composition further comprises a ngRNA that comprises a spacer comprising a sequence selected from the group consisting of SEQ ID Nos: 20277-20297 and 20352-20355 of Table 3D.

In some embodiments, the PEgRNA comprises a spacer comprising a sequence selected from the group consisting of SEQ Identifiers: A324-A325 of Table 3A. In some embodiments, the PEgRNA comprises a spacer comprising a sequence as set forth in SEQ ID NOs. 672-673 of Table 3A. In some embodiments, the PEgRNA comprises a PBS comprising a sequence selected from the group consisting of SEQ Identifiers: B1341-B1349 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4018-C4073 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D697-D702, D705, D706, D709-D718, and D749-D758 of Table 3D. In some embodiments, the prime editing composition further comprises a ngRNA that comprises a spacer comprising a sequence selected from the group consisting of SEQ ID Nos: 20330-20335, 20338-20339, and 20382-20391 of Table 3D.

In some embodiments, the PEgRNA comprises a spacer comprising the sequence of SEQ Identifiers: A326 of Table 3A. In some embodiments, the PEgRNA comprises a spacer comprising a sequence as set forth in SEQ ID NO. 674 of Table 3A. In some embodiments, the PEgRNA comprises a PBS comprising a sequence selected from the group consisting of SEQ Identifiers: B1350-B1358 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4074-C4102 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D620-D639, D674-D677, D759, D760, and D761 of Table 3D. In some embodiments, the prime editing composition further comprises a ngRNA that comprises a spacer comprising a sequence selected from the group consisting of SEQ ID Nos: 20253-20272, 20307-20310, and 20392-20394 of Table 3D.

In some embodiments, the PEgRNA comprises a spacer comprising a sequence selected from the group consisting of SEQ Identifiers: A327-A328 of Table 3A. In some embodiments, the PEgRNA comprises a spacer comprising a sequence as set forth in SEQ ID NOs. 675-676 of Table 3A. In some embodiments, the PEgRNA comprises a PBS comprising a sequence selected from the group consisting of SEQ Identifiers: B1359-B1367 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4103-C4154 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D596-D617, and D619 of Table 3D. In some embodiments, the prime editing composition further comprises a ngRNA that comprises a spacer comprising a sequence selected from the group consisting of SEQ ID Nos: 20229-20250 and 20252 of Table 3D.

In some embodiments, the PEgRNA comprises a spacer comprising the sequence of SEQ Identifiers: A329 of Table 3A. In some embodiments, the PEgRNA comprises a spacer comprising a sequence as set forth in SEQ ID NO. 677 of Table 3A. In some embodiments, the PEgRNA comprises a PBS comprising a sequence selected from the group consisting of SEQ Identifiers: B1368-B1376 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4155-C4181 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D644-D664 and D722 of Table 3D. In some embodiments, the prime editing composition further comprises a ngRNA that comprises a spacer comprising a sequence selected from the group consisting of SEQ ID Nos: 20277-20297 and 20355 of Table 3D.

In some embodiments, the editing target sequence is in exon 11 of the CFTR gene.

In some embodiments, the editing target sequence or the complementary sequence on the CFTR gene thereof comprises a mutation that encodes an amino acid substitution E504Q, E504*, F508C, N505H, I506L, I506S, I506M, I507N, F508I, G509R, deletion of 1507, and/or deletion of 1508 relative to a wild type CFTR polypeptide set forth in SEQ ID NO: 19, and wherein the editing template encodes an intended nucleotide edit that corrects the mutation. In some embodiments, the editing target sequence or the complementary sequence on the CFTR gene thereof comprises a deletion of nucleotides ATC (c.1519_1521delATC) at position 117,559,590, a deletion of nucleotides AG and an insertion of T (c.1509_1510delAGinsT) at position 117,559,580, a G→→C mutation (c.1510G>C) at position 117,559,581, a G→→T mutation (c.1510G>T) at position 117,559,581, a deletion of nucleotides CTT (c.1521_1523delCTT) at position 117,559,592, a T→→G mutation (c.1523T>G) at position 117,559,594, a A→→C mutation (c.1513A>C) at position 117,559,584, a deletion of A (c.1514delA) at position 117,559,585, a A→→C mutation (c.1516A>C) at position 117,559,587, a T→→G mutation (c.1517T>G) at position 117,559,588, a C→→G mutation (c.1518C>G) at position 117,559,589, a duplication of nucleotides ATCAT (c.1516_1520dupATCAT) at position 117,559,591, a T→A mutation (c.1520T>A) at position 117,559,591, a T-A mutation (c.1522T>A) at position 117,559,593, a G→C mutation (c.1526G>C) at position 117,559,597, a deletion of G (c.1528delG) at position 117,559,599, and/or a deletion of TT (c.1530_1531delTT) at position 117,559,601 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide. In some embodiments, the editing template comprises a wild type CFTR gene sequence. Accordingly, in some embodiments, the newly synthesized single stranded DNA encoded by the editing template is capable of correcting the c.1519_1521delATC mutation, the c.1509_1510delAGinsT mutation, the c.1510G>C mutation, the c.1510G>T mutation, c.1521_1523delCTT mutation, the c.1523T>G mutation, the c.1513A>C mutation, the c.1514delA mutation, the c.1516A>C mutation, the c.1517T>G mutation, the c.1518C>G mutation, the c.1516_1520dupATCAT mutation, the c.1520T>A mutation, the c.1522T>A mutation, the c.1525G>C mutation, the c.1526G>C mutation, the c.1528delG mutation, and/or the c.1530_1531delTT mutation in the target CFTR gene. In some embodiments, the newly synthesized single stranded DNA encoded by the editing template is capable of correcting all of the c.1519_1521delATC mutation, the c.1509_1510delAGinsT mutation, the c.1510G>C mutation, the c.1510G>T mutation, c.1521_1523delCTT mutation, the c.1523T>G mutation, the c.1513A>C mutation, the c.1514delA mutation, the c.1516A>C mutation, the c.1517T>G mutation, the c.1518C>G mutation, the c.1516_1520dupATCAT mutation, the c.1520T>A mutation, the c.1522T>A mutation, the c.1525G>C mutation, the c.1526G>C mutation, the c.1528delG mutation, and the c.1530_1531delTT mutation in the target CFTR gene. In some embodiments, two or more of the c.1519_1521delATC mutation, the c.1509_1510delAGinsT mutation, the c.1510G>C mutation, the c.1510G>T mutation, c.1521_1523delCTT mutation, the c.1523T>G mutation, the c.1513A>C mutation, the c.1514delA mutation, the c.1516A>C mutation, the c.1517T>G mutation, the c.1518C>G mutation, the c.1516_1520dupATCAT mutation, the c.1520T>A mutation, the c.1522T>A mutation, the c.1525G>C mutation, the c.1526G>C mutation, the c.1528delG mutation, and the c.1530_1531delTT mutation are located on the same CFTR gene. In some embodiments, the newly synthesized single stranded DNA encoded by the editing template is capable of correcting the c.1519_1521delATC mutation, the c.1509_1510delAGinsT mutation, the c.1510G>C mutation, the c.1510G>T mutation, c.1521_1523delCTT mutation, the c.1523T>G mutation, the c.1513A>C mutation, the c.1514delA mutation, the c.1516A>C mutation, the c.1517T>G mutation, the c.1518C>G mutation, the c.1516_1520dupATCAT mutation, the c.1520T>A mutation, the c.1522T>A mutation, the c.1525G>C mutation, the c.1526G>C mutation, the c.1528delG mutation, and/or the c.1530_1531delTT mutation that are located on different CFTR genes. For example, in some embodiments, a subject may comprise two copies of CFTR genes, each comprising a different mutation selected from the group consisting of the c.1519_1521delATC mutation, the c.1509_1510delAGinsT mutation, the c.1510G>C mutation, the c.1510G>T mutation, c.1521_1523delCTT mutation, the c.1523T>G mutation, the c.1513A>C mutation, the c.1514delA mutation, the c.1516A>C mutation, the c.1517T>G mutation, the c.1518C>G mutation, the c.1516_1520dupATCAT mutation, the c.1520T>A mutation, the c.1522T>A mutation, the c.1525G>C mutation, the c.1526G>C mutation, the c.1528delG mutation, and the c.1530_1531delTT mutation, all of which may be corrected by an editing template of a single PEgRNA. In some embodiments, the editing template is capable of correcting one or more, or all of the c.1519_1521delATC mutation, the c.1509_1510delAGinsT mutation, the c.1510G>C mutation, the c.1510G>T mutation, c.1521_1523delCTT mutation, the c.1523T>G mutation, the c.1513A>C mutation, the c.1514delA mutation, the c.1516A>C mutation, the c.1517T>G mutation, the c.1518C>G mutation, the c.1516_1520dupATCAT mutation, the c.1520T>A mutation, the c.1522T>A mutation, the c.1525G>C mutation, the c.1526G>C mutation, the c.1528delG mutation, and the c.1530_1531delTT mutation that are in different subjects, wherein each subject may have one or more of the mutations.

In some embodiments, the PEgRNA comprises a spacer comprising a sequence selected from the group consisting of SEQ Identifiers: A330-A331 of Table 3A. In some embodiments, the PEgRNA comprises a spacer comprising a sequence as set forth in SEQ ID NOs. 678-679 of Table 3A. In some embodiments, the PEgRNA comprises a PBS comprising a sequence selected from the group consisting of SEQ Identifiers: B1377-B1385 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4592-C4601 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D762-D782 of Table 3D. In some embodiments, the prime editing composition further comprises a ngRNA that comprises a spacer comprising a sequence selected from the group consisting of SEQ ID Nos: 20395-20415 of Table 3D.

In some embodiments, the PEgRNA comprises a spacer comprising the sequence of SEQ Identifiers: A332 of Table 3A. In some embodiments, the PEgRNA comprises a spacer comprising a sequence as set forth in SEQ ID NO. 680 of Table 3A. In some embodiments, the PEgRNA comprises a PBS comprising a sequence selected from the group consisting of SEQ Identifiers: B1386-B1394 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4602-C4612 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D765-D782 of Table 3D. In some embodiments, the prime editing composition further comprises a ngRNA that comprises a spacer comprising a sequence selected from the group consisting of SEQ ID Nos: 20398-20715 of Table 3D.

In some embodiments, the PEgRNA comprises a spacer comprising the sequence of SEQ Identifiers: A364 of Table 3A. In some embodiments, the PEgRNA comprises a spacer comprising a sequence as set forth in SEQ ID NO. 712 of Table 3A. In some embodiments, the PEgRNA comprises a PBS comprising a sequence selected from the group consisting of SEQ Identifiers: B1530-B1538. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4613-C4643. In some embodiments, the prime editing composition further comprises a ngRNA that comprises a spacer comprising a sequence selected from the group consisting of SEQ Identifiers: D768-D782, D813, D814, D890, D891, and D892 of Table 3D. In some embodiments, the prime editing composition further comprises a ngRNA that comprises a spacer comprising a sequence selected from the group consisting of SEQ ID Nos: 20401-20415, 20446-20447, and 20523-20525 of Table 3D.

In some embodiments, the PEgRNA comprises a spacer comprising a sequence selected from the group consisting of SEQ Identifiers: A333-A334 of Table 3A. In some embodiments, the PEgRNA comprises a spacer comprising a sequence as set forth in SEQ ID NOs. 681-682 of Table 3A. In some embodiments, the PEgRNA comprises a PBS comprising a sequence selected from the group consisting of SEQ Identifiers: B1395-B1403 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4644-C4685 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D783-D803 of Table 3D. In some embodiments, the prime editing composition further comprises a ngRNA that comprises a spacer comprising a sequence selected from the group consisting of SEQ ID Nos: 20555 and 20654-20658 of Table 3D.

In some embodiments, the PEgRNA comprises a spacer comprising a sequence selected from the group consisting of SEQ Identifiers: A365-A367 of Table 3A. In some embodiments, the PEgRNA comprises a spacer comprising a sequence as set forth in SEQ ID NOs. 713-715 of Table 3A. In some embodiments, the PEgRNA comprises a PBS comprising a sequence selected from the group consisting of SEQ Identifiers: B1413-B1421 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4686-C4716 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D797, D802, D803, D805, D806, D809, D810, and D893-D897 of Table 3D. In some embodiments, the prime editing composition further comprises a ngRNA that comprises a spacer comprising a sequence selected from the group consisting of SEQ ID Nos: 20430, 20435-20436, 20438-20439, 20442-20443, and 20526-20530 of Table 3D.

In some embodiments, the PEgRNA comprises a spacer comprising a sequence selected from the group consisting of SEQ Identifiers: A368-A369 of Table 3A. In some embodiments, the PEgRNA comprises a spacer comprising a sequence as set forth in SEQ ID NOs. 716-717 of Table 3A. In some embodiments, the PEgRNA comprises a PBS comprising a sequence selected from the group consisting of SEQ Identifiers: B1422-B1430 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4717-C4747 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D763, D764, D767-D782, D813, D814, D818, D890, D891, D892, and D898 of Table 3D. In some embodiments, the prime editing composition further comprises a ngRNA that comprises a spacer comprising a sequence selected from the group consisting of SEQ ID Nos: 20396-20397, 20400-20415, 20446-20447, 20451, 20523-20525, and 20531 of Table 3D.

In some embodiments, the PEgRNA comprises a spacer comprising the sequence of SEQ Identifiers: A370 of Table 3A. In some embodiments, the PEgRNA comprises a spacer comprising a sequence as set forth in SEQ ID NO. 718 of Table 3A. In some embodiments, the PEgRNA comprises a PBS comprising a sequence selected from the group consisting of SEQ v: B1431-B1439 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4748-C4778 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D767-D782, D813, 814, D890, D891, and D892 of Table 3D. In some embodiments, the prime editing composition further comprises a ngRNA that comprises a spacer comprising a sequence selected from the group consisting of SEQ ID Nos: 20400-20415, 20446-20447 and 20523-20525 of Table 3D.

In some embodiments, the PEgRNA comprises a spacer comprising a sequence selected from the group consisting of SEQ Identifiers: A343-A344 of Table 3A. In some embodiments, the PEgRNA comprises a spacer comprising a sequence as set forth in SEQ ID NOs. 691-692 of Table 3A. In some embodiments, the PEgRNA comprises a PBS comprising a sequence selected from the group consisting of SEQ Identifiers: B1440-B1448 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4779-C4824 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D820-D839 of Table 3D. In some embodiments, the prime editing composition further comprises a ngRNA that comprises a spacer comprising a sequence selected from the group consisting of SEQ ID Nos: 20453-20472 of Table 3D.

In some embodiments, the PEgRNA comprises a spacer comprising the sequence of SEQ Identifiers: A345 of Table 3A. In some embodiments, the PEgRNA comprises a spacer comprising a sequence as set forth in SEQ ID NO. 693 of Table 3A. In some embodiments, the PEgRNA comprises a PBS comprising a sequence selected from the group consisting of SEQ Identifiers: B1449-B1457 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4825-C4829 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D826-D840, and D899 of Table 3D. In some embodiments, the prime editing composition further comprises a ngRNA that comprises a spacer comprising a sequence selected from the group consisting of SEQ ID Nos: 20459-20473 and 20532 of Table 3D.

In some embodiments, the PEgRNA comprises a spacer comprising a sequence selected from the group consisting of SEQ Identifiers: A346-A348 of Table 3A. In some embodiments, the PEgRNA comprises a spacer comprising a sequence as set forth in SEQ ID NOs. 694-696 of Table 3A. In some embodiments, the PEgRNA comprises a PBS comprising a sequence selected from the group consisting of SEQ Identifiers: B1458-B1466 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4830-C4838 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D797, D802, D805, D806, D809, D810, D841, D842, and D843 of Table 3D. In some embodiments, the prime editing composition further comprises a ngRNA that comprises a spacer comprising a sequence selected from the group consisting of SEQ ID Nos: 20430, 20435-20436, 20438-20439, 20442-20443, and 20474-20476 of Table 3D.

In some embodiments, the PEgRNA comprises a spacer comprising a sequence selected from the group consisting of SEQ Identifiers: A349-A350 of Table 3A. In some embodiments, the PEgRNA comprises a spacer comprising a sequence as set forth in SEQ ID NOs. 697-698 of Table 3A. In some embodiments, the PEgRNA comprises a spacer comprising a sequence as set forth in SEQ ID NO. of Table 3A. In some embodiments, the PEgRNA comprises a PBS comprising a sequence selected from the group consisting of SEQ Identifiers: B1467-B1475 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4839-C4869 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D822-D837, D846-D853, and D900-D903 of Table 3D. In some embodiments, the prime editing composition further comprises a ngRNA that comprises a spacer comprising a sequence selected from the group consisting of SEQ ID Nos: 20455-20470, 20479-20486 and 20533-20536 of Table 3D.

In some embodiments, the PEgRNA comprises a spacer comprising a sequence selected from the group consisting of SEQ Identifiers: A351-A354 of Table 3A. In some embodiments, the PEgRNA comprises a spacer comprising a sequence as set forth in SEQ ID NOs. 699-702 of Table 3A. In some embodiments, the PEgRNA comprises a PBS comprising a sequence selected from the group consisting of SEQ Identifiers: B1476-B1484 of Table 3B. In some embodiments, the PEgRNA comprises an editing template comprising a sequence selected from the group consisting of SEQ Identifiers: C4870-C4913 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D856-D880 of Table 3D. In some embodiments, the prime editing composition further comprises a ngRNA that comprises a spacer comprising a sequence selected from the group consisting of SEQ ID Nos: 20489-20513 of Table 3D.

In some embodiments, the PEgRNA comprises a spacer comprising a sequence selected from the group consisting of SEQ Identifiers: A355-A356 of Table 3A. In some embodiments, the PEgRNA comprises a spacer comprising a sequence as set forth in SEQ ID NOs. 703-704 of Table 3A. In some embodiments, the PEgRNA comprises a PBS comprising a sequence selected from the group consisting of SEQ Identifiers: B1485-B1493 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4914-C4937 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D822-D839, and D846-D853 of Table 3D. In some embodiments, the prime editing composition further comprises a ngRNA that comprises a spacer comprising a sequence selected from the group consisting of SEQ ID Nos: 20455-20472 and 20479-20486 of Table 3D.

In some embodiments, the PEgRNA comprises a spacer comprising a sequence selected from the group consisting of SEQ Identifiers: A357-A358 of Table 3A. In some embodiments, the PEgRNA comprises a spacer comprising a sequence as set forth in SEQ ID NOs. 705-706 of Table 3A. In some embodiments, the PEgRNA comprises a PBS comprising a sequence selected from the group consisting of SEQ Identifiers: B1494-B1502 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4938-C4959 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D765-D782, D813, D814, and D881-D890 of Table 3D. In some embodiments, the prime editing composition further comprises a ngRNA that comprises a spacer comprising a sequence selected from the group consisting of SEQ ID Nos: 20398-20415, 20446-20447, and 20514-20523 of Table 3D.

In some embodiments, the PEgRNA comprises a spacer comprising the sequence of SEQ Identifier: A361 of Table 3A. In some embodiments, the PEgRNA comprises a spacer comprising a sequence as set forth in SEQ ID NO.709 of Table 3A. In some embodiments, the PEgRNA comprises a PBS comprising a sequence selected from the group consisting of SEQ Identifiers: B1512-B1520 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4960-C4965 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D765-D781 of Table 3D. In some embodiments, the prime editing composition further comprises a ngRNA that comprises a spacer comprising a sequence selected from the group consisting of SEQ ID Nos: 20398-20414 of Table 3D.

In some embodiments, the PEgRNA comprises a spacer comprising a sequence selected from the group consisting of SEQ Identifiers: A330-A331 of Table 3A. In some embodiments, the PEgRNA comprises a spacer comprising a sequence as set forth in SEQ ID NOs. 678-679 of Table 3A. In some embodiments, the PEgRNA comprises a PBS comprising a sequence selected from the group consisting of SEQ Identifiers: B1377-B1385 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4966-C4975 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D762-D782 of Table 3D. In some embodiments, the prime editing composition further comprises a ngRNA that comprises a spacer comprising a sequence selected from the group consisting of SEQ ID Nos: 20395-20415 of Table 3D.

In some embodiments, the PEgRNA comprises a spacer comprising the sequence of SEQ Identifier: A332 of Table 3A. In some embodiments, the PEgRNA comprises a spacer comprising a sequence as set forth in SEQ ID NO. 680 of Table 3A. In some embodiments, the PEgRNA comprises a PBS comprising a sequence selected from the group consisting of SEQ Identifiers: B1386-B1394 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4976-C4986 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D765-D782 of Table 3D. In some embodiments, the prime editing composition further comprises a ngRNA that comprises a spacer comprising a sequence selected from the group consisting of SEQ ID Nos: 20398-20415 of Table 3D.

In some embodiments, the PEgRNA comprises a spacer comprising a sequence selected from the group consisting of SEQ Identifiers: A333-A334 of Table 3A. In some embodiments, the PEgRNA comprises a spacer comprising a sequence as set forth in SEQ ID NOs. 681-682 of Table 3A. In some embodiments, the PEgRNA comprises a PBS comprising a sequence selected from the group consisting of SEQ Identifiers: B1395-B1403 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C4987-C5028 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D783-D803 of Table 3D. In some embodiments, the prime editing composition further comprises a ngRNA that comprises a spacer comprising a sequence selected from the group consisting of SEQ ID Nos: 20416-20436 of Table 3D.

In some embodiments, the PEgRNA comprises a spacer comprising a sequence selected from the group consisting of SEQ Identifiers: A371-A373 of Table 3A. In some embodiments, the PEgRNA comprises a spacer comprising a sequence as set forth in SEQ ID NOs. 719-721 of Table 3A. In some embodiments, the PEgRNA comprises a PBS comprising a sequence selected from the group consisting of SEQ Identifiers: B1539-B1547. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5029-C5059 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D797, D802, D803, D805, D806, D809, D810, D894, D896, D904, D905, and D906 of Table 3D. In some embodiments, the prime editing composition further comprises a ngRNA that comprises a spacer comprising a sequence selected from the group consisting of SEQ ID Nos: 20430, 20435-20436, 20438-20439, 20442-20443, 20527, 20529, and 20537-20539 of Table 3D.

In some embodiments, the PEgRNA comprises a spacer comprising the sequence of SEQ Identifier: A374 of Table 3A. In some embodiments, the PEgRNA comprises a spacer comprising a sequence as set forth in SEQ ID NO. 722 of Table 3A. In some embodiments, the PEgRNA comprises a PBS comprising a sequence selected from the group consisting of SEQ Identifier: B1431-B1439 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5060-C5090 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D767-D782, D813, D814, D907, D908, and D909 of Table 3D. In some embodiments, the prime editing composition further comprises a ngRNA that comprises a spacer comprising a sequence selected from the group consisting of SEQ ID Nos: 20400-20415, 20446-20447, and 20540-20542 of Table 3D.

In some embodiments, the PEgRNA comprises a spacer comprising a sequence selected from the group consisting of SEQ Identifiers: A343-A344 of Table 3A. In some embodiments, the PEgRNA comprises a spacer comprising a sequence as set forth in SEQ ID NOs. 691-692 of Table 3A. In some embodiments, the PEgRNA comprises a PBS comprising a sequence selected from the group consisting of SEQ Identifiers: B1440-B1448 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5091-C5140 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D820-D839 of Table 3D. In some embodiments, the prime editing composition further comprises a ngRNA that comprises a spacer comprising a sequence selected from the group consisting of SEQ ID Nos: 20453-20472 of Table 3D.

In some embodiments, the PEgRNA comprises a spacer comprising the sequence of SEQ Identifier: A345 of Table 3A. In some embodiments, the PEgRNA comprises a spacer comprising a sequence as set forth in SEQ ID NO. 693 of Table 3A. In some embodiments, the PEgRNA comprises a PBS comprising a sequence selected from the group consisting of SEQ Identifiers: B1449-B1457 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5141-C5147 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D826-D840 and D899 of Table 3D. In some embodiments, the prime editing composition further comprises a ngRNA that comprises a spacer comprising a sequence selected from the group consisting of SEQ ID Nos: 20459-20473 and 20532 of Table 3D.

In some embodiments, the PEgRNA comprises a spacer comprising the sequence of SEQ Identifier: A375 of Table 3A. In some embodiments, the PEgRNA comprises a spacer comprising a sequence as set forth in SEQ ID NO. 723 of Table 3A. In some embodiments, the PEgRNA comprises a PBS comprising a sequence selected from the group consisting of SEQ Identifiers: B1548-B1556 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising the sequence of SEQ Identifier: C5148 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D826-D840 and D899 of Table 3D. In some embodiments, the prime editing composition further comprises a ngRNA that comprises a spacer comprising a sequence selected from the group consisting of SEQ ID Nos: 20459-20473 and 20532 of Table 3D.

In some embodiments, the PEgRNA comprises a spacer comprising a sequence selected from the group consisting of SEQ Identifiers: A346-A348 of Table 3A. In some embodiments, the PEgRNA comprises a spacer comprising a sequence as set forth in SEQ ID NOs. 694-696 of Table 3A. In some embodiments, the PEgRNA comprises a PBS comprising a sequence selected from the group consisting of SEQ Identifiers: B1458-B1466 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5149-C5157 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D797, D802, D805, D806, D809, D810, D841, D842, and D843 of Table 3D. In some embodiments, the prime editing composition further comprises a ngRNA that comprises a spacer comprising a sequence selected from the group consisting of SEQ ID Nos: 20430, 20435-20436, 20438-20439, 20442-20443, and 20474-20476 of Table 3D.

In some embodiments, the PEgRNA comprises a spacer comprising a sequence selected from the group consisting of SEQ Identifiers: A376-A377 of Table 3A. In some embodiments, the PEgRNA comprises a spacer comprising a sequence as set forth in SEQ ID NOs. 724-725 of Table 3A. In some embodiments, the PEgRNA comprises a PBS comprising a sequence selected from the group consisting of SEQ Identifiers: B1467-B1475 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5158-C5188 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D822-D837, D846-D853, and D910-D913 of Table 3D. In some embodiments, the prime editing composition further comprises a ngRNA that comprises a spacer comprising a sequence selected from the group consisting of SEQ ID Nos: 20455-20470, 20479-20486, and 20543-20546 of Table 3D.

In some embodiments, the PEgRNA comprises a spacer comprising a sequence selected from the group consisting of SEQ Identifiers: A351-A354 of Table 3A. In some embodiments, the PEgRNA comprises a spacer comprising a sequence as set forth in SEQ ID NOs. 699-702 of Table 3A. In some embodiments, the PEgRNA comprises a PBS comprising a sequence selected from the group consisting of SEQ Identifiers: B1476-B1484 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5189-C5236 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D856-D880 of Table 3D. In some embodiments, the prime editing composition further comprises a ngRNA that comprises a spacer comprising a sequence selected from the group consisting of SEQ ID Nos: 20489-20513 of Table 3D.

In some embodiments, the PEgRNA comprises a spacer comprising a sequence selected from the group consisting of SEQ Identifiers: A355-A356 of Table 3A. In some embodiments, the PEgRNA comprises a spacer comprising a sequence as set forth in SEQ ID NOs. 703-704 of Table 3A. In some embodiments, the PEgRNA comprises a PBS comprising a sequence selected from the group consisting of SEQ Identifiers: B1485-B1493 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5237-C5262 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D822-D839, and D846-D853 of Table 3D. In some embodiments, the prime editing composition further comprises a ngRNA that comprises a spacer comprising a sequence selected from the group consisting of SEQ ID Nos: 20455-20472 and 20479-20486 of Table 3D.

In some embodiments, the PEgRNA comprises a spacer comprising a sequence selected from the group consisting of SEQ Identifiers: A378-A379 of Table 3A. In some embodiments, the PEgRNA comprises a spacer comprising a sequence as set forth in SEQ ID NOs. 726-727 of Table 3A. In some embodiments, the PEgRNA comprises a PBS comprising a sequence selected from the group consisting of SEQ Identifiers: B1530-B1538 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5263-C5293 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D763, D764, D768-D782, D813, D814, D818, D907, D908, D909, and D914 of Table 3D. In some embodiments, the prime editing composition further comprises a ngRNA that comprises a spacer comprising a sequence selected from the group consisting of SEQ ID Nos. 20396-20397, 20401-20415, 20446-20447, 20451, and 20540-20542 of Table 3D.

In some embodiments, the PEgRNA comprises a spacer comprising the sequence of SEQ Identifier: A380 of Table 3A. In some embodiments, the PEgRNA comprises a spacer comprising a sequence as set forth in SEQ ID NO. 728 of Table 3A. In some embodiments, the PEgRNA comprises a PBS comprising a sequence selected from the group consisting of SEQ Identifiers: B1557-B1565 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5294-C5324 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D768-D782, D813, D814, D907, D908, and D909 of Table 3D. In some embodiments, the prime editing composition further comprises a ngRNA that comprises a spacer comprising a sequence selected from the group consisting of SEQ ID Nos: 20401-20415, 20446-20447, and 20540-20542 of Table 3D.

In some embodiments, the PEgRNA comprises a spacer comprising a sequence selected from the group consisting of SEQ Identifiers: A357-A358 of Table 3A. In some embodiments, the PEgRNA comprises a spacer comprising a sequence as set forth in SEQ ID NOs. 705-706 of Table 3A. In some embodiments, the PEgRNA comprises a PBS comprising a sequence selected from the group consisting of SEQ Identifiers: B1494-B1502 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5325-C5346 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D765-D782, D813, D814, D881-D889, and D907 of Table 3D. In some embodiments, the prime editing composition further comprises a ngRNA that comprises a spacer comprising a sequence selected from the group consisting of SEQ ID Nos: 20398-20415, 20446-20447, 20514-20522, and 20539 of Table 3D.

In some embodiments, the PEgRNA comprises a spacer comprising a sequence selected from the group consisting of SEQ Identifiers: A359-A360 of Table 3A. In some embodiments, the PEgRNA comprises a spacer comprising a sequence as set forth in SEQ ID NO. 707-708 of Table 3A. In some embodiments, the PEgRNA comprises a PBS comprising a sequence selected from the group consisting of SEQ Identifiers: B1503-B1511 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5347-5408C of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D862-D868, and D872-D879 of Table 3D. In some embodiments, the prime editing composition further comprises a ngRNA that comprises a spacer comprising a sequence selected from the group consisting of SEQ ID Nos: 20495-20501 and 20505-20512 of Table 3D.

In some embodiments, the PEgRNA comprises a spacer comprising the sequence of SEQ Identifier: A361 of Table 3A. In some embodiments, the PEgRNA comprises a spacer comprising a sequence as set forth in SEQ ID NO. 709 of Table 3A. In some embodiments, the PEgRNA comprises a PBS comprising a sequence selected from the group consisting of SEQ Identifiers: B1512-B1520 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5409-C5414 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D765-D781 of Table 3D. In some embodiments, the prime editing composition further comprises a ngRNA that comprises a spacer comprising a sequence selected from the group consisting of SEQ ID Nos: 20398-20414 of Table 3D.

In some embodiments, the editing target sequence is in exon 12 of the CFTR gene.

In some embodiments, the editing target sequence or the complementary sequence on the CFTR gene thereof comprises a mutation that encodes an amino acid substitution G542*, E543A, G544C, G544V, and/or G545V relative to a wild type CFTR polypeptide set forth in SEQ ID NO: 19, wherein * refers to a non-sense mutation and wherein the editing template encodes an intended nucleotide edit that corrects the mutation. In some embodiments, the editing target sequence or the complementary sequence on the CFTR gene thereof comprises a G→→T mutation (c.1624G>T) at position 117,587,778, a duplication of nucleotides TA (c.1616_1617dupTA) at position 117,587,771, a A→→C mutation (c.1628A>C) at position 117,587,782, a G→→T mutation (c.1630G>T) at position 117,587,784, a G→→T mutation (c.1631G>T) at position 117,587,785, and/or a G→→T mutation (c.1634G>T) at position 117,587,788 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide. In some embodiments, the editing template comprises a wild type CFTR gene sequence. Accordingly, in some embodiments, the newly synthesized single stranded DNA encoded by the editing template is capable of correcting the c.1624G>T mutation, the c.1616_1617dupTA mutation, the c.1628A>C mutation, the c.1630G>T mutation, the c.1631G>T mutation, and/or the c.1634G>T mutation in the target CFTR gene. In some embodiments, the newly synthesized single stranded DNA encoded by the editing template is capable of correcting all of the c.1624G>T mutation, the c.1616_1617dupTA mutation, the c.1628A>C mutation, the c.1630G>T mutation, the c.1631G>T mutation, and the c.1634G>T mutation in the target CFTR gene. In some embodiments, two or more of the c.1624G>T mutation, the c.1616_1617dupTA mutation, the c.1628A>C mutation, the c.1630G>T mutation, the c.1631G>T mutation, and the c.1634G>T mutation are located on the same CFTR gene. In some embodiments, the newly synthesized single stranded DNA encoded by the editing template is capable of correcting the c.1624G>T mutation, the c.1616_1617dupTA mutation, the c.1628A>C mutation, the c.1630G>T mutation, the c.1631G>T mutation, and the c.1634G>T mutation that are located on different CFTR genes. For example, in some embodiments, a subject may comprise two copies of CFTR genes, each comprising a different mutation selected from the group consisting of the c.1624G>T mutation, the c.1616_1617dupTA mutation, the c.1628A>C mutation, the c.1630G>T mutation, the c.1631G>T mutation, and the c.1634G>T mutation, all of which may be corrected by an editing template of a single PEgRNA. In some embodiments, the editing template is capable of correcting one or more, or all of the c.1624G>T mutation, the c.1616_1617dupTA mutation, the c.1628A>C mutation, the c.1630G>T mutation, the c.1631G>T mutation, and the c.1634G>T mutation that are in different subjects, wherein each subject may have one or more of the mutations.

In some embodiments, the PEgRNA comprises a spacer comprising a sequence selected from the group consisting of SEQ Identifiers: A381-A382 of Table 3A. In some embodiments, the PEgRNA comprises a spacer comprising a sequence as set forth in SEQ ID NOs. 729-730 of Table 3A. In some embodiments, the PEgRNA comprises a PBS comprising a sequence selected from the group consisting of SEQ Identifiers: B1566-B1574 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5415-C5476 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D915-D928 of Table 3D. In some embodiments, the prime editing composition further comprises a ngRNA that comprises a spacer comprising a sequence selected from the group consisting of SEQ ID Nos: 20548-20561 of Table 3D.

In some embodiments, the PEgRNA comprises a spacer comprising a sequence selected from the group consisting of SEQ Identifiers: A383-A384 of Table 3A. In some embodiments, the PEgRNA comprises a spacer comprising a sequence as set forth in SEQ ID NOs. 731-732 of Table 3A. In some embodiments, the PEgRNA comprises a PBS comprising a sequence selected from the group consisting of SEQ Identifiers: B1575-B1583 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5477-C5507 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D929-D952 of Table 3D. In some embodiments, the prime editing composition further comprises a ngRNA that comprises a spacer comprising a sequence selected from the group consisting of SEQ ID Nos: 20562-20585 of Table 3D.

In some embodiments, the PEgRNA comprises a spacer comprising a sequence selected from the group consisting of SEQ Identifiers: A385-A386 of Table 3A. In some embodiments, the PEgRNA comprises a spacer comprising a sequence as set forth in SEQ ID NOs. 733-734 of Table 3A. In some embodiments, the PEgRNA comprises a PBS comprising a sequence selected from the group consisting of SEQ Identifiers: B1584-B1592 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5508-C5521 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D929-D943, and D946-D953 of Table 3D. In some embodiments, the prime editing composition further comprises a ngRNA that comprises a spacer comprising a sequence selected from the group consisting of SEQ ID Nos: 20562-20576 and 20579-20586 of Table 3D.

In some embodiments, the PEgRNA comprises a spacer comprising a sequence selected from the group consisting of SEQ Identifiers: A387-A388 of Table 3A. In some embodiments, the PEgRNA comprises a spacer comprising a sequence as set forth in SEQ ID NOs. 735-736 of Table 3A. In some embodiments, the PEgRNA comprises a PBS comprising a sequence selected from the group consisting of SEQ Identifiers: B1593-B1601 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5522-C5559 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D915-D928, D954, and D955 of Table 3D. In some embodiments, the prime editing composition further comprises a ngRNA that comprises a spacer comprising a sequence selected from the group consisting of SEQ ID Nos: 20548-20561 and 20587-20588 of Table 3D.

In some embodiments, the PEgRNA comprises a spacer comprising a sequence selected from the group consisting of SEQ Identifiers: A389-A390 of Table 3A. In some embodiments, the PEgRNA comprises a spacer comprising a sequence as set forth in SEQ ID NOs. 737-738 of Table 3A. In some embodiments, the PEgRNA comprises a PBS comprising a sequence selected from the group consisting of SEQ Identifiers: B1602-B1610 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5560-C5580 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D956-D981 of Table 3D. In some embodiments, the prime editing composition further comprises a ngRNA that comprises a spacer comprising a sequence selected from the group consisting of SEQ ID Nos: 2058920614 of Table 3D.

In some embodiments, the PEgRNA comprises a spacer comprising a sequence selected from the group consisting of SEQ Identifiers: A391-A392 of Table 3A. In some embodiments, the PEgRNA comprises a spacer comprising a sequence as set forth in SEQ ID NOs. 739-740 of Table 3A. In some embodiments, the PEgRNA comprises a PBS comprising a sequence selected from the group consisting of SEQ Identifiers: B1611-B1619 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5581-C5611 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D929-D952 of Table 3D. In some embodiments, the prime editing composition further comprises a ngRNA that comprises a spacer comprising a sequence selected from the group consisting of SEQ ID Nos: 20562-20585 of Table 3D.

In some embodiments, the PEgRNA comprises a spacer comprising a sequence selected from the group consisting of SEQ Identifiers: A393-A394 of Table 3A. In some embodiments, the PEgRNA comprises a spacer comprising a sequence as set forth in SEQ ID NOs. 741-742 of Table 3A. In some embodiments, the PEgRNA comprises a PBS comprising a sequence selected from the group consisting of SEQ Identifiers: B1620-B1628 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5612-C5627 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D929-D943 and D946-D953 of Table 3D. In some embodiments, the prime editing composition further comprises a ngRNA that comprises a spacer comprising a sequence selected from the group consisting of SEQ ID Nos: 20562-20576 and 20579-20586 of Table 3D.

In some embodiments, the PEgRNA comprises a spacer comprising the sequence of SEQ Identifier: A395 of Table 3A. In some embodiments, the PEgRNA comprises a spacer comprising a sequence as set forth in SEQ ID NO. 743 of Table 3A. In some embodiments, the PEgRNA comprises a PBS comprising a sequence selected from the group consisting of SEQ Identifiers: B1629-B1637 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5628-C5646 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D963-D981 of Table 3D. In some embodiments, the prime editing composition further comprises a ngRNA that comprises a spacer comprising a sequence selected from the group consisting of SEQ ID Nos: 20596-20614 of Table 3D.

In some embodiments, the PEgRNA comprises a spacer comprising a sequence selected from the group consisting of SEQ Identifiers: A396-A397 of Table 3A. In some embodiments, the PEgRNA comprises a spacer comprising a sequence as set forth in SEQ ID NOs. 744-745 of Table 3A. In some embodiments, the PEgRNA comprises a PBS comprising a sequence selected from the group consisting of SEQ Identifiers: B1638-B1646 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5647-C5676 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D915-D928, D982, and D983 of Table 3D. In some embodiments, the prime editing composition further comprises a ngRNA that comprises a spacer comprising a sequence selected from the group consisting of SEQ ID Nos: 20548-20561 and 20615-20616 of Table 3D.

In some embodiments, the PEgRNA comprises a spacer comprising a sequence selected from the group consisting of SEQ Identifiers: A398-A399 of Table 3A. In some embodiments, the PEgRNA comprises a spacer comprising a sequence as set forth in SEQ ID NOs. 746-747 of Table 3A. In some embodiments, the PEgRNA comprises a PBS comprising a sequence selected from the group consisting of SEQ Identifiers: B1647-B1655 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5677-C5738 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D984-D1001 of Table 3D. In some embodiments, the prime editing composition further comprises a ngRNA that comprises a spacer comprising a sequence selected from the group consisting of SEQ ID Nos: 20617-20634 of Table 3D.

In some embodiments, the PEgRNA comprises a spacer comprising a sequence selected from the group consisting of SEQ Identifiers: A400-A401 of Table 3A. In some embodiments, the PEgRNA comprises a spacer comprising a sequence as set forth in SEQ ID NOs. 748-749 of Table 3A. In some embodiments, the PEgRNA comprises a PBS comprising a sequence selected from the group consisting of SEQ Identifiers: B1656-B1664 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5739-C5743 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D958, D959, D960, D967-D981 and D1002 of Table 3D. In some embodiments, the prime editing composition further comprises a ngRNA that comprises a spacer comprising a sequence selected from the group consisting of SEQ ID Nos 20591-20593, 20600-20614, and 20635 of Table 3D.

In some embodiments, the PEgRNA comprises a spacer comprising the sequence of SEQ Identifiers: A402 of Table 3A. In some embodiments, the PEgRNA comprises a spacer comprising a sequence as set forth in SEQ ID NO. 750 of Table 3A. In some embodiments, the PEgRNA comprises a PBS comprising a sequence selected from the group consisting of SEQ Identifiers: B1665-B1673 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5744-C5754 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D929-D941 and D953 of Table 3D. In some embodiments, the prime editing composition further comprises a ngRNA that comprises a spacer comprising a sequence selected from the group consisting of SEQ ID Nos: 20562-20574 and 20654-20658 of Table 3D.

In some embodiments, the PEgRNA comprises a spacer comprising a sequence selected from the group consisting of SEQ Identifiers: A403-A404 of Table 3A. In some embodiments, the PEgRNA comprises a spacer comprising a sequence as set forth in SEQ ID NOs. 751-752 of Table 3A. In some embodiments, the PEgRNA comprises a PBS comprising a sequence selected from the group consisting of SEQ Identifiers: B1674-B1682 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5755-C5780 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D915-D928, D1003, and D1004 of Table 3D. In some embodiments, the prime editing composition further comprises a ngRNA that comprises a spacer comprising a sequence selected from the group consisting of SEQ ID Nos: 20548-20561 and 20636-20637 of Table 3D.

In some embodiments, the PEgRNA comprises a spacer comprising a sequence selected from the group consisting of SEQ Identifiers: A405-A406 of Table 3A. In some embodiments, the PEgRNA comprises a spacer comprising a sequence as set forth in SEQ ID NOs. 753-754 of Table 3A. In some embodiments, the PEgRNA comprises a PBS comprising a sequence selected from the group consisting of SEQ Identifiers: B1683-B1691 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5781-C5800 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D929-D943, and D946-D953 of Table 3D. In some embodiments, the prime editing composition further comprises a ngRNA that comprises a spacer comprising a sequence selected from the group consisting of SEQ ID Nos: 20562-20576 and 20579-20586 of Table 3D.

In some embodiments, the PEgRNA comprises a spacer comprising a sequence selected from the group consisting of SEQ Identifiers: A407-A408 of Table 3A. In some embodiments, the PEgRNA comprises a spacer comprising a sequence as set forth in SEQ ID NOs. 755-756 of Table 3A. In some embodiments, the PEgRNA comprises a PBS comprising a sequence selected from the group consisting of SEQ Identifiers: B1692-B1700 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5801-C5831 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D956-D980, and D1005-D1011 of Table 3D. In some embodiments, the prime editing composition further comprises a ngRNA that comprises a spacer comprising a sequence selected from the group consisting of SEQ ID Nos: 20589-20613 and 20638-20644 of Table 3D.

In some embodiments, the PEgRNA comprises a spacer comprising the sequence of SEQ Identifier: A409 of Table 3A. In some embodiments, the PEgRNA comprises a spacer comprising a sequence as set forth in SEQ ID NO. 757 of Table 3A. In some embodiments, the PEgRNA comprises a PBS comprising a sequence selected from the group consisting of SEQ Identifiers: B1701-B1709 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5832-C5833 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D968-D981, and D1002 of Table 3D. In some embodiments, the prime editing composition further comprises a ngRNA that comprises a spacer comprising a sequence selected from the group consisting of SEQ ID Nos 20601-20614 and 20635 of Table 3D.

In some embodiments, the PEgRNA comprises a spacer comprising a sequence selected from the group consisting of SEQ Identifiers: A410-A411 of Table 3A. In some embodiments, the PEgRNA comprises a spacer comprising a sequence as set forth in SEQ ID NOs. 758-759 of Table 3A. In some embodiments, the PEgRNA comprises a PBS comprising a sequence selected from the group consisting of SEQ Identifiers: B1710-B1718 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5834-C5865 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D986-D992, D995-D1001, and D1012-D1015 of Table 3D. In some embodiments, the prime editing composition further comprises a ngRNA that comprises a spacer comprising a sequence selected from the group consisting of SEQ ID Nos: 20619-20625, 20628-20634, and 20645-20648 of Table 3D.

In some embodiments, the PEgRNA comprises a spacer comprising the sequence of SEQ Identifiers: A412 of Table 3A. In some embodiments, the PEgRNA comprises a spacer comprising a sequence as set forth in SEQ ID NO. 760 of Table 3A. In some embodiments, the PEgRNA comprises a PBS comprising a sequence selected from the group consisting of SEQ Identifiers: B1719-B1727 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5866-C5882 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D964-D981 and D1002 of Table 3D. In some embodiments, the prime editing composition further comprises a ngRNA that comprises a spacer comprising a sequence selected from the group consisting of SEQ ID Nos: 20597-20614 and 20635 of Table 3D.

In some embodiments, the PEgRNA comprises a spacer comprising a sequence selected from the group consisting of SEQ Identifiers: A413-A414 of Table 3A. In some embodiments, the PEgRNA comprises a spacer comprising a sequence as set forth in SEQ ID NOs. 761-762 of Table 3A. In some embodiments, the PEgRNA comprises a PBS comprising a sequence selected from the group consisting of SEQ Identifiers: B1728-B1736 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5883-C5894 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D915-D920, and D922-D927 of Table 3D. In some embodiments, the prime editing composition further comprises a ngRNA that comprises a spacer comprising a sequence selected from the group consisting of SEQ ID Nos: 20548-20553 and 20555-20560 of Table 3D.

In some embodiments, the PEgRNA comprises a spacer comprising the sequence of SEQ Identifier: A415 of Table 3A. In some embodiments, the PEgRNA comprises a spacer comprising a sequence as set forth in SEQ ID NO. 763 of Table 3A. In some embodiments, the PEgRNA comprises a PBS comprising a sequence selected from the group consisting of SEQ Identifiers: B1737-B1745 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5895-C5901 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D929-D939 and D953 of Table 3D. In some embodiments, the prime editing composition further comprises a ngRNA that comprises a spacer comprising a sequence selected from the group consisting of SEQ ID Nos: 20562-20572 and 20586 of Table 3D.

In some embodiments, the PEgRNA comprises a spacer comprising a sequence selected from the group consisting of SEQ Identifiers: A416-A417 of Table 3A. In some embodiments, the PEgRNA comprises a spacer comprising a sequence as set forth in SEQ ID NO. 764-765 of Table 3A. In some embodiments, the PEgRNA comprises a PBS comprising a sequence selected from the group consisting of SEQ Identifiers: B1746-B1754 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5902-C5957 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D915-D928 of Table 3D. In some embodiments, the prime editing composition further comprises a ngRNA that comprises a spacer comprising a sequence selected from the group consisting of SEQ ID Nos. 20548-20561 of Table 3D.

In some embodiments, the PEgRNA comprises a spacer comprising the sequence of SEQ Identifier: A418 of Table 3A. In some embodiments, the PEgRNA comprises a spacer comprising a sequence as set forth in SEQ ID NO. 766 of Table 3A. In some embodiments, the PEgRNA comprises a PBS comprising a sequence selected from the group consisting of SEQ Identifiers: B1755-B1763 of Table 3B. In some embodiments, the PEgRNA comprises a PBS comprising a sequence selected from the group consisting of SEQ ID Nos: of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C5958-C5986 of Table 3C. In some embodiments, the PEgRNA comprises an editing template comprising a sequence selected from the group consisting of SEQ ID Nos: of Table 3C. In some embodiments, the prime editing composition further comprises a ngRNA that comprises a spacer comprising a sequence selected from the group consisting of SEQ Identifiers: D929-D944 of Table 3D. In some embodiments, the prime editing composition further comprises a ngRNA that comprises a spacer comprising a sequence selected from the group consisting of SEQ ID Nos: 20562-20577 of Table 3D.

In some embodiments, the editing target sequence or the complementary sequence on the CFTR gene thereof comprises a mutation that encodes an amino acid substitution L548Q, S549I, S549R, G550*, G551A, G551S, G551D, Q552K, R553G, R553*, and/or A554E relative to a wild type CFTR polypeptide set forth in SEQ ID NO: 19, wherein * refers to a non-sense mutation, and wherein the editing template encodes an intended nucleotide edit that corrects the mutation. In some embodiments, the editing target sequence or the complementary sequence on the CFTR gene thereof comprises a A→→C mutation (c.1645A>C) at position 117,587,799, a deletion of nucleotides ATCACA (c.1636_1641delATCACA) at position 117,587,790, a deletion of nucleotides CT (c.1642_1643delCT) at position 117,587,796, a T→→A mutation (c.1643T>A) at position 117,587,797, a G→→T mutation (c.1646G>T) at position 117,587,800, a T→→G mutation (c.1647T>G) at position 117,587,801, a G→→T mutation (c.1648G>T) at position 117,587,802, a deletion of A (c.1650delA) at position 117,587,804, a G→→A mutation (c.1651G>A) at position 117,587,805, a G→→C mutation (c.1652G>C) at position 117,587,806, G→→A mutation (c.1652G>A) at position 117,587,806, a deletion of G (c.1652delG) at position 117,587,806, a C→→A mutation (c.1654C>A) at position 117,587,808, a deletion of A (c.1656delA) at position 117,587,810, a C→→G mutation (c.1657C>G) at position 117,587,811, a C→→A mutation (c.1661C>A) at position 117,587,815, a C→→G mutation (c.1657C>G) at position 117,587,811, a C→→A mutation (c.1654C>A) at position 117,587,808, a deletion of A (c.1656delA) at position 117,587,810, a C→→G mutation (c.1657C>G) at position 117,587,811, a C→→T mutation (c.1657C>T) at position 117,587,811, an insertion of A (c.1660_1661insA) at position 117,587,814, and/or a C→→A mutation (c.1661C>A) at position 117,587,815 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, the editing template comprises a wild type CFTR gene sequence. Accordingly, in some embodiments, the newly synthesized single stranded DNA encoded by the editing template is capable of correcting the c.1645A>C mutation, the c.1636_1641delATCACA mutation, the c.1642_1643delCT mutation, the c.1643T>A mutation, the c.1646G>T mutation, the c.1647T>G mutation, the c.1648G>T mutation, the c.1650delA mutation, the c.1652G>C mutation, the c.1651G>A mutation, the c.1652G>A mutation, the c.1652delG mutation, the c.1656delA mutation, the c.1657C>G mutation, the c.1661C>A mutation, the c.1657C>G mutation, the c.1654C>A mutation, the c.1657C>T mutation, the c.1660_1661insA mutation, and/or the c.1654C>A mutation in the target CFTR gene. In some embodiments, the newly synthesized single stranded DNA encoded by the editing template is capable of correcting all of the c.1645A>C mutation, the c.1636_1641delATCACA mutation, the c.1642_1643delCT mutation, the c.1643T>A mutation, the c.1646G>T mutation, the c.1647T>G mutation, the c.1648G>T mutation, the c.1650delA mutation, the c.1652G>C mutation, the c.1652delG mutation, the c.1652G>A mutation, the c.1656delA mutation, the c.1651G>A mutation, the c.1656delA mutation, the c.1657C>G mutation, the c.1661C>A mutation, the c.1657C>G mutation, the c.1654C>A mutation, the c.1657C>T mutation, the c.1660_1661insA mutation, and the c.1654C>A mutation in the target CFTR gene. In some embodiments, two or more of the c.1645A>C mutation, the c.1636_1641delATCACA mutation, the c.1642_1643delCT mutation, the c.1643T>A mutation, the c.1646G>T mutation, the c.1647T>G mutation, the c.1648G>T mutation, the c.1650delA mutation, the c.1652G>C mutation, the c.1652delG mutation, the c.1652G>A mutation, the c.1651G>A mutation, the c.1656delA mutation, the c.1656delA mutation, the c.1657C>G mutation, the c.1661C>A mutation, the c.1657C>G mutation, the c.1654C>A mutation, the c.1657C>T mutation, the c.1660_1661insA mutation, and the c.1654C>A mutation are located on the same CFTR gene. In some embodiments, the newly synthesized single stranded DNA encoded by the editing template is capable of correcting the c.1645A>C mutation, the c.1636_1641delATCACA mutation, the c.1642_1643delCT mutation, the c.1643T>A mutation, the c.1646G>T mutation, the c.1647T>G mutation, the c.1648G>T mutation, the c.1650delA mutation, the c.1652G>C mutation, the c.1652delG mutation, the c.1651G>A mutation, the c.1652G>A mutation, the c.1656delA mutation, and the c.1654C>A mutation that are located on different CFTR genes. For example, in some embodiments, a subject may comprise two copies of CFTR genes, each comprising a different mutation selected from the group consisting of the c.1645A>C mutation, the c.1636_1641delATCACA mutation, the c.1642_1643delCT mutation, the c.1643T>A mutation, the c.1646G>T mutation, the c.1647T>G mutation, the c.1648G>T mutation, the c.1650delA mutation, the c.1652G>A mutation, the c.1652G>C mutation, the c.1652delG mutation, the c.1651G>A mutation, the c.1656delA mutation, the c.1656delA mutation, the c.1657C>G mutation, the c.1661C>A mutation, the c.1657C>G mutation, the c.1654C>A mutation, the c.1657C>T mutation, the c.1660_1661insA mutation, and the c.1654C>A mutation, all of which may be corrected by an editing template of a single PEgRNA. In some embodiments, the editing template is capable of correcting one or more, or all of the c.1645A>C mutation, the c.1636_1641delATCACA mutation, the c.1642_1643delCT mutation, the c.1643T>A mutation, the c.1646G>T mutation, the c.1647T>G mutation, the c.1648G>T mutation, the c.1650delA mutation, the c.1652G>C mutation, the c.1652delG mutation, the c.1651G>A mutation, the c.1652G>A mutation, the c.1656delA mutation, the c.1656delA mutation, the c.1657C>G mutation, the c.1661C>A mutation, the c.1657C>G mutation, the c.1654C>A mutation, the c.1657C>T mutation, the c.1660_1661insA mutation, and the c.1654C>A mutation that are in different subjects, wherein each subject may have one or more of the mutations.

In some embodiments, the PEgRNA comprises a spacer comprising a sequence selected from the group consisting of SEQ Identifiers: A419-A420 of Table 3A. In some embodiments, the PEgRNA comprises a spacer comprising a sequence as set forth in SEQ ID NOs. 767-768 of Table 3A. In some embodiments, the PEgRNA comprises a PBS comprising a sequence selected from the group consisting of SEQ Identifiers: B1566-B1574 of Table 3B. In some embodiments, the PEgRNA comprises a PBS comprising a sequence selected from the group consisting of SEQ ID Nos: 2431-2437 of Table 3B. In some embodiments, the PEgRNA comprises a PBS comprising a sequence selected from the group consisting of sequences GAACTATA and GAACTATAT as disclosed in Table 3B. In some embodiments, the PEgRNA comprises an editing template comprising a sequence selected from the group consisting of SEQ Identifiers: C6627-C6656 of Table 3C. In some embodiments, the PEgRNA comprises an editing template comprising a sequence selected from the group consisting of SEQ ID Nos: 10653-10682 of Table 3C. In some embodiments, the prime editing composition further comprises a ngRNA that comprises a spacer comprising a sequence selected from the group consisting of SEQ Identifiers: D915-D928 of Table 3D. In some embodiments, the prime editing composition further comprises a ngRNA that comprises a spacer comprising a sequence selected from the group consisting of SEQ ID Nos: 20548-20561 of Table 3D.

In some embodiments, the PEgRNA comprises a spacer comprising a sequence selected from the group consisting of SEQ Identifiers: A421-A422 of Table 3A. In some embodiments, the PEgRNA comprises a spacer comprising a sequence as set forth in SEQ ID NOs. 469-470 of Table 3A. In some embodiments, the PEgRNA comprises a PBS comprising a sequence selected from the group consisting of SEQ Identifiers: B1575-B1583 of Table 3B. In some embodiments, the PEgRNA comprises a PBS comprising a sequence selected from the group consisting of SEQ ID Nos: 2438-2444 of Table 3B. In some embodiments, the PEgRNA comprises a PBS comprising a sequence selected from the group consisting of sequences AGAACTAT and AGAACTATA as disclosed in Table 3B. In some embodiments, the PEgRNA comprises an editing template comprising a sequence selected from the group consisting of SEQ Identifiers: C6657-C6672 of Table 3C. In some embodiments, the PEgRNA comprises an editing template comprising a sequence selected from the group consisting of SEQ ID Nos: 10683-10698 of Table 3C. In some embodiments, the prime editing composition further comprises a ngRNA that comprises a spacer comprising a sequence selected from the group consisting of SEQ Identifiers: D929-D944, D946-D952, and D1016 of Table 3D. In some embodiments, the prime editing composition further comprises a ngRNA that comprises a spacer comprising a sequence selected from the group consisting of SEQ ID Nos: 20552-20577, 20579-20585, and 20649 of Table 3D.

In some embodiments, the PEgRNA comprises a spacer comprising a sequence selected from the group consisting of SEQ Identifiers: A463-A464 of Table 3A. In some embodiments, the PEgRNA comprises a spacer comprising a sequence as set forth in SEQ ID NOs. 811-812 of Table 3A. In some embodiments, the PEgRNA comprises a PBS comprising a sequence selected from the group consisting of SEQ Identifiers: B1764-B1772 of Table 3B. In some embodiments, the PEgRNA comprises a PBS comprising a sequence selected from the group consisting of SEQ ID Nos: 2585-2591 of Table 3B. In some embodiments, the PEgRNA comprises a PBS comprising a sequence selected from the group consisting of sequences AGTGTGAT and AGTGTGATT as disclosed in Table 3B. In some embodiments, the PEgRNA comprises an editing template comprising a sequence selected from the group consisting of SEQ Identifiers: C6673-C6703 of Table 3C. In some embodiments, the PEgRNA comprises an editing template comprising a sequence selected from the group consisting of SEQ ID Nos: 10699-10729 of Table 3C. In some embodiments, the prime editing composition further comprises a ngRNA that comprises a spacer comprising a sequence selected from the group consisting of SEQ Identifiers: D930-D944, D1016-D1019, and D1053 of Table 3D. In some embodiments, the prime editing composition further comprises a ngRNA that comprises a spacer comprising a sequence selected from the group consisting of SEQ ID Nos: 20553-20577, 20649-20652, and 20686 of Table 3D.

In some embodiments, the PEgRNA comprises a spacer comprising a sequence selected from the group consisting of SEQ Identifiers: A425-A426 of Table 3A. In some embodiments, the PEgRNA comprises a spacer comprising a sequence as set forth in SEQ ID NOs. 773-774 of Table 3A. In some embodiments, the PEgRNA comprises a PBS comprising a sequence selected from the group consisting of SEQ Identifiers: B1773-B1781 of Table 3B. In some embodiments, the PEgRNA comprises a PBS comprising a sequence selected from the group consisting of SEQ ID Nos: 2592-2598 of Table 3B. In some embodiments, the PEgRNA comprises a PBS comprising a sequence selected from the group consisting of sequences CCAAGAAC and CCAAGAACT as disclosed in Table 3B. In some embodiments, the PEgRNA comprises an editing template comprising a sequence selected from the group consisting of SEQ Identifiers: C6704-C6722 of Table 3C. In some embodiments, the PEgRNA comprises an editing template comprising a sequence selected from the group consisting of SEQ ID Nos: 10730-10748 of Table 3C. In some embodiments, the prime editing composition further comprises a ngRNA that comprises a spacer comprising a sequence selected from the group consisting of SEQ Identifiers: D929-D944, and D1016-D1018 of Table 3D. In some embodiments, the prime editing composition further comprises a ngRNA that comprises a spacer comprising a sequence selected from the group consisting of SEQ ID Nos: 20562-20577 and 20649-20651 of Table 3D.

In some embodiments, the PEgRNA comprises a spacer comprising the sequence of SEQ Identifier: A427 of Table 3A. In some embodiments, the PEgRNA comprises a spacer comprising a sequence as set forth in SEQ ID NO. 775 of Table 3A. In some embodiments, the PEgRNA comprises a PBS comprising a sequence selected from the group consisting of SEQ Identifiers: B1782-B1790 of Table 3B. In some embodiments, the PEgRNA comprises a PBS comprising a sequence selected from the group consisting of SEQ ID Nos: 2599-2605 of Table 3B. In some embodiments, the PEgRNA comprises a PBS comprising a sequence selected from the group consisting of sequences TCCAAGAA and TCCAAGAAC as disclosed in Table 3B. In some embodiments, the PEgRNA comprises an editing template comprising a sequence selected from the group consisting of SEQ Identifiers: C6723-C6742 of Table 3C. In some embodiments, the PEgRNA comprises an editing template comprising a sequence selected from the group consisting of SEQ ID Nos: 10749-10768 of Table 3C. In some embodiments, the prime editing composition further comprises a ngRNA that comprises a spacer comprising a sequence selected from the group consisting of SEQ Identifiers: D929-D944, D1016, and D1019 of Table 3D. In some embodiments, the prime editing composition further comprises a ngRNA that comprises a spacer comprising a sequence selected from the group consisting of SEQ ID Nos: 20562-20577, 20649 and 20652 of Table 3D.

In some embodiments, the PEgRNA comprises a spacer comprising a sequence selected from the group consisting of SEQ Identifiers: A428-A429 of Table 3A. In some embodiments, the PEgRNA comprises a spacer comprising a sequence as set forth in SEQ ID NOs. 776-779 of Table 3A. In some embodiments, the PEgRNA comprises a PBS comprising a sequence selected from the group consisting of SEQ Identifiers: B1791-B1799 of Table 3B. In some embodiments, the PEgRNA comprises a PBS comprising a sequence selected from the group consisting of SEQ ID Nos: 2606-2612 of Table 3B. In some embodiments, the PEgRNA comprises a PBS comprising a sequence selected from the group consisting of sequences ACTATATT and ACTATATTG as disclosed in Table 3B. In some embodiments, the PEgRNA comprises an editing template comprising a sequence selected from the group consisting of SEQ Identifiers: C6743-C6755 of Table 3C. In some embodiments, the PEgRNA comprises an editing template comprising a sequence selected from the group consisting of SEQ ID Nos: 10769-10781 of Table 3C. In some embodiments, the prime editing composition further comprises a ngRNA that comprises a spacer comprising a sequence selected from the group consisting of SEQ Identifiers: D929-D944 and D1016-D1018 of Table 3D. In some embodiments, the prime editing composition further comprises a ngRNA that comprises a spacer comprising a sequence selected from the group consisting of SEQ ID Nos: 20562-20577 and 20649-20651 of Table 3D.

In some embodiments, the PEgRNA comprises a spacer comprising a sequence selected from the group consisting of SEQ Identifiers: A465-A467 of Table 3A. In some embodiments, the PEgRNA comprises a spacer comprising a sequence as set forth in SEQ ID NOs. 813-815 of Table 3A. In some embodiments, the PEgRNA comprises a PBS comprising a sequence selected from the group consisting of SEQ Identifiers: B1800-B1808 of Table 3B. In some embodiments, the PEgRNA comprises a PBS comprising a sequence selected from the group consisting of SEQ ID Nos: 2613-2619 of Table 3B. In some embodiments, the PEgRNA comprises a PBS comprising a sequence selected from the group consisting of sequences GTGTGATT and GTGTGATTC as disclosed in Table 3B. In some embodiments, the PEgRNA comprises an editing template comprising a sequence selected from the group consisting of SEQ Identifiers: C6756-C6786 of Table 3C. In some embodiments, the PEgRNA comprises an editing template comprising a sequence selected from the group consisting of SEQ ID Nos: 10782-10812 of Table 3C. In some embodiments, the prime editing composition further comprises a ngRNA that comprises a spacer comprising a sequence selected from the group consisting of SEQ Identifiers: D922, D1021, D1022, D1023, D1024, and D1025 of Table 3D. In some embodiments, the prime editing composition further comprises a ngRNA that comprises a spacer comprising a sequence selected from the group consisting of SEQ ID Nos: 20555 and 20654-20658 of Table 3D.

In some embodiments, the PEgRNA comprises a spacer comprising a sequence selected from the group consisting of SEQ Identifiers: A433-A435 of Table 3A. In some embodiments, the PEgRNA comprises a spacer comprising a sequence as set forth in SEQ ID NOs. 781-783 of Table 3A. In some embodiments, the PEgRNA comprises a PBS comprising a sequence selected from the group consisting of SEQ Identifiers: B1809-B1817 of Table 3B. In some embodiments, the PEgRNA comprises a PBS comprising a sequence selected from the group consisting of SEQ ID Nos: 2620-2626 of Table 3B. In some embodiments, the PEgRNA comprises a PBS comprising a sequence selected from the group consisting of sequences CTATATTG and CTATATTGT as disclosed in Table 3B. In some embodiments, the PEgRNA comprises an editing template comprising a sequence selected from the group consisting of SEQ Identifiers: C6787-C6798 of Table 3C. In some embodiments, the PEgRNA comprises an editing template comprising a sequence selected from the group consisting of SEQ ID Nos: 10813-10824 of Table 3C. In some embodiments, the prime editing composition further comprises a ngRNA that comprises a spacer comprising a sequence selected from the group consisting of SEQ Identifiers: D922, D1021, D1022, D1023, D1024, and D1025 of Table 3D. In some embodiments, the prime editing composition further comprises a ngRNA that comprises a spacer comprising a sequence selected from the group consisting of SEQ ID Nos: 20555 and 20654-20658 of Table 3D.

In some embodiments, the PEgRNA comprises a spacer comprising a sequence selected from the group consisting of SEQ Identifiers: A468-A469 of Table 3A. In some embodiments, the PEgRNA comprises a spacer comprising a sequence as set forth in SEQ ID NOs. 816-817 of Table 3A. In some embodiments, the PEgRNA comprises a PBS comprising a sequence selected from the group consisting of SEQ Identifiers: B1818-B1826 of Table 3B. In some embodiments, the PEgRNA comprises a PBS comprising a sequence selected from the group consisting of SEQ ID Nos: 2627-2633 of Table 3B. In some embodiments, the PEgRNA comprises a PBS comprising a sequence selected from the group consisting of sequences CAGTGTGA and CAGTGTGAT as disclosed in Table 3B. In some embodiments, the PEgRNA comprises an editing template comprising a sequence selected from the group consisting of SEQ Identifiers: C6799-C6829 of Table 3C. In some embodiments, the PEgRNA comprises an editing template comprising a sequence selected from the group consisting of SEQ ID Nos: 10825-10855 of Table 3C. In some embodiments, the prime editing composition further comprises a ngRNA that comprises a spacer comprising a sequence selected from the group consisting of SEQ Identifiers: D930-D944, D946-D952, D1016, D1019, D1026, D1053, and D1054 of Table 3D. In some embodiments, the prime editing composition further comprises a ngRNA that comprises a spacer comprising a sequence selected from the group consisting of SEQ ID Nos: 20563-20577, 20579-20585, 20649, 20652, 20659, and 20686-20687 of Table 3D.

In some embodiments, the PEgRNA comprises a spacer comprising a sequence selected from the group consisting of SEQ Identifiers: A438-A441 of Table 3A. In some embodiments, the PEgRNA comprises a spacer comprising a sequence as set forth in SEQ ID NOs. 786-789 of Table 3A. In some embodiments, the PEgRNA comprises a PBS comprising a sequence selected from the group consisting of SEQ Identifiers: B1827-B1835 of Table 3B. In some embodiments, the PEgRNA comprises a PBS comprising a sequence selected from the group consisting of SEQ ID Nos: 2634-2640 of Table 3B. In some embodiments, the PEgRNA comprises a PBS comprising a sequence selected from the group consisting of sequences TCTCCAAG and TCTCCAAGA as disclosed in Table 3B. In some embodiments, the PEgRNA comprises an editing template comprising a sequence selected from the group consisting of SEQ Identifiers: C6830-C6851 of Table 3C. In some embodiments, the PEgRNA comprises an editing template comprising a sequence selected from the group consisting of SEQ ID Nos: 10856-10877 of Table 3C. In some embodiments, the prime editing composition further comprises a ngRNA that comprises a spacer comprising a sequence selected from the group consisting of SEQ Identifiers: D925, D929-D944, D1016-D1019, and D1028-D1032 of Table 3D. In some embodiments, the prime editing composition further comprises a ngRNA that comprises a spacer comprising a sequence selected from the group consisting of SEQ ID Nos: 20558, 20562-20577, 20649-20652, and 20661-20665 of Table 3D.

In some embodiments, the PEgRNA comprises a spacer comprising the sequence of SEQ Identifier: A442 of Table 3A. In some embodiments, the PEgRNA comprises a spacer comprising a sequence as set forth in SEQ ID NO. 790 of Table 3A. In some embodiments, the PEgRNA comprises a PBS comprising a sequence selected from the group consisting of SEQ Identifiers: B1836-B1844 of Table 3B. In some embodiments, the PEgRNA comprises a PBS comprising a sequence selected from the group consisting of SEQ ID Nos: 2641-2647 of Table 3B. In some embodiments, the PEgRNA comprises a PBS comprising a sequence selected from the group consisting of sequences TGAATAAC and TGAATAACT as disclosed in Table 3B. In some embodiments, the PEgRNA comprises an editing template comprising a sequence selected from the group consisting of SEQ Identifiers: C6852-C6856 of Table 3C. In some embodiments, the PEgRNA comprises an editing template comprising a sequence selected from the group consisting of SEQ ID Nos: 10878-10882 of Table 3C. In some embodiments, the prime editing composition further comprises a ngRNA that comprises a spacer comprising a sequence selected from the group consisting of SEQ Identifiers: D972-D981, D1002, D1033-D1037 and D1055 of Table 3D. In some embodiments, the prime editing composition further comprises a ngRNA that comprises a spacer comprising a sequence selected from the group consisting of SEQ ID Nos: 20605-20614, 20635, 20666-20670 and 20688 of Table 3D.

In some embodiments, the PEgRNA comprises a spacer comprising a sequence selected from the group consisting of SEQ Identifiers: A443-A445 of Table 3A. In some embodiments, the PEgRNA comprises a spacer comprising a sequence selected from the group consisting of SEQ ID NOs. 791-793 of Table 3A. In some embodiments, the PEgRNA comprises a PBS comprising a sequence selected from the group consisting of SEQ Identifiers: B1845-B1853 of Table 3B. In some embodiments, the PEgRNA comprises a PBS comprising a sequence selected from the group consisting of SEQ ID Nos: 2648-2654 of Table 3B. In some embodiments, the PEgRNA comprises a PBS comprising a sequence selected from the group consisting of sequences CTCCAAGA and CTCCAAGAA as disclosed in Table 3B. In some embodiments, the PEgRNA comprises an editing template comprising a sequence selected from the group consisting of SEQ Identifiers: C6857-C6877 of Table 3C. In some embodiments, the PEgRNA comprises an editing template comprising a sequence selected from the group consisting of SEQ ID Nos: 10883-10903 of Table 3C. In some embodiments, the prime editing composition further comprises a ngRNA that comprises a spacer comprising a sequence selected from the group consisting of SEQ Identifiers: D922 and D1021-D1025 of Table 3D. In some embodiments, the prime editing composition further comprises a ngRNA that comprises a spacer comprising a sequence selected from the group consisting of SEQ ID Nos: 20555 and 20654-20658 of Table 3D.

In some embodiments, the PEgRNA comprises a spacer comprising the sequence of SEQ Identifiers: A446 of Table 3A. In some embodiments, the PEgRNA comprises a spacer comprising a sequence as set forth in SEQ ID NO: 791. In some embodiments, the PEgRNA comprises a PBS comprising a sequence selected from the group consisting of SEQ Identifiers: B1854-B1862 of Table 3B. In some embodiments, the PEgRNA comprises a PBS comprising a sequence selected from the group consisting of SEQ ID NOs. 2655-2661 of Table 3B. In some embodiments, the PEgRNA comprises a PBS comprising a sequence selected from the group consisting of sequences ATTTCTTT and ATTTCTTTA, as disclosed in Table 3B. In some embodiments, the PEgRNA comprises an editing template comprising a sequence selected from the group consisting of SEQ Identifiers: C6878-C6897 of Table 3C. In some embodiments, the PEgRNA comprises an editing template comprising a sequence selected from the group consisting of SEQ ID NOs: 10904-10923 of Table 3C. In some embodiments, the prime editing composition further comprises a ngRNA that comprises a spacer comprising a sequence selected from the group consisting of SEQ Identifiers: D968-D981, D1002, D1033, D1034, and D1035 of Table 3D. In some embodiments, the prime editing composition further comprises a ngRNA that comprises a spacer comprising a sequence selected from the group consisting of SEQ ID NOs. 20601-20614, 20635, 20666, 20667, and 20668 of Table 3D.

In some embodiments, the PEgRNA comprises a spacer comprising a sequence selected from the group consisting of SEQ Identifiers: A391-A392 of Table 3A. In some embodiments, the PEgRNA comprises a spacer comprising a sequence as set forth in SEQ ID NOs. 739 and 740 of Table 3A. In some embodiments, the PEgRNA comprises a PBS comprising a sequence selected from the group consisting of SEQ Identifiers: B1611-B1619 of Table 3B. In some embodiments, the PEgRNA comprises a PBS comprising a sequence selected from the group consisting of SEQ ID NOs. 2466-2472 of Table 3B. In some embodiments, the PEgRNA comprises a PBS comprising a sequence selected from the group consisting of sequences AACTATAT and AACTATATT as disclosed in Table 3B. In some embodiments, the PEgRNA comprises an editing template comprising a sequence selected from the group consisting of SEQ Identifiers: C6898-C6911 of Table 3C. In some embodiments, the PEgRNA comprises an editing template comprising a sequence selected from the group consisting of SEQ ID NOs: 10924-10937 of Table 3C. In some embodiments, the prime editing composition further comprises a ngRNA that comprises a spacer comprising a sequence selected from the group consisting of SEQ Identifiers: D929-D944, D946-D952, and D1016 of Table 3D. In some embodiments, the prime editing composition further comprises a ngRNA that comprises a spacer comprising a sequence selected from the group consisting of SEQ ID NOs. 20562-20577, 20579-20585, and 20649 of Table 3D.

In some embodiments, the PEgRNA comprises a spacer comprising a sequence selected from the group consisting of SEQ Identifiers: A447-A450 of Table 3A. In some embodiments, the PEgRNA comprises a spacer comprising a sequence as set forth in SEQ ID NOs. 795-798 of Table 3A. In some embodiments, the PEgRNA comprises a PBS comprising a sequence selected from the group consisting of SEQ Identifiers: B1863-B1871 of Table 3B. In some embodiments, the PEgRNA comprises a PBS comprising a sequence selected from the group consisting of SEQ ID NOs. 2662-2668 of Table 3B. In some embodiments, the PEgRNA comprises a PBS comprising a sequence selected from the group consisting of sequences CAAGAACT and CAAGAACTA as disclosed in Table 3B. In some embodiments, the PEgRNA comprises an editing template comprising a sequence selected from the group consisting of SEQ Identifiers: C6912-C6947 of Table 3C. In some embodiments, the PEgRNA comprises an editing template comprising a sequence selected from the group consisting of SEQ ID NOs: 10938-10973 of Table 3C. In some embodiments, the prime editing composition further comprises a ngRNA that comprises a spacer comprising a sequence selected from the group consisting of SEQ Identifiers: D915-D928, and D1021-D 1025 of Table 3D. In some embodiments, the prime editing composition further comprises a ngRNA that comprises a spacer comprising a sequence selected from the group consisting of SEQ ID NOs. 20548-20561, and 20654-20658 of Table 3D.

In some embodiments, the PEgRNA comprises a spacer comprising a sequence selected from the group consisting of SEQ Identifiers: A456 and A470 of Table 3A. In some embodiments, the PEgRNA comprises a spacer comprising a sequence as set forth in SEQ ID NOs. 804 and 818 of Table 3A. In some embodiments, the PEgRNA comprises a PBS comprising a sequence selected from the group consisting of SEQ Identifiers: B1899-B1907 of Table 3B. In some embodiments, the PEgRNA comprises a PBS comprising a sequence selected from the group consisting of SEQ ID NOs. 2690-2696 of Table 3B. In some embodiments, the PEgRNA comprises a PBS comprising a sequence selected from the group consisting of sequences TGATTCCA and TGATTCCAC as disclosed in Table 3B. In some embodiments, the PEgRNA comprises an editing template comprising a sequence selected from the group consisting of SEQ Identifiers: C6948-C6978 of Table 3C. In some embodiments, the PEgRNA comprises an editing template comprising a sequence selected from the group consisting of SEQ ID NOs: 10974-11004 of Table 3C. In some embodiments, the prime editing composition further comprises a ngRNA that comprises a spacer comprising a sequence selected from the group consisting of SEQ Identifiers: D930-D944, D946-D952, D1016, D1019, and D1026 of Table 3D. In some embodiments, the prime editing composition further comprises a ngRNA that comprises a spacer comprising a sequence selected from the group consisting of SEQ ID NOs. 20563-20577, 20579-20585, 20649, 20652, and 20659 of Table 3D.

In some embodiments, the PEgRNA comprises a spacer comprising a sequence selected from the group consisting of SEQ Identifiers: A471-A474 of Table 3A. In some embodiments, the PEgRNA comprises a spacer comprising a sequence as set forth in SEQ ID NOs. 819-822 of Table 3A In some embodiments, the PEgRNA comprises a PBS comprising a sequence selected from the group consisting of SEQ Identifiers: B1926-B1934 of Table 3B. In some embodiments, the PEgRNA comprises a PBS comprising a sequence selected from the group consisting of SEQ ID NOs. 2711-2717 of Table 3B. In some embodiments, the PEgRNA comprises a PBS comprising a sequence selected from the group consisting of sequences TCAGTGTG and TCAGTGTGA as disclosed in Table 3B. In some embodiments, the PEgRNA comprises an editing template comprising a sequence selected from the group consisting of SEQ Identifiers: C6979-C7040 of Table 3C. In some embodiments, the PEgRNA comprises an editing template comprising a sequence selected from the group consisting of SEQ ID NOs: 11005-11066 of Table 3C. In some embodiments, the prime editing composition further comprises a ngRNA that comprises a spacer comprising a sequence selected from the group consisting of SEQ Identifiers: D915-D928, D1021-D1025, D1056, and D1057 of Table 3D. In some embodiments, the prime editing composition further comprises a ngRNA that comprises a spacer comprising a sequence selected from the group consisting of SEQ ID NOs. 20548-20561, 20654-20658, 20689, and 20690 of Table 3D.

In some embodiments, the PEgRNA comprises a spacer comprising a sequence selected from the group consisting of SEQ Identifiers: A400-A401 of Table 3A. In some embodiments, the PEgRNA comprises a spacer comprising a sequence as set forth in SEQ ID NOs. 748-749 of Table 3A In some embodiments, the PEgRNA comprises a PBS comprising a sequence selected from the group consisting of SEQ Identifiers: B1656-B1664 of Table 3B. In some embodiments, the PEgRNA comprises a PBS comprising a sequence selected from the group consisting of SEQ ID NOs. 2501-2507 of Table 3B. In some embodiments, the PEgRNA comprises a PBS comprising a sequence selected from the group consisting of sequences AGCAAGAA and AGCAAGAAT as disclosed in Table 3B. In some embodiments, the PEgRNA comprises an editing template comprising a sequence selected from the group consisting of SEQ Identifiers: C7041-C7067 of Table 3C. In some embodiments, the PEgRNA comprises an editing template comprising a sequence selected from the group consisting of SEQ ID NOs: 11067-11093 of Table 3C. In some embodiments, the prime editing composition further comprises a ngRNA that comprises a spacer comprising a sequence selected from the group consisting of SEQ Identifiers: D959, D960, D968-D981, D1002, D1033, D1034, D1035, D1040, D1041, D1058, and D1059 of Table 3D. In some embodiments, the prime editing composition further comprises a ngRNA that comprises a spacer comprising a sequence selected from the group consisting of SEQ ID NOs. 20592, 20593, 20601-20614, 20635, 20666, 20667, 20668, 20673, 20674, 20691, and 20692 of Table 3D.

In some embodiments, the PEgRNA comprises a spacer comprising a sequence selected from the group consisting of SEQ Identifiers: A452-A453 of Table 3A. In some embodiments, the PEgRNA comprises a spacer comprising a sequence as set forth in SEQ ID NOs. 800-801 of Table 3A In some embodiments, the PEgRNA comprises a PBS comprising a sequence selected from the group consisting of SEQ Identifiers: B1881-B1889 of Table 3B. In some embodiments, the PEgRNA comprises a PBS comprising a sequence selected from the group consisting of SEQ ID NOs. 2676-2682 of Table 3B. In some embodiments, the PEgRNA comprises a PBS comprising a sequence selected from the group consisting of sequences GCAAGGTG and GCAAGGTGA as disclosed in Table 3B. In some embodiments, the PEgRNA comprises an editing template comprising a sequence selected from the group consisting of SEQ Identifiers: C7068-C7078 of Table 3C. In some embodiments, the PEgRNA comprises an editing template comprising a sequence selected from the group consisting of SEQ ID NOs: 11094-11104 of Table 3C. In some embodiments, the prime editing composition further comprises a ngRNA that comprises a spacer comprising a sequence selected from the group consisting of SEQ Identifiers: D959, D960, D970-D981, D1002, D1033-D1037, D1040, D1041, and D1055 of Table 3D. In some embodiments, the prime editing composition further comprises a ngRNA that comprises a spacer comprising a sequence selected from the group consisting of SEQ ID NOs. 20592, 20593, 20603-20614, 20635, 20666-20670, 20673, 20674, and 20688 of Table 3D.

In some embodiments, the PEgRNA comprises a spacer comprising a sequence selected from the group consisting of SEQ Identifiers: A454-A455 of Table 3A. In some embodiments, the PEgRNA comprises a spacer comprising a sequence as set forth in SEQ ID NOs. 802-804 of Table 3A In some embodiments, the PEgRNA comprises a PBS comprising a sequence selected from the group consisting of SEQ Identifiers: B1890-B1898 of Table 3B. In some embodiments, the PEgRNA comprises a PBS comprising a sequence selected from the group consisting of SEQ ID NOs. 2682-2689 of Table 3B. In some embodiments, the PEgRNA comprises a PBS comprising a sequence selected from the group consisting of sequences TTCTCCAA and TTCTCCAAG as disclosed in Table 3B. In some embodiments, the PEgRNA comprises an editing template comprising a sequence selected from the group consisting of SEQ Identifiers: C7079-C7101 of Table 3C. In some embodiments, the PEgRNA comprises an editing template comprising a sequence selected from the group consisting of SEQ ID NOs: 11105-11127 of Table 3C. In some embodiments, the prime editing composition further comprises a ngRNA that comprises a spacer comprising a sequence selected from the group consisting of SEQ Identifiers: D929-D944, D946-D952, D1016, D1019, and D1026 of Table 3D. In some embodiments, the prime editing composition further comprises a ngRNA that comprises a spacer comprising a sequence selected from the group consisting of SEQ ID NOs. 20562-20577, 20579-20585, 20649, 20652, and 20659 of Table 3D.

In some embodiments, the PEgRNA comprises a spacer comprising a sequence selected from the group consisting of SEQ Identifiers: A457-A458 of Table 3A. In some embodiments, the PEgRNA comprises a spacer comprising a sequence as set forth in SEQ ID NOs. 805-806 of Table 3A In some embodiments, the PEgRNA comprises a PBS comprising a sequence selected from the group consisting of SEQ Identifiers: B1908-B1916 of Table 3B. In some embodiments, the PEgRNA comprises a PBS comprising a sequence selected from the group consisting of SEQ ID NOs. 2697-2703 of Table 3B. In some embodiments, the PEgRNA comprises a PBS comprising a sequence selected from the group consisting of sequences CAAGGTGA and CAAGGTGAA as disclosed in Table 3B. In some embodiments, the PEgRNA comprises an editing template comprising a sequence selected from the group consisting of SEQ Identifiers: C7102-C7121 of Table 3C. In some embodiments, the PEgRNA comprises an editing template comprising a sequence selected from the group consisting of SEQ ID NOs: 11128-11147 of Table 3C. In some embodiments, the prime editing composition further comprises a ngRNA that comprises a spacer comprising a sequence selected from the group consisting of SEQ Identifiers: D990-D1001 of Table 3D. In some embodiments, the prime editing composition further comprises a ngRNA that comprises a spacer comprising a sequence selected from the group consisting of SEQ ID NOs. 20623-20634 of Table 3D.

In some embodiments, the PEgRNA comprises a spacer comprising the sequence of SEQ Identifier: A409 of Table 3A. In some embodiments, the PEgRNA comprises a spacer comprising a sequence as set forth in SEQ ID NO. 757 of Table 3A In some embodiments, the PEgRNA comprises a PBS comprising a sequence selected from the group consisting of SEQ Identifiers: B1701-B1709 of Table 3B. In some embodiments, the PEgRNA comprises a PBS comprising a sequence selected from the group consisting of SEQ ID NOs. 2536-2542 of Table 3B. In some embodiments, the PEgRNA comprises a PBS comprising a sequence selected from the group consisting of sequences AAGAATTT and AAGAATTTC as disclosed in Table 3B. In some embodiments, the PEgRNA comprises an editing template comprising a sequence selected from the group consisting of SEQ Identifiers: C7122-C7145 of Table 3C. In some embodiments, the PEgRNA comprises an editing template comprising a sequence selected from the group consisting of SEQ ID NOs: 11148-11171 of Table 3C. In some embodiments, the prime editing composition further comprises a ngRNA that comprises a spacer comprising a sequence selected from the group consisting of SEQ Identifiers: D968-D981, D1002, D1033, D1034, and D1035 of Table 3D. In some embodiments, the prime editing composition further comprises a ngRNA that comprises a spacer comprising a sequence selected from the group consisting of SEQ ID NOs. 20601-20614, 20635, 20666, 20667, and 20668 of Table 3D.

In some embodiments, the PEgRNA comprises a spacer comprising a sequence selected from the group consisting of SEQ Identifiers: A475-A476 of Table 3A. In some embodiments, the PEgRNA comprises a spacer comprising a sequence as set forth in SEQ ID NOs. 823-824 of Table 3A In some embodiments, the PEgRNA comprises a PBS comprising a sequence selected from the group consisting of SEQ Identifiers: B1710-B1718 of Table 3B. In some embodiments, the PEgRNA comprises a PBS comprising a sequence selected from the group consisting of SEQ ID NOs. 2543-2549 of Table 3B. In some embodiments, the PEgRNA comprises a PBS comprising a sequence selected from the group consisting of sequences GGAGGTCA and GGAGGTCAA as disclosed in Table 3B. In some embodiments, the PEgRNA comprises an editing template comprising a sequence selected from the group consisting of SEQ Identifiers: C7146-C7207 of Table 3C. In some embodiments, the PEgRNA comprises an editing template comprising a sequence selected from the group consisting of SEQ ID NOs: 11172-11233 of Table 3C. In some embodiments, the prime editing composition further comprises a ngRNA that comprises a spacer comprising a sequence selected from the group consisting of SEQ Identifiers: D988-D992, D997-D1001, and D1060-D1067 of Table 3D. In some embodiments, the prime editing composition further comprises a ngRNA that comprises a spacer comprising a sequence selected from the group consisting of SEQ ID NOs. 20621-20625, 20630-20634, and 20693-20700 of Table 3D.

In some embodiments, the PEgRNA comprises a spacer comprising the sequence of SEQ Identifier: A477 of Table 3A. In some embodiments, the PEgRNA comprises a spacer comprising a sequence as set forth in SEQ ID NO. 825 of Table 3A In some embodiments, the PEgRNA comprises a PBS comprising a sequence selected from the group consisting of SEQ Identifiers: B1719-B1727 of Table 3B. In some embodiments, the PEgRNA comprises a PBS comprising a sequence selected from the group consisting of SEQ ID NOs. 2550-2556 of Table 3B. In some embodiments, the PEgRNA comprises a PBS comprising a sequence selected from the group consisting of sequences TGGAGGTC and TGGAGGTCA as disclosed in Table 3B. In some embodiments, the PEgRNA comprises an editing template comprising a sequence selected from the group consisting of SEQ Identifiers: C7208-C7238 of Table 3C. In some embodiments, the PEgRNA comprises an editing template comprising a sequence selected from the group consisting of SEQ ID NOs: 11234-11264 of Table 3C. In some embodiments, the prime editing composition further comprises a ngRNA that comprises a spacer comprising a sequence selected from the group consisting of SEQ Identifiers: D968-D981, D1002, D1058, D1068, D1069, and D1070 of Table 3D. In some embodiments, the prime editing composition further comprises a ngRNA that comprises a spacer comprising a sequence selected from the group consisting of SEQ ID NOs. 20601-20614, 20635, 21591, 21601, 21602, and 21603 of Table 3D.

In some embodiments, the PEgRNA comprises a spacer comprising a sequence selected from the group consisting of SEQ Identifiers: A478-A480 of Table 3A. In some embodiments, the PEgRNA comprises a spacer comprising a sequence as set forth in SEQ ID NOs. 826-827 of Table 3A In some embodiments, the PEgRNA comprises a PBS comprising a sequence selected from the group consisting of SEQ Identifiers: B1935-B1943 of Table 3B. In some embodiments, the PEgRNA comprises a PBS comprising a sequence selected from the group consisting of SEQ ID NOs. 2718-2724 of Table 3B. In some embodiments, the PEgRNA comprises a PBS comprising a sequence selected from the group consisting of sequences GATTCCAC and GATTCCACC as disclosed in Table 3B. In some embodiments, the PEgRNA comprises an editing template comprising a sequence selected from the group consisting of SEQ Identifiers: C7239-C7269 of Table 3C. In some embodiments, the PEgRNA comprises an editing template comprising a sequence selected from the group consisting of SEQ ID NOs: 11265-11295 of Table 3C. In some embodiments, the prime editing composition further comprises a ngRNA that comprises a spacer comprising a sequence selected from the group consisting of SEQ Identifiers: D925, and D1028-D1032 of Table 3D. In some embodiments, the prime editing composition further comprises a ngRNA that comprises a spacer comprising a sequence selected from the group consisting of SEQ ID NOs. 20558, and 20661-20665 of Table 3D.

In some embodiments, the PEgRNA comprises a spacer comprising a sequence selected from the group consisting of SEQ Identifiers: A416-A417 of Table 3A. In some embodiments, the PEgRNA comprises a spacer comprising a sequence as set forth in SEQ ID NOs. 764-765 of Table 3A In some embodiments, the PEgRNA comprises a PBS comprising a sequence selected from the group consisting of SEQ Identifiers: B1746-B1754 of Table 3B. In some embodiments, the PEgRNA comprises a PBS comprising a sequence selected from the group consisting of SEQ ID NOs. 2571-2577 of Table 3B. In some embodiments, the PEgRNA comprises a PBS comprising a sequence selected from the group consisting of sequences TGTCTTTC and TGTCTTTCT as disclosed in Table 3B. In some embodiments, the PEgRNA comprises an editing template comprising a sequence selected from the group consisting of SEQ Identifiers: C7270-C7281 of Table 3C. In some embodiments, the PEgRNA comprises an editing template comprising a sequence selected from the group consisting of SEQ ID NOs: 11296-11307 of Table 3C. In some embodiments, the prime editing composition further comprises a ngRNA that comprises a spacer comprising a sequence selected from the group consisting of SEQ Identifiers: D915-D928 of Table 3D. In some embodiments, the prime editing composition further comprises a ngRNA that comprises a spacer comprising a sequence selected from the group consisting of SEQ ID NOs. 20548-20561 of Table 3D.

In some embodiments, the PEgRNA comprises a spacer comprising the sequence of SEQ Identifier: A418 of Table 3A. In some embodiments, the PEgRNA comprises a spacer comprising a sequence as set forth in SEQ ID NO. 766 of Table 3A In some embodiments, the PEgRNA comprises a PBS comprising a sequence selected from the group consisting of SEQ Identifiers: B1755-B1763 of Table 3B. In some embodiments, the PEgRNA comprises a PBS comprising a sequence selected from the group consisting of SEQ ID NOs. 2578-2584 of Table 3B. In some embodiments, the PEgRNA comprises a PBS comprising a sequence selected from the group consisting of sequences TTGTCTTT and TTGTCTTTC as disclosed in Table 3B. In some embodiments, the PEgRNA comprises an editing template comprising a sequence selected from the group consisting of SEQ Identifiers: C7282-C7288 of Table 3C. In some embodiments, the PEgRNA comprises an editing template comprising a sequence selected from the group consisting of SEQ ID NOs: 11308-11314 of Table 3C. In some embodiments, the prime editing composition further comprises a ngRNA that comprises a spacer comprising a sequence selected from the group consisting of SEQ Identifiers: D929-D944 of Table 3D. In some embodiments, the prime editing composition further comprises a ngRNA that comprises a spacer comprising a sequence selected from the group consisting of SEQ ID NOs. 20562-20577 of Table 3D.

In some embodiments, the PEgRNA comprises a spacer comprising a sequence selected from the group consisting of SEQ Identifiers: A419-A420 of Table 3A. In some embodiments, the PEgRNA comprises a spacer comprising a sequence as set forth in SEQ ID NOs. 767-768 of Table 3A In some embodiments, the PEgRNA comprises a PBS comprising a sequence selected from the group consisting of Identifiers: B1566-B1574 of Table 3B. In some embodiments, the PEgRNA comprises a PBS comprising a sequence selected from the group consisting of SEQ ID NOs. 2431-2437 of Table 3B. In some embodiments, the PEgRNA comprises a PBS comprising a sequence selected from the group consisting of sequences GAACTATA and GAACTATAT as disclosed in Table 3B. In some embodiments, the PEgRNA comprises an editing template comprising a sequence selected from the group consisting of SEQ Identifiers: C7289-C7316 of Table 3C. In some embodiments, the PEgRNA comprises an editing template comprising a sequence selected from the group consisting of SEQ ID NOs: 11315-11342 of Table 3C. In some embodiments, the prime editing composition further comprises a ngRNA that comprises a spacer comprising a sequence selected from the group consisting of SEQ Identifiers: D915-D928 of Table 3D. In some embodiments, the prime editing composition further comprises a ngRNA that comprises a spacer comprising a sequence selected from the group consisting of SEQ ID NOs. 20548-20561 of Table 3D.

In some embodiments, the PEgRNA comprises a spacer comprising a sequence selected from the group consisting of SEQ Identifiers: A421-A422 of Table 3A. In some embodiments, the PEgRNA comprises a spacer comprising a sequence as set forth in SEQ ID NOs. 769-770 of Table 3A In some embodiments, the PEgRNA comprises a PBS comprising a sequence selected from the group consisting of SEQ Identifiers: B1575-B1583 of Table 3B. In some embodiments, the PEgRNA comprises a PBS comprising a sequence selected from the group consisting of SEQ ID NOs. 2438-2444 of Table 3B. In some embodiments, the PEgRNA comprises a PBS comprising a sequence selected from the group consisting of sequences AGAACTAT and AGAACTATA as disclosed in Table 3B. In some embodiments, the PEgRNA comprises an editing template comprising a sequence selected from the group consisting of SEQ Identifiers: C7317-C7331 of Table 3C. In some embodiments, the PEgRNA comprises an editing template comprising a sequence selected from the group consisting of SEQ ID NOs: 11343-11357 of Table 3C. In some embodiments, the prime editing composition further comprises a ngRNA that comprises a spacer comprising a sequence selected from the group consisting of SEQ Identifiers: D929-D944, D946-D952, and D1016 of Table 3D. In some embodiments, the prime editing composition further comprises a ngRNA that comprises a spacer comprising a sequence selected from the group consisting of SEQ ID NOs. 20562-20577, 20579-20585, and 20649 of Table 3D.

In some embodiments, the PEgRNA comprises a spacer comprising a sequence selected from the group consisting of SEQ Identifiers: A481-A482 of Table 3A. In some embodiments, the PEgRNA comprises a spacer comprising a sequence as set forth in SEQ ID NOs. 829-830 of Table 3A In some embodiments, the PEgRNA comprises a PBS comprising a sequence selected from the group consisting of SEQ Identifiers: B1764-B1772 of Table 3B. In some embodiments, the PEgRNA comprises a PBS comprising a sequence selected from the group consisting of SEQ ID NOs. 2585-2591 of Table 3B. In some embodiments, the PEgRNA comprises a PBS comprising a sequence selected from the group consisting of sequences AGTGTGAT and AGTGTGATT as disclosed in Table 3B. In some embodiments, the PEgRNA comprises an editing template comprising a sequence selected from the group consisting of SEQ Identifiers: C7332-C7362 of Table 3C. In some embodiments, the PEgRNA comprises an editing template comprising a sequence selected from the group consisting of SEQ ID NOs: 11358-11388 of Table 3C. In some embodiments, the prime editing composition further comprises a ngRNA that comprises a spacer comprising a sequence selected from the group consisting of SEQ Identifiers: D930-D944, D1016-D1019, and D1071 of Table 3D. In some embodiments, the prime editing composition further comprises a ngRNA that comprises a spacer comprising a sequence selected from the group consisting of SEQ ID NOs. 20563-20577, 20649-20652, and 20704 of Table 3D.

In some embodiments, the PEgRNA comprises a spacer comprising a sequence selected from the group consisting of SEQ Identifiers: A425-A426 of Table 3A. In some embodiments, the PEgRNA comprises a spacer comprising a sequence as set forth in SEQ ID NOs. 773-774 of Table 3A. In some embodiments, the PEgRNA comprises a PBS comprising a sequence selected from the group consisting of SEQ Identifiers: B1773-B1781 of Table 3B. In some embodiments, the PEgRNA comprises a PBS comprising a sequence selected from the group consisting of SEQ ID NOs. 2952-2958 of Table 3B. In some embodiments, the PEgRNA comprises a PBS comprising a sequence selected from the group consisting of sequences CCAAGAAC and CCAAGAACT as disclosed in Table 3B. In some embodiments, the PEgRNA comprises an editing template comprising a sequence selected from the group consisting of SEQ Identifiers: C7363-C7380 of Table 3C. In some embodiments, the PEgRNA comprises an editing template comprising a sequence selected from the group consisting of SEQ ID NOs: 11389-11406 of Table 3C. In some embodiments, the prime editing composition further comprises a ngRNA that comprises a spacer comprising a sequence selected from the group consisting of SEQ Identifiers: D929-D944, and D1016-D1018 of Table 3D. In some embodiments, the prime editing composition further comprises a ngRNA that comprises a spacer comprising a sequence selected from the group consisting of SEQ ID NOs. 20562-20577, and 20649-20651 of Table 3D.

In some embodiments, the PEgRNA comprises a spacer comprising the sequence of SEQ Identifier: A427 of Table 3A. In some embodiments, the PEgRNA comprises a spacer comprising a sequence as set forth in SEQ ID NO.775 of Table 3A. In some embodiments, the PEgRNA comprises a PBS comprising a sequence selected from the group consisting of SEQ Identifiers: B1782-B1790 of Table 3B. In some embodiments, the PEgRNA comprises a PBS comprising a sequence selected from the group consisting of SEQ ID NOs. 2599-2605 of Table 3B. In some embodiments, the PEgRNA comprises a PBS comprising a sequence selected from the group consisting of sequences TCCAAGAA and TCCAAGAAC as disclosed in Table 3B. In some embodiments, the PEgRNA comprises an editing template comprising a sequence selected from the group consisting of SEQ Identifiers: C7381-C7399 of Table 3C. In some embodiments, the PEgRNA comprises an editing template comprising a sequence selected from the group consisting of SEQ ID NOs: 11407-11425 of Table 3C. In some embodiments, the prime editing composition further comprises a ngRNA that comprises a spacer comprising a sequence selected from the group consisting of SEQ Identifiers: D929-D944, D1016, and D1019 of Table 3D. In some embodiments, the prime editing composition further comprises a ngRNA that comprises a spacer comprising a sequence selected from the group consisting of SEQ ID NOs. 0.20562-20577, 20649, and 20652 of Table 3D.

In some embodiments, the PEgRNA comprises a spacer comprising a sequence selected from the group consisting of SEQ Identifier: A428-A429 of Table 3A. In some embodiments, the PEgRNA comprises a spacer comprising a sequence as set forth in SEQ ID NOs. 776-777 of Table 3A. In some embodiments, the PEgRNA comprises a PBS comprising a sequence selected from the group consisting of SEQ Identifiers: B1791-B1799 of Table 3B. In some embodiments, the PEgRNA comprises a PBS comprising a sequence selected from the group consisting of SEQ ID NOs. 2606-2612 of Table 3B. In some embodiments, the PEgRNA comprises a PBS comprising a sequence selected from the group consisting of sequences ACTATATT and ACTATATTG as disclosed in Table 3B. In some embodiments, the PEgRNA comprises an editing template comprising a sequence selected from the group consisting of SEQ Identifiers: C7400-C7411 of Table 3C. In some embodiments, the PEgRNA comprises an editing template comprising a sequence selected from the group consisting of SEQ ID NOs: 11426-11437 of Table 3C. In some embodiments, the prime editing composition further comprises a ngRNA that comprises a spacer comprising a sequence selected from the group consisting of SEQ Identifiers: D929-D944, and D1016-D1018 of Table 3D. In some embodiments, the prime editing composition further comprises a ngRNA that comprises a spacer comprising a sequence selected from the group consisting of SEQ ID NOs. 20562-20577, and 20649-20651 of Table 3D.

In some embodiments, the PEgRNA comprises a spacer comprising a sequence selected from the group consisting of SEQ Identifiers: A451, A483, A484, and A485 of Table 3A. In some embodiments, the PEgRNA comprises a spacer comprising a sequence as set forth in SEQ ID NOs. 799 and 831-833 of Table 3A. In some embodiments, the PEgRNA comprises a PBS comprising a sequence selected from the group consisting of SEQ Identifiers: B1872-B1880 of Table 3B. In some embodiments, the PEgRNA comprises a PBS comprising a sequence selected from the group consisting of SEQ ID NOs. 2669-2675 of Table 3B. In some embodiments, the PEgRNA comprises a PBS comprising a sequence selected from the group consisting of sequences TGTGATTC and TGTGATTCC as disclosed in Table 3B. In some embodiments, the PEgRNA comprises an editing template comprising a sequence selected from the group consisting of SEQ Identifiers: C7412-C7442 of Table 3C. In some embodiments, the PEgRNA comprises an editing template comprising a sequence selected from the group consisting of SEQ ID NOs: 11438-11468 of Table 3C. In some embodiments, the prime editing composition further comprises a ngRNA that comprises a spacer comprising a sequence selected from the group consisting of SEQ Identifiers: D922, D930-D944, D1016-1019, and D1022-D1025 of Table 3D. In some embodiments, the prime editing composition further comprises a ngRNA that comprises a spacer comprising a sequence selected from the group consisting of SEQ ID NOs. 20555, 20563-20577, 20649-20652, and 20655-20658 of Table 3D.

In some embodiments, the PEgRNA comprises a spacer comprising a sequence selected from the group consisting of SEQ Identifiers: A433-A435 of Table 3A. In some embodiments, the PEgRNA comprises a spacer comprising a sequence as set forth in SEQ ID NOs. 781-782 of Table 3A. In some embodiments, the PEgRNA comprises a PBS comprising a sequence selected from the group consisting of SEQ Identifiers: B1809-B1817 of Table 3B. In some embodiments, the PEgRNA comprises a PBS comprising a sequence selected from the group consisting of SEQ ID NOs. 2620-2626 of Table 3B. In some embodiments, the PEgRNA comprises a PBS comprising a sequence selected from the group consisting of sequences CTATATTG and CTATATTGT as disclosed in Table 3B. In some embodiments, the PEgRNA comprises an editing template comprising a sequence selected from the group consisting of SEQ Identifiers: C7443-C7453 of Table 3C. In some embodiments, the PEgRNA comprises an editing template comprising a sequence selected from the group consisting of SEQ ID NOs: 11469-11479 of Table 3C. In some embodiments, the prime editing composition further comprises a ngRNA that comprises a spacer comprising a sequence selected from the group consisting of SEQ Identifiers: D922, and D1021-D1025 of Table 3D. In some embodiments, the prime editing composition further comprises a ngRNA that comprises a spacer comprising a sequence selected from the group consisting of SEQ ID NOs. 20555, and 20654-20658 of Table 3D.

In some embodiments, the PEgRNA comprises a spacer comprising a sequence selected from the group consisting of SEQ Identifiers: A486-A487 of Table 3A. In some embodiments, the PEgRNA comprises a spacer comprising a sequence as set forth in SEQ ID NOs. 834-835 of Table 3A. In some embodiments, the PEgRNA comprises a PBS comprising a sequence selected from the group consisting of SEQ Identifiers: B1818-B1826 of Table 3B. In some embodiments, the PEgRNA comprises a PBS comprising a sequence selected from the group consisting of SEQ ID NOs. 2627-2633 of Table 3B. In some embodiments, the PEgRNA comprises a PBS comprising a sequence selected from the group consisting of sequences CAGTGTGA and CAGTGTGAT as disclosed in Table 3B. In some embodiments, the PEgRNA comprises an editing template comprising a sequence selected from the group consisting of SEQ Identifiers: C7454-C7484 of Table 3C. In some embodiments, the PEgRNA comprises an editing template comprising a sequence selected from the group consisting of SEQ ID NOs: 11480-11510 of Table 3C. In some embodiments, the prime editing composition further comprises a ngRNA that comprises a spacer comprising a sequence selected from the group consisting of SEQ Identifiers: D930-D944, D946-D952, D1016, D1019, D1026, D1071, and D1072 of Table 3D. In some embodiments, the prime editing composition further comprises a ngRNA that comprises a spacer comprising a sequence selected from the group consisting of SEQ ID NOs. 20563-20577, 20579-20585, 20649, 20652, 20659, 20704, and 20705 of Table 3D.

In some embodiments, the PEgRNA comprises a spacer comprising a sequence selected from the group consisting of SEQ Identifiers: A438-A441 of Table 3A. In some embodiments, the PEgRNA comprises a spacer comprising a sequence as set forth in SEQ ID NOs. 786-789 of Table 3A. In some embodiments, the PEgRNA comprises a PBS comprising a sequence selected from the group consisting of SEQ Identifiers: B1827-B1835 of Table 3B. In some embodiments, the PEgRNA comprises a PBS comprising a sequence selected from the group consisting of SEQ ID NOs. 2634-2640 of Table 3B. In some embodiments, the PEgRNA comprises a PBS comprising a sequence selected from the group consisting of sequences TCTCCAAG and TCTCCAAGA as disclosed in Table 3B. In some embodiments, the PEgRNA comprises an editing template comprising a sequence selected from the group consisting of SEQ Identifiers: C7485-C7505 of Table 3C. In some embodiments, the PEgRNA comprises an editing template comprising a sequence selected from the group consisting of SEQ ID NOs: 11511-11531 of Table 3C. In some embodiments, the prime editing composition further comprises a ngRNA that comprises a spacer comprising a sequence selected from the group consisting of SEQ Identifiers: D925, D929-D944, D1016-D1019, and D1028-D1032 of Table 3D. In some embodiments, the prime editing composition further comprises a ngRNA that comprises a spacer comprising a sequence selected from the group consisting of SEQ ID NOs. 20558, 20562-20577, 20649-20652, and 20661-20665 of Table 3D.

In some embodiments, the PEgRNA comprises a spacer comprising the sequence of SEQ Identifier: A442 of Table 3A. In some embodiments, the PEgRNA comprises a spacer comprising a sequence as set forth in SEQ ID NO. 790 of Table 3A. In some embodiments, the PEgRNA comprises a PBS comprising a sequence selected from the group consisting of SEQ Identifiers: B1836-B1844 of Table 3B. In some embodiments, the PEgRNA comprises a PBS comprising a sequence selected from the group consisting of SEQ ID NOs. 2641-2647 of Table 3B. In some embodiments, the PEgRNA comprises a PBS comprising a sequence selected from the group consisting of sequences TGAATAA and TGAATAACT as disclosed in Table 3B. In some embodiments, the PEgRNA comprises an editing template comprising a sequence selected from the group consisting of SEQ Identifiers: C7506-C7511 of Table 3C. In some embodiments, the PEgRNA comprises an editing template comprising a sequence selected from the group consisting of SEQ ID NOs: 11532-11537 of Table 3C. In some embodiments, the prime editing composition further comprises a ngRNA that comprises a spacer comprising a sequence selected from the group consisting of SEQ Identifiers: D972-D981, D1002, D1033-D1037, D1055, and D1073 of Table 3D. In some embodiments, the prime editing composition further comprises a ngRNA that comprises a spacer comprising a sequence selected from the group consisting of SEQ ID NOs. 20605-20614, 20635, 20666-20670, 20688, and 20706 of Table 3D.

In some embodiments, the PEgRNA comprises a spacer comprising a sequence selected from the group consisting of SEQ Identifiers: A443-A445 of Table 3A. In some embodiments, the PEgRNA comprises a spacer comprising a sequence as set forth in SEQ ID NOs. 791-793 of Table 3A. In some embodiments, the PEgRNA comprises a PBS comprising a sequence selected from the group consisting of SEQ Identifiers: B1845-B1853 of Table 3B. In some embodiments, the PEgRNA comprises a PBS comprising a sequence selected from the group consisting of SEQ ID NOs. 2648-2654 of Table 3B. In some embodiments, the PEgRNA comprises a PBS comprising a sequence selected from the group consisting of sequences CTCCAAGA and CTCCAAGAA as disclosed in Table 3B. In some embodiments, the PEgRNA comprises an editing template comprising a sequence selected from the group consisting of SEQ Identifiers: C7512-C7531 of Table 3C In some embodiments, the PEgRNA comprises an editing template comprising a sequence selected from the group consisting of SEQ ID NOs: 11538-11557 of Table 3C. In some embodiments, the prime editing composition further comprises a ngRNA that comprises a spacer comprising a sequence selected from the group consisting of SEQ Identifiers: D922, and D1021-D1025 of Table 3D. In some embodiments, the prime editing composition further comprises a ngRNA that comprises a spacer comprising a sequence selected from the group consisting of SEQ ID NOs. 20555, and 20654-20658 of Table 3D.

In some embodiments, the PEgRNA comprises a spacer comprising the sequence of SEQ Identifier: A446 of Table 3A. In some embodiments, the PEgRNA comprises a spacer comprising a sequence as set forth in SEQ ID NO. 794 of Table 3A. In some embodiments, the PEgRNA comprises a PBS comprising a sequence selected from the group consisting of SEQ Identifiers: B1854-B1862 of Table 3B. In some embodiments, the PEgRNA comprises a PBS comprising a sequence selected from the group consisting of SEQ ID NOs. 2655-2661 of Table 3B. In some embodiments, the PEgRNA comprises a PBS comprising a sequence selected from the group consisting of sequences ATTTCTTT and ATTTCTTTA as disclosed in Table 3B. In some embodiments, the PEgRNA comprises an editing template comprising a sequence selected from the group consisting of SEQ Identifiers: C7532-C7552 of Table 3C. In some embodiments, the PEgRNA comprises an editing template comprising a sequence selected from the group consisting of SEQ ID NOs: 11558-11578 of Table 3C. In some embodiments, the prime editing composition further comprises a ngRNA that comprises a spacer comprising a sequence selected from the group consisting of SEQ Identifiers: D968-D981, D1002, D1033, D1034, and D1035 of Table 3D. In some embodiments, the prime editing composition further comprises a ngRNA that comprises a spacer comprising a sequence selected from the group consisting of SEQ ID NOs. 20601-20614, 20635, 20666, 20667, and 20668 of Table 3D.

In some embodiments, the PEgRNA comprises a spacer comprising a sequence selected from the group consisting of SEQ Identifiers: A391-A392 of Table 3A. In some embodiments, the PEgRNA comprises a spacer comprising a sequence as set forth in SEQ ID NOs. 739-740 of Table 3A. In some embodiments, the PEgRNA comprises a PBS comprising a sequence selected from the group consisting of SEQ Identifiers: B1611-B1619 of Table 3B. In some embodiments, the PEgRNA comprises a PBS comprising a sequence selected from the group consisting of SEQ ID NOs. 2466-2472 of Table 3B. In some embodiments, the PEgRNA comprises a PBS comprising a sequence selected from the group consisting of sequences AACTATAT and AACTATATT as disclosed in Table 3B. In some embodiments, the PEgRNA comprises an editing template comprising a sequence selected from the group consisting of SEQ Identifiers: C7553-C7565 of Table 3C. In some embodiments, the PEgRNA comprises an editing template comprising a sequence selected from the group consisting of SEQ ID NOs: 11579-11591 of Table 3C. In some embodiments, the prime editing composition further comprises a ngRNA that comprises a spacer comprising a sequence selected from the group consisting of SEQ Identifiers: D929-D944, D946-D952, and D1016 of Table 3D. In some embodiments, the prime editing composition further comprises a ngRNA that comprises a spacer comprising a sequence selected from the group consisting of SEQ ID NOs. 20562-20577, 20579-20585, and 20649 of Table 3D.

In some embodiments, the PEgRNA comprises a spacer comprising a sequence selected from the group consisting of SEQ Identifier: A465, A466, A467, and A488 of Table 3A. In some embodiments, the PEgRNA comprises a spacer comprising a sequence as set forth in SEQ ID NOs. 813-816 and 836 of Table 3A. In some embodiments, the PEgRNA comprises a PBS comprising a sequence selected from the group consisting of SEQ Identifiers: B1800-B1808 of Table 3B. In some embodiments, the PEgRNA comprises a PBS comprising a sequence selected from the group consisting of SEQ ID NOs. 2613-2619 of Table 3B. In some embodiments, the PEgRNA comprises a PBS comprising a sequence selected from the group consisting of sequences GTGTGATT and GTGTGATTC as disclosed in Table 3B. In some embodiments, the PEgRNA comprises an editing template comprising a sequence selected from the group consisting of SEQ Identifiers: C7566-C7596 of Table 3C. In some embodiments, the PEgRNA comprises an editing template comprising a sequence selected from the group consisting of SEQ ID NOs: 11592-11622 of Table 3C. In some embodiments, the prime editing composition further comprises a ngRNA that comprises a spacer comprising a sequence selected from the group consisting of SEQ Identifiers: D922, D930-D944, D1016-D1019, and D1021-D1025 of Table 3D. In some embodiments, the prime editing composition further comprises a ngRNA that comprises a spacer comprising a sequence selected from the group consisting of SEQ ID NOs. 20555, 20563-20577, 20649-20652, and 20654-20658 of Table 3D.

In some embodiments, the PEgRNA comprises a spacer comprising a sequence selected from the group consisting of SEQ Identifiers: A447-A450 of Table 3A. In some embodiments, the PEgRNA comprises a spacer comprising a sequence as set forth in SEQ ID NOs. 795-798 of Table 3A. In some embodiments, the PEgRNA comprises a PBS comprising a sequence selected from the group consisting of SEQ Identifiers: B1863-B1871 of Table 3B. In some embodiments, the PEgRNA comprises a PBS comprising a sequence selected from the group consisting of SEQ ID NOs. 2662-2668 of Table 3B. In some embodiments, the PEgRNA comprises a PBS comprising a sequence selected from the group consisting of sequences CAAGAACT and CAAGAACTA asdisclosed in Table 3B. In some embodiments, the PEgRNA comprises an editing template comprising a sequence selected from the group consisting of SEQ Identifiers: C7597-C7630 of Table 3C. In some embodiments, the PEgRNA comprises an editing template comprising a sequence selected from the group consisting of SEQ ID NOs: 11623-11656 of Table 3C. In some embodiments, the prime editing composition further comprises a ngRNA that comprises a spacer comprising a sequence selected from the group consisting of SEQ Identifiers: D915-D928, D1021-D1025 of Table 3D. In some embodiments, the prime editing composition further comprises a ngRNA that comprises a spacer comprising a sequence selected from the group consisting of SEQ ID NOs. 20548-20561, 20654-20658 of Table 3D.

In some embodiments, the PEgRNA comprises a spacer comprising a sequence selected from the group consisting of SEQ Identifiers: A489-A492 of Table 3A. In some embodiments, the PEgRNA comprises a spacer comprising a sequence as set forth in SEQ ID NOs. 837-840 of Table 3A. In some embodiments, the PEgRNA comprises a PBS comprising a sequence selected from the group consisting of SEQ Identifiers: B1944-B1952 of Table 3B. In some embodiments, the PEgRNA comprises a PBS comprising a sequence selected from the group consisting of SEQ ID NOs. 2725-2731 of Table 3B. In some embodiments, the PEgRNA comprises a PBS comprising a sequence selected from the group consisting of sequences GTGATTCC and GTGATTCCA as disclosed in Table 3B. In some embodiments, the PEgRNA comprises an editing template comprising a sequence selected from the group consisting of SEQ Identifiers: C7631-C7692 of Table 3C. In some embodiments, the PEgRNA comprises an editing template comprising a sequence selected from the group consisting of SEQ ID NOs: 11657-11718 of Table 3C. In some embodiments, the prime editing composition further comprises a ngRNA that comprises a spacer comprising a sequence selected from the group consisting of SEQ Identifiers: D915-D928, D1021-D1025, D1056, and D1057 of Table 3D. In some embodiments, the prime editing composition further comprises a ngRNA that comprises a spacer comprising a sequence selected from the group consisting of SEQ ID NOs. 20548-20561, 20654-20658, 20689, and 20690 of Table 3D.

In some embodiments, the PEgRNA comprises a spacer comprising a sequence selected from the group consisting of SEQ Identifiers: A456 and A470 of Table 3A. In some embodiments, the PEgRNA comprises a spacer comprising a sequence as set forth in SEQ ID NOs. 804 and 818 of Table 3A. In some embodiments, the PEgRNA comprises a PBS comprising a sequence selected from the group consisting of SEQ Identifiers: B1899-B1907 of Table 3B. In some embodiments, the PEgRNA comprises a PBS comprising a sequence selected from the group consisting of SEQ ID NOs. 2690-2696 of Table 3B. In some embodiments, the PEgRNA comprises a PBS comprising a sequence selected from the group consisting of sequences TGATTCCA and TGATTCCAC as disclosed in Table 3B. In some embodiments, the PEgRNA comprises an editing template comprising a sequence selected from the group consisting of SEQ Identifiers: C7693-C7723 of Table 3C. In some embodiments, the PEgRNA comprises an editing template comprising a sequence selected from the group consisting of SEQ ID NOs: 11719-11749 of Table 3C. In some embodiments, the prime editing composition further comprises a ngRNA that comprises a spacer comprising a sequence selected from the group consisting of SEQ Identifiers: D930-D944, D946-D952, D1016, D1019, and D1026 of Table 3D. In some embodiments, the prime editing composition further comprises a ngRNA that comprises a spacer comprising a sequence selected from the group consisting of SEQ ID NOs. 20563-20577, 20579-20585, 20649, 20652, and 20659 of Table 3D.

In some embodiments, the PEgRNA comprises a spacer comprising a sequence selected from the group consisting of SEQ Identifiers: A493-A496 of Table 3A. In some embodiments, the PEgRNA comprises a spacer comprising a sequence as set forth in SEQ ID NOs. 841-844 of Table 3A. In some embodiments, the PEgRNA comprises a PBS comprising a sequence selected from the group consisting of SEQ Identifiers: B1926-B1934 of Table 3B. In some embodiments, the PEgRNA comprises a PBS comprising a sequence selected from the group consisting of SEQ ID NOs. 2711-2717 of Table 3B. In some embodiments, the PEgRNA comprises a PBS comprising a sequence selected from the group consisting of sequences TCAGTGTG and TCAGTGTGA as disclosed in Table 3B. In some embodiments, the PEgRNA comprises an editing template comprising a sequence selected from the group consisting of SEQ Identifiers: C7724-C7785 of Table 3C. In some embodiments, the PEgRNA comprises an editing template comprising a sequence selected from the group consisting of SEQ ID NOs: 11750-11811 of Table 3C. In some embodiments, the prime editing composition further comprises a ngRNA that comprises a spacer comprising a sequence selected from the group consisting of SEQ Identifiers: D915-D928, D1021-D1025, D1056, and D1057 of Table 3D. In some embodiments, the prime editing composition further comprises a ngRNA that comprises a spacer comprising a sequence selected from the group consisting of SEQ ID NOs. 20548-20561, 20654-20658, 20689, and 20690 of Table 3D.

In some embodiments, the PEgRNA comprises a spacer comprising a sequence selected from the group consisting of SEQ Identifiers: A497-A498 of Table 3A. In some embodiments, the PEgRNA comprises a spacer comprising a sequence as set forth in SEQ ID NOs. 845-846 of Table 3A. In some embodiments, the PEgRNA comprises a PBS comprising a sequence selected from the group consisting of SEQ Identifiers: B1953-B1961 of Table 3B. In some embodiments, the PEgRNA comprises a PBS comprising a sequence selected from the group consisting of SEQ ID NOs. 2732-2738 of Table 3B. In some embodiments, the PEgRNA comprises a PBS comprising a sequence selected from the group consisting of sequences CTCAGTGT and CTCAGTGTG as disclosed in Table 3B. In some embodiments, the PEgRNA comprises an editing template comprising a sequence selected from the group consisting of SEQ Identifiers: C7786-C7816 of Table 3C. In some embodiments, the PEgRNA comprises an editing template comprising a sequence selected from the group consisting of SEQ ID NOs: 11812-11842 of Table 3C. In some embodiments, the prime editing composition further comprises a ngRNA that comprises a spacer comprising a sequence selected from the group consisting of SEQ Identifiers: D930-D944, D1016-D1019, D1071, and D1074 of Table 3D. In some embodiments, the prime editing composition further comprises a ngRNA that comprises a spacer comprising a sequence selected from the group consisting of SEQ ID NOs. 20563-20577, 20649, -20652, 20704, and 20707 of Table 3D.

In some embodiments, the PEgRNA comprises a spacer comprising a sequence selected from the group consisting of SEQ Identifiers: A400-A401 of Table 3A. In some embodiments, the PEgRNA comprises a spacer comprising a sequence as set forth in SEQ ID NOs. 748-749 of Table 3A. In some embodiments, the PEgRNA comprises a PBS comprising a sequence selected from the group consisting of SEQ Identifiers: B1656-B1664 of Table 3B. In some embodiments, the PEgRNA comprises a PBS comprising a sequence selected from the group consisting of SEQ ID NOs. 2501-2507 of Table 3B. In some embodiments, the PEgRNA comprises a PBS comprising a sequence selected from the group consisting of sequences AGCAAGAA and AGCAAGAAT as disclosed in Table 3B. In some embodiments, the PEgRNA comprises an editing template comprising a sequence selected from the group consisting of SEQ Identifiers: C7817-C7844 of Table 3C. In some embodiments, the PEgRNA comprises an editing template comprising a sequence selected from the group consisting of SEQ ID NOs: 11843-11870 of Table 3C. In some embodiments, the prime editing composition further comprises a ngRNA that comprises a spacer comprising a sequence selected from the group consisting of SEQ Identifiers: D959, D960, D968-D981, D1002, D1033, D1034, D1035, D1040, D1041, D1075, and D1076 of Table 3D. In some embodiments, the prime editing composition further comprises a ngRNA that comprises a spacer comprising a sequence selected from the group consisting of SEQ ID NOs. 20592, 20593, 20601-20614, 20635, 20666, 20667, 20668, 20673, 20674, 20708, and 20709 of Table 3D.

In some embodiments, the PEgRNA comprises a spacer comprising a sequence selected from the group consisting of SEQ Identifiers: A452-A453 of Table 3A. In some embodiments, the PEgRNA comprises a spacer comprising a sequence as set forth in SEQ ID NOs. 800-801 of Table 3A. In some embodiments, the PEgRNA comprises a PBS comprising a sequence selected from the group consisting of SEQ Identifiers: B1881-B1889 of Table 3B. In some embodiments, the PEgRNA comprises a PBS comprising a sequence selected from the group consisting of SEQ ID NOs. 2676-2682 of Table 3B. In some embodiments, the PEgRNA comprises a PBS comprising a sequence selected from the group consisting of sequences GCAAGGTG and GCAAGGTGA as disclosed in Table 3B. In some embodiments, the PEgRNA comprises an editing template comprising a sequence selected from the group consisting of SEQ Identifiers: C7845-C7856 of Table 3C. In some embodiments, the PEgRNA comprises an editing template comprising a sequence selected from the group consisting of SEQ ID NOs: 11871-11882 of Table 3C. In some embodiments, the prime editing composition further comprises a ngRNA that comprises a spacer comprising a sequence selected from the group consisting of SEQ Identifiers: D959-D960, D970-D981, D1002, D1033, D1034, D1035, D1036, D1037, D1040, D1041, D1055, and D1073 of Table 3D. In some embodiments, the prime editing composition further comprises a ngRNA that comprises a spacer comprising a sequence selected from the group consisting of SEQ ID NOs. 20592-20593, 20603-20614, 20635, 20666, 20667, 20668, 20673, 20674, 20688, and 20706 of Table 3D.

In some embodiments, the PEgRNA comprises a spacer comprising a sequence selected from the group consisting of SEQ Identifiers: A454-A455 of Table 3A. In some embodiments, the PEgRNA comprises a spacer comprising a sequence as set forth in SEQ ID NOs. 802-803 of Table 3A. In some embodiments, the PEgRNA comprises a PBS comprising a sequence selected from the group consisting of SEQ Identifiers: B1890-B1898 of Table 3B. In some embodiments, the PEgRNA comprises a PBS comprising a sequence selected from the group consisting of SEQ ID NOs. 2683-2689 of Table 3B. In some embodiments, the PEgRNA comprises a PBS comprising a sequence selected from the group consisting of sequences TTCTCCAA and TTCTCCAAG as disclosed in Table 3B. In some embodiments, the PEgRNA comprises an editing template comprising a sequence selected from the group consisting of SEQ Identifiers: C7857-C7878 of Table 3C. In some embodiments, the PEgRNA comprises an editing template comprising a sequence selected from the group consisting of SEQ ID NOs: 11883-11894 of Table 3C. In some embodiments, the prime editing composition further comprises a ngRNA that comprises a spacer comprising a sequence selected from the group consisting of SEQ Identifiers: D929-D944, D946-D952, D1016, D1019, and D1026 of Table 3D. In some embodiments, the prime editing composition further comprises a ngRNA that comprises a spacer comprising a sequence selected from the group consisting of SEQ ID NOs. 20562-20577, 20579-20583, 20649, 20652, and 20659 of Table 3D.

In some embodiments, the PEgRNA comprises a spacer comprising a sequence selected from the group consisting of SEQ Identifiers: A457-A458 of Table 3A. In some embodiments, the PEgRNA comprises a spacer comprising a sequence as set forth in SEQ ID NOs. 805-806 of Table 3A. In some embodiments, the PEgRNA comprises a PBS comprising a sequence selected from the group consisting of SEQ Identifiers: B1908-B1916 of Table 3B. In some embodiments, the PEgRNA comprises a PBS comprising a sequence selected from the group consisting of SEQ ID NOs. 2697-2703 of Table 3B. In some embodiments, the PEgRNA comprises a PBS comprising a sequence selected from the group consisting of sequences CAAGGTGA and CAAGGTGAA as disclosed in Table 3B. In some embodiments, the PEgRNA comprises an editing template comprising a sequence selected from the group consisting of SEQ Identifiers: C7879-C7900 of Table 3C. In some embodiments, the PEgRNA comprises an editing template comprising a sequence selected from the group consisting of SEQ ID NOs: 11905-11926 of Table 3C. In some embodiments, the prime editing composition further comprises a ngRNA that comprises a spacer comprising a sequence selected from the group consisting of SEQ Identifiers: D990, D991, D992, D999, D1000, and D1001. In some embodiments, the prime editing composition further comprises a ngRNA that comprises a spacer comprising a sequence selected from the group consisting of SEQ ID NOs. 20623, 20624, 20625, 20632, 20633, and 20634 of Table 3D.

In some embodiments, the PEgRNA comprises a spacer comprising the sequence of SEQ Identifier: A409 of Table 3A. In some embodiments, the PEgRNA comprises a spacer comprising a sequence as set forth in SEQ ID NO. 757 of Table 3A. In some embodiments, the PEgRNA comprises a PBS comprising a sequence selected from the group consisting of SEQ Identifiers: B1701-B1709 of Table 3B. In some embodiments, the PEgRNA comprises a PBS comprising a sequence selected from the group consisting of SEQ ID NOs. 2536-2542 of Table 3B. In some embodiments, the PEgRNA comprises a PBS comprising a sequence selected from the group consisting of sequences AAGAATTT and AAGAATTTC as disclosed in Table 3B. In some embodiments, the PEgRNA comprises an editing template comprising a sequence selected from the group consisting of SEQ Identifiers: C7901-C7925 of Table 3C. In some embodiments, the PEgRNA comprises an editing template comprising a sequence selected from the group consisting of SEQ ID NOs: 11927-11951 of Table 3C. In some embodiments, the prime editing composition further comprises a ngRNA that comprises a spacer comprising a sequence selected from the group consisting of SEQ Identifiers: D968-D981, D1002, D1033, D1034, and D1035 of Table 3D. In some embodiments, the prime editing composition further comprises a ngRNA that comprises a spacer comprising a sequence selected from the group consisting of SEQ ID NOs. 20601-20614, 20636, 20666, 20667, and 20668 of Table 3D.

In some embodiments, the PEgRNA comprises a spacer comprising a sequence selected from the group consisting of SEQ Identifiers: A499-A500 of Table 3A. In some embodiments, the PEgRNA comprises a spacer comprising a sequence as set forth in SEQ ID NOs. 847-848 of Table 3A. In some embodiments, the PEgRNA comprises a PBS comprising a sequence selected from the group consisting of SEQ Identifiers: B1710-B1718 of Table 3B. In some embodiments, the PEgRNA comprises a PBS comprising a sequence selected from the group consisting of SEQ ID NOs. 2543-2549 of Table 3B. In some embodiments, the PEgRNA comprises a PBS comprising a sequence selected from the group consisting of sequences GGAGGTCA and GGAGGTCAA as disclosed in Table 3B. In some embodiments, the PEgRNA comprises an editing template comprising a sequence selected from the group consisting of SEQ Identifiers: C7926-C7987 of Table 3C. In some embodiments, the PEgRNA comprises an editing template comprising a sequence selected from the group consisting of SEQ ID NOs: 11952-12013 of Table 3C. In some embodiments, the prime editing composition further comprises a ngRNA that comprises a spacer comprising a sequence selected from the group consisting of SEQ Identifiers: D988-D992, D997-D1001, and D1077-D1084 of Table 3D. In some embodiments, the prime editing composition further comprises a ngRNA that comprises a spacer comprising a sequence selected from the group consisting of SEQ ID NOs. 20621-20625, 20630-20634, and 20710-20717 of Table 3D.

In some embodiments, the PEgRNA comprises a spacer comprising a sequence selected from the group consisting of SEQ Identifiers: A416-A417 of Table 3A. In some embodiments, the PEgRNA comprises a spacer comprising a sequence as set forth in SEQ ID NOs. 464-465 of Table 3A. In some embodiments, the PEgRNA comprises a PBS comprising a sequence selected from the group consisting of SEQ Identifiers: B1746-B1754 of Table 3B. In some embodiments, the PEgRNA comprises a PBS comprising a sequence selected from the group consisting of SEQ ID NOs. 2571-2577 of Table 3B. In some embodiments, the PEgRNA comprises a PBS comprising a sequence selected from the group consisting of sequences TGTCTTTC and TGTCTTTCT as disclosed in Table 3B. In some embodiments, the PEgRNA comprises an editing template comprising a sequence selected from the group consisting of SEQ Identifiers: C7988-C7997 of Table 3C. In some embodiments, the PEgRNA comprises an editing template comprising a sequence selected from the group consisting of SEQ ID NOs: 12014-12023 of Table 3C. In some embodiments, the prime editing composition further comprises a ngRNA that comprises a spacer comprising a sequence selected from the group consisting of SEQ Identifiers: D915-D928 of Table 3D. In some embodiments, the prime editing composition further comprises a ngRNA that comprises a spacer comprising a sequence selected from the group consisting of SEQ ID NOs. 20548-20561 of Table 3D.

In some embodiments, the PEgRNA comprises a spacer comprising the sequence of SEQ Identifier: A418 of Table 3A. In some embodiments, the PEgRNA comprises a spacer comprising a sequence as set forth in SEQ ID NO. 766 of Table 3A. In some embodiments, the PEgRNA comprises a PBS comprising a sequence selected from the group consisting of SEQ Identifiers: B1755-B1763 of Table 3B. In some embodiments, the PEgRNA comprises a PBS comprising a sequence selected from the group consisting of SEQ ID NOs. 2578-2584 of Table 3B. In some embodiments, the PEgRNA comprises a PBS comprising a sequence selected from the group consisting of sequences TTGTCTTT and TTGTCTTTC as disclosed in Table 3B. In some embodiments, the PEgRNA comprises an editing template comprising a sequence selected from the group consisting of SEQ Identifiers: C7998-C8003 of Table 3C. In some embodiments, the PEgRNA comprises an editing template comprising a sequence selected from the group consisting of SEQ ID NOs: 12024-12029 of Table 3C. In some embodiments, the prime editing composition further comprises a ngRNA that comprises a spacer comprising a sequence selected from the group consisting of SEQ Identifiers: D929-D944 of Table 3D. In some embodiments, the prime editing composition further comprises a ngRNA that comprises a spacer comprising a sequence selected from the group consisting of SEQ ID NOs. 20562-20577 of Table 3D.

In some embodiments, the PEgRNA comprises a spacer comprising a sequence selected from the group consisting of SEQ Identifiers: A419-A420 of Table 3A. In some embodiments, the PEgRNA comprises a spacer comprising a sequence as set forth in SEQ ID NOs. 767-768 of Table 3A. In some embodiments, the PEgRNA comprises a PBS comprising a sequence selected from the group consisting of SEQ Identifiers: B1566-B1574 of Table 3B. In some embodiments, the PEgRNA comprises a PBS comprising a sequence selected from the group consisting of SEQ ID NOs. 2431-2437 of Table 3B. In some embodiments, the PEgRNA comprises a PBS comprising a sequence selected from the group consisting of sequences GAACTATA and GAACTATAT as disclosed in Table 3B. In some embodiments, the PEgRNA comprises an editing template comprising a sequence selected from the group consisting of SEQ Identifiers: C9227-C9234 of Table 3C. In some embodiments, the PEgRNA comprises an editing template comprising a sequence selected from the group consisting of SEQ ID NOs: 13253-13260 of Table 3C. In some embodiments, the prime editing composition further comprises a ngRNA that comprises a spacer comprising a sequence selected from the group consisting of SEQ Identifiers: D915-D928 of Table 3D. In some embodiments, the prime editing composition further comprises a ngRNA that comprises a spacer comprising a sequence selected from the group consisting of SEQ ID NOs. 20548-20561 of Table 3D.

In some embodiments, the PEgRNA comprises a spacer comprising a sequence selected from the group consisting of SEQ Identifiers: A421-A422 of Table 3A. In some embodiments, the PEgRNA comprises a spacer comprising a sequence as set forth in SEQ ID NOs. 769-770 of Table 3A. In some embodiments, the PEgRNA comprises a PBS comprising a sequence selected from the group consisting of SEQ Identifiers: B1575-B1583 of Table 3B. In some embodiments, the PEgRNA comprises a PBS comprising a sequence selected from the group consisting of SEQ ID NOs. 2438-2444 of Table 3B. In some embodiments, the PEgRNA comprises a PBS comprising a sequence selected from the group consisting of sequences AGAACTAT and AGAACTATA as disclosed in Table 3B. In some embodiments, the PEgRNA comprises an editing template comprising a sequence selected from the group consisting of SEQ Identifiers: C9235-C9239 of Table 3C. In some embodiments, the PEgRNA comprises an editing template comprising a sequence selected from the group consisting of SEQ ID NOs: 13261-13265 of Table 3C. In some embodiments, the prime editing composition further comprises a ngRNA that comprises a spacer comprising a sequence selected from the group consisting of SEQ Identifiers: D930-D944, D946-D952, D1016 of Table 3D. In some embodiments, the prime editing composition further comprises a ngRNA that comprises a spacer comprising a sequence selected from the group consisting of SEQ ID NOs. 20563-20577, 20579-20585, 20649 of Table 3D.

In some embodiments, the PEgRNA comprises a spacer comprising a sequence selected from the group consisting of SEQ Identifiers: A481-A482 of Table 3A. In some embodiments, the PEgRNA comprises a spacer comprising a sequence as set forth in SEQ ID NOs. 829-830 of Table 3A. In some embodiments, the PEgRNA comprises a PBS comprising a sequence selected from the group consisting of SEQ Identifiers: B1764-B1772 of Table 3B. In some embodiments, the PEgRNA comprises a PBS comprising a sequence selected from the group consisting of SEQ ID NOs. 2583-2591 of Table 3B. In some embodiments, the PEgRNA comprises a PBS comprising a sequence selected from the group consisting of sequences AGTGTGAT and AGTGTGATT as disclosed in Table 3B. In some embodiments, the PEgRNA comprises an editing template comprising a sequence selected from the group consisting of SEQ Identifiers: C9240-C9265 of Table 3C. In some embodiments, the PEgRNA comprises an editing template comprising a sequence selected from the group consisting of SEQ ID NOs: 13266-13291 of Table 3C. In some embodiments, the prime editing composition further comprises a ngRNA that comprises a spacer comprising a sequence selected from the group consisting of SEQ Identifiers: D930-D944, D1016-D1019 and D1096 of Table 3D. In some embodiments, the prime editing composition further comprises a ngRNA that comprises a spacer comprising a sequence selected from the group consisting of SEQ ID NOs. 0.20563-20577, 20649-20652, and 20729 of Table 3D.

In some embodiments, the PEgRNA comprises a spacer comprising a sequence selected from the group consisting of SEQ Identifiers: A425-A426 of Table 3A. In some embodiments, the PEgRNA comprises a spacer comprising a sequence as set forth in SEQ ID NOs. 773-774 of Table 3A. In some embodiments, the PEgRNA comprises a PBS comprising a sequence selected from the group consisting of SEQ Identifiers: B1773-B1781 of Table 3B. In some embodiments, the PEgRNA comprises a PBS comprising a sequence selected from the group consisting of SEQ ID NOs. 2592-2598 of Table 3B. In some embodiments, the PEgRNA comprises a PBS comprising a sequence selected from the group consisting of sequences CCAAGAAC and CCAAGAACT as disclosed in Table 3B. In some embodiments, the PEgRNA comprises an editing template comprising a sequence selected from the group consisting of SEQ Identifiers: C9266-C9273 of Table 3C. In some embodiments, the PEgRNA comprises an editing template comprising a sequence selected from the group consisting of SEQ ID NOs: 13292-13299 of Table 3C. In some embodiments, the prime editing composition further comprises a ngRNA that comprises a spacer comprising a sequence selected from the group consisting of SEQ Identifiers: D930-D944, and D1016-D1018 of Table 3D. In some embodiments, the prime editing composition further comprises a ngRNA that comprises a spacer comprising a sequence selected from the group consisting of SEQ ID NOs. 20563-20577, and 20649-20651 of Table 3D.

In some embodiments, the PEgRNA comprises a spacer comprising the sequence of SEQ Identifier: A427 of Table 3A. In some embodiments, the PEgRNA comprises a spacer comprising a sequence as set forth in SEQ ID NO. 775 of Table 3A. In some embodiments, the PEgRNA comprises a PBS comprising a sequence selected from the group consisting of SEQ Identifiers: B1782-B1790 of Table 3B. In some embodiments, the PEgRNA comprises a PBS comprising a sequence selected from the group consisting of SEQ ID NOs. 2599-2605 of Table 3B. In some embodiments, the PEgRNA comprises a PBS comprising a sequence selected from the group consisting of sequences TCCAAGAA and TCCAAGAAC as disclosed in Table 3B. In some embodiments, the PEgRNA comprises an editing template comprising a sequence selected from the group consisting of SEQ Identifiers: C9274-C9282 of Table 3C. In some embodiments, the PEgRNA comprises an editing template comprising a sequence selected from the group consisting of SEQ ID NOs: 13300-13308 of Table 3C. In some embodiments, the prime editing composition further comprises a ngRNA that comprises a spacer comprising a sequence selected from the group consisting of SEQ Identifiers: D930-D944, D1016, and D1019 of Table 3D. In some embodiments, the prime editing composition further comprises a ngRNA that comprises a spacer comprising a sequence selected from the group consisting of SEQ ID NOs. 20563-20577, 20649, and 20652 of Table 3D.

In some embodiments, the PEgRNA comprises a spacer comprising a sequence selected from the group consisting of SEQ Identifiers: A508-A509 of Table 3A. In some embodiments, the PEgRNA comprises a spacer comprising a sequence as set forth in SEQ ID NOs. 856-857 of Table 3A. In some embodiments, the PEgRNA comprises a PBS comprising a sequence selected from the group consisting of SEQ Identifiers: B1971-B1979 of Table 3B. In some embodiments, the PEgRNA comprises a PBS comprising a sequence selected from the group consisting of SEQ ID NOs. 2746-2752 of Table 3B. In some embodiments, the PEgRNA comprises a PBS comprising a sequence selected from the group consisting of sequences GACCTCCA and GACCTCCAC as disclosed in Table 3B. In some embodiments, the PEgRNA comprises an editing template comprising a sequence selected from the group consisting of SEQ Identifiers: C9283-C9344 of Table 3C. In some embodiments, the PEgRNA comprises an editing template comprising a sequence selected from the group consisting of SEQ ID NOs: 13309-13370 of Table 3C. In some embodiments, the prime editing composition further comprises a ngRNA that comprises a spacer comprising a sequence selected from the group consisting of SEQ Identifiers: D916-D928, D1056-D1057, and D1097-D1100 of Table 3D. In some embodiments, the prime editing composition further comprises a ngRNA that comprises a spacer comprising a sequence selected from the group consisting of SEQ ID NOs. 20549-20561, 20689-20690, and 20730-20743 of Table 3D.

In some embodiments, the PEgRNA comprises a spacer comprising the sequence of SEQ Identifier: A510 of Table 3A. In some embodiments, the PEgRNA comprises a spacer comprising a sequence as set forth in SEQ ID NO. 858 of Table 3A. In some embodiments, the PEgRNA comprises a PBS comprising a sequence selected from the group consisting of SEQ Identifiers: B1980-B1988 of Table 3B. In some embodiments, the PEgRNA comprises a PBS comprising a sequence selected from the group consisting of SEQ ID NOs. 2753-2759 of Table 3B. In some embodiments, the PEgRNA comprises a PBS comprising a sequence selected from the group consisting of sequences TGACCTCC and TGACCTCCA as disclosed in Table 3B. In some embodiments, the PEgRNA comprises an editing template comprising a sequence selected from the group consisting of SEQ Identifiers: C9345-C9375 of Table 3C. In some embodiments, the PEgRNA comprises an editing template comprising a sequence selected from the group consisting of SEQ ID NOs: 13371-13401 of Table 3C. In some embodiments, the prime editing composition further comprises a ngRNA that comprises a spacer comprising a sequence selected from the group consisting of SEQ Identifiers: D933-D944, D1016, D1019, D1096, D1101, and D1102 of Table 3D. In some embodiments, the prime editing composition further comprises a ngRNA that comprises a spacer comprising a sequence selected from the group consisting of SEQ ID NOs. 20566-20577, 20649, 20652, 20729, 20744, and 20745 of Table 3D.

In some embodiments, the PEgRNA comprises a spacer comprising a sequence selected from the group consisting of SEQ Identifiers: A428-A429 of Table 3A. In some embodiments, the PEgRNA comprises a spacer comprising a sequence as set forth in SEQ ID NOs. 776-777 of Table 3A. In some embodiments, the PEgRNA comprises a PBS comprising a sequence selected from the group consisting of SEQ Identifiers: B1791-B1799 of Table 3B. In some embodiments, the PEgRNA comprises a PBS comprising a sequence selected from the group consisting of SEQ ID NOs. 2606-2612 of Table 3B. In some embodiments, the PEgRNA comprises a PBS comprising a sequence selected from the group consisting of sequences ACTATATT and ACTATATTG as disclosed in Table 3B. In some embodiments, the PEgRNA comprises an editing template comprising a sequence selected from the group consisting of SEQ Identifiers: C9376-C9377 of Table 3C. In some embodiments, the PEgRNA comprises an editing template comprising a sequence selected from the group consisting of SEQ ID NOs: 13402-13403 of Table 3C. In some embodiments, the prime editing composition further comprises a ngRNA that comprises a spacer comprising a sequence selected from the group consisting of SEQ Identifiers: D930-D944, D1016, D1017, and D1018 of Table 3D. In some embodiments, the prime editing composition further comprises a ngRNA that comprises a spacer comprising a sequence selected from the group consisting of SEQ ID NOs. 20563-20577, 20649, 20650, and 20651 of Table 3D.

In some embodiments, the PEgRNA comprises a spacer comprising a sequence selected from the group consisting of SEQ Identifiers: A465-A467 of Table 3A. In some embodiments, the PEgRNA comprises a spacer comprising a sequence as set forth in SEQ ID NOs. 813-815 of Table 3A. In some embodiments, the PEgRNA comprises a PBS comprising a sequence selected from the group consisting of SEQ Identifiers: B1800-B1808 of Table 3B. In some embodiments, the PEgRNA comprises a PBS comprising a sequence selected from the group consisting of SEQ ID NOs. 2613-2619 of Table 3B. In some embodiments, the PEgRNA comprises a PBS comprising a sequence selected from the group consisting of sequences GTGTGATT and GTGTGATTC as disclosed in Table 3B. In some embodiments, the PEgRNA comprises an editing template comprising a sequence selected from the group consisting of SEQ Identifiers: C9378-C9402 of Table 3C. In some embodiments, the PEgRNA comprises an editing template comprising a sequence selected from the group consisting of SEQ ID NOs: 13404-13428 of Table 3C. In some embodiments, the prime editing composition further comprises a ngRNA that comprises a spacer comprising a sequence selected from the group consisting of SEQ Identifiers: D922, and D1021-D1025 of Table 3D. In some embodiments, the prime editing composition further comprises a ngRNA that comprises a spacer comprising a sequence selected from the group consisting of SEQ ID NOs. 20555, and 20654-20658 of Table 3D.

In some embodiments, the PEgRNA comprises a spacer comprising a sequence selected from the group consisting of SEQ Identifiers: A433-A435 of Table 3A. In some embodiments, the PEgRNA comprises a spacer comprising a sequence as set forth in SEQ ID NOs. 781-783 of Table 3A. In some embodiments, the PEgRNA comprises a PBS comprising a sequence selected from the group consisting of SEQ Identifiers: B1809-B1817 of Table 3B. In some embodiments, the PEgRNA comprises a PBS comprising a sequence selected from the group consisting of SEQ ID NOs. 2620-2626 of Table 3B. In some embodiments, the PEgRNA comprises a PBS comprising a sequence selected from the group consisting of sequences CTATATTG and CTATATTGT as disclosed in Table 3B. In some embodiments, the PEgRNA comprises an editing template comprising the sequence of SEQ Identifier: C9403 of Table 3C. In some embodiments, the PEgRNA comprises an editing template comprising a sequence selected from the group consisting of SEQ ID NOs: 13429 of Table 3C. In some embodiments, the prime editing composition further comprises a ngRNA that comprises a spacer comprising a sequence selected from the group consisting of SEQ Identifiers: D922, and D1021-D1025 of Table 3D. In some embodiments, the prime editing composition further comprises a ngRNA that comprises a spacer comprising a sequence selected from the group consisting of SEQ ID NOs. 20555, and 20654-20658 of Table 3D.

In some embodiments, the PEgRNA comprises a spacer comprising a sequence selected from the group consisting of SEQ Identifiers: A501-A502 of Table 3A. In some embodiments, the PEgRNA comprises a spacer comprising a sequence as set forth in SEQ ID NOs. 849-850 of Table 3A. In some embodiments, the PEgRNA comprises a PBS comprising a sequence selected from the group consisting of SEQ Identifiers: B1818-B1826 of Table 3B. In some embodiments, the PEgRNA comprises a PBS comprising a sequence selected from the group consisting of SEQ ID NOs. 2627-2633 of Table 3B. In some embodiments, the PEgRNA comprises a PBS comprising a sequence selected from the group consisting of sequences CAGTGTGA and CAGTGTGAT as disclosed in Table 3B. In some embodiments, the PEgRNA comprises an editing template comprising a sequence selected from the group consisting of SEQ Identifiers: C9404-C9430 of Table 3C. In some embodiments, the PEgRNA comprises an editing template comprising a sequence selected from the group consisting of SEQ ID NOs: 13430-13456 of Table 3C. In some embodiments, the prime editing composition further comprises a ngRNA that comprises a spacer comprising a sequence selected from the group consisting of SEQ Identifiers: D930-D944, D946-D952, D1016, D1019, D1026, D1096, and D1103 of Table 3D. In some embodiments, the prime editing composition further comprises a ngRNA that comprises a spacer comprising a sequence selected from the group consisting of SEQ ID NOs. 20579-20585, 20649, 20652, 20659, and 20736 of Table 3D.

In some embodiments, the PEgRNA comprises a spacer comprising a sequence selected from the group consisting of SEQ Identifiers: A438-A441 of Table 3A. In some embodiments, the PEgRNA comprises a spacer comprising a sequence as set forth in SEQ ID NOs. 786-789 of Table 3A. In some embodiments, the PEgRNA comprises a PBS comprising a sequence selected from the group consisting of SEQ Identifiers: B1827-B1835 of Table 3B. In some embodiments, the PEgRNA comprises a PBS comprising a sequence selected from the group consisting of SEQ ID NOs. 2634-2640 of Table 3B. In some embodiments, the PEgRNA comprises a PBS comprising a sequence selected from the group consisting of sequences TCTCCAAG and TCTCCAAGA as disclosed in Table 3B. In some embodiments, the PEgRNA comprises an editing template comprising a sequence selected from the group consisting of SEQ Identifiers: C9431-C9441 of Table 3C. In some embodiments, the PEgRNA comprises an editing template comprising a sequence selected from the group consisting of SEQ ID NOs: 13457-13467 of Table 3C. In some embodiments, the prime editing composition further comprises a ngRNA that comprises a spacer comprising a sequence selected from the group consisting of SEQ Identifiers: D925, D930-D944, D1016-D1019, and D1028-D1032 of Table 3D. In some embodiments, the prime editing composition further comprises a ngRNA that comprises a spacer comprising a sequence selected from the group consisting of SEQ ID NOs. 20558, 20563-20577, 20649-20652, and 20661-20665 of Table 3D.

In some embodiments, the PEgRNA comprises a spacer comprising the sequence of SEQ Identifier: A442 of Table 3A. In some embodiments, the PEgRNA comprises a spacer comprising a sequence as set forth in SEQ ID NO. 790 of Table 3A. In some embodiments, the PEgRNA comprises a PBS comprising a sequence selected from the group consisting of SEQ Identifiers: B1836-B1844 of Table 3B. In some embodiments, the PEgRNA comprises a PBS comprising a sequence selected from the group consisting of SEQ ID NOs. 2641-2647 of Table 3B. In some embodiments, the PEgRNA comprises a PBS comprising a sequence selected from the group consisting of sequences TGAATAAC and TGAATAACT as disclosed in Table 3B. In some embodiments, the PEgRNA comprises an editing template comprising a sequence selected from the group consisting of SEQ Identifiers: C9442-C9457 of Table 3C. In some embodiments, the PEgRNA comprises an editing template comprising a sequence selected from the group consisting of SEQ ID NOs: 13468-13483 of Table 3C. In some embodiments, the prime editing composition further comprises a ngRNA that comprises a spacer comprising a sequence selected from the group consisting of SEQ Identifiers: D972-D981, D1002, D1033, D1034, D1035, D1036, D1037, D1055, D1073, D1104, and D1105 of Table 3D. In some embodiments, the prime editing composition further comprises a ngRNA that comprises a spacer comprising a sequence selected from the group consisting of SEQ ID NOs. 20605-20614, 20635, 20666, 20667, 20668, 20669, 20670, 20688, 20706, 20737, and 20738 of Table 3D.

In some embodiments, the PEgRNA comprises a spacer comprising a sequence selected from the group consisting of SEQ Identifiers: A443-A445 of Table 3A. In some embodiments, the PEgRNA comprises a spacer comprising a sequence as set forth in SEQ ID NOs. 791-793 of Table 3A. In some embodiments, the PEgRNA comprises a PBS comprising a sequence selected from the group consisting of SEQ Identifiers: B1845-B1853 of Table 3B. In some embodiments, the PEgRNA comprises a PBS comprising a sequence selected from the group consisting of SEQ ID NOs. 2648-2654 of Table 3B. In some embodiments, the PEgRNA comprises a PBS comprising a sequence selected from the group consisting of sequences CTCCAAGA and CTCCAAGAA as disclosed in Table 3B. In some embodiments, the PEgRNA comprises an editing template comprising a sequence selected from the group consisting of SEQ Identifiers: C9458-C9467 of Table 3C. In some embodiments, the PEgRNA comprises an editing template comprising a sequence selected from the group consisting of SEQ ID NOs: 13484-13493 of Table 3C. In some embodiments, the prime editing composition further comprises a ngRNA that comprises a spacer comprising a sequence selected from the group consisting of SEQ Identifiers: D922, and D1021-D1025 of Table 3D. In some embodiments, the prime editing composition further comprises a ngRNA that comprises a spacer comprising a sequence selected from the group consisting of SEQ ID NOs. 20555, and 20654-20658 of Table 3D.

In some embodiments, the PEgRNA comprises a spacer comprising a sequence selected from the group consisting of SEQ Identifiers: A511-A512 of Table 3A. In some embodiments, the PEgRNA comprises a spacer comprising a sequence as set forth in SEQ ID NOs. 859-860 of Table 3A. In some embodiments, the PEgRNA comprises a PBS comprising a sequence selected from the group consisting of SEQ Identifiers: B1989-B1997 of Table 3B. In some embodiments, the PEgRNA comprises a PBS comprising a sequence selected from the group consisting of SEQ ID NOs. 2760-2766 of Table 3B. In some embodiments, the PEgRNA comprises a PBS comprising a sequence selected from the group consisting of sequences ACCTCCAC and ACCTCCACT as disclosed in Table 3B. In some embodiments, the PEgRNA comprises an editing template comprising a sequence selected from the group consisting of SEQ Identifiers: C9468-C9498 of Table 3C. In some embodiments, the PEgRNA comprises an editing template comprising a sequence selected from the group consisting of SEQ ID NOs: 13494-13524 of Table 3C. In some embodiments, the prime editing composition further comprises a ngRNA that comprises a spacer comprising a sequence selected from the group consisting of SEQ Identifiers: D933-D944, D947-D952, D1016, D1019, D1026, D1096, D1101, D1102, and D1103 of Table 3D. In some embodiments, the prime editing composition further comprises a ngRNA that comprises a spacer comprising a sequence selected from the group consisting of SEQ ID NOs. 20566-20577, 20580-20585, 20649, 20652, 20659, 20729, 20734, 20735, and 20736 of Table 3D.

In some embodiments, the PEgRNA comprises a spacer comprising the sequence of SEQ Identifier: A446 of Table 3A. In some embodiments, the PEgRNA comprises a spacer comprising a sequence as set forth in SEQ ID NO. 794 of Table 3A. In some embodiments, the PEgRNA comprises a PBS comprising a sequence selected from the group consisting of SEQ Identifiers: B1854-B1862 of Table 3B. In some embodiments, the PEgRNA comprises a PBS comprising a sequence selected from the group consisting of SEQ ID NOs. 2655-2661 of Table 3B. In some embodiments, the PEgRNA comprises a PBS comprising a sequence selected from the group consisting of sequences ATTTCTTT and ATTTCTTTA as disclosed in Table 3B. In some embodiments, the PEgRNA comprises an editing template comprising a sequence selected from the group consisting of SEQ Identifiers: C9499-C9529 of Table 3C. In some embodiments, the PEgRNA comprises an editing template comprising a sequence selected from the group consisting of SEQ ID NOs: 13525-13555 of Table 3C. In some embodiments, the prime editing composition further comprises a ngRNA that comprises a spacer comprising a sequence selected from the group consisting of SEQ Identifiers: D969-D981, D1002, D1033, D1034, D1035, and D1106 of Table 3D. In some embodiments, the prime editing composition further comprises a ngRNA that comprises a spacer comprising a sequence selected from the group consisting of SEQ ID NOs. 20602-20614, 20635, 20666, 20667, 20668, and 20739 of Table 3D.

In some embodiments, the PEgRNA comprises a spacer comprising a sequence selected from the group consisting of SEQ Identifiers: A391-A392 of Table 3A. In some embodiments, the PEgRNA comprises a spacer comprising a sequence as set forth in SEQ ID NOs. 739-740 of Table 3A. In some embodiments, the PEgRNA comprises a PBS comprising a sequence selected from the group consisting of SEQ Identifiers: B1611-B1619 of Table 3B. In some embodiments, the PEgRNA comprises a PBS comprising a sequence selected from the group consisting of SEQ ID NOs. 2466-2472 of Table 3B. In some embodiments, the PEgRNA comprises a PBS comprising a sequence selected from the group consisting of sequences AACTATAT and AACTATATT as disclosed in Table 3B. In some embodiments, the PEgRNA comprises an editing template comprising a sequence selected from the group consisting of SEQ Identifiers: C9530-C9532 of Table 3C. In some embodiments, the PEgRNA comprises an editing template comprising a sequence selected from the group consisting of SEQ ID NOs: 13556-13558 of Table 3C. In some embodiments, the prime editing composition further comprises a ngRNA that comprises a spacer comprising a sequence selected from the group consisting of SEQ Identifiers: D930-D944, D946-D952, and D1016 of Table 3D. In some embodiments, the prime editing composition further comprises a ngRNA that comprises a spacer comprising a sequence selected from the group consisting of SEQ ID NOs. 20563-20577, 20579-20585, and 20649 of Table 3D.

In some embodiments, the PEgRNA comprises a spacer comprising a sequence selected from the group consisting of SEQ Identifiers: A447-A450 of Table 3A. In some embodiments, the PEgRNA comprises a spacer comprising a sequence as set forth in SEQ ID NOs. 795-798 of Table 3A. In some embodiments, the PEgRNA comprises a PBS comprising a sequence selected from the group consisting of SEQ Identifiers: B1863-B1871 of Table 3B. In some embodiments, the PEgRNA comprises a PBS comprising a sequence selected from the group consisting of SEQ ID NOs. 2662-2668 of Table 3B. In some embodiments, the PEgRNA comprises a PBS comprising a sequence selected from the group consisting of sequences CAAGAACT and CAAGAACTA as disclosed in Table 3B. In some embodiments, the PEgRNA comprises an editing template comprising a sequence selected from the group consisting of SEQ Identifiers: C9533-C9546 of Table 3C. In some embodiments, the PEgRNA comprises an editing template comprising a sequence selected from the group consisting of SEQ ID NOs: 13559-13572 of Table 3C. In some embodiments, the prime editing composition further comprises a ngRNA that comprises a spacer comprising a sequence selected from the group consisting of SEQ Identifiers: D915-D928, and D1021-D1025 of Table 3D. In some embodiments, the prime editing composition further comprises a ngRNA that comprises a spacer comprising a sequence selected from the group consisting of SEQ ID NOs. 20558-20671, and 20654-20658 of Table 3D.

In some embodiments, the PEgRNA comprises a spacer comprising a sequence selected from the group consisting of SEQ Identifiers: A489-A490 of Table 3A. In some embodiments, the PEgRNA comprises a spacer comprising a sequence as set forth in SEQ ID NOs. 837-838 of Table 3A. In some embodiments, the PEgRNA comprises a PBS comprising a sequence selected from the group consisting of SEQ Identifiers: B1944-B1952 of Table 3B. In some embodiments, the PEgRNA comprises a PBS comprising a sequence selected from the group consisting of SEQ ID NOs. 2725-2731 of Table 3B. In some embodiments, the PEgRNA comprises a PBS comprising a sequence selected from the group consisting of sequences GTGATTCC and GTGATTCCA as disclosed in Table 3B. In some embodiments, the PEgRNA comprises an editing template comprising a sequence selected from the group consisting of SEQ Identifiers: C9547-C9592 of Table 3C. In some embodiments, the PEgRNA comprises an editing template comprising a sequence selected from the group consisting of SEQ ID NOs: 13573-13618 of Table 3C. In some embodiments, the prime editing composition further comprises a ngRNA that comprises a spacer comprising a sequence selected from the group consisting of SEQ Identifiers: D915-D928, D1056, and D1057 of Table 3D. In some embodiments, the prime editing composition further comprises a ngRNA that comprises a spacer comprising a sequence selected from the group consisting of SEQ ID NOs. 20558-20671, 20689, and 20690 of Table 3D.

In some embodiments, the PEgRNA comprises a spacer comprising the sequence of SEQ Identifier: A451 of Table 3A. In some embodiments, the PEgRNA comprises a spacer comprising a sequence as set forth in SEQ ID NO. 799 of Table 3A. In some embodiments, the PEgRNA comprises a PBS comprising a sequence selected from the group consisting of SEQ Identifiers: B1872-B1880 of Table 3B. In some embodiments, the PEgRNA comprises a PBS comprising a sequence selected from the group consisting of SEQ ID NOs. 2669-2675 of Table 3B. In some embodiments, the PEgRNA comprises a PBS comprising a sequence selected from the group consisting of sequences TGTGATTC and TGTGATTCC as disclosed in Table 3B. In some embodiments, the PEgRNA comprises an editing template comprising a sequence selected from the group consisting of SEQ Identifiers: C9593-C9616 of Table 3C. In some embodiments, the PEgRNA comprises an editing template comprising a sequence selected from the group consisting of SEQ ID NOs: 13619-13642 of Table 3C. In some embodiments, the prime editing composition further comprises a ngRNA that comprises a spacer comprising a sequence selected from the group consisting of SEQ Identifiers: D930-D944, D1016, and D1019 of Table 3D. In some embodiments, the prime editing composition further comprises a ngRNA that comprises a spacer comprising a sequence selected from the group consisting of SEQ ID NOs. 20563-20577, 20649, and 20652 of Table 3D.

In some embodiments, the PEgRNA comprises a spacer comprising a sequence selected from the group consisting of SEQ Identifiers: A456 and A470 of Table 3A. In some embodiments, the PEgRNA comprises a spacer comprising a sequence as set forth in SEQ ID NOs. 804 and 818 of Table 3A. In some embodiments, the PEgRNA comprises a PBS comprising a sequence selected from the group consisting of SEQ Identifiers: B1899-B1907 of Table 3B. In some embodiments, the PEgRNA comprises a PBS comprising a sequence selected from the group consisting of SEQ ID NOs. 2690-2696 of Table 3B. In some embodiments, the PEgRNA comprises a PBS comprising a sequence selected from the group consisting of sequences TGATTCCA and TGATTCCAC as disclosed in Table 3B. In some embodiments, the PEgRNA comprises an editing template comprising a sequence selected from the group consisting of SEQ Identifiers: C9617-C9638 of Table 3C. In some embodiments, the PEgRNA comprises an editing template comprising a sequence selected from the group consisting of SEQ ID NOs: 13643-13664 of Table 3C. In some embodiments, the prime editing composition further comprises a ngRNA that comprises a spacer comprising a sequence selected from the group consisting of SEQ Identifiers: D930-D952 D1016, D1019, and D1026 of Table 3D. In some embodiments, the prime editing composition further comprises a ngRNA that comprises a spacer comprising a sequence selected from the group consisting of SEQ ID NOs. 20563-20585, 20649, 20652, and 20659 of Table 3D.

In some embodiments, the PEgRNA comprises a spacer comprising a sequence selected from the group consisting of SEQ Identifiers: A506, A507, A513, and A514 of Table 3A. In some embodiments, the PEgRNA comprises a spacer comprising a sequence as set forth in SEQ ID NOs. 854-855 and 861-862 of Table 3A. In some embodiments, the PEgRNA comprises a PBS comprising a sequence selected from the group consisting of SEQ Identifiers: B1926-B1934 of Table 3B. In some embodiments, the PEgRNA comprises a PBS comprising a sequence selected from the group consisting of SEQ ID NOs. 2711-2717 of Table 3B. In some embodiments, the PEgRNA comprises a PBS comprising a sequence selected from the group consisting of sequences TCAGTGTG and TCAGTGTGA as disclosed in Table 3B. In some embodiments, the PEgRNA comprises an editing template comprising a sequence selected from the group consisting of SEQ Identifiers: C9639-C9694 of Table 3C. In some embodiments, the PEgRNA comprises an editing template comprising a sequence selected from the group consisting of SEQ ID NOs: 13665-13720 of Table 3C. In some embodiments, the prime editing composition further comprises a ngRNA that comprises a spacer comprising a sequence selected from the group consisting of SEQ Identifiers: D915-D928, D1021-D1025, D1056, and D1057 of Table 3D. In some embodiments, the prime editing composition further comprises a ngRNA that comprises a spacer comprising a sequence selected from the group consisting of SEQ ID NOs. 20558-20671, 20654-20658, 20689, and 20690 of Table 3D.

In some embodiments, the PEgRNA comprises a spacer comprising a sequence selected from the group consisting of SEQ Identifiers: A503-A504 of Table 3A. In some embodiments, the PEgRNA comprises a spacer comprising a sequence as set forth in SEQ ID NOs. 851-852 of Table 3A. In some embodiments, the PEgRNA comprises a PBS comprising a sequence selected from the group consisting of SEQ Identifiers: B1953-B1961 of Table 3B. In some embodiments, the PEgRNA comprises a PBS comprising a sequence selected from the group consisting of SEQ ID NOs. 2732-2738 of Table 3B. In some embodiments, the PEgRNA comprises a PBS comprising a sequence selected from the group consisting of sequences CTCAGTGT amd CTCAGTGTG as disclosed in Table 3B. In some embodiments, the PEgRNA comprises an editing template comprising a sequence selected from the group consisting of SEQ Identifiers: C9695-C9723 of Table 3C. In some embodiments, the PEgRNA comprises an editing template comprising a sequence selected from the group consisting of SEQ ID NOs: 13721-13749 of Table 3C. In some embodiments, the prime editing composition further comprises a ngRNA that comprises a spacer comprising a sequence selected from the group consisting of SEQ Identifiers: D930-D944, D1016-D1019, D1096, and D1101 of Table 3D. In some embodiments, the prime editing composition further comprises a ngRNA that comprises a spacer comprising a sequence selected from the group consisting of SEQ ID NOs. 20563-20577, 20649-20652, 20729, and 20734 of Table 3D.

In some embodiments, the PEgRNA comprises a spacer comprising the sequence of SEQ Identifier: A505 of Table 3A. In some embodiments, the PEgRNA comprises a spacer comprising a sequence as set forth in SEQ ID NO. 853 of Table 3A. In some embodiments, the PEgRNA comprises a PBS comprising a sequence selected from the group consisting of SEQ Identifiers: B1962-B1970 of Table 3B. In some embodiments, the PEgRNA comprises a PBS comprising a sequence selected from the group consisting of SEQ ID NOs. 2739-2745 of Table 3B. In some embodiments, the PEgRNA comprises a PBS comprising a sequence selected from the group consisting of sequences ACTCAGTG and ACTCAGTGT as disclosed in Table 3B. In some embodiments, the PEgRNA comprises an editing template comprising a sequence selected from the group consisting of SEQ Identifiers: C9724-C9753 of Table 3C. In some embodiments, the PEgRNA comprises an editing template comprising a sequence selected from the group consisting of SEQ ID NOs: 13750-13779 of Table 3C. In some embodiments, the prime editing composition further comprises a ngRNA that comprises a spacer comprising a sequence selected from the group consisting of SEQ Identifiers: D930-D944, D1016, D1019, D1096, D1101, and D1102 of Table 3D. In some embodiments, the prime editing composition further comprises a ngRNA that comprises a spacer comprising a sequence selected from the group consisting of SEQ ID NOs. 20563-20577, 20649, 20652, 20729, 20734, 20735, and 20736 of Table 3D.

In some embodiments, the PEgRNA comprises a spacer comprising a sequence selected from the group consisting of SEQ Identifiers: A515-A516 of Table 3A. In some embodiments, the PEgRNA comprises a spacer comprising a sequence as set forth in SEQ ID NOs. 863-864 of Table 3A. In some embodiments, the PEgRNA comprises a PBS comprising a sequence selected from the group consisting of SEQ Identifiers: B1656-B1664 of Table 3B. In some embodiments, the PEgRNA comprises a PBS comprising a sequence selected from the group consisting of SEQ ID NOs. 3173-3179 of Table 3B. In some embodiments, the PEgRNA comprises a PBS comprising a sequence selected from the group consisting of sequences AGCAAGAA and AGCAAGAAT as disclosed in Table 3B. In some embodiments, the PEgRNA comprises an editing template comprising a sequence selected from the group consisting of SEQ Identifiers: C9754-C9784 of Table 3C. In some embodiments, the PEgRNA comprises an editing template comprising a sequence selected from the group consisting of SEQ ID NOs: 13780-13810 of Table 3C. In some embodiments, the prime editing composition further comprises a ngRNA that comprises a spacer comprising a sequence selected from the group consisting of SEQ Identifiers: D959-D981, D1002, D1033, D1034, D1035, D1040, D1041, D1106, D1107, and D1108 of Table 3D. In some embodiments, the prime editing composition further comprises a ngRNA that comprises a spacer comprising a sequence selected from the group consisting of SEQ ID NOs. 20592-20614, 20635, 20666, 20667, 20668, 20673, 20674, 20739, 20740, and 20741 of Table 3D.

In some embodiments, the PEgRNA comprises a spacer comprising a sequence selected from the group consisting of SEQ Identifiers: A452-A453 of Table 3A. In some embodiments, the PEgRNA comprises a spacer comprising a sequence as set forth in SEQ ID NOs. 800-801 of Table 3A. In some embodiments, the PEgRNA comprises a PBS comprising a sequence selected from the group consisting of SEQ Identifiers: B1881-B1889 of Table 3B. In some embodiments, the PEgRNA comprises a PBS comprising a sequence selected from the group consisting of SEQ ID NOs. 2676-2682 of Table 3B. In some embodiments, the PEgRNA comprises a PBS comprising a sequence selected from the group consisting of sequences GCAAGGTG and GCAAGGTGA as disclosed in Table 3B. In some embodiments, the PEgRNA comprises an editing template comprising a sequence selected from the group consisting of SEQ Identifiers: C9785-C9806 of Table 3C. In some embodiments, the PEgRNA comprises an editing template comprising a sequence selected from the group consisting of SEQ ID NOs: 13811-13832 of Table 3C. In some embodiments, the prime editing composition further comprises a ngRNA that comprises a spacer comprising a sequence selected from the group consisting of SEQ Identifiers: D959-D981, D1002, D1033-D1037, D1040, D1041, D1055, and D1073 of Table 3D. In some embodiments, the prime editing composition further comprises a ngRNA that comprises a spacer comprising a sequence selected from the group consisting of SEQ ID NOs. 20592-20614, 20635, 20666-20670, 20673, 20674, 20688, and 20706 of Table 3D.

In some embodiments, the PEgRNA comprises a spacer comprising a sequence selected from the group consisting of SEQ Identifiers: A454-A455 of Table 3A. In some embodiments, the PEgRNA comprises a spacer comprising a sequence as set forth in SEQ ID NOs. 802-803 of Table 3A. In some embodiments, the PEgRNA comprises a PBS comprising a sequence selected from the group consisting of SEQ Identifiers: B1890-B1898 of Table 3B. In some embodiments, the PEgRNA comprises a PBS comprising a sequence selected from the group consisting of SEQ ID NOs. 2682-2689 of Table 3B. In some embodiments, the PEgRNA comprises a PBS comprising a sequence selected from the group consisting of sequences TTCTCCAA and TTCTCCAAG as disclosed in Table 3B. In some embodiments, the PEgRNA comprises an editing template comprising a sequence selected from the group consisting of SEQ Identifiers: C9807-C9818 of Table 3C. In some embodiments, the PEgRNA comprises an editing template comprising a sequence selected from the group consisting of SEQ ID NOs: 13833-13844 of Table 3C. In some embodiments, the prime editing composition further comprises a ngRNA that comprises a spacer comprising a sequence selected from the group consisting of SEQ Identifiers: D930-D944, D946-D952, D1016, D1019, and D1026 of Table 3D. In some embodiments, the prime editing composition further comprises a ngRNA that comprises a spacer comprising a sequence selected from the group consisting of SEQ ID NOs. 20563-20577, 20579-20585, 20649, 20652, and 20659 of Table 3D.

In some embodiments, the PEgRNA comprises a spacer comprising a sequence selected from the group consisting of SEQ Identifiers: A517-A518 of Table 3A. In some embodiments, the PEgRNA comprises a spacer comprising a sequence as set forth in SEQ ID NOs. 865-866 of Table 3A. In some embodiments, the PEgRNA comprises a PBS comprising a sequence selected from the group consisting of SEQ Identifiers: B1998-B2006 of Table 3B. In some embodiments, the PEgRNA comprises a PBS comprising a sequence selected from the group consisting of SEQ ID NOs. 2767-2773 of Table 3B. In some embodiments, the PEgRNA comprises a PBS comprising a sequence selected from the group consisting of sequences ATTGGTCT and ATTGGTCTA as disclosed in Table 3B. In some embodiments, the PEgRNA comprises an editing template comprising a sequence selected from the group consisting of SEQ Identifiers: C9819-C9824 of Table 3C. In some embodiments, the PEgRNA comprises an editing template comprising a sequence selected from the group consisting of SEQ ID NOs: 13845-13850 of Table 3C. In some embodiments, the prime editing composition further comprises a ngRNA that comprises a spacer comprising a sequence selected from the group consisting of SEQ Identifiers: D990-D992, D1000, D1001, and D1109-D1112 of Table 3D. In some embodiments, the prime editing composition further comprises a ngRNA that comprises a spacer comprising a sequence selected from the group consisting of SEQ ID NOs. 20623-20625, 20633, 20634, 20642-20645 of Table 3D.

In some embodiments, the PEgRNA comprises a spacer comprising the sequence of SEQ Identifier: A519 of Table 3A. In some embodiments, the PEgRNA comprises a spacer comprising a sequence as set forth in SEQ ID NO. 867 of Table 3A. In some embodiments, the PEgRNA comprises a PBS comprising a sequence selected from the group consisting of SEQ Identifiers: B2007-B2015 of Table 3B. In some embodiments, the PEgRNA comprises a PBS comprising a sequence selected from the group consisting of SEQ ID NOs. 2774-2780 of Table 3B. In some embodiments, the PEgRNA comprises a PBS comprising a sequence selected from the group consisting of sequences TATTGGTC and TATTGGTCT as disclosed in Table 3B. In some embodiments, the PEgRNA comprises an editing template comprising a sequence selected from the group consisting of SEQ Identifiers: C9825-C9828 of Table 3C. In some embodiments, the PEgRNA comprises an editing template comprising a sequence selected from the group consisting of SEQ ID NOs: 13851-13854 of Table 3C. In some embodiments, the prime editing composition further comprises a ngRNA that comprises a spacer comprising a sequence selected from the group consisting of SEQ Identifiers: D973-D981, D1002, D1033-D1037, D1055, D1073, D1104, and D1105 of Table 3D. In some embodiments, the prime editing composition further comprises a ngRNA that comprises a spacer comprising a sequence selected from the group consisting of SEQ ID NOs. 20606-20614, 20635, 20666-20670, 20688, 20706, 20737, and 20738 of Table 3D.

In some embodiments, the PEgRNA comprises a spacer comprising a sequence selected from the group consisting of SEQ Identifiers: A457-A458 of Table 3A. In some embodiments, the PEgRNA comprises a spacer comprising a sequence as set forth in SEQ ID NOs. 805-806 of Table 3A. In some embodiments, the PEgRNA comprises a PBS comprising a sequence selected from the group consisting of SEQ Identifiers: B1908-B1916 of Table 3B. In some embodiments, the PEgRNA comprises a PBS comprising a sequence selected from the group consisting of SEQ ID NOs. 2697-2703 of Table 3B. In some embodiments, the PEgRNA comprises a PBS comprising a sequence selected from the group consisting of sequences CAAGGTGA and CAAGGTGAA as disclosed in Table 3B. In some embodiments, the PEgRNA comprises an editing template comprising a sequence selected from the group consisting of SEQ Identifiers: C9829-C9870 of Table 3C. In some embodiments, the PEgRNA comprises an editing template comprising a sequence selected from the group consisting of SEQ ID NOs: 13855-13896 of Table 3C. In some embodiments, the prime editing composition further comprises a ngRNA that comprises a spacer comprising a sequence selected from the group consisting of SEQ Identifiers: D990-D992 and D999-D1001 of Table 3D. In some embodiments, the prime editing composition further comprises a ngRNA that comprises a spacer comprising a sequence selected from the group consisting of SEQ ID NOs. 20623-20625, and 20632-20634 of Table 3D.

In some embodiments, the PEgRNA comprises a spacer comprising a sequence selected from the group consisting of SEQ Identifiers: A478-A480 of Table 3A. In some embodiments, the PEgRNA comprises a spacer comprising a sequence as set forth in SEQ ID NOs. 826-828 of Table 3A. In some embodiments, the PEgRNA comprises a PBS comprising a sequence selected from the group consisting of SEQ Identifiers: B1935-B1943 of Table 3B. In some embodiments, the PEgRNA comprises a PBS comprising a sequence selected from the group consisting of SEQ ID NOs. 2718-2724 of Table 3B. In some embodiments, the PEgRNA comprises a PBS comprising a sequence selected from the group consisting of sequences GATTCCAC and GATTCCACC as disclosed in Table 3B. In some embodiments, the PEgRNA comprises an editing template comprising a sequence selected from the group consisting of SEQ Identifiers: C9871-C9891 of Table 3C. In some embodiments, the PEgRNA comprises an editing template comprising a sequence selected from the group consisting of SEQ ID NOs: 13897-13917 of Table 3C. In some embodiments, the prime editing composition further comprises a ngRNA that comprises a spacer comprising a sequence selected from the group consisting of SEQ Identifiers: D925, and D1028-D1032 of Table 3D. In some embodiments, the prime editing composition further comprises a ngRNA that comprises a spacer comprising a sequence selected from the group consisting of SEQ ID NOs. 20558, and 20661-20665 of Table 3D.

In some embodiments, the editing target sequence or the complementary sequence on the CFTR gene thereof comprises a mutation that encodes an amino acid substitution R560T, S557T, A559P, and/or A559E relative to a wild type CFTR polypeptide set forth in SEQ ID NO: 19, and wherein the editing template encodes an intended nucleotide edit that corrects the mutation. In some embodiments, the editing target sequence or the complementary sequence on the CFTR gene thereof comprises a G→→C mutation (c.1679G>C) at position 117,587,833, a T→→A mutation (c.1669T>A) at position 117,587,823, a deletion of C (c.1670delC) at position 117,587,824, a deletion of A (c.1674delA) at position 117,587,828, a G→→C mutation (c.1675G>C) at position 117,587,829, and/or a C→→A mutation (c.1676C>A) at position 117,587,830 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, the editing template comprises a wild type CFTR gene sequence. Accordingly, in some embodiments, the newly synthesized single stranded DNA encoded by the editing template is capable of correcting the c.1679G>C mutation, the c.1669T>A mutation, the c.1670delC mutation, the c.1674delA mutation, the c.1675G>C mutation, and/or the c.1676C>A mutation, in the target CFTR gene. In some embodiments, the newly synthesized single stranded DNA encoded by the editing template is capable of correcting all of the c.1679G>C mutation, the c.1669T>A mutation, the c.1670delC mutation, the c.1674delA mutation, the c.1675G>C mutation, and the c.1676C>A mutation in the target CFTR gene. In some embodiments, two or more of the c.1679G>C mutation, the c.1669T>A mutation, the c.1670delC mutation, the c.1674delA mutation, the c.1675G>C mutation, and the c.1676C>A mutation, are located on the same CFTR gene. In some embodiments, the newly synthesized single stranded DNA encoded by the editing template is capable of correcting the c.1679G>C mutation, the c.1669T>A mutation, the c.1670delC mutation, the c.1674delA mutation, the c.1675G>C mutation, and the c.1676C>A mutation that are located on different CFTR genes. For example, in some embodiments, a subject may comprise two copies of CFTR genes, each comprising a different mutation selected from the group consisting of the c.1679G>C mutation, the c.1669T>A mutation, the c.1670delC mutation, the c.1674delA mutation, the c.1675G>C mutation, and the c.1676C>A mutation the c.1679+5A>G mutation, all of which may be corrected by an editing template of a single PEgRNA. In some embodiments, the editing template is capable of correcting one or more, or all of the c.1679G>C mutation, the c.1669T>A mutation, the c.1670delC mutation, the c.1674delA mutation, the c.1675G>C mutation, the c.1676C>A mutation, the c.1679+1G>T mutation that are in different subjects, wherein each subject may have one or more of the mutations.

In some embodiments, the PEgRNA comprises a spacer comprising a sequence selected from the group consisting of SEQ Identifiers: A520-A521 of Table 3A. In some embodiments, the PEgRNA comprises a spacer comprising a sequence as set forth in SEQ ID NOs. 868-869 of Table 3A. In some embodiments, the PEgRNA comprises a PBS comprising a sequence selected from the group consisting of SEQ Identifiers: B2016-B2024 of Table 3B. In some embodiments, the PEgRNA comprises a PBS comprising a sequence selected from the group consisting of SEQ ID NOs. 2781-2787 of Table 3B. In some embodiments, the PEgRNA comprises a PBS comprising a sequence selected from the group consisting of sequences TGCTAAAG and TGCTAAAGA as disclosed in Table 3B. In some embodiments, the PEgRNA comprises an editing template comprising a sequence selected from the group consisting of SEQ Identifiers: C9892-C9922 of Table 3C. In some embodiments, the PEgRNA comprises an editing template comprising a sequence selected from the group consisting of SEQ ID NOs: 13918-13948 of Table 3C. In some embodiments, the prime editing composition further comprises a ngRNA that comprises a spacer comprising a sequence selected from the group consisting of SEQ Identifiers: D936-D952, D1016, D1019, D1026, and D1113-D1122 of Table 3D. In some embodiments, the prime editing composition further comprises a ngRNA that comprises a spacer comprising a sequence selected from the group consisting of SEQ ID NOs. 20569-20585, 20649, 20652, 20659, and 20746-20755 of Table 3D.

In some embodiments, the PEgRNA comprises a spacer comprising a sequence selected from the group consisting of SEQ Identifiers: A481-A482 of Table 3A. In some embodiments, the PEgRNA comprises a spacer comprising a sequence as set forth in SEQ ID NOs. 829-830 of Table 3A. In some embodiments, the PEgRNA comprises a PBS comprising a sequence selected from the group consisting of SEQ Identifiers: B1764-B1772 of Table 3B. In some embodiments, the PEgRNA comprises a PBS comprising a sequence selected from the group consisting of SEQ ID NOs. 2585-2591 of Table 3B. In some embodiments, the PEgRNA comprises a PBS comprising a sequence selected from the group consisting of sequences AGTGTGAT and AGTGTGATT as disclosed in Table 3B. In some embodiments, the PEgRNA comprises an editing template comprising a sequence selected from the group consisting of SEQ Identifiers: C9923-C9926 of Table 3C. In some embodiments, the PEgRNA comprises an editing template comprising a sequence selected from the group consisting of SEQ ID NOs: 13949-13952 of Table 3C. In some embodiments, the prime editing composition further comprises a ngRNA that comprises a spacer comprising a sequence selected from the group consisting of SEQ Identifiers: D933-D944, D1016, D1018, D1019, and D1116 of Table 3D. In some embodiments, the prime editing composition further comprises a ngRNA that comprises a spacer comprising a sequence selected from the group consisting of SEQ ID NOs. 20566-20577, 20649, 20651, 20652, and 20749 of Table 3D.

In some embodiments, the PEgRNA comprises a spacer comprising a sequence selected from the group consisting of SEQ Identifiers: A522-A523 of Table 3A. In some embodiments, the PEgRNA comprises a spacer comprising a sequence as set forth in SEQ ID NOs. 870-871 of Table 3A. In some embodiments, the PEgRNA comprises a PBS comprising a sequence selected from the group consisting of SEQ Identifiers: B1971-B1979 of Table 3B. In some embodiments, the PEgRNA comprises a PBS comprising a sequence selected from the group consisting of SEQ ID NOs. 2746-2752 of Table 3B. In some embodiments, the PEgRNA comprises a PBS comprising a sequence selected from the group consisting of sequences GACCTCCA and GACCTCCAC as disclosed in Table 3B. In some embodiments, the PEgRNA comprises an editing template comprising a sequence selected from the group consisting of SEQ Identifiers: C9927-C9956 of Table 3C. In some embodiments, the PEgRNA comprises an editing template comprising a sequence selected from the group consisting of SEQ ID NOs: 13953-13982 of Table 3C. In some embodiments, the prime editing composition further comprises a ngRNA that comprises a spacer comprising a sequence selected from the group consisting of SEQ Identifiers: D916-D928, D1056, D1057, D1097, D1099, D1123, and D1124 of Table 3D. In some embodiments, the prime editing composition further comprises a ngRNA that comprises a spacer comprising a sequence selected from the group consisting of SEQ ID NOs. 20549-20561, 20689, 20690, 20730, 20732, 20756, and 20757 of Table 3D.

In some embodiments, the PEgRNA comprises a spacer comprising the sequence of SEQ Identifier: A524 of Table 3A. In some embodiments, the PEgRNA comprises a spacer comprising a sequence as set forth in SEQ ID NO. 872 of Table 3A. In some embodiments, the PEgRNA comprises a PBS comprising a sequence selected from the group consisting of SEQ Identifiers: B1980-B1988 of Table 3B. In some embodiments, the PEgRNA comprises a PBS comprising a sequence selected from the group consisting of SEQ ID NOs. 2753-2759 of Table 3B. In some embodiments, the PEgRNA comprises a PBS comprising a sequence selected from the group consisting of sequences TGACCTCC and TGACCTCCA as disclosed in Table 3B. In some embodiments, the PEgRNA comprises an editing template comprising a sequence selected from the group consisting of SEQ Identifiers: C9957-C9972 of Table 3C. In some embodiments, the PEgRNA comprises an editing template comprising a sequence selected from the group consisting of SEQ ID NOs: 13983-13998 of Table 3C. In some embodiments, the prime editing composition further comprises a ngRNA that comprises a spacer comprising a sequence selected from the group consisting of SEQ Identifiers: D933-D944, D1016, D1019, D1116, D1117, and D1118 of Table 3D. In some embodiments, the prime editing composition further comprises a ngRNA that comprises a spacer comprising a sequence selected from the group consisting of SEQ ID NOs. 20566-20577, 20649, 20652, 20749, 20750, and 20751 of Table 3D.

In some embodiments, the PEgRNA comprises a spacer comprising a sequence selected from the group consisting of SEQ Identifier: A465-A467 of Table 3A. In some embodiments, the PEgRNA comprises a spacer comprising a sequence as set forth in SEQ ID NOs. 813-814 of Table 3A. In some embodiments, the PEgRNA comprises a PBS comprising a sequence selected from the group consisting of SEQ Identifiers: B1800-B1808 of Table 3B. In some embodiments, the PEgRNA comprises a PBS comprising a sequence selected from the group consisting of SEQ ID NOs. 2613-2619 of Table 3B. In some embodiments, the PEgRNA comprises a PBS comprising a sequence selected from the group consisting of sequences GTGTGATT and GTGTGATTC as disclosed in Table 3B. In some embodiments, the PEgRNA comprises an editing template comprising a sequence selected from the group consisting of SEQ Identifiers: C9973-C9975 of Table 3C. In some embodiments, the PEgRNA comprises an editing template comprising a sequence selected from the group consisting of SEQ ID NOs: 13999-14001 of Table 3C. In some embodiments, the prime editing composition further comprises a ngRNA that comprises a spacer comprising a sequence selected from the group consisting of SEQ Identifiers: D1021, D1023, and D1025 of Table 3D. In some embodiments, the prime editing composition further comprises a ngRNA that comprises a spacer comprising a sequence selected from the group consisting of SEQ ID NOs. 20654. 20656, and 20658 of Table 3D.

In some embodiments, the PEgRNA comprises a spacer comprising the sequence of SEQ Identifier: A525 of Table 3A. In some embodiments, the PEgRNA comprises a spacer comprising a sequence as set forth in SEQ ID NO. 873 of Table 3A. In some embodiments, the PEgRNA comprises a PBS comprising a sequence selected from the group consisting of SEQ Identifiers: B2025-B2033 of Table 3B. In some embodiments, the PEgRNA comprises a PBS comprising a sequence selected from the group consisting of SEQ ID NOs. 2788-2794 of Table 3B. In some embodiments, the PEgRNA comprises a PBS comprising a sequence selected from the group consisting of sequences CTAAAGAA and CTAAAGAAA as disclosed in Table 3B. In some embodiments, the PEgRNA comprises an editing template comprising a sequence selected from the group consisting of SEQ Identifiers: C9976-C10006 of Table 3C. In some embodiments, the PEgRNA comprises an editing template comprising a sequence selected from the group consisting of SEQ ID NOs: 14002-14032 of Table 3C. In some embodiments, the prime editing composition further comprises a ngRNA that comprises a spacer comprising a sequence selected from the group consisting of SEQ Identifiers: D930-D944, D1016, D1019, and D1116-D1122 of Table 3D. In some embodiments, the prime editing composition further comprises a ngRNA that comprises a spacer comprising a sequence selected from the group consisting of SEQ ID NOs. 20563-20577, 20649, 20652, and 20749-20755 of Table 3D.

In some embodiments, the PEgRNA comprises a spacer comprising a sequence selected from the group consisting of SEQ Identifiers: A501-A502. of Table 3A. In some embodiments, the PEgRNA comprises a spacer comprising a sequence as set forth in SEQ ID NOs. 849-850 of Table 3A. In some embodiments, the PEgRNA comprises a PBS comprising a sequence selected from the group consisting of SEQ Identifiers: B1818-B1826 of Table 3B. In some embodiments, the PEgRNA comprises a PBS comprising a sequence selected from the group consisting of SEQ ID NOs. 2627-2633 of Table 3B. In some embodiments, the PEgRNA comprises a PBS comprising a sequence selected from the group consisting of sequences CAGTGTGA and CAGTGTGAT as disclosed in Table 3B. In some embodiments, the PEgRNA comprises an editing template comprising a sequence selected from the group consisting of SEQ Identifiers: C10007-C10011 of Table 3C. In some embodiments, the PEgRNA comprises an editing template comprising a sequence selected from the group consisting of SEQ ID NOs: 14033-14037 of Table 3C. In some embodiments, the prime editing composition further comprises a ngRNA that comprises a spacer comprising a sequence selected from the group consisting of SEQ Identifiers: D933-D944, D947-D952, D1016, D1019, D1026, D1113, and D1116 of Table 3D. In some embodiments, the prime editing composition further comprises a ngRNA that comprises a spacer comprising a sequence selected from the group consisting of SEQ ID NOs. 20566-20577, 20580-20585, 20649, 20652, 20659, 20746 and 20749 of Table 3D.

In some embodiments, the PEgRNA comprises a spacer comprising a sequence selected from the group consisting of SEQ Identifiers: A526-A527 of Table 3A. In some embodiments, the PEgRNA comprises a spacer comprising a sequence as set forth in SEQ ID NOs. 874-875 of Table 3A. In some embodiments, the PEgRNA comprises a PBS comprising a sequence selected from the group consisting of SEQ Identifiers: B2034-B2042 of Table 3B. In some embodiments, the PEgRNA comprises a PBS comprising a sequence selected from the group consisting of SEQ ID NOs. 2795-2801 of Table 3B. In some embodiments, the PEgRNA comprises a PBS comprising a sequence selected from the group consisting of sequences AGCATTTG and AGCATTTGC as disclosed in Table 3B. In some embodiments, the PEgRNA comprises an editing template comprising a sequence selected from the group consisting of SEQ Identifiers: C10012-C10037 of Table 3C. In some embodiments, the PEgRNA comprises an editing template comprising a sequence selected from the group consisting of SEQ ID NOs: 14038-14063 of Table 3C. In some embodiments, the prime editing composition further comprises a ngRNA that comprises a spacer comprising a sequence selected from the group consisting of SEQ Identifiers: D992, D1001, and D1125-D1128 of Table 3D. In some embodiments, the prime editing composition further comprises a ngRNA that comprises a spacer comprising a sequence selected from the group consisting of SEQ ID NOs. 20625, 20634, and 20658-20661 of Table 3D.

In some embodiments, the PEgRNA comprises a spacer comprising a sequence selected from the group consisting of SEQ Identifiers: A528-A529 of Table 3A. In some embodiments, the PEgRNA comprises a spacer comprising a sequence as set forth in SEQ ID NOs. 876-877 of Table 3A. In some embodiments, the PEgRNA comprises a PBS comprising a sequence selected from the group consisting of SEQ Identifiers: B2043-B2051 of Table 3B. In some embodiments, the PEgRNA comprises a PBS comprising a sequence selected from the group consisting of SEQ ID NOs. 2802-2808 of Table 3B. In some embodiments, the PEgRNA comprises a PBS comprising a sequence selected from the group consisting of sequences AAATTCTT and AAATTCTTG as disclosed in Table 3B. In some embodiments, the PEgRNA comprises an editing template comprising a sequence selected from the group consisting of SEQ Identifiers: C10038-C10097 of Table 3C. In some embodiments, the PEgRNA comprises an editing template comprising a sequence selected from the group consisting of SEQ ID NOs: 14064-14123 of Table 3C. In some embodiments, the prime editing composition further comprises a ngRNA that comprises a spacer comprising a sequence selected from the group consisting of SEQ Identifiers: D916-D928, D1056, D1057, D1123, and D1124 of Table 3D. In some embodiments, the prime editing composition further comprises a ngRNA that comprises a spacer comprising a sequence selected from the group consisting of SEQ ID NOs. 20549-20561, 20689, 20690, 20756, and 20757 of Table 3D.

In some embodiments, the PEgRNA comprises a spacer comprising the of SEQ Identifier: A530 of Table 3A. In some embodiments, the PEgRNA comprises a spacer comprising a sequence as set forth in SEQ ID NO. 878 of Table 3A. In some embodiments, the PEgRNA comprises a PBS comprising a sequence selected from the group consisting of SEQ Identifiers: B2052-B2060 of Table 3B. In some embodiments, the PEgRNA comprises a PBS comprising a sequence selected from the group consisting of SEQ ID NOs. 2809-2815 of Table 3B. In some embodiments, the PEgRNA comprises a PBS comprising a sequence selected from the group consisting of sequences GAAATTCT and GAAATTCTT as disclosed in Table 3B. In some embodiments, the PEgRNA comprises an editing template comprising a sequence selected from the group consisting of SEQ Identifiers: C10098-C10128 of Table 3C. In some embodiments, the PEgRNA comprises an editing template comprising a sequence selected from the group consisting of SEQ ID NOs: 14124-14154 of Table 3C. In some embodiments, the prime editing composition further comprises a ngRNA that comprises a spacer comprising a sequence selected from the group consisting of SEQ Identifiers: D935-D944, and D1016, D1019, D1116-D1121 of Table 3D. In some embodiments, the prime editing composition further comprises a ngRNA that comprises a spacer comprising a sequence selected from the group consisting of SEQ ID NOs. 20568-20577, 20649, 20652, and 20749-20754, of Table 3D.

In some embodiments, the PEgRNA comprises a spacer comprising the sequence of SEQ Identifier: A531 of Table 3A. In some embodiments, the PEgRNA comprises a spacer comprising a sequence as set forth in SEQ ID NO. 879 of Table 3A. In some embodiments, the PEgRNA comprises a PBS comprising a sequence selected from the group consisting of SEQ Identifiers: B1836-B1844 of Table 3B. In some embodiments, the PEgRNA comprises a PBS comprising a sequence selected from the group consisting of SEQ ID NOs. 2641-2647 of Table 3B. In some embodiments, the PEgRNA comprises a PBS comprising a sequence selected from the group consisting of sequences TGAATAAC and TGAATAACT as disclosed in Table 3B. In some embodiments, the PEgRNA comprises an editing template comprising a sequence selected from the group consisting of SEQ Identifiers: C10129-C10159 of Table 3C. In some embodiments, the PEgRNA comprises an editing template comprising a sequence selected from the group consisting of SEQ ID NOs: 14155-14185 of Table 3C. In some embodiments, the prime editing composition further comprises a ngRNA that comprises a spacer comprising a sequence selected from the group consisting of SEQ Identifiers: D974-D981, D1002, D1033, D1034, D1035, D1036, D1037, D1055, D1073, D1104, D1105, D1129, and D1130 of Table 3D. In some embodiments, the prime editing composition further comprises a ngRNA that comprises a spacer comprising a sequence selected from the group consisting of SEQ ID NOs. 20607-20614, 20635, 20666, 20667, 20668, 20669, 20670, 20688, 20706, 20737, 20738, 20762, and 20763 of Table 3D.

In some embodiments, the PEgRNA comprises a spacer comprising a sequence selected from the group consisting of SEQ Identifiers: A511-A512 of Table 3A. In some embodiments, the PEgRNA comprises a spacer comprising a sequence as set forth in SEQ ID NO. 859 of Table 3A. In some embodiments, the PEgRNA comprises a PBS comprising a sequence selected from the group consisting of SEQ Identifiers: B1989-B1997 of Table 3B. In some embodiments, the PEgRNA comprises a PBS comprising a sequence selected from the group consisting of SEQ ID NOs. 2760-2766 of Table 3B. In some embodiments, the PEgRNA comprises a PBS comprising a sequence selected from the group consisting of sequences ACCTCCAC and ACCTCCACT as disclosed in Table 3B. In some embodiments, the PEgRNA comprises an editing template comprising a sequence selected from the group consisting of SEQ Identifiers: C10160-C10173 of Table 3C. In some embodiments, the PEgRNA comprises an editing template comprising a sequence selected from the group consisting of SEQ ID NOs: 14186-14199 of Table 3C. In some embodiments, the prime editing composition further comprises a ngRNA that comprises a spacer comprising a sequence selected from the group consisting of SEQ Identifiers: D933-D944, D947-D952, D1016, D1019, D1026, D1113, D1116, D1117, and D1118 of Table 3D. In some embodiments, the prime editing composition further comprises a ngRNA that comprises a spacer comprising a sequence selected from the group consisting of SEQ ID NOs. 20566-20577, 20580-20585, 20649, 20652, 20659, 20746, 20749, 20750, and 20751 of Table 3D.

In some embodiments, the PEgRNA comprises a spacer comprising the sequence of SEQ Identifier: A532 of Table 3A. In some embodiments, the PEgRNA comprises a spacer comprising a sequence as set forth in SEQ ID NO. 880 of Table 3A. In some embodiments, the PEgRNA comprises a PBS comprising a sequence selected from the group consisting of SEQ Identifiers: B2061-B2069 of Table 3B. In some embodiments, the PEgRNA comprises a PBS comprising a sequence selected from the group consisting of SEQ ID NOs. 2816-2822 of Table 3B. In some embodiments, the PEgRNA comprises a PBS comprising a sequence selected from the group consisting of sequences ATAACTAA and ATAACTAAT as disclosed in Table 3B. In some embodiments, the PEgRNA comprises an editing template comprising a sequence selected from the group consisting of SEQ Identifiers: C10174-C10204 of Table 3C. In some embodiments, the PEgRNA comprises an editing template comprising a sequence selected from the group consisting of SEQ ID NOs: 14200-14230 of Table 3C. In some embodiments, the prime editing composition further comprises a ngRNA that comprises a spacer comprising a sequence selected from the group consisting of SEQ Identifiers: D974-D981, D1002, D1033-D1037 D1055, D1073, D1104, D1105, D1129, and D1130 of Table 3D. In some embodiments, the prime editing composition further comprises a ngRNA that comprises a spacer comprising a sequence selected from the group consisting of SEQ ID NOs. 20607-20614, 20635, 20666-20670, 20688, 20706, 20737, 20738, 20762, and 20763 of Table 3D.

In some embodiments, the PEgRNA comprises a spacer comprising a sequence selected from the group consisting of SEQ Identifiers: A489-A490 of Table 3A. In some embodiments, the PEgRNA comprises a spacer comprising a sequence as set forth in SEQ ID NOs. 837-838 of Table 3A. In some embodiments, the PEgRNA comprises a PBS comprising a sequence selected from the group consisting of SEQ Identifiers: B1944-B1952 of Table 3B. In some embodiments, the PEgRNA comprises a PBS comprising a sequence selected from the group consisting of SEQ ID NOs. 2725-2731 of Table 3B. In some embodiments, the PEgRNA comprises a PBS comprising a sequence selected from the group consisting of sequences GTGATTCC and GTGATTCCA as disclosed in Table 3B. In some embodiments, the PEgRNA comprises an editing template comprising a sequence selected from the group consisting of SEQ Identifiers: C10205-C10206 of Table 3C. In some embodiments, the PEgRNA comprises an editing template comprising a sequence selected from the group consisting of SEQ ID NOs: 14231-14232 of Table 3C. In some embodiments, the prime editing composition further comprises a ngRNA that comprises a spacer comprising a sequence selected from the group consisting of SEQ Identifiers: D916-D928 D1056, and D1057 of Table 3D. In some embodiments, the prime editing composition further comprises a ngRNA that comprises a spacer comprising a sequence selected from the group consisting of SEQ ID NOs. 20549-20561, 20689, and 20690 of Table 3D.

In some embodiments, the PEgRNA comprises a spacer comprising the sequence of SEQ Identifier: A451 of Table 3A. In some embodiments, the PEgRNA comprises a spacer comprising a sequence as set forth in SEQ ID NO. 799 of Table 3A. In some embodiments, the PEgRNA comprises a PBS comprising a sequence selected from the group consisting of SEQ Identifiers: B1872-B1880 of Table 3B. In some embodiments, the PEgRNA comprises a PBS comprising a sequence selected from the group consisting of SEQ ID NOs. 2669-2675 of Table 3B. In some embodiments, the PEgRNA comprises a PBS comprising a sequence selected from the group consisting of sequences TGTGATTC and TGTGATTCC as disclosed in Table 3B. In some embodiments, the PEgRNA comprises an editing template comprising a sequence selected from the group consisting of SEQ Identifiers: C10207-C10208 of Table 3C. In some embodiments, the PEgRNA comprises an editing template comprising a sequence selected from the group consisting of SEQ ID NOs: 14233-14234 of Table 3C. In some embodiments, the prime editing composition further comprises a ngRNA that comprises a spacer comprising a sequence selected from the group consisting of SEQ Identifiers: 20566-20577, 20649, and 20652 of Table 3D. In some embodiments, the prime editing composition further comprises a ngRNA that comprises a spacer comprising a sequence selected from the group consisting of SEQ ID NOs. 20566-20577, 20649, and 20652 of Table 3D.

In some embodiments, the PEgRNA comprises a spacer comprising a sequence selected from the group consisting of SEQ Identifiers: A533-A534 of Table 3A. In some embodiments, the PEgRNA comprises a spacer comprising a sequence as set forth in SEQ ID NOs. 881-882 of Table 3A. In some embodiments, the PEgRNA comprises a PBS comprising a sequence selected from the group consisting of SEQ Identifiers: B2070-B2078 of Table 3B. In some embodiments, the PEgRNA comprises a PBS comprising a sequence selected from the group consisting of SEQ ID NOs. 2823-2829 of Table 3B. In some embodiments, the PEgRNA comprises a PBS comprising a sequence selected from the group consisting of sequences TCGTTGAC and TCGTTGACC as disclosed in Table 3B. In some embodiments, the PEgRNA comprises an editing template comprising a sequence selected from the group consisting of SEQ Identifiers: C10209-C10228 of Table 3C. In some embodiments, the PEgRNA comprises an editing template comprising a sequence selected from the group consisting of SEQ ID NOs: 14235-14254 of Table 3C. In some embodiments, the prime editing composition further comprises a ngRNA that comprises a spacer comprising a sequence selected from the group consisting of SEQ Identifiers: D934-D944, D948-D952, 20649, 20652, D1026, D1113, D1114, and 20749-D1119 of Table 3D. In some embodiments, the prime editing composition further comprises a ngRNA that comprises a spacer comprising a sequence selected from the group consisting of SEQ ID NOs. 20567-20577, 20581-20585, 20649, 20652, 20659, 20746, 20747, and 20749-20752 of Table 3D.

In some embodiments, the PEgRNA comprises a spacer comprising a sequence selected from the group consisting of SEQ Identifiers: A535-A537 of Table 3A. In some embodiments, the PEgRNA comprises a spacer comprising a sequence as set forth in SEQ ID NOs. 883-885 of Table 3A. In some embodiments, the PEgRNA comprises a PBS comprising a sequence selected from the group consisting of SEQ Identifiers: B2079-B2087 of Table 3B. In some embodiments, the PEgRNA comprises a PBS comprising a sequence selected from the group consisting of SEQ ID NOs. 2830-2836 of Table 3B. In some embodiments, the PEgRNA comprises a PBS comprising a sequence selected from the group consisting of sequences GCTAAAGA and GCTAAAGAA as disclosed in Table 3B. In some embodiments, the PEgRNA comprises an editing template comprising a sequence selected from the group consisting of SEQ Identifiers: C10229-C10259 of Table 3C. In some embodiments, the PEgRNA comprises an editing template comprising a sequence selected from the group consisting of SEQ ID NOs: 14255-14285 of Table 3C. In some embodiments, the prime editing composition further comprises a ngRNA that comprises a spacer comprising a sequence selected from the group consisting of SEQ Identifiers: D925, D1030, and D1032 of Table 3D. In some embodiments, the prime editing composition further comprises a ngRNA that comprises a spacer comprising a sequence selected from the group consisting of SEQ ID NOs. 20558, 20663, and 20665 of Table 3D.

In some embodiments, the PEgRNA comprises a spacer comprising a sequence selected from the group consisting of SEQ Identifiers: A506, A507, A513, and A514 of Table 3A. In some embodiments, the PEgRNA comprises a spacer comprising a sequence as set forth in SEQ ID NOs. 854, 855, 861, and 862 of Table 3A. In some embodiments, the PEgRNA comprises a PBS comprising a sequence selected from the group consisting of SEQ Identifiers: B1926-B1934 of Table 3B. In some embodiments, the PEgRNA comprises a PBS comprising a sequence selected from the group consisting of SEQ ID NOs. 2711-2717 of Table 3B. In some embodiments, the PEgRNA comprises a PBS comprising a sequence selected from the group consisting of sequences TCAGTGTG and TCAGTGTGA as disclosed in Table 3B. In some embodiments, the PEgRNA comprises an editing template comprising a sequence selected from the group consisting of SEQ Identifiers: C10260-C10271 of Table 3C. In some embodiments, the PEgRNA comprises an editing template comprising a sequence selected from the group consisting of SEQ ID NOs: 14286-14297 of Table 3C. In some embodiments, the prime editing composition further comprises a ngRNA that comprises a spacer comprising a sequence selected from the group consisting of SEQ Identifiers: D916-D928, D1021, D1023, D1025, D1056, and D1057 of Table 3D. In some embodiments, the prime editing composition further comprises a ngRNA that comprises a spacer comprising a sequence selected from the group consisting of SEQ ID NOs. 20549-20561, 20654, 20656, 20658, 20689, 20690 of Table 3D.

In some embodiments, the PEgRNA comprises a spacer comprising a sequence selected from the group consisting of SEQ Identifiers: A503-A504 of Table 3A. In some embodiments, the PEgRNA comprises a spacer comprising a sequence as set forth in SEQ ID NOs. 851-852 of Table 3A. In some embodiments, the PEgRNA comprises a PBS comprising a sequence selected from the group consisting of SEQ Identifiers: B1953-B1961 of Table 3B. In some embodiments, the PEgRNA comprises a PBS comprising a sequence selected from the group consisting of SEQ ID NOs. 2732-2738 of Table 3B. In some embodiments, the PEgRNA comprises a PBS comprising a sequence selected from the group consisting of sequences CTCAGTGT and CTCAGTGTG as disclosed in Table 3B. In some embodiments, the PEgRNA comprises an editing template comprising a sequence selected from the group consisting of SEQ Identifiers: C10272-C10278 of Table 3C. In some embodiments, the PEgRNA comprises an editing template comprising a sequence selected from the group consisting of SEQ ID NOs: 14298-14304 of Table 3C. In some embodiments, the prime editing composition further comprises a ngRNA that comprises a spacer comprising a sequence selected from the group consisting of SEQ Identifiers: D933-D944, D1016, D1018, D1019, D1116, and D1117 of Table 3D. In some embodiments, the prime editing composition further comprises a ngRNA that comprises a spacer comprising a sequence selected from the group consisting of SEQ ID NOs. 20566-20577, 20649, 20651, 20652, 20749, 20750 of Table 3D.

In some embodiments, the PEgRNA comprises a spacer comprising the sequence of SEQ Identifier: A505 of Table 3A. In some embodiments, the PEgRNA comprises a spacer comprising a sequence as set forth in SEQ ID NO. 853 of Table 3A. In some embodiments, the PEgRNA comprises a PBS comprising a sequence selected from the group consisting of SEQ Identifiers: B1962-B1970 of Table 3B. In some embodiments, the PEgRNA comprises a PBS comprising a sequence selected from the group consisting of SEQ ID NOs. 2739-2745 of Table 3B. In some embodiments, the PEgRNA comprises a PBS comprising a sequence selected from the group consisting of sequences ACTCAGTG and ACTCAGTGT as disclosed in Table 3B. In some embodiments, the PEgRNA comprises an editing template comprising a sequence selected from the group consisting of SEQ Identifiers: C10279-C10286 of Table 3C. In some embodiments, the PEgRNA comprises an editing template comprising a sequence selected from the group consisting of SEQ ID NOs: 14305-14312 of Table 3C. In some embodiments, the prime editing composition further comprises a ngRNA that comprises a spacer comprising a sequence selected from the group consisting of SEQ Identifiers: D933-D944, D1016, D1019, D1116, D1117, and D1118 of Table 3D. In some embodiments, the prime editing composition further comprises a ngRNA that comprises a spacer comprising a sequence selected from the group consisting of SEQ ID NOs. 20566-20577, 20649, 20652, 20749, 20750, and 20751 of Table 3D.

In some embodiments, the PEgRNA comprises a spacer comprising the sequence of SEQ Identifier: A538 of Table 3A. In some embodiments, the PEgRNA comprises a spacer comprising a sequence as set forth in SEQ ID NO. 886 of Table 3A. In some embodiments, the PEgRNA comprises a PBS comprising a sequence selected from the group consisting of SEQ Identifiers: B2088-B2096 of Table 3B. In some embodiments, the PEgRNA comprises a PBS comprising a sequence selected from the group consisting of SEQ ID NOs. 2837-2843 of Table 3B. In some embodiments, the PEgRNA comprises a PBS comprising a sequence selected from the group consisting of sequences GCTGTAAA and GCTGTAAAT as disclosed in Table 3B. In some embodiments, the PEgRNA comprises an editing template comprising a sequence selected from the group consisting of SEQ Identifiers: C10287-C10292 of Table 3C. In some embodiments, the PEgRNA comprises an editing template comprising a sequence selected from the group consisting of SEQ ID NOs:14313-14318 of Table 3C. In some embodiments, the prime editing composition further comprises a ngRNA that comprises a spacer comprising a sequence selected from the group consisting of SEQ Identifiers: D978-D981, D1002, D1033-D1037, D1055, D1073, D1104, D1105, and D1131-D1135 of Table 3D. In some embodiments, the prime editing composition further comprises a ngRNA that comprises a spacer comprising a sequence selected from the group consisting of SEQ ID NOs. 20611-20614, 20635, 20666-20670, 20688, 20706, 20737, 20738, 20764-20768 of Table 3D.

In some embodiments, the PEgRNA comprises a spacer comprising the sequence of SEQ Identifier: A539 of Table 3A. In some embodiments, the PEgRNA comprises a spacer comprising a sequence as set forth in SEQ ID NO. 887 of Table 3A. In some embodiments, the PEgRNA comprises a PBS comprising a sequence selected from the group consisting of SEQ Identifiers: B2097-B2105 of Table 3B. In some embodiments, the PEgRNA comprises a PBS comprising a sequence selected from the group consisting of SEQ ID NOs. 2844-2850 of Table 3B. In some embodiments, the PEgRNA comprises a PBS comprising a sequence selected from the group consisting of sequences ATTTGCTG and ATTTGCTGT as disclosed in Table 3B. In some embodiments, the PEgRNA comprises an editing template comprising a sequence selected from the group consisting of SEQ Identifiers: C10293-C10302 of Table 3C. In some embodiments, the PEgRNA comprises an editing template comprising a sequence selected from the group consisting of SEQ ID NOs: 14319-14328 of Table 3C. In some embodiments, the prime editing composition further comprises a ngRNA that comprises a spacer comprising a sequence selected from the group consisting of SEQ Identifiers: D977-D981, D1002, D1033-D1037, D1055, D1073, D1104, 1105, and D1131-D1135 of Table 3D. In some embodiments, the prime editing composition further comprises a ngRNA that comprises a spacer comprising a sequence selected from the group consisting of SEQ ID NOs. 20610-20614, 20635, 20666-20670, 20688, 20706, 20737, 20738, and 20764-20768 of Table 3D.

In some embodiments, the PEgRNA comprises a spacer comprising a sequence selected from the group consisting of SEQ Identifiers: A540-A543 of Table 3A. In some embodiments, the PEgRNA comprises a spacer comprising a sequence as set forth in SEQ ID NOs. 888-891 of Table 3A. In some embodiments, the PEgRNA comprises a PBS comprising a sequence selected from the group consisting of SEQ Identifiers: B2106-B2114 of Table 3B. In some embodiments, the PEgRNA comprises a PBS comprising a sequence selected from the group consisting of SEQ ID NOs. 2851-2857 of Table 3B. In some embodiments, the PEgRNA comprises a PBS comprising a sequence selected from the group consisting of sequences CGTTGACC and CGTTGACCT as disclosed in Table 3B. In some embodiments, the PEgRNA comprises an editing template comprising a sequence selected from the group consisting of SEQ Identifiers: C10303-C10340 of Table 3C. In some embodiments, the PEgRNA comprises an editing template comprising a sequence selected from the group consisting of SEQ ID NOs:14329-14366 of Table 3C. In some embodiments, the prime editing composition further comprises a ngRNA that comprises a spacer comprising a sequence selected from the group consisting of SEQ Identifiers: D916-D928, D1028-D1032, D1056, D1057, D1123, D1124, D1136, and D1137 of Table 3D. In some embodiments, the prime editing composition further comprises a ngRNA that comprises a spacer comprising a sequence selected from the group consisting of SEQ ID NOs. 20549-20561, 20661-20665, 20689, 20690, 20756, 20757, 20769, and 20770 of Table 3D.

In some embodiments, the PEgRNA comprises a spacer comprising a sequence selected from the group consisting of SEQ Identifiers: A517-A518 of Table 3A. In some embodiments, the PEgRNA comprises a spacer comprising a sequence as set forth in SEQ ID NOs. 865-866 of Table 3A. In some embodiments, the PEgRNA comprises a PBS comprising a sequence selected from the group consisting of SEQ Identifiers: B1998-B2006 of Table 3B. In some embodiments, the PEgRNA comprises a PBS comprising a sequence selected from the group consisting of SEQ ID NOs. 2767-2773 of Table 3B. In some embodiments, the PEgRNA comprises a PBS comprising a sequence selected from the group consisting of sequences ATTGGTCT and ATTGGTCTA as disclosed in Table 3B. In some embodiments, the PEgRNA comprises an editing template comprising a sequence selected from the group consisting of SEQ Identifiers: C10341-C10390 of Table 3C. In some embodiments, the PEgRNA comprises an editing template comprising a sequence selected from the group consisting of SEQ ID NOs: 14367-14416 of Table 3C. In some embodiments, the prime editing composition further comprises a ngRNA that comprises a spacer comprising a sequence selected from the group consisting of SEQ Identifiers: D990-D992, D999-D1001, D1109-D1112, and D1125-D1128 of Table 3D. In some embodiments, the prime editing composition further comprises a ngRNA that comprises a spacer comprising a sequence selected from the group consisting of SEQ ID NOs. 20623-20625, 20632-20634, 20742-20745, and 20758-20761 of Table 3D.

In some embodiments, the PEgRNA comprises a spacer comprising the sequence of SEQ Identifier: A519 of Table 3A. In some embodiments, the PEgRNA comprises a spacer comprising a sequence as set forth in SEQ ID NO.867 of Table 3A. In some embodiments, the PEgRNA comprises a PBS comprising a sequence selected from the group consisting of SEQ Identifiers: B2007-B2015 of Table 3B. In some embodiments, the PEgRNA comprises a PBS comprising a sequence selected from the group consisting of SEQ ID NOs. 2774-2780 of Table 3B. In some embodiments, the PEgRNA comprises a PBS comprising a sequence selected from the group consisting of sequences TATTGGTC and TATTGGTCT as disclosed in Table 3B. In some embodiments, the PEgRNA comprises an editing template comprising a sequence selected from the group consisting of SEQ Identifiers: C10391-C10416 of Table 3C. In some embodiments, the PEgRNA comprises an editing template comprising a sequence selected from the group consisting of SEQ ID NOs:14417-14442 of Table 3C. In some embodiments, the prime editing composition further comprises a ngRNA that comprises a spacer comprising a sequence selected from the group consisting of SEQ Identifiers: D974-D981, D1002, D1033-D1037, D1055, D1073, D1104, D1105, D1130-D1134 of Table 3D. In some embodiments, the prime editing composition further comprises a ngRNA that comprises a spacer comprising a sequence selected from the group consisting of SEQ ID NOs. 20607-20614, 20635, 20666-20670, 20688, 20706, 20737, 20738, and 20763-20767 of Table 3D.

In some embodiments, the PEgRNA comprises a spacer comprising a sequence selected from the group consisting of SEQ Identifiers: A544-A545 of Table 3A. In some embodiments, the PEgRNA comprises a spacer comprising a sequence as set forth in SEQ ID NOs. 892-893 of Table 3A. In some embodiments, the PEgRNA comprises a PBS comprising a sequence selected from the group consisting of SEQ Identifiers: B2115-B2123 of Table 3B. In some embodiments, the PEgRNA comprises a PBS comprising a sequence selected from the group consisting of SEQ ID NOs. 2858-2864 of Table 3B. In some embodiments, the PEgRNA comprises a PBS comprising a sequence selected from the group consisting of sequences AAGCATTT and AAGCATTTG as disclosed in Table 3B. In some embodiments, the PEgRNA comprises an editing template comprising a sequence selected from the group consisting of SEQ Identifiers: C10417-C10430 of Table 3C. In some embodiments, the PEgRNA comprises an editing template comprising a sequence selected from the group consisting of SEQ ID NOs:14443-14456 of Table 3C. In some embodiments, the prime editing composition further comprises a ngRNA that comprises a spacer comprising a sequence selected from the group consisting of SEQ Identifiers: D960, D976-D981, D1002, D1033-D1037, D1040, D1041, D1055, D1073, D1104, D1105, D1131-D1135 and D1138 of Table 3D. In some embodiments, the prime editing composition further comprises a ngRNA that comprises a spacer comprising a sequence selected from the group consisting of SEQ ID NOs. 20593, 20609-20614, 20635, 20666-20670, 20673, 20674, 20688, 20706, 20737, 20738, 20764-20768, and 20771 of Table 3D.

In some embodiments, the editing target sequence is in exon 14 of the CFTR gene.

In some embodiments, the editing target sequence or the complementary sequence on the CFTR gene thereof comprises a mutation that encodes an amino acid substitution S684Y and/or E681V relative to a wild type CFTR polypeptide set forth in SEQ ID NO: 19, and wherein the editing template encodes an intended nucleotide edit that corrects the mutation. In some embodiments, the editing target sequence or the complementary sequence on the CFTR gene thereof comprises a deletion of nucleotides AA and insertion of nucleotide G (c.2051_2052delAAinsG) at position 117,592,218, a A→T mutation (c.2042A>T) at position 117,592,209, a deletion of A (c.2044delA) at position 117,592,211, a duplication of a C (c.2045dupC) at position 117,592,212, a deletion of AA (c.2051_2052delAA) at position 117,592,218, a duplication of a nucleotide A (c.2052dupA) at position 117,592,219, a deletion of A (c.2052delA) at position 117,592,219, a duplication of a C (c.2053dupC) at position 117,592,220, a C→A mutation (c.2057C>A) at position 117,592,224, a duplication of nucleotides TTTT (c.2058_2061dupTTTT) at position 117,592,228, and/or a deletion of nucleotide T (c.2061delT) at position 117,592,228 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide. In some embodiments, the editing template comprises a wild type CFTR gene sequence. In some embodiments, the editing template comprises one or more intended nucleotide edits compared to the target CFTR gene sequence in exon 14 of the target CFTR gene. Accordingly, in some embodiments, the newly synthesized single stranded DNA comprises one or more intended nucleotide edits compared to the target CFTR gene sequence. In some embodiments, the newly synthesized single stranded DNA encoded by the editing template is capable of correcting the c.2051_2052delAAinsG mutation, the c.2042A>T mutation, the c.2044delA mutation, the c.2045dupC mutation, the c.2051_2052delAA mutation, the c.2052dupA mutation, the c.2052delA mutation, the c.2053dupC mutation, the c.2057C>A mutation, the c.2058_2061dupTTTT mutation, and/or the c.2061delT mutation in the target CFTR gene. In some embodiments, the newly synthesized single stranded DNA encoded by the editing template is capable of correcting all of the c.2051_2052delAAinsG mutation, the c.2042A>T mutation, the c.2044delA mutation, the c.2045dupC mutation, the c.2051_2052delAA mutation, the c.2052dupA mutation, the c.2052delA mutation, the c.2053dupC mutation, the c.2057C>A mutation, the c.2058_2061dupTTTT mutation, and the c.2061delT mutation in the target CFTR gene. In some embodiments, two or more of the c.2051_2052delAAinsG mutation, the c.2042A>T mutation, the c.2044delA mutation, the c.2045dupC mutation, the c.2051_2052delAA mutation, the c.2052dupA mutation, the c.2052delA mutation, the c.2053dupC mutation, the c.2057C>A mutation, the c.2058_2061dupTTTT mutation, and the c.2061delT mutation are located on the same CFTR gene. In some embodiments, the newly synthesized single stranded DNA encoded by the editing template is capable of correcting the c.2051_2052delAAinsG mutation, the c.2042A>T mutation, the c.2044delA mutation, the c.2045dupC mutation, the c.2051_2052delAA mutation, the c.2052dupA mutation, the c.2052delA mutation, the c.2053dupC mutation, the c.2057C>A mutation, the c.2058_2061dupTTTT mutation, and the c.2061delT mutation that are located on different CFTR genes. For example, in some embodiments, a subject may comprise two copies of CFTR genes, each comprising a different mutation selected from the group consisting of the c.2051_2052delAAinsG mutation, the c.2042A>T mutation, the c.2044delA mutation, the c.2045dupC mutation, the c.2051_2052delAA mutation, the c.2052dupA mutation, the c.2052delA mutation, the c.2053dupC mutation, the c.2057C>A mutation, the c.2058_2061dupTTTT mutation, and the c.2061delT mutation, all of which may be corrected by an editing template of a single PEgRNA. In some embodiments, the editing template is capable of correcting one or more, or all of the c.2051_2052delAAinsG mutation, the c.2042A>T mutation, the c.2044delA mutation, the c.2045dupC mutation, the c.2051_2052delAA mutation, the c.2052dupA mutation, the c.2052delA mutation, the c.2053dupC mutation, the c.2057C>A mutation, the c.2058_2061dupTTTT mutation, and the c.2061delT mutation that are in different subjects, wherein each subject may have one or more of the mutations.

In some embodiments, the PEgRNA comprises a spacer comprising a sequence selected from the group consisting of SEQ Identifiers: A546-A547 of Table 3A. In some embodiments, the PEgRNA comprises a spacer comprising a sequence as set forth in SEQ ID NOs. 894-895 of Table 3A. In some embodiments, the PEgRNA comprises a PBS comprising a sequence selected from the group consisting of SEQ Identifiers: B2124-B2132 of Table 3B. In some embodiments, the PEgRNA comprises a PBS comprising a sequence selected from the group consisting of SEQ ID NOs. 2865-2871 of Table 3B. In some embodiments, the PEgRNA comprises a PBS comprising a sequence selected from the group consisting of sequences AACAGACT and AACAGACTG as disclosed in Table 3B. In some embodiments, the PEgRNA comprises an editing template comprising a sequence selected from the group consisting of SEQ Identifiers: C10431-C10486 of Table 3C. In some embodiments, the PEgRNA comprises an editing template comprising a sequence selected from the group consisting of SEQ ID NOs:14457-14512 of Table 3C. In some embodiments, the prime editing composition further comprises a ngRNA that comprises a spacer comprising a sequence selected from the group consisting of SEQ Identifiers: D1139-D1156 of Table 3D. In some embodiments, the prime editing composition further comprises a ngRNA that comprises a spacer comprising a sequence selected from the group consisting of SEQ ID NOs. 20772-20789 of Table 3D.

In some embodiments, the PEgRNA comprises a spacer comprising a sequence selected from the group consisting of SEQ Identifiers: A548-A549 of Table 3A. In some embodiments, the PEgRNA comprises a spacer comprising a sequence as set forth in SEQ ID NOs. 896-897 of Table 3A. In some embodiments, the PEgRNA comprises a PBS comprising a sequence selected from the group consisting of SEQ Identifiers: B2133-B2141 of Table 3B. In some embodiments, the PEgRNA comprises a PBS comprising a sequence selected from the group consisting of SEQ ID NOs. 2872-2878 of Table 3B. In some embodiments, the PEgRNA comprises a PBS comprising a sequence selected from the group consisting of sequences TAATGAGA and TAATGAGAA as disclosed in Table 3B. In some embodiments, the PEgRNA comprises an editing template comprising the sequence of SEQ Identifier: C10487 of Table 3C. In some embodiments, the PEgRNA comprises an editing template comprising a sequence selected from the group consisting of SEQ ID NOs:14513 of Table 3C. In some embodiments, the prime editing composition further comprises a ngRNA that comprises a spacer comprising a sequence selected from the group consisting of SEQ Identifiers: D1157-D1188 of Table 3D. In some embodiments, the prime editing composition further comprises a ngRNA that comprises a spacer comprising a sequence selected from the group consisting of SEQ ID NOs. 20790-20821 of Table 3D.

In some embodiments, the PEgRNA comprises a spacer comprising a sequence selected from the group consisting of SEQ Identifiers: A550-A551 of Table 3A. In some embodiments, the PEgRNA comprises a spacer comprising a sequence as set forth in SEQ ID NOs. 898-899 of Table 3A. In some embodiments, the PEgRNA comprises a PBS comprising a sequence selected from the group consisting of SEQ Identifiers: B2142-B2150 of Table 3B. In some embodiments, the PEgRNA comprises a PBS comprising a sequence selected from the group consisting of SEQ ID NOs. 2879-2885 of Table 3B. In some embodiments, the PEgRNA comprises a PBS comprising a sequence selected from the group consisting of sequences TCTAATGA and TCTAATGAG as disclosed in Table 3B. In some embodiments, the PEgRNA comprises an editing template comprising a sequence selected from the group consisting of SEQ Identifiers: C10488-C10490 of Table 3C. In some embodiments, the PEgRNA comprises an editing template comprising a sequence selected from the group consisting of SEQ ID NOs:14514-14516 of Table 3C. In some embodiments, the prime editing composition further comprises a ngRNA that comprises a spacer comprising a sequence selected from the group consisting of SEQ Identifiers: D1157-D1190 of Table 3D. In some embodiments, the prime editing composition further comprises a ngRNA that comprises a spacer comprising a sequence selected from the group consisting of SEQ ID NOs. 20790-20823 of Table 3D.

In some embodiments, the PEgRNA comprises a spacer comprising a sequence selected from the group consisting of SEQ Identifiers: A552-A553 of Table 3A. In some embodiments, the PEgRNA comprises a spacer comprising a sequence as set forth in SEQ ID NOs. 900-901 of Table 3A. In some embodiments, the PEgRNA comprises a PBS comprising a sequence selected from the group consisting of SEQ Identifiers: B2151-B2159 of Table 3B. In some embodiments, the PEgRNA comprises a PBS comprising a sequence selected from the group consisting of SEQ ID NOs. 2886-2892 of Table 3B. In some embodiments, the PEgRNA comprises a PBS comprising a sequence selected from the group consisting of sequences AAACAGAC and AAACAGACT as disclosed in Table 3B. In some embodiments, the PEgRNA comprises an editing template comprising a sequence selected from the group consisting of SEQ Identifiers: C10491-C10519 of Table 3C. In some embodiments, the PEgRNA comprises an editing template comprising a sequence selected from the group consisting of SEQ ID NOs:14517-14545 of Table 3C. In some embodiments, the prime editing composition further comprises a ngRNA that comprises a spacer comprising a sequence selected from the group consisting of SEQ Identifiers: D1191-D1227 of Table 3D. In some embodiments, the prime editing composition further comprises a ngRNA that comprises a spacer comprising a sequence selected from the group consisting of SEQ ID NOs. 20824-20860 of Table 3D.

In some embodiments, the PEgRNA comprises a spacer comprising a sequence selected from the group consisting of SEQ Identifiers: A554-A555 of Table 3A. In some embodiments, the PEgRNA comprises a spacer comprising a sequence as set forth in SEQ ID NOs. 902-903 of Table 3A. In some embodiments, the PEgRNA comprises a PBS comprising a sequence selected from the group consisting of SEQ Identifiers: B2160-B2168 of Table 3B. In some embodiments, the PEgRNA comprises a PBS comprising a sequence selected from the group consisting of SEQ ID NOs. 2893-2899 of Table 3B. In some embodiments, the PEgRNA comprises a PBS comprising a sequence selected from the group consisting of sequences GACAGGAG and GACAGGAGC as disclosed in Table 3B. In some embodiments, the PEgRNA comprises an editing template comprising a sequence selected from the group consisting of SEQ Identifiers: C10520-C10538 of Table 3C. In some embodiments, the PEgRNA comprises an editing template comprising a sequence selected from the group consisting of SEQ ID NOs:14546-14564 of Table 3C. In some embodiments, the prime editing composition further comprises a ngRNA that comprises a spacer comprising a sequence selected from the group consisting of SEQ Identifiers: D1166-D1190, and D1228-D1234 of Table 3D. In some embodiments, the prime editing composition further comprises a ngRNA that comprises a spacer comprising a sequence selected from the group consisting of SEQ ID NOs. 20799-20823, and 20861-20867 of Table 3D.

In some embodiments, the PEgRNA comprises a spacer comprising a sequence selected from the group consisting of SEQ Identifiers: A556-A557 of Table 3A. In some embodiments, the PEgRNA comprises a spacer comprising a sequence as set forth in SEQ ID NOs. 904-905 of Table 3A. In some embodiments, the PEgRNA comprises a PBS comprising a sequence selected from the group consisting of SEQ Identifiers: B2169-B2177 of Table 3B. In some embodiments, the PEgRNA comprises a PBS comprising a sequence selected from the group consisting of SEQ ID NOs. 2900-2906 of Table 3B. In some embodiments, the PEgRNA comprises a PBS comprising a sequence selected from the group consisting of sequences CAGGAGAC and CAGGAGACA as disclosed in Table 3B. In some embodiments, the PEgRNA comprises an editing template comprising a sequence selected from the group consisting of SEQ Identifiers: C10539-C10562 of Table 3C. In some embodiments, the PEgRNA comprises an editing template comprising a sequence selected from the group consisting of SEQ ID NOs:14565-14588 of Table 3C. In some embodiments, the prime editing composition further comprises a ngRNA that comprises a spacer comprising a sequence selected from the group consisting of SEQ Identifiers: D1158-D1190, D1228, D1229, D1235, D1236, D1237, and D1238 of Table 3D. In some embodiments, the prime editing composition further comprises a ngRNA that comprises a spacer comprising a sequence selected from the group consisting of SEQ ID NOs. 20791-20823, 20861, 20862, 20868, 20869, 20870, and 20871 of Table 3D.

In some embodiments, the PEgRNA comprises a spacer comprising a sequence selected from the group consisting of SEQ Identifiers: A558-A559 of Table 3A. In some embodiments, the PEgRNA comprises a spacer comprising a sequence as set forth in SEQ ID NOs. 906-907 of Table 3A. In some embodiments, the PEgRNA comprises a PBS comprising a sequence selected from the group consisting of SEQ Identifiers: B2178-B2186 of Table 3B. In some embodiments, the PEgRNA comprises a PBS comprising a sequence selected from the group consisting of SEQ ID NOs. 2907-2913 of Table 3B. In some embodiments, the PEgRNA comprises a PBS comprising a sequence selected from the group consisting of sequences CTAATGAG and CTAATGAGA as disclosed in Table 3B. In some embodiments, the PEgRNA comprises an editing template comprising a sequence selected from the group consisting of SEQ Identifiers: C10563-C10566 of Table 3C. In some embodiments, the PEgRNA comprises an editing template comprising a sequence selected from the group consisting of SEQ ID NOs:14589-14592 of Table 3C. In some embodiments, the prime editing composition further comprises a ngRNA that comprises a spacer comprising a sequence selected from the group consisting of SEQ Identifiers: D1239-D1259 of Table 3D. In some embodiments, the prime editing composition further comprises a ngRNA that comprises a spacer comprising a sequence selected from the group consisting of SEQ ID NOs. 20872-20892 of Table 3D.

In some embodiments, the PEgRNA comprises a spacer comprising the sequence of SEQ Identifier: A560 of Table 3A. In some embodiments, the PEgRNA comprises a spacer comprising a sequence as set forth in SEQ ID NO. 908 of Table 3A In some embodiments, the PEgRNA comprises a PBS comprising a sequence selected from the group consisting of SEQ Identifiers: B2187-B2195 of Table 3B. In some embodiments, the PEgRNA comprises a PBS comprising a sequence selected from the group consisting of SEQ ID NOs. 2914-2920 of Table 3B. In some embodiments, the PEgRNA comprises a PBS comprising a sequence selected from the group consisting of sequences CCTTCTAA and CCTTCTAAT as disclosed in Table 3B. In some embodiments, the PEgRNA comprises an editing template comprising a sequence selected from the group consisting of SEQ Identifiers: C10567-C10572 of Table 3C. In some embodiments, the PEgRNA comprises an editing template comprising a sequence selected from the group consisting of SEQ ID NOs:14593-14598 of Table 3C. In some embodiments, the prime editing composition further comprises a ngRNA that comprises a spacer comprising a sequence selected from the group consisting of SEQ Identifiers: D1165-D1188, and D1190 of Table 3D. In some embodiments, the prime editing composition further comprises a ngRNA that comprises a spacer comprising a sequence selected from the group consisting of SEQ ID NOs. 20798-20821, and 20823 of Table 3D.

In some embodiments, the PEgRNA comprises a spacer comprising the sequence of SEQ Identifier: A561 of Table 3A. In some embodiments, the PEgRNA comprises a spacer comprising a sequence as set forth in SEQ ID NO. 909 of Table 3A. In some embodiments, the PEgRNA comprises a PBS comprising a sequence selected from the group consisting of SEQ Identifiers: B2196-B2204 of Table 3B. In some embodiments, the PEgRNA comprises a PBS comprising a sequence selected from the group consisting of SEQ ID NOs. 2921-2927 of Table 3B. In some embodiments, the PEgRNA comprises a PBS comprising a sequence selected from the group consisting of sequences TTTTAAAC and TTTTAAACA as disclosed in Table 3B. In some embodiments, the PEgRNA comprises an editing template comprising a sequence selected from the group consisting of SEQ Identifiers: C10573-C10603 of Table 3C. In some embodiments, the PEgRNA comprises an editing template comprising a sequence selected from the group consisting of SEQ ID NOs:14599-14629 of Table 3C. In some embodiments, the prime editing composition further comprises a ngRNA that comprises a spacer comprising a sequence selected from the group consisting of SEQ Identifiers: D1191-D1213 of Table 3D. In some embodiments, the prime editing composition further comprises a ngRNA that comprises a spacer comprising a sequence selected from the group consisting of SEQ ID NOs. 20824-20846 of Table 3D.

In some embodiments, the PEgRNA comprises a spacer comprising a sequence selected from the group consisting of SEQ Identifiers: A562-A563 of Table 3A. In some embodiments, the PEgRNA comprises a spacer comprising a sequence as set forth in SEQ ID NOs. 910-911 of Table 3A. In some embodiments, the PEgRNA comprises a PBS comprising a sequence selected from the group consisting of SEQ Identifiers: B2205-B2213 of Table 3B. In some embodiments, the PEgRNA comprises a PBS comprising a sequence selected from the group consisting of SEQ ID NOs. 2928-2934 of Table 3B. In some embodiments, the PEgRNA comprises a PBS comprising a sequence selected from the group consisting of sequences AGTTTGGG and AGTTTGGGG as disclosed in Table 3B. In some embodiments, the PEgRNA comprises an editing template comprising a sequence selected from the group consisting of SEQ Identifiers: C10604-C10635 of Table 3C. In some embodiments, the PEgRNA comprises an editing template comprising a sequence selected from the group consisting of SEQ ID NOs:14630-14661 of Table 3C. In some embodiments, the prime editing composition further comprises a ngRNA that comprises a spacer comprising a sequence selected from the group consisting of SEQ Identifiers: D1141-D1145, D1150-D1154, and D1260-D1267 of Table 3D. In some embodiments, the prime editing composition further comprises a ngRNA that comprises a spacer comprising a sequence selected from the group consisting of SEQ ID NOs. 20774-20778, 20783-20788, and 20893, 20900 of Table 3D.

In some embodiments, the PEgRNA comprises a spacer comprising the sequence of SEQ Identifier: A564 of Table 3A. In some embodiments, the PEgRNA comprises a spacer comprising a sequence as set forth in SEQ ID NO. 912 of Table 3A. In some embodiments, the PEgRNA comprises a PBS comprising a sequence selected from the group consisting of SEQ Identifiers: B2214-B2222 of Table 3B. In some embodiments, the PEgRNA comprises a PBS comprising a sequence selected from the group consisting of SEQ ID NOs. 2935-2941 of Table 3B. In some embodiments, the PEgRNA comprises a PBS comprising a sequence selected from the group consisting of sequences GAGTTTGG and GAGTTTGGG as disclosed in Table 3B. In some embodiments, the PEgRNA comprises an editing template comprising a sequence selected from the group consisting of SEQ Identifiers: C10636-C10652 of Table 3C. In some embodiments, the PEgRNA comprises an editing template comprising a sequence selected from the group consisting of SEQ ID NOs:14662-14678 of Table 3C. In some embodiments, the prime editing composition further comprises a ngRNA that comprises a spacer comprising a sequence selected from the group consisting of SEQ Identifiers: D1196-D1214, and D1268-D1270 of Table 3D. In some embodiments, the prime editing composition further comprises a ngRNA that comprises a spacer comprising a sequence selected from the group consisting of SEQ ID NOs. 20829-20847, and 20901-20903 of Table 3D.

In some embodiments, the PEgRNA comprises a spacer comprising a sequence selected from the group consisting of SEQ Identifiers: A565-A567 of Table 3A. In some embodiments, the PEgRNA comprises a spacer comprising a sequence as set forth in SEQ ID NOs. 913-915 of Table 3A. In some embodiments, the PEgRNA comprises a PBS comprising a sequence selected from the group consisting of SEQ Identifiers: B2223-B2231 of Table 3B. In some embodiments, the PEgRNA comprises a PBS comprising a sequence selected from the group consisting of SEQ ID NOs. 2942-2948 of Table 3B. In some embodiments, the PEgRNA comprises a PBS comprising a sequence selected from the group consisting of sequences ACAGGAGC and ACAGGAGCA as disclosed in Table 3B. In some embodiments, the PEgRNA comprises an editing template comprising a sequence selected from the group consisting of SEQ Identifiers: C10653-C10670 of Table 3C. In some embodiments, the PEgRNA comprises an editing template comprising a sequence selected from the group consisting of SEQ ID NOs:14679-14696 of Table 3C. In some embodiments, the prime editing composition further comprises a ngRNA that comprises a spacer comprising a sequence selected from the group consisting of SEQ Identifiers: D1244, D1248, and D1271-D1283 of Table 3D. In some embodiments, the prime editing composition further comprises a ngRNA that comprises a spacer comprising a sequence selected from the group consisting of SEQ ID NOs. 20877-20881, and 20904-20916 of Table 3D.

In some embodiments, the PEgRNA comprises a spacer comprising a sequence selected from the group consisting of SEQ Identifiers: A568-A569 of Table 3A. In some embodiments, the PEgRNA comprises a spacer comprising a sequence as set forth in SEQ ID NOs. 916-917 of Table 3A. In some embodiments, the PEgRNA comprises a PBS comprising a sequence selected from the group consisting of SEQ Identifiers: B2232-B2240 of Table 3B. In some embodiments, the PEgRNA comprises a PBS comprising a sequence selected from the group consisting of SEQ ID NOs. 2949-2955 of Table 3B. In some embodiments, the PEgRNA comprises a PBS comprising a sequence selected from the group consisting of sequences AGGAGACA and AGGAGACAG as disclosed in Table 3B. In some embodiments, the PEgRNA comprises an editing template comprising a sequence selected from the group consisting of SEQ Identifiers: C10671-C10716 of Table 3C. In some embodiments, the PEgRNA comprises an editing template comprising a sequence selected from the group consisting of SEQ ID NOs:14697-14742 of Table 3C. In some embodiments, the prime editing composition further comprises a ngRNA that comprises a spacer comprising a sequence selected from the group consisting of SEQ Identifiers: D1240-D1245, D1248-D1249, D1251-D1257, D1259, and D1284-1291 of Table 3D. In some embodiments, the prime editing composition further comprises a ngRNA that comprises a spacer comprising a sequence selected from the group consisting of SEQ ID NOs. 20873-20878, 20881-20882, 20884-20890, 20892, and 20917-20924 of Table 3D.

In some embodiments, the PEgRNA comprises a spacer comprising a sequence selected from the group consisting of SEQ Identifiers: A570-A571 of Table 3A. In some embodiments, the PEgRNA comprises a spacer comprising a sequence as set forth in SEQ ID NOs. 918-919 of Table 3A. In some embodiments, the PEgRNA comprises a PBS comprising a sequence selected from the group consisting of SEQ Identifiers: B2241-B2249 of Table 3B. In some embodiments, the PEgRNA comprises a PBS comprising a sequence selected from the group consisting of SEQ ID NOs. 2956-2962 of Table 3B. In some embodiments, the PEgRNA comprises a PBS comprising a sequence selected from the group consisting of sequences AGACAGGA and AGACAGGAG as disclosed in Table 3B. In some embodiments, the PEgRNA comprises an editing template comprising a sequence selected from the group consisting of SEQ Identifiers: C10717-C10736 of Table 3C. In some embodiments, the PEgRNA comprises an editing template comprising a sequence selected from the group consisting of SEQ ID NOs:14743-14762 of Table 3C. In some embodiments, the prime editing composition further comprises a ngRNA that comprises a spacer comprising a sequence selected from the group consisting of SEQ Identifiers: D1158-D1164, D1166-D1190, D1228, D1229, D1235, and D1237 of Table 3D. In some embodiments, the prime editing composition further comprises a ngRNA that comprises a spacer comprising a sequence selected from the group consisting of SEQ ID NOs. 20791-20797, 20799-20823, 20861, 20862, 20868, and 20870 of Table 3D.

In some embodiments, the PEgRNA comprises a spacer comprising the sequence of SEQ Identifier: A572 of Table 3A. In some embodiments, the PEgRNA comprises a spacer comprising a sequence as set forth in SEQ ID NO. 920 of Table 3A. In some embodiments, the PEgRNA comprises a PBS comprising a sequence selected from the group consisting of SEQ Identifiers: B2250-B2258 of Table 3B. In some embodiments, the PEgRNA comprises a PBS comprising a sequence selected from the group consisting of SEQ ID NOs. 2963-2969 of Table 3B. In some embodiments, the PEgRNA comprises a PBS comprising a sequence selected from the group consisting of sequences GCATCTCC and GCATCTCCT as disclosed in Table 3B. In some embodiments, the PEgRNA comprises an editing template comprising a sequence selected from the group consisting of SEQ Identifiers: C10737-C10748 of Table 3C. In some embodiments, the PEgRNA comprises an editing template comprising a sequence selected from the group consisting of SEQ ID NOs:14763-14774 of Table 3C. In some embodiments, the prime editing composition further comprises a ngRNA that comprises a spacer comprising a sequence selected from the group consisting of SEQ Identifiers: D1165-D1188, and D1190 of Table 3D. In some embodiments, the prime editing composition further comprises a ngRNA that comprises a spacer comprising a sequence selected from the group consisting of SEQ ID NOs. 20798-20821, and 20823 of Table 3D.

In some embodiments, the PEgRNA comprises a spacer comprising a sequence selected from the group consisting of SEQ Identifiers: A573-A574 of Table 3A. In some embodiments, the PEgRNA comprises a spacer comprising a sequence as set forth in SEQ ID NOs. 921-922 of Table 3A. In some embodiments, the PEgRNA comprises a PBS comprising a sequence selected from the group consisting of SEQ Identifiers: B2259-B2267 of Table 3B. In some embodiments, the PEgRNA comprises a PBS comprising a sequence selected from the group consisting of SEQ ID NOs. 2070-2076 of Table 3B. In some embodiments, the PEgRNA comprises a PBS comprising a sequence selected from the group consisting of sequences GTTTCTGT and GTTTCTGTC as disclosed in Table 3B. In some embodiments, the PEgRNA comprises an editing template comprising a sequence selected from the group consisting of SEQ Identifiers: C10749-C10779 of Table 3C. In some embodiments, the PEgRNA comprises an editing template comprising a sequence selected from the group consisting of SEQ ID NOs:14775-14805 of Table 3C. In some embodiments, the prime editing composition further comprises a ngRNA that comprises a spacer comprising a sequence selected from the group consisting of SEQ Identifiers: D1241-D1245, D1248, D1249, D1252-D1259, D1286, D1287, D1290, D1291, D1292, D1293, D1294, and D1295 of Table 3D. In some embodiments, the prime editing composition further comprises a ngRNA that comprises a spacer comprising a sequence selected from the group consisting of SEQ ID NOs. 20874-20878, 20881, 20882, 20885-20892, 20919, 20920, 20923, 20924, 20925, 20926, 20927, and 20928 of Table 3D.

In some embodiments, the PEgRNA comprises a spacer comprising the sequence of Identifier: A575 of Table 3A. In some embodiments, the PEgRNA comprises a spacer comprising a sequence as set forth in SEQ ID NO. 923 of Table 3A. In some embodiments, the PEgRNA comprises a PBS comprising a sequence selected from the group consisting of SEQ Identifiers: B2268-B2276 of Table 3B. In some embodiments, the PEgRNA comprises a PBS comprising a sequence selected from the group consisting of SEQ ID NOs. 2977-2983 of Table 3B. In some embodiments, the PEgRNA comprises a PBS comprising a sequence selected from the group consisting of sequences TGTTTCTG and TGTTTCTGT as disclosed in Table 3B. In some embodiments, the PEgRNA comprises an editing template comprising a sequence selected from the group consisting of SEQ Identifiers: C10780-C10810 of Table 3C. In some embodiments, the PEgRNA comprises an editing template comprising a sequence selected from the group consisting of SEQ ID NOs:14806-14836 of Table 3C. In some embodiments, the prime editing composition further comprises a ngRNA that comprises a spacer comprising a sequence selected from the group consisting of SEQ Identifiers: D1170-D1190, D1228, D1229, D1237, D1238, D1296, D1297, and D1298 of Table 3D. In some embodiments, the prime editing composition further comprises a ngRNA that comprises a spacer comprising a sequence selected from the group consisting of SEQ ID NOs. 20803-20823, 20861, 20862, 20870, 20871, and 20929-20931 of Table 3D.

In some embodiments, the PEgRNA comprises a spacer comprising the sequence of SEQ Identifier: A576 of Table 3A. In some embodiments, the PEgRNA comprises a spacer comprising a sequence as set forth in SEQ ID NO. 924 of Table 3A. In some embodiments, the PEgRNA comprises a PBS comprising a sequence selected from the group consisting of SEQ Identifiers: B2277-B2285 of Table 3B. In some embodiments, the PEgRNA comprises a PBS comprising a sequence selected from the group consisting of SEQ ID NOs. 2984-2990 of Table 3B. In some embodiments, the PEgRNA comprises a PBS comprising a sequence selected from the group consisting of sequences TGGAGAGT and TGGAGAGTT as disclosed in Table 3B. In some embodiments, the PEgRNA comprises an editing template comprising a sequence selected from the group consisting of SEQ Identifiers: C10811-C10831 of Table 3C. In some embodiments, the PEgRNA comprises an editing template comprising a sequence selected from the group consisting of SEQ ID NOs:14837-14857 of Table 3C. In some embodiments, the prime editing composition further comprises a ngRNA that comprises a spacer comprising a sequence selected from the group consisting of SEQ Identifiers: D1195-D1214, D1268, and D1269 of Table 3D. In some embodiments, the prime editing composition further comprises a ngRNA that comprises a spacer comprising a sequence selected from the group consisting of SEQ ID NOs. 20828-20847, 20901, and 20902 of Table 3D.

In some embodiments, the PEgRNA comprises a spacer comprising a sequence selected from the group consisting of SEQ Identifiers: A546-A547 of Table 3A. In some embodiments, the PEgRNA comprises a spacer comprising a sequence as set forth in SEQ ID NOs. 894-895 of Table 3A. In some embodiments, the PEgRNA comprises a PBS comprising a sequence selected from the group consisting of SEQ Identifiers: B2124-B2132 of Table 3B. In some embodiments, the PEgRNA comprises a PBS comprising a sequence selected from the group consisting of SEQ ID NOs. 2865-2871 of Table 3B. In some embodiments, the PEgRNA comprises a PBS comprising a sequence selected from the group consisting of sequences AACAGACT and AACAGACTG as disclosed in Table 3B. In some embodiments, the PEgRNA comprises an editing template comprising a sequence selected from the group consisting of SEQ Identifiers: C10832-C10889 of Table 3C. In some embodiments, the PEgRNA comprises an editing template comprising a sequence selected from the group consisting of SEQ ID NOs:14858-14915 of Table 3C. In some embodiments, the prime editing composition further comprises a ngRNA that comprises a spacer comprising a sequence selected from the group consisting of SEQ Identifiers: D1139-D1156 of Table 3D. In some embodiments, the prime editing composition further comprises a ngRNA that comprises a spacer comprising a sequence selected from the group consisting of SEQ ID NOs. 20772-20789 of Table 3D.

In some embodiments, the PEgRNA comprises a spacer comprising a sequence selected from the group consisting of SEQ Identifiers: A548-A549 of Table 3A. In some embodiments, the PEgRNA comprises a spacer comprising a sequence as set forth in SEQ ID NOs. 896-897 of Table 3A. In some embodiments, the PEgRNA comprises a PBS comprising a sequence selected from the group consisting of SEQ Identifiers: B2133-B2141 of Table 3B. In some embodiments, the PEgRNA comprises a PBS comprising a sequence selected from the group consisting of SEQ ID NOs. 2872-2878 of Table 3B. In some embodiments, the PEgRNA comprises a PBS comprising a sequence selected from the group consisting of sequences TAATGAGA and TAATGAGAA as disclosed in Table 3B. In some embodiments, the PEgRNA comprises an editing template comprising the sequence of SEQ Identifier: C10890 of Table 3C. In some embodiments, the PEgRNA comprises an editing template comprising a sequence selected from the group consisting of SEQ ID NOs:14916- of Table 3C. In some embodiments, the prime editing composition further comprises a ngRNA that comprises a spacer comprising a sequence selected from the group consisting of SEQ Identifiers: D1157-D1188 of Table 3D. In some embodiments, the prime editing composition further comprises a ngRNA that comprises a spacer comprising a sequence selected from the group consisting of SEQ ID NOs. 20790-20821 of Table 3D.

In some embodiments, the PEgRNA comprises a spacer comprising a sequence selected from the group consisting of SEQ Identifiers: A550-A551 of Table 3A. In some embodiments, the PEgRNA comprises a spacer comprising a sequence as set forth in SEQ ID NOs. 898-899 of Table 3A. In some embodiments, the PEgRNA comprises a PBS comprising a sequence selected from the group consisting of SEQ Identifiers: B2142-B2150 of Table 3B. In some embodiments, the PEgRNA comprises a PBS comprising a sequence selected from the group consisting of SEQ ID NOs. 2879-2885 of Table 3B. In some embodiments, the PEgRNA comprises a PBS comprising a sequence selected from the group consisting of sequences TCTAATGA and TCTAATGAG as disclosed in Table 3B. In some embodiments, the PEgRNA comprises an editing template comprising a sequence selected from the group consisting of SEQ Identifiers: C10891-C10893 of Table 3C. In some embodiments, the PEgRNA comprises an editing template comprising a sequence selected from the group consisting of SEQ ID NOs:14917-14919 of Table 3C. In some embodiments, the prime editing composition further comprises a ngRNA that comprises a spacer comprising a sequence selected from the group consisting of SEQ Identifiers: D1157-D1190 of Table 3D. In some embodiments, the prime editing composition further comprises a ngRNA that comprises a spacer comprising a sequence selected from the group consisting of SEQ ID NOs. 20790-20823 of Table 3D.

In some embodiments, the PEgRNA comprises a spacer comprising a sequence selected from the group consisting of SEQ Identifiers: A552-A553 of Table 3A. In some embodiments, the PEgRNA comprises a spacer comprising a sequence as set forth in SEQ ID NOs. 900-901 of Table 3A. In some embodiments, the PEgRNA comprises a PBS comprising a sequence selected from the group consisting of SEQ Identifiers: B2151-B2159 of Table 3B. In some embodiments, the PEgRNA comprises a PBS comprising a sequence selected from the group consisting of SEQ ID NOs. 2886-2892 of Table 3B. In some embodiments, the PEgRNA comprises a PBS comprising a sequence selected from the group consisting of sequences AAACAGAC and AAACAGACT as disclosed in Table 3B. In some embodiments, the PEgRNA comprises an editing template comprising a sequence selected from the group consisting of SEQ Identifiers: C10894-C10923 of Table 3C. In some embodiments, the PEgRNA comprises an editing template comprising a sequence selected from the group consisting of SEQ ID NOs:14920-14949 of Table 3C. In some embodiments, the prime editing composition further comprises a ngRNA that comprises a spacer comprising a sequence selected from the group consisting of SEQ Identifiers: D1194-D1215, D1216-D1227, D1299, D1300, D1301, and D1302 of Table 3D. In some embodiments, the prime editing composition further comprises a ngRNA that comprises a spacer comprising a sequence selected from the group consisting of SEQ ID NOs. 20827-20848, 20849-20860, 20932, 20933, 20934, and 20935 of Table 3D.

In some embodiments, the PEgRNA comprises a spacer comprising a sequence selected from the group consisting of SEQ Identifiers: A554-A555 of Table 3A. In some embodiments, the PEgRNA comprises a spacer comprising a sequence as set forth in SEQ ID NOs. 902-903 of Table 3A. In some embodiments, the PEgRNA comprises a PBS comprising a sequence selected from the group consisting of SEQ Identifiers: B2160-B2168 of Table 3B. In some embodiments, the PEgRNA comprises a PBS comprising a sequence selected from the group consisting of SEQ ID NOs. 2893-2899 of Table 3B. In some embodiments, the PEgRNA comprises a PBS comprising a sequence selected from the group consisting of sequences GACAGGAG and GACAGGAGC as disclosed in Table 3B. In some embodiments, the PEgRNA comprises an editing template comprising a sequence selected from the group consisting of SEQ Identifiers: C10924-C10942 of Table 3C. In some embodiments, the PEgRNA comprises an editing template comprising a sequence selected from the group consisting of SEQ ID NOs:14950-14968 of Table 3C. In some embodiments, the prime editing composition further comprises a ngRNA that comprises a spacer comprising a sequence selected from the group consisting of SEQ Identifiers: D1166-D1190, and D1228-D1234 of Table 3D. In some embodiments, the prime editing composition further comprises a ngRNA that comprises a spacer comprising a sequence selected from the group consisting of SEQ ID NOs. 20799-20823, and 20861-20867 of Table 3D.

In some embodiments, the PEgRNA comprises a spacer comprising a sequence selected from the group consisting of SEQ Identifiers: A556-A557 of Table 3A. In some embodiments, the PEgRNA comprises a spacer comprising a sequence as set forth in SEQ ID NOs. 904-905 of Table 3A. In some embodiments, the PEgRNA comprises a PBS comprising a sequence selected from the group consisting of SEQ Identifiers: B2169-B2177 of Table 3B. In some embodiments, the PEgRNA comprises a PBS comprising a sequence selected from the group consisting of SEQ ID NOs. 2900-2906 of Table 3B. In some embodiments, the PEgRNA comprises a PBS comprising a sequence selected from the group consisting of sequences CAGGAGAC and CAGGAGACA as disclosed in Table 3B. In some embodiments, the PEgRNA comprises an editing template comprising a sequence selected from the group consisting of SEQ Identifiers: C10943-C10966 of Table 3C. In some embodiments, the PEgRNA comprises an editing template comprising a sequence selected from the group consisting of SEQ ID NOs:14969-14992 of Table 3C. In some embodiments, the prime editing composition further comprises a ngRNA that comprises a spacer comprising a sequence selected from the group consisting of SEQ Identifiers: D1158-D1164, D1167-D1190, D1228, D1229, D1235, D1237, D1303, and D1304 of Table 3D. In some embodiments, the prime editing composition further comprises a ngRNA that comprises a spacer comprising a sequence selected from the group consisting of SEQ ID NOs. 20791-20797, 20800-20823, 20861, 20862, 20868, 20870, 20936, and 20937 of Table 3D.

In some embodiments, the PEgRNA comprises a spacer comprising a sequence selected from the group consisting of SEQ Identifiers: A558-A559 of Table 3A. In some embodiments, the PEgRNA comprises a spacer comprising a sequence as set forth in SEQ ID NOs. 906-907 of Table 3A. In some embodiments, the PEgRNA comprises a PBS comprising a sequence selected from the group consisting of SEQ Identifiers: B2178-B2186 of Table 3B. In some embodiments, the PEgRNA comprises a PBS comprising a sequence selected from the group consisting of SEQ ID NOs. 2907-2913 of Table 3B. In some embodiments, the PEgRNA comprises a PBS comprising a sequence selected from the group consisting of sequences CTAATGAG and CTAATGAGA as disclosed in Table 3B. In some embodiments, the PEgRNA comprises an editing template comprising a sequence selected from the group consisting of SEQ Identifiers: C10967-C10970 of Table 3C. In some embodiments, the PEgRNA comprises an editing template comprising a sequence selected from the group consisting of SEQ ID NOs:14993-14996 of Table 3C. In some embodiments, the prime editing composition further comprises a ngRNA that comprises a spacer comprising a sequence selected from the group consisting of SEQ Identifiers: D1239-D1259 of Table 3D. In some embodiments, the prime editing composition further comprises a ngRNA that comprises a spacer comprising a sequence selected from the group consisting of SEQ ID NOs. 20872-20892 of Table 3D.

In some embodiments, the PEgRNA comprises a spacer comprising the sequence of SEQ Identifier: A560 of Table 3A. In some embodiments, the PEgRNA comprises a spacer comprising a sequence as set forth in SEQ ID NO. 908 of Table 3A. In some embodiments, the PEgRNA comprises a PBS comprising a sequence selected from the group consisting of SEQ Identifiers: B2187-B2195 of Table 3B. In some embodiments, the PEgRNA comprises a PBS comprising a sequence selected from the group consisting of SEQ ID NOs. 2914-2920 of Table 3B. In some embodiments, the PEgRNA comprises a PBS comprising a sequence selected from the group consisting of sequences CCTTCTAA and CCTTCTAAT as disclosed in Table 3B. In some embodiments, the PEgRNA comprises an editing template comprising a sequence selected from the group consisting of SEQ Identifiers: C10971-C10976 of Table 3C. In some embodiments, the PEgRNA comprises an editing template comprising a sequence selected from the group consisting of SEQ ID NOs:14997-15002 of Table 3C. In some embodiments, the prime editing composition further comprises a ngRNA that comprises a spacer comprising a sequence selected from the group consisting of SEQ Identifiers: D1165-D1188, and D1190 of Table 3D. In some embodiments, the prime editing composition further comprises a ngRNA that comprises a spacer comprising a sequence selected from the group consisting of SEQ ID NOs. 20798-20821, and 20823 of Table 3D.

In some embodiments, the PEgRNA comprises a spacer comprising the sequence of SEQ Identifier: A561 of Table 3A. In some embodiments, the PEgRNA comprises a spacer comprising a sequence as set forth in SEQ ID NO. 909 of Table 3A. In some embodiments, the PEgRNA comprises a PBS comprising a sequence selected from the group consisting of SEQ Identifiers: B2196-B2204 of Table 3B. In some embodiments, the PEgRNA comprises a PBS comprising a sequence selected from the group consisting of SEQ ID NOs. 2921-2927 of Table 3B. In some embodiments, the PEgRNA comprises a PBS comprising a sequence selected from the group consisting of sequences TTTTAAAC and TTTTAAACA as disclosed in Table 3B. In some embodiments, the PEgRNA comprises an editing template comprising a sequence selected from the group consisting of SEQ Identifiers: C10977-C11007 of Table 3C. In some embodiments, the PEgRNA comprises an editing template comprising a sequence selected from the group consisting of SEQ ID NOs:15003-15033 of Table 3C. In some embodiments, the prime editing composition further comprises a ngRNA that comprises a spacer comprising a sequence selected from the group consisting of SEQ Identifiers: D1194-D1213, D1299, D1300, and D1301 of Table 3D. In some embodiments, the prime editing composition further comprises a ngRNA that comprises a spacer comprising a sequence selected from the group consisting of SEQ ID NOs. 20827-20846, 20932, 20933, and 20934 of Table 3D.

In some embodiments, the PEgRNA comprises a spacer comprising a sequence selected from the group consisting of SEQ Identifier: A562-A563 of Table 3A. In some embodiments, the PEgRNA comprises a spacer comprising a sequence as set forth in SEQ ID NOs. 910-911 of Table 3A. In some embodiments, the PEgRNA comprises a PBS comprising a sequence selected from the group consisting of SEQ Identifiers: B2205-B2213 of Table 3B. In some embodiments, the PEgRNA comprises a PBS comprising a sequence selected from the group consisting of SEQ ID NOs. 2928-2934 of Table 3B. In some embodiments, the PEgRNA comprises a PBS comprising a sequence selected from the group consisting of sequences AGTTTGGG and AGTTTGGGG as disclosed in Table 3B. In some embodiments, the PEgRNA comprises an editing template comprising a sequence selected from the group consisting of SEQ Identifiers: C11008-C11041 of Table 3C. In some embodiments, the PEgRNA comprises an editing template comprising a sequence selected from the group consisting of SEQ ID NOs:15034-15067 of Table 3C. In some embodiments, the prime editing composition further comprises a ngRNA that comprises a spacer comprising a sequence selected from the group consisting of SEQ Identifiers: D1141-D1145, D1150-D1154, and D1260-D1267 of Table 3D. In some embodiments, the prime editing composition further comprises a ngRNA that comprises a spacer comprising a sequence selected from the group consisting of SEQ ID NOs. 20774-20778, 20783-20788, and 20893-20900 of Table 3D.

In some embodiments, the PEgRNA comprises a spacer comprising the sequence of SEQ Identifier: A564 of Table 3A. In some embodiments, the PEgRNA comprises a spacer comprising a sequence as set forth in SEQ ID NO. 912 of Table 3A. In some embodiments, the PEgRNA comprises a PBS comprising a sequence selected from the group consisting of SEQ Identifiers: B2214-B2222 of Table 3B. In some embodiments, the PEgRNA comprises a PBS comprising a sequence selected from the group consisting of SEQ ID NOs. 2935-2941 of Table 3B. In some embodiments, the PEgRNA comprises a PBS comprising a sequence selected from the group consisting of sequences GAGTTTGG and GAGTTTGGG as disclosed in Table 3B. In some embodiments, the PEgRNA comprises an editing template comprising a sequence selected from the group consisting of SEQ Identifiers: C11042-C11059 of Table 3C. In some embodiments, the PEgRNA comprises an editing template comprising a sequence selected from the group consisting of SEQ ID NOs:15068-15085 of Table 3C. In some embodiments, the prime editing composition further comprises a ngRNA that comprises a spacer comprising a sequence selected from the group consisting of SEQ Identifiers: D1196-D1214, and D1268-D1270 of Table 3D. In some embodiments, the prime editing composition further comprises a ngRNA that comprises a spacer comprising a sequence selected from the group consisting of SEQ ID NOs. 20829-20847, and 20901-20903 of Table 3D.

In some embodiments, the PEgRNA comprises a spacer comprising a sequence selected from the group consisting of SEQ Identifier: A565-A567 of Table 3A. In some embodiments, the PEgRNA comprises a spacer comprising a sequence as set forth in SEQ ID NOs. 913-915 of Table 3A. In some embodiments, the PEgRNA comprises a PBS comprising a sequence selected from the group consisting of SEQ Identifiers: B2223-B2231 of Table 3B. In some embodiments, the PEgRNA comprises a PBS comprising a sequence selected from the group consisting of SEQ ID NOs. 2942-2948 of Table 3B. In some embodiments, the PEgRNA comprises a PBS comprising a sequence selected from the group consisting of sequences ACAGGAGC and ACAGGAGCA as disclosed in Table 3B. In some embodiments, the PEgRNA comprises an editing template comprising a sequence selected from the group consisting of SEQ Identifiers: C11060-C11077 of Table 3C. In some embodiments, the PEgRNA comprises an editing template comprising a sequence selected from the group consisting of SEQ ID NOs:15086-15103 of Table 3C. In some embodiments, the prime editing composition further comprises a ngRNA that comprises a spacer comprising a sequence selected from the group consisting of SEQ Identifiers: D1244, D1248, and D1271-D1283 of Table 3D. In some embodiments, the prime editing composition further comprises a ngRNA that comprises a spacer comprising a sequence selected from the group consisting of SEQ ID NOs. 20877, 20881, and 20904-20916 of Table 3D.

In some embodiments, the PEgRNA comprises a spacer comprising a sequence selected from the group consisting of SEQ Identifiers: A568-A569 of Table 3A. In some embodiments, the PEgRNA comprises a spacer comprising a sequence as set forth in SEQ ID NOs. 916-917 of Table 3A. In some embodiments, the PEgRNA comprises a PBS comprising a sequence selected from the group consisting of SEQ Identifiers: B2232-B2240 of Table 3B. In some embodiments, the PEgRNA comprises a PBS comprising a sequence selected from the group consisting of SEQ ID NOs. 2949-2955 of Table 3B. In some embodiments, the PEgRNA comprises a PBS comprising a sequence selected from the group consisting of sequences AGGAGACA and AGGAGACAG as disclosed in Table 3B. In some embodiments, the PEgRNA comprises an editing template comprising a sequence selected from the group consisting of SEQ Identifiers: C11078-C11123 of Table 3C. In some embodiments, the PEgRNA comprises an editing template comprising a sequence selected from the group consisting of SEQ ID NOs:15104-15149 of Table 3C. In some embodiments, the prime editing composition further comprises a ngRNA that comprises a spacer comprising a sequence selected from the group consisting of SEQ Identifiers: D1240-D1245, D1248-D1257, D1259, and D1284-D1291 of Table 3D. In some embodiments, the prime editing composition further comprises a ngRNA that comprises a spacer comprising a sequence selected from the group consisting of SEQ ID NOs. 20873-20878, 20881-20890, 20892, and 20917-20924 of Table 3D.

In some embodiments, the PEgRNA comprises a spacer comprising a sequence selected from the group consisting of SEQ Identifiers: A570-A571 of Table 3A. In some embodiments, the PEgRNA comprises a spacer comprising a sequence as set forth in SEQ ID NOs. 918-919 of Table 3A. In some embodiments, the PEgRNA comprises a PBS comprising a sequence selected from the group consisting of SEQ Identifiers: B2241-B2249 of Table 3B. In some embodiments, the PEgRNA comprises a PBS comprising a sequence selected from the group consisting of SEQ ID NOs. 2956-2962 of Table 3B. In some embodiments, the PEgRNA comprises a PBS comprising a sequence selected from the group consisting of sequences AGACAGGA and AGACAGGAG as disclosed in Table 3B. In some embodiments, the PEgRNA comprises an editing template comprising a sequence selected from the group consisting of SEQ Identifiers: C11124-C11143 of Table 3C. In some embodiments, the PEgRNA comprises an editing template comprising a sequence selected from the group consisting of SEQ ID NOs:15150-15169 of Table 3C. In some embodiments, the prime editing composition further comprises a ngRNA that comprises a spacer comprising a sequence selected from the group consisting of SEQ Identifiers: D1158-D1164, D1166-D1190, D1228, D1229, D1235, and D1237 of Table 3D. In some embodiments, the prime editing composition further comprises a ngRNA that comprises a spacer comprising a sequence selected from the group consisting of SEQ ID NOs. 20791-20797, 20799-20823, 20861, 20862, 20868, and 20870 of Table 3D.

In some embodiments, the PEgRNA comprises a spacer comprising the sequence of SEQ Identifier: A572 of Table 3A. In some embodiments, the PEgRNA comprises a spacer comprising a sequence as set forth in SEQ ID NO. 920 of Table 3A. In some embodiments, the PEgRNA comprises a PBS comprising a sequence selected from the group consisting of SEQ Identifiers: B2250-B2258 of Table 3B. In some embodiments, the PEgRNA comprises a PBS comprising a sequence selected from the group consisting of SEQ ID NOs. 2963-2969 of Table 3B. In some embodiments, the PEgRNA comprises a PBS comprising a sequence selected from the group consisting of sequences GCATCTCC and GCATCTCCT as disclosed in Table 3B. In some embodiments, the PEgRNA comprises an editing template comprising a sequence selected from the group consisting of SEQ Identifiers: C11144-C11155 of Table 3C. In some embodiments, the PEgRNA comprises an editing template comprising a sequence selected from the group consisting of SEQ ID NOs:15170-15181 of Table 3C. In some embodiments, the prime editing composition further comprises a ngRNA that comprises a spacer comprising a sequence selected from the group consisting of SEQ Identifiers: D1165-D1188, D1190 of Table 3D. In some embodiments, the prime editing composition further comprises a ngRNA that comprises a spacer comprising a sequence selected from the group consisting of SEQ ID NOs. 20798-20821, and 20823 of Table 3D.

In some embodiments, the PEgRNA comprises a spacer comprising the sequence of SEQ Identifier: A576 of Table 3A. In some embodiments, the PEgRNA comprises a spacer comprising a sequence as set forth in SEQ ID NO. 924 of Table 3A. In some embodiments, the PEgRNA comprises a PBS comprising a sequence selected from the group consisting of SEQ Identifiers: B2277-B2285 of Table 3B. In some embodiments, the PEgRNA comprises a PBS comprising a sequence selected from the group consisting of SEQ ID NOs. 2984-2990 of Table 3B. In some embodiments, the PEgRNA comprises a PBS comprising a sequence selected from the group consisting of sequences TGGAGAGT and TGGAGAGTT as disclosed in Table 3B. In some embodiments, the PEgRNA comprises an editing template comprising a sequence selected from the group consisting of SEQ Identifiers: C11156-C11177 of Table 3C. In some embodiments, the PEgRNA comprises an editing template comprising a sequence selected from the group consisting of SEQ ID NOs:15182-15203 of Table 3C. In some embodiments, the prime editing composition further comprises a ngRNA that comprises a spacer comprising a sequence selected from the group consisting of SEQ Identifiers: D1195-D1214, D1268, and D1269 of Table 3D. In some embodiments, the prime editing composition further comprises a ngRNA that comprises a spacer comprising a sequence selected from the group consisting of SEQ ID NOs. 20828-20847, 20910, and 20911 of Table 3D.

In some embodiments, the editing target sequence is in exon 21 of the CFTR gene.

In some embodiments, the editing target sequence or the complementary sequence on the CFTR gene thereof comprises a mutation that encodes an amino acid substitution D1152H, N1148K, V1153E and/or D 1154Y relative to a wild type CFTR polypeptide set forth in SEQ ID NO: 19, and wherein the editing template encodes an intended nucleotide edit that corrects the mutation. In some embodiments, the editing target sequence or the complementary sequence on the CFTR gene thereof comprises a G→C mutation (c.3454G>C) at position 117,614,699 a C→A mutation (c.3444C>A) at position 117,614,689, a deletion of C (c.3444delC) at position 117,614,689, a deletion of T (c.3445delT) at position 117,614,690, a T→A mutation (c.3458T>A) at position 117,614,703, and/or a G→T mutation (c.3460G>T) at position 117,614,705 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide.

In some embodiments, the editing template comprises a wild type CFTR gene sequence. Accordingly, in some embodiments, the newly synthesized single stranded DNA encoded by the editing template is capable of correcting the c.3454G>C mutation, the c.3444C>A mutation, the c.3444delC mutation, the c.3445delT mutation, the c.3458T>A mutation, and/or the c.3460G>T mutation in the target CFTR gene. In some embodiments, the newly synthesized single stranded DNA encoded by the editing template is capable of correcting all of the c.3454G>C mutation, the c.3444C>A mutation, the c.3444delC mutation, the c.3445delT mutation, the c.3458T>A mutation, and the c.3460G>T mutation in the target CFTR gene. In some embodiments, two or more of the c.3454G>C mutation, the c.3444C>A mutation, the c.3444delC mutation, the c.3445delT mutation, the c.3458T>A mutation, and the c.3460G>T mutation are located on the same CFTR gene. In some embodiments, the newly synthesized single stranded DNA encoded by the editing template is capable of correcting the c.3454G>C mutation, the c.3444C>A mutation, the c.3444delC mutation, the c.3445delT mutation, the c.3458T>A mutation, and the c.3460G>T mutation that are located on different CFTR genes. For example, in some embodiments, a subject may comprise two copies of CFTR genes, each comprising a different mutation selected from the group consisting of the c.3454G>C mutation, the c.3444C>A mutation, the c.3444delC mutation, the c.3445delT mutation, the c.3458T>A mutation, and the c.3460G>T mutation, all of which may be corrected by an editing template of a single PEgRNA. In some embodiments, the editing template is capable of correcting one or more, or all of the c.3454G>C mutation, the c.3444C>A mutation, the c.3444delC mutation, the c.3445delT mutation, the c.3458T>A mutation, and the c.3460G>T mutation that are in different subjects, wherein each subject may have one or more of the mutations.

In some embodiments, the PEgRNA comprises a spacer comprising a sequence selected from the group consisting of SEQ Identifiers: A577-A578 of Table 3A. In some embodiments, the PEgRNA comprises a spacer comprising a sequence as set forth in SEQ ID NOs. 925-926 of Table 3A. In some embodiments, the PEgRNA comprises a PBS comprising a sequence selected from the group consisting of SEQ Identifiers: B2286-B2294 of Table 3B. In some embodiments, the PEgRNA comprises a PBS comprising a sequence selected from the group consisting of SEQ ID NOs. 2991-2997 of Table 3B. In some embodiments, the PEgRNA comprises a PBS comprising a sequence selected from the group consisting of sequences CATCTTTT and CATCTTTTT as disclosed in Table 3B. In some embodiments, the PEgRNA comprises an editing template comprising a sequence selected from the group consisting of SEQ Identifiers: C11178-C11191 of Table 3C. In some embodiments, the PEgRNA comprises an editing template comprising a sequence selected from the group consisting of SEQ ID NOs:15204-15217 of Table 3C. In some embodiments, the prime editing composition further comprises a ngRNA that comprises a spacer comprising a sequence selected from the group consisting of SEQ Identifiers: D1305-D1334 of Table 3D. In some embodiments, the prime editing composition further comprises a ngRNA that comprises a spacer comprising a sequence selected from the group consisting of SEQ ID NOs. 20938-20967 of Table 3D.

In some embodiments, the PEgRNA comprises a spacer comprising a sequence selected from the group consisting of SEQ Identifiers: A579-A580 of Table 3A. In some embodiments, the PEgRNA comprises a spacer comprising a sequence as set forth in SEQ ID NOs. 927-928 of Table 3A. In some embodiments, the PEgRNA comprises a PBS comprising a sequence selected from the group consisting of SEQ Identifiers: B2295-B2303 of Table 3B. In some embodiments, the PEgRNA comprises a PBS comprising a sequence selected from the group consisting of SEQ ID NOs. 2998-3004 of Table 3B. In some embodiments, the PEgRNA comprises a PBS comprising a sequence selected from the group consisting of sequences AAGTCTTA and AAGTCTTAT as disclosed in Table 3B. In some embodiments, the PEgRNA comprises an editing template comprising a sequence selected from the group consisting of SEQ Identifiers: C11192-C11237 of Table 3C. In some embodiments, the PEgRNA comprises an editing template comprising a sequence selected from the group consisting of SEQ ID NOs:15218-15263 of Table 3C. In some embodiments, the prime editing composition further comprises a ngRNA that comprises a spacer comprising a sequence selected from the group consisting of SEQ Identifiers: D1335-D1354 of Table 3D. In some embodiments, the prime editing composition further comprises a ngRNA that comprises a spacer comprising a sequence selected from the group consisting of SEQ ID NOs. 20968-20987 of Table 3D.

In some embodiments, the PEgRNA comprises a spacer comprising the sequence of SEQ Identifier: A581 of Table 3A. In some embodiments, the PEgRNA comprises a spacer comprising a sequence as set forth in SEQ ID NO. 929 of Table 3A. In some embodiments, the PEgRNA comprises a PBS comprising a sequence selected from the group consisting of SEQ Identifiers: B2304-B2312 of Table 3B. In some embodiments, the PEgRNA comprises a PBS comprising a sequence selected from the group consisting of SEQ ID NOs. of Table 3B. In some embodiments, the PEgRNA comprises a PBS comprising a sequence selected from the group consisting of sequences as disclosed in Table 3B. In some embodiments, the PEgRNA comprises an editing template comprising a sequence selected from the group consisting of SEQ Identifiers: C11238-C11261 of Table 3C. In some embodiments, the PEgRNA comprises an editing template comprising a sequence selected from the group consisting of SEQ ID NOs:15264-15287 of Table 3C. In some embodiments, the prime editing composition further comprises a ngRNA that comprises a spacer comprising a sequence selected from the group consisting of SEQ Identifiers: D1315-D1330 of Table 3D. In some embodiments, the prime editing composition further comprises a ngRNA that comprises a spacer comprising a sequence selected from the group consisting of SEQ ID NOs. 20948-20963 of Table 3D.

In some embodiments, the PEgRNA comprises a spacer comprising a sequence selected from the group consisting of SEQ Identifier: A582-A584 of Table 3A. In some embodiments, the PEgRNA comprises a spacer comprising a sequence as set forth in SEQ ID NOs. 930-932 of Table 3A. In some embodiments, the PEgRNA comprises a PBS comprising a sequence selected from the group consisting of SEQ Identifiers: B2313-B2321 of Table 3B. In some embodiments, the PEgRNA comprises a PBS comprising a sequence selected from the group consisting of SEQ ID NOs. 3012-3018 of Table 3B. In some embodiments, the PEgRNA comprises a PBS comprising a sequence selected from the group consisting of sequences AATGTACT and AATGTACTC as disclosed in Table 3B. In some embodiments, the PEgRNA comprises an editing template comprising a sequence selected from the group consisting of SEQ Identifiers: C11262-C11275 of Table 3C. In some embodiments, the PEgRNA comprises an editing template comprising a sequence selected from the group consisting of SEQ ID NOs:15288-15301 of Table 3C. In some embodiments, the prime editing composition further comprises a ngRNA that comprises a spacer comprising a sequence selected from the group consisting of SEQ Identifiers: D1355-D1360 of Table 3D. In some embodiments, the prime editing composition further comprises a ngRNA that comprises a spacer comprising a sequence selected from the group consisting of SEQ ID NOs. 20988-20993 of Table 3D.

In some embodiments, the PEgRNA comprises a spacer comprising a sequence selected from the group consisting of SEQ Identifiers: A585-A586 of Table 3A. In some embodiments, the PEgRNA comprises a spacer comprising a sequence as set forth in SEQ ID NOs. 933-934 of Table 3A. In some embodiments, the PEgRNA comprises a PBS comprising a sequence selected from the group consisting of SEQ Identifiers: B2322-B2330 of Table 3B. In some embodiments, the PEgRNA comprises a PBS comprising a sequence selected from the group consisting of SEQ ID NOs. 3019-3025 of Table 3B. In some embodiments, the PEgRNA comprises a PBS comprising a sequence selected from the group consisting of sequences TATTCATG and TATTCATGG as disclosed in Table 3B. In some embodiments, the PEgRNA comprises an editing template comprising the sequence of SEQ Identifier: C11276 of Table 3C. In some embodiments, the PEgRNA comprises an editing template comprising a sequence selected from the group consisting of SEQ ID NOs:15302 of Table 3C. In some embodiments, the prime editing composition further comprises a ngRNA that comprises a spacer comprising a sequence selected from the group consisting of SEQ Identifiers: D1361-D1380 of Table 3D. In some embodiments, the prime editing composition further comprises a ngRNA that comprises a spacer comprising a sequence selected from the group consisting of SEQ ID NOs. 20994-21013 of Table 3D.

In some embodiments, the PEgRNA comprises a spacer comprising a sequence selected from the group consisting of SEQ Identifiers: A587-A588 of Table 3A. In some embodiments, the PEgRNA comprises a spacer comprising a sequence as set forth in SEQ ID NOs. 935-936 of Table 3A. In some embodiments, the PEgRNA comprises a PBS comprising a sequence selected from the group consisting of SEQ Identifiers: B2331-B2339 of Table 3B. In some embodiments, the PEgRNA comprises a PBS comprising a sequence selected from the group consisting of SEQ ID NOs. 3026-3032 of Table 3B. In some embodiments, the PEgRNA comprises a PBS comprising a sequence selected from the group consisting of sequences GCAATGTA and GCAATGTAC as disclosed in Table 3B. In some embodiments, the PEgRNA comprises an editing template comprising a sequence selected from the group consisting of SEQ Identifiers: C11277-C11292 of Table 3C. In some embodiments, the PEgRNA comprises an editing template comprising a sequence selected from the group consisting of SEQ ID NOs:15303-15318 of Table 3C. In some embodiments, the prime editing composition further comprises a ngRNA that comprises a spacer comprising a sequence selected from the group consisting of SEQ Identifiers: D1367-D1384 of Table 3D. In some embodiments, the prime editing composition further comprises a ngRNA that comprises a spacer comprising a sequence selected from the group consisting of SEQ ID NOs. 21000-21017 of Table 3D.

In some embodiments, the PEgRNA comprises a spacer comprising the sequence of SEQ Identifier: A589 of Table 3A. In some embodiments, the PEgRNA comprises a spacer comprising a sequence as set forth in SEQ ID NO. 937 of Table 3A. In some embodiments, the PEgRNA comprises a PBS comprising a sequence selected from the group consisting of SEQ Identifiers: B2340-B2348 of Table 3B. In some embodiments, the PEgRNA comprises a PBS comprising a sequence selected from the group consisting of SEQ ID NOs. 3033-3039 of Table 3B. In some embodiments, the PEgRNA comprises a PBS comprising a sequence selected from the group consisting of sequences TGCAATGT and TGCAATGTA as disclosed in Table 3B. In some embodiments, the PEgRNA comprises an editing template comprising a sequence selected from the group consisting of SEQ Identifiers: C11293-C11309 of Table 3C. In some embodiments, the PEgRNA comprises an editing template comprising a sequence selected from the group consisting of SEQ ID NOs:15319-15335 of Table 3C. In some embodiments, the prime editing composition further comprises a ngRNA that comprises a spacer comprising a sequence selected from the group consisting of SEQ Identifiers: D1367-D1380, D1383, and D1384 of Table 3D. In some embodiments, the prime editing composition further comprises a ngRNA that comprises a spacer comprising a sequence selected from the group consisting of SEQ ID NOs. 21000-21013, 21016, and 21017 of Table 3D.

In some embodiments, the PEgRNA comprises a spacer comprising the sequence of SEQ Identifier: A590 of Table 3A. In some embodiments, the PEgRNA comprises a spacer comprising a sequence as set forth in SEQ ID NO. 938 of Table 3A. In some embodiments, the PEgRNA comprises a PBS comprising a sequence selected from the group consisting of SEQ Identifiers: B2349-B2357 of Table 3B. In some embodiments, the PEgRNA comprises a PBS comprising a sequence selected from the group consisting of SEQ ID NOs. 3040-3046 of Table 3B. In some embodiments, the PEgRNA comprises a PBS comprising a sequence selected from the group consisting of sequences CACTGCAA and CACTGCAAT as disclosed in Table 3B. In some embodiments, the PEgRNA comprises an editing template comprising a sequence selected from the group consisting of SEQ Identifiers: C11310-Cl1329 of Table 3C. In some embodiments, the PEgRNA comprises an editing template comprising a sequence selected from the group consisting of SEQ ID NOs:15336-15355 of Table 3C. In some embodiments, the prime editing composition further comprises a ngRNA that comprises a spacer comprising a sequence selected from the group consisting of SEQ Identifiers D1367-D1380, D1383, and D1384 of Table 3D. In some embodiments, the prime editing composition further comprises a ngRNA that comprises a spacer comprising a sequence selected from the group consisting of SEQ ID NOs. 21000-21013, 21016, and 21017 of Table 3D.

In some embodiments, the PEgRNA comprises a spacer comprising the sequence of SEQ ID Identifier: A591 of Table 3A. In some embodiments, the PEgRNA comprises a spacer comprising a sequence as set forth in SEQ ID NO. 939 of Table 3A. In some embodiments, the PEgRNA comprises a PBS comprising a sequence selected from the group consisting of SEQ Identifiers: B2358-B2366 of Table 3B. In some embodiments, the PEgRNA comprises a PBS comprising a sequence selected from the group consisting of SEQ ID NOs. 3047-3053 of Table 3B. In some embodiments, the PEgRNA comprises a PBS comprising a sequence selected from the group consisting of sequences TACTCATG and TACTCATGA as disclosed in Table 3B. In some embodiments, the PEgRNA comprises an editing template comprising a sequence selected from the group consisting of SEQ Identifiers: C11330-C11339 of Table 3C. In some embodiments, the PEgRNA comprises an editing template comprising a sequence selected from the group consisting of SEQ ID NOs:15356-15365 of Table 3C. In some embodiments, the prime editing composition further comprises a ngRNA that comprises a spacer comprising a sequence selected from the group consisting of SEQ Identifiers: D1367-D1380and D1383 of Table 3D. In some embodiments, the prime editing composition further comprises a ngRNA that comprises a spacer comprising a sequence selected from the group consisting of SEQ ID NOs. 21000-21013, and 21016 of Table 3D.

In some embodiments, the PEgRNA comprises a spacer comprising a sequence selected from the group consisting of SEQ Identifiers: A592-A594 of Table 3A. In some embodiments, the PEgRNA comprises a spacer comprising a sequence as set forth in SEQ ID NOs. 940-942 of Table 3A. In some embodiments, the PEgRNA comprises a PBS comprising a sequence selected from the group consisting of SEQ Identifiers: B2367-B2375 of Table 3B. In some embodiments, the PEgRNA comprises a PBS comprising a sequence selected from the group consisting of SEQ ID NOs. 3054-3060 of Table 3B. In some embodiments, the PEgRNA comprises a PBS comprising a sequence selected from the group consisting of sequences TATGCTGG and TATGCTGGA as disclosed in Table 3B. In some embodiments, the PEgRNA comprises an editing template comprising a sequence selected from the group consisting of SEQ Identifiers: C11340-C11370 of Table 3C. In some embodiments, the PEgRNA comprises an editing template comprising a sequence selected from the group consisting of SEQ ID NOs:15366-15396 of Table 3C. In some embodiments, the prime editing composition further comprises a ngRNA that comprises a spacer comprising a sequence selected from the group consisting of SEQ Identifiers: D1355-D1360, and D1385-D1390 of Table 3D. In some embodiments, the prime editing composition further comprises a ngRNA that comprises a spacer comprising a sequence selected from the group consisting of SEQ ID NOs. 20988-20993, and 21018-21023 of Table 3D.

In some embodiments, the PEgRNA comprises a spacer comprising a sequence selected from the group consisting of SEQ Identifiers: A595-A596 of Table 3A. In some embodiments, the PEgRNA comprises a spacer comprising a sequence as set forth in SEQ ID NO. 943-944 of Table 3A. In some embodiments, the PEgRNA comprises a PBS comprising a sequence selected from the group consisting of SEQ Identifiers: B2376-B2384 of Table 3B. In some embodiments, the PEgRNA comprises a PBS comprising a sequence selected from the group consisting of SEQ ID NOs. 3061-3067 of Table 3B. In some embodiments, the PEgRNA comprises a PBS comprising a sequence selected from the group consisting of sequences ATATTCAT and ATATTCATG as disclosed in Table 3B. In some embodiments, the PEgRNA comprises an editing template comprising a sequence selected from the group consisting of SEQ Identifiers: C11371-Cl1374 of Table 3C. In some embodiments, the PEgRNA comprises an editing template comprising a sequence selected from the group consisting of SEQ ID NOs:15397-15400 of Table 3C. In some embodiments, the prime editing composition further comprises a ngRNA that comprises a spacer comprising a sequence selected from the group consisting of SEQ Identifiers: D1355, and D1391-D1399 of Table 3D. In some embodiments, the prime editing composition further comprises a ngRNA that comprises a spacer comprising a sequence selected from the group consisting of SEQ ID NOs. 20988, and 21024-21032 of Table 3D.

In some embodiments, the PEgRNA comprises a spacer comprising the of SEQ Identifier: A597 of Table 3A. In some embodiments, the PEgRNA comprises a spacer comprising a sequence as set forth in SEQ ID NO. 945 of Table 3A. In some embodiments, the PEgRNA comprises a PBS comprising a sequence selected from the group consisting of SEQ Identifiers: B2385-B2393 of Table 3B. In some embodiments, the PEgRNA comprises a PBS comprising a sequence selected from the group consisting of SEQ ID NOs. 3068-3074 of Table 3B. In some embodiments, the PEgRNA comprises a PBS comprising a sequence selected from the group consisting of sequences GATATTCA and GATATTCAT as disclosed in Table 3B. In some embodiments, the PEgRNA comprises an editing template comprising a sequence selected from the group consisting of SEQ Identifiers: C11375-C11377 of Table 3C. In some embodiments, the PEgRNA comprises an editing template comprising a sequence selected from the group consisting of SEQ ID NOs:15401-15403 of Table 3C. In some embodiments, the prime editing composition further comprises a ngRNA that comprises a spacer comprising a sequence selected from the group consisting of SEQ Identifiers: D1367-D1380 of Table 3D. In some embodiments, the prime editing composition further comprises a ngRNA that comprises a spacer comprising a sequence selected from the group consisting of SEQ ID NOs. 21000-21013 of Table 3D.

In some embodiments, the PEgRNA comprises a spacer comprising a sequence selected from the group consisting of SEQ Identifier: A598-A599 of Table 3A. In some embodiments, the PEgRNA comprises a spacer comprising a sequence as set forth in SEQ ID NOs. 946-947 of Table 3A. In some embodiments, the PEgRNA comprises a PBS comprising a sequence selected from the group consisting of SEQ Identifiers: B2394-B2402 of Table 3B. In some embodiments, the PEgRNA comprises a PBS comprising a sequence selected from the group consisting of SEQ ID NOs. 3075-3081 of Table 3B. In some embodiments, the PEgRNA comprises a PBS comprising a sequence selected from the group consisting of sequences TGTACTCA and TGTACTCAT as disclosed in Table 3B. In some embodiments, the PEgRNA comprises an editing template comprising a sequence selected from the group consisting of SEQ Identifiers: C11378-C11401 of Table 3C. In some embodiments, the PEgRNA comprises an editing template comprising a sequence selected from the group consisting of SEQ ID NOs:15404-15427 of Table 3C. In some embodiments, the prime editing composition further comprises a ngRNA that comprises a spacer comprising a sequence selected from the group consisting of SEQ Identifiers: D1355, and D1391-D1399 of Table 3D. In some embodiments, the prime editing composition further comprises a ngRNA that comprises a spacer comprising a sequence selected from the group consisting of SEQ ID NOs. 20988, and 21024-21032 of Table 3D.

In some embodiments, the PEgRNA comprises a spacer comprising the sequence of SEQ Identifier: A600 of Table 3A. In some embodiments, the PEgRNA comprises a spacer comprising a sequence as set forth in SEQ ID NO. 948 of Table 3A. In some embodiments, the PEgRNA comprises a PBS comprising a sequence selected from the group consisting of SEQ Identifiers: B2403-B2411 of Table 3B. In some embodiments, the PEgRNA comprises a PBS comprising a sequence selected from the group consisting of SEQ ID NOs. 3082-3088 of Table 3B. In some embodiments, the PEgRNA comprises a PBS comprising a sequence selected from the group consisting of sequences ATGTACTC and ATGTACTCA as disclosed in Table 3B. In some embodiments, the PEgRNA comprises an editing template comprising a sequence selected from the group consisting of SEQ Identifiers: C11402-C11414 of Table 3C. In some embodiments, the PEgRNA comprises an editing template comprising a sequence selected from the group consisting of SEQ ID NOs:15428-15440 of Table 3C. In some embodiments, the prime editing composition further comprises a ngRNA that comprises a spacer comprising a sequence selected from the group consisting of SEQ Identifiers: D1367-D1380and D1383 of Table 3D. In some embodiments, the prime editing composition further comprises a ngRNA that comprises a spacer comprising a sequence selected from the group consisting of SEQ ID NOs. 21000-21013, and 21016 of Table 3D.

In some embodiments, the PEgRNA comprises a spacer comprising the sequence of SEQ Identifier: A601 of Table 3A. In some embodiments, the PEgRNA comprises a spacer comprising a sequence as set forth in SEQ ID NO. 949 of Table 3A. In some embodiments, the PEgRNA comprises a PBS comprising a sequence selected from the group consisting of SEQ Identifiers: B2412-B2420 of Table 3B. In some embodiments, the PEgRNA comprises a PBS comprising a sequence selected from the group consisting of SEQ ID NOs. 3089-3095 of Table 3B. In some embodiments, the PEgRNA comprises a PBS comprising a sequence selected from the group consisting of sequences GGATAGCT and GGATAGCTT as disclosed in Table 3B. In some embodiments, the PEgRNA comprises an editing template comprising a sequence selected from the group consisting of SEQ Identifiers: C11415-C11445 of Table 3C. In some embodiments, the PEgRNA comprises an editing template comprising a sequence selected from the group consisting of SEQ ID NOs:15441-15471 of Table 3C. In some embodiments, the prime editing composition further comprises a ngRNA that comprises a spacer comprising a sequence selected from the group consisting of SEQ Identifiers: D1315-D1329. and D1400-D1403 of Table 3D. In some embodiments, the prime editing composition further comprises a ngRNA that comprises a spacer comprising a sequence selected from the group consisting of SEQ ID NOs. 20948-20962, and 21033-21036 of Table 3D.

In some embodiments, the PEgRNA comprises a spacer comprising the of SEQ Identifier: A602 of Table 3A. In some embodiments, the PEgRNA comprises a spacer comprising a sequence as set forth in SEQ ID NO. 950 of Table 3A. In some embodiments, the PEgRNA comprises a PBS comprising a sequence selected from the group consisting of SEQ Identifiers: B2421-B2429 of Table 3B. In some embodiments, the PEgRNA comprises a PBS comprising a sequence selected from the group consisting of SEQ ID NOs. 3096-3102 of Table 3B. In some embodiments, the PEgRNA comprises a PBS comprising a sequence selected from the group consisting of sequences ATGCTGGA and ATGCTGGAG as disclosed in Table 3B. In some embodiments, the PEgRNA comprises an editing template comprising a sequence selected from the group consisting of SEQ Identifiers: C11446-C11476 of Table 3C. In some embodiments, the PEgRNA comprises an editing template comprising a sequence selected from the group consisting of SEQ ID NOs:15472-15502 of Table 3C. In some embodiments, the prime editing composition further comprises a ngRNA that comprises a spacer comprising a sequence selected from the group consisting of SEQ Identifiers: D1370-D1380, D1384, D1386, D1404, D1405, D1406, and D1407 of Table 3D. In some embodiments, the prime editing composition further comprises a ngRNA that comprises a spacer comprising a sequence selected from the group consisting of SEQ ID NOs. 21003-21013, 21017, 21019, 21037, 21038, 21039, and 21040 of Table 3D.

In some embodiments, the PEgRNA comprises a spacer comprising a sequence selected from the group consisting of SEQ Identifiers: A603-A604 of Table 3A. In some embodiments, the PEgRNA comprises a spacer comprising a sequence as set forth in SEQ ID NOs. 951-952 of Table 3A. In some embodiments, the PEgRNA comprises a PBS comprising a sequence selected from the group consisting of SEQ Identifiers: B2430-B2438 of Table 3B. In some embodiments, the PEgRNA comprises a PBS comprising a sequence selected from the group consisting of SEQ ID NOs. 3103-3109 of Table 3B. In some embodiments, the PEgRNA comprises a PBS comprising a sequence selected from the group consisting of sequences ATCTTTTT and ATCTTTTTA as disclosed in Table 3B. In some embodiments, the PEgRNA comprises an editing template comprising a sequence selected from the group consisting of SEQ Identifiers: C11477-C11502 of Table 3C. In some embodiments, the PEgRNA comprises an editing template comprising a sequence selected from the group consisting of SEQ ID NOs:15503-15528 of Table 3C. In some embodiments, the prime editing composition further comprises a ngRNA that comprises a spacer comprising a sequence selected from the group consisting of SEQ Identifiers: D1335-D1354, D1408, and D1409 of Table 3D. In some embodiments, the prime editing composition further comprises a ngRNA that comprises a spacer comprising a sequence selected from the group consisting of SEQ ID NOs. 20968-20987, 21041, and 21042 of Table 3D.

In some embodiments, the PEgRNA comprises a spacer comprising the sequence of SEQ Identifier: A605 of Table 3A. In some embodiments, the PEgRNA comprises a spacer comprising a sequence as set forth in SEQ ID NO. 953 of Table 3A. In some embodiments, the PEgRNA comprises a PBS comprising a sequence selected from the group consisting of SEQ Identifiers: B2439-B2447 of Table 3B. In some embodiments, the PEgRNA comprises a PBS comprising a sequence selected from the group consisting of SEQ ID NOs. 3110-3116 of Table 3B. In some embodiments, the PEgRNA comprises a PBS comprising a sequence selected from the group consisting of sequences ATGTGGAT and ATGTGGATA as disclosed in Table 3B. In some embodiments, the PEgRNA comprises an editing template comprising a sequence selected from the group consisting of SEQ Identifiers: C11503-Cl1533 of Table 3C. In some embodiments, the PEgRNA comprises an editing template comprising a sequence selected from the group consisting of SEQ ID NOs:15529-15559 of Table 3C. In some embodiments, the prime editing composition further comprises a ngRNA that comprises a spacer comprising a sequence selected from the group consisting of SEQ Identifiers: D1315-D1329, D1400-D1403, and D1410 of Table 3D. In some embodiments, the prime editing composition further comprises a ngRNA that comprises a spacer comprising a sequence selected from the group consisting of SEQ ID NOs. 20948-20962, 21033-21036, and 21043 of Table 3D.

In some embodiments, the PEgRNA comprises a spacer comprising a sequence selected from the group consisting of SEQ Identifiers: A606-A609 of Table 3A. In some embodiments, the PEgRNA comprises a spacer comprising a sequence as set forth in SEQ ID NOs. 954-957 of Table 3A. In some embodiments, the PEgRNA comprises a PBS comprising a sequence selected from the group consisting of SEQ Identifiers: B2448-B2456 of Table 3B. In some embodiments, the PEgRNA comprises a PBS comprising a sequence selected from the group consisting of SEQ ID NOs. 3117-3123 of Table 3B. In some embodiments, the PEgRNA comprises a PBS comprising a sequence selected from the group consisting of sequences CAATGTAC and CAATGTACT as disclosed in Table 3B. In some embodiments, the PEgRNA comprises an editing template comprising a sequence selected from the group consisting of SEQ Identifiers: C11534-C11548 of Table 3C. In some embodiments, the PEgRNA comprises an editing template comprising a sequence selected from the group consisting of SEQ ID NOs:15560-15574 of Table 3C. In some embodiments, the prime editing composition further comprises a ngRNA that comprises a spacer comprising a sequence selected from the group consisting of SEQ Identifiers: D1355-D1360, Dand 1367-D1383 of Table 3D. In some embodiments, the prime editing composition further comprises a ngRNA that comprises a spacer comprising a sequence selected from the group consisting of SEQ ID NOs. 20988-20993, and 21000-21016 of Table 3D.

In some embodiments, the PEgRNA comprises a spacer comprising a sequence selected from the group consisting of SEQ Identifiers: A610-A611 of Table 3A. In some embodiments, the PEgRNA comprises a spacer comprising a sequence as set forth in SEQ ID NOs. 958-959 of Table 3A. In some embodiments, the PEgRNA comprises a PBS comprising a sequence selected from the group consisting of SEQ Identifiers: B2457-B2465 of Table 3B. In some embodiments, the PEgRNA comprises a PBS comprising a sequence selected from the group consisting of SEQ ID NOs. 3124-3130 of Table 3B. In some embodiments, the PEgRNA comprises a PBS comprising a sequence selected from the group consisting of sequences TTTACAGC and TTTACAGCC as disclosed in Table 3B. In some embodiments, the PEgRNA comprises an editing template comprising a sequence selected from the group consisting of SEQ Identifiers: C11549-C11606 of Table 3C. In some embodiments, the PEgRNA comprises an editing template comprising a sequence selected from the group consisting of SEQ ID NOs:15575-15632 of Table 3C. In some embodiments, the prime editing composition further comprises a ngRNA that comprises a spacer comprising a sequence selected from the group consisting of SEQ Identifiers: D1355, D1391, D1392, D1394-D1397, D1399, and D1411-D1414 of Table 3D. In some embodiments, the prime editing composition further comprises a ngRNA that comprises a spacer comprising a sequence selected from the group consisting of SEQ ID NOs. 20988, 21024, 21025, 21027-21030, 21032, and 21044-21047 of Table 3D.

In some embodiments, the PEgRNA comprises a spacer comprising the sequence of SEQ Identifier: A612 of Table 3A. In some embodiments, the PEgRNA comprises a spacer comprising a sequence as set forth in SEQ ID NO. 960 of Table 3A. In some embodiments, the PEgRNA comprises a PBS comprising a sequence selected from the group consisting of SEQ Identifiers: B2466-B2474 of Table 3B. In some embodiments, the PEgRNA comprises a PBS comprising a sequence selected from the group consisting of SEQ ID NOs. 3131-3137 of Table 3B. In some embodiments, the PEgRNA comprises a PBS comprising a sequence selected from the group consisting of sequences GTTTACAG and GTTTACAGC as disclosed in Table 3B. In some embodiments, the PEgRNA comprises an editing template comprising a sequence selected from the group consisting of SEQ Identifiers: C11607-C11636 of Table 3C. In some embodiments, the PEgRNA comprises an editing template comprising a sequence selected from the group consisting of SEQ ID NOs:15633-15662 of Table 3C. In some embodiments, the prime editing composition further comprises a ngRNA that comprises a spacer comprising a sequence selected from the group consisting of SEQ Identifiers: D1370-D1380, D1383, D1384, D1404, and D1405 of Table 3D. In some embodiments, the prime editing composition further comprises a ngRNA that comprises a spacer comprising a sequence selected from the group consisting of SEQ ID NOs. 21003-21013, 21016, 21017, 21037, and 21038 of Table 3D.

In some embodiments, the PEgRNA comprises a spacer comprising a sequence selected from the group consisting of SEQ Identifiers: A613-A614 of Table 3A. In some embodiments, the PEgRNA comprises a spacer comprising a sequence as set forth in SEQ ID NOs. 961-962 of Table 3A. In some embodiments, the PEgRNA comprises a PBS comprising a sequence selected from the group consisting of SEQ Identifiers: B2475-B2483 of Table 3B. In some embodiments, the PEgRNA comprises a PBS comprising a sequence selected from the group consisting of SEQ ID NOs. 3138-3144 of Table 3B. In some embodiments, the PEgRNA comprises a PBS comprising a sequence selected from the group consisting of sequences TTTTAACT and TTTTAACTT as disclosed in Table 3B. In some embodiments, the PEgRNA comprises an editing template comprising a sequence selected from the group consisting of SEQ Identifiers: C11637-C11645 of Table 3C. In some embodiments, the PEgRNA comprises an editing template comprising a sequence selected from the group consisting of SEQ ID NOs:15663-15671 of Table 3C. In some embodiments, the prime editing composition further comprises a ngRNA that comprises a spacer comprising a sequence selected from the group consisting of SEQ Identifiers: D1305-D1334 of Table 3D. In some embodiments, the prime editing composition further comprises a ngRNA that comprises a spacer comprising a sequence selected from the group consisting of SEQ ID NOs. 20938-20967 of Table 3D.

In some embodiments, the PEgRNA comprises a spacer comprising a sequence selected from the group consisting of SEQ Identifiers: A615-A616 of Table 3A. In some embodiments, the PEgRNA comprises a spacer comprising a sequence as set forth in SEQ ID NOs. 963-964 of Table 3A. In some embodiments, the PEgRNA comprises a PBS comprising a sequence selected from the group consisting of SEQ Identifiers: B2484-B2492 of Table 3B. In some embodiments, the PEgRNA comprises a PBS comprising a sequence selected from the group consisting of SEQ ID NOs. 3145-3151 of Table 3B. In some embodiments, the PEgRNA comprises a PBS comprising a sequence selected from the group consisting of sequences TAACTTTT and TAACTTTTA as disclosed in Table 3B. In some embodiments, the PEgRNA comprises an editing template comprising a sequence selected from the group consisting of SEQ Identifiers: C11646-C11651 of Table 3C. In some embodiments, the PEgRNA comprises an editing template comprising a sequence selected from the group consisting of SEQ ID NOs:15672-15677 of Table 3C. In some embodiments, the prime editing composition further comprises a ngRNA that comprises a spacer comprising a sequence selected from the group consisting of SEQ Identifiers: D1305-D1334 of Table 3D. In some embodiments, the prime editing composition further comprises a ngRNA that comprises a spacer comprising a sequence selected from the group consisting of SEQ ID NOs. 20938-20967 of Table 3D.

In some embodiments, the PEgRNA comprises a spacer comprising a sequence selected from the group consisting of SEQ Identifiers: A617-A618 of Table 3A. In some embodiments, the PEgRNA comprises a spacer comprising a sequence as set forth in SEQ Identifiers. 965-966 of Table 3A. In some embodiments, the PEgRNA comprises a PBS comprising a sequence selected from the group consisting of SEQ Identifiers: B2493-B2501 of Table 3B. In some embodiments, the PEgRNA comprises a PBS comprising a sequence selected from the group consisting of SEQ ID NOs. 3152-3158 of Table 3B. In some embodiments, the PEgRNA comprises a PBS comprising a sequence selected from the group consisting of sequences AACTTTTA and AACTTTTAT as disclosed in Table 3B. In some embodiments, the PEgRNA comprises an editing template comprising a sequence selected from the group consisting of SEQ Identifiers: C11652-C11661 of Table 3C. In some embodiments, the PEgRNA comprises an editing template comprising a sequence selected from the group consisting of SEQ ID NOs:15678-15687 of Table 3C. In some embodiments, the prime editing composition further comprises a ngRNA that comprises a spacer comprising a sequence selected from the group consisting of SEQ Identifiers: D1335-D1354, D1408, and D1409 of Table 3D. In some embodiments, the prime editing composition further comprises a ngRNA that comprises a spacer comprising a sequence selected from the group consisting of SEQ ID NOs. 20968-20987, 21041, and 21042 of Table 3D.

In some embodiments, the editing target sequence is in exon 22 of the CFTR gene.

In some embodiments, the editing target sequence or the complementary sequence on the CFTR gene thereof comprises a mutation that encodes an amino acid substitution R1158*, R1162*, Ri 162L, 51161R, R1162L, and/or K1165T relative to a wild type CFTR polypeptide set forth in SEQ ID NO: 19, and wherein the editing template encodes an intended nucleotide edit that corrects the mutation. In some embodiments, the editing target sequence or the complementary sequence on the CFTR gene thereof comprises a C→T mutation (c.3472C>T) at position 117,627,525, a C→G mutation (c.3483C>G) at position 117,627,536, a G→T mutation (c.3485G>T) at position 117,627,538, a deletion of nucleotides AG (c.3486_3487delAG) at position 117,627,539, a duplication of a nucleotide T (c.3492dupT) at position 117,627,545, a A→C mutation (c.3494A>C) at position 117,627,547, a deletion of G (c.3495delG) at position 117,627,548, a C→A mutation (c.3469-3C>A) at position 117,627,519, an A→G mutation (c.3469-2A>G) at position 117,627,520, and/or a deletion of nucleotide T (c.3477delT) at position 117,627,530 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation.

In some embodiments, the editing template comprises a wild type CFTR gene sequence. In some embodiments, the editing template comprises one or more intended nucleotide edits compared to the target CFTR gene sequence in exon 22 of the target CFTR gene. Accordingly, in some embodiments, the newly synthesized single stranded DNA comprises one or more intended nucleotide edits compared to the target CFTR gene sequence. In some embodiments, the newly synthesized single stranded DNA encoded by the editing template is capable of correcting the c.3472C>T mutation, the c.3483C>G mutation, the c.3485G>T mutation, the c.3486_3487delAG mutation, the c.3492dupT mutation, the c.3494A>C mutation, the c.3495delG mutation the c.3469-3C>A mutation, the c.3469-2A>G mutation, and/or the c.3477delT mutation in the target CFTR gene. In some embodiments, the newly synthesized single stranded DNA encoded by the editing template is capable of correcting all of the c.3472C>T mutation, the c.3469-3C>A mutation, the c.3469-2A>G mutation, and/or the c.3477delT mutation in the target CFTR gene. In some embodiments, two or more of the c.3472C>T mutation, the c.3469-3C>A mutation, the c.3469-2A>G mutation, and the c.3477delT mutation are located on the same CFTR gene. In some embodiments, the newly synthesized single stranded DNA encoded by the editing template is capable of correcting the c.3472C>T mutation, the c.3483C>G mutation, the c.3485G>T mutation, the c.3486_3487delAG mutation, the c.3492dupT mutation, the c.3494A>C mutation, the c.3495delG mutation, the c.3469-3C>A mutation, the c.3469-2A>G mutation, and the c.3477delT mutation that are located on different CFTR genes. For example, in some embodiments, a subject may comprise two copies of CFTR genes, each comprising a different mutation selected from the group consisting of the c.3472C>T mutation, the c.3469-3C>A mutation, the c.3469-2A>G mutation, the c.3483C>G mutation, the c.3485G>T mutation, the c.3486_3487delAG mutation, the c.3492dupT mutation, the c.3494A>C mutation, the c.3495delG mutation, and the c.3477delT mutation, all of which may be corrected by an editing template of a single PEgRNA. In some embodiments, the editing template is capable of correcting one or more, or all of the c.3472C>T mutation, the c.3469-3C>A mutation, the c.3469-2A>G mutation, the c.3483C>G mutation, the c.3485G>T mutation, the c.3486_3487delAG mutation, the c.3492dupT mutation, the c.3494A>C mutation, the c.3495delG mutation, and the c.3477delT mutation that are in different subjects, wherein each subject may have one or more of the mutations.

In some embodiments, the PEgRNA comprises a spacer comprising a sequence selected from the group consisting of SEQ Identifiers: A619-A620 of Table 3A. In some embodiments, the PEgRNA comprises a spacer comprising a sequence as set forth in SEQ ID NOs. 967-968 of Table 3A. In some embodiments, the PEgRNA comprises a PBS comprising a sequence selected from the group consisting of SEQ Identifiers: B2502-B2510 of Table 3B. In some embodiments, the PEgRNA comprises a PBS comprising a sequence selected from the group consisting of SEQ ID NOs. 3159-3165 of Table 3B. In some embodiments, the PEgRNA comprises a PBS comprising a sequence selected from the group consisting of sequences GCCGAGTC and GCCGAGTCT as disclosed in Table 3B. In some embodiments, the PEgRNA comprises an editing template comprising a sequence selected from the group consisting of SEQ Identifiers: C11662-C11721 of Table 3C. In some embodiments, the PEgRNA comprises an editing template comprising a sequence selected from the group consisting of SEQ ID NOs: 15688-15747 of Table 3C. In some embodiments, the prime editing composition further comprises a ngRNA that comprises a spacer comprising a sequence selected from the group consisting of SEQ Identifiers: D1415-D1434 of Table 3D. In some embodiments, the prime editing composition further comprises a ngRNA that comprises a spacer comprising a sequence selected from the group consisting of SEQ ID NOs. 21048-21067 of Table 3D.

In some embodiments, the PEgRNA comprises a spacer comprising a sequence selected from the group consisting of SEQ Identifiers: A621-A622 of Table 3A. In some embodiments, the PEgRNA comprises a spacer comprising a sequence as set forth in SEQ ID NOs. 969-970 of Table 3A. In some embodiments, the PEgRNA comprises a PBS comprising a sequence selected from the group consisting of SEQ Identifiers: B2511-B2519 of Table 3B. In some embodiments, the PEgRNA comprises a PBS comprising a sequence selected from the group consisting of SEQ ID NOs. 3166-3172 of Table 3B. In some embodiments, the PEgRNA comprises a PBS comprising a sequence selected from the group consisting of sequences TAAAAATA and TAAAAATAA as disclosed in Table 3B. In some embodiments, the PEgRNA comprises an editing template comprising a sequence selected from the group consisting of SEQ Identifiers: C11722-C11781 of Table 3C. In some embodiments, the PEgRNA comprises an editing template comprising a sequence selected from the group consisting of SEQ ID NOs:15748-15807 of Table 3C. In some embodiments, the prime editing composition further comprises a ngRNA that comprises a spacer comprising a sequence selected from the group consisting of SEQ Identifiers: D1435-D1440 of Table 3D. In some embodiments, the prime editing composition further comprises a ngRNA that comprises a spacer comprising a sequence selected from the group consisting of SEQ ID NOs. 21068-21073 of Table 3D.

In some embodiments, the PEgRNA comprises a spacer comprising a sequence selected from the group consisting of SEQ Identifiers: A623-A624 of Table 3A. In some embodiments, the PEgRNA comprises a spacer comprising a sequence as set forth in SEQ ID NOs. 971-972 of Table 3A. In some embodiments, the PEgRNA comprises a PBS comprising a sequence selected from the group consisting of SEQ Identifiers: B2520-B2528 of Table 3B. In some embodiments, the PEgRNA comprises a PBS comprising a sequence selected from the group consisting of SEQ ID NOs. 3173-3179 of Table 3B. In some embodiments, the PEgRNA comprises a PBS comprising a sequence selected from the group consisting of sequences AGCCGAGT and AGCCGAGTC as disclosed in Table 3B. In some embodiments, the PEgRNA comprises an editing template comprising a sequence selected from the group consisting of SEQ Identifiers: C11782-C11812 of Table 3C. In some embodiments, the PEgRNA comprises an editing template comprising a sequence selected from the group consisting of SEQ ID NOs:15808-15838 of Table 3C. In some embodiments, the prime editing composition further comprises a ngRNA that comprises a spacer comprising a sequence selected from the group consisting of SEQ Identifiers: D1441-D1472 of Table 3D. In some embodiments, the prime editing composition further comprises a ngRNA that comprises a spacer comprising a sequence selected from the group consisting of SEQ ID NOs. 21074-21105 of Table 3D.

In some embodiments, the PEgRNA comprises a spacer comprising the sequence of SEQ Identifier: A625 of Table 3A. In some embodiments, the PEgRNA comprises a spacer comprising a sequence as set forth in SEQ ID NO. 973 of Table 3A. In some embodiments, the PEgRNA comprises a PBS comprising a sequence selected from the group consisting of SEQ Identifiers: B2529-B2537 of Table 3B. In some embodiments, the PEgRNA comprises a PBS comprising a sequence selected from the group consisting of SEQ ID NOs. 3180-3186 of Table 3B. In some embodiments, the PEgRNA comprises a PBS comprising a sequence selected from the group consisting of sequences CAACAGAA and CAACAGAAG as disclosed in Table 3B. In some embodiments, the PEgRNA comprises an editing template comprising a sequence selected from the group consisting of SEQ Identifiers: C11813-C11815 of Table 3C. In some embodiments, the PEgRNA comprises an editing template comprising a sequence selected from the group consisting of SEQ ID NOs:15839-15841 of Table 3C. In some embodiments, the prime editing composition further comprises a ngRNA that comprises a spacer comprising a sequence selected from the group consisting of SEQ Identifiers: D1445-D1461, and D1473-D1478 of Table 3D. In some embodiments, the prime editing composition further comprises a ngRNA that comprises a spacer comprising a sequence selected from the group consisting of SEQ ID NOs. 21078-21094, and 21106-21111 of Table 3D.

In some embodiments, the PEgRNA comprises a spacer comprising a sequence selected from the group consisting of SEQ Identifiers: A626-A628 of Table 3A. In some embodiments, the PEgRNA comprises a spacer comprising a sequence as set forth in SEQ ID NOs. 974-976 of Table 3A. In some embodiments, the PEgRNA comprises a PBS comprising a sequence selected from the group consisting of SEQ Identifiers: B2538-B2546 of Table 3B. In some embodiments, the PEgRNA comprises a PBS comprising a sequence selected from the group consisting of SEQ ID NOs. 3187-3193 of Table 3B. In some embodiments, the PEgRNA comprises a PBS comprising a sequence selected from the group consisting of sequences TTTAAGTT and TTTAAGTTC as disclosed in Table 3B. In some embodiments, the PEgRNA comprises an editing template comprising a sequence selected from the group consisting of SEQ Identifiers: C11816-C11837 of Table 3C. In some embodiments, the PEgRNA comprises an editing template comprising a sequence selected from the group consisting of SEQ ID NOs:15842-15863 of Table 3C. In some embodiments, the prime editing composition further comprises a ngRNA that comprises a spacer comprising a sequence selected from the group consisting of SEQ Identifiers: D1427, and D1479-D1486 of Table 3D. In some embodiments, the prime editing composition further comprises a ngRNA that comprises a spacer comprising a sequence selected from the group consisting of SEQ ID NOs. 21060, and 21112-21119 of Table 3D.

In some embodiments, the PEgRNA comprises a spacer comprising a sequence selected from the group consisting of SEQ Identifiers: A629-A630 of Table 3A. In some embodiments, the PEgRNA comprises a spacer comprising a sequence as set forth in SEQ ID NOs. 977-978 of Table 3A. In some embodiments, the PEgRNA comprises a PBS comprising a sequence selected from the group consisting of SEQ Identifiers: B2547-B2555 of Table 3B. In some embodiments, the PEgRNA comprises a PBS comprising a sequence selected from the group consisting of SEQ ID NOs. 3194-3200 of Table 3B. In some embodiments, the PEgRNA comprises a PBS comprising a sequence selected from the group consisting of sequences ATAAAAAT and ATAAAAATA as disclosed in Table 3B. In some embodiments, the PEgRNA comprises an editing template comprising a sequence selected from the group consisting of SEQ Identifiers: C11838-C11868 of Table 3C. In some embodiments, the PEgRNA comprises an editing template comprising a sequence selected from the group consisting of SEQ ID NOs:15864-15894 of Table 3C. In some embodiments, the prime editing composition further comprises a ngRNA that comprises a spacer comprising a sequence selected from the group consisting of SEQ Identifiers: D1487-D1507 of Table 3D. In some embodiments, the prime editing composition further comprises a ngRNA that comprises a spacer comprising a sequence selected from the group consisting of SEQ ID NOs. 21120-21140 of Table 3D.

In some embodiments, the PEgRNA comprises a spacer comprising the sequence of SEQ Identifier: A631 of Table 3A. In some embodiments, the PEgRNA comprises a spacer comprising a sequence as set forth in SEQ ID NO. 979 of Table 3A. In some embodiments, the PEgRNA comprises a PBS comprising a sequence selected from the group consisting of SEQ Identifiers: B2556-B2564 of Table 3B. In some embodiments, the PEgRNA comprises a PBS comprising a sequence selected from the group consisting of SEQ ID NOs. 3201-3207 of Table 3B. In some embodiments, the PEgRNA comprises a PBS comprising a sequence selected from the group consisting of sequences TTTTTGTT and TTTTTGTTT as disclosed in Table 3B. In some embodiments, the PEgRNA comprises an editing template comprising a sequence selected from the group consisting of SEQ Identifiers: C11869-C11884 of Table 3C. In some embodiments, the PEgRNA comprises an editing template comprising a sequence selected from the group consisting of SEQ ID NOs:15895-15910 of Table 3C. In some embodiments, the prime editing composition further comprises a ngRNA that comprises a spacer comprising a sequence selected from the group consisting of SEQ Identifiers: D1487-D1504 and D1508 of Table 3D. In some embodiments, the prime editing composition further comprises a ngRNA that comprises a spacer comprising a sequence selected from the group consisting of SEQ ID NOs. 21120-21137, and 21141 of Table 3D.

In some embodiments, the PEgRNA comprises a spacer comprising a sequence selected from the group consisting of SEQ Identifiers: A632-A633 of Table 3A. In some embodiments, the PEgRNA comprises a spacer comprising a sequence as set forth in SEQ ID NOs. 980-981 of Table 3A. In some embodiments, the PEgRNA comprises a PBS comprising a sequence selected from the group consisting of SEQ Identifiers: B2565-B2573 of Table 3B. In some embodiments, the PEgRNA comprises a PBS comprising a sequence selected from the group consisting of SEQ ID NOs. 3208-3214 of Table 3B. In some embodiments, the PEgRNA comprises a PBS comprising a sequence selected from the group consisting of sequences ACATGCCA and ACATGCCAA as disclosed in Table 3B. In some embodiments, the PEgRNA comprises an editing template comprising a sequence selected from the group consisting of SEQ Identifiers: C11885-C11893 of Table 3C. In some embodiments, the PEgRNA comprises an editing template comprising a sequence selected from the group consisting of SEQ ID NOs:15911-15919 of Table 3C. In some embodiments, the prime editing composition further comprises a ngRNA that comprises a spacer comprising a sequence selected from the group consisting of SEQ Identifiers: D1445-D1461, and D1463-D1478 of Table 3D. In some embodiments, the prime editing composition further comprises a ngRNA that comprises a spacer comprising a sequence selected from the group consisting of SEQ ID NOs. 21078-21094, and 21096-21111 of Table 3D.

In some embodiments, the PEgRNA comprises a spacer comprising the sequence of SEQ Identifier: A634 of Table 3A. In some embodiments, the PEgRNA comprises a spacer comprising a sequence as set forth in SEQ ID NO. 982 of Table 3A. In some embodiments, the PEgRNA comprises a PBS comprising a sequence selected from the group consisting of SEQ Identifiers: B2574-B2582 of Table 3B. In some embodiments, the PEgRNA comprises a PBS comprising a sequence selected from the group consisting of SEQ ID NOs. 3215-3221 of Table 3B. In some embodiments, the PEgRNA comprises a PBS comprising a sequence selected from the group consisting of sequences TTGTTTTT and TTGTTTTTA as disclosed in Table 3B. In some embodiments, the PEgRNA comprises an editing template comprising a sequence selected from the group consisting of SEQ Identifiers: C11894-C11906 of Table 3C. In some embodiments, the PEgRNA comprises an editing template comprising a sequence selected from the group consisting of SEQ ID NOs:15920-15932 of Table 3C. In some embodiments, the prime editing composition further comprises a ngRNA that comprises a spacer comprising a sequence selected from the group consisting of SEQ Identifiers: D1487-D1503 D1508, D1509, and D1510 of Table 3D. In some embodiments, the prime editing composition further comprises a ngRNA that comprises a spacer comprising a sequence selected from the group consisting of SEQ ID NOs. 21120-21136, 21141, 21142, and 21143 of Table 3D.

In some embodiments, the PEgRNA comprises a spacer comprising a sequence selected from the group consisting of SEQ Identifiers: A635-A636 of Table 3A. In some embodiments, the PEgRNA comprises a spacer comprising a sequence as set forth in SEQ ID NOs. 983-984 of Table 3A. In some embodiments, the PEgRNA comprises a PBS comprising a sequence selected from the group consisting of SEQ Identifiers: B2583-B2591 of Table 3B. In some embodiments, the PEgRNA comprises a PBS comprising a sequence selected from the group consisting of SEQ ID NOs. 3222-3228 of Table 3B. In some embodiments, the PEgRNA comprises a PBS comprising a sequence selected from the group consisting of sequences AGTTCATT and AGTTCATTG as disclosed in Table 3B. In some embodiments, the PEgRNA comprises an editing template comprising a sequence selected from the group consisting of SEQ Identifiers: C11907-C11924 of Table 3C. In some embodiments, the PEgRNA comprises an editing template comprising a sequence selected from the group consisting of SEQ ID NOs:15933-15950 of Table 3C. In some embodiments, the prime editing composition further comprises a ngRNA that comprises a spacer comprising a sequence selected from the group consisting of SEQ Identifiers: D1443-D1474 of Table 3D. In some embodiments, the prime editing composition further comprises a ngRNA that comprises a spacer comprising a sequence selected from the group consisting of SEQ ID NOs. 21076-21107 of Table 3D.

In some embodiments, the PEgRNA comprises a spacer comprising a sequence selected from the group consisting of SEQ Identifiers: A637-A638 of Table 3A. In some embodiments, the PEgRNA comprises a spacer comprising a sequence as set forth in SEQ ID NOs. 985-986 of Table 3A. In some embodiments, the PEgRNA comprises a PBS comprising a sequence selected from the group consisting of SEQ Identifiers: B2592-B2600 of Table 3B. In some embodiments, the PEgRNA comprises a PBS comprising a sequence selected from the group consisting of SEQ ID NOs. 3229-3235 of Table 3B. In some embodiments, the PEgRNA comprises a PBS comprising a sequence selected from the group consisting of sequences CTTTAAGT and CTTTAAGTT as disclosed in Table 3B. In some embodiments, the PEgRNA comprises an editing template comprising a sequence selected from the group consisting of SEQ Identifiers: C11925-C11970 of Table 3C. In some embodiments, the PEgRNA comprises an editing template comprising a sequence selected from the group consisting of SEQ ID NOs:15951-15996 of Table 3C. In some embodiments, the prime editing composition further comprises a ngRNA that comprises a spacer comprising a sequence selected from the group consisting of SEQ Identifiers: D1442-D1461, D1473, D1474, D1511, D1512, and D1513 of Table 3D. In some embodiments, the prime editing composition further comprises a ngRNA that comprises a spacer comprising a sequence selected from the group consisting of SEQ ID NOs. 21075-21094, 21106, 21107, 21144, 21145, and 21146 of Table 3D.

In some embodiments, the PEgRNA comprises a spacer comprising the sequence of SEQ Identifier: A639 of Table 3A. In some embodiments, the PEgRNA comprises a spacer comprising a sequence as set forth in SEQ ID NO. 987 of Table 3A. In some embodiments, the PEgRNA comprises a PBS comprising a sequence selected from the group consisting of SEQ Identifiers: B2601-B2609 of Table 3B. In some embodiments, the PEgRNA comprises a PBS comprising a sequence selected from the group consisting of SEQ ID NOs. 3236-3242 of Table 3B. In some embodiments, the PEgRNA comprises a PBS comprising a sequence selected from the group consisting of sequences TCTTTAAG and TCTTTAAGT as disclosed in Table 3B. In some embodiments, the PEgRNA comprises an editing template comprising a sequence selected from the group consisting of SEQ Identifiers: C11971-C11994 of Table 3C. In some embodiments, the PEgRNA comprises an editing template comprising a sequence selected from the group consisting of SEQ ID NOs:15997-16020 of Table 3C. In some embodiments, the prime editing composition further comprises a ngRNA that comprises a spacer comprising a sequence selected from the group consisting of SEQ Identifiers: D1442-D1461. D1473, and D1474 of Table 3D. In some embodiments, the prime editing composition further comprises a ngRNA that comprises a spacer comprising a sequence selected from the group consisting of SEQ ID NOs. 21075-21094, 21106, and 21107 of Table 3D.

In some embodiments, the PEgRNA comprises a spacer comprising a sequence selected from the group consisting of SEQ Identifiers: A640-A641 of Table 3A. In some embodiments, the PEgRNA comprises a spacer comprising a sequence as set forth in SEQ ID NOs. 988-989 of Table 3A. In some embodiments, the PEgRNA comprises a PBS comprising a sequence selected from the group consisting of SEQ Identifiers: B2610-B2618 of Table 3B. In some embodiments, the PEgRNA comprises a PBS comprising a sequence selected from the group consisting of SEQ ID NOs. 3243-3249 of Table 3B. In some embodiments, the PEgRNA comprises a PBS comprising a sequence selected from the group consisting of sequences CTGAAATA and CTGAAATAA as disclosed in Table 3B. In some embodiments, the PEgRNA comprises an editing template comprising a sequence selected from the group consisting of SEQ Identifiers: C11995-C12025 of Table 3C. In some embodiments, the PEgRNA comprises an editing template comprising a sequence selected from the group consisting of SEQ ID NOs:16021-16051 of Table 3C. In some embodiments, the prime editing composition further comprises a ngRNA that comprises a spacer comprising a sequence selected from the group consisting of SEQ Identifiers: D1489-D1507 of Table 3D. In some embodiments, the prime editing composition further comprises a ngRNA that comprises a spacer comprising a sequence selected from the group consisting of SEQ ID NOs. 21122-21140 of Table 3D.

In some embodiments, the PEgRNA comprises a spacer comprising the sequence of SEQ Identifier: A642 of Table 3A. In some embodiments, the PEgRNA comprises a spacer comprising a sequence as set forth in SEQ ID NO. 990 of Table 3A. In some embodiments, the PEgRNA comprises a PBS comprising a sequence selected from the group consisting of SEQ Identifiers: B2619-B2627 of Table 3B. In some embodiments, the PEgRNA comprises a PBS comprising a sequence selected from the group consisting of SEQ ID NOs. 3250-3256 of Table 3B. In some embodiments, the PEgRNA comprises a PBS comprising a sequence selected from the group consisting of sequences GAAATAAA and GAAATAAAA as disclosed in Table 3B. In some embodiments, the PEgRNA comprises an editing template comprising a sequence selected from the group consisting of SEQ Identifiers: C12026-C12056 of Table 3C. In some embodiments, the PEgRNA comprises an editing template comprising a sequence selected from the group consisting of SEQ ID NOs:16052-16082 of Table 3C. In some embodiments, the prime editing composition further comprises a ngRNA that comprises a spacer comprising a sequence selected from the group consisting of SEQ Identifiers: D1487-D1504 of Table 3D. In some embodiments, the prime editing composition further comprises a ngRNA that comprises a spacer comprising a sequence selected from the group consisting of SEQ ID NOs. 21120-21137 of Table 3D.

In some embodiments, the PEgRNA comprises a spacer comprising a sequence selected from the group consisting of SEQ Identifiers: A643-A644 of Table 3A. In some embodiments, the PEgRNA comprises a spacer comprising a sequence as set forth in SEQ ID NOs. 991-992 of Table 3A. In some embodiments, the PEgRNA comprises a PBS comprising a sequence selected from the group consisting of SEQ Identifiers: B2628-B2636 of Table 3B. In some embodiments, the PEgRNA comprises a PBS comprising a sequence selected from the group consisting of SEQ ID NOs. 3257-3263 of Table 3B. In some embodiments, the PEgRNA comprises a PBS comprising a sequence selected from the group consisting of sequences GTTCATTG and GTTCATTGA as disclosed in Table 3B. In some embodiments, the PEgRNA comprises an editing template comprising a sequence selected from the group consisting of SEQ Identifiers: C12057-C12090 of Table 3C. In some embodiments, the PEgRNA comprises an editing template comprising a sequence selected from the group consisting of SEQ ID NOs:16083-16116 of Table 3C. In some embodiments, the prime editing composition further comprises a ngRNA that comprises a spacer comprising a sequence selected from the group consisting of SEQ Identifiers: D1416-D1423, D1426-D1433, D1514, and D1515 of Table 3D. In some embodiments, the prime editing composition further comprises a ngRNA that comprises a spacer comprising a sequence selected from the group consisting of SEQ ID NOs. 21049-21056, 21059-21066, 21147, and 21148 of Table 3D.

In some embodiments, the PEgRNA comprises a spacer comprising a sequence selected from the group consisting of SEQ Identifiers: A621-A622 of Table 3A. In some embodiments, the PEgRNA comprises a spacer comprising a sequence as set forth in SEQ ID NOs. 969-970 of Table 3A. In some embodiments, the PEgRNA comprises a PBS comprising a sequence selected from the group consisting of SEQ Identifiers: B2511-B2519 of Table 3B. In some embodiments, the PEgRNA comprises a PBS comprising a sequence selected from the group consisting of SEQ ID NOs. 3166-3172 of Table 3B. In some embodiments, the PEgRNA comprises a PBS comprising a sequence selected from the group consisting of sequences TAAAAATA and TAAAAATAA as disclosed in Table 3B. In some embodiments, the PEgRNA comprises an editing template comprising a sequence selected from the group consisting of SEQ Identifiers: C12091-C12126 of Table 3C. In some embodiments, the PEgRNA comprises an editing template comprising a sequence selected from the group consisting of SEQ ID NOs:16117-16152 of Table 3C. In some embodiments, the prime editing composition further comprises a ngRNA that comprises a spacer comprising a sequence selected from the group consisting of SEQ Identifiers: D1435-D1440 of Table 3D. In some embodiments, the prime editing composition further comprises a ngRNA that comprises a spacer comprising a sequence selected from the group consisting of SEQ ID NOs. 21068-21073 of Table 3D.

In some embodiments, the PEgRNA comprises a spacer comprising the sequence of SEQ Identifier: A625 of Table 3A. In some embodiments, the PEgRNA comprises a spacer comprising a sequence as set forth in SEQ ID NO. 973 of Table 3A. In some embodiments, the PEgRNA comprises a PBS comprising a sequence selected from the group consisting of SEQ Identifiers: B2529-B2537 of Table 3B. In some embodiments, the PEgRNA comprises a PBS comprising a sequence selected from the group consisting of SEQ ID NOs. 3180-3186 of Table 3B. In some embodiments, the PEgRNA comprises a PBS comprising a sequence selected from the group consisting of sequences CAACAGAA and CAACAGAAG as disclosed in Table 3B. In some embodiments, the PEgRNA comprises an editing template comprising a sequence selected from the group consisting of SEQ Identifiers: C12127-C12141 of Table 3C. In some embodiments, the PEgRNA comprises an editing template comprising a sequence selected from the group consisting of SEQ ID NOs. 16153-16167 of Table 3C. In some embodiments, the prime editing composition further comprises a ngRNA that comprises a spacer comprising a sequence selected from the group consisting of SEQ Identifiers: D1445-D1461, D1473-D1478, D1516, and D1517 of Table 3D. In some embodiments, the prime editing composition further comprises a ngRNA that comprises a spacer comprising a sequence selected from the group consisting of SEQ ID NOs. 21078-21094, 21106-21111, 21149, and 21150 of Table 3D.

In some embodiments, the PEgRNA comprises a spacer comprising a sequence selected from the group consisting of SEQ Identifiers: A629-A630 of Table 3A. In some embodiments, the PEgRNA comprises a spacer comprising a sequence as set forth in SEQ ID NOs. 977-978 of Table 3A. In some embodiments, the PEgRNA comprises a PBS comprising a sequence selected from the group consisting of SEQ Identifiers: B2547-B2555 of Table 3B. In some embodiments, the PEgRNA comprises a PBS comprising a sequence selected from the group consisting of SEQ ID NOs. 3194-3200 of Table 3B. In some embodiments, the PEgRNA comprises a PBS comprising a sequence selected from the group consisting of sequences ATAAAAAT and ATAAAAATA as disclosed in Table 3B. In some embodiments, the PEgRNA comprises an editing template comprising a sequence selected from the group consisting of SEQ Identifiers: C12142-C12160 of Table 3C. In some embodiments, the PEgRNA comprises an editing template comprising a sequence selected from the group consisting of SEQ ID NOs. 16168-16186 of Table 3C. In some embodiments, the prime editing composition further comprises a ngRNA that comprises a spacer comprising a sequence selected from the group consisting of SEQ Identifiers: D1487-D1507 of Table 3D. In some embodiments, the prime editing composition further comprises a ngRNA that comprises a spacer comprising a sequence selected from the group consisting of SEQ ID NOs. 21120-21140 of Table 3D.

In some embodiments, the PEgRNA comprises a spacer comprising the sequence of SEQ Identifier A631 of Table 3A. In some embodiments, the PEgRNA comprises a spacer comprising a sequence as set forth in SEQ ID NO. 979 of Table 3A. In some embodiments, the PEgRNA comprises a PBS comprising a sequence selected from the group consisting of SEQ Identifiers: B2556-B2564 of Table 3B. In some embodiments, the PEgRNA comprises a PBS comprising a sequence selected from the group consisting of SEQ ID NOs. 3201-3207 of Table 3B. In some embodiments, the PEgRNA comprises a PBS comprising a sequence selected from the group consisting of sequences TTTTTGTT and TTTTTGTTT as disclosed in Table 3B. In some embodiments, the PEgRNA comprises an editing template comprising a sequence selected from the group consisting of SEQ Identifiers: C12161-C12164 of Table 3C. In some embodiments, the PEgRNA comprises an editing template comprising a sequence selected from the group consisting of SEQ ID NOs. 16187-16190 of Table 3C. In some embodiments, the prime editing composition further comprises a ngRNA that comprises a spacer comprising a sequence selected from the group consisting of SEQ Identifiers: D1487-D1504 of Table 3D. In some embodiments, the prime editing composition further comprises a ngRNA that comprises a spacer comprising a sequence selected from the group consisting of SEQ ID NOs. 21120-21137 of Table 3D.

In some embodiments, the PEgRNA comprises a spacer comprising a sequence selected from the group consisting of SEQ Identifiers: A645-A646 of Table 3A. In some embodiments, the PEgRNA comprises a spacer comprising a sequence as set forth in SEQ ID NOs. 993-994 of Table 3A. In some embodiments, the PEgRNA comprises a PBS comprising a sequence selected from the group consisting of SEQ Identifiers: B2637-B2645 of Table 3B. In some embodiments, the PEgRNA comprises a PBS comprising a sequence selected from the group consisting of SEQ ID NOs. 3264-3270 of Table 3B. In some embodiments, the PEgRNA comprises a PBS comprising a sequence selected from the group consisting of sequences TCACAGAT and TCACAGATC as disclosed in Table 3B. In some embodiments, the PEgRNA comprises an editing template comprising a sequence selected from the group consisting of SEQ Identifiers: C12165-C12226 of Table 3C. In some embodiments, the PEgRNA comprises an editing template comprising a sequence selected from the group consisting of SEQ ID NOs. 16191-16252 of Table 3C. In some embodiments, the prime editing composition further comprises a ngRNA that comprises a spacer comprising a sequence selected from the group consisting of SEQ Identifiers: D1435-D1440 of Table 3D. In some embodiments, the prime editing composition further comprises a ngRNA that comprises a spacer comprising a sequence selected from the group consisting of SEQ ID NOs. 21068-21073 of Table 3D.

In some embodiments, the PEgRNA comprises a spacer comprising the sequence selected from the group consisting of SEQ Identifier: A647 of Table 3A. In some embodiments, the PEgRNA comprises a spacer comprising a sequence as set forth in SEQ ID NO. 995 of Table 3A. In some embodiments, the PEgRNA comprises a PBS comprising a sequence selected from the group consisting of SEQ Identifiers: B2646-B2654 of Table 3B. In some embodiments, the PEgRNA comprises a PBS comprising a sequence selected from the group consisting of SEQ ID NOs. 3271-3277 of Table 3B. In some embodiments, the PEgRNA comprises a PBS comprising a sequence selected from the group consisting of sequences CTCACAGA and CTCACAGAT as disclosed in Table 3B. In some embodiments, the PEgRNA comprises an editing template comprising a sequence selected from the group consisting of SEQ Identifiers: C12227-C12257 of Table 3C. In some embodiments, the PEgRNA comprises an editing template comprising a sequence selected from the group consisting of SEQ ID NOs. 16253-16283 of Table 3C. In some embodiments, the prime editing composition further comprises a ngRNA that comprises a spacer comprising a sequence selected from the group consisting of SEQ Identifiers: D1493-D1504, and D1518-D1520 of Table 3D. In some embodiments, the prime editing composition further comprises a ngRNA that comprises a spacer comprising a sequence selected from the group consisting of SEQ ID NOs. 21126-21137, and 21151-21153 of Table 3D.

In some embodiments, the PEgRNA comprises a spacer comprising a sequence selected from the group consisting of SEQ Identifier: A632-A633 of Table 3A. In some embodiments, the PEgRNA comprises a spacer comprising a sequence as set forth in SEQ ID NOs. 980-981 of Table 3A. In some embodiments, the PEgRNA comprises a PBS comprising a sequence selected from the group consisting of SEQ Identifiers: B2565-B2573 of Table 3B. In some embodiments, the PEgRNA comprises a PBS comprising a sequence selected from the group consisting of SEQ ID NOs. 3208-3214 of Table 3B. In some embodiments, the PEgRNA comprises a PBS comprising a sequence selected from the group consisting of sequences ACATGCCA and ACATGCCAA as disclosed in Table 3B. In some embodiments, the PEgRNA comprises an editing template comprising a sequence selected from the group consisting of SEQ Identifiers: C12258-C12278 of Table 3C. In some embodiments, the PEgRNA comprises an editing template comprising a sequence selected from the group consisting of SEQ ID NOs. 16284-16304 of Table 3C. In some embodiments, the prime editing composition further comprises a ngRNA that comprises a spacer comprising a sequence selected from the group consisting of SEQ Identifiers: D1445-D1461, D1463-D1478, and D1516 of Table 3D. In some embodiments, the prime editing composition further comprises a ngRNA that comprises a spacer comprising a sequence selected from the group consisting of SEQ ID NOs. 21078-21094, 21096-21111, and 21149 of Table 3D.

In some embodiments, the PEgRNA comprises a spacer comprising a sequence selected from the group consisting of SEQ Identifier: A648-A650 of Table 3A. In some embodiments, the PEgRNA comprises a spacer comprising a sequence as set forth in SEQ ID NOs. 996-998 of Table 3A. In some embodiments, the PEgRNA comprises a PBS comprising a sequence selected from the group consisting of SEQ Identifiers: B2655-B2663 of Table 3B. In some embodiments, the PEgRNA comprises a PBS comprising a sequence selected from the group consisting of SEQ ID NOs. 3278-3284 of Table 3B. In some embodiments, the PEgRNA comprises a PBS comprising a sequence selected from the group consisting of sequences AAGGTAAA and AAGGTAAAC as disclosed in Table 3B. In some embodiments, the PEgRNA comprises an editing template comprising a sequence selected from the group consisting of SEQ Identifiers: C12279-C12287 of Table 3C. In some embodiments, the PEgRNA comprises an editing template comprising a sequence selected from the group consisting of SEQ ID NOs. 16305-16313 of Table 3C. In some embodiments, the prime editing composition further comprises a ngRNA that comprises a spacer comprising a sequence selected from the group consisting of SEQ Identifiers: D1475, D1476, D1477, D1479, D1480, D1482, D1483, D1485, D1486, D1515, and D1521-D1528 of Table 3D. In some embodiments, the prime editing composition further comprises a ngRNA that comprises a spacer comprising a sequence selected from the group consisting of SEQ ID NOs. 21108, 21109, 21110, 21112, 21113, 21115, 21116, 21118, 21119, 21148, and 21154-21161 of Table 3D.

In some embodiments, the PEgRNA comprises a spacer comprising the sequence of SEQ Identifier: A651 of Table 3A. In some embodiments, the PEgRNA comprises a spacer comprising a sequence as set forth in SEQ ID NO. 999 of Table 3A. In some embodiments, the PEgRNA comprises a PBS comprising a sequence selected from the group consisting of SEQ Identifiers: B2664-B2672 of Table 3B. In some embodiments, the PEgRNA comprises a PBS comprising a sequence selected from the group consisting of SEQ ID NOs. 3285-3291 of Table 3B. In some embodiments, the PEgRNA comprises a PBS comprising a sequence selected from the group consisting of sequences GGTAAACC and GGTAAACCT as disclosed in Table 3B. In some embodiments, the PEgRNA comprises an editing template comprising a sequence selected from the group consisting of SEQ Identifiers: C12288-C12294 of Table 3C. In some embodiments, the PEgRNA comprises an editing template comprising a sequence selected from the group consisting of SEQ ID NOs. 16314-16320 of Table 3C. In some embodiments, the prime editing composition further comprises a ngRNA that comprises a spacer comprising a sequence selected from the group consisting of SEQ Identifiers: D1448-D1461, D1473-D1478, D1516, D1517, and D1529 of Table 3D. In some embodiments, the prime editing composition further comprises a ngRNA that comprises a spacer comprising a sequence selected from the group consisting of SEQ ID NOs. 21081-21094, 21106-21111, 21149, 21150, and 21162 of Table 3D.

In some embodiments, the PEgRNA comprises a spacer comprising the sequence of SEQ Identifier: A634 of Table 3A. In some embodiments, the PEgRNA comprises a spacer comprising a sequence as set forth in SEQ ID NO. 982 of Table 3A. In some embodiments, the PEgRNA comprises a PBS comprising a sequence selected from the group consisting of SEQ Identifiers: B2574-B2582 of Table 3B. In some embodiments, the PEgRNA comprises a PBS comprising a sequence selected from the group consisting of SEQ ID NOs. 3215-3221 of Table 3B. In some embodiments, the PEgRNA comprises a PBS comprising a sequence selected from the group consisting of sequences TTGTTTTT and TTGTTTTTA as disclosed in Table 3B. In some embodiments, the PEgRNA comprises an editing template comprising the sequence of SEQ Identifier: C12295 of Table 3C. In some embodiments, the PEgRNA comprises an editing template comprising a sequence selected from the group consisting of SEQ ID NO. 16321 of Table 3C. In some embodiments, the prime editing composition further comprises a ngRNA that comprises a spacer comprising a sequence selected from the group consisting of SEQ Identifiers: D1487-D1503 of Table 3D. In some embodiments, the prime editing composition further comprises a ngRNA that comprises a spacer comprising a sequence selected from the group consisting of SEQ ID NOs. 21120-21136 of Table 3D.

In some embodiments, the PEgRNA comprises a spacer comprising a sequence selected from the group consisting of SEQ Identifiers: A635-A636 of Table 3A. In some embodiments, the PEgRNA comprises a spacer comprising a sequence as set forth in SEQ ID NOs. 983-984 of Table 3A. In some embodiments, the PEgRNA comprises a PBS comprising a sequence selected from the group consisting of SEQ Identifiers: B2583-B2591 of Table 3B. In some embodiments, the PEgRNA comprises a PBS comprising a sequence selected from the group consisting of SEQ ID NOs. 3222-3228 of Table 3B. In some embodiments, the PEgRNA comprises a PBS comprising a sequence selected from the group consisting of sequences AGTTCATT and AGTTCATTG as disclosed in Table 3B. In some embodiments, the PEgRNA comprises an editing template comprising a sequence selected from the group consisting of SEQ Identifiers: C12296-C12325 of Table 3C. In some embodiments, the PEgRNA comprises an editing template comprising a sequence selected from the group consisting of SEQ ID NOs. 16322-16351 of Table 3C. In some embodiments, the prime editing composition further comprises a ngRNA that comprises a spacer comprising a sequence selected from the group consisting of SEQ Identifiers: D1444-D1461, D1463-D1474, D1530, and D1531 of Table 3D. In some embodiments, the prime editing composition further comprises a ngRNA that comprises a spacer comprising a sequence selected from the group consisting of SEQ ID NOs. 21077-21094, 21096-21107, 21163, and 21164 of Table 3D.

In some embodiments, the PEgRNA comprises a spacer comprising a sequence selected from the group consisting of SEQ Identifiers: A637-A638 of Table 3A. In some embodiments, the PEgRNA comprises a spacer comprising a sequence as set forth in SEQ ID NOs. 985-986 of Table 3A. In some embodiments, the PEgRNA comprises a PBS comprising a sequence selected from the group consisting of SEQ Identifiers: B2592-B2600 of Table 3B. In some embodiments, the PEgRNA comprises a PBS comprising a sequence selected from the group consisting of SEQ ID NOs. 3229-3235 of Table 3B. In some embodiments, the PEgRNA comprises a PBS comprising a sequence selected from the group consisting of sequences CTTTAAGT and CTTTAAGTT as disclosed in Table 3B. In some embodiments, the PEgRNA comprises an editing template comprising a sequence selected from the group consisting of SEQ Identifiers: C12326-C12356 of Table 3C. In some embodiments, the PEgRNA comprises an editing template comprising a sequence selected from the group consisting of SEQ ID NOs. 16352-16382 of Table 3C. In some embodiments, the prime editing composition further comprises a ngRNA that comprises a spacer comprising a sequence selected from the group consisting of SEQ Identifiers: D1416-D1423, D1426-1433, D1532, and D1533 of Table 3D. In some embodiments, the prime editing composition further comprises a ngRNA that comprises a spacer comprising a sequence selected from the group consisting of SEQ ID NOs. 21049-21056, 21059-21066, 21165, and 21166 of Table 3D.

In some embodiments, the PEgRNA comprises a spacer comprising the sequence of SEQ Identifier: A639 of Table 3A. In some embodiments, the PEgRNA comprises a spacer comprising a sequence as set forth in SEQ ID NO. 987 of Table 3A. In some embodiments, the PEgRNA comprises a PBS comprising a sequence selected from the group consisting of SEQ Identifiers: B2601-B2609 of Table 3B. In some embodiments, the PEgRNA comprises a PBS comprising a sequence selected from the group consisting of SEQ ID NOs. 3236-3242 of Table 3B. In some embodiments, the PEgRNA comprises a PBS comprising a sequence selected from the group consisting of sequences TCTTTAAG and TCTTTAAGT as disclosed in Table 3B. In some embodiments, the PEgRNA comprises an editing template comprising a sequence selected from the group consisting of SEQ Identifiers: C12357-C12387 of Table 3C. In some embodiments, the PEgRNA comprises an editing template comprising a sequence selected from the group consisting of SEQ ID NOs. 16383-16413 of Table 3C. In some embodiments, the prime editing composition further comprises a ngRNA that comprises a spacer comprising a sequence selected from the group consisting of SEQ Identifiers: D1444-D1461, D1473, D1474, D1531, and D1534 of Table 3D. In some embodiments, the prime editing composition further comprises a ngRNA that comprises a spacer comprising a sequence selected from the group consisting of SEQ ID NOs. 21077-21094, 21106, 21107, 21164, and 21167 of Table 3D.

In some embodiments, the PEgRNA comprises a spacer comprising a sequence selected from the group consisting of SEQ Identifiers: A640-A641 of Table 3A. In some embodiments, the PEgRNA comprises a spacer comprising a sequence as set forth in SEQ ID NOs. 988-989 of Table 3A. In some embodiments, the PEgRNA comprises a PBS comprising a sequence selected from the group consisting of SEQ Identifiers: B2610-B2618 of Table 3B. In some embodiments, the PEgRNA comprises a PBS comprising a sequence selected from the group consisting of SEQ ID NOs. 3243-3249 of Table 3B. In some embodiments, the PEgRNA comprises a PBS comprising a sequence selected from the group consisting of sequences CTGAAATA and CTGAAATAA as disclosed in Table 3B. In some embodiments, the PEgRNA comprises an editing template comprising a sequence selected from the group consisting of SEQ Identifiers: C12388-C12411 of Table 3C. In some embodiments, the PEgRNA comprises an editing template comprising a sequence selected from the group consisting of SEQ ID NOs. 16414-16437 of Table 3C. In some embodiments, the prime editing composition further comprises a ngRNA that comprises a spacer comprising a sequence selected from the group consisting of SEQ Identifiers: D1489-D1507 of Table 3D. In some embodiments, the prime editing composition further comprises a ngRNA that comprises a spacer comprising a sequence selected from the group consisting of SEQ ID NOs. 21122-21140 of Table 3D.

In some embodiments, the PEgRNA comprises a spacer comprising the of SEQ Identifier: A642 of Table 3A. In some embodiments, the PEgRNA comprises a spacer comprising a sequence as set forth in SEQ ID NO. 990 of Table 3A. In some embodiments, the PEgRNA comprises a PBS comprising a sequence selected from the group consisting of SEQ Identifiers: B2619-B2627 of Table 3B. In some embodiments, the PEgRNA comprises a PBS comprising a sequence selected from the group consisting of SEQ ID NOs. 3250-3256 of Table 3B. In some embodiments, the PEgRNA comprises a PBS comprising a sequence selected from the group consisting of sequences GAAATAAA and GAAATAAAA as disclosed in Table 3B. In some embodiments, the PEgRNA comprises an editing template comprising a sequence selected from the group consisting of SEQ Identifiers: C12412-C12433 of Table 3C. In some embodiments, the PEgRNA comprises an editing template comprising a sequence selected from the group consisting of SEQ ID NOs. 16438-16459 of Table 3C. In some embodiments, the prime editing composition further comprises a ngRNA that comprises a spacer comprising a sequence selected from the group consisting of SEQ Identifiers: D1487-D1504 of Table 3D. In some embodiments, the prime editing composition further comprises a ngRNA that comprises a spacer comprising a sequence selected from the group consisting of SEQ ID NOs. 21120-21137 of Table 3D.

In some embodiments, the PEgRNA comprises a spacer comprising a sequence selected from the group consisting of SEQ Identifiers: A652-A653 of Table 3A. In some embodiments, the PEgRNA comprises a spacer comprising a sequence as set forth in SEQ ID NOs. 1000-1001 of Table 3A. In some embodiments, the PEgRNA comprises a PBS comprising a sequence selected from the group consisting of SEQ Identifiers: B2673-B2681 of Table 3B. In some embodiments, the PEgRNA comprises a PBS comprising a sequence selected from the group consisting of SEQ ID NOs. 3292-3298 of Table 3B. In some embodiments, the PEgRNA comprises a PBS comprising a sequence selected from the group consisting of sequences GATCGCAT and GATCGCATC as disclosed in Table 3B. In some embodiments, the PEgRNA comprises an editing template comprising a sequence selected from the group consisting of SEQ Identifiers: C12434-C12495 of Table 3C. In some embodiments, the PEgRNA comprises an editing template comprising a sequence selected from the group consisting of SEQ ID NOs. 16460-16521 of Table 3C. In some embodiments, the prime editing composition further comprises a ngRNA that comprises a spacer comprising a sequence selected from the group consisting of SEQ Identifiers: D1435-D1440 of Table 3D. In some embodiments, the prime editing composition further comprises a ngRNA that comprises a spacer comprising a sequence selected from the group consisting of SEQ ID NOs. 21068-21073 of Table 3D.

In some embodiments, the PEgRNA comprises a spacer comprising the sequence of SEQ Identifier: A654 of Table 3A. In some embodiments, the PEgRNA comprises a spacer comprising a sequence as set forth in SEQ ID NO. 1002 of Table 3A. In some embodiments, the PEgRNA comprises a PBS comprising a sequence selected from the group consisting of SEQ Identifiers: B2682-B2690 of Table 3B. In some embodiments, the PEgRNA comprises a PBS comprising a sequence selected from the group consisting of SEQ ID NOs. 3299-3305 of Table 3B. In some embodiments, the PEgRNA comprises a PBS comprising a sequence selected from the group consisting of sequences AGATCGCA and AGATCGCAT as disclosed in Table 3B. In some embodiments, the PEgRNA comprises an editing template comprising a sequence selected from the group consisting of SEQ Identifiers: C12496-C12526 of Table 3C. In some embodiments, the PEgRNA comprises an editing template comprising a sequence selected from the group consisting of SEQ ID NOs. 16522-16552 of Table 3C. In some embodiments, the prime editing composition further comprises a ngRNA that comprises a spacer comprising a sequence selected from the group consisting of SEQ Identifiers: D1491-D1504, D1518, and D1519 of Table 3D. In some embodiments, the prime editing composition further comprises a ngRNA that comprises a spacer comprising a sequence selected from the group consisting of SEQ ID NOs. 21124-21137, 21151, and 21152 of Table 3D.

In some embodiments, the PEgRNA comprises a spacer comprising a sequence selected from the group consisting of SEQ Identifiers: A655-A656 of Table 3A. In some embodiments, the PEgRNA comprises a spacer comprising a sequence as set forth in SEQ ID NOs. 1003-1004 of Table 3A. In some embodiments, the PEgRNA comprises a PBS comprising a sequence selected from the group consisting of SEQ Identifiers: B2691-B2699 of Table 3B. In some embodiments, the PEgRNA comprises a PBS comprising a sequence selected from the group consisting of SEQ ID NOs. 3306-3312 of Table 3B. In some embodiments, the PEgRNA comprises a PBS comprising a sequence selected from the group consisting of sequences CACAGATC and CACAGATCG as disclosed in Table 3B. In some embodiments, the PEgRNA comprises an editing template comprising a sequence selected from the group consisting of SEQ Identifiers: C12527-C12557 of Table 3C. In some embodiments, the PEgRNA comprises an editing template comprising a sequence selected from the group consisting of SEQ ID NOs. 16553-16583 of Table 3C. In some embodiments, the prime editing composition further comprises a ngRNA that comprises a spacer comprising a sequence selected from the group consisting of SEQ Identifiers: D1493-D1507, D1518, D1519, and D1535 of Table 3D. In some embodiments, the prime editing composition further comprises a ngRNA that comprises a spacer comprising a sequence selected from the group consisting of SEQ ID NOs. 21126-21140, 21151, 21152, and 21168 of Table 3D.

In some embodiments, the PEgRNA comprises a spacer comprising a sequence selected from the group consisting of SEQ Identifiers: A643-A644 of Table 3A. In some embodiments, the PEgRNA comprises a spacer comprising a sequence as set forth in SEQ ID NOs. 991-992 of Table 3A. In some embodiments, the PEgRNA comprises a PBS comprising a sequence selected from the group consisting of SEQ Identifiers: B2628-B2636 of Table 3B. In some embodiments, the PEgRNA comprises a PBS comprising a sequence selected from the group consisting of SEQ ID NOs. 3257-3263 of Table 3B. In some embodiments, the PEgRNA comprises a PBS comprising a sequence selected from the group consisting of sequences GTTCATTG and GTTCATTGA as disclosed in Table 3B. In some embodiments, the PEgRNA comprises an editing template comprising a sequence selected from the group consisting of SEQ Identifiers: C12558-C12615 of Table 3C. In some embodiments, the PEgRNA comprises an editing template comprising a sequence selected from the group consisting of SEQ ID NOs: of Table 3C. In some embodiments, the prime editing composition further comprises a ngRNA that comprises a spacer comprising a sequence selected from the group consisting of SEQ Identifiers: D1416-D1423, D1427-D1433, D1514, and D1515 of Table 3D. In some embodiments, the prime editing composition further comprises a ngRNA that comprises a spacer comprising a sequence selected from the group consisting of SEQ ID NOs. 21049-21056, 21060-21066, 21147, and 21148 of Table 3D.

In some embodiments, the PEgRNA comprises a spacer comprising a sequence selected from the group consisting of SEQ Identifiers: A657-A658 of Table 3A. In some embodiments, the PEgRNA comprises a spacer comprising a sequence as set forth in SEQ ID NOs. 1005-1006 of Table 3A. In some embodiments, the PEgRNA comprises a PBS comprising a sequence selected from the group consisting of SEQ Identifiers: B2700-B2708 of Table 3B. In some embodiments, the PEgRNA comprises a PBS comprising a sequence selected from the group consisting of SEQ ID NOs. 3313-3319 of Table 3B. In some embodiments, the PEgRNA comprises a PBS comprising a sequence selected from the group consisting of sequences GAAGGTAA and GAAGGTAAA as disclosed in Table 3B. In some embodiments, the PEgRNA comprises an editing template comprising a sequence selected from the group consisting of SEQ Identifiers: C12616-C12625 of Table 3C. In some embodiments, the PEgRNA comprises an editing template comprising a sequence selected from the group consisting of SEQ ID NOs: of Table 3C. In some embodiments, the prime editing composition further comprises a ngRNA that comprises a spacer comprising a sequence selected from the group consisting of SEQ Identifiers: D1448-D1461, D1473-D1478, D1512-D1513, D1516, D1517, D1529, and D1536-D1539 of Table 3D. In some embodiments, the prime editing composition further comprises a ngRNA that comprises a spacer comprising a sequence selected from the group consisting of SEQ ID NOs. 21081-21094, 21106-21111, 21145-21146, 21149, 21150, 21162 of Table 3D.

In some embodiments, the PEgRNA comprises a spacer comprising the of SEQ Identifier: A659 of Table 3A. In some embodiments, the PEgRNA comprises a spacer comprising a sequence as set forth in SEQ ID NO. 1007 of Table 3A. In some embodiments, the PEgRNA comprises a PBS comprising a sequence selected from the group consisting of SEQ Identifiers: B2709-B2717 of Table 3B. In some embodiments, the PEgRNA comprises a PBS comprising a sequence selected from the group consisting of SEQ ID NOs. 3320-3326 of Table 3B. In some embodiments, the PEgRNA comprises a PBS comprising a sequence selected from the group consisting of sequences AGAAGGTA and AGAAGGTAA as disclosed in Table 3B. In some embodiments, the PEgRNA comprises an editing template comprising a sequence selected from the group consisting of SEQ Identifiers: C12626-C12636 of Table 3C. In some embodiments, the PEgRNA comprises an editing template comprising a sequence selected from the group consisting of SEQ ID NOs. 16652-16662 of Table 3C. In some embodiments, the prime editing composition further comprises a ngRNA that comprises a spacer comprising a sequence selected from the group consisting of SEQ Identifiers: D1447-D1461, D1473-D1478, D1516, D1517, and D1529 of Table 3D. In some embodiments, the prime editing composition further comprises a ngRNA that comprises a spacer comprising a sequence selected from the group consisting of SEQ ID NOs. 21080-21094, 21106-21111, 21149, 21150, and 21162 of Table 3D.

In some embodiments, the PEgRNA comprises a spacer comprising a sequence selected from the group consisting of SEQ Identifiers: A660-A661 of Table 3A. In some embodiments, the PEgRNA comprises a spacer comprising a sequence as set forth in SEQ ID NOs. 1008-1009 of Table 3A. In some embodiments, the PEgRNA comprises a PBS comprising a sequence selected from the group consisting of SEQ Identifiers: B2718-B2726 of Table 3B. In some embodiments, the PEgRNA comprises a PBS comprising a sequence selected from the group consisting of SEQ ID NOs. 3327-3333 of Table 3B. In some embodiments, the PEgRNA comprises a PBS comprising a sequence selected from the group consisting of sequences ATCGCATC and ATCGCATCT as disclosed in Table 3B. In some embodiments, the PEgRNA comprises an editing template comprising a sequence selected from the group consisting of SEQ Identifiers: C12637-C12667 of Table 3C. In some embodiments, the PEgRNA comprises an editing template comprising a sequence selected from the group consisting of SEQ ID NOs. 16663-16693 of Table 3C. In some embodiments, the prime editing composition further comprises a ngRNA that comprises a spacer comprising a sequence selected from the group consisting of SEQ Identifiers: D1491-D1507, D1518, and D1535 of Table 3D. In some embodiments, the prime editing composition further comprises a ngRNA that comprises a spacer comprising a sequence selected from the group consisting of SEQ ID NOs. 21124-21140, 21151, and 21168 of Table 3D.

In some embodiments, the PEgRNA comprises a spacer comprising a sequence selected from the group consisting of SEQ Identifiers: A662-A664 of Table 3A. In some embodiments, the PEgRNA comprises a spacer comprising a sequence as set forth in SEQ ID NOs. 1010-1012 of Table 3A. In some embodiments, the PEgRNA comprises a PBS comprising a sequence selected from the group consisting of SEQ Identifiers: B2727-B2735 of Table 3B. In some embodiments, the PEgRNA comprises a PBS comprising a sequence selected from the group consisting of SEQ ID NOs. 3334-3340 of Table 3B. In some embodiments, the PEgRNA comprises a PBS comprising a sequence selected from the group consisting of sequences ACAGATCG and ACAGATCGC as disclosed in Table 3B. In some embodiments, the PEgRNA comprises an editing template comprising a sequence selected from the group consisting of SEQ Identifiers: C12668-C12698 of Table 3C. In some embodiments, the PEgRNA comprises an editing template comprising a sequence selected from the group consisting of SEQ ID NOs: 16694-16724 of Table 3C. In some embodiments, the prime editing composition further comprises a ngRNA that comprises a spacer comprising a sequence selected from the group consisting of SEQ Identifiers: D1437, D1540, and D1541 of Table 3D. In some embodiments, the prime editing composition further comprises a ngRNA that comprises a spacer comprising a sequence selected from the group consisting of SEQ ID NOs. 21070, 21173, and 21174 of Table 3D.

In some embodiments, the PEgRNA comprises a spacer comprising the of SEQ Identifier: A665 of Table 3A. In some embodiments, the PEgRNA comprises a spacer comprising a sequence as set forth in SEQ ID NO. 1013 of Table 3A. In some embodiments, the PEgRNA comprises a PBS comprising a sequence selected from the group consisting of SEQ Identifiers: B2736-B2744 of Table 3B. In some embodiments, the PEgRNA comprises a PBS comprising a sequence selected from the group consisting of SEQ ID NOs. 3341-3347 of Table 3B. In some embodiments, the PEgRNA comprises a PBS comprising a sequence selected from the group consisting of sequences CGCATCTG and CGCATCTGA as disclosed in Table 3B. In some embodiments, the PEgRNA comprises an editing template comprising a sequence selected from the group consisting of SEQ Identifiers: C12699-C12727 of Table 3C. In some embodiments, the PEgRNA comprises an editing template comprising a sequence selected from the group consisting of SEQ ID NOs. 16725-16753 of Table 3C. In some embodiments, the prime editing composition further comprises a ngRNA that comprises a spacer comprising a sequence selected from the group consisting of SEQ Identifiers: D1491-D1504 and D1518 of Table 3D. In some embodiments, the prime editing composition further comprises a ngRNA that comprises a spacer comprising a sequence selected from the group consisting of SEQ ID NOs. 21124-21137 and 21151 of Table 3D.

In some embodiments, the PEgRNA comprises a spacer comprising a sequence selected from the group consisting of SEQ Identifiers: A621-A622 of Table 3A. In some embodiments, the PEgRNA comprises a spacer comprising a sequence as set forth in SEQ ID NOs. 969-970 of Table 3A. In some embodiments, the PEgRNA comprises a PBS comprising a sequence selected from the group consisting of SEQ Identifiers: B2511-B2519 of Table 3B. In some embodiments, the PEgRNA comprises a PBS comprising a sequence selected from the group consisting of SEQ ID NOs. 3166-3172 of Table 3B. In some embodiments, the PEgRNA comprises a PBS comprising a sequence selected from the group consisting of sequences TAAAAATA and TAAAAATAA as disclosed in Table 3B. In some embodiments, the PEgRNA comprises an editing template comprising a sequence selected from the group consisting of SEQ Identifiers: C12728-C12761 of Table 3C. In some embodiments, the PEgRNA comprises an editing template comprising a sequence selected from the group consisting of SEQ ID NOs. 16754-16787 of Table 3C. In some embodiments, the prime editing composition further comprises a ngRNA that comprises a spacer comprising a sequence selected from the group consisting of SEQ Identifiers: D1435-D1440 of Table 3D. In some embodiments, the prime editing composition further comprises a ngRNA that comprises a spacer comprising a sequence selected from the group consisting of SEQ ID NOs. 21068-21073 of Table 3D.

In some embodiments, the PEgRNA comprises a spacer comprising the sequence of SEQ Identifier: A625 of Table 3A. In some embodiments, the PEgRNA comprises a spacer comprising a sequence as set forth in SEQ ID NO. 973 of Table 3A. In some embodiments, the PEgRNA comprises a PBS comprising a sequence selected from the group consisting of SEQ Identifiers: B2529-B2537 of Table 3B. In some embodiments, the PEgRNA comprises a PBS comprising a sequence selected from the group consisting of SEQ ID NOs. 3180-3186 of Table 3B. In some embodiments, the PEgRNA comprises a PBS comprising a sequence selected from the group consisting of sequences CAACAGAA and CAACAGAAG as disclosed in Table 3B. In some embodiments, the PEgRNA comprises an editing template comprising a sequence selected from the group consisting of SEQ Identifiers: C12762-C12777 of Table 3C. In some embodiments, the PEgRNA comprises an editing template comprising a sequence selected from the group consisting of SEQ ID NOs. 16788-16803 of Table 3C. In some embodiments, the prime editing composition further comprises a ngRNA that comprises a spacer comprising a sequence selected from the group consisting of SEQ Identifiers: D1445-D1461, D1473-D1478, D1516, and D1517 of Table 3D. In some embodiments, the prime editing composition further comprises a ngRNA that comprises a spacer comprising a sequence selected from the group consisting of SEQ ID NOs. 21078-21094, 21106-21111, 21149, and 21150 of Table 3D.

In some embodiments, the PEgRNA comprises a spacer comprising a sequence selected from the group consisting of SEQ Identifiers: A629-A630 of Table 3A. In some embodiments, the PEgRNA comprises a spacer comprising a sequence as set forth in SEQ ID NOs. 977-978 of Table 3A. In some embodiments, the PEgRNA comprises a PBS comprising a sequence selected from the group consisting of SEQ Identifiers: B2547-B2555 of Table 3B. In some embodiments, the PEgRNA comprises a PBS comprising a sequence selected from the group consisting of SEQ ID NOs. 3194-3200 of Table 3B. In some embodiments, the PEgRNA comprises a PBS comprising a sequence selected from the group consisting of sequences ATAAAAAT and ATAAAAATA as disclosed in Table 3B. In some embodiments, the PEgRNA comprises an editing template comprising a sequence selected from the group consisting of SEQ Identifiers: C12778-C12795 of Table 3C. In some embodiments, the PEgRNA comprises an editing template comprising a sequence selected from the group consisting of SEQ ID NOs. 16804-16821 of Table 3C. In some embodiments, the prime editing composition further comprises a ngRNA that comprises a spacer comprising a sequence selected from the group consisting of SEQ Identifiers: D1487-D1507 of Table 3D. In some embodiments, the prime editing composition further comprises a ngRNA that comprises a spacer comprising a sequence selected from the group consisting of SEQ ID NOs. 21120-21140 of Table 3D.

In some embodiments, the PEgRNA comprises a spacer comprising the of SEQ Identifier: A631 of Table 3A. In some embodiments, the PEgRNA comprises a spacer comprising a sequence as set forth in SEQ ID NO.979 of Table 3A. In some embodiments, the PEgRNA comprises a PBS comprising a sequence selected from the group consisting of SEQ Identifiers: B2556-B2564 of Table 3B. In some embodiments, the PEgRNA comprises a PBS comprising a sequence selected from the group consisting of SEQ ID NOs. 3201-3207 of Table 3B. In some embodiments, the PEgRNA comprises a PBS comprising a sequence selected from the group consisting of sequences TTTTTGTT and TTTTTGTTT as disclosed in Table 3B. In some embodiments, the PEgRNA comprises an editing template comprising a sequence selected from the group consisting of SEQ Identifiers: C12796-C12798 of Table 3C. In some embodiments, the PEgRNA comprises an editing template comprising a sequence selected from the group consisting of SEQ ID NOs. 16822-16824 of Table 3C. In some embodiments, the prime editing composition further comprises a ngRNA that comprises a spacer comprising a sequence selected from the group consisting of SEQ Identifiers: D1487-D1504 of Table 3D. In some embodiments, the prime editing composition further comprises a ngRNA that comprises a spacer comprising a sequence selected from the group consisting of SEQ ID NOs. 21120-21137 of Table 3D.

In some embodiments, the PEgRNA comprises a spacer comprising a sequence selected from the group consisting of SEQ Identifiers: A666-A667 of Table 3A. In some embodiments, the PEgRNA comprises a spacer comprising a sequence as set forth in SEQ ID NOs. 1014-1015 of Table 3A. In some embodiments, the PEgRNA comprises a PBS comprising a sequence selected from the group consisting of SEQ Identifiers: B2637-B2645 of Table 3B. In some embodiments, the PEgRNA comprises a PBS comprising a sequence selected from the group consisting of SEQ ID NOs. 3264-3270 of Table 3B. In some embodiments, the PEgRNA comprises a PBS comprising a sequence selected from the group consisting of sequences TCACAGAT and TCACAGATC as disclosed in Table 3B. In some embodiments, the PEgRNA comprises an editing template comprising a sequence selected from the group consisting of SEQ Identifiers: C12165, C12167, C12170, C12172, C12173, C12175, C12177, C12180, C12181, C12184, C12186, C12188, C12189, C12192, C12193, C12195, C12198, C12200, C12201, C12203, C12206, C12208, C12210, C12212, C12214, C12215, C12218, C12219, C12222, C12224, C12226, C12799, C12800, C12801, C12802, C12803, C12804, C12805, C12806, C12807, C12808, C12809, C12810, C12811, C12812, C12813, C12814, C12815, C12816, C12817, C12818, C12819, C12820, C12821, C12822, C12823, C12824, C12825, C12826, C12827, C12828, and C12829 of Table 3C. In some embodiments, the PEgRNA comprises an editing template comprising a sequence selected from the group consisting of SEQ ID NOs. 16191, 16193, 16196, 16198, 16194, 16201, 16203, 16206, 16207, 16210, 16212, 16214, 16215, 16218, 16219, 16220, 16224, 16226, 16227, 16230, 16232, 16234, 16236, 16238, 16240, 16241, 16244, 16245, 16248, 16250, 16252, and 16825-16855 of Table 3C. In some embodiments, the prime editing composition further comprises a ngRNA that comprises a spacer comprising a sequence selected from the group consisting of SEQ Identifiers: D1435-D1440 of Table 3D. In some embodiments, the prime editing composition further comprises a ngRNA that comprises a spacer comprising a sequence selected from the group consisting of SEQ ID NOs. 21068-21073 of Table 3D.

In some embodiments, the PEgRNA comprises a spacer comprising the sequence of SEQ Identifier: A668 of Table 3A. In some embodiments, the PEgRNA comprises a spacer comprising a sequence as set forth in SEQ ID NO. 1016 of Table 3A. In some embodiments, the PEgRNA comprises a PBS comprising a sequence selected from the group consisting of SEQ Identifiers: B2646-B2654 of Table 3B. In some embodiments, the PEgRNA comprises a PBS comprising a sequence selected from the group consisting of SEQ ID NOs. 3271-3277 of Table 3B. In some embodiments, the PEgRNA comprises a PBS comprising a sequence selected from the group consisting of sequences CTCACAGA and CTCACAGAT as disclosed in Table 3B. In some embodiments, the PEgRNA comprises an editing template comprising a sequence selected from the group consisting of SEQ Identifiers: C12830-C12860 of Table 3C. In some embodiments, the PEgRNA comprises an editing template comprising a sequence selected from the group consisting of SEQ ID NOs. 16856-16886 of Table 3C. In some embodiments, the prime editing composition further comprises a ngRNA that comprises a spacer comprising a sequence selected from the group consisting of SEQ Identifiers: D1493-D1504, D1542, D1543, and D1544 of Table 3D. In some embodiments, the prime editing composition further comprises a ngRNA that comprises a spacer comprising a sequence selected from the group consisting of SEQ ID NOs. 21126-21137, 21175, 21176, and 21177 of Table 3D.

In some embodiments, the PEgRNA comprises a spacer comprising a sequence selected from the group consisting of SEQ Identifiers: A632-A633 of Table 3A. In some embodiments, the PEgRNA comprises a spacer comprising a sequence as set forth in SEQ ID NOs. 980-981 of Table 3A. In some embodiments, the PEgRNA comprises a PBS comprising a sequence selected from the group consisting of SEQ Identifiers: B2565-B2573 of Table 3B. In some embodiments, the PEgRNA comprises a PBS comprising a sequence selected from the group consisting of SEQ ID NOs. 3208-3214 of Table 3B. In some embodiments, the PEgRNA comprises a PBS comprising a sequence selected from the group consisting of sequences ACATGCCA and ACATGCCAA as disclosed in Table 3B. In some embodiments, the PEgRNA comprises an editing template comprising a sequence selected from the group consisting of SEQ Identifiers: C12861-C12882 of Table 3C. In some embodiments, the PEgRNA comprises an editing template comprising a sequence selected from the group consisting of SEQ ID NOs. 16887-16908 of Table 3C. In some embodiments, the prime editing composition further comprises a ngRNA that comprises a spacer comprising a sequence selected from the group consisting of SEQ Identifiers: D1445-D1461, D1463-1478, and D1516 of Table 3D. In some embodiments, the prime editing composition further comprises a ngRNA that comprises a spacer comprising a sequence selected from the group consisting of SEQ ID NOs. 21078-21094, 21096-21111, and 21149 of Table 3D.

In some embodiments, the PEgRNA comprises a spacer comprising a sequence selected from the group consisting of SEQ Identifiers: A648-A650 of Table 3A. In some embodiments, the PEgRNA comprises a spacer comprising a sequence as set forth in SEQ ID NOs. 996-998 of Table 3A. In some embodiments, the PEgRNA comprises a PBS comprising a sequence selected from the group consisting of SEQ Identifiers B2655-B2663 of Table 3B. In some embodiments, the PEgRNA comprises a PBS comprising a sequence selected from the group consisting of SEQ ID NOs. 3278-3284 of Table 3B. In some embodiments, the PEgRNA comprises a PBS comprising a sequence selected from the group consisting of sequences AAGGTAAA and AAGGTAAAC as disclosed in Table 3B. In some embodiments, the PEgRNA comprises an editing template comprising a sequence selected from the group consisting of SEQ Identifiers: C12883-C12892 of Table 3C. In some embodiments, the PEgRNA comprises an editing template comprising a sequence selected from the group consisting of SEQ ID NOs. 16909-16918 of Table 3C. In some embodiments, the prime editing composition further comprises a ngRNA that comprises a spacer comprising a sequence selected from the group consisting of SEQ Identifiers: D1475, D1476, D1477, D1479, D1480, D1482, D1483, D1485, D1486, D1515, and D1521-D1528 of Table 3D. In some embodiments, the prime editing composition further comprises a ngRNA that comprises a spacer comprising a sequence selected from the group consisting of SEQ ID NOs. 21108, 21109, 21110, 21112, 21113, 21115, 21116, 21118, 21119, 21148, 21154-21161 of Table 3D.

In some embodiments, the PEgRNA comprises a spacer comprising the of SEQ Identifier: A651 of Table 3A. In some embodiments, the PEgRNA comprises a spacer comprising a sequence as set forth in SEQ ID NO. 999 of Table 3A. In some embodiments, the PEgRNA comprises a PBS comprising a sequence selected from the group consisting of SEQ Identifiers: B2664-B2672 of Table 3B. In some embodiments, the PEgRNA comprises a PBS comprising a sequence selected from the group consisting of SEQ ID NOs. 3285-3291 of Table 3B. In some embodiments, the PEgRNA comprises a PBS comprising a sequence selected from the group consisting of sequences GGTAAACC and GGTAAACCT as disclosed in Table 3B. In some embodiments, the PEgRNA comprises an editing template comprising a sequence selected from the group consisting of SEQ Identifiers: C12893-C12900 of Table 3C. In some embodiments, the PEgRNA comprises an editing template comprising a sequence selected from the group consisting of SEQ ID NOs. 16919-16926 of Table 3C. In some embodiments, the prime editing composition further comprises a ngRNA that comprises a spacer comprising a sequence selected from the group consisting of SEQ Identifiers: D1448-D1461, D1473-D1478, D1516, D1517, and D1529 of Table 3D. In some embodiments, the prime editing composition further comprises a ngRNA that comprises a spacer comprising a sequence selected from the group consisting of SEQ ID NOs. 21081-21094, 21106-21111, 21149, 21150, and 21162 of Table 3D.

In some embodiments, the PEgRNA comprises a spacer comprising a sequence selected from the group consisting of SEQ Identifiers: A635-A636 of Table 3A. In some embodiments, the PEgRNA comprises a spacer comprising a sequence as set forth in SEQ Identifiers. 983-984 of Table 3A. In some embodiments, the PEgRNA comprises a PBS comprising a sequence selected from the group consisting of SEQ ID Nos: B2583-B2591 of Table 3B. In some embodiments, the PEgRNA comprises a PBS comprising a sequence selected from the group consisting of SEQ ID NOs. 3222-3227 of Table 3B. In some embodiments, the PEgRNA comprises a PBS comprising a sequence selected from the group consisting of sequences AGTTCATT and AGTTCATTG as disclosed in Table 3B. In some embodiments, the PEgRNA comprises an editing template comprising a sequence selected from the group consisting of SEQ Identifiers: C12901-C12931 of Table 3C. In some embodiments, the PEgRNA comprises an editing template comprising a sequence selected from the group consisting of SEQ ID NOs. 16927-16957 of Table 3C. In some embodiments, the prime editing composition further comprises a ngRNA that comprises a spacer comprising a sequence selected from the group consisting of SEQ Identifiers: D1444-D1461, D1463-D1474, D1545, and D1546 of Table 3D. In some embodiments, the prime editing composition further comprises a ngRNA that comprises a spacer comprising a sequence selected from the group consisting of SEQ ID NOs. 21077-21094, 21096-21107, 21178, and 21179 of Table 3D.

In some embodiments, the PEgRNA comprises a spacer comprising a sequence selected from the group consisting of SEQ Identifiers: A637-A638 of Table 3A. In some embodiments, the PEgRNA comprises a spacer comprising a sequence as set forth in SEQ ID NOs. 985-986 of Table 3A. In some embodiments, the PEgRNA comprises a PBS comprising a sequence selected from the group consisting of SEQ Identifiers: B2592-B2600 of Table 3B. In some embodiments, the PEgRNA comprises a PBS comprising a sequence selected from the group consisting of SEQ ID NOs. 3229-3235 of Table 3B. In some embodiments, the PEgRNA comprises a PBS comprising a sequence selected from the group consisting of sequences CTTTAAGT and CTTTAAGTT as disclosed in Table 3B. In some embodiments, the PEgRNA comprises an editing template comprising a sequence selected from the group consisting of SEQ Identifiers: C12932-C12993 of Table 3C. In some embodiments, the PEgRNA comprises an editing template comprising a sequence selected from the group consisting of SEQ ID NOs. 16958-17019 of Table 3C. In some embodiments, the prime editing composition further comprises a ngRNA that comprises a spacer comprising a sequence selected from the group consisting of SEQ Identifiers: D1444-D1461, D1473, D1474, D1511, D1512, D1513, D1546, and D1547 of Table 3D. In some embodiments, the prime editing composition further comprises a ngRNA that comprises a spacer comprising a sequence selected from the group consisting of SEQ ID NOs. 21077-21094, 21106, 21107, 21144, 21145, 21146, 21179, and 21180 of Table 3D.

In some embodiments, the PEgRNA comprises a spacer comprising the sequence of SEQ Identifier: A669 of Table 3A. In some embodiments, the PEgRNA comprises a spacer comprising a sequence as set forth in SEQ ID NO. 1017 of Table 3A. In some embodiments, the PEgRNA comprises a PBS comprising a sequence selected from the group consisting of SEQ Identifiers: B2601-B2609 of Table 3B. In some embodiments, the PEgRNA comprises a PBS comprising a sequence selected from the group consisting of SEQ ID NOs. 3236-3242 of Table 3B. In some embodiments, the PEgRNA comprises a PBS comprising a sequence selected from the group consisting of sequences TCTTTAAG and TCTTTAAGT as disclosed in Table 3B. In some embodiments, the PEgRNA comprises an editing template comprising a sequence selected from the group consisting of SEQ Identifiers: C12994-C13024 of Table 3C. In some embodiments, the PEgRNA comprises an editing template comprising a sequence selected from the group consisting of SEQ ID NOs. 17020-17050 of Table 3C. In some embodiments, the prime editing composition further comprises a ngRNA that comprises a spacer comprising a sequence selected from the group consisting of SEQ Identifiers: D1444-D1461, D1473-D1474, D1546, and D1547 of Table 3D. In some embodiments, the prime editing composition further comprises a ngRNA that comprises a spacer comprising a sequence selected from the group consisting of SEQ ID NOs. 21077-21094, 21106-21107, 21179, and 21180 of Table 3D.

In some embodiments, the PEgRNA comprises a spacer comprising a sequence selected from the group consisting of SEQ Identifiers: A640-A641 of Table 3A. In some embodiments, the PEgRNA comprises a spacer comprising a sequence as set forth in SEQ ID NOs. 988-989 of Table 3A. In some embodiments, the PEgRNA comprises a PBS comprising a sequence selected from the group consisting of SEQ Identifiers: B2610-B2618 of Table 3B. In some embodiments, the PEgRNA comprises a PBS comprising a sequence selected from the group consisting of SEQ ID NOs. 3243-3249 of Table 3B. In some embodiments, the PEgRNA comprises a PBS comprising a sequence selected from the group consisting of sequences CTGAAATA and CTGAAATAA as disclosed in Table 3B. In some embodiments, the PEgRNA comprises an editing template comprising a sequence selected from the group consisting of SEQ Identifiers: C13025-C13047 of Table 3C. In some embodiments, the PEgRNA comprises an editing template comprising a sequence selected from the group consisting of SEQ ID NOs. 17051-17073 of Table 3C. In some embodiments, the prime editing composition further comprises a ngRNA that comprises a spacer comprising a sequence selected from the group consisting of SEQ Identifiers: D1489-D1507 of Table 3D. In some embodiments, the prime editing composition further comprises a ngRNA that comprises a spacer comprising a sequence selected from the group consisting of SEQ ID NOs. 21122-21140 of Table 3D.

In some embodiments, the PEgRNA comprises a spacer comprising the of SEQ Identifier: A642 of Table 3A. In some embodiments, the PEgRNA comprises a spacer comprising a sequence as set forth in SEQ ID NO. 990 of Table 3A. In some embodiments, the PEgRNA comprises a PBS comprising a sequence selected from the group consisting of SEQ Identifiers: B2619-B2627 of Table 3B. In some embodiments, the PEgRNA comprises a PBS comprising a sequence selected from the group consisting of SEQ ID NOs. 3250-3256 of Table 3B. In some embodiments, the PEgRNA comprises a PBS comprising a sequence selected from the group consisting of sequences GAAATAAA and GAAATAAAA as disclosed in Table 3B. In some embodiments, the PEgRNA comprises an editing template comprising a sequence selected from the group consisting of SEQ Identifiers: C13048-C13068 of Table 3C. In some embodiments, the PEgRNA comprises an editing template comprising a sequence selected from the group consisting of SEQ ID NOs. 17074-17094 of Table 3C. In some embodiments, the prime editing composition further comprises a ngRNA that comprises a spacer comprising a sequence selected from the group consisting of SEQ Identifiers: D1487-D1504 of Table 3D. In some embodiments, the prime editing composition further comprises a ngRNA that comprises a spacer comprising a sequence selected from the group consisting of SEQ ID NOs. 21120-21137 of Table 3D.

In some embodiments, the PEgRNA comprises a spacer comprising a sequence selected from the group consisting of SEQ Identifiers: A652-A653 of Table 3A. In some embodiments, the PEgRNA comprises a spacer comprising a sequence as set forth in SEQ ID NOs. 1000-1001 of Table 3A. In some embodiments, the PEgRNA comprises a PBS comprising a sequence selected from the group consisting of SEQ Identifiers: B2673-B2681 of Table 3B. In some embodiments, the PEgRNA comprises a PBS comprising a sequence selected from the group consisting of SEQ ID NOs. 3292-3298 of Table 3B. In some embodiments, the PEgRNA comprises a PBS comprising a sequence selected from the group consisting of sequences GATCGCAT and GATCGCATC as disclosed in Table 3B. In some embodiments, the PEgRNA comprises an editing template comprising a sequence selected from the group consisting of SEQ Identifiers: C13069-C13130 of Table 3C. In some embodiments, the PEgRNA comprises an editing template comprising a sequence selected from the group consisting of SEQ ID NOs. 17095-17156 of Table 3C. In some embodiments, the prime editing composition further comprises a ngRNA that comprises a spacer comprising a sequence selected from the group consisting of SEQ Identifiers: D1435-D1440 of Table 3D. In some embodiments, the prime editing composition further comprises a ngRNA that comprises a spacer comprising a sequence selected from the group consisting of SEQ ID NOs. 21068-21073 of Table 3D.

In some embodiments, the PEgRNA comprises a spacer comprising the sequence of SEQ Identifier: A654 of Table 3A. In some embodiments, the PEgRNA comprises a spacer comprising a sequence as set forth in SEQ ID NO. 1002 of Table 3A. In some embodiments, the PEgRNA comprises a PBS comprising a sequence selected from the group consisting of SEQ Identifiers: B2682-B2690 of Table 3B. In some embodiments, the PEgRNA comprises a PBS comprising a sequence selected from the group consisting of SEQ ID NOs. 3299-3305 of Table 3B. In some embodiments, the PEgRNA comprises a PBS comprising a sequence selected from the group consisting of sequences AGATCGCA and AGATCGCAT as disclosed in Table 3B. In some embodiments, the PEgRNA comprises an editing template comprising a sequence selected from the group consisting of SEQ Identifiers: C13131-C13161 of Table 3C. In some embodiments, the PEgRNA comprises an editing template comprising a sequence selected from the group consisting of SEQ ID NOs. 17157-17187 of Table 3C. In some embodiments, the prime editing composition further comprises a ngRNA that comprises a spacer comprising a sequence selected from the group consisting of SEQ Identifiers: D1491-D1504, D1542, and D1543 of Table 3D. In some embodiments, the prime editing composition further comprises a ngRNA that comprises a spacer comprising a sequence selected from the group consisting of SEQ ID NOs. 21124-21137, 21175, and 21176 of Table 3D.

In some embodiments, the PEgRNA comprises a spacer comprising a sequence selected from the group consisting of SEQ Identifiers: A643-A644 of Table 3A. In some embodiments, the PEgRNA comprises a spacer comprising a sequence as set forth in SEQ ID NOs. 991-992 of Table 3A. In some embodiments, the PEgRNA comprises a PBS comprising a sequence selected from the group consisting of SEQ Identifiers: B2628-B2636 of Table 3B. In some embodiments, the PEgRNA comprises a PBS comprising a sequence selected from the group consisting of SEQ ID NOs. 3257-3263 of Table 3B. In some embodiments, the PEgRNA comprises a PBS comprising a sequence selected from the group consisting of sequences GTTCATTG and GTTCATTGA as disclosed in Table 3B. In some embodiments, the PEgRNA comprises an editing template comprising a sequence selected from the group consisting of SEQ Identifiers: C13162-C13221 of Table 3C. In some embodiments, the PEgRNA comprises an editing template comprising a sequence selected from the group consisting of SEQ ID NOs. 17188-17247 of Table 3C. In some embodiments, the prime editing composition further comprises a ngRNA that comprises a spacer comprising a sequence selected from the group consisting of SEQ Identifiers: D1416-D1423, D1426-D1433, D1514, and D1515 of Table 3D. In some embodiments, the prime editing composition further comprises a ngRNA that comprises a spacer comprising a sequence selected from the group consisting of SEQ ID NOs. 21049-21056, 21059-21066, 21147, and 21148 of Table 3D.

In some embodiments, the PEgRNA comprises a spacer comprising a sequence selected from the group consisting of SEQ Identifiers: A657-A658 of Table 3A. In some embodiments, the PEgRNA comprises a spacer comprising a sequence as set forth in SEQ ID NOs. 1005-1006 of Table 3A. In some embodiments, the PEgRNA comprises a PBS comprising a sequence selected from the group consisting of SEQ Identifiers: B2700-B2708 of Table 3B. In some embodiments, the PEgRNA comprises a PBS comprising a sequence selected from the group consisting of SEQ ID NOs. 3313-3319 of Table 3B. In some embodiments, the PEgRNA comprises a PBS comprising a sequence selected from the group consisting of sequences GAAGGTAA and GAAGGTAAA as disclosed in Table 3B. In some embodiments, the PEgRNA comprises an editing template comprising a sequence selected from the group consisting of SEQ Identifiers: C13222-C13232 of Table 3C. In some embodiments, the PEgRNA comprises an editing template comprising a sequence selected from the group consisting of SEQ ID NOs: 17248-17258 of Table 3C. In some embodiments, the prime editing composition further comprises a ngRNA that comprises a spacer comprising a sequence selected from the group consisting of SEQ Identifiers: D1448-D1461, D1474-D1478, D1512, D1513, D1516, D1517, D1529, and D1536-D1539 of Table 3D. In some embodiments, the prime editing composition further comprises a ngRNA that comprises a spacer comprising a sequence selected from the group consisting of SEQ ID NOs. 21081-21094, 21107-21111, 21145, 21146, 21149, 21150, 21162, and 21169-21172 of Table 3D.

In some embodiments, the PEgRNA comprises a spacer comprising the sequence of SEQ Identifier A659 of Table 3A. In some embodiments, the PEgRNA comprises a spacer comprising a sequence as set forth in SEQ ID NO. 1007 of Table 3A. In some embodiments, the PEgRNA comprises a PBS comprising a sequence selected from the group consisting of SEQ Identifiers: B2709-B2717 of Table 3B. In some embodiments, the PEgRNA comprises a PBS comprising a sequence selected from the group consisting of SEQ ID NOs. 3320-3326 of Table 3B. In some embodiments, the PEgRNA comprises a PBS comprising a sequence selected from the group consisting of sequences AGAAGGTA and AGAAGGTAA as disclosed in Table 3B. In some embodiments, the PEgRNA comprises an editing template comprising a sequence selected from the group consisting of SEQ Identifiers: C13233-C13244 of Table 3C. In some embodiments, the PEgRNA comprises an editing template comprising a sequence selected from the group consisting of SEQ ID NOs. 17259-17270 of Table 3C. In some embodiments, the prime editing composition further comprises a ngRNA that comprises a spacer comprising a sequence selected from the group consisting of SEQ Identifiers: D1447-D1461, D1473-D1478, D1516, D1517, and D1529 of Table 3D. In some embodiments, the prime editing composition further comprises a ngRNA that comprises a spacer comprising a sequence selected from the group consisting of SEQ ID NOs. 21080-21094, 21106-21111, 21149, 21150, and 21162 of Table 3D.

In some embodiments, the PEgRNA comprises a spacer comprising a sequence selected from the group consisting of SEQ Identifiers: A626, A627, A628, and A670 of Table 3A. In some embodiments, the PEgRNA comprises a spacer comprising a sequence as set forth in SEQ ID NOs. 974-976 and 1018 of Table 3A. In some embodiments, the PEgRNA comprises a PBS comprising a sequence selected from the group consisting of SEQ Identifiers: B2538-B2546 of Table 3B. In some embodiments, the PEgRNA comprises a PBS comprising a sequence selected from the group consisting of SEQ ID NOs. 3187-3193 of Table 3B. In some embodiments, the PEgRNA comprises a PBS comprising a sequence selected from the group consisting of sequences TTTAAGTT and TTTAAGTTC as disclosed in Table 3B. In some embodiments, the PEgRNA comprises an editing template comprising a sequence selected from the group consisting of SEQ Identifiers: C13245-C13275 of Table 3C. In some embodiments, the PEgRNA comprises an editing template comprising a sequence selected from the group consisting of SEQ ID NOs. 17271-17301 of Table 3C. In some embodiments, the prime editing composition further comprises a ngRNA that comprises a spacer comprising a sequence selected from the group consisting of SEQ Identifiers: D1416-D1423, D1426-D1433, and D1479-D1485 of Table 3D. In some embodiments, the prime editing composition further comprises a ngRNA that comprises a spacer comprising a sequence selected from the group consisting of SEQ ID NOs. 21049-21056, 21059-21066, and 21112-21118 of Table 3D.

In some embodiments, the PEgRNA comprises a spacer comprising a sequence selected from the group consisting of SEQ Identifiers: A660-A661 of Table 3A. In some embodiments, the PEgRNA comprises a spacer comprising a sequence as set forth in SEQ ID NOs. 1008-1009 of Table 3A. In some embodiments, the PEgRNA comprises a PBS comprising a sequence selected from the group consisting of SEQ Identifiers: B2718-B2726 of Table 3B. In some embodiments, the PEgRNA comprises a PBS comprising a sequence selected from the group consisting of SEQ ID NOs. 3327-3333 of Table 3B. In some embodiments, the PEgRNA comprises a PBS comprising a sequence selected from the group consisting of sequences ATCGCATC and ATCGCATCT as disclosed in Table 3B. In some embodiments, the PEgRNA comprises an editing template comprising a sequence selected from the group consisting of SEQ Identifiers: C13276-C13305 of Table 3C. In some embodiments, the PEgRNA comprises an editing template comprising a sequence selected from the group consisting of SEQ ID NOs: 17302-17331 of Table 3C. In some embodiments, the prime editing composition further comprises a ngRNA that comprises a spacer comprising a sequence selected from the group consisting of SEQ Identifiers: D1491-D1507, D1542, and D1548 of Table 3D. In some embodiments, the prime editing composition further comprises a ngRNA that comprises a spacer comprising a sequence selected from the group consisting of SEQ ID NOs. 21124-21140, 21175, and 21181 of Table 3D.

In some embodiments, the PEgRNA comprises a spacer comprising the sequence of SEQ Identifier: A665 of Table 3A. In some embodiments, the PEgRNA comprises a spacer comprising a sequence as set forth in SEQ ID NO. 1013 of Table 3A. In some embodiments, the PEgRNA comprises a PBS comprising a sequence selected from the group consisting of SEQ Identifiers: B2736-B2744 of Table 3B. In some embodiments, the PEgRNA comprises a PBS comprising a sequence selected from the group consisting of SEQ ID NOs. 3341-3344 of Table 3B. In some embodiments, the PEgRNA comprises a PBS comprising a sequence selected from the group consisting of sequences CGCATCTG and CGCATCTGA as disclosed in Table 3B. In some embodiments, the PEgRNA comprises an editing template comprising a sequence selected from the group consisting of SEQ Identifiers: C13306-C13333 of Table 3C. In some embodiments, the PEgRNA comprises an editing template comprising a sequence selected from the group consisting of SEQ ID NOs: 17332-17359 of Table 3C. In some embodiments, the prime editing composition further comprises a ngRNA that comprises a spacer comprising a sequence selected from the group consisting of SEQ Identifiers: D1491-D1504 and D1542 of Table 3D. In some embodiments, the prime editing composition further comprises a ngRNA that comprises a spacer comprising a sequence selected from the group consisting of SEQ ID NOs. 21124-21137 and 21175 of Table 3D.

In some embodiments, the editing target sequence comprises a mutation as compared to a wild type CFTR gene.

In some embodiments, the editing target sequence or the complementary sequence on the CFTR gene thereof comprises a mutation that encodes an amino acid substitution K1177fs relative to a wild type CFTR polypeptide set forth in SEQ ID NO: 19, and wherein the editing template encodes an intended nucleotide edit that corrects the mutation. In some embodiments, the editing target sequence or the complementary sequence on the CFTR gene thereof comprises a C deletion mutation (c.3528delC) at position 117,627,581 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation.

In some embodiments, the editing target sequence or the complementary sequence on the CFTR gene thereof comprises a mutation that encodes an amino acid substitution relative to a wild type CFTR polypeptide set forth in SEQ ID NO: 19, and wherein the editing template encodes an intended nucleotide edit that corrects the mutation. In some embodiments, the editing target sequence or the complementary sequence on the CFTR gene thereof comprises a A-*T mutation (c.3529A>T) at position 117,627,582, a deletion of A (c.3530delA) at position 117,627,583, a duplication of a TCAA (c.3532_3535dupTCAA) at position 117,627,588, and/or a deletion of CCAA (c.3536_3539delCCAA) at position 117,627,589, of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide. In some embodiments, the editing template comprises a wild type CFTR gene sequence. Accordingly, in some embodiments, the newly synthesized single stranded DNA encoded by the editing template is capable of correcting the c.3528delC mutation, the c.3529A>T mutation, the c.3530delA mutation, the c.3532_3535dupTCAA mutation, and/or the c.3536_3539delCCAA mutation in the target CFTR gene. In some embodiments, the newly synthesized single stranded DNA encoded by the editing template is capable of correcting all of the c.3528delC mutation, the c.3529A>T mutation, the c.3530delA mutation, the c.3532_3535dupTCAA mutation, and the c.3536_3539delCCAA mutation in the target CFTR gene. In some embodiments, two or more of the c.3528delC mutation, the c.3529A>T mutation, the c.3530delA mutation, the c.3532_3535dupTCAA mutation, and the c.3536_3539delCCAA mutation are located on the same CFTR gene. In some embodiments, the newly synthesized single stranded DNA encoded by the editing template is capable of correcting the c.3528delC mutation, the c.3529A>T mutation, the c.3530delA mutation, the c.3532_3535dupTCAA mutation, and the c.3536_3539delCCAA mutation that are located on different CFTR genes. For example, in some embodiments, a subject may comprise two copies of CFTR genes, each comprising a different mutation selected from the group consisting of the c.3528delC mutation, the c.3529A>T mutation, the c.3530delA mutation, the c.3532_3535dupTCAA mutation, and the c.3536_3539delCCAA mutation, all of which may be corrected by an editing template of a single PEgRNA. In some embodiments, the editing template is capable of correcting one or more, or all of the c.3528delC mutation, the c.3529A>T mutation, the c.3530delA mutation, the c.3532_3535dupTCAA mutation, and the c.3536_3539delCCAA mutation that are in different subjects, wherein each subject may have one or more of the mutations.

In some embodiments, the PEgRNA comprises a spacer comprising a sequence selected from the group consisting of SEQ Identifiers: A671-A672 of Table 3A. In some embodiments, the PEgRNA comprises a spacer comprising a sequence as set forth in SEQ ID NOs. 1019-1020 of Table 3A. In some embodiments, the PEgRNA comprises a PBS comprising a sequence selected from the group consisting of SEQ Identifiers: B2745-B2753 of Table 3B. In some embodiments, the PEgRNA comprises a PBS comprising a sequence selected from the group consisting of SEQ ID NOs. 3348-3354 of Table 3B. In some embodiments, the PEgRNA comprises a PBS comprising a sequence selected from the group consisting of sequences GCATGTCA and GCATGTCAA as disclosed in Table 3B. In some embodiments, the PEgRNA comprises an editing template comprising a sequence selected from the group consisting of SEQ Identifiers: C13334-C13373 of Table 3C. In some embodiments, the PEgRNA comprises an editing template comprising a sequence selected from the group consisting of SEQ ID NOs: 17360-17399 of Table 3C. In some embodiments, the prime editing composition further comprises a ngRNA that comprises a spacer comprising a sequence selected from the group consisting of SEQ Identifiers: D1435-D1440, and D1549-D1553 of Table 3D. In some embodiments, the prime editing composition further comprises a ngRNA that comprises a spacer comprising a sequence selected from the group consisting of SEQ ID NOs. 21068-21073, and 21182-21186 of Table 3D.

In some embodiments, the PEgRNA comprises a spacer comprising a sequence selected from the group consisting of SEQ Identifiers: A673-A674 of Table 3A. In some embodiments, the PEgRNA comprises a spacer comprising a sequence as set forth in SEQ ID NOs. 1021-1022 of Table 3A. In some embodiments, the PEgRNA comprises a PBS comprising a sequence selected from the group consisting of SEQ Identifiers: B2754-B2762 of Table 3B. In some embodiments, the PEgRNA comprises a PBS comprising a sequence selected from the group consisting of SEQ ID NOs. 3355-3361 of Table 3B. In some embodiments, the PEgRNA comprises a PBS comprising a sequence selected from the group consisting of sequences CTCGAAAG and CTCGAAAGT as disclosed in Table 3B. In some embodiments, the PEgRNA comprises an editing template comprising a sequence selected from the group consisting of SEQ Identifiers: C13374-C13387 of Table 3C. In some embodiments, the PEgRNA comprises an editing template comprising a sequence selected from the group consisting of SEQ ID NOs: 17400-17413 of Table 3C. In some embodiments, the prime editing composition further comprises a ngRNA that comprises a spacer comprising a sequence selected from the group consisting of SEQ Identifiers: D1453-D1461, D1473-D1478, D1513, D1516, D1517, D1529, and D1536-D1539, D1554-D1563 of Table 3D. In some embodiments, the prime editing composition further comprises a ngRNA that comprises a spacer comprising a sequence selected from the group consisting of SEQ ID NOs. 21086-21094, 21106-21111, 21146, 21149, 21150, 21162, and 21169-21172 of Table 3D.

In some embodiments, the PEgRNA comprises a spacer comprising the sequence of SEQ ID Identifier: A675 of Table 3A. In some embodiments, the PEgRNA comprises a spacer comprising a sequence as set forth in SEQ ID NO. 1023 of Table 3A. In some embodiments, the PEgRNA comprises a PBS comprising a sequence selected from the group consisting of SEQ Identifiers: B2763-B2771 of Table 3B. In some embodiments, the PEgRNA comprises a PBS comprising a sequence selected from the group consisting of SEQ ID NOs. 3362-3368 of Table 3B. In some embodiments, the PEgRNA comprises a PBS comprising a sequence selected from the group consisting of sequences TCTCGAAA and TCTCGAAAG as disclosed in Table 3B. In some embodiments, the PEgRNA comprises an editing template comprising a sequence selected from the group consisting of SEQ Identifiers: C13388-C13395 of Table 3C. In some embodiments, the PEgRNA comprises an editing template comprising a sequence selected from the group consisting of SEQ ID NOs: 17414-17421 of Table 3C. In some embodiments, the prime editing composition further comprises a ngRNA that comprises a spacer comprising a sequence selected from the group consisting of SEQ Identifiers: D1453-D1461, D1473-D1478, D1516, D1517, D1529, D1556, and D1557-D1563 of Table 3D. In some embodiments, the prime editing composition further comprises a ngRNA that comprises a spacer comprising a sequence selected from the group consisting of SEQ ID NOs. 21086-21094, 21106-21111, 21149, 21150, 21162, 21189, and 21190-21196 of Table 3D.

In some embodiments, the PEgRNA comprises a spacer comprising a sequence selected from the group consisting of SEQ Identifiers: A676-A677 of Table 3A. In some embodiments, the PEgRNA comprises a spacer comprising a sequence as set forth in SEQ ID NOs. 1024-1025 of Table 3A. In some embodiments, the PEgRNA comprises a PBS comprising a sequence selected from the group consisting of SEQ Identifiers: B2772-B2780 of Table 3B. In some embodiments, the PEgRNA comprises a PBS comprising a sequence selected from the group consisting of SEQ ID NOs. 3369-3375 of Table 3B. In some embodiments, the PEgRNA comprises a PBS comprising a sequence selected from the group consisting of sequences AAGAATGG and AAGAATGGC as disclosed in Table 3B. In some embodiments, the PEgRNA comprises an editing template comprising a sequence selected from the group consisting of SEQ Identifiers: C13396-C13416 of Table 3C. In some embodiments, the PEgRNA comprises an editing template comprising a sequence selected from the group consisting of SEQ ID NOs: 17422-17442 of Table 3C. In some embodiments, the prime editing composition further comprises a ngRNA that comprises a spacer comprising a sequence selected from the group consisting of SEQ Identifiers: D1450-D1461, D1473-D1478, D1512, D1513, D1516, D1517, D1529, D1536, D1537, D1538, D1539, and D1554-D1562 of Table 3D. In some embodiments, the prime editing composition further comprises a ngRNA that comprises a spacer comprising a sequence selected from the group consisting of SEQ ID NOs. 21083-21094, 21106-21111, 21145, 21146, 21149, 21150, 21162, 21169, 21170, 21171, 21172, and 21187-21195 of Table 3D.

In some embodiments, the PEgRNA comprises a spacer comprising the sequence of SEQ Identifier: A678 of Table 3A. In some embodiments, the PEgRNA comprises a spacer comprising a sequence as set forth in SEQ ID NO. 1026 of Table 3A. In some embodiments, the PEgRNA comprises a PBS comprising a sequence selected from the group consisting of SEQ Identifiers: B2781-B2789 of Table 3B. In some embodiments, the PEgRNA comprises a PBS comprising a sequence selected from the group consisting of SEQ ID NOs. 3376-3382 of Table 3B. In some embodiments, the PEgRNA comprises a PBS comprising a sequence selected from the group consisting of sequences CAAGAATG and CAAGAATGG as disclosed in Table 3B. In some embodiments, the PEgRNA comprises an editing template comprising a sequence selected from the group consisting of SEQ Identifiers: C13417-C13438 of Table 3C. In some embodiments, the PEgRNA comprises an editing template comprising a sequence selected from the group consisting of SEQ ID NOs: 17443-17464 of Table 3C. In some embodiments, the prime editing composition further comprises a ngRNA that comprises a spacer comprising a sequence selected from the group consisting of SEQ Identifiers: D1450-D1461, D1473-D1478, D1516, D1517, D1529, D1556, D1557, D1558, D1559, D1560, D1561, and D1562 of Table 3D. In some embodiments, the prime editing composition further comprises a ngRNA that comprises a spacer comprising a sequence selected from the group consisting of SEQ ID NOs. 21083-21094, 21106-21111, 21149, 21150, 21162, 21189, 21190, 21191, 21192, 21193, 21194, and 21195 of Table 3D.

In some embodiments, the PEgRNA comprises a spacer comprising the sequence of SEQ Identifier: A679 of Table 3A. In some embodiments, the PEgRNA comprises a spacer comprising a sequence as set forth in SEQ ID NO. 1027 of Table 3A. In some embodiments, the PEgRNA comprises a PBS comprising a sequence selected from the group consisting of SEQ Identifiers: B2790-B2798 of Table 3B. In some embodiments, the PEgRNA comprises a PBS comprising a sequence selected from the group consisting of SEQ ID NOs. 3383-3389 of Table 3B. In some embodiments, the PEgRNA comprises a PBS comprising a sequence selected from the group consisting of sequences CAATGAAC and CAATGAACT as disclosed in Table 3B. In some embodiments, the PEgRNA comprises an editing template comprising a sequence selected from the group consisting of SEQ Identifiers: C13439-C13452 of Table 3C. In some embodiments, the PEgRNA comprises an editing template comprising a sequence selected from the group consisting of SEQ ID NOs: 17465-17478 of Table 3C. In some embodiments, the prime editing composition further comprises a ngRNA that comprises a spacer comprising a sequence selected from the group consisting of SEQ Identifiers: D1496-D1504, and D1564-D1569 of Table 3D. In some embodiments, the prime editing composition further comprises a ngRNA that comprises a spacer comprising a sequence selected from the group consisting of SEQ ID NOs. 21129-21137, and 21197-21202 of Table 3D.

In some embodiments, the PEgRNA comprises a spacer comprising a sequence selected from the group consisting of SEQ Identifier: A680-A681 of Table 3A. In some embodiments, the PEgRNA comprises a spacer comprising a sequence as set forth in SEQ ID NOs. 1028-1029 of Table 3A. In some embodiments, the PEgRNA comprises a PBS comprising a sequence selected from the group consisting of SEQ Identifiers: B2799-B2807 of Table 3B. In some embodiments, the PEgRNA comprises a PBS comprising a sequence selected from the group consisting of SEQ ID NOs. 3390-3396 of Table 3B. In some embodiments, the PEgRNA comprises a PBS comprising a sequence selected from the group consisting of sequences GGCATGTC and GGCATGTCA as disclosed in Table 3B. In some embodiments, the PEgRNA comprises an editing template comprising a sequence selected from the group consisting of SEQ Identifiers: C13453-C13473 of Table 3C. In some embodiments, the PEgRNA comprises an editing template comprising a sequence selected from the group consisting of SEQ ID NOs: 17479-17499 of Table 3C. In some embodiments, the prime editing composition further comprises a ngRNA that comprises a spacer comprising a sequence selected from the group consisting of SEQ Identifiers: D1496-D1507, and D1564-D1575 of Table 3D. In some embodiments, the prime editing composition further comprises a ngRNA that comprises a spacer comprising a sequence selected from the group consisting of SEQ ID NOs. 21129-21140, and 21197-21208 of Table 3D.

In some embodiments, the PEgRNA comprises a spacer comprising a sequence selected from the group consisting of SEQ Identifier: A682-A684 of Table 3A. In some embodiments, the PEgRNA comprises a spacer comprising a sequence as set forth in SEQ ID NOs. 1030-1032 of Table 3A. In some embodiments, the PEgRNA comprises a PBS comprising a sequence selected from the group consisting of SEQ Identifiers: B2808-B2816 of Table 3B. In some embodiments, the PEgRNA comprises a PBS comprising a sequence selected from the group consisting of SEQ ID NOs. 3397-3403 of Table 3B. In some embodiments, the PEgRNA comprises a PBS comprising a sequence selected from the group consisting of sequences TCGAAAGT and TCGAAAGTT as disclosed in Table 3B. In some embodiments, the PEgRNA comprises an editing template comprising a sequence selected from the group consisting of SEQ Identifiers: C13474-C13479 of Table 3C. In some embodiments, the PEgRNA comprises an editing template comprising a sequence selected from the group consisting of SEQ ID NOs: 17500-17505 of Table 3C. In some embodiments, the prime editing composition further comprises a ngRNA that comprises a spacer comprising a sequence selected from the group consisting of SEQ Identifiers: D1475, D1476, D1477, D1480, D1483, D1486, D1515, D1521-D1528, and D1576-D1581 of Table 3D. In some embodiments, the prime editing composition further comprises a ngRNA that comprises a spacer comprising a sequence selected from the group consisting of SEQ ID NOs. 21108, 21109, 21110, 21113, 21116, 21119, 21148, 21154-21161, and 21209-21214 of Table 3D.

In some embodiments, the PEgRNA comprises a spacer comprising a sequence selected from the group consisting of SEQ Identifier: A685-A686 of Table 3A. In some embodiments, the PEgRNA comprises a spacer comprising a sequence as set forth in SEQ ID NOs. 1033-1034 of Table 3A. In some embodiments, the PEgRNA comprises a PBS comprising a sequence selected from the group consisting of SEQ Identifiers: B2817-B2825 of Table 3B. In some embodiments, the PEgRNA comprises a PBS comprising a sequence selected from the group consisting of SEQ ID NOs. 3404-3410 of Table 3B. In some embodiments, the PEgRNA comprises a PBS comprising a sequence selected from the group consisting of sequences GACTCGGC and GACTCGGCT as disclosed in Table 3B. In some embodiments, the PEgRNA comprises an editing template comprising a sequence selected from the group consisting of SEQ Identifiers: C13480-C13481 of Table 3C. In some embodiments, the PEgRNA comprises an editing template comprising a sequence selected from the group consisting of SEQ ID NOs: 17506-17507 of Table 3C. In some embodiments, the prime editing composition further comprises a ngRNA that comprises a spacer comprising a sequence selected from the group consisting of SEQ Identifiers: D1435-D1440, D1549, D1551, D1582, and D1583 of Table 3D. In some embodiments, the prime editing composition further comprises a ngRNA that comprises a spacer comprising a sequence selected from the group consisting of SEQ ID NOs 21068-21073, 21182, 21184, 21215, and 21216. of Table 3D.

In some embodiments, the PEgRNA comprises a spacer comprising the sequence of SEQ Identifier: A687 of Table 3A. In some embodiments, the PEgRNA comprises a spacer comprising a sequence as set forth in SEQ ID NO. 1035 of Table 3A. In some embodiments, the PEgRNA comprises a PBS comprising a sequence selected from the group consisting of SEQ Identifiers: B2826-B2834 of Table 3B. In some embodiments, the PEgRNA comprises a PBS comprising a sequence selected from the group consisting of SEQ ID NOs. 3411-3417 of Table 3B. In some embodiments, the PEgRNA comprises a PBS comprising a sequence selected from the group consisting of sequences AGACTCGG and AGACTCGGC as disclosed in Table 3B. In some embodiments, the PEgRNA comprises an editing template comprising a sequence selected from the group consisting of SEQ Identifiers: C13482-C13483 of Table 3C. In some embodiments, the PEgRNA comprises an editing template comprising a sequence selected from the group consisting of SEQ ID NOs: 17508-17509 of Table 3C. In some embodiments, the prime editing composition further comprises a ngRNA that comprises a spacer comprising a sequence selected from the group consisting of SEQ Identifiers: D1496-D1504, and D1564-D1568 of Table 3D. In some embodiments, the prime editing composition further comprises a ngRNA that comprises a spacer comprising a sequence selected from the group consisting of SEQ ID NOs. 21129-21137, and 21197-21201 of Table 3D.

In some embodiments, the PEgRNA comprises a spacer comprising a sequence selected from the group consisting of SEQ Identifiers: A688-A689 of Table 3A. In some embodiments, the PEgRNA comprises a spacer comprising a sequence as set forth in SEQ ID NOs. 1036-1037 of Table 3A. In some embodiments, the PEgRNA comprises a PBS comprising a sequence selected from the group consisting of SEQ Identifiers: B2835-B2843 of Table 3B. In some embodiments, the PEgRNA comprises a PBS comprising a sequence selected from the group consisting of SEQ ID NOs. 3418-3424 of Table 3B. In some embodiments, the PEgRNA comprises a PBS comprising a sequence selected from the group consisting of sequences GGTTTACC and GGTTTACCT as disclosed in Table 3B. In some embodiments, the PEgRNA comprises an editing template comprising a sequence selected from the group consisting of SEQ Identifiers: C13484-C13514 of Table 3C. In some embodiments, the PEgRNA comprises an editing template comprising a sequence selected from the group consisting of SEQ ID NOs: 17510-17540 of Table 3C. In some embodiments, the prime editing composition further comprises a ngRNA that comprises a spacer comprising a sequence selected from the group consisting of SEQ Identifiers: D1435-D1440, and D1549-D1553 of Table 3D. In some embodiments, the prime editing composition further comprises a ngRNA that comprises a spacer comprising a sequence selected from the group consisting of SEQ ID NOs. 21068-21073, and 21182-21186 of Table 3D.

In some embodiments, the PEgRNA comprises a spacer comprising the sequence of SEQ Identifier: A690 of Table 3A. In some embodiments, the PEgRNA comprises a spacer comprising a sequence as set forth in SEQ ID NO. 1038 of Table 3A. In some embodiments, the PEgRNA comprises a PBS comprising a sequence selected from the group consisting of SEQ Identifiers: B2844-B2852 of Table 3B. In some embodiments, the PEgRNA comprises a PBS comprising a sequence selected from the group consisting of SEQ ID NOs. 3425-3431 of Table 3B. In some embodiments, the PEgRNA comprises a PBS comprising a sequence selected from the group consisting of sequences AGGTTTAC and AGGTTTACC as disclosed in Table 3B. In some embodiments, the PEgRNA comprises an editing template comprising a sequence selected from the group consisting of SEQ Identifiers: C13515-C13545 of Table 3C. In some embodiments, the PEgRNA comprises an editing template comprising a sequence selected from the group consisting of SEQ ID NOs: 17541-17571 of Table 3C. In some embodiments, the prime editing composition further comprises a ngRNA that comprises a spacer comprising a sequence selected from the group consisting of SEQ Identifiers: D1497-D1504, D1564-D1571, D1584, D1585, and D1586 of Table 3D. In some embodiments, the prime editing composition further comprises a ngRNA that comprises a spacer comprising a sequence selected from the group consisting of SEQ ID NOs. 21130-21137, 21197-21204, 21217, 21218, and 21219 of Table 3D.

In some embodiments, the PEgRNA comprises a spacer comprising a sequence selected from the group consisting of SEQ Identifiers: A691-A692 of Table 3A. In some embodiments, the PEgRNA comprises a spacer comprising a sequence as set forth in SEQ ID NOs. 1039-1040 of Table 3A. In some embodiments, the PEgRNA comprises a PBS comprising a sequence selected from the group consisting of SEQ Identifiers: B2853-B2861 of Table 3B. In some embodiments, the PEgRNA comprises a PBS comprising a sequence selected from the group consisting of SEQ ID NOs. 3432-3438 of Table 3B. In some embodiments, the PEgRNA comprises a PBS comprising a sequence selected from the group consisting of sequences AAAGTTAT and AAAGTTATG as disclosed in Table 3B. In some embodiments, the PEgRNA comprises an editing template comprising a sequence selected from the group consisting of SEQ Identifiers: C13546-C13551 of Table 3C. In some embodiments, the PEgRNA comprises an editing template comprising a sequence selected from the group consisting of SEQ ID NOs: 17572-17577 of Table 3C. In some embodiments, the prime editing composition further comprises a ngRNA that comprises a spacer comprising a sequence selected from the group consisting of SEQ Identifiers: D1421, D1422, D1423, D1431, D1432, D1433, D1514, D1515, D1578, D1587, and D1588-D1595 of Table 3D. In some embodiments, the prime editing composition further comprises a ngRNA that comprises a spacer comprising a sequence selected from the group consisting of SEQ ID NOs. 21054, 21055, 21056, 21064, 21065, 21066, 21147, 21148, 21211, 21220, and 21221-21228 of Table 3D.

In some embodiments, the PEgRNA comprises a spacer comprising the sequence of SEQ Identifier: A693 of Table 3A. In some embodiments, the PEgRNA comprises a spacer comprising a sequence as set forth in SEQ ID NO. 1041 of Table 3A. In some embodiments, the PEgRNA comprises a PBS comprising a sequence selected from the group consisting of SEQ Identifiers: B2862-B2870 of Table 3B. In some embodiments, the PEgRNA comprises a PBS comprising a sequence selected from the group consisting of SEQ ID NOs. 3439-3445 of Table 3B. In some embodiments, the PEgRNA comprises a PBS comprising a sequence selected from the group consisting of sequences GAAAGTTA and GAAAGTTAT as disclosed in Table 3B. In some embodiments, the PEgRNA comprises an editing template comprising a sequence selected from the group consisting of SEQ Identifiers: C13552-C13555 of Table 3C. In some embodiments, the PEgRNA comprises an editing template comprising a sequence selected from the group consisting of SEQ ID NOs: 17578-17581 of Table 3C. In some embodiments, the prime editing composition further comprises a ngRNA that comprises a spacer comprising a sequence selected from the group consisting of SEQ Identifiers: D1454-D1461, D1473-D1478, D1516, D1517, D1529, and D1556-D1563 of Table 3D. In some embodiments, the prime editing composition further comprises a ngRNA that comprises a spacer comprising a sequence selected from the group consisting of SEQ ID NOs. 21087-21094, 21106-21111, 21149, 21150, 21162, and 21189-21196 of Table 3D.

In some embodiments, the PEgRNA comprises a spacer comprising a sequence selected from the group consisting of SEQ Identifiers: A694-A695 of Table 3A. In some embodiments, the PEgRNA comprises a spacer comprising a sequence as set forth in SEQ ID NOs. 1042-1043 of Table 3A. In some embodiments, the PEgRNA comprises a PBS comprising a sequence selected from the group consisting of SEQ Identifiers: B2871-B2879 of Table 3B. In some embodiments, the PEgRNA comprises a PBS comprising a sequence selected from the group consisting of SEQ ID NOs. 3446-3452 of Table 3B. In some embodiments, the PEgRNA comprises a PBS comprising a sequence selected from the group consisting of sequences AAACCATA and AAACCATAC as disclosed in Table 3B. In some embodiments, the PEgRNA comprises an editing template comprising a sequence selected from the group consisting of SEQ Identifiers: C13556-C13585 of Table 3C. In some embodiments, the PEgRNA comprises an editing template comprising a sequence selected from the group consisting of SEQ ID NOs: 17582-17611 of Table 3C. In some embodiments, the prime editing composition further comprises a ngRNA that comprises a spacer comprising a sequence selected from the group consisting of SEQ Identifiers: D1450-D1478, D1516, D1517, D1529, D1556, D1557, D1558, D1596, D1597, D1598, and D1599 of Table 3D. In some embodiments, the prime editing composition further comprises a ngRNA that comprises a spacer comprising a sequence selected from the group consisting of SEQ ID NOs. 21083-21111, 21149, 21150, 21162, 21189, 21190, 21191, 21229, 21230, 21231, and 21232 of Table 3D.

In some embodiments, the PEgRNA comprises a spacer comprising a sequence selected from the group consisting of SEQ Identifier: A696-A698 of Table 3A. In some embodiments, the PEgRNA comprises a spacer comprising a sequence as set forth in SEQ ID NOs. 1044-1046 of Table 3A. In some embodiments, the PEgRNA comprises a PBS comprising a sequence selected from the group consisting of SEQ Identifiers: B2880-B2888 of Table 3B. In some embodiments, the PEgRNA comprises a PBS comprising a sequence selected from the group consisting of SEQ ID NOs. 3453-3459 of Table 3B. In some embodiments, the PEgRNA comprises a PBS comprising a sequence selected from the group consisting of sequences AGAATGGC and AGAATGGCC as disclosed in Table 3B. In some embodiments, the PEgRNA comprises an editing template comprising a sequence selected from the group consisting of SEQ Identifiers: C13586-C13605 of Table 3C. In some embodiments, the PEgRNA comprises an editing template comprising a sequence selected from the group consisting of SEQ ID NOs: 17612-17631 of Table 3C. In some embodiments, the prime editing composition further comprises a ngRNA that comprises a spacer comprising a sequence selected from the group consisting of SEQ Identifiers: D1475, D1476, D1477, D1480, D1483, D1486, D1515, D1521, D1522-D1528, and D1576-D1581 of Table 3D. In some embodiments, the prime editing composition further comprises a ngRNA that comprises a spacer comprising a sequence selected from the group consisting of SEQ ID NOs. 21108, 21109, 21110, 21113, 21119, 21148, 21154, 21155-21161, and 21209-21214 of Table 3D.

In some embodiments, the PEgRNA comprises a spacer comprising a sequence selected from the group consisting of SEQ Identifiers: A699-A702 of Table 3A. In some embodiments, the PEgRNA comprises a spacer comprising a sequence as set forth in SEQ ID NOs. 1047-1050 of Table 3A. In some embodiments, the PEgRNA comprises a PBS comprising a sequence selected from the group consisting of SEQ Identifiers: B2889-B2897 of Table 3B. In some embodiments, the PEgRNA comprises a PBS comprising a sequence selected from the group consisting of SEQ ID NOs. 3460-3466 of Table 3B. In some embodiments, the PEgRNA comprises a PBS comprising a sequence selected from the group consisting of sequences AGTTATGA and AGTTATGAT as disclosed in Table 3B. In some embodiments, the PEgRNA comprises an editing template comprising a sequence selected from the group consisting of SEQ Identifiers: C13606-C13607 of Table 3C. In some embodiments, the PEgRNA comprises an editing template comprising a sequence selected from the group consisting of SEQ ID NOs: 17632-17633 of Table 3C. In some embodiments, the prime editing composition further comprises a ngRNA that comprises a spacer comprising a sequence selected from the group consisting of SEQ Identifiers: D1421, D1422, D1423, D1431, D1432, D1433, D1514, D1515, D1578, D1587-D1595, D1600, and D1601 of Table 3D. In some embodiments, the prime editing composition further comprises a ngRNA that comprises a spacer comprising a sequence selected from the group consisting of SEQ ID NOs. 21054, 21055, 21056, 21064, 21065, 21066, 21147, 21148, 21211, 21220-21228, 21233, and 21234 of Table 3D.

In some embodiments, the PEgRNA comprises a spacer comprising the sequence of SEQ Identifier: A703 of Table 3A. In some embodiments, the PEgRNA comprises a spacer comprising a sequence as set forth in SEQ ID NO. 1051 of Table 3A. In some embodiments, the PEgRNA comprises a PBS comprising a sequence selected from the group consisting of SEQ Identifiers: B2898-B2906 of Table 3B. In some embodiments, the PEgRNA comprises a PBS comprising a sequence selected from the group consisting of SEQ ID NOs. 3467-3473 of Table 3B. In some embodiments, the PEgRNA comprises a PBS comprising a sequence selected from the group consisting of sequences CAACCAAA and CAACCAAAC as disclosed in Table 3B. In some embodiments, the PEgRNA comprises an editing template comprising a sequence selected from the group consisting of SEQ Identifiers: C13608-C13638 of Table 3C. In some embodiments, the PEgRNA comprises an editing template comprising a sequence selected from the group consisting of SEQ ID NOs: 17634-17664 of Table 3C. In some embodiments, the prime editing composition further comprises a ngRNA that comprises a spacer comprising a sequence selected from the group consisting of SEQ Identifiers: D1450-D1461, D1473-D1478, D1516, D1517, D1529, D1556, D1557, and D1599 of Table 3D. In some embodiments, the prime editing composition further comprises a ngRNA that comprises a spacer comprising a sequence selected from the group consisting of SEQ ID NOs. 21083-21094, 21106-21111, 21149, 21150, 21162, 21189, 21190, and 21232 of Table 3D.

In some embodiments, the PEgRNA comprises a spacer comprising a sequence selected from the group consisting of SEQ Identifiers: A704-A705 of Table 3A. In some embodiments, the PEgRNA comprises a spacer comprising a sequence as set forth in SEQ ID NOs. 1052-1053 of Table 3A. In some embodiments, the PEgRNA comprises a PBS comprising a sequence selected from the group consisting of SEQ Identifiers: B2907-B2915 of Table 3B. In some embodiments, the PEgRNA comprises a PBS comprising a sequence selected from the group consisting of SEQ ID NOs. 3474-3480 of Table 3B. In some embodiments, the PEgRNA comprises a PBS comprising a sequence selected from the group consisting of sequences AACTTAAA and AACTTAAAG as disclosed in Table 3B. In some embodiments, the PEgRNA comprises an editing template comprising a sequence selected from the group consisting of SEQ Identifiers: C13639-C13647 of Table 3C. In some embodiments, the PEgRNA comprises an editing template comprising a sequence selected from the group consisting of SEQ ID NOs: 17665-17673 of Table 3C. In some embodiments, the prime editing composition further comprises a ngRNA that comprises a spacer comprising a sequence selected from the group consisting of SEQ Identifiers: D1496-D1507, D1564-D1569, and D1572-D1574. In some embodiments, the prime editing composition further comprises a ngRNA that comprises a spacer comprising a sequence selected from the group consisting of SEQ ID NOs. 21129-21140, 21197-21202, and 21205-21207 of Table 3D.

In some embodiments, the PEgRNA comprises a spacer comprising a sequence selected from the group consisting of SEQ Identifier: A706-A709 of Table 3A. In some embodiments, the PEgRNA comprises a spacer comprising a sequence as set forth in SEQ ID NOs. 1054-1057 of Table 3A. In some embodiments, the PEgRNA comprises a PBS comprising a sequence selected from the group consisting of SEQ Identifiers: B2916-B2924 of Table 3B. In some embodiments, the PEgRNA comprises a PBS comprising a sequence selected from the group consisting of SEQ ID NOs. 3481-3487 of Table 3B. In some embodiments, the PEgRNA comprises a PBS comprising a sequence selected from the group consisting of sequences GTCAACCA and GTCAACCAA as disclosed in Table 3B. In some embodiments, the PEgRNA comprises an editing template comprising a sequence selected from the group consisting of SEQ Identifiers: C13648-C13678 of Table 3C. In some embodiments, the PEgRNA comprises an editing template comprising a sequence selected from the group consisting of SEQ ID NOs: 17674-17704 of Table 3C. In some embodiments, the prime editing composition further comprises a ngRNA that comprises a spacer comprising a sequence selected from the group consisting of SEQ Identifiers: D1420, D1421, D1422, D1423, D1430, D1431, D1432, D1433, D1475, D1476, D1477, D1479, D1480, D1482, D1483, D1485, D1486, D1514, D1515, D1521, D1522, D1523, D1524, D1525, D1526, D1527, D1528, D1588, D1589, D1593, D1594, D1602, and D1603 of Table 3D. In some embodiments, the prime editing composition further comprises a ngRNA that comprises a spacer comprising a sequence selected from the group consisting of SEQ ID NOs. 21053-21056, 21063-21066, 21108-21110, 21112-21116, 21118, 21119, 21147, 21148, 21154-21161, 21221, 21222, 21235 and 21236 of Table 3D.

In some embodiments, the PEgRNA comprises a spacer comprising a sequence selected from the group consisting of SEQ Identifiers: A710-A712 of Table 3A. In some embodiments, the PEgRNA comprises a spacer comprising a sequence as set forth in SEQ ID NOs. 1058-1060 of Table 3A. In some embodiments, the PEgRNA comprises a PBS comprising a sequence selected from the group consisting of SEQ Identifiers: B2925-B2933 of Table 3B. In some embodiments, the PEgRNA comprises a PBS comprising a sequence selected from the group consisting of SEQ ID NOs. 3488-3494 of Table 3B. In some embodiments, the PEgRNA comprises a PBS comprising a sequence selected from the group consisting of sequences ATACAAGA and ATACAAGAA as disclosed in Table 3B. In some embodiments, the PEgRNA comprises an editing template comprising a sequence selected from the group consisting of SEQ Identifiers: C13679-C13703 of Table 3C. In some embodiments, the PEgRNA comprises an editing template comprising a sequence selected from the group consisting of SEQ ID NOs: 17705-17729 of Table 3C. In some embodiments, the prime editing composition further comprises a ngRNA that comprises a spacer comprising a sequence selected from the group consisting of SEQ Identifiers: D1475, D1476, D1477, D1479, D1480, D1482, D1483, D1485, D1486, D1515, D1521, D1522, D1523, D1524, D1525, D1526, D1527, D1528, D1576, D1577, D1578, D1579, D1580, and D1581 of Table 3D. In some embodiments, the prime editing composition further comprises a ngRNA that comprises a spacer comprising a sequence selected from the group consisting of SEQ ID NOs. 21108, 21109, 21110, 21112, 21113, 21115, 21116, 21118, 21119, 21148, 21154-21161, and 21209-21214 of Table 3D.

In some embodiments, the PEgRNA comprises a spacer comprising a sequence selected from the group consisting of SEQ Identifier: A713-A714 of Table 3A. In some embodiments, the PEgRNA comprises a spacer comprising a sequence as set forth in SEQ ID NOs. 1061-1062 of Table 3A. In some embodiments, the PEgRNA comprises a PBS comprising a sequence selected from the group consisting of SEQ Identifiers: B2934-B2942 of Table 3B. In some embodiments, the PEgRNA comprises a PBS comprising a sequence selected from the group consisting of SEQ ID NOs.=3495-3501 of Table 3B. In some embodiments, the PEgRNA comprises a PBS comprising a sequence selected from the group consisting of sequences AGTCAACC and AGTCAACCA as disclosed in Table 3B. In some embodiments, the PEgRNA comprises an editing template comprising a sequence selected from the group consisting of SEQ Identifiers: C13704-C13734 of Table 3C. In some embodiments, the PEgRNA comprises an editing template comprising a sequence selected from the group consisting of SEQ ID NOs: 17730-17760 of Table 3C. In some embodiments, the prime editing composition further comprises a ngRNA that comprises a spacer comprising a sequence selected from the group consisting of SEQ Identifiers: D1450-D1461, D1473-D1478, D1512, D1513, D1516, D1517, D1529, D1536, D1537, D1538, D1539, D1556, D1557, and D1599 of Table 3D. In some embodiments, the prime editing composition further comprises a ngRNA that comprises a spacer comprising a sequence selected from the group consisting of SEQ ID NOs. 21083-21094, 21106-21111, 21145, 21146, 21149, 21150, 21162, 21169, 21170, 21171, 21172, 21189, 21190, and 21232 of Table 3D.

In some embodiments, the PEgRNA comprises a spacer comprising the sequence of SEQ Identifier: A715 of Table 3A. In some embodiments, the PEgRNA comprises a spacer comprising a sequence as set forth in SEQ ID NO. 1063 of Table 3A. In some embodiments, the PEgRNA comprises a PBS comprising a sequence selected from the group consisting of SEQ Identifiers: B2943-B2951 of Table 3B. In some embodiments, the PEgRNA comprises a PBS comprising a sequence selected from the group consisting of SEQ ID NOs. 3502-3508 of Table 3B. In some embodiments, the PEgRNA comprises a PBS comprising a sequence selected from the group consisting of sequences AAGTCAAC and AAGTCAACC as disclosed in Table 3B. In some embodiments, the PEgRNA comprises an editing template comprising a sequence selected from the group consisting of SEQ Identifiers: C13735-C13765 of Table 3C. In some embodiments, the PEgRNA comprises an editing template comprising a sequence selected from the group consisting of SEQ ID NOs: 17761-17791 of Table 3C. In some embodiments, the prime editing composition further comprises a ngRNA that comprises a spacer comprising a sequence selected from the group consisting of SEQ Identifiers: D1450-D1461, D1473-D1478, D1516, D1517, D1529, D1556, D1557, and D1599 of Table 3D. In some embodiments, the prime editing composition further comprises a ngRNA that comprises a spacer comprising a sequence selected from the group consisting of SEQ ID NOs. 21083-21094, 21106-21111, 21149, 21150, 21162, 21189, 21190, and 21232 of Table 3D.

In some embodiments, the PEgRNA comprises a spacer comprising a sequence selected from the group consisting of SEQ Identifier: A716-A717 of Table 3A. In some embodiments, the PEgRNA comprises a spacer comprising a sequence as set forth in SEQ ID NOs. 1064-1065 of Table 3A. In some embodiments, the PEgRNA comprises a PBS comprising a sequence selected from the group consisting of SEQ Identifiers: B2952-B2960 of Table 3B. In some embodiments, the PEgRNA comprises a PBS comprising a sequence selected from the group consisting of SEQ ID NOs. 3509-3515 of Table 3B. In some embodiments, the PEgRNA comprises a PBS comprising a sequence selected from the group consisting of sequences AAGTTATG and AAGTTATGA as disclosed in Table 3B. In some embodiments, the PEgRNA comprises an editing template comprising a sequence selected from the group consisting of SEQ Identifiers: C13766-C13767 of Table 3C. In some embodiments, the PEgRNA comprises an editing template comprising a sequence selected from the group consisting of SEQ ID NOs: 17792-17793 of Table 3C. In some embodiments, the prime editing composition further comprises a ngRNA that comprises a spacer comprising a sequence selected from the group consisting of SEQ Identifiers: D1454-D1461, D1466-D1478, D1516, D1517, D1529, D1556-D1563, D1596, D1597, D1598, and D1604 of Table 3D. In some embodiments, the prime editing composition further comprises a ngRNA that comprises a spacer comprising a sequence selected from the group consisting of SEQ ID NOs. 21087-21094, 21099-21111, 21149, 21189-21196, 21229, 21230, 21231, and 21237 of Table 3D.

In some embodiments, the PEgRNA comprises a spacer comprising a sequence selected from the group consisting of SEQ Identifiers: A718-A719 of Table 3A. In some embodiments, the PEgRNA comprises a spacer comprising a sequence as set forth in SEQ ID NOs. 1066-1067 of Table 3A. In some embodiments, the PEgRNA comprises a PBS comprising a sequence selected from the group consisting of SEQ Identifiers: B2961-B2969 of Table 3B. In some embodiments, the PEgRNA comprises a PBS comprising a sequence selected from the group consisting of SEQ ID NOs. 3516-3522 of Table 3B. In some embodiments, the PEgRNA comprises a PBS comprising a sequence selected from the group consisting of sequences GTTGGCAT and GTTGGCATG as disclosed in Table 3B. In some embodiments, the PEgRNA comprises an editing template comprising a sequence selected from the group consisting of SEQ Identifiers: C13768-C13791 of Table 3C. In some embodiments, the PEgRNA comprises an editing template comprising a sequence selected from the group consisting of SEQ ID NOs: 17794-17817 of Table 3C. In some embodiments, the prime editing composition further comprises a ngRNA that comprises a spacer comprising a sequence selected from the group consisting of SEQ Identifiers: D1497-D1504, D1564-D1571, D1605, and D1606 of Table 3D. In some embodiments, the prime editing composition further comprises a ngRNA that comprises a spacer comprising a sequence selected from the group consisting of SEQ ID NOs. 21130-21137, 21197-21204, 21238, and 21239 of Table 3D.

In some embodiments, the PEgRNA comprises a spacer comprising the sequence of SEQ Identifier A720 of Table 3A. In some embodiments, the PEgRNA comprises a spacer comprising a sequence as set forth in SEQ ID NO. 1068 of Table 3A. In some embodiments, the PEgRNA comprises a PBS comprising a sequence selected from the group consisting of SEQ Identifiers: B2970-B2978 of Table 3B. In some embodiments, the PEgRNA comprises a PBS comprising a sequence selected from the group consisting of SEQ ID NOs. 3523-3529 of Table 3B. In some embodiments, the PEgRNA comprises a PBS comprising a sequence selected from the group consisting of sequences TGTTGGCA and TGTTGGCAT as disclosed in Table 3B. In some embodiments, the PEgRNA comprises an editing template comprising a sequence selected from the group consisting of SEQ Identifiers: C13792-C13816 of Table 3C. In some embodiments, the PEgRNA comprises an editing template comprising a sequence selected from the group consisting of SEQ ID NOs: 17818-17842 of Table 3C. In some embodiments, the prime editing composition further comprises a ngRNA that comprises a spacer comprising a sequence selected from the group consisting of SEQ Identifiers: D1497-D1504, D1564-D1571, and D1584 of Table 3D. In some embodiments, the prime editing composition further comprises a ngRNA that comprises a spacer comprising a sequence selected from the group consisting of SEQ ID NOs. 21130-21137, 21197-21204, and 21217 of Table 3D.

In some embodiments, the PEgRNA comprises a spacer comprising a sequence selected from the group consisting of SEQ Identifier: A721-A722 of Table 3A. In some embodiments, the PEgRNA comprises a spacer comprising a sequence as set forth in SEQ ID NOs. 1069-1070 of Table 3A. In some embodiments, the PEgRNA comprises a PBS comprising a sequence selected from the group consisting of SEQ Identifiers: B2979-B2987 of Table 3B. In some embodiments, the PEgRNA comprises a PBS comprising a sequence selected from the group consisting of SEQ ID NOs. 3530-3536 of Table 3B. In some embodiments, the PEgRNA comprises a PBS comprising a sequence selected from the group consisting of sequences CATACAAG and CATACAAGA as disclosed in Table 3B. In some embodiments, the PEgRNA comprises an editing template comprising a sequence selected from the group consisting of SEQ Identifiers: C13817-C13868 of Table 3C. In some embodiments, the PEgRNA comprises an editing template comprising a sequence selected from the group consisting of SEQ ID NOs: 17843-17894 of Table 3C. In some embodiments, the prime editing composition further comprises a ngRNA that comprises a spacer comprising a sequence selected from the group consisting of SEQ Identifiers: D1450-D1461, D1473-D1478, D1512, D1513, D1516, D1517, D1529, D1536-D1539, D1554, D1556, D1557, D1558, D1559, D1560, D1599, D1607, and D1608 of Table 3D. In some embodiments, the prime editing composition further comprises a ngRNA that comprises a spacer comprising a sequence selected from the group consisting of SEQ ID NOs. 21083-21094, 21106-21111, 21145, 21146, 21149, 21150, 21162, 21169-21172, 21187, 21189-21193, 21232, 21240, and 21241 of Table 3D.

In some embodiments, the PEgRNA comprises a spacer comprising the sequence of SEQ Identifier: A723 of Table 3A. In some embodiments, the PEgRNA comprises a spacer comprising a sequence as set forth in SEQ ID NO. 1071 of Table 3A. In some embodiments, the PEgRNA comprises a PBS comprising a sequence selected from the group consisting of SEQ Identifiers: B2988-B2996 of Table 3B. In some embodiments, the PEgRNA comprises a PBS comprising a sequence selected from the group consisting of SEQ ID NOs. 3537-3543 of Table 3B. In some embodiments, the PEgRNA comprises a PBS comprising a sequence selected from the group consisting of sequences CCATACAA and CCATACAAG as disclosed in Table 3B. In some embodiments, the PEgRNA comprises an editing template comprising a sequence selected from the group consisting of SEQ Identifiers: C13869-C13895 of Table 3C. In some embodiments, the PEgRNA comprises an editing template comprising a sequence selected from the group consisting of SEQ ID NOs: 17895-17921 of Table 3C. In some embodiments, the prime editing composition further comprises a ngRNA that comprises a spacer comprising a sequence selected from the group consisting of SEQ Identifiers: D1450-D1461, D1473-D1478, D1516, D1517, D1529, D1556-D1560, and D1599. In some embodiments, the prime editing composition further comprises a ngRNA that comprises a spacer comprising a sequence selected from the group consisting of SEQ ID NOs. 21083-21094, 21106-21111, 21149, 21150, 21162, 21189-21193, and 21232 of Table 3D.

In some embodiments, the PEgRNA comprises a spacer comprising a sequence selected from the group consisting of SEQ Identifier: A724-A727 of Table 3A. In some embodiments, the PEgRNA comprises a spacer comprising a sequence as set forth in SEQ ID NOs. 1072-1075 of Table 3A. In some embodiments, the PEgRNA comprises a PBS comprising a sequence selected from the group consisting of SEQ Identifiers: B2997-B3005 of Table 3B. In some embodiments, the PEgRNA comprises a PBS comprising a sequence selected from the group consisting of SEQ ID NOs. 3544-3550 of Table 3B. In some embodiments, the PEgRNA comprises a PBS comprising a sequence selected from the group consisting of sequences TTGGCATG and TTGGCATGT as disclosed in Table 3B. In some embodiments, the PEgRNA comprises an editing template comprising a sequence selected from the group consisting of SEQ Identifiers: C13896-C13941 of Table 3C. In some embodiments, the PEgRNA comprises an editing template comprising a sequence selected from the group consisting of SEQ ID NOs: 17922-17967 of Table 3C. In some embodiments, the prime editing composition further comprises a ngRNA that comprises a spacer comprising a sequence selected from the group consisting of SEQ Identifiers: D1435-D1440, D1549-D1553, and D1609-D1614 of Table 3D. In some embodiments, the prime editing composition further comprises a ngRNA that comprises a spacer comprising a sequence selected from the group consisting of SEQ ID NOs. 21068-21073, 21182-21186, and 21242-21247 of Table 3D.

In some embodiments, the PEgRNA comprises a spacer comprising the sequence of SEQ Identifier: A728 of Table 3A. In some embodiments, the PEgRNA comprises a spacer comprising a sequence as set forth in SEQ ID NO. 1076 of Table 3A. In some embodiments, the PEgRNA comprises a PBS comprising a sequence selected from the group consisting of SEQ Identifiers: B3006-B3014 of Table 3B. In some embodiments, the PEgRNA comprises a PBS comprising a sequence selected from the group consisting of SEQ ID NOs. 3551-3557 of Table 3B. In some embodiments, the PEgRNA comprises a PBS comprising a sequence selected from the group consisting of sequences ATGGCCAA and ATGGCCAAC as disclosed in Table 3B. In some embodiments, the PEgRNA comprises an editing template comprising a sequence selected from the group consisting of SEQ Identifiers: C13942-C13958 of Table 3C. In some embodiments, the PEgRNA comprises an editing template comprising a sequence selected from the group consisting of SEQ ID NOs: 17968-17984 of Table 3C. In some embodiments, the prime editing composition further comprises a ngRNA that comprises a spacer comprising a sequence selected from the group consisting of SEQ Identifiers: D1452-D1461, D1473-D1478, D1516, D1517, D1529, and D1556-D1563 of Table 3D. In some embodiments, the prime editing composition further comprises a ngRNA that comprises a spacer comprising a sequence selected from the group consisting of SEQ ID NOs. 21085-21094, 21106-21111, 21149, 21150, 21162, and 21189-21196 of Table 3D.

In some embodiments, the editing target sequence is in exon 23 of the CFTR gene.

In some embodiments, the editing target sequence or the complementary sequence on the CFTR gene thereof comprises a mutation that encodes an amino acid substitution S1255*, F1257L, R1259*, and/or L1260R relative to a wild type CFTR polypeptide set forth in SEQ ID NO: 19, wherein * refers to a non-sense mutation, and wherein the editing template encodes an intended nucleotide edit that corrects the mutation. In some embodiments, the editing target sequence or the complementary sequence on the CFTR gene thereof comprises a T duplication mutation (c.3773dupT) at position 117,642,493, a C→A mutation (c.3764C>A) at position 117,642,484, a duplication of a C (c.3767dupC) at position 117,642,487, a T→G mutation (c.3771T>G) at position 117,642,491, f a G (c.3774dupG) at position 117,642,494, a A→T mutation (c.3775A>T) at position 117,642,495, a T→G mutation (c.3779T>G) at position 117,642,499, and/or a deletion of ACT (c.3780_3782delACT) at position 117,642,500 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide.

In some embodiments, the editing template comprises a wild type CFTR gene sequence. In some embodiments, the editing template comprises one or more intended nucleotide edits compared to the target CFTR gene sequence in exon 23 of the target CFTR gene. Accordingly, in some embodiments, the newly synthesized single stranded DNA comprises one or more intended nucleotide edits compared to the target CFTR gene sequence. In some embodiments, the newly synthesized single stranded DNA encoded by the editing template is capable of correcting the c.3773dupT mutation, the c.3764C>A mutation, the c.3767dupC mutation, the c.3771T>G mutation, the c.3774dupG mutation, the c.3775A>T mutation, the c.3779T>G mutation, and/or the c.3780_3782delACT mutation in the target CFTR gene. In some embodiments, the newly synthesized single stranded DNA encoded by the editing template is capable of correcting all of the c.3773dupT mutation, the c.3764C>A mutation, the c.3767dupC mutation, the c.3771T>G mutation, the c.3774dupG mutation, the c.3775A>T mutation, the c.3779T>G mutation, and the c.3780_3782delACT mutation in the target CFTR gene. In some embodiments, two or more of the c.3773dupT mutation, the c.3764C>A mutation, the c.3767dupC mutation, the c.3771T>G mutation, the c.3774dupG mutation, the c.3775A>T mutation, the c.3779T>G mutation, and the c.3780_3782delACT mutation are located on the same CFTR gene. In some embodiments, the newly synthesized single stranded DNA encoded by the editing template is capable of correcting the c.3773dupT mutation, the c.3764C>A mutation, the c.3767dupC mutation, the c.3771T>G mutation, the c.3774dupG mutation, the c.3775A>T mutation, the c.3779T>G mutation, and the c.3780_3782delACT mutation that are located on different CFTR genes. For example, in some embodiments, a subject may comprise two copies of CFTR genes, each comprising a different mutation selected from the group consisting of the c.3773dupT mutation, the c.3764C>A mutation, the c.3767dupC mutation, the c.3771T>G mutation, the c.3774dupG mutation, the c.3775A>T mutation, the c.3779T>G mutation, and the c.3780_3782delACT mutation, all of which may be corrected by an editing template of a single PEgRNA. In some embodiments, the editing template is capable of correcting one or more, or all of the c.3773dupT mutation, the c.3764C>A mutation, the c.3767dupC mutation, the c.3771T>G mutation, the c.3774dupG mutation, the c.3775A>T mutation, the c.3779T>G mutation, and the c.3780_3782delACT mutation that are in different subjects, wherein each subject may have one or more of the mutations.

In some embodiments, the PEgRNA comprises a spacer comprising a sequence selected from the group consisting of SEQ Identifiers: A729-A730 of Table 3A. In some embodiments, the PEgRNA comprises a spacer comprising a sequence as set forth in SEQ ID NOs. 1077-1078 of Table 3A. In some embodiments, the PEgRNA comprises a PBS comprising a sequence selected from the group consisting of SEQ Identifiers: B3015-B3023 of Table 3B. In some embodiments, the PEgRNA comprises a PBS comprising a sequence selected from the group consisting of SEQ ID NOs. 3558-3564 of Table 3B. In some embodiments, the PEgRNA comprises a PBS comprising a sequence selected from the group consisting of sequences TCGATGGT and TCGATGGTG as disclosed in Table 3B. In some embodiments, the PEgRNA comprises an editing template comprising a sequence selected from the group consisting of SEQ Identifiers: C13959-C13966 of Table 3C. In some embodiments, the PEgRNA comprises an editing template comprising a sequence selected from the group consisting of SEQ ID NOs: 17985-17992 of Table 3C. In some embodiments, the prime editing composition further comprises a ngRNA that comprises a spacer comprising a sequence selected from the group consisting of SEQ Identifiers: D1615-D1650 of Table 3D. In some embodiments, the prime editing composition further comprises a ngRNA that comprises a spacer comprising a sequence selected from the group consisting of SEQ ID NOs. 21248-21283 of Table 3D.

In some embodiments, the PEgRNA comprises a spacer comprising a sequence selected from the group consisting of SEQ Identifiers: A731-A732 of Table 3A. In some embodiments, the PEgRNA comprises a spacer comprising a sequence as set forth in SEQ ID NOs. 1079-1080 of Table 3A. In some embodiments, the PEgRNA comprises a PBS comprising a sequence selected from the group consisting of SEQ Identifiers: B3024-B3032 of Table 3B. In some embodiments, the PEgRNA comprises a PBS comprising a sequence selected from the group consisting of SEQ ID NOs. 3565-3571 of Table 3B. In some embodiments, the PEgRNA comprises a PBS comprising a sequence selected from the group consisting of sequences CGATGGTG and CGATGGTGT as disclosed in Table 3B. In some embodiments, the PEgRNA comprises an editing template comprising a sequence selected from the group consisting of SEQ Identifiers: C13967-C13980 of Table 3C. In some embodiments, the PEgRNA comprises an editing template comprising a sequence selected from the group consisting of SEQ ID NOs: 17993-18006 of Table 3C. In some embodiments, the prime editing composition further comprises a ngRNA that comprises a spacer comprising a sequence selected from the group consisting of SEQ Identifiers: D1624-D1661 of Table 3D. In some embodiments, the prime editing composition further comprises a ngRNA that comprises a spacer comprising a sequence selected from the group consisting of SEQ ID NOs. 21257-21294 of Table 3D.

In some embodiments, the PEgRNA comprises a spacer comprising a sequence selected from the group consisting of SEQ Identifiers: A733-A736 of Table 3A. In some embodiments, the PEgRNA comprises a spacer comprising a sequence as set forth in SEQ ID NOs. 1081-1084 of Table 3A. In some embodiments, the PEgRNA comprises a PBS comprising a sequence selected from the group consisting of SEQ Identifiers: B3033-B3041 of Table 3B. In some embodiments, the PEgRNA comprises a PBS comprising a sequence selected from the group consisting of SEQ ID NOs. 3572-3578 of Table 3B. In some embodiments, the PEgRNA comprises a PBS comprising a sequence selected from the group consisting of sequences CAGTTCTT and CAGTTCTTC as disclosed in Table 3B. In some embodiments, the PEgRNA comprises an editing template comprising a sequence selected from the group consisting of SEQ Identifiers: C13981-C13992 of Table 3C. In some embodiments, the PEgRNA comprises an editing template comprising a sequence selected from the group consisting of SEQ ID NOs: 18007-18018 of Table 3C. In some embodiments, the prime editing composition further comprises a ngRNA that comprises a spacer comprising a sequence selected from the group consisting of SEQ Identifiers: D1662-D1692 of Table 3D. In some embodiments, the prime editing composition further comprises a ngRNA that comprises a spacer comprising a sequence selected from the group consisting of SEQ ID NOs. 21295-21325 of Table 3D.

In some embodiments, the PEgRNA comprises a spacer comprising a sequence selected from the group consisting of SEQ Identifiers: A737-A738 of Table 3A. In some embodiments, the PEgRNA comprises a spacer comprising a sequence as set forth in SEQ ID NOs. 1085-1086 of Table 3A. In some embodiments, the PEgRNA comprises a PBS comprising a sequence selected from the group consisting of SEQ Identifiers: B3042-B3050 of Table 3B. In some embodiments, the PEgRNA comprises a PBS comprising a sequence selected from the group consisting of SEQ ID NOs. 3579-3585 of Table 3B. In some embodiments, the PEgRNA comprises a PBS comprising a sequence selected from the group consisting of sequences TCCAGTTC and TCCAGTTCT as disclosed in Table 3B. In some embodiments, the PEgRNA comprises an editing template comprising a sequence selected from the group consisting of SEQ Identifiers: C13993-C14008 of Table 3C. In some embodiments, the PEgRNA comprises an editing template comprising a sequence selected from the group consisting of SEQ ID NOs: 18019-18034 of Table 3C. In some embodiments, the prime editing composition further comprises a ngRNA that comprises a spacer comprising a sequence selected from the group consisting of SEQ Identifiers: D1671, D1693, D1694, D1695, D1696, D1697, D1698, D1699, D1700, D1701, D1702, D1703, D1704, D1705, D1706, D1707, D1708, D1709, D1710, D1711, D1712, D1713, D1714, D1715, and D1716 of Table 3D. In some embodiments, the prime editing composition further comprises a ngRNA that comprises a spacer comprising a sequence selected from the group consisting of SEQ ID NOs. 21304, and 21326-21349 of Table 3D.

In some embodiments, the PEgRNA comprises a spacer comprising a sequence selected from the group consisting of SEQ Identifiers: A739-A740 of Table 3A. In some embodiments, the PEgRNA comprises a spacer comprising a sequence as set forth in SEQ ID NOs. 1087-1088 of Table 3A. In some embodiments, the PEgRNA comprises a PBS comprising a sequence selected from the group consisting of SEQ Identifiers: B3051-B3059 of Table 3B. In some embodiments, the PEgRNA comprises a PBS comprising a sequence selected from the group consisting of SEQ ID NOs. 3586-3592 of Table 3B. In some embodiments, the PEgRNA comprises a PBS comprising a sequence selected from the group consisting of sequences AAAGCTGA and AAAGCTGAT as disclosed in Table 3B. In some embodiments, the PEgRNA comprises an editing template comprising a sequence selected from the group consisting of SEQ Identifiers: C14009-C14039 of Table 3C. In some embodiments, the PEgRNA comprises an editing template comprising a sequence selected from the group consisting of SEQ ID NOs: 18035-18065 of Table 3C. In some embodiments, the prime editing composition further comprises a ngRNA that comprises a spacer comprising a sequence selected from the group consisting of SEQ Identifiers: D1671, D1701, D1702, D1703, D1704, D1705, D1706, D1707, D1708, D1709, D1710, D1711, D1712, D1713, D1714, D1715, D1716, D1718, D1719, D1720, D1721, D1722, D1723, D1724, D1725, and D1726 of Table 3D. In some embodiments, the prime editing composition further comprises a ngRNA that comprises a spacer comprising a sequence selected from the group consisting of SEQ ID NOs. 21304, 21334-21349, and 21351-21359 of Table 3D.

In some embodiments, the PEgRNA comprises a spacer comprising a sequence selected from the group consisting of SEQ Identifiers: A741-A742 of Table 3A. In some embodiments, the PEgRNA comprises a spacer comprising a sequence as set forth in SEQ ID NOs. 1089-1090 of Table 3A. In some embodiments, the PEgRNA comprises a PBS comprising a sequence selected from the group consisting of SEQ Identifiers: B3060-B3068 of Table 3B. In some embodiments, the PEgRNA comprises a PBS comprising a sequence selected from the group consisting of SEQ ID NOs. 3593-3599 of Table 3B. In some embodiments, the PEgRNA comprises a PBS comprising a sequence selected from the group consisting of sequences CCTGATCC and CCTGATCCA as disclosed in Table 3B. In some embodiments, the PEgRNA comprises an editing template comprising a sequence selected from the group consisting of SEQ Identifiers: C14040-C14065 of Table 3C. In some embodiments, the PEgRNA comprises an editing template comprising a sequence selected from the group consisting of SEQ ID NOs: 18066-18091 of Table 3C. In some embodiments, the prime editing composition further comprises a ngRNA that comprises a spacer comprising a sequence selected from the group consisting of SEQ Identifiers: D1662-D1665, D1668, D1669, D1673-D1680, and D1728-D1731 of Table 3D. In some embodiments, the prime editing composition further comprises a ngRNA that comprises a spacer comprising a sequence selected from the group consisting of SEQ ID NOs. 21295-21298, 21301, 21302, 21306-21313, and 21361-21364 of Table 3D.

In some embodiments, the PEgRNA comprises a spacer comprising the sequence of SEQ Identifier: A743 of Table 3A. In some embodiments, the PEgRNA comprises a spacer comprising a sequence as set forth in SEQ ID NO. 1091 of Table 3A. In some embodiments, the PEgRNA comprises a PBS comprising a sequence selected from the group consisting of SEQ Identifiers: B3069-B3077 of Table 3B. In some embodiments, the PEgRNA comprises a PBS comprising a sequence selected from the group consisting of SEQ ID NOs. 3600-3606 of Table 3B. In some embodiments, the PEgRNA comprises a PBS comprising a sequence selected from the group consisting of sequences CCCTGATC and CCCTGATCC as disclosed in Table 3B. In some embodiments, the PEgRNA comprises an editing template comprising a sequence selected from the group consisting of SEQ Identifiers: C14066-C14079 of Table 3C. In some embodiments, the PEgRNA comprises an editing template comprising a sequence selected from the group consisting of SEQ ID NOs: 18092-18105 of Table 3C. In some embodiments, the prime editing composition further comprises a ngRNA that comprises a spacer comprising a sequence selected from the group consisting of SEQ Identifiers D1671, D1699, D1700, D1701, D1702, D1703, D1704, D1705, D1706, D1707, D1708, D1709, D1710, D1711, D1712, D1713, D1714, D1715, D1716, and D1718 of Table 3D. In some embodiments, the prime editing composition further comprises a ngRNA that comprises a spacer comprising a sequence selected from the group consisting of SEQ ID NOs. 21304, 21332-21349, and 21351 of Table 3D.

In some embodiments, the PEgRNA comprises a spacer comprising a sequence selected from the group consisting of SEQ Identifiers: A744-A745 of Table 3A. In some embodiments, the PEgRNA comprises a spacer comprising a sequence as set forth in SEQ ID NOs. 1092-1093 of Table 3A. In some embodiments, the PEgRNA comprises a PBS comprising a sequence selected from the group consisting of SEQ Identifiers: B3078-B3086 of Table 3B. In some embodiments, the PEgRNA comprises a PBS comprising a sequence selected from the group consisting of SEQ ID NOs. 3607-3613 of Table 3B. In some embodiments, the PEgRNA comprises a PBS comprising a sequence selected from the group consisting of sequences GTGTGTCT and GTGTGTCTT as disclosed in Table 3B. In some embodiments, the PEgRNA comprises an editing template comprising a sequence selected from the group consisting of SEQ Identifiers: C14080-C14081 of Table 3C. In some embodiments, the PEgRNA comprises an editing template comprising a sequence selected from the group consisting of SEQ ID NOs: 18106-18107 of Table 3C. In some embodiments, the prime editing composition further comprises a ngRNA that comprises a spacer comprising a sequence selected from the group consisting of SEQ Identifiers: D1615-D1622, and D1627-D1650 of Table 3D. In some embodiments, the prime editing composition further comprises a ngRNA that comprises a spacer comprising a sequence selected from the group consisting of SEQ ID NOs. 21248-21255, and 21260-21283 of Table 3D.

In some embodiments, the PEgRNA comprises a spacer comprising the sequence of SEQ Identifier: A746 of Table 3A. In some embodiments, the PEgRNA comprises a spacer comprising a sequence as set forth in SEQ ID NO. 1094 of Table 3A. In some embodiments, the PEgRNA comprises a PBS comprising a sequence selected from the group consisting of SEQ Identifiers: B3087-B3095 of Table 3B. In some embodiments, the PEgRNA comprises a PBS comprising a sequence selected from the group consisting of SEQ ID NOs. 3614-3620 of Table 3B. In some embodiments, the PEgRNA comprises a PBS comprising a sequence selected from the group consisting of sequences AAGGAGAA and AAGGAGAAA as disclosed in Table 3B. In some embodiments, the PEgRNA comprises an editing template comprising a sequence selected from the group consisting of SEQ Identifiers: C14082-C14104 of Table 3C. In some embodiments, the PEgRNA comprises an editing template comprising a sequence selected from the group consisting of SEQ ID NOs: 18108-18130 of Table 3C. In some embodiments, the prime editing composition further comprises a ngRNA that comprises a spacer comprising a sequence selected from the group consisting of SEQ Identifiers: D1623-D1650 and D1732-D1734 of Table 3D. In some embodiments, the prime editing composition further comprises a ngRNA that comprises a spacer comprising a sequence selected from the group consisting of SEQ ID NOs. 21256-21283, and 21365-21367 of Table 3D.

In some embodiments, the PEgRNA comprises a spacer comprising a sequence selected from the group consisting of SEQ Identifiers: A747-A748 of Table 3A. In some embodiments, the PEgRNA comprises a spacer comprising a sequence as set forth in SEQ ID NOs. 1095-1096 of Table 3A. In some embodiments, the PEgRNA comprises a PBS comprising a sequence selected from the group consisting of SEQ Identifiers: B3096-B3104 of Table 3B. In some embodiments, the PEgRNA comprises a PBS comprising a sequence selected from the group consisting of SEQ ID NOs. 3621-3627 of Table 3B. In some embodiments, the PEgRNA comprises a PBS comprising a sequence selected from the group consisting of sequences CTGATCCA and CTGATCCAG as disclosed in Table 3B. In some embodiments, the PEgRNA comprises an editing template comprising a sequence selected from the group consisting of SEQ Identifiers: C14105-C14116 of Table 3C. In some embodiments, the PEgRNA comprises an editing template comprising a sequence selected from the group consisting of SEQ ID NOs: 18131-18142 of Table 3C. In some embodiments, the prime editing composition further comprises a ngRNA that comprises a spacer comprising a sequence selected from the group consisting of SEQ Identifiers: D1671, D1699, D1700, D1701, D1702, D1703, D1704, D1705, D1706, D1707, D1708, D1709, D1710, D1711, D1712, D1713, D1714, D1715, D1716, D1718, D1722, D1723, D1724, D1725, and D1733 of Table 3D. In some embodiments, the prime editing composition further comprises a ngRNA that comprises a spacer comprising a sequence selected from the group consisting of SEQ ID NOs. 21304, 21332-21351, 21355-21358, and 21366 of Table 3D.

In some embodiments, the PEgRNA comprises a spacer comprising a sequence selected from the group consisting of SEQ Identifiers: A749-A750 of Table 3A. In some embodiments, the PEgRNA comprises a spacer comprising a sequence as set forth in SEQ ID NOs. 1097-1098 of Table 3A. In some embodiments, the PEgRNA comprises a PBS comprising a sequence selected from the group consisting of SEQ Identifiers: B3105-B3113 of Table 3B. In some embodiments, the PEgRNA comprises a PBS comprising a sequence selected from the group consisting of SEQ ID NOs. 3628-3634 of Table 3B. In some embodiments, the PEgRNA comprises a PBS comprising a sequence selected from the group consisting of sequences ACTGAAGG and ACTGAAGGA as disclosed in Table 3B. In some embodiments, the PEgRNA comprises an editing template comprising a sequence selected from the group consisting of SEQ Identifiers: C14117-C14170 of Table 3C. In some embodiments, the PEgRNA comprises an editing template comprising a sequence selected from the group consisting of SEQ ID NOs: 18143-18196 of Table 3C. In some embodiments, the prime editing composition further comprises a ngRNA that comprises a spacer comprising a sequence selected from the group consisting of SEQ Identifiers: D1736-D1767 of Table 3D. In some embodiments, the prime editing composition further comprises a ngRNA that comprises a spacer comprising a sequence selected from the group consisting of SEQ ID NOs. 21369-21400 of Table 3D.

In some embodiments, the PEgRNA comprises a spacer comprising the sequence of SEQ Identifier: A751 of Table 3A. In some embodiments, the PEgRNA comprises a spacer comprising a sequence as set forth in SEQ ID NO.1099 of Table 3A. In some embodiments, the PEgRNA comprises a PBS comprising a sequence selected from the group consisting of SEQ Identifiers: B3114-B3122 of Table 3B. In some embodiments, the PEgRNA comprises a PBS comprising a sequence selected from the group consisting of SEQ ID NOs. 3635-3641 of Table 3B. In some embodiments, the PEgRNA comprises a PBS comprising a sequence selected from the group consisting of sequences CACTGAAG and CACTGAAGG as disclosed in Table 3B. In some embodiments, the PEgRNA comprises an editing template comprising a sequence selected from the group consisting of SEQ Identifiers: C14171-C14198 of Table 3C. In some embodiments, the PEgRNA comprises an editing template comprising a sequence selected from the group consisting of SEQ ID NOs: 18197-18224 of Table 3C. In some embodiments, the prime editing composition further comprises a ngRNA that comprises a spacer comprising a sequence selected from the group consisting of SEQ Identifiers: D1623-D1650, D1732-D1734, D1768, and D1769 of Table 3D. In some embodiments, the prime editing composition further comprises a ngRNA that comprises a spacer comprising a sequence selected from the group consisting of SEQ ID NOs. 21256-21283, 21365-21367, 21401, and 21402 of Table 3D.

In some embodiments, the PEgRNA comprises a spacer comprising a sequence selected from the group consisting of SEQ Identifiers: A752-A755 of Table 3A. In some embodiments, the PEgRNA comprises a spacer comprising a sequence as set forth in SEQ ID NOs. 1100-1103 of Table 3A. In some embodiments, the PEgRNA comprises a PBS comprising a sequence selected from the group consisting of SEQ Identifiers: B3123-B3131 of Table 3B. In some embodiments, the PEgRNA comprises a PBS comprising a sequence selected from the group consisting of SEQ ID NOs. 3642-3648 of Table 3B. In some embodiments, the PEgRNA comprises a PBS comprising a sequence selected from the group consisting of sequences TGATCCAG and TGATCCAGT as disclosed in Table 3B. In some embodiments, the PEgRNA comprises an editing template comprising a sequence selected from the group consisting of SEQ Identifiers: C14199-C14220 of Table 3C. In some embodiments, the PEgRNA comprises an editing template comprising a sequence selected from the group consisting of SEQ ID NOs: 18225-18246 of Table 3C. In some embodiments, the prime editing composition further comprises a ngRNA that comprises a spacer comprising a sequence selected from the group consisting of SEQ Identifiers: D1662-D1665, D1668, D1669, D1671, D1673-D1676, D1679, D1680, D1683, D1689, D1729, D1731, D1770, D1771, and D1772 of Table 3D. In some embodiments, the prime editing composition further comprises a ngRNA that comprises a spacer comprising a sequence selected from the group consisting of SEQ ID NOs. 21295-21298, 21301, 21302, 21304, 21306-21309, 21312, 21313, 21316, 21322, 21362, 21364, 21403, 21404, and 21405 of Table 3D.

In some embodiments, the PEgRNA comprises a spacer comprising a sequence selected from the group consisting of SEQ Identifier: A756-A757 of Table 3A. In some embodiments, the PEgRNA comprises a spacer comprising a sequence as set forth in SEQ ID NOs. 1104-1105 of Table 3A. In some embodiments, the PEgRNA comprises a PBS comprising a sequence selected from the group consisting of SEQ Identifiers: B3132-B3140 of Table 3B. In some embodiments, the PEgRNA comprises a PBS comprising a sequence selected from the group consisting of SEQ ID NOs. 3649-3655 of Table 3B. In some embodiments, the PEgRNA comprises a PBS comprising a sequence selected from the group consisting of sequences ACAAAGTA and ACAAAGTAC as disclosed in Table 3B. In some embodiments, the PEgRNA comprises an editing template comprising a sequence selected from the group consisting of SEQ Identifiers: C14221-C14274 of Table 3C. In some embodiments, the PEgRNA comprises an editing template comprising a sequence selected from the group consisting of SEQ ID NOs: 18247-18300 of Table 3C. In some embodiments, the prime editing composition further comprises a ngRNA that comprises a spacer comprising a sequence selected from the group consisting of SEQ Identifiers: D1662-D1665, D1668, D1669, D1673-D1676, D1679, D1680, D1729, and D1731 of Table 3D. In some embodiments, the prime editing composition further comprises a ngRNA that comprises a spacer comprising a sequence selected from the group consisting of SEQ ID NOs. 21295-21298, 21301, 21302, 21306-21309, 21312, 21313, 21362, and 21364 of Table 3D.

In some embodiments, the PEgRNA comprises a spacer comprising the sequence of SEQ Identifier: A758 of Table 3A. In some embodiments, the PEgRNA comprises a spacer comprising a sequence as set forth in SEQ ID NO. 1106 of Table 3A. In some embodiments, the PEgRNA comprises a PBS comprising a sequence selected from the group consisting of SEQ Identifiers: B3141-B3149 of Table 3B. In some embodiments, the PEgRNA comprises a PBS comprising a sequence selected from the group consisting of SEQ ID NOs. 3656-3662 of Table 3B. In some embodiments, the PEgRNA comprises a PBS comprising a sequence selected from the group consisting of sequences AACAAAGT and AACAAAGTA as disclosed in Table 3B. In some embodiments, the PEgRNA comprises an editing template comprising a sequence selected from the group consisting of SEQ Identifiers: C14275-C14302 of Table 3C. In some embodiments, the PEgRNA comprises an editing template comprising a sequence selected from the group consisting of SEQ ID NOs: 18301-18328 of Table 3C. In some embodiments, the prime editing composition further comprises a ngRNA that comprises a spacer comprising a sequence selected from the group consisting of SEQ Identifiers: D1671, and D1701-D1720 of Table 3D. In some embodiments, the prime editing composition further comprises a ngRNA that comprises a spacer comprising a sequence selected from the group consisting of SEQ ID NOs. 21304, and 21334-21353 of Table 3D.

In some embodiments, the PEgRNA comprises a spacer comprising a sequence selected from the group consisting of SEQ Identifiers: A759-A760 of Table 3A. In some embodiments, the PEgRNA comprises a spacer comprising a sequence as set forth in SEQ ID NOs. 1107-1108 of Table 3A. In some embodiments, the PEgRNA comprises a PBS comprising a sequence selected from the group consisting of SEQ Identifiers: B3150-B3158 of Table 3B. In some embodiments, the PEgRNA comprises a PBS comprising a sequence selected from the group consisting of SEQ ID NOs. 3663-3669 of Table 3B. In some embodiments, the PEgRNA comprises a PBS comprising a sequence selected from the group consisting of sequences TCTTCCCA and TCTTCCCAA as disclosed in Table 3B. In some embodiments, the PEgRNA comprises an editing template comprising a sequence selected from the group consisting of SEQ Identifiers: C14303-C14304 of Table 3C. In some embodiments, the PEgRNA comprises an editing template comprising a sequence selected from the group consisting of SEQ ID NOs: 18329-18330 of Table 3C. In some embodiments, the prime editing composition further comprises a ngRNA that comprises a spacer comprising a sequence selected from the group consisting of SEQ Identifiers: D1671, D1699-D1716, D1722-D1725, and D1735 of Table 3D. In some embodiments, the prime editing composition further comprises a ngRNA that comprises a spacer comprising a sequence selected from the group consisting of SEQ ID NOs. 21304, 21332-21349, 21355-21358, and 21368 of Table 3D.

In some embodiments, the PEgRNA comprises a spacer comprising a sequence selected from the group consisting of SEQ Identifiers: A761-A763 of Table 3A. In some embodiments, the PEgRNA comprises a spacer comprising a sequence as set forth in SEQ ID NOs. 1109-1113 of Table 3A. In some embodiments, the PEgRNA comprises a PBS comprising a sequence selected from the group consisting of SEQ Identifiers: B3159-B3167 of Table 3B. In some embodiments, the PEgRNA comprises a PBS comprising a sequence selected from the group consisting of SEQ ID NOs. 3670-3676 of Table 3B. In some embodiments, the PEgRNA comprises a PBS comprising a sequence selected from the group consisting of sequences GATGGTGT and GATGGTGTG as disclosed in Table 3B. In some embodiments, the PEgRNA comprises an editing template comprising a sequence selected from the group consisting of SEQ Identifiers: C14305-C14310 of Table 3C. In some embodiments, the PEgRNA comprises an editing template comprising a sequence selected from the group consisting of SEQ ID NOs: 18331-18336 of Table 3C. In some embodiments, the prime editing composition further comprises a ngRNA that comprises a spacer comprising a sequence selected from the group consisting of SEQ Identifiers: D1627, D1637, D1756, D1759, D1761, D1764, and D1773-D1793 of Table 3D. In some embodiments, the prime editing composition further comprises a ngRNA that comprises a spacer comprising a sequence selected from the group consisting of SEQ ID NOs. 21260, 21270, 21389, 21392, 21394, 21397, and 21406-21426 of Table 3D.

In some embodiments, the PEgRNA comprises a spacer comprising a sequence selected from the group consisting of SEQ Identifiers: A764-A765 of Table 3A. In some embodiments, the PEgRNA comprises a spacer comprising a sequence as set forth in SEQ ID NOs. 1112-1113 of Table 3A. In some embodiments, the PEgRNA comprises a PBS comprising a sequence selected from the group consisting of SEQ Identifiers: B3168-B3176 of Table 3B. In some embodiments, the PEgRNA comprises a PBS comprising a sequence selected from the group consisting of SEQ ID NOs. 3677-3683 of Table 3B. In some embodiments, the PEgRNA comprises a PBS comprising a sequence selected from the group consisting of sequences GAGAAATC and GAGAAATCC as disclosed in Table 3B. In some embodiments, the PEgRNA comprises an editing template comprising a sequence selected from the group consisting of SEQ Identifiers: C14311-C14350 of Table 3C. In some embodiments, the PEgRNA comprises an editing template comprising a sequence selected from the group consisting of SEQ ID NOs: 18337-18376 of Table 3C. In some embodiments, the prime editing composition further comprises a ngRNA that comprises a spacer comprising a sequence selected from the group consisting of SEQ Identifiers: D1739-D1748, D1751, D1755-D1764, D1767, D1794, and D1795 of Table 3D. In some embodiments, the prime editing composition further comprises a ngRNA that comprises a spacer comprising a sequence selected from the group consisting of SEQ ID NOs. 21372-21381, 21384, 21388-21397, 21400, 21427, and 21428 of Table 3D.

In some embodiments, the PEgRNA comprises a spacer comprising the sequence of SEQ Identifier: A766 of Table 3A. In some embodiments, the PEgRNA comprises a spacer comprising a sequence as set forth in SEQ ID NO. 1114 of Table 3A. In some embodiments, the PEgRNA comprises a PBS comprising a sequence selected from the group consisting of SEQ Identifiers: B3177-B3185 of Table 3B. In some embodiments, the PEgRNA comprises a PBS comprising a sequence selected from the group consisting of SEQ ID NOs. 3684-3690 of Table 3B. In some embodiments, the PEgRNA comprises a PBS comprising a sequence selected from the group consisting of sequences GGAGAAAT and GGAGAAATC as disclosed in Table 3B. In some embodiments, the PEgRNA comprises an editing template comprising a sequence selected from the group consisting of SEQ Identifiers: C14351-C14371 of Table 3C. In some embodiments, the PEgRNA comprises an editing template comprising a sequence selected from the group consisting of SEQ ID NOs: 18377-18397 of Table 3C. In some embodiments, the prime editing composition further comprises a ngRNA that comprises a spacer comprising a sequence selected from the group consisting of SEQ Identifiers: D1623-D1650, D1733, and D1734 of Table 3D. In some embodiments, the prime editing composition further comprises a ngRNA that comprises a spacer comprising a sequence selected from the group consisting of SEQ ID NOs. 21256-21283, 21366, and 21367 of Table 3D.

In some embodiments, the PEgRNA comprises a spacer comprising a sequence selected from the group consisting of SEQ Identifiers: A767-A770 of Table 3A. In some embodiments, the PEgRNA comprises a spacer comprising a sequence as set forth in SEQ ID NOs. 1115-1118 of Table 3A. In some embodiments, the PEgRNA comprises a PBS comprising a sequence selected from the group consisting of SEQ Identifiers: B3186-B3194 of Table 3B. In some embodiments, the PEgRNA comprises a PBS comprising a sequence selected from the group consisting of SEQ ID NOs. 3691-3697 of Table 3B. In some embodiments, the PEgRNA comprises a PBS comprising a sequence selected from the group consisting of sequences CCAGTTCT and CCAGTTCTT as disclosed in Table 3B. In some embodiments, the PEgRNA comprises an editing template comprising a sequence selected from the group consisting of SEQ Identifiers: C14372-C14378 of Table 3C. In some embodiments, the PEgRNA comprises an editing template comprising a sequence selected from the group consisting of SEQ ID NOs: 18398-18404 of Table 3C. In some embodiments, the prime editing composition further comprises a ngRNA that comprises a spacer comprising a sequence selected from the group consisting of SEQ Identifiers: D1663, D1665, D1668, D1670, D1671, D1672, and D1681-D1716 of Table 3D. In some embodiments, the prime editing composition further comprises a ngRNA that comprises a spacer comprising a sequence selected from the group consisting of SEQ ID NOs. 21296, 21298, 21301, 21303-21305, and 21314-21349 of Table 3D.

In some embodiments, the PEgRNA comprises a spacer comprising the sequence of SEQ Identifier: A771 of Table 3A. In some embodiments, the PEgRNA comprises a spacer comprising a sequence as set forth in SEQ ID NO. 1119 of Table 3A. In some embodiments, the PEgRNA comprises a PBS comprising a sequence selected from the group consisting of SEQ Identifiers: B3195-B3203 of Table 3B. In some embodiments, the PEgRNA comprises a PBS comprising a sequence selected from the group consisting of SEQ ID NOs. 3698-3704 of Table 3B. In some embodiments, the PEgRNA comprises a PBS comprising a sequence selected from the group consisting of sequences TACTCTTC and TACTCTTCC as disclosed in Table 3B. In some embodiments, the PEgRNA comprises an editing template comprising a sequence selected from the group consisting of SEQ Identifiers: C14379-C14399 of Table 3C. In some embodiments, the PEgRNA comprises an editing template comprising a sequence selected from the group consisting of SEQ ID NOs: 18405-18425 of Table 3C. In some embodiments, the prime editing composition further comprises a ngRNA that comprises a spacer comprising a sequence selected from the group consisting of SEQ Identifiers: D1671, and D1701-D1719 of Table 3D. In some embodiments, the prime editing composition further comprises a ngRNA that comprises a spacer comprising a sequence selected from the group consisting of SEQ ID NOs. 21304, and 21334-21352 of Table 3D.

In some embodiments, the PEgRNA comprises a spacer comprising a sequence selected from the group consisting of SEQ Identifiers: A772-A773 of Table 3A. In some embodiments, the PEgRNA comprises a spacer comprising a sequence as set forth in SEQ ID NOs. 1120-1121 of Table 3A. In some embodiments, the PEgRNA comprises a PBS comprising a sequence selected from the group consisting of SEQ Identifiers: B3204-B3212 of Table 3B. In some embodiments, the PEgRNA comprises a PBS comprising a sequence selected from the group consisting of SEQ ID NOs. 3705-3711 of Table 3B. In some embodiments, the PEgRNA comprises a PBS comprising a sequence selected from the group consisting of sequences CTTCCCAA and CTTCCCAAG as disclosed in Table 3B. In some embodiments, the PEgRNA comprises an editing template comprising the sequence of SEQ Identifier: C14400 of Table 3C. In some embodiments, the PEgRNA comprises an editing template comprising a sequence selected from the group consisting of SEQ ID NOs: 18426 of Table 3C. In some embodiments, the prime editing composition further comprises a ngRNA that comprises a spacer comprising a sequence selected from the group consisting of SEQ Identifiers: D1671, and D1693-D1716 of Table 3D. In some embodiments, the prime editing composition further comprises a ngRNA that comprises a spacer comprising a sequence selected from the group consisting of SEQ ID NOs. 21304, and 21326-21349 of Table 3D.

In some embodiments, the PEgRNA comprises a spacer comprising a sequence selected from the group consisting of SEQ Identifiers: A774-A775 of Table 3A. In some embodiments, the PEgRNA comprises a spacer comprising a sequence as set forth in SEQ ID NOs. 1122-1123 of Table 3A. In some embodiments, the PEgRNA comprises a PBS comprising a sequence selected from the group consisting of SEQ Identifiers: B3213-B3221 of Table 3B. In some embodiments, the PEgRNA comprises a PBS comprising a sequence selected from the group consisting of SEQ ID NOs. 3712-3718 of Table 3B. In some embodiments, the PEgRNA comprises a PBS comprising a sequence selected from the group consisting of sequences ATCCAGTT and ATCCAGTTC as disclosed in Table 3B. In some embodiments, the PEgRNA comprises an editing template comprising a sequence selected from the group consisting of SEQ Identifiers: C14401-C14409 of Table 3C. In some embodiments, the PEgRNA comprises an editing template comprising a sequence selected from the group consisting of SEQ ID NOs: 18427-18435 of Table 3C. In some embodiments, the prime editing composition further comprises a ngRNA that comprises a spacer comprising a sequence selected from the group consisting of SEQ Identifiers: D1671, D1699-D1718, D1722-D1725, and D1735 of Table 3D. In some embodiments, the prime editing composition further comprises a ngRNA that comprises a spacer comprising a sequence selected from the group consisting of SEQ ID NOs. 21304, 21332-21351, 21355-21358, and 21368 of Table 3D.

In some embodiments, the PEgRNA comprises a spacer comprising a sequence selected from the group consisting of SEQ Identifiers: A776-A777 of Table 3A. In some embodiments, the PEgRNA comprises a spacer comprising a sequence as set forth in SEQ ID NOs. 1124-1125 of Table 3A. In some embodiments, the PEgRNA comprises a PBS comprising a sequence selected from the group consisting of SEQ Identifiers: B3222-B3230 of Table 3B. In some embodiments, the PEgRNA comprises a PBS comprising a sequence selected from the group consisting of SEQ ID NOs. 3719-3725 of Table 3B. In some embodiments, the PEgRNA comprises a PBS comprising a sequence selected from the group consisting of sequences AAAAAGCT and AAAAAGCTG as disclosed in Table 3B. In some embodiments, the PEgRNA comprises an editing template comprising a sequence selected from the group consisting of SEQ Identifiers: C14410-C14440 of Table 3C. In some embodiments, the PEgRNA comprises an editing template comprising a sequence selected from the group consisting of SEQ ID NOs: 18436-18466 of Table 3C. In some embodiments, the prime editing composition further comprises a ngRNA that comprises a spacer comprising a sequence selected from the group consisting of SEQ Identifiers: D1671, D1702-D1727, and D1796 of Table 3D. In some embodiments, the prime editing composition further comprises a ngRNA that comprises a spacer comprising a sequence selected from the group consisting of SEQ ID NOs. 21304, 21335-21360, 21429 of Table 3D.

In some embodiments, the PEgRNA comprises a spacer comprising a sequence selected from the group consisting of SEQ Identifiers: A778-A779 of Table 3A. In some embodiments, the PEgRNA comprises a spacer comprising a sequence as set forth in SEQ ID NO. 1126-1127 of Table 3A. In some embodiments, the PEgRNA comprises a PBS comprising a sequence selected from the group consisting of SEQ Identifiers: B3231-B3239 of Table 3B. In some embodiments, the PEgRNA comprises a PBS comprising a sequence selected from the group consisting of SEQ ID NOs. 3726-3732 of Table 3B. In some embodiments, the PEgRNA comprises a PBS comprising a sequence selected from the group consisting of sequences AACACTGA and AACACTGAA as disclosed in Table 3B. In some embodiments, the PEgRNA comprises an editing template comprising a sequence selected from the group consisting of SEQ Identifiers: C14441-C14500 of Table 3C. In some embodiments, the PEgRNA comprises an editing template comprising a sequence selected from the group consisting of SEQ ID NOs: 18467-18526 of Table 3C. In some embodiments, the prime editing composition further comprises a ngRNA that comprises a spacer comprising a sequence selected from the group consisting of SEQ Identifiers: D1739-D1751, D1755-D1767, and D1797-D1800 of Table 3D. In some embodiments, the prime editing composition further comprises a ngRNA that comprises a spacer comprising a sequence selected from the group consisting of SEQ ID NOs. 21372-21384, 21388-21400, and 21430-21433 of Table 3D.

In some embodiments, the PEgRNA comprises a spacer comprising the sequence of SEQ ID No: A780 of Table 3A. In some embodiments, the PEgRNA comprises a spacer comprising a sequence as set forth in SEQ ID NO. 1128 of Table 3A. In some embodiments, the PEgRNA comprises a PBS comprising a sequence selected from the group consisting of SEQ Identifiers: B3240-B3248 of Table 3B. In some embodiments, the PEgRNA comprises a PBS comprising a sequence selected from the group consisting of SEQ ID NOs. 3733-3739 of Table 3B. In some embodiments, the PEgRNA comprises a PBS comprising a sequence selected from the group consisting of sequences GAACACTG and GAACACTGA as disclosed in Table 3B. In some embodiments, the PEgRNA comprises an editing template comprising a sequence selected from the group consisting of SEQ Identifiers: C14501-C14531 of Table 3C. In some embodiments, the PEgRNA comprises an editing template comprising a sequence selected from the group consisting of SEQ ID NOs: 18527-18557 of Table 3C. In some embodiments, the prime editing composition further comprises a ngRNA that comprises a spacer comprising a sequence selected from the group consisting of SEQ Identifiers: D1623-D1650, D1732-D1734, D1768, D1769, and D1801 of Table 3D. In some embodiments, the prime editing composition further comprises a ngRNA that comprises a spacer comprising a sequence selected from the group consisting of SEQ ID NOs. 21256-21283, 21365-21368, 21401, 21402, and 21434 of Table 3D.

In some embodiments, the PEgRNA comprises a spacer comprising a sequence selected from the group consisting of SEQ Identifiers: A781-A782 of Table 3A. In some embodiments, the PEgRNA comprises a spacer comprising a sequence as set forth in SEQ ID NOs. 1129-1130 of Table 3A. In some embodiments, the PEgRNA comprises a PBS comprising a sequence selected from the group consisting of SEQ Identifiers: B3249-B3257 of Table 3B. In some embodiments, the PEgRNA comprises a PBS comprising a sequence selected from the group consisting of SEQ ID NOs. 3740-3746 of Table 3B. In some embodiments, the PEgRNA comprises a PBS comprising a sequence selected from the group consisting of sequences AAAAGCTG and AAAAGCTGA as disclosed in Table 3B. In some embodiments, the PEgRNA comprises an editing template comprising a sequence selected from the group consisting of SEQ Identifiers: C14532-C14562 of Table 3C. In some embodiments, the PEgRNA comprises an editing template comprising a sequence selected from the group consisting of SEQ ID NOs: 18558-18588 of Table 3C. In some embodiments, the prime editing composition further comprises a ngRNA that comprises a spacer comprising a sequence selected from the group consisting of SEQ Identifiers: D1662-D1665, D1668, D1669, D1673-D1676, D1679, D1680, D1729, D1731, D1802, and D1803 of Table 3D. In some embodiments, the prime editing composition further comprises a ngRNA that comprises a spacer comprising a sequence selected from the group consisting of SEQ ID NOs. 21295-21298, 21301, 21302, 21306-21309, 21312, 21313, 21362, 21364, 21435, and 21436 of Table 3D.

In some embodiments, the editing target sequence or the complementary sequence on the CFTR gene thereof comprises a mutation that encodes an amino acid substitution W1282*, W1282G, W1282C, and/or R1283M relative to a wild type CFTR polypeptide set forth in SEQ ID NO: 19, and wherein the editing template encodes an intended nucleotide edit that corrects the mutation. In some embodiments, the editing target sequence or the complementary sequence on the CFTR gene thereof comprises a G→A mutation (c.3846G>A) at position 117,642,566, a T→G mutation (c.3844T>G) at position 117,642,564, a G→T mutation (c.3846G>T) at position 117,642,566, a G→T mutation (c.3848G>T) at position 117,642,568, and/or a deletion of C (c.3855delC) at position 117,642,575 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide. In some embodiments, the editing template comprises a wild type CFTR gene sequence. Accordingly, in some embodiments, the newly synthesized single stranded DNA encoded by the editing template is capable of correcting the c.3846G>A mutation, the c.3844T>G mutation, the c.3846G>T mutation, the c.3848G>T mutation, and/or the c.3855delC mutation in the target CFTR gene. In some embodiments, the newly synthesized single stranded DNA encoded by the editing template is capable of correcting all of the c.3846G>A mutation, the c.3844T>G mutation, the c.3846G>T mutation, the c.3848G>T mutation, and the c.3855delC mutation in the target CFTR gene. In some embodiments, two or more of the c.3846G>A mutation, the c.3844T>G mutation, the c.3846G>T mutation, the c.3848G>T mutation, and the c.3855delC mutation are located on the same CFTR gene. In some embodiments, the newly synthesized single stranded DNA encoded by the editing template is capable of correcting the c.3846G>A mutation, the c.3844T>G mutation, the c.3846G>T mutation, the c.3848G>T mutation, and the c.3855delC mutation that are located on different CFTR genes. For example, in some embodiments, a subject may comprise two copies of CFTR genes, each comprising a different mutation selected from the group consisting of the c.3846G>A mutation, the c.3844T>G mutation, the c.3846G>T mutation, the c.3848G>T mutation, and the c.3855delC mutation, all of which may be corrected by an editing template of a single PEgRNA. In some embodiments, the editing template is capable of correcting one or more, or all of the c.3846G>A mutation, the c.3844T>G mutation, the c.3846G>T mutation, the c.3848G>T mutation, and the c.3855delC mutation that are in different subjects, wherein each subject may have one or more of the mutations.

In some embodiments, the PEgRNA comprises a spacer comprising the sequence of SEQ Identifier: A783 of Table 3A. In some embodiments, the PEgRNA comprises a spacer comprising a sequence as set forth in SEQ ID NO. 1131 of Table 3A. In some embodiments, the PEgRNA comprises a PBS comprising a sequence selected from the group consisting of SEQ Identifiers: B3258-B3266 of Table 3B. In some embodiments, the PEgRNA comprises a PBS comprising a sequence selected from the group consisting of SEQ ID NOs. 3747-3753 of Table 3B. In some embodiments, the PEgRNA comprises a PBS comprising a sequence selected from the group consisting of sequences GATCTGGA and GATCTGGAT as disclosed in Table 3B. In some embodiments, the PEgRNA comprises an editing template comprising the sequence of SEQ Identifiers: C14563 of Table 3C. In some embodiments, the PEgRNA comprises an editing template comprising a sequence selected from the group consisting of SEQ ID NOs: 18589 of Table 3C. In some embodiments, the prime editing composition further comprises a ngRNA that comprises a spacer comprising a sequence selected from the group consisting of SEQ Identifiers: D1671, D1708-D1721, D1796, D1804-D1809 of Table 3D. In some embodiments, the prime editing composition further comprises a ngRNA that comprises a spacer comprising a sequence selected from the group consisting of SEQ ID NOs. 21304, 21341-21354, 21429, 21437-21442 of Table 3D.

In some embodiments, the PEgRNA comprises a spacer comprising a sequence selected from the group consisting of SEQ Identifiers: A784-A785 of Table 3A. In some embodiments, the PEgRNA comprises a spacer comprising a sequence as set forth in SEQ ID NOs. 1132-1133 of Table 3A. In some embodiments, the PEgRNA comprises a PBS comprising a sequence selected from the group consisting of SEQ Identifiers: B3267-B3275 of Table 3B. In some embodiments, the PEgRNA comprises a PBS comprising a sequence selected from the group consisting of SEQ ID NOs. 3754-3760 of Table 3B. In some embodiments, the PEgRNA comprises a PBS comprising a sequence selected from the group consisting of sequences GTGAGCAA and GTGAGCAAA as disclosed in Table 3B. In some embodiments, the PEgRNA comprises an editing template comprising a sequence selected from the group consisting of SEQ Identifiers: C14564-C14575 of Table 3C. In some embodiments, the PEgRNA comprises an editing template comprising a sequence selected from the group consisting of SEQ ID NOs: 18590-18601 of Table 3C. In some embodiments, the prime editing composition further comprises a ngRNA that comprises a spacer comprising a sequence selected from the group consisting of SEQ Identifiers: D1645-D1650, D1657, D1658, and D1810-D1818 of Table 3D. In some embodiments, the prime editing composition further comprises a ngRNA that comprises a spacer comprising a sequence selected from the group consisting of SEQ ID NOs. 21278-21283, 21290, 21291, and 21443-21451 of Table 3D.

In some embodiments, the PEgRNA comprises a spacer comprising the sequence of SEQ Identifier: A786 of Table 3A. In some embodiments, the PEgRNA comprises a spacer comprising a sequence as set forth in SEQ ID NO. 1134 of Table 3A. In some embodiments, the PEgRNA comprises a PBS comprising a sequence selected from the group consisting of SEQ Identifiers: B3276-B3284 of Table 3B. In some embodiments, the PEgRNA comprises a PBS comprising a sequence selected from the group consisting of SEQ ID NOs. 3761-3767 of Table 3B. In some embodiments, the PEgRNA comprises a PBS comprising a sequence selected from the group consisting of sequences GGTGAGCA and GGTGAGCAA as disclosed in Table 3B. In some embodiments, the PEgRNA comprises an editing template comprising a sequence selected from the group consisting of SEQ Identifiers: C14576-C14588 of Table 3C. In some embodiments, the PEgRNA comprises an editing template comprising a sequence selected from the group consisting of SEQ ID NOs: 18602-18614 of Table 3C. In some embodiments, the prime editing composition further comprises a ngRNA that comprises a spacer comprising a sequence selected from the group consisting of SEQ Identifiers: D1644-D1650, and D1810-D1818 of Table 3D. In some embodiments, the prime editing composition further comprises a ngRNA that comprises a spacer comprising a sequence selected from the group consisting of SEQ ID NOs. 21277-21283, and 21443-21451 of Table 3D.

In some embodiments, the PEgRNA comprises a spacer comprising the sequence of SEQ Identifiers: A787-A788 of Table 3A. In some embodiments, the PEgRNA comprises a spacer comprising a sequence as set forth in SEQ ID NOs. 1135-1136 of Table 3A. In some embodiments, the PEgRNA comprises a PBS comprising a sequence selected from the group consisting of SEQ Identifiers: B3285-B3293 of Table 3B. In some embodiments, the PEgRNA comprises a PBS comprising a sequence selected from the group consisting of SEQ ID NOs. 3768-3774 of Table 3B. In some embodiments, the PEgRNA comprises a PBS comprising a sequence selected from the group consisting of sequences CTGTTGCA and CTGTTGCAA as disclosed in Table 3B. In some embodiments, the PEgRNA comprises an editing template comprising a sequence selected from the group consisting of SEQ Identifiers: C14589-C14650 of Table 3C. In some embodiments, the PEgRNA comprises an editing template comprising a sequence selected from the group consisting of SEQ ID NOs: 18615-18676 of Table 3C. In some embodiments, the prime editing composition further comprises a ngRNA that comprises a spacer comprising a sequence selected from the group consisting of SEQ Identifiers: D1698, D1714-D1716, D1718-D1721, D1796, D1804-D1809, and D1819-D1824 of Table 3D. In some embodiments, the prime editing composition further comprises a ngRNA that comprises a spacer comprising a sequence selected from the group consisting of SEQ ID NOs. 21331, 21347-21349, 21351-21354, 21429, 21437-21442, and 21452-21457 of Table 3D.

In some embodiments, the PEgRNA comprises a spacer comprising a sequence selected from the group consisting of SEQ Identifiers: A789-A790 of Table 3A. In some embodiments, the PEgRNA comprises a spacer comprising a sequence as set forth in SEQ ID NOs. 1137-1138 of Table 3A. In some embodiments, the PEgRNA comprises a PBS comprising a sequence selected from the group consisting of SEQ Identifiers: B3294-B3302 of Table 3B. In some embodiments, the PEgRNA comprises a PBS comprising a sequence selected from the group consisting of SEQ ID NOs. 3775-3781 of Table 3B. In some embodiments, the PEgRNA comprises a PBS comprising a sequence selected from the group consisting of sequences GAGTGATA and GAGTGATAC as disclosed in Table 3B. In some embodiments, the PEgRNA comprises an editing template comprising a sequence selected from the group consisting of SEQ Identifiers: C14651-C14702 of Table 3C. In some embodiments, the PEgRNA comprises an editing template comprising a sequence selected from the group consisting of SEQ ID NOs: 18677-18728 of Table 3C. In some embodiments, the prime editing composition further comprises a ngRNA that comprises a spacer comprising a sequence selected from the group consisting of SEQ Identifiers: D1641-D1650, D1656-D1658, D1810-D1816, D1825, and D1826 of Table 3D. In some embodiments, the prime editing composition further comprises a ngRNA that comprises a spacer comprising a sequence selected from the group consisting of SEQ ID NOs. 21274-21283, 21289-21291, 21443-21449, 21458, and 21459 of Table 3D.

In some embodiments, the PEgRNA comprises a spacer comprising the sequence of SEQ Identifier: A791 of Table 3A. In some embodiments, the PEgRNA comprises a spacer comprising a sequence as set forth in SEQ ID NO. 1139 of Table 3A. In some embodiments, the PEgRNA comprises a PBS comprising a sequence selected from the group consisting of SEQ Identifiers: B3303-B3311 of Table 3B. In some embodiments, the PEgRNA comprises a PBS comprising a sequence selected from the group consisting of SEQ ID NOs. 3782-3788 of Table 3B. In some embodiments, the PEgRNA comprises a PBS comprising a sequence selected from the group consisting of sequences GGAGTGAT and GGAGTGATA as disclosed in Table 3B. In some embodiments, the PEgRNA comprises an editing template comprising a sequence selected from the group consisting of SEQ Identifiers: C14703-C14729 of Table 3C. In some embodiments, the PEgRNA comprises an editing template comprising a sequence selected from the group consisting of SEQ ID NOs: 18729-18755 of Table 3C. In some embodiments, the prime editing composition further comprises a ngRNA that comprises a spacer comprising a sequence selected from the group consisting of SEQ Identifiers: D1641-D1650, D1810-1816, and D1827 of Table 3D. In some embodiments, the prime editing composition further comprises a ngRNA that comprises a spacer comprising a sequence selected from the group consisting of SEQ ID NOs. of Table 3D.

In some embodiments, the PEgRNA comprises a spacer comprising the sequence of SEQ Identifier: A792 of Table 3A. In some embodiments, the PEgRNA comprises a spacer comprising a sequence as set forth in SEQ ID NO. 1140 of Table 3A. In some embodiments, the PEgRNA comprises a PBS comprising a sequence selected from the group consisting of SEQ Identifiers: B3312-B3320 of Table 3B. In some embodiments, the PEgRNA comprises a PBS comprising a sequence selected from the group consisting of SEQ ID NOs. 3789-3795 of Table 3B. In some embodiments, the PEgRNA comprises a PBS comprising a sequence selected from the group consisting of sequences CATCGATC and CATCGATCT as disclosed in Table 3B. In some embodiments, the PEgRNA comprises an editing template comprising a sequence selected from the group consisting of SEQ Identifiers: C14730-C14734 of Table 3C. In some embodiments, the PEgRNA comprises an editing template comprising a sequence selected from the group consisting of SEQ ID NOs: 18756-18760 of Table 3C. In some embodiments, the prime editing composition further comprises a ngRNA that comprises a spacer comprising a sequence selected from the group consisting of SEQ Identifiers: D1671, D1708-D1721, D1796, and D1804-D1809 of Table 3D. In some embodiments, the prime editing composition further comprises a ngRNA that comprises a spacer comprising a sequence selected from the group consisting of SEQ ID NOs. 21304, 21341-21354, 21429, and 21437-21442 of Table 3D.

In some embodiments, the PEgRNA comprises a spacer comprising a sequence selected from the group consisting of SEQ Identifiers: A793-A794 of Table 3A. In some embodiments, the PEgRNA comprises a spacer comprising a sequence as set forth in SEQ ID NOs. 1141-1142 of Table 3A. In some embodiments, the PEgRNA comprises a PBS comprising a sequence selected from the group consisting of SEQ Identifiers: B3321-B3329 of Table 3B. In some embodiments, the PEgRNA comprises a PBS comprising a sequence selected from the group consisting of SEQ ID NOs. 3796-3802 of Table 3B. In some embodiments, the PEgRNA comprises a PBS comprising a sequence selected from the group consisting of sequences GACACACC and GACACACCA as disclosed in Table 3B. In some embodiments, the PEgRNA comprises an editing template comprising a sequence selected from the group consisting of SEQ Identifiers: C14735-C14746 of Table 3C. In some embodiments, the PEgRNA comprises an editing template comprising a sequence selected from the group consisting of SEQ ID NOs: 18761-18772 of Table 3C. In some embodiments, the prime editing composition further comprises a ngRNA that comprises a spacer comprising a sequence selected from the group consisting of SEQ Identifiers: D1671, D1708-D1721, D1724-D1727, D1796, D1804-D1809, D1820, D1828, and D1829 of Table 3D. In some embodiments, the prime editing composition further comprises a ngRNA that comprises a spacer comprising a sequence selected from the group consisting of SEQ ID NOs. 21304, 21341-21354, 21357-21360, 21429, 21437-21442, 21453, 21461, and 21462 of Table 3D.

In some embodiments, the PEgRNA comprises a spacer comprising a sequence selected from the group consisting of SEQ Identifiers: A795-A796 of Table 3A. In some embodiments, the PEgRNA comprises a spacer comprising a sequence as set forth in SEQ ID NOs. 1143-1144 of Table 3A. In some embodiments, the PEgRNA comprises a PBS comprising a sequence selected from the group consisting of SEQ Identifiers: B3330-B3338 of Table 3B. In some embodiments, the PEgRNA comprises a PBS comprising a sequence selected from the group consisting of SEQ ID NOs. 3803-3809 of Table 3B. In some embodiments, the PEgRNA comprises a PBS comprising a sequence selected from the group consisting of sequences ACTGTTGC and ACTGTTGCA as disclosed in Table 3B. In some embodiments, the PEgRNA comprises an editing template comprising a sequence selected from the group consisting of SEQ Identifiers: C14747-C14777 of Table 3C. In some embodiments, the PEgRNA comprises an editing template comprising a sequence selected from the group consisting of SEQ ID NOs: 18773-18803 of Table 3C. In some embodiments, the prime editing composition further comprises a ngRNA that comprises a spacer comprising a sequence selected from the group consisting of SEQ Identifiers: D1714-D1716, D1718-D1721, D1725, D1726, D1727, D1796, D1804-D1809, 1820, D1821-D1824, and D1828-D1831 of Table 3D. In some embodiments, the prime editing composition further comprises a ngRNA that comprises a spacer comprising a sequence selected from the group consisting of SEQ ID NOs. 21347-21349, 21351-21354, 21358, 21359, 21360, 21429, 21437-21442, 21453, 21454-21457, and 21461-21464 of Table 3D.

In some embodiments, the PEgRNA comprises a spacer comprising a sequence selected from the group consisting of SEQ Identifiers: A797-A799 of Table 3A. In some embodiments, the PEgRNA comprises a spacer comprising a sequence as set forth in SEQ ID NOs. 1145-1147 of Table 3A. In some embodiments, the PEgRNA comprises a PBS comprising a sequence selected from the group consisting of SEQ Identifiers: B3339-B3347 of Table 3B. In some embodiments, the PEgRNA comprises a PBS comprising a sequence selected from the group consisting of SEQ ID NOs. 3810-3816 of Table 3B. In some embodiments, the PEgRNA comprises a PBS comprising a sequence selected from the group consisting of sequences ACACCATC and ACACCATCG as disclosed in Table 3B. In some embodiments, the PEgRNA comprises an editing template comprising a sequence selected from the group consisting of SEQ Identifiers: C14778-C14786 of Table 3C. In some embodiments, the PEgRNA comprises an editing template comprising a sequence selected from the group consisting of SEQ ID NOs: 18804-18812 of Table 3C. In some embodiments, the prime editing composition further comprises a ngRNA that comprises a spacer comprising a sequence selected from the group consisting of SEQ Identifiers: D1665, D1668, D1671, D1672, D1683-D1686, D1689-D1692, D1832, D1833, and D1834 of Table 3D. In some embodiments, the prime editing composition further comprises a ngRNA that comprises a spacer comprising a sequence selected from the group consisting of SEQ ID NOs. 21298, 21301, 21304, 21305, 21316-21319, 21322-21325, and 21465-21467 of Table 3D.

In some embodiments, the PEgRNA comprises a spacer comprising a sequence selected from the group consisting of SEQ Identifiers: A800-A803 of Table 3A. In some embodiments, the PEgRNA comprises a spacer comprising a sequence as set forth in SEQ ID NOs. 1148-1151 of Table 3A. In some embodiments, the PEgRNA comprises a PBS comprising a sequence selected from the group consisting of SEQ Identifiers: B3348-B3356 of Table 3B. In some embodiments, the PEgRNA comprises a PBS comprising a sequence selected from the group consisting of SEQ ID NOs. 3817-3823 of Table 3B. In some embodiments, the PEgRNA comprises a PBS comprising a sequence selected from the group consisting of sequences TGTTGCAA and TGTTGCAAA as disclosed in Table 3B. In some embodiments, the PEgRNA comprises an editing template comprising a sequence selected from the group consisting of SEQ Identifiers: C14787-C14848 of Table 3C. In some embodiments, the PEgRNA comprises an editing template comprising a sequence selected from the group consisting of SEQ ID NOs: 18813-18874 of Table 3C. In some embodiments, the prime editing composition further comprises a ngRNA that comprises a spacer comprising a sequence selected from the group consisting of SEQ Identifiers: D1668, D1669, D1679, D1680, D1686, D1692, D1729, D1731, D1802, D1803, and D1832-D1848 of Table 3D. In some embodiments, the prime editing composition further comprises a ngRNA that comprises a spacer comprising a sequence selected from the group consisting of SEQ ID NOs. 21301, 21302, 21312, 21313, 21319, 21325, 21362, 21364, 21435, 21436, and 21465-21481 of Table 3D

In some embodiments, the PEgRNA comprises a spacer comprising a sequence selected from the group consisting of SEQ Identifiers: A804-A807 of Table 3A. In some embodiments, the PEgRNA comprises a spacer comprising a sequence as set forth in SEQ ID NOs. 1152-1155 of Table 3A. In some embodiments, the PEgRNA comprises a PBS comprising a sequence selected from the group consisting of SEQ Identifiers: B3357-B3365 of Table 3B. In some embodiments, the PEgRNA comprises a PBS comprising a sequence selected from the group consisting of SEQ ID NOs. 3824-3830 of Table 3B. In some embodiments, the PEgRNA comprises a PBS comprising a sequence selected from the group consisting of sequences AGTGATAC and AGTGATACC as disclosed in Table 3B. In some embodiments, the PEgRNA comprises an editing template comprising a sequence selected from the group consisting of SEQ Identifiers: C14849-C14898 of Table 3C. In some embodiments, the PEgRNA comprises an editing template comprising a sequence selected from the group consisting of SEQ ID NOs: 18875-18924 of Table 3C. In some embodiments, the prime editing composition further comprises a ngRNA that comprises a spacer comprising a sequence selected from the group consisting of SEQ Identifiers: D1743-D1748, D1759-D1764, D1778-D1780, D1787-D1789, and D1849-D1852 of Table 3D. In some embodiments, the prime editing composition further comprises a ngRNA that comprises a spacer comprising a sequence selected from the group consisting of SEQ ID NOs. 21376-21381, 21392-21397, 21411, 21413, 21420-21422, and 21482-21485 of Table 3D.

In some embodiments, the PEgRNA comprises a spacer comprising a sequence selected from the group consisting of SEQ Identifiers: A808-A809 of Table 3A. In some embodiments, the PEgRNA comprises a spacer comprising a sequence as set forth in SEQ ID NOs. 1156-1157 of Table 3A. In some embodiments, the PEgRNA comprises a PBS comprising a sequence selected from the group consisting of SEQ Identifiers: B3366-B3374 of Table 3B. In some embodiments, the PEgRNA comprises a PBS comprising a sequence selected from the group consisting of SEQ ID NOs. 3831-3837 of Table 3B. In some embodiments, the PEgRNA comprises a PBS comprising a sequence selected from the group consisting of sequences ACACACCA and ACACACCAT as disclosed in Table 3B. In some embodiments, the PEgRNA comprises an editing template comprising a sequence selected from the group consisting of SEQ Identifiers: C14899-C14909 of Table 3C. In some embodiments, the PEgRNA comprises an editing template comprising a sequence selected from the group consisting of SEQ ID NOs: 18925-18935 of Table 3C. In some embodiments, the prime editing composition further comprises a ngRNA that comprises a spacer comprising a sequence selected from the group consisting of SEQ Identifiers: D1671, D1695-D1698, D1708-D1721, D1796, D1804-D1809, D1819, and D1820 of Table 3D. In some embodiments, the prime editing composition further comprises a ngRNA that comprises a spacer comprising a sequence selected from the group consisting of SEQ ID NOs. 21304, 21328-21331, 21341-21354, 21429, 21437-21442, 21452, and 21453 of Table 3D.

In some embodiments, the PEgRNA comprises a spacer comprising a sequence selected from the group consisting of SEQ Identifiers: A810-A813 of Table 3A. In some embodiments, the PEgRNA comprises a spacer comprising a sequence as set forth in SEQ ID NOs. 1158-1161 of Table 3A. In some embodiments, the PEgRNA comprises a PBS comprising a sequence selected from the group consisting of SEQ Identifiers: B3375-B3383 of Table 3B. In some embodiments, the PEgRNA comprises a PBS comprising a sequence selected from the group consisting of SEQ ID NOs. 3838-3844 of Table 3B. In some embodiments, the PEgRNA comprises a PBS comprising a sequence selected from the group consisting of sequences CACACCAT and CACACCATC as disclosed in Table 3B. In some embodiments, the PEgRNA comprises an editing template comprising a sequence selected from the group consisting of SEQ Identifiers: C14910-C14919 of Table 3C. In some embodiments, the PEgRNA comprises an editing template comprising a sequence selected from the group consisting of SEQ ID NOs: 18936-18945 of Table 3C. In some embodiments, the prime editing composition further comprises a ngRNA that comprises a spacer comprising a sequence selected from the group consisting of SEQ Identifiers: D1665, D1668, D1671, D1672, D1683, D1684, D1685, D1686, D1689, D1690, D1691, D1692, D1695, D1696, D1697, D1698, D1708, D1709, D1710, D1711, D1712, D1713, D1714, D1715, D1716, D1718, D1719, D1720, D1721, D1796, D1804, D1805, D1806, D1807, D1808, D1809, D1819, D1820, D1832, D1833, and D1834 of Table 3D. In some embodiments, the prime editing composition further comprises a ngRNA that comprises a spacer comprising a sequence selected from the group consisting of SEQ ID NOs. 21298, 21301, 21304, 21305, 21316-21319, 21322-21325, 21328, 21329, 21330, 21331, 21341-21349, 21351-21354, 21429, 21437-21442, 21452, 21453, 21464, 21465, 21466, 21467 of Table 3D.

In some embodiments, the PEgRNA comprises a spacer comprising a sequence selected from the group consisting of SEQ Identifiers: A814-A816 of Table 3A. In some embodiments, the PEgRNA comprises a spacer comprising a sequence as set forth in SEQ ID NOs. 1162-1164 of Table 3A. In some embodiments, the PEgRNA comprises a PBS comprising a sequence selected from the group consisting of SEQ Identifiers: B3384-B3392 of Table 3B. In some embodiments, the PEgRNA comprises a PBS comprising a sequence selected from the group consisting of SEQ ID NOs. 3845-3851 of Table 3B. In some embodiments, the PEgRNA comprises a PBS comprising a sequence selected from the group consisting of sequences TGAGCAAA and TGAGCAAAA as disclosed in Table 3B. In some embodiments, the PEgRNA comprises an editing template comprising a sequence selected from the group consisting of SEQ Identifiers: C14920-C14930 of Table 3C. In some embodiments, the PEgRNA comprises an editing template comprising a sequence selected from the group consisting of SEQ ID NOs: 18946-18956 of Table 3C. In some embodiments, the prime editing composition further comprises a ngRNA that comprises a spacer comprising a sequence selected from the group consisting of SEQ Identifiers: D1761, D1764, D1779, D1780, D1788, and D1789 of Table 3D. In some embodiments, the prime editing composition further comprises a ngRNA that comprises a spacer comprising a sequence selected from the group consisting of SEQ ID NOs. 21394, 21397, 21412, 21413, 21421, and 21422 of Table 3D.

In some embodiments, the PEgRNA comprises a spacer comprising the sequence of SEQ Identifier: A817 of Table 3A. In some embodiments, the PEgRNA comprises a spacer comprising a sequence as set forth in SEQ ID NO. 1165 of Table 3A. In some embodiments, the PEgRNA comprises a PBS comprising a sequence selected from the group consisting of SEQ Identifiers: B3393-B3401 of Table 3B. In some embodiments, the PEgRNA comprises a PBS comprising a sequence selected from the group consisting of SEQ ID NOs. 3852-3858 of Table 3B. In some embodiments, the PEgRNA comprises a PBS comprising a sequence selected from the group consisting of sequences GAGCAAAA and GAGCAAAAG as disclosed in Table 3B. In some embodiments, the PEgRNA comprises an editing template comprising a sequence selected from the group consisting of SEQ Identifiers: C14931-C14940 of Table 3C. In some embodiments, the PEgRNA comprises an editing template comprising a sequence selected from the group consisting of SEQ ID NOs: 18957-18966 of Table 3C. In some embodiments, the prime editing composition further comprises a ngRNA that comprises a spacer comprising a sequence selected from the group consisting of SEQ Identifiers: D1645-D1650, and D1810-D1818 of Table 3D. In some embodiments, the prime editing composition further comprises a ngRNA that comprises a spacer comprising a sequence selected from the group consisting of SEQ ID NOs. 21278-21283 and 21443-21451 of Table 3D.

In some embodiments, the PEgRNA comprises a spacer comprising the sequence of SEQ Identifier: A818 of Table 3A. In some embodiments, the PEgRNA comprises a spacer comprising a sequence as set forth in SEQ ID NO. 1166 of Table 3A. In some embodiments, the PEgRNA comprises a PBS comprising a sequence selected from the group consisting of SEQ Identifiers: B3402-B3410 of Table 3B. In some embodiments, the PEgRNA comprises a PBS comprising a sequence selected from the group consisting of SEQ ID NOs. 3859-3865 of Table 3B. In some embodiments, the PEgRNA comprises a PBS comprising a sequence selected from the group consisting of sequences TCGATCTG and TCGATCTGG as disclosed in Table 3B. In some embodiments, the PEgRNA comprises an editing template comprising a sequence selected from the group consisting of SEQ Identifiers: C14941-C14943 of Table 3C. In some embodiments, the PEgRNA comprises an editing template comprising a sequence selected from the group consisting of SEQ ID NOs: 18967-18969 of Table 3C. In some embodiments, the prime editing composition further comprises a ngRNA that comprises a spacer comprising a sequence selected from the group consisting of SEQ Identifiers: D1671, D1708-D1721, D1796, and D1804-1809 of Table 3D. In some embodiments, the prime editing composition further comprises a ngRNA that comprises a spacer comprising a sequence selected from the group consisting of SEQ ID NOs. 21304, 21341-21354, 21429, and 21437-21442 of Table 3D.

In some embodiments, the PEgRNA comprises a spacer comprising a sequence selected from the group consisting of SEQ Identifiers: A819-A820 of Table 3A. In some embodiments, the PEgRNA comprises a spacer comprising a sequence as set forth in SEQ ID NOs. 1167-1168 of Table 3A. In some embodiments, the PEgRNA comprises a PBS comprising a sequence selected from the group consisting of SEQ Identifiers: B3411-B3419 of Table 3B. In some embodiments, the PEgRNA comprises a PBS comprising a sequence selected from the group consisting of SEQ ID NOs. 3866-3872 of Table 3B. In some embodiments, the PEgRNA comprises a PBS comprising a sequence selected from the group consisting of sequences CAAAGTTA and CAAAGTTAT as disclosed in Table 3B. In some embodiments, the PEgRNA comprises an editing template comprising a sequence selected from the group consisting of SEQ Identifiers: C14944-C15005 of Table 3C. In some embodiments, the PEgRNA comprises an editing template comprising a sequence selected from the group consisting of SEQ ID NOs: 18970-19031 of Table 3C. In some embodiments, the prime editing composition further comprises a ngRNA that comprises a spacer comprising a sequence selected from the group consisting of SEQ Identifiers: D1668, D1669, D1679, D1680, D1729, D1731, D1802, D1803, D1835-D1839, and D1842-D1846 of Table 3D. In some embodiments, the prime editing composition further comprises a ngRNA that comprises a spacer comprising a sequence selected from the group consisting of SEQ ID NOs. 21301, 21302, 21312, 21313, 21362, 21364, 21435, 21436, 21468-21472, and 21475-21479 of Table 3D.

In some embodiments, the PEgRNA comprises a spacer comprising the sequence of SEQ Identifier A821 of Table 3A. In some embodiments, the PEgRNA comprises a spacer comprising a sequence as set forth in SEQ ID NO. 1169 of Table 3A. In some embodiments, the PEgRNA comprises a PBS comprising a sequence selected from the group consisting of SEQ Identifiers: B3420-B3428 of Table 3B. In some embodiments, the PEgRNA comprises a PBS comprising a sequence selected from the group consisting of SEQ ID NOs. 3873-3879 of Table 3B. In some embodiments, the PEgRNA comprises a PBS comprising a sequence selected from the group consisting of sequences GCAAAGTT and GCAAAGTTA as disclosed in Table 3B. In some embodiments, the PEgRNA comprises an editing template comprising a sequence selected from the group consisting of SEQ Identifiers: C15006-C15036 of Table 3C. In some embodiments, the PEgRNA comprises an editing template comprising a sequence selected from the group consisting of SEQ ID NOs: 19032-19062 of Table 3C. In some embodiments, the prime editing composition further comprises a ngRNA that comprises a spacer comprising a sequence selected from the group consisting of SEQ Identifiers: D1714-D1721, D1796, D1804-D1809, and D1820-D1823 of Table 3D. In some embodiments, the prime editing composition further comprises a ngRNA that comprises a spacer comprising a sequence selected from the group consisting of SEQ ID NOs. 21347-21354, 21429, 21437-21442, and 21453-21456 of Table 3D.

In some embodiments, the PEgRNA comprises a spacer comprising the sequence of SEQ Identifier A822 of Table 3A. In some embodiments, the PEgRNA comprises a spacer comprising a sequence as set forth in SEQ ID NO. 1170 of Table 3A. In some embodiments, the PEgRNA comprises a PBS comprising a sequence selected from the group consisting of SEQ Identifiers: B3429-B3437 of Table 3B. In some embodiments, the PEgRNA comprises a PBS comprising a sequence selected from the group consisting of SEQ ID NOs. 3880-3886 of Table 3B. In some embodiments, the PEgRNA comprises a PBS comprising a sequence selected from the group consisting of sequences TTATTGAA and TTATTGAAT as disclosed in Table 3B. In some embodiments, the PEgRNA comprises an editing template comprising a sequence selected from the group consisting of SEQ Identifiers: C15037-C15062 of Table 3C. In some embodiments, the PEgRNA comprises an editing template comprising a sequence selected from the group consisting of SEQ ID NOs: 19063-19088 of Table 3C. In some embodiments, the prime editing composition further comprises a ngRNA that comprises a spacer comprising a sequence selected from the group consisting of SEQ Identifiers: D1712-D1721, D1796, D1804-D1809, D1820, D1821, and D1822 of Table 3D. In some embodiments, the prime editing composition further comprises a ngRNA that comprises a spacer comprising a sequence selected from the group consisting of SEQ ID NOs. 21345-21354, 21429, 21437-21442, and 21453-21455 of Table 3D.

In some embodiments, the PEgRNA comprises a spacer comprising a sequence selected from the group consisting of SEQ Identifiers: A823-A824 of Table 3A. In some embodiments, the PEgRNA comprises a spacer comprising a sequence as set forth in SEQ ID NOs. 1171-1172 of Table 3A. In some embodiments, the PEgRNA comprises a PBS comprising a sequence selected from the group consisting of SEQ Identifiers: B3438-B3446 of Table 3B. In some embodiments, the PEgRNA comprises a PBS comprising a sequence selected from the group consisting of SEQ ID NOs. 3887-3893 of Table 3B. In some embodiments, the PEgRNA comprises a PBS comprising a sequence selected from the group consisting of sequences TTGCAAAG and TTGCAAAGT as disclosed in Table 3B. In some embodiments, the PEgRNA comprises an editing template comprising a sequence selected from the group consisting of SEQ Identifiers: C15063-C15093 of Table 3C. In some embodiments, the PEgRNA comprises an editing template comprising a sequence selected from the group consisting of SEQ ID NOs: 19089-19119 of Table 3C. In some embodiments, the prime editing composition further comprises a ngRNA that comprises a spacer comprising a sequence selected from the group consisting of SEQ Identifiers: D1714-D1721, D1725, D1726, D1727, D1796, D1804-D1809, D1820-D1823, and D1828-D1831 of Table 3D. In some embodiments, the prime editing composition further comprises a ngRNA that comprises a spacer comprising a sequence selected from the group consisting of SEQ ID NOs. 21347-21354, 21358-21360, 21429, 21437-21442, 21453-21456, and 21461-21464 of Table 3D.

In some embodiments, the PEgRNA comprises a spacer comprising the sequence of SEQ Identifier: A825 of Table 3A. In some embodiments, the PEgRNA comprises a spacer comprising a sequence as set forth in SEQ ID NO. 1173 of Table 3A. In some embodiments, the PEgRNA comprises a PBS comprising a sequence selected from the group consisting of SEQ Identifiers: B3447-B3455 of Table 3B. In some embodiments, the PEgRNA comprises a PBS comprising a sequence selected from the group consisting of SEQ ID NOs. 3894-3900 of Table 3B. In some embodiments, the PEgRNA comprises a PBS comprising a sequence selected from the group consisting of sequences GGACTTAG and GGACTTAGC as disclosed in Table 3B. In some embodiments, the PEgRNA comprises an editing template comprising a sequence selected from the group consisting of SEQ Identifiers: C15094-C15095 of Table 3C. In some embodiments, the PEgRNA comprises an editing template comprising a sequence selected from the group consisting of SEQ ID NOs: 19120-19121 of Table 3C. In some embodiments, the prime editing composition further comprises a ngRNA that comprises a spacer comprising a sequence selected from the group consisting of SEQ Identifiers: D1647-D1650, and D1810-D1818 of Table 3D. In some embodiments, the prime editing composition further comprises a ngRNA that comprises a spacer comprising a sequence selected from the group consisting of SEQ ID NOs. 21280-21283 and 21443-21451 of Table 3D.

In some embodiments, the editing target sequence is in exon 24 of the CFTR gene.

In some embodiments, the editing target sequence comprises a mutation as compared to a wild type CFTR gene.

In some embodiments, the editing target sequence or the complementary sequence on the CFTR gene thereof comprises a mutation that encodes an amino acid substitution N1303K. K1302*, N1303H, D1305E, and/or N1303I relative to a wild type CFTR polypeptide set forth in SEQ ID NO: 19, wherein * refers to a non-sense mutation, and wherein the editing template encodes an intended nucleotide edit that corrects the mutation. In some embodiments, the editing target sequence or the complementary sequence on the CFTR gene thereof comprises a C→G mutation (c.3909C>G) at position 117,652,877, a A→T mutation (c.3904A>T) at position 117,652,872, a A→C mutation (c.3907A>C) at position 117,652,875, a duplication of a A (c.3908dupA) at position 117,652,876, a A→T mutation (c.3908A>T) at position 117,652,876, and/or a deletion of A (c.3908delA) at position 117,652,876, a deletion of CTTGGA and an insertion of TGT (c.3909_3914delCTTGGAinsTGT) at position 117,652,877, a T→A mutation (c.3915T>A) at position 117,652,883, and/or a duplication of a CC (c.3917_3918dupCC) at position 117,652,886 of human chromosome 7 compared to a wild type CFTR gene. In some embodiments, the editing template comprises an intended nucleotide edit compared to the target CFTR gene, wherein incorporation of the intended nucleotide edit encoded by the editing template corrects the mutation. In some embodiments, incorporation of the one or more intended nucleotide edits results in a CFTR gene that encodes a functional CFTR polypeptide.

In some embodiments, the editing template comprises a wild type CFTR gene sequence. Accordingly, in some embodiments, the newly synthesized single stranded DNA encoded by the editing template is capable of correcting the c.3909C>G mutation, the c.3904A>T mutation, the c.3907A>C mutation, the c.3908dupA mutation, the c.3908A>T mutation, the c.3908delA mutation, the c.3909_3914delCTTGGAinsTGT mutation, the c.3915T>A mutation, and/or the c.3917_3918dupCC mutation in the target CFTR gene. In some embodiments, the newly synthesized single stranded DNA encoded by the editing template is capable of correcting all of the c.3909C>G mutation, the c.3904A>T mutation, the c.3907A>C mutation, the c.3908dupA mutation, the c.3908A>T mutation, the c.3908delA mutation, the c.3909_3914delCTTGGAinsTGT mutation, the c.3915T>A mutation, and the c.3917_3918dupCC mutation in the target CFTR gene. In some embodiments, two or more of the c.3909C>G mutation, the c.3904A>T mutation, the c.3907A>C mutation, the c.3908dupA mutation, the c.3908A>T mutation, the c.3908delA mutation, the c.3909_3914delCTTGGAinsTGT mutation, the c.3915T>A mutation, and the c.3917_3918dupCC mutation are located on the same CFTR gene. In some embodiments, the newly synthesized single stranded DNA encoded by the editing template is capable of correcting the c.3909C>G mutation, the c.3904A>T mutation, the c.3907A>C mutation, the c.3908dupA mutation, the c.3908A>T mutation, the c.3908delA mutation, the c.3909_3914delCTTGGAinsTGT mutation, the c.3915T>A mutation, and the c.3917_3918dupCC mutation that are located on different CFTR genes. For example, in some embodiments, a subject may comprise two copies of CFTR genes, each comprising a different mutation selected from the group consisting of the c.3909C>G mutation, the c.3904A>T mutation, the c.3907A>C mutation, the c.3908dupA mutation, the c.3908A>T mutation, the c.3908delA mutation, the c.3909_3914delCTTGGAinsTGT mutation, the c.3915T>A mutation, and the c.3917_3918dupCC mutation, all of which may be corrected by an editing template of a single PEgRNA. In some embodiments, the editing template is capable of correcting one or more, or all of the c.3909C>G mutation, the c.3904A>T mutation, the c.3907A>C mutation, the c.3908dupA mutation, the c.3908A>T mutation, the c.3908delA mutation, the c.3909_3914delCTTGGAinsTGT mutation, the c.3915T>A mutation, and the c.3917_3918dupCC mutation that are in different subjects, wherein each subject may have one or more of the mutations.

In some embodiments, the PEgRNA comprises a spacer comprising a sequence selected from the group consisting of SEQ Identifiers: A826-A827 of Table 3A. In some embodiments, the PEgRNA comprises a spacer comprising a sequence as set forth in SEQ ID NOs. 1174-1175 of Table 3A. In some embodiments, the PEgRNA comprises a PBS comprising a sequence selected from the group consisting of SEQ Identifiers: B3456-B3464 of Table 3B. In some embodiments, the PEgRNA comprises a PBS comprising a sequence selected from the group consisting of SEQ ID NOs. 3901-3907 of Table 3B. In some embodiments, the PEgRNA comprises a PBS comprising a sequence selected from the group consisting of sequences AAAATAAA and AAAATAAAT as disclosed in Table 3B. In some embodiments, the PEgRNA comprises an editing template comprising a sequence selected from the group consisting of SEQ Identifiers: C15096-C15113 of Table 3C. In some embodiments, the PEgRNA comprises an editing template comprising a sequence selected from the group consisting of SEQ ID NOs: 19122-19139 of Table 3C. In some embodiments, the prime editing composition further comprises a ngRNA that comprises a spacer comprising a sequence selected from the group consisting of SEQ Identifiers: D1853-D1864 of Table 3D. In some embodiments, the prime editing composition further comprises a ngRNA that comprises a spacer comprising a sequence selected from the group consisting of SEQ ID NOs. 21486-21497 of Table 3D.

In some embodiments, the PEgRNA comprises a spacer comprising a sequence selected from the group consisting of SEQ Identifiers: A828-A830 of Table 3A. In some embodiments, the PEgRNA comprises a spacer comprising a sequence as set forth in SEQ ID NOs. 1176-1178 of Table 3A. In some embodiments, the PEgRNA comprises a PBS comprising a sequence selected from the group consisting of SEQ Identifiers: B3465-B3473 of Table 3B. In some embodiments, the PEgRNA comprises a PBS comprising a sequence selected from the group consisting of SEQ ID NOs. 3908-3914 of Table 3B. In some embodiments, the PEgRNA comprises a PBS comprising a sequence selected from the group consisting of sequences AAATAAAT and AAATAAATA as disclosed in Table 3B. In some embodiments, the PEgRNA comprises an editing template comprising a sequence selected from the group consisting of SEQ Identifiers: C15114-C15130 of Table 3C. In some embodiments, the PEgRNA comprises an editing template comprising a sequence selected from the group consisting of SEQ ID NOs: 19140-19156 of Table 3C. In some embodiments, the prime editing composition further comprises a ngRNA that comprises a spacer comprising a sequence selected from the group consisting of SEQ Identifiers: D1865-D1890 of Table 3D. In some embodiments, the prime editing composition further comprises a ngRNA that comprises a spacer comprising a sequence selected from the group consisting of SEQ ID NOs. 21498-21523 of Table 3D.

In some embodiments, the PEgRNA comprises a spacer comprising the sequence of SEQ Identifier A831 of Table 3A. In some embodiments, the PEgRNA comprises a spacer comprising a sequence as set forth in SEQ ID NO. 1179 of Table 3A. In some embodiments, the PEgRNA comprises a PBS comprising a sequence selected from the group consisting of SEQ Identifiers: B3474-B3482 of Table 3B. In some embodiments, the PEgRNA comprises a PBS comprising a sequence selected from the group consisting of SEQ ID NOs. 3915-3921 of Table 3B. In some embodiments, the PEgRNA comprises a PBS comprising a sequence selected from the group consisting of sequences GAGTGATC and GAGTGATCA as disclosed in Table 3B. In some embodiments, the PEgRNA comprises an editing template comprising a sequence selected from the group consisting of SEQ Identifiers: C15131-C15149 of Table 3C. In some embodiments, the PEgRNA comprises an editing template comprising a sequence selected from the group consisting of SEQ ID NOs: 19157-19175 of Table 3C. In some embodiments, the prime editing composition further comprises a ngRNA that comprises a spacer comprising a sequence selected from the group consisting of SEQ Identifiers: D1865-D1890 of Table 3D. In some embodiments, the prime editing composition further comprises a ngRNA that comprises a spacer comprising a sequence selected from the group consisting of SEQ ID NOs. 21498-21523 of Table 3D.

In some embodiments, the PEgRNA comprises a spacer comprising a sequence selected from the group consisting of SEQ Identifiers: A832-A833 of Table 3A. In some embodiments, the PEgRNA comprises a spacer comprising a sequence as set forth in SEQ ID NOs. 1180-1181 of Table 3A. In some embodiments, the PEgRNA comprises a PBS comprising a sequence selected from the group consisting of SEQ Identifiers: B3483-B3491 of Table 3B. In some embodiments, the PEgRNA comprises a PBS comprising a sequence selected from the group consisting of SEQ ID NOs. 3922-3928 of Table 3B. In some embodiments, the PEgRNA comprises a PBS comprising a sequence selected from the group consisting of sequences TGAACAGT and TGAACAGTG as disclosed in Table 3B. In some embodiments, the PEgRNA comprises an editing template comprising a sequence selected from the group consisting of SEQ Identifiers: C15150-C15177 of Table 3C. In some embodiments, the PEgRNA comprises an editing template comprising a sequence selected from the group consisting of SEQ ID NOs: 19176-19203 of Table 3C. In some embodiments, the prime editing composition further comprises a ngRNA that comprises a spacer comprising a sequence selected from the group consisting of SEQ Identifiers: D1865-D1888, and D1891-D1906 of Table 3D. In some embodiments, the prime editing composition further comprises a ngRNA that comprises a spacer comprising a sequence selected from the group consisting of SEQ ID NOs. 21498-21521, and 21524-21539 of Table 3D.

In some embodiments, the PEgRNA comprises a spacer comprising a sequence selected from the group consisting of SEQ Identifiers: A834-A836 of Table 3A. In some embodiments, the PEgRNA comprises a spacer comprising a sequence as set forth in SEQ ID NOs. 1182-1184 of Table 3A. In some embodiments, the PEgRNA comprises a PBS comprising a sequence selected from the group consisting of SEQ Identifiers: B3492-B3500 of Table 3B. In some embodiments, the PEgRNA comprises a PBS comprising a sequence selected from the group consisting of SEQ ID NOs. 3929-3935 of Table 3B. In some embodiments, the PEgRNA comprises a PBS comprising a sequence selected from the group consisting of sequences TTTTCTAA and TTTTCTAAA as disclosed in Table 3B. In some embodiments, the PEgRNA comprises an editing template comprising a sequence selected from the group consisting of SEQ Identifiers: C15178-C15208 of Table 3C. In some embodiments, the PEgRNA comprises an editing template comprising a sequence selected from the group consisting of SEQ ID NOs: 19204-19234 of Table 3C. In some embodiments, the prime editing composition further comprises a ngRNA that comprises a spacer comprising a sequence selected from the group consisting of SEQ Identifiers: D1907-D1909 of Table 3D. In some embodiments, the prime editing composition further comprises a ngRNA that comprises a spacer comprising a sequence selected from the group consisting of SEQ ID NOs. 21540-21542 of Table 3D.

In some embodiments, the PEgRNA comprises a spacer comprising a sequence selected from the group consisting of SEQ Identifiers: A837-A838 of Table 3A. In some embodiments, the PEgRNA comprises a spacer comprising a sequence as set forth in SEQ ID NOs. 1185-1186 of Table 3A. In some embodiments, the PEgRNA comprises a PBS comprising a sequence selected from the group consisting of SEQ Identifiers: B3501-B3509 of Table 3B. In some embodiments, the PEgRNA comprises a PBS comprising a sequence selected from the group consisting of SEQ ID NOs. 3936-3942 of Table 3B. In some embodiments, the PEgRNA comprises a PBS comprising a sequence selected from the group consisting of sequences AAAAATAA and AAAAATAAA as disclosed in Table 3B. In some embodiments, the PEgRNA comprises an editing template comprising a sequence selected from the group consisting of SEQ Identifiers: C15209-C15227 of Table 3C. In some embodiments, the PEgRNA comprises an editing template comprising a sequence selected from the group consisting of SEQ ID NOs: 19235-19253 of Table 3C. In some embodiments, the prime editing composition further comprises a ngRNA that comprises a spacer comprising a sequence selected from the group consisting of SEQ Identifiers: D1853-D1864, and D1910-D1916 of Table 3D. In some embodiments, the prime editing composition further comprises a ngRNA that comprises a spacer comprising a sequence selected from the group consisting of SEQ ID NOs. 21486-21497 and 21543-21549 of Table 3D.

In some embodiments, the PEgRNA comprises a spacer comprising a sequence selected from the group consisting of SEQ Identifiers: A839-A840 of Table 3A. In some embodiments, the PEgRNA comprises a spacer comprising a sequence as set forth in SEQ ID NOs. 1187-1188 of Table 3A. In some embodiments, the PEgRNA comprises a PBS comprising a sequence selected from the group consisting of SEQ Identifiers: B3510-B3518 of Table 3B. In some embodiments, the PEgRNA comprises a PBS comprising a sequence selected from the group consisting of SEQ ID NOs. 3943-3949 of Table 3B. In some embodiments, the PEgRNA comprises a PBS comprising a sequence selected from the group consisting of sequences TTTTTTCT and TTTTTTCTA as disclosed in Table 3B. In some embodiments, the PEgRNA comprises an editing template comprising a sequence selected from the group consisting of SEQ Identifiers: C15228-C15258 of Table 3C. In some embodiments, the PEgRNA comprises an editing template comprising a sequence selected from the group consisting of SEQ ID NOs: 19254-19284 of Table 3C. In some embodiments, the PEgRNA comprises an editing template comprising a sequence selected from the group consisting of SEQ ID NOs: of Table 3C. In some embodiments, the prime editing composition further comprises a ngRNA that comprises a spacer comprising a sequence selected from the group consisting of SEQ Identifiers: D1854-D1864, and D1911-D1919 of Table 3D. In some embodiments, the prime editing composition further comprises a ngRNA that comprises a spacer comprising a sequence selected from the group consisting of SEQ ID NOs. 21487-21497 and 21544-21552 of Table 3D.

In some embodiments, the PEgRNA comprises a spacer comprising a sequence selected from the group consisting of SEQ Identifiers: A841-A842 of Table 3A. In some embodiments, the PEgRNA comprises a spacer comprising a sequence as set forth in SEQ ID NOs. 1189-1190 of Table 3A. In some embodiments, the PEgRNA comprises a PBS comprising a sequence selected from the group consisting of SEQ Identifiers: B3519-B3527 of Table 3B. In some embodiments, the PEgRNA comprises a PBS comprising a sequence selected from the group consisting of SEQ ID NOs. 3950-3956 of Table 3B. In some embodiments, the PEgRNA comprises a PBS comprising a sequence selected from the group consisting of sequences GAACAGTG and GAACAGTGG as disclosed in Table 3B. In some embodiments, the PEgRNA comprises an editing template comprising a sequence selected from the group consisting of SEQ Identifiers: C15259-C15285 of Table 3C. In some embodiments, the PEgRNA comprises an editing template comprising a sequence selected from the group consisting of SEQ ID NOs: 19285-19311 of Table 3C. In some embodiments, the prime editing composition further comprises a ngRNA that comprises a spacer comprising a sequence selected from the group consisting of SEQ Identifiers: D1865-D1888, D1902-D1906, and D1920-D1927 of Table 3D. In some embodiments, the prime editing composition further comprises a ngRNA that comprises a spacer comprising a sequence selected from the group consisting of SEQ ID NOs. 21498-21521, 21535-21539, and 21553-21560 of Table 3D.

In some embodiments, the PEgRNA comprises a spacer comprising a sequence selected from the group consisting of SEQ Identifiers: A843-A844 of Table 3A. In some embodiments, the PEgRNA comprises a spacer comprising a sequence as set forth in SEQ ID NOs. 1191-1192 of Table 3A. In some embodiments, the PEgRNA comprises a PBS comprising a sequence selected from the group consisting of SEQ Identifiers: B3528-B3536 of Table 3B. In some embodiments, the PEgRNA comprises a PBS comprising a sequence selected from the group consisting of SEQ ID NOs. 3957-3963 of Table 3B. In some embodiments, the PEgRNA comprises a PBS comprising a sequence selected from the group consisting of sequences TTTTTCTA and TTTTTCTAA as disclosed in Table 3B. In some embodiments, the PEgRNA comprises an editing template comprising a sequence selected from the group consisting of SEQ Identifiers: C15286-C15347 of Table 3C. In some embodiments, the PEgRNA comprises an editing template comprising a sequence selected from the group consisting of SEQ ID NOs: 19312-19373 of Table 3C. In some embodiments, the prime editing composition further comprises a ngRNA that comprises a spacer comprising a sequence selected from the group consisting of SEQ Identifiers: D1854-D1864, D1918, and D1919 of Table 3D. In some embodiments, the prime editing composition further comprises a ngRNA that comprises a spacer comprising a sequence selected from the group consisting of SEQ ID NOs. 21487-21497, 21551, and 21552 of Table 3D.

In some embodiments, the PEgRNA comprises a spacer comprising a sequence selected from the group consisting of SEQ Identifiers: A845-A846 of Table 3A. In some embodiments, the PEgRNA comprises a spacer comprising a sequence as set forth in SEQ ID NOs. 1193-1194 of Table 3A. In some embodiments, the PEgRNA comprises a PBS comprising a sequence selected from the group consisting of SEQ Identifiers: B3537-B3545 of Table 3B. In some embodiments, the PEgRNA comprises a PBS comprising a sequence selected from the group consisting of SEQ ID NOs. 3964-3970 of Table 3B. In some embodiments, the PEgRNA comprises a PBS comprising a sequence selected from the group consisting of sequences TGTTCCAG and TGTTCCAGA as disclosed in Table 3B. In some embodiments, the PEgRNA comprises an editing template comprising a sequence selected from the group consisting of SEQ Identifiers: C15348-C15375 of Table 3C. In some embodiments, the PEgRNA comprises an editing template comprising a sequence selected from the group consisting of SEQ ID NOs: 19374-19401 of Table 3C. In some embodiments, the prime editing composition further comprises a ngRNA that comprises a spacer comprising a sequence selected from the group consisting of SEQ Identifiers: D1854-D1864, and D1911-D1918 of Table 3D. In some embodiments, the prime editing composition further comprises a ngRNA that comprises a spacer comprising a sequence selected from the group consisting of SEQ ID NOs. 21487-21497, 21544-21551 of Table 3D.

In some embodiments, the PEgRNA comprises a spacer comprising a sequence selected from the group consisting of SEQ Identifiers: A847-A850 of Table 3A. In some embodiments, the PEgRNA comprises a spacer comprising a sequence as set forth in SEQ ID NOs. 1195-1198 of Table 3A. In some embodiments, the PEgRNA comprises a PBS comprising a sequence selected from the group consisting of SEQ Identifiers: B3546-B3554 of Table 3B. In some embodiments, the PEgRNA comprises a PBS comprising a sequence selected from the group consisting of SEQ ID NOs. 3971-3977 of Table 3B. In some embodiments, the PEgRNA comprises a PBS comprising a sequence selected from the group consisting of sequences AACAGTGG and AACAGTGGA as disclosed in Table 3B. In some embodiments, the PEgRNA comprises an editing template comprising a sequence selected from the group consisting of SEQ Identifiers: C15376-C15427 of Table 3C. In some embodiments, the PEgRNA comprises an editing template comprising a sequence selected from the group consisting of SEQ ID NOs: 19402-19453 of Table 3C. In some embodiments, the prime editing composition further comprises a ngRNA that comprises a spacer comprising a sequence selected from the group consisting of SEQ Identifiers: D1865-D1888, D1903-D1906, and D1920-D1953 of Table 3D. In some embodiments, the prime editing composition further comprises a ngRNA that comprises a spacer comprising a sequence selected from the group consisting of SEQ ID NOs. 21498-21521, 21536-21539, 21553-21586 of Table 3D.

In some embodiments, the PEgRNA comprises a spacer comprising a sequence selected from the group consisting of SEQ Identifiers: A851-A852 of Table 3A. In some embodiments, the PEgRNA comprises a spacer comprising a sequence as set forth in SEQ ID NOs. 1199-1200 of Table 3A. In some embodiments, the PEgRNA comprises a PBS comprising a sequence selected from the group consisting of SEQ Identifiers: B3555-B3563 of Table 3B. In some embodiments, the PEgRNA comprises a PBS comprising a sequence selected from the group consisting of SEQ ID NOs. 3978-3984 of Table 3B. In some embodiments, the PEgRNA comprises a PBS comprising a sequence selected from the group consisting of sequences AATATGGA and AATATGGAA as disclosed in Table 3B. In some embodiments, the PEgRNA comprises an editing template comprising a sequence selected from the group consisting of SEQ Identifiers: C15428-C15434 of Table 3C. In some embodiments, the PEgRNA comprises an editing template comprising a sequence selected from the group consisting of SEQ ID NOs: 19454-19460 of Table 3C. In some embodiments, the prime editing composition further comprises a ngRNA that comprises a spacer comprising a sequence selected from the group consisting of SEQ Identifiers: D1866-D1890, D1894-D1901, D1954, and D1955 of Table 3D. In some embodiments, the prime editing composition further comprises a ngRNA that comprises a spacer comprising a sequence selected from the group consisting of SEQ ID NOs. 21499-21523, 21527-21534, 21587, and 21588 of Table 3D.

In some embodiments, the PEgRNA comprises a spacer comprising a sequence selected from the group consisting of SEQ Identifiers: A853-A854 of Table 3A. In some embodiments, the PEgRNA comprises a spacer comprising a sequence as set forth in SEQ ID NOs. 1201-1202 of Table 3A. In some embodiments, the PEgRNA comprises a PBS comprising a sequence selected from the group consisting of SEQ Identifiers: B3564-B3572 of Table 3B. In some embodiments, the PEgRNA comprises a PBS comprising a sequence selected from the group consisting of SEQ ID NOs. 3985-3991 of Table 3B. In some embodiments, the PEgRNA comprises a PBS comprising a sequence selected from the group consisting of sequences GTTCCAGA and GTTCCAGAA as disclosed in Table 3B. In some embodiments, the PEgRNA comprises an editing template comprising a sequence selected from the group consisting of SEQ Identifiers: C15435-C15488 of Table 3C. In some embodiments, the PEgRNA comprises an editing template comprising a sequence selected from the group consisting of SEQ ID NOs: 19461-19514 of Table 3C. In some embodiments, the prime editing composition further comprises a ngRNA that comprises a spacer comprising a sequence selected from the group consisting of SEQ Identifiers: D1956-D1975 of Table 3D. In some embodiments, the prime editing composition further comprises a ngRNA that comprises a spacer comprising a sequence selected from the group consisting of SEQ ID NOs. 21589-21608 of Table 3D.

In some embodiments, the PEgRNA comprises a spacer comprising a sequence selected from the group consisting of SEQ Identifiers: A855-A856 of Table 3A. In some embodiments, the PEgRNA comprises a spacer comprising a sequence as set forth in SEQ ID NOs. 1203-1204 of Table 3A. In some embodiments, the PEgRNA comprises a PBS comprising a sequence selected from the group consisting of SEQ Identifiers: B3573-B3581 of Table 3B. In some embodiments, the PEgRNA comprises a PBS comprising a sequence selected from the group consisting of SEQ ID NOs. 3992-3998 of Table 3B. In some embodiments, the PEgRNA comprises a PBS comprising a sequence selected from the group consisting of sequences TCTAAATG and TCTAAATGT as disclosed in Table 3B. In some embodiments, the PEgRNA comprises an editing template comprising a sequence selected from the group consisting of SEQ Identifiers: C15489-C15519 of Table 3C. In some embodiments, the PEgRNA comprises an editing template comprising a sequence selected from the group consisting of SEQ ID NOs: 19515-19545 of Table 3C. In some embodiments, the prime editing composition further comprises a ngRNA that comprises a spacer comprising a sequence selected from the group consisting of SEQ Identifiers: D1956-D1963, D1965-D1973, and D1975 of Table 3D. In some embodiments, the prime editing composition further comprises a ngRNA that comprises a spacer comprising a sequence selected from the group consisting of SEQ ID NOs. 21589-21596, 21598-21606, and 21608 of Table 3D.

In some embodiments, the PEgRNA comprises a spacer comprising the sequence of SEQ Identifier: A857 of Table 3A. In some embodiments, the PEgRNA comprises a spacer comprising a sequence as set forth in SEQ ID NO. 1257 of Table 3A. In some embodiments, the PEgRNA comprises a PBS comprising a sequence selected from the group consisting of SEQ Identifiers: B3582-B3590 of Table 3B. In some embodiments, the PEgRNA comprises a PBS comprising a sequence selected from the group consisting of SEQ ID NOs. 3999-4005 of Table 3B. In some embodiments, the PEgRNA comprises a PBS comprising a sequence selected from the group consisting of sequences TTCTAAAT and TTCTAAATG as disclosed in Table 3B. In some embodiments, the PEgRNA comprises an editing template comprising a sequence selected from the group consisting of SEQ Identifiers: C15520-C15550 of Table 3C. In some embodiments, the PEgRNA comprises an editing template comprising a sequence selected from the group consisting of SEQ ID NOs: 19546-19576 of Table 3C. In some embodiments, the prime editing composition further comprises a ngRNA that comprises a spacer comprising a sequence selected from the group consisting of SEQ Identifiers: D1854-D1864, D1918, and D1919 of Table 3D. In some embodiments, the prime editing composition further comprises a ngRNA that comprises a spacer comprising a sequence selected from the group consisting of SEQ ID NOs. 21487-21497, 21551, and 21552 of Table 3D.

In some embodiments, the PEgRNA comprises a spacer comprising a sequence selected from the group consisting of SEQ Identifiers: A858-A861 of Table 3A. In some embodiments, the PEgRNA comprises a spacer comprising a sequence as set forth in SEQ ID NOs. 1206-1209 of Table 3A. In some embodiments, the PEgRNA comprises a PBS comprising a sequence selected from the group consisting of SEQ Identifiers: B3591-B3599 of Table 3B. In some embodiments, the PEgRNA comprises a PBS comprising a sequence selected from the group consisting of SEQ ID NOs. 4006-4012 of Table 3B. In some embodiments, the PEgRNA comprises a PBS comprising a sequence selected from the group consisting of sequences ACAGTGGA and ACAGTGGAG as disclosed in Table 3B. In some embodiments, the PEgRNA comprises an editing template comprising a sequence selected from the group consisting of SEQ Identifiers: C15551-C15600 of Table 3C. In some embodiments, the PEgRNA comprises an editing template comprising a sequence selected from the group consisting of SEQ ID NOs: 19577-19626 of Table 3C. In some embodiments, the prime editing composition further comprises a ngRNA that comprises a spacer comprising a sequence selected from the group consisting of SEQ Identifiers: D1880, D1881, D1929-D1935, D1938-D1944, D1948-D1952, and D1976-D1991 of Table 3D. In some embodiments, the prime editing composition further comprises a ngRNA that comprises a spacer comprising a sequence selected from the group consisting of SEQ ID NOs. 21513, 21514, 21562-21568, 21571-21577, 21581-21585, and 21609-21624 of Table 3D.

In some embodiments, the PEgRNA comprises a spacer comprising a sequence selected from the group consisting of SEQ Identifiers: A862-A863 of Table 3A. In some embodiments, the PEgRNA comprises a spacer comprising a sequence as set forth in SEQ ID NOs. 1210-1211 of Table 3A. In some embodiments, the PEgRNA comprises a PBS comprising a sequence selected from the group consisting of SEQ Identifiers: B3600-B3608 of Table 3B. In some embodiments, the PEgRNA comprises a PBS comprising a sequence selected from the group consisting of SEQ ID NOs. 4013-4019 of Table 3B. In some embodiments, the PEgRNA comprises a PBS comprising a sequence selected from the group consisting of sequences ATAGCAAA and ATAGCAAAA as disclosed in Table 3B. In some embodiments, the PEgRNA comprises an editing template comprising a sequence selected from the group consisting of SEQ Identifiers: C15601-C15604 of Table 3C. In some embodiments, the PEgRNA comprises an editing template comprising a sequence selected from the group consisting of SEQ ID NOs: 19627-19630 of Table 3C. In some embodiments, the prime editing composition further comprises a ngRNA that comprises a spacer comprising a sequence selected from the group consisting of SEQ Identifiers: D1956-D1962, D1966-D1972, D1992-D1998 of Table 3D. In some embodiments, the prime editing composition further comprises a ngRNA that comprises a spacer comprising a sequence selected from the group consisting of SEQ ID NOs. 21589-21595, 21599-21605, 21625-21631 of Table 3D.

In some embodiments, the PEgRNA comprises a spacer comprising the sequence of SEQ Identifier A864 of Table 3A. In some embodiments, the PEgRNA comprises a spacer comprising a sequence as set forth in SEQ ID NO. 1212 of Table 3A. In some embodiments, the PEgRNA comprises a PBS comprising a sequence selected from the group consisting of SEQ Identifiers: B3609-B3617 of Table 3B. In some embodiments, the PEgRNA comprises a PBS comprising a sequence selected from the group consisting of SEQ ID NOs. 4020-4026 of Table 3B. In some embodiments, the PEgRNA comprises a PBS comprising a sequence selected from the group consisting of sequences TATAGCAA and TATAGCAAA as disclosed in Table 3B. In some embodiments, the PEgRNA comprises an editing template comprising a sequence selected from the group consisting of SEQ Identifiers: C15605-C15607 of Table 3C. In some embodiments, the PEgRNA comprises an editing template comprising a sequence selected from the group consisting of SEQ ID NOs: 19631-19633 of Table 3C. In some embodiments, the prime editing composition further comprises a ngRNA that comprises a spacer comprising a sequence selected from the group consisting of SEQ Identifiers: D1853-D1863 of Table 3D. In some embodiments, the prime editing composition further comprises a ngRNA that comprises a spacer comprising a sequence selected from the group consisting of SEQ ID NOs. 21486-21496 of Table 3D.

In some embodiments, a PEgRNA (or ngRNA) comprises an additional secondary structure at the 5′ end. In some embodiments, a PEgRNA (or ngRNA) comprises an additional secondary structure at the 3′ end. In some embodiments, the secondary structure comprises a pseudoknot. In some embodiments, the secondary structure comprises a pseudoknot derived from a virus. In some embodiments, the secondary structure comprises a pseudoknot of a Moloney murine leukemia virus (M-MLV) genome (a mpknot). In some embodiments, the secondary structure comprises a quadruplex. In some embodiments, the secondary structure comprises a G-quadruplex. In some embodiments, the secondary structure comprises a P4-P6 domain of a Group I intron. In some embodiments, the secondary structure comprises a riboswitch aptamer. In some embodiments, the secondary structure comprises a riboswitch aptamer derived from a prequeosine-1 riboswitch aptamer. In some embodiments, the secondary structure comprises a modified prequeosine-1 riboswitch aptamer. In some embodiments, the secondary structure is linked to one or more other component of a PEgRNA via a linker. For example, in some embodiments, the secondary structure is at the 3′ end of the PEgRNA and is linked to the 3′ end of a PBS via a linker. In some embodiments, the secondary structure is at the 5′ end of the PEgRNA and is linked to the 5′ end of a spacer via a linker. In some embodiments, the linker is a nucleotide linker that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the linker is 5 to 10 nucleotides in length. In some embodiments, the linker is 10 to 20 nucleotides in length. In some embodiments, the linker is 15 to 25 nucleotides in length. In some embodiments, the linker is 8 nucleotides in length.

In some embodiments, the linker is designed to minimize base pairing between the linker and another component of the PEgRNA. In some embodiments, the linker is designed to minimize base pairing between the linker and the spacer. In some embodiments, the linker is designed to minimize base pairing between the linker and the PBS. In some embodiments, the linker is designed to minimize base pairing between the linker and the editing template. In some embodiments, the linker is designed to minimize base pairing between the linker and the sequence of the RNA secondary structure. In some embodiments, the linker is optimized to minimize base pairing between the linker and another component of the PEgRNA, in order of the following priority: spacer, PBS, editing template and then scaffold. In some embodiments, base paring probability is calculated using ViennaRNA 2.0 as described in Lorenz, R. et al. ViennaRNA package 2.0. Algorithms Mol. Biol. 6, incorporated by reference in its entirety herein, under standard parameters (37° C., 1 M NaCl, 0.05 M MgCl2). In some embodiments, the PEgRNA comprises a RNA secondary structure and/or a linker disclosed in Nelson et al. Engineered pegRNAs improve prime editing efficiency. Nat Biotechnol. (2021), the entirety of which is incorporated herein by reference.

In some embodiments, a PEgRNA is transcribed from a nucleotide encoding the PEgRNA, for example, a DNA plasmid encoding the PEgRNA. In some embodiments, the PEgRNA comprises a self-cleaving element. In some embodiments, the self-cleaving element improves transcription and/or processing of the PEgRNA when transcribed form the nucleotide encoding the PEgRNA. In some embodiments, the PEgRNA comprises a hairpin or an RNA quadruplex. In some embodiments, the PEgRNA comprises a self-cleaving ribozyme element, for example, a hammerhead, a pistol, a hatchet, a hairpin, a VS, a twister, or a twister sister ribozyme. In some embodiments, the PEgRNA comprises a HDV ribozyme. In some embodiments, the PEgRNA comprises a hairpin recognized by Csy4. In some embodiments, the PEgRNA comprises an ENE motif. In some embodiments, the PEgRNA comprises an element for nuclear expression (ENE) from MALATI Inc RNA. In some embodiments, the PEgRNA comprises an ENE element from Kaposi's sarcoma-associated herpesvirus (KSHV). In some embodiments, the PEgRNA comprises a 3′ box of a U1 snRNA. In some embodiments, the PEgRNA forms a circular RNA.

In some embodiments, the PEgRNA comprises a RNA secondary structure or a motif that improves binding to the DNA-RNA duple or enhances PEgRNA activity. In some embodiments, the PEgRNA comprises a sequence derived from a native nucleotide element involved in reverse transcription, e.g., initiation of retroviral transcription. In some embodiments, the PEgRNA comprises a sequence of, or derived from, a primer binding site of a substrate of a reverse transcriptase, a polypurine tract (PPT), or a kissing loop. In some embodiments, the PEgRNA comprises a dimerization motif, a kissing loop, or a GNRA tetraloop—tetraloop receptor pair that results in circularization of the PEgRNA. In some embodiments, the PEgRNA comprises a RNA secondary structure of a motif that results in physical separation of the spacer and the PBS of the PEgRNA, thereby prevents occlusion of the spacer and improves PEgRNA activity. In some embodiments, the PEgRNA comprises a secondary structure or motif, e.g., a 5′ or 3′ extension in the spacer region that form a toehold or hairpin, wherein the secondary structure or motif competes favorably against annealing between the spacer and the PBS of the PEgRNA, thereby prevents occlusion of the spacer and improves PEgRNA activity.

In some embodiments, a PEgRNA comprises a gRNA core that comprises a modified direct repeat compared to the sequence of a naturally occurring CRISPR-Cas guide RNA scaffold, for example, a Cas9 gRNA scaffold. In some embodiments, the PEgRNA comprises a “flip and extension (F+E)” gRNA core, wherein one or more base pairs in a direct repeat is modified. In some embodiments, the PEgRNA comprises a first direct repeat (the first paring element or the lower stem), wherein a Uracil is changed to a Adenine (such that in the stem region, a U-A base pair is changed to a A-U base pair). In some embodiments, the PEgRNA comprises a first direct repeat wherein the fourth U-A base pair in the stem is changed to a A-U base pair. In some embodiments, the PEgRNA comprises a first direct repeat wherein one or more U-A base pair is changed to a G-C or C-G base pair. In some embodiments, the PEgRNA comprises an extended first direct repeat.

A PEgRNA and/or an ngRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs and/or ngRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs provided in the disclosure may further comprise nucleotides added to the 5′ of the PEgRNAs. In some embodiments, the PEgRNA further comprises 1, 2, or 3 additional nucleotides added to the 5′ end. The additional nucleotides can be guanine, cytosine, adenine, or uracil. In some embodiments, the additional nucleotide at the 5′ end of the PEgRNA is a guanine or cytosine. In some embodiments, the additional nucleotides can be chemically or biologically modified.

In some embodiments, the PEgRNAs provided in the disclosure may further comprise nucleotides to the 3′ of the PEgRNAs. In some embodiments, the PEgRNA further comprises 1, 2, or 3 additional nucleotides to the 3′ end. The additional nucleotides can be guanine, cytosine, adenine, or uracil. In some embodiments, the additional nucleotides at the 3′ end of the PEgRNA is a polynucleotide comprising at least 1 uracil. In some embodiments, the additional nucleotides can be chemically or biologically modified.

In some embodiments, a PEgRNA or ngRNA is produced by transcription from a template nucleotide, for example, a template plasmid. In some embodiments, a polynucleotide encoding the PEgRNA or ngRNA is appended with one or more additional nucleotides that improves PEgRNA or ngRNA function or expression, e.g., expression from a plasmid that encodes the PEgRNA or ngRNA. In some embodiments, a polynucleotide encoding a PEgRNA or ngRNA is appended with one or more additional nucleotides at the 5′ end or at the 3′ end. In some embodiments, the polynucleotide encoding the PEgRNA or ngRNA is appended with a guanine at the 5′ end, for example, if the first nucleotide at the 5′ end of the spacer is not a guanine. In some embodiments, a polynucleotide encoding the PEgRNA or ngRNA is appended with nucleotide sequence CACC at the 5′ end. In some embodiments, the polynucleotide encoding the PEgRNA or ngRNA is appended with an additional nucleotide adenine at the 3′ end, for example, if the last nucleotide at the 3′ end of the PBS is a Thymine. In some embodiments, the polynucleotide encoding the PEgRNA or ngRNA is appended with additional nucleotide sequence TTTTTT, TTTTTTT, TTTTT, or TTTT at the 3′ end. In some embodiments, the PEgRNA or ngRNA comprises the appended nucleotides from the transcription template. In some embodiments, the PEgRNA or ngRNA further comprises one or more nucleotides at the 5′ end or the 3′ end in addition to spacer, PBS, and RTT sequences. in some embodiments, the PEgRNA or ngRNA further comprises a guanine at the 5′ end, for example, when the first nucleotide at the 5′ end of the spacer is not a guanine. In some embodiments, the PEgRNA or ngRNA further comprises nucleotide sequence CACC at the 5′ end. In some embodiments, the PEgRNA or ngRNA further comprises an adenine at the 3′ end, for example, if the last nucleotide at the 3′ end of the PBS is a thymine. In some embodiments, the PEgRNA or ngRNA further comprises nucleotide sequence UUUUUUU, UUUUUU, UUUUU, or UUUU at the 3′ end.

In some embodiments, the PEgRNAs and/or ngRNAs provided in this disclosure may have undergone a chemical or biological modifications. Modifications may be made at any position within a PEgRNA or ngRNA and may include modification to a nucleobase or to a phosphate backbone of the PEgRNA or ngRNA. In some embodiments, chemical modifications can be a structure guided modifications. In some embodiments, a chemical modification is at the 5′ end and/or the 3′ end of a PEgRNA. In some embodiments, a chemical modification is at the 5′ end and/or the 3′ end of a ngRNA. In some embodiments, a chemical modification may be within the spacer sequence, the extension arm, the editing template sequence, or the primer binding site of a PEgRNA. In some embodiments, a chemical modification may be within the spacer sequence or the gRNA core of a PEgRNA or a ngRNA comprises 1, 2, 3, 4, 5, 6, 7, 8, 9, 10 or more chemically modified nucleotides at the 3′ end. In some embodiments, a PEgRNA or ngRNA comprises 3 contiguous chemically modified nucleotides at the 3′ end. In some embodiments, a chemical modification may be within the 3′ most nucleotides of a PEgRNA or ngRNA. In some embodiments, a chemical modification may be within the 3′ most end of a PEgRNA or ngRNA. In some embodiments, a chemical modification may be within the 5′ most end of a PEgRNA or ngRNA. In some embodiments, a chemical modification may be within the 3′ most nucleotides of a PEgRNA or ngRNA. In some embodiments, a chemical modification may be within the 3′ most end of a PEgRNA or ngRNA. In some embodiments, a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, or 5 or more chemically modified nucleotides at the 3′ end. In some embodiments, a PEgRNA or ngRNA comprises 1, 2, 3, 4, or 5 more chemically modified nucleotides at the 5′ end. In some embodiments, a PEgRNA or ngRNA comprises 1, 2, or 3 or more chemically modified nucleotides at the 3′ end. In some embodiments, a PEgRNA or ngRNA comprises 1, 2, or 3 more chemically modified nucleotides at the 5′ end. In some embodiments, a PEgRNA or ngRNA comprises 1, 2, 3, 4, 5, 6, 7, 8, 9, 10 or more contiguous chemically modified nucleotides at the 3′ end. In some embodiments, a PEgRNA or ngRNA comprises 1, 2, 3, 4, 5, 6, 7, 8, 9, 10 or more contiguous chemically modified nucleotides at the 5′ end. In some embodiments, a PEgRNA or ngRNA comprises 1, 2, 3, 4, or 5 contiguous chemically modified nucleotides at the 3′ end. In some embodiments, a PEgRNA or ngRNA comprises 1, 2, 3, 4, or 5 contiguous chemically modified nucleotides at the 5′ end. In some embodiments, a PEgRNA or ngRNA comprises 1, 2, or 3 contiguous chemically modified nucleotides at the 3′ end. In some embodiments, a PEgRNA or ngRNA comprises 1, 2, or 3 contiguous chemically modified nucleotides at the 5′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, or more chemically modified nucleotides near the 3′ end. In some embodiments, a PEgRNA or ngRNA comprises 3 contiguous chemically modified nucleotides at the 3′ end. In some embodiments, a PEgRNA or ngRNA comprises 3 contiguous chemically modified nucleotides at the 5′ end. In some embodiments, a PEgRNA or ngRNA comprises 1, 2, 3, 4, 5, or more chemically modified nucleotides near the 3′ end. In some embodiments, a PEgRNA or ngRNA comprises 1, 2, 3, 4, 5, or more contiguous chemically modified nucleotides near the 3′ end. In some embodiments, a PEgRNA or ngRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA or ngRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA or ngRNA comprises one or more chemical modified nucleotides in the gRNA core. As exemplified in FIG. 3, the gRNA core of a PEgRNA may comprise one or more regions of a base paired lower stem, a base paired upper stem, where the lower stem and upper stem may be connected by a bulge comprising unpaired RNAs. The gRNA core may further comprise a nexus distal from the spacer sequence. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA or ngRNA can comprise a 2′-O-thionocarbamate-protected nucleoside phosphoramidite, a 2′-O-methyl (M), a 2′-O-methyl 3′phosphorothioate (MS), or a 2′-O-methyl 3′thioPACE (MSP), or any combination thereof. In some embodiments, a chemically modified PEgRNA and/or ngRNA can comprise a ′-O-methyl (M) RNA, a 2′-O-methyl 3′phosphorothioate (MS) RNA, a 2′-O-methyl 3′thioPACE (MSP) RNA, a 2′-F RNA, a phosphorothioate bond modification, any other chemical modifications known in the art, or any combination thereof. A chemical modification may also include, for example, the incorporation of non-nucleotide linkages or modified nucleotides into the PEgRNA and/or ngRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements, such as second strand nicking ngRNAs. Components of a prime editing composition may be combined to form a complex for prime editing, or may be kept separately, e.g., for administration purposes. In some embodiments, a prime editing composition comprises a prime editor fusion protein complexed with a PEgRNA and optionally complexed with a ngRNA. In some embodiments, the prime editing composition comprises a prime editor comprising a DNA binding domain and a DNA polymerase domain associated with each other through a PEgRNA. For example, the prime editing composition may comprise a prime editor comprising a DNA binding domain and a DNA polymerase domain linked to each other by an RNA-protein recruitment aptamer RNA sequence, which is linked to a PEgRNA. In some embodiments, a prime editing composition comprises a PEgRNA and a polynucleotide, a polynucleotide construct, or a vector that encodes a prime editor fusion protein. In some embodiments, a prime editing composition comprises a PEgRNA, a ngRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the PEgRNA of a prime editing composition is associated with the DNA binding domain, e.g., a Cas9 nickase, of the prime editor. In some embodiments, the PEgRNA of a prime editing composition complexes with the DNA binding domain of a prime editor and directs the prime editor to the target DNA.

In some embodiments, a prime editing composition comprises one or more polynucleotides that encode prime editor components and/or PEgRNA or ngRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, and (ii) a PEgRNA or a polynucleotide encoding the PEgRNA. In some embodiments, a prime editing composition comprises (i) a polynucleotide encoding a fusion protein comprising a DNA binding domain and a DNA polymerase domain, (ii) a PEgRNA or a polynucleotide encoding the PEgRNA, and (iii) an ngRNA or a polynucleotide encoding the ngRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, and (iii) a PEgRNA or a polynucleotide encoding the PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a PEgRNA or a polynucleotide encoding the PEgRNA, and (iv) an ngRNA or a polynucleotide encoding the ngRNA. In some embodiments, the polynucleotide encoding the DNA biding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N(ii) a polynucleotide encoding a C-terminal half of a prime editor fusion protein and an intein-C, (iii) a PEgRNA or a polynucleotide encoding the PEgRNA, and/or (iv) an ngRNA or a polynucleotide encoding the ngRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a PEgRNA or a polynucleotide encoding the PEgRNA, and/or (iv) a ngRNA or a polynucleotide encoding the ngRNA.

In some embodiments, a prime editing system comprises one or more polynucleotides encoding one or more prime editor polypeptides, wherein activity of the prime editing system can be temporally regulated by controlling the timing in which the vectors are delivered. For example, in some embodiments, a polynucleotide encoding the prime editor and a polynucleotide encoding a PEgRNA can be delivered simultaneously. For example, in some embodiments, a polynucleotide encoding the prime editor and a polynucleotide encoding a PEgRNA can be delivered sequentially.

In some embodiments, a polynucleotide encoding a component of a prime editing system can further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

### Pharmaceutical Compositions

Disclosed herein are pharmaceutical compositions comprising any of the prime editing composition components, for example, prime editors, fusion proteins, polynucleotides encoding prime editor polypeptides, PEgRNAs, ngRNAs, and/or prime editing complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, tale magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.)

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

### Methods of Editing

The methods and compositions disclosed herein can be used to edit a target gene of interest by prime editing.

In some embodiments, the prime editing method comprises contacting a target gene, e.g., a CFTR gene, with a PEgRNA and a prime editor (PE) polypeptide described herein. In some embodiments, the target gene is double stranded, and comprises two strands of DNA complementary to each other. In some embodiments, the contacting with a PEgRNA and the contacting with a prime editor are performed sequentially. In some embodiments, the contacting with a prime editor is performed after the contacting with a PEgRNA. In some embodiments, the contacting with a PEgRNA is performed after the contacting with a prime editor. In some embodiments, the contacting with a PEgRNA, and the contacting with a prime editor are performed simultaneously. In some embodiments, the PEgRNA and the prime editor are associated in a complex prior to contacting a target gene.

In some embodiments, contacting the target gene with the prime editing composition results in binding of the PEgRNA to a target strand of the target gene, e.g., a CFTR gene. In some embodiments, contacting the target gene with the prime editing composition results in binding of the PEgRNA to a search target sequence on the target strand of the target gene upon contacting with the PEgRNA. In some embodiments, contacting the target gene with the prime editing composition results in binding of a spacer sequence of the PEgRNA to a search target sequence with the search target sequence on the target strand of the target gene upon said contacting of the PEgRNA.

In some embodiments, contacting the target gene with the prime editing composition results in binding of the prime editor to the target gene, e.g. the target CFTR gene, upon the contacting of the PE composition with the target gene. In some embodiments, the DNA binding domain of the PE associates with the PEgRNA. In some embodiments, the PE binds the target gene, e.g. a CFTR gene, directed by the PEgRNA. Accordingly, in some embodiments, the contacting of the target gene result in binding of a DNA binding domain of a prime editor of the target CFTR gene directed by the PEgRNA.

In some embodiments, contacting the target gene with the prime editing composition results in a nick in an edit strand of the target gene, e.g., a CFTR gene by the prime editor upon contacting with the target gene, thereby generating a nicked on the edit strand of the target gene. In some embodiments, contacting the target gene with the prime editing composition results in a single-stranded DNA comprising a free 3′ end at the nick site of the edit strand of the target gene. In some embodiments, contacting the target gene with the prime editing composition results in a nick in the edit strand of the target gene by a DNA binding domain of the prime editor, thereby generating a single-stranded DNA comprising a free 3′ end at the nick site. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a part of a prime editor fusion protein or prime editing complex (in cis), or as a separate protein (in trans).

In some embodiments, contacting the target gene with the prime editing composition generates an edited single stranded DNA that is coded by the editing template of the PEgRNA by DNA polymerase mediated polymerization from the 3′ free end of the single-stranded DNA at the nick site. In some embodiments, the editing template of the PEgRNA comprises one or more intended nucleotide edits compared to endogenous sequence of the target gene, e.g., a CFTR gene. In some embodiments, the intended nucleotide edits are incorporated in the target gene, by excision of the 5′ single stranded DNA of the edit strand of the target gene generated at the nick site and DNA repair. In some embodiments, the intended nucleotide edits are incorporated in the target gene by excision of the editing target sequence and DNA repair. In some embodiments, excision of the 5′ single stranded DNA of the edit strand generated at the nick site is by a flap endonuclease. In some embodiments, the flap nuclease is FENi. In some embodiments, the method further comprises contacting the target gene with a flap endonuclease. In some embodiments, the flap endonuclease is provided as a part of a prime editor fusion protein. In some embodiments, the flap endonuclease is provided in trans.

In some embodiments, contacting the target gene with the prime editing composition generates a mismatched heteroduplex comprising the edit strand of the target gene that comprises the edited single stranded DNA, and the unedited target strand of the target gene. Without being bound by theory, the endogenous DNA repair and replication may resolve the mismatched edited DNA to incorporate the nucleotide change(s) to form the desired edited target gene.

In some embodiments, the method further comprises contacting the target gene, e.g., a CFTR gene, with a nick guide (ngRNA) disclosed herein. In some embodiments, the ngRNA comprises a spacer that binds a second search target sequence on the edit strand of the target gene. In some embodiments, the contacted ngRNA directs the PE to introduce a nick in the target strand of the target gene. In some embodiments, the nick on the target strand (non-edit strand) results in endogenous DNA repair machinery to use the edit strand to repair the non-edit strand, thereby incorporating the intended nucleotide edit in both strand of the target gene and modifying the target gene. In some embodiments, the ngRNA comprises a spacer sequence that is complementary to, and may hybridize with, the second search target sequence on the edit strand only after the intended nucleotide edit(s) are incorporated in the edit strand of the target gene.

In some embodiments, the target gene is contacted by the ngRNA, the PEgRNA, and the PE simultaneously. In some embodiments, the ngRNA, the PEgRNA, and the PE form a complex when they contact the target gene. In some embodiments, the target gene is contacted with the ngRNA, the PEgRNA, and the prime editor sequentially. In some embodiments, the target gene is contacted with the ngRNA and/or the PEgRNA after contacting the target gene with the PE. In some embodiments, the target gene is contacted with the ngRNA and/or the PEgRNA before contacting the target gene with the prime editor.

In some embodiments, the target gene, e.g., a CFTR gene, is in a cell. Accordingly, also provided herein are methods of modifying a cell.

In some embodiments, the prime editing method comprises introducing a PEgRNA, a prime editor, and/or a ngRNA into the cell that has the target gene. In some embodiments, the prime editing method comprises introducing into the cell that has the target gene with a prime editing composition comprising a PEgRNA, a prime editor polypeptide, and/or a ngRNA. In some embodiments, the PEgRNA, the prime editor polypeptide, and/or the ngRNA form a complex prior to the introduction into the cell. In some embodiments, the PEgRNA, the prime editor polypeptide, and/or the ngRNA form a complex after the introduction into the cell. The prime editors, PEgRNA and/or ngRNAs, and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The prime editors, PEgRNA and/or ngRNAs, and prime editing complexes may be introduced into the cell simultaneously or sequentially.

In some embodiments, the prime editing method comprises introducing into the cell a PEgRNA or a polynucleotide encoding the PEgRNA, a prime editor polynucleotide encoding a prime editor polypeptide, and optionally an ngRNA or a polynucleotide encoding the ngRNA. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell simultaneously. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell sequentially. In some embodiments, the method comprises introducing the polynucleotide encoding the prime editor polypeptide into the cell before introduction of the PEgRNA or the polynucleotide encoding the PEgRNA and/or the ngRNA or the polynucleotide encoding the ngRNA. In some embodiments, the polynucleotide encoding the prime editor polypeptide is introduced into and expressed in the cell before introduction of the PEgRNA or the polynucleotide encoding the PEgRNA and/or the ngRNA or the polynucleotide encoding the ngRNA into the cell. In some embodiments, the polynucleotide encoding the prime editor polypeptide is introduced into the cell after the PEgRNA or the polynucleotide encoding the PEgRNA and/or the ngRNA or the polynucleotide encoding the ngRNA are introduced into the cell. The polynucleotide encoding the prime editor polypeptide, the PEgRNA or the polynucleotide encoding the PEgRNA, and/or the ngRNA or the polynucleotide encoding the ngRNA, may be introduced into the cell by any delivery approaches described herein or any delivery approach known in the art, for example, by RNPs, LNPs, viral vectors, non-viral vectors, mRNA delivery, and physical.

In some embodiments, the polynucleotide encoding the prime editor polypeptide, the polynucleotide encoding the PEgRNA, and/or the polynucleotide encoding the ngRNA integrate into the genome of the cell after being introduced into the cell. In some embodiments, the polynucleotide encoding the prime editor polypeptide, the polynucleotide encoding the PEgRNA, and/or the polynucleotide encoding the ngRNA are introduced into the cell for transient expression. Accordingly, also provided herein are cells modified by prime editing.

In some embodiments, the cell is a human cell. A cell may be of or derived from different tissues, organs, and/or cell types. In some embodiments, the cell is a primary cell. As used herein, the term primary cell means a cell isolated from an organism, e.g., a mammal, which is grown in tissue culture (i.e., in vitro) for the first time before subdivision and transfer to a subculture. In some non-limiting examples, mammalian primary cells which can be transfected and further passaged include hepatocytes, fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells and precursors of these somatic cell types. In some embodiments, the cell is a primary cell. In some embodiments, the cell is a human primary cell. In some embodiments, the cell is a pluripotent stem cell. In some embodiments, the cell is an induced human pluripotent stem cell (iPSC). In some embodiments, the cell is a lung progenitor cell. In some embodiments, the cell is a basal cell. In some embodiments, the cell is a basal cell from the respiratory epithelium, e.g., from the bronchioles or alveoli of the lung. In some embodiments, the cell is a club cell. In some embodiments, the cell is a ciliated cell. In some embodiments, the cell is an ionocyte, e.g., pulmonary human ionocyte. In some embodiments, the cell is a human basal cell. In some embodiments, the cell is a human basal cell from the respiratory epithelium, e.g., from the bronchioles or alveoli of the lung. In some embodiments, the cell is a human club cell. In some embodiments, the cell is a human ciliated cell. In some embodiments, the cell is a human ionocyte, e.g., pulmonary human ionocyte. In some embodiments, the cell is a bronchioalveolar stem cell. In some embodiments, the cell is a human bronchioalveolar stem cell. In some embodiments, the cell is a human lung progenitor cell. In some embodiments, the cell is an epithelial cell. In some embodiments, the cell is an airway epithelial cell. In some embodiments, the cell is a human airway epithelial cell. In some embodiments, the cell is a bronchial epithelial cell. In some embodiments, the cell is a human bronchial epithelial cell. In some embodiments, the cell is a human bronchial epithelial cell induced from iPSC. In some embodiments, the cell is a pancreatic epithelial cell. In some embodiments, the cell is a pancreatic ductal epithelia cell. In some embodiments, the cell is a pancreatic acinar cell. In some embodiments, the cell is a human pancreatic epithelial cell. In some embodiments, the cell is a human pancreatic ductal epithelia cell. In some embodiments, the cell is a human pancreatic acinar cell. In some embodiments, the cell is a hepatocyte. In some embodiments, the cell is a human hepatocyte. In some embodiments, the cell is a kidney epithelial cell. In some embodiments, the cell is an intestine epithelial cell. In some embodiments, the cell is a human kidney epithelial cell. In some embodiments, the cell is a human intestine epithelial cell. In some embodiments, the cell is a fibroblast. In some embodiments, the cell is a human fibroblast. In some embodiments, the cell is a lymphocyte. In some embodiments, the cell is a human lymphocyte. In some embodiments, the cell is a tuft cell. In some embodiments, the cell is a neuroendocrine cell. In some embodiments, the cell is a goblet cell. In some embodiments, the cell is a reproductive tissue epithelial cell. In some embodiments, the cell is a sperm canal epithelial cell. In some embodiments, the cell is a human tuft cell. In some embodiments, the cell is a human neuroendocrine cell. In some embodiments, the cell is a human goblet cell. In some embodiments, the cell is a human reproductive tissue epithelial cell. In some embodiments, the cell is a human sperm canal epithelial cell. In some embodiments, the cell is derived from a human subject. In some embodiments, the cell is derived from a human subject having a disease or condition associated with a target gene, e.g., CFTR. In some embodiments, the cell is a part of an organoid, e.g., an intestinal organoid. In some embodiments, the cell is part of a human organoid. In some embodiments, the cell is in a subject, e.g., a human subject.

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, the PEgRNA or the polynucleotide encoding the PEgRNA, and/or the ngRNA or the polynucleotide encoding the ngRNA into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of one or more intended nucleotide edits in the target gene in at least one of the cells in the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in a plurality of the population of cells.

In some embodiments, the population of cells are a part of an organoid. In some embodiments, the population of cells are organoid cells. In some embodiments, the population of cells are part of an intestinal organoid. Methods of generating and examining tissue-specific organoids, e.g., intestinal organoids, are known in the art.

In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in each cell of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a CFTR gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a primary cell (as measured in a population of primary cells) relative to a suitable control.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing an airway epithelial cell, bronchial epithelial cell, a basal cell, a club cell, a ciliated cell, an ionocyte, an iPSC, a fibroblast, a lymphoblast, a reproductive tissue epithelial cell, or a sperm canal epithelial cell, relative to a corresponding control airway epithelial cell, bronchial epithelial cell, basal cell, club cell, ciliated cell, ionocyte, iPSC, fibroblast, alymphoblast, reproductive tissue epithelial cell, or sperm canal epithelial cell. In some embodiments, the bronchial epithelial cell is a human bronchial epithelial cell. In some embodiments, the cell is a fibroblast. In some embodiments, the cell is a human fibroblast. In some embodiments, the cell is a lymphocyte. In some embodiments, the cell is a human lymphocyte. In some embodiments, the cell is a basal cell. In some embodiments, the cell is a club cell. In some embodiments, the cell is a ciliated cell. In some embodiments, the cell is an ionocyte e.g., pulmonary human ionocyte. In some embodiments, the cell is a human basal cell. In some embodiments, the cell is a human basal cell from the respiratory epithelium, e.g., from the bronchioles or alveoli of the lung. In some embodiments, the cell is a human club cell. In some embodiments, the cell is a human ciliated cell. In some embodiments, the cell is a human ionocyte e.g., pulmonary human ionocyte. In some embodiments, the cell is a tuft cell. In some embodiments, the cell is a neuroendocrine cell. In some embodiments, the cell is a goblet cell. In some embodiments, the cell is a reproductive tissue epithelial cell. In some embodiments, the cell is a sperm canal epithelial cell. In some embodiments, the cell is a human tuft cell. In some embodiments, the cell is a human neuroendocrine cell. In some embodiments, the cell is a human goblet cell. In some embodiments, the cell is a human reproductive tissue epithelial cell. In some embodiments, the cell is a human sperm canal epithelial cell. In some embodiments, the cell is derived from a human subject. In some embodiments, the cell is derived from a human subject having a disease or condition associated with a target gene, e.g., CFTR. In some embodiments, the cell is a part of an organoid, e.g., an intestinal organoid. In some embodiments, the cell is part of a human organoid. In some embodiments, the cell is in a subject, e.g., a human subject.

In some embodiments, the prime editing compositions provided herein are capable of incorporating one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37(3): 224-226 (2019), which is incorporated herein in its entirety. In some embodiments, the prime editing methods disclosed herein can have an indel frequency of less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a CFTR gene within the genome of a cell) to a prime editing composition.

In some embodiments, the prime editing compositions provided herein are capable of incorporating one or more intended nucleotide edits efficiently without generating a significant proportion of indels. In some embodiments, the prime editing methods disclosed herein have an editing efficiency of at least about 1% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte e.g., pulmonary human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, or a human sperm canal epithelial cell. In some embodiments, the cell is derived from a human subject. In some embodiments, the cell is derived from a human subject having a disease or condition associated with a target gene, e.g., CFTR. In some embodiments, the cell is a part of an organoid, e.g., an intestinal organoid.

In some embodiments, the prime editing methods disclosed herein have an editing efficiency of at least about 1% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 1% and an indel frequency of less than 7.5% in a target cell, e.g., an iPSC, a human iPSC, a lung progenitor cell, a human lung progenitor cell, a basal cell, a human basal cell, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 1% and an indel frequency of less than 5% in a target cell, e.g., e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 1% and an indel frequency of less than 1% in a target cell, e.g., e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 1% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 1% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell.

In some embodiments, the prime editing methods disclosed herein have an editing efficiency of at least about 5% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 5% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 5% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 5% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 5% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 5% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell.

In some embodiments, the prime editing methods disclosed herein have an editing efficiency of at least about 7.5% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 7.5% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 7.5% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 7.5% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 7.5% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 7.5% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 7.5% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell.

In some embodiments, the prime editing methods disclosed herein have an editing efficiency of at least about 10% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 10% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 10% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 10% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 10% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 10% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 10% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 15% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 15% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 15% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 15% and an indel frequency of less than 15% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 15% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 15% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 15% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell.

In some embodiments, the prime editing methods disclosed herein have an editing efficiency of at least about 20% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 20% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 20% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 20% and an indel frequency of less than 20% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 20% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 20% and an indel frequency of less than 0.5% in a target cell, e.g., human iPSC, lung progenitor cell, bronchial epithelial cell, airway epithelial cell, a fibroblast, a lymphoblast. In some embodiments, the prime editing methods disclosed herein have an editing efficiency of at least about 20% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell.

In some embodiments, the prime editing methods disclosed herein have an editing efficiency of at least about 30% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 30% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 30% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 30% and an indel frequency of less than 30% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 30% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 30% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 30% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell.

In some embodiments, the prime editing methods disclosed herein have an editing efficiency of at least about 40% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 40% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 40% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 40% and an indel frequency of less than 40% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 40% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 40% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 40% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell.

In some embodiments, the prime editing methods disclosed herein have an editing efficiency of at least about 50% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 50% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 50% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 50% and an indel frequency of less than 50% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 50% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 50% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 50% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell.

In some embodiments, the prime editing methods disclosed herein have an editing efficiency of at least about 60% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 60% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 60% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 60% and an indel frequency of less than 60% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 60% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 60% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 60% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell.

In some embodiments, the prime editing methods disclosed herein have an editing efficiency of at least about 70% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, an intestinal organoid cell, or a human intestinal organoid cell. In some embodiments, the prime editing methods disclosed herein have an editing efficiency of at least about 70% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 70% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 70% and an indel frequency of less than 70% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 70% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 70% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 70% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell.

In some embodiments, the prime editing methods disclosed herein have an editing efficiency of at least about 80% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 80% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 80% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 80% and an indel frequency of less than 80% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 80% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 80% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 80% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell.

In some embodiments, the prime editing methods disclosed herein have an editing efficiency of at least about 90% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 90% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 90% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 90% and an indel frequency of less than 90% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 90% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 90% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 90% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell.

In some embodiments, the prime editing methods disclosed herein have an editing efficiency of at least about 95% and an indel frequency of less than 10% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 95% and an indel frequency of less than 7.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 95% and an indel frequency of less than 5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 95% and an indel frequency of less than 95% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 95% and an indel frequency of less than 1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 95% and an indel frequency of less than 0.5% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, the prime editing methods disclosed herein have an editing efficiency of at least about 95% and an indel frequency of less than 0.1% in a target cell, e.g., an iPSC, a human iPSC, a lung progenitor cell, a human lung progenitor cell, a basal cell, a human basal cell, a human basal cell from the respiratory epithelium, a club cell, a human club cell, an ionocyte, a human ionocyte, a bronchial epithelial cell, an airway epithelial cell, a human bronchial epithelial cell, a human airway epithelial cell, a fibroblast, a lymphoblast, a human fibroblast, a human lymphoblast, a lymphocyte, a human lymphocyte, a tuft cell, a neuroendocrine cell, a goblet cell, a reproductive tissue epithelial cell, a sperm canal epithelial cell, a human tuft cell, a human neuroendocrine cell, a human goblet cell, a human reproductive tissue epithelial cell, a human sperm canal epithelial cell, a intestinal organoid cell, or a human intestinal organoid cell. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a CFTR gene within the genome of a cell) to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a CFTR gene within the genome of a cell) to a prime editing composition.

In some embodiments, the prime editing composition described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

In some embodiments, the prime editing compositions (e.g., PEgRNAs and prime editors as described herein) and prime editing methods disclosed herein can be used to edit a target CFTR gene. In some embodiments, the target CFTR gene comprises a mutation compared to a wild type CFTR gene. In some embodiments, the mutation is associated with cystic fibrosis. In some embodiments, the target CFTR gene comprises an editing target sequence that contains the mutation associated with cystic fibrosis. In some embodiments, the mutation is in a coding region of the target CFTR gene. In some embodiments, the mutation is in an exon of the target CFTR gene. In some embodiments, the mutation is in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, or exon 27 of the target CFTR gene as compared to a wild type CFTR gene. In some embodiments, the mutation is in exonI1 of the CFTR gene as compared to a wild type CFTR gene. In some embodiments, the mutation is in exon 12 of the CFTR gene as compared to a wild type CFTR gene. In some embodiments, the mutation is in exon 22 of the CFTR gene as compared to a wild type CFTR gene. In some embodiments, the mutation is in exon 24 of the CFTR gene as compared to a wild type CFTR gene.

In some embodiments, the mutation is in exon 3 of the CFTR gene as compared to a wild type CFTR gene. In some embodiments, the mutation is in exon 4 of the CFTR gene as compared to a wild type CFTR gene. In some embodiments, the mutation is in exon 8 of the CFTR gene as compared to a wild type CFTR gene. In some embodiments, the mutation is in exon 10 of the CFTR gene as compared to a wild type CFTR gene. In some embodiments, the mutation is in exon 12 of the CFTR gene as compared to a wild type CFTR gene. In some embodiments, the mutation is in exon 13 of the CFTR gene as compared to a wild type CFTR gene. In some embodiments, the mutation is in exon 14 of the CFTR gene as compared to a wild type CFTR gene. In some embodiments, the mutation is in exon 20 of the CFTR gene as compared to a wild type CFTR gene. In some embodiments, the mutation is in exon 22 of the CFTR gene as compared to a wild type CFTR gene. In some embodiments, the mutation is in exon 23 of the CFTR gene as compared to a wild type CFTR gene. In some embodiments, the mutation is in exon 24 of the CFTR gene as compared to a wild type CFTR gene.

In some embodiments, the prime editing method comprises contacting a target CFTR gene with a prime editing composition comprising a prime editor, a PEgRNA, and/or a ngRNA as described herein. In some embodiments, contacting the target CFTR gene with the prime editing composition results in incorporation of one or more intended nucleotide edits in the target CFTR gene as set forth in any prime editing composition or PEgRNAs as described herein. In some embodiments, the incorporation is in a region of the target CFTR gene that corresponds to an editing target sequence in the CFTR gene. In some embodiments, the one or more intended nucleotide edits comprises a single nucleotide substitution, an insertion, a deletion, or any combination thereof, compared to the endogenous sequence of the target CFTR gene. In some embodiments, incorporation of the one or more intended nucleotide edits results in replacement of one or more mutations with the corresponding sequence that encodes a wild type CFTR polypeptide set forth in SEQ ID NO: 19. In some embodiments, incorporation of the one or more intended nucleotide edits results in replacement of the one or more mutations with the corresponding sequence in a wild type CFTR gene. In some embodiments, incorporation of the one more intended nucleotide edits results in correction of a mutation in the target CFTR gene. In some embodiments, the target CFTR gene comprises an editing template sequence that contains the mutation. In some embodiments, contacting the target CFTR gene with the prime editing composition results in incorporation of one or more intended nucleotide edits in the target CFTR gene, which corrects the mutation in the editing target sequence (or a double stranded region comprising the editing target sequence and the complementary sequence to the editing target sequence on a target strand) in the target CFTR gene.

Exemplary wild type CFTR protein:

(SEQ ID NO: 19)
MQRSPLEKASVVSKLFFSWTRPILRKGYRQRLELSDIYQIPSVDSADNLS
EKLEREWDRELASKKNPKLINALRRCFFWRFMFYGIFLYLGEVTKAVQPL
LLGRIIASYDPDNKEERSIAIYLGIGLCLLFIVRTLLLHPAIFGLHHIGM
QMRIAMFSLIYKKTLKLSSRVLDKISIGQLVSLLSNNLNKFDEGLALAHF
VWIAPLQVALLMGLIWELLQASAFCGLGFLIVLALFQAGLGRMMMKYRDQ
RAGKISERLVITSEMIENIQSVKAYCWEEAMEKMIENLRQTELKLTRKAA
YVRYFNSSAFFFSGFFVVFLSVLPYALIKGIILRKIFTTISFCIVLRMAV
TRQFPWAVQTWYDSLGAINKIQDFLQKQEYKTLEYNLTTTEVVMENVTAF
WEEGFGELFEKAKQNNNNRKTSNGDDSLFFSNFSLLGTPVLKDINFKIER
GQLLAVAGSTGAGKTSLLMVIMGELEPSEGKIKHSGRISFCSQFSWIMPG
TIKENIIFGVSYDEYRYRSVIKACQLEEDISKFAEKDNIVLGEGGITLSG
GQRARISLARAVYKDADLYLLDSPFGYLDVLTEKEIFESCVCKLMANKTR
ILVTSKMEHLKKADKILILHEGSSYFYGTFSELQNLQPDFSSKLMGCDSF
DQFSAERRNSILTETLHRFSLEGDAPVSWTETKKQSFKQTGEFGEKRKNS
ILNPINSIRKFSIVQKTPLQMNGIEEDSDEPLERRLSLVPDSEQGEAILP
RISVISTGPTLQARRRQSVLNLMTHSVNQGQNIHRKTTASTRKVSLAPQA
NLTELDIYSRRLSQETGLEISEEINEEDLKECFFDDMESIPAVTTWNTYL
RYITVHKSLIFVLIWCLVIFLAEVAASLVVLWLLGNTPLQDKGNSTHSRN
NSYAVIITSTSSYYVFYIYVGVADTLLAMGFFRGLPLVHTLITVSKILHH
KMLHSVLQAPMSTLNTLKAGGILNRFSKDIAILDDLLPLTIFDFIQLLLI
VIGAIAVVAVLQPYIFVATVPVIVAFIMLRAYFLQTSQQLKQLESEGRSP
IFTHLVTSLKGLWTLRAFGRQPYFETLFHKALNLHTANWFLYLSTLRWFQ
MRIEMIFVIFFIAVTFISILTTGEGEGRVGIILTLAMNIMSTLQWAVNSS
IDVDSLMRSVSRVFKFIDMPTEGKPTKSTKPYKNGQLSKVMIIENSHVKK
DDIWPSGGQMTVKDLTAKYTEGGNAILENISFSISPGQRVGLLGRTGSGK
STLLSAFLRLLNTEGEIQIDGVSWDSITLQQWRKAFGVIPQKVFIFSGTF
RKNLDPYEQWSDQEIWKVADEVGLRSVIEQFPGKLDFVLVDGGCVLSHGH
KQLMCLARSVLSKAKILLLDEPSAHLDPVTYQIIRRTLKQAFADCTVILC
EHRIEAMLECQQFLVIEENKVRQYDSIQKLLNERSLFRQAISPSDRVKLF
PHRNSSKCKSKPQIAALKEETEEEVQDTRL

In some embodiments, the target CFTR gene is in a target cell. Accordingly, in one aspect provided herein is a method of editing a target cell comprising a target CFTR gene that encodes a polypeptide that comprises one or more mutations relative to a wild type CFTR gene. In some embodiments, the methods of the present disclosure comprise introducing a prime editing composition comprising a PEgRNA, a prime editor polypeptide, and/or a ngRNA into the target cell that has the target CFTR gene to edit the target CFTR gene, thereby generating an edited cell.

In some embodiments, the target cell is a mammalian cell. In some embodiments, the target cell is a human cell. In some embodiments, the target cell is a primary cell. In some embodiments, the target cell is a human primary cell. In some embodiments, the target cell is a progenitor cell. In some embodiments, the target cell is a human progenitor cell. In some embodiments, the target cell is a stem cell. In some embodiments, the target cell is a human stem cell. In some embodiments, the target cell is a primary cell. In some embodiments, the target cell is a human primary cell. In some embodiments, the target cell is a pluripotent stem cell. In some embodiments, the target cell is an induced human pluripotent stem cell (iPSC). In some embodiments, the target cell is a lung progenitor cell. In some embodiments, the cell is a basal cell. In some embodiments, the cell is a club cell. In some embodiments, the cell is a ciliated cell. In some embodiments, the cell is an ionocyte, e.g., human ionocyte, e.g., pulmonary human ionocyte. In some embodiments, the cell is a human basal cell. In some embodiments, the cell is a human basal cell from the respiratory epithelium. In some embodiments, the cell is a human club cell. In some embodiments, the cell is a human ciliated cell. In some embodiments, the cell is a human ionocyte, e.g., pulmonary human ionocyte. In some embodiments, the target cell is a bronchioalveolar stem cell. In some embodiments, the target cell is a human bronchioalveolar stem cell. In some embodiments, the target cell is a human lung progenitor cell. In some embodiments, the target cell is an epithelial cell. In some embodiments, the target cell is an airway epithelial cell. In some embodiments, the target cell is a human airway epithelial cell. In some embodiments, the target cell is a bronchial epithelial cell. In some embodiments, the target cell is a human bronchial epithelial cell. In some embodiments, the target cell is a human bronchial epithelial cell induced from iPSC. In some embodiments, the target cell is a pancreatic epithelial cell. In some embodiments, the target cell is a pancreatic ductal epithelia cell. In some embodiments, the target cell is a pancreatic acinar cell. In some embodiments, the target cell is a human pancreatic epithelial cell. In some embodiments, the target cell is a human pancreatic ductal epithelia cell. In some embodiments, the target cell is a human pancreatic acinar cell. In some embodiments, the target cell is a hepatocyte. In some embodiments, the target cell is a human hepatocyte. In some embodiments, the target cell is a kidney epithelial cell. In some embodiments, the target cell is an intestine epithelial cell. In some embodiments, the target cell is a human kidney epithelial cell. In some embodiments, the target cell is a human intestine epithelial cell. In some embodiments, the cell is a tuft cell. In some embodiments, the cell is a neuroendocrine cell. In some embodiments, the cell is a goblet cell. In some embodiments, the cell is a reproductive tissue epithelial cell. In some embodiments, the cell is a sperm canal epithelial cell. In some embodiments, the cell is a human tuft cell. In some embodiments, the cell is a human neuroendocrine cell. In some embodiments, the cell is a human goblet cell. In some embodiments, the cell is a human reproductive tissue epithelial cell. In some embodiments, the cell is a human sperm canal epithelial cell.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, incorporation of the one or more intended nucleotide edits in the target CFTR gene that comprises one or more mutations restores wild type expression and function of the CFTR protein encoded by the CFTR gene. In some embodiments, the target CFTR gene encodes a M1V, A46D, E56K, P67L, R74W, G85E, E92K, P99L, D110H, D110E, R117C, R117H, R170G, G178R, E193K, P205S, L206W, V232D, R334W, R334W, 1336K, T3381, S341P, R347P, R347H, R352Q, L467P, S492F, D507, F508, V520F, G542X, S549R, S549N, G551S, G551N, G551D, A455E, S549N, R553X, A559T, R560T, R560S, R560K, A561E, Y569D, D579G, D614G, R668C, L927P, S945L, S977F, L997F, F1052V, H1054D, K1060T, L1065P, R1066C, R1066M, R1066H, A1067T, R1070Q, R1070W, F1074L, H1085R, M1101K, D1270N, D1152H, L1077P, S1235R, G1244E, S1251N, S1255P, W1282X, N1303K, G1349D, Q1411X amino acid substitution as compared to the wild type CFTR protein prior to incorporation of the one or more intended nucleotide edits, wherein X is any amino acid other than the wild type amino acid. In some embodiments, the target CFTR gene encodes a G85X, R334X, G542X, S549X, G551X, R553X, R1158X, R1162X, W1282X, N1303X amino acid substitution as compared to the wild type CFTR protein prior to incorporation of the one or more intended nucleotide edits, wherein X is any amino acid other than the wild type amino acid. In some embodiments, the target CFTR gene encodes a [G85E, R334W, G542*, S549R, S549N, G551S, G551N, G551D, R553*, D1152H, R1158*, R1162*, W1282*, or N1303K amino acid substitution as compared to the wild type CFTR protein prior to incorporation of the one or more intended nucleotide edits, wherein * refers to a non-sense mutation. In some embodiments, the target CFTR gene encodes a F508 deletion as compared to the wild type CFTR protein prior to incorporation of the one or more intended nucleotide edits. In some embodiments, expression and/or function of the CFTR protein may be measured when expressed in a target cell. In some embodiments, incorporation of the one or more intended nucleotide edits in the target CFTR gene comprising one or more mutations lead to a fold change in a level of CFTR gene expression, CFTR protein expression, or a combination thereof. In some embodiments, a change in the level of CFTR expression level can comprise a fold change of, e.g., 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or greater as compared to expression in a suitable control cell not introduced with a prime editing composition described herein. In some embodiments, incorporation of the one or more intended nucleotide edits in the target CFTR gene that comprises one or more mutations restores wild type expression of the CFTR protein by at least 10%, 20%, 30%, 40%, 50%, 60%, 70%, 80%, 90%, 95%, or 99% or more as compared to wild type expression of the CFTR protein in a suitable control cell that comprises a wild type CFTR gene.

In some embodiments, incorporation of the one or more intended nucleotide edits in the target CFTR gene that comprises one or more mutations alters folding and/or assembly of the CFTR protein encoded by the target CFTR gene. In some embodiments, incorporation of the one or more intended nucleotide edits in the target CFTR gene that comprises one or more mutations restores the biological function of the CFTR protein encoded by the target CFTR gene as compared to a wild type CFTR protein. In some embodiments, the biological function of the CFTR protein is partially or completely restored as compared to a wild type CFTR protein.

In some embodiments, a CFTR protein expression and/or biological function can be measured by a functional assay. In some embodiments, the functional assay can comprise a copper sensitivity assay, a cell viability assay, or a combination thereof. In some embodiments, protein expression can be measured using a protein assay. In some embodiments, protein expression can be measured using antibody testing. In some embodiments, an antibody can comprise anti-CFTR. In some embodiments, protein expression can be measured using ELISA, mass spectrometry, Western blot, sodium dodecyl sulfate polyacrylamide gel electrophoresis (SDS-PAGE), high performance liquid chromatography (HPLC), electrophoresis, or any combination thereof. In some embodiments, a protein assay can comprise SDS-PAGE and densitometric analysis of a Coomassie Blue-stained gel. In some embodiments, biological activity of a CFTR protein can be measured by any assay known in the art, for example, a chloride efflux assay.

### Methods of Treating Cystic Fibrosis

Cystic Fibrosis (CF) is caused by mutations in the cystic fibrosis transmembrane conductance regulator (CFTR ABCC7) gene. The CFTR protein encoded by the CFTR gene is a chloride channel comprised of five structural domains: spanning domain 1 (MSD1), nucleotide-binding domain 1 (NBD1), a regulatory (R) domain, MSD2, and NBD2. The functional domains may facilitate fluid transport and surface hydration across epithelial cells of the body's tubular organs (e.g. lungs and intestines). Mutations in the CFTR gene may cause loss of function in of the CFTR protein, resulting in disease phenotype such as inflammation and infection within the lungs and intestines.

Various disease-causing mutations in the CFTR gene have been reported, most of which are rare. The most common, F508del, is present in approximately 70% of the worldwide CF population (estimated to be 100,000). To this day, no effective cure for cystic fibrosis is available. In addition, a large number of mutations besides F508del are not responsive to existing therapies.

In some embodiments, provided herein are methods for treatment of a subject diagnosed with a disease associated with or caused by one or more pathogenic mutations. In some embodiments, provided herein are methods for treatment of a subject diagnosed with a disease associated with or caused by one or more pathogenic mutations that can be corrected by prime editing. In some embodiments, methods of treatment provided herein comprises editing one or more genes other than the gene that harbors the one or more pathogenic mutations. In some embodiments, provided herein are methods for treating Cystic Fibrosis that comprise administering to a subject a therapeutically effective amount of a prime editing composition, or a pharmaceutical composition comprising a prime editing composition as described herein. In some embodiments, administration of the prime editing composition results in incorporation of one or more intended nucleotide edits in the target gene in the subject. In some embodiments, administration of the prime editing composition results in correction of one or more pathogenic mutations, e.g., point mutations, insertions, or deletions, associated with Cystic Fibrosis in the subject. In some embodiments, administration of the prime editing composition results in correction of one or more pathogenic mutations, e.g., point mutations, insertions, or deletions in the target CFTR gene associated with cystic fibrosis in the subject. In some embodiments, the target gene comprise an editing target sequence that contains the pathogenic mutation. In some embodiments, administration of the prime editing composition results in incorporation of one or more intended nucleotide edits in the target gene that corrects the pathogenic mutation in the editing target sequence (or a double stranded region comprising the editing target sequence and the complementary sequence to the editing target sequence on a target strand) of the target gene in the subject.

In some embodiments, the method provided herein comprises administering to a subject an effective amount of a prime editing composition, for example, a PEgRNA, a prime editor, and/or a ngRNA. In some embodiments, the method comprises administering to the subject an effective amount of a prime editing composition described herein, for example, polynucleotides, vectors, or constructs that encode prime editing composition components, or RNPs, LNPs, and/or polypeptides comprising prime editing composition components. Prime editing compositions can be administered to target the CFTR gene in a subject, e.g., a human subject, suffering from, having, susceptible to, or at risk for Cystic Fibrosis. Identifying a subject in need of such treatment can be in the judgment of a subject or a health care professional and can be subjective (e.g., opinion) or objective (e.g., measurable by a test or diagnostic method). In some embodiments, the subject has Cystic Fibrosis.

In some embodiments, a population of patients each having one or more mutations in the target CFTR gene may be treated with a prime editing composition (e.g., a PEgRNA, a prime editor, and optionally an ngRNA as described herein) disclosed herein. In some embodiments, a population of patients with different mutations in the target gene can be treated with the same prime editing composition comprising a single PEgRNA, a prime editor, and optionally an ngRNA. In some embodiments, a single prime editing composition comprising a single PEgRNA and a prime editor can be used to correct one or more, or two or more, mutations in the target gene in a populations of patients, wherein one or more patients in the population have different mutations from one another. In some embodiments, the prime editing composition comprising a single pair of PEgRNA, a prime editor, and optionally an ngRNA can be used to correct 2, 3, 4, 5, 6, 7, 8, 9, 10, 15, 20, 25 or more mutations in the target gene in a population of patients, wherein one or more patients in the population have different mutations from one another. In some embodiments, the PEgRNA comprises an editing template comprising a wild type sequence of the gene.

In some embodiments, a patient with multiple mutations in the target CFTR gene can be treated with a prime editing composition (e.g., a PEgRNAs, a prime editor, and optionally an ngRNA as described herein). For example, in some embodiments, a subject may comprise two copies of the gene, each comprising one or more different mutations. In some embodiments, a patient with one or more different mutations in the target gene can be treated with a single prime editing composition comprising a PEgRNAs, a prime editor, and optionally an ngRNA.

In some embodiments, the subject has been diagnosed with Cystic Fibrosis by sequencing of a CFTR gene in the subject. In some embodiments, the subject comprises at least a copy of the CFTR gene that comprises one or more mutations compared to a wild type CFTR gene. In some embodiments, the one or more mutations result in a truncated, aberrant, or non-functional CFTR protein encoded by the CFTR gene. In some embodiments, administration of the prime editing composition described herein results in incorporation of one or more intended nucleotide edits in the CFTR gene, thereby editing the CFTR gene and treating CF. In some embodiments, incorporation of the one or more intended nucleotide edits corrects the one or more mutations to wild type nucleotides at corresponding positions in the CFTR gene. In some embodiments, incorporation of the one or more intended nucleotide edits does not correct the one or more mutations to wild type nucleotides, but allows for expression of a functional CFTR protein encoded by the CFTR gene. For example, in some embodiments, incorporation of the one or more intended nucleotide edits results in one or more codons (tri-nucleotides) that are different from a wild type codon but encode one or more amino acids same as the wild type CFTR protein. In some embodiments, incorporation of the one or more intended nucleotide edits results in one or more codons that encode one or more amino acids different from the wild type CFTR protein, but allows for expression of a functional CFTR protein.

In some embodiments, the subject comprises at least a copy of the CFTR gene that comprises two or more mutations compared to a wild type CFTR gene. In some embodiments, the two or more mutations result in a truncated, aberrant, or non-functional CFTR protein encoded by the CFTR gene. In some embodiments, administration of the prime editing composition described herein results in incorporation of one or more intended nucleotide edits in the CFTR gene, thereby correcting the two or more mutations compared to a wild type CFTR gene. In some embodiments, the two or more mutations may be edited by administering a prime editing composition comprising a single PEgRNA that results in expression of a functional CFTR protein. In some embodiments, the prime editing composition comprises at least two PEgRNAs. In some embodiments, the prime editing composition comprises a single PEgRNA.

In some embodiments, the subject comprises two copies of CTR gene, each of which comprising one or more mutations compared to a wild type CFTR gene. In some embodiments, the subject comprises same mutation(s) on both copies of the CFTR gene compared to a wild type CFTR gene. In some embodiments, the subject comprises two copies of CFTR genes, each of which comprising a different mutation compared to a wild type CFTR gene. In some embodiments, the subject comprise two copies of CFTR genes, each of which comprising two or more mutations compared to a wild type CFTR gene. In some embodiments, the subject comprises two copies of CFTR genes, each of which comprising two or more different mutations compared to a wild type CFTR gene. In some embodiments, the one or more or the two or more mutations are in a coding region of the CFTR gene. In some embodiments, the one or more or the two or more mutations result in a truncated, aberrant, or non-functional CFTR protein encoded by the CFTR gene. In some embodiments, administration of the prime editing composition described herein results in incorporation of one or more intended nucleotide edits in the CFTR gene, thereby correcting the one or more or two or more mutations on at least one copy of the CFTR gene. In some embodiments, administration of the prime editing composition described herein results in incorporation of one or more intended nucleotide edits in the CFTR gene, thereby correcting the one or more or two or more mutations on both copies of the CFTR gene. In some embodiments, incorporation of one or more intended nucleotide edits in the CFTR gene, thereby correcting the one or more or two or more mutations on at least one copy of the CFTR gene. In some embodiments, administration of the prime editing composition described herein results in incorporation of one or more intended nucleotide edits in the CFTR gene, thereby correcting the one or more or two or more mutations on both copies of the CFTR gene. In some embodiments, incorporation of the one or more intended nucleotide edits results in one or more codons that encode one or more amino acids different from the wild type CFTR protein, but allows for expression of a functional CFTR protein. In some embodiments, the prime editing composition comprises at least two PEgRNAs. In some embodiments, the prime editing composition comprises a single PEgRNA.

In some embodiments, the subject comprises at least a copy of CFTR gene that comprises a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, or exon 27 as compared to a wild type CFTR gene, wherein in administering a prime editing composition corrects the mutation compared to a wild type CFTR gene sequence. In some embodiments, the subject comprises two copies of CFTR gene, each of which comprises a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, or exon 27 as compared to a wild type CFTR gene, wherein in administering a prime editing composition corrects the mutation compared to a wild type CFTR gene sequence. In some embodiments, the prime editing composition comprises at least two PEgRNAs. In some embodiments, the prime editing composition comprises a single PEgRNA.

In some embodiments, the subject comprises at least a copy of CFTR gene that comprises one or more mutations that encode one or more of a G85E mutation, a G85V mutation, a R117L mutation, a R117H mutation, a R117P mutation, a R334W mutation, a R347C mutation, a R347P mutation, a R347L mutation, a R347H mutation, a A455V mutation, a A455E mutation, a F508 deletion, a F5081 mutation, a F508S mutation, a F508C mutation, a G542* mutation, a S549R mutation, a S549I mutation, a S549N mutation, a S549R mutation, a G551S mutation, a G551A mutation, a G551D mutation, a R553G mutation, a R553* mutation, a R553Q mutation, a K684 frameshift mutation, a D1152H mutation, a R1158* mutation, a R1162* mutation, a R1162L mutation, a R1162Q mutation, a K1177R mutation, a W1282R mutation, a W1282G mutation, a W1282* mutation, a W1282C mutation, a N1303H mutation, a N1303I mutation, and a N1303K mutation as compared to a wild type CFTR protein as set forth in SEQ ID NO: 19, wherein in administering a prime editing composition corrects the one or more mutations. In some embodiments, the subject comprises two copies of CFTR gene, wherein each copy of the CFTR gene comprises one or more mutations that encode one or more of a G85E mutation, a G85V mutation, a R117L mutation, a R117H mutation, a R117P mutation, a R334W mutation, a R347C mutation, a R347P mutation, a R347L mutation, a R347H mutation, a A455V mutation, a A455E mutation, a F508 deletion, a F508I mutation, a F508S mutation, a F508C mutation, a G542* mutation, a S549R mutation, a S549I mutation, a S549N mutation, a S549R mutation, a G551S mutation, a G551A mutation, a G551D mutation, a R553G mutation, a R553* mutation, a R553Q mutation, a K684 frameshift mutation, a D1152H mutation, a R1158* mutation, a R1162* mutation, a R1162L mutation, a R1162Q mutation, a K1177R mutation, a W1282R mutation, a W1282G mutation, a W1282* mutation, a W1282C mutation, a N1303H mutation, a N1303I mutation, and a N1303K mutation as compared to a wild type CFTR protein as set forth in SEQ ID NO: 19, wherein in administering a prime editing composition corrects the one or more mutations on both copies of the CFTR gene. In some embodiments, the prime editing composition comprises at least two PEgRNAs. In some embodiments, the prime editing composition comprises a single PEgRNA.

In some embodiments, the subject comprises at least a copy of CFTR gene that comprises two or more of a c.254G>T mutation, a c.254G>A mutation, a c.248 TT duplication, a c.252T>A-mutation, a c.259T>A mutation, a c.262_263 TT deletion, a c.263T>G mutation, a c.263T>A mutation, and a c.264_268 ATATT deletion compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from a c.254G>T mutation, a c.254G>T mutation, a c.248 TT duplication, a c.252T>A-mutation, a c.259T>A mutation, a c.262_263 TT deletion, a c.263T>G mutation, a c.263T>A mutation, and a c.264_268 ATATT deletion compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.340A>T mutation; a c.340_342 AAG deletion; a c.341_353 AGGAGGAACGCTC (SEQ ID NO: 348) deletion del; a c.343G>T mutation; a c.345_347 GGA deletion; a c.346G>C mutation; a c.346G>T mutation; a c.349C>G mutation; a c.350G>C mutation; a c.350G>T mutation; a c.350G>A mutation; a c.357 deletion of nucleotide C; and a c.358G>C mutation compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from a c.340A>T mutation; a c.340_342 AAG deletion; a c.341_353 AGGAGGAACGCTC (SEQ ID NO: 348) deletion del; a c.343G>T mutation; a c.345_347 GGA deletion; a c.346G>C mutation; a c.346G>T mutation; a c.349C>G mutation; a c.350G>C mutation; a c.350G>T mutation; a c.357 deletion of nucleotide C; and a c.358G>C mutation compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.1000C→T mutation, a c.1001G→T mutation, a c.1006_1007 insertion of nucleotide G, and a c.1007T→A mutation compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from c.1000C→T mutation, a c.1001G→T mutation, a c.1006_1007 insertion of nucleotide G, and a c.1007T→A mutation compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.1039C→T mutation, a c.1029_1030 insertion of nucleotide G, a c.1029 deletion of nucleotide C, a c.1040G→C mutation, a c.1040G→T mutation, and a c.1043T→A mutation compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from c.1039C→T mutation, a c.1029_1030 insertion of nucleotide G, a c.1029 deletion of nucleotide C, a c.1040G→C mutation, a c.1040G→T mutation, and a c.1043T→A mutation compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.1364C→T mutation, a c.1355A→C mutation, a c.1358 deletion of nucleotide T, a c.1360_1362 TTG deletion, a c.1364C→A mutation, a c.1365_1366 GG deletion, a c.1366G→T mutation, a c.1366 deletion of nucleotide G, a c.1369G→C mutation, a c.1373 deletion of nucleotide G, and a c.1373G→T mutation compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from c.1364C→T mutation, a c.1355A→C mutation, a c.1358 deletion of nucleotide T, a c.1360_1362 TTG deletion, a c.1364C→A mutation, a c.1365_1366 GG deletion, a c.1366G→T mutation, a c.1366 deletion of nucleotide G, a c.1369G→C mutation, a c.1373 deletion of nucleotide G, and a c.1373G→T mutation compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.1521_1523 T→C mutation, a c.1513A→C mutation, a c.1514 deletion of nucleotide A, a c.1516A→C mutation, a c.1517T-G mutation, a c.1518C-G mutation, a c.1519_1521 ATC deletion, a c.1516_1520 ATCAT duplication, a c.1520T-A mutation, a c.1521_1523 CTT deletion, a c.1522T→A mutation, a c.1523T-G mutation, a c.1525G→C mutation, a c.1526G→C mutation, and a c.1528 deletion of nucleotide G, and a c.1530_1531 TT deletion compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from c.1521_1523 T→C mutation, a c.1513A→C mutation, a c.1514 deletion of nucleotide A, a c.1516A→C mutation, a c.1517T-G mutation, a c.1518C-G mutation, a c.1519_1521 ATC deletion, a c.1516_1520 ATCAT duplication, a c.1520T-A mutation, a c.1521_1523 CTT deletion, a c.1522T→A mutation, a c.1523T-G mutation, a c.1525G→C mutation, a c.1526G→C mutation, a c.1528 deletion of nucleotide G, and a c.1530_1531 TT deletion compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.1616_1617 TA duplication, a c.1624G→T mutation, a c.1628A→C mutation, a c.1630G→T mutation, a c.1631G→T mutation, and a c.1634G→T mutation compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from a c.1616_1617 TA duplication, a c.1624G→T mutation, a c.1628A→C mutation, a c.1630G→T mutation, a c.1631G→T mutation, and a c.1634G→T mutation compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.1645A→C mutation, a c.1636_1641 ATCACA deletion, a c.1642_1643 CT deletion, a c.1643T→A mutation, a c.1646G→T mutation, a c.1646G→A mutation, a c.1647T→G mutation, a c.1648G→T mutation, a c.1650 deletion of nucleotide A, a c.1652G→C mutation, a c.1652 deletion of nucleotide G, a c.1654C→A mutation, c.1656 deletion of nucleotide A, chr7:a c.1657C→G mutation, c.1651G→A mutation, a c.1660_1661 insertion of nucleotide A, a c.1661C→A mutation, a c.1652G→A mutation, a c.1657C→T mutation, and a c.1658G→A mutation compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from a c.1645A→C mutation, a c.1636_1641 ATCACA deletion, a c.1642_1643 CT deletion, a c.1643T→A mutation, a c.1646G→T mutation, a c.1646G→A mutation, a c.1647T→G mutation, a c.1648G→T mutation, a c.1650 deletion of nucleotide A, a c.1652G→C mutation, a c.1652 deletion of nucleotide G, a c.1654C→A mutation, c.1656 deletion of nucleotide A, chr7:a c.1657C→G mutation, c.1651G→A mutation, a c.1660_1661 insertion of nucleotide A, a c.1661C→A mutation, a c.1652G→A mutation, a c.1657C→T mutation, and a c.1658G→A mutation compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.2052 deletion of nucleotide A, a c.2042A→T mutation, a c.2044 deletion of nucleotide A, a c.2045 duplication of nucleotide C, a c.2051_2052 deletion of nucleotides AA and insertion of nucleotide G, a c.2051_2052 AA deletion, a c.2052 duplication of nucleotide A, a c.2052 deletion of nucleotide A, a c.2053 duplication of nucleotide C, a c.2057C→A mutation, a c.2058_2061 TTTT duplication, a c.2061deletion of nucleotide T, and a c.2062A→T mutation compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from c.2052 deletion of nucleotide A, a c.2042A→T mutation, a c.2044 deletion of nucleotide A, a c.2045 duplication of nucleotide C, a c.2051_2052 deletion of nucleotides AA and insertion of nucleotide G, a c.2051_2052 AA deletion, a c.2052 duplication of nucleotide A, a c.2052 deletion of nucleotide A, a c.2053 duplication of nucleotide C, a c.2057C→A mutation, a c.2058_2061 TTTT duplication, a c.2061deletion of nucleotide T, and a c.2062A→T mutation compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.3454G→C mutation, a c.3444C→A mutation, a c.3444 deletion of nucleotide C, a c.3445 deletion of nucleotide T, a c.3458T→A mutation, and a c.3460G→T mutation compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from c.3454G→C mutation, a c.3444C→A mutation, a c.3444 deletion of nucleotide C, a c.3445 deletion of nucleotide T, a c.3458T→A mutation, and a c.3460G→T mutation compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.3472C→T mutation, a c.3477 deletion of nucleotide T, c.3484C→T mutation, a c.3483C→G mutation, a c.3485G→T mutation, a c.3486_3487 AG deletion, a c.3492 duplication of nucleotide T, a c.3494A→C mutation, and a c.3495 deletion of nucleotide G compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from c.3472C→T mutation, a c.3477 deletion of nucleotide T, c.3484C→T mutation, a c.3483C→G mutation, a c.3485G→T mutation, a c.3486_3487 AG deletion, a c.3492 duplication of nucleotide T, a c.3494A→C mutation, and a c.3495 deletion of nucleotide G compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.3530A-G mutation, a c.3528 deletion of nucleotide C, a c.3529A→T mutation, a c.3530 deletion of nucleotide A, a c.3532_3535 TCAA duplication, a c.3536_3539 CCAA deletion, and a c.3540 deletion of nucleotide A compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from c.3530A→G mutation, a c.3528 deletion of nucleotide C, a c.3529A→T mutation, a c.3530 deletion of nucleotide A, a c.3532_3535 TCAA duplication, a c.3536_3539 CCAA deletion, and a c.3540 deletion of nucleotide A compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.3844T→C mutation, a c.3844T→G mutation, a c.3846G→T mutation, a c.3848G→T mutation, c.3846G→A mutation, and a c.3855 deletion of nucleotide C compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from c.3844T→C mutation, a c.3844T-G mutation, a c.3846G→T mutation, a c.3848G→T mutation, c.3846G→A mutation, and a c.3855 deletion of nucleotide compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.3907A→C mutation, a c.3904A→T mutation, a c.3908 duplication of nucleotide A, a c.3908A→T mutation, a c.3908 deletion of nucleotide A, a c.3909C→G mutation, a c.3909_3914 deletion of nucleotides CTTGGA and insertion of nucleotides TGT, a c.3915T→A mutation, and a c.3917_3918 CC duplication compared to a wild type CFTR gene, wherein administration of a prime editing composition corrects the two or more mutations and/or results in expression of a functional CFTR protein. In some embodiments, the subject comprises two copies of CFTR gene, wherein each copy of the CFTR gene comprises a one or more different mutations selected from c.3907A→C mutation, a c.3904A→T mutation, a c.3908 duplication of nucleotide A, a c.3908A→T mutation, a c.3908 deletion of nucleotide A, a c.3909C-G mutation, a c.3909_3914 deletion of nucleotides CTTGGA and insertion of nucleotides TGT, a c.3915T→A mutation, and a c.3917_3918 CC duplication compared to a wild type CFTR gene, wherein administration of a prime editing composition corrects the two or more mutations and/or results in expression of a functional CFTR protein. In some embodiments, the prime editing composition comprises two or more PEgRNAs. In some embodiments, the prime editing composition comprises a single PEgRNA.

In some embodiments, the subject comprises at least a copy of CFTR gene that comprises a c.1521_1523 CTT deletion as compared to a wild type CFTR gene. In some embodiments, the subject comprises two copies of CFTR gene, each of which comprises a c.1521_1523 CTT deletion as compared to a wild type CFTR gene. In some embodiments, administration of a prime editing composition results in incorporation of one or more intended nucleotide edits in the CFTR gene, wherein incorporation of the one or more intended nucleotide edits results in expression of a functional CFTR protein. In some embodiments, administration of a prime editing composition results in incorporation of one or more intended nucleotide edits in the CFTR gene, wherein incorporation of the one or more intended nucleotide edits corrects the c.1521_1523 CTT deletion on one or both copies of the CFTR gene. In some embodiments, the subject comprises two copies of CFTR gene, wherein a first copy comprises a c.1521_1523 CTT deletion as compared to a wild type CFTR gene and wherein a second copy comprises a pathogenic mutation other than a c.1521_1523 CTT deletion as compared to a wild type CFTR gene. In some embodiments, administration of a prime editing composition results in incorporation of one or more intended nucleotide edits in the CFTR gene, wherein incorporation of the one or more intended nucleotide edits results in expression of a functional CFTR protein encoded by at least one copy of the two copies of CFTR gene. In some embodiments, administration of a prime editing composition results in incorporation of one or more intended nucleotide edits in the CFTR gene, wherein incorporation of the one or more intended nucleotide edits corrects the c.1521_1523 CTT deletion and/or the pathogenic mutation other than a c.1521_1523 CTT deletion. In some embodiments, the prime editing composition comprises two or more PEgRNAs. In some embodiments, the prime editing composition comprises a single PEgRNA.

In some embodiments, the subject comprises at least a copy of CFTR gene that comprises two or more of a c.1624G>T mutation, a c.1646G>A mutation, a c.1652G>A mutation, and a c.1657C>T mutation compared to a wild type CFTR gene. In some embodiments, administration of a prime editing composition results in incorporation of one or more intended nucleotide edits in the CFTR gene, wherein incorporation of the one or more intended nucleotide edits results in expression of a functional CFTR protein. For example, in some embodiments, the CFTR gene comprises a c.1624G>T mutation, incorporation of the one or more nucleotide edits results in a A>T or A>C substitution at a position corresponding to position 117587778 of human chromosome 7, thereby replacing the G542 nonsense mutation in the CFTR protein encoded by the CFTR gene with a Cystine and allowing for expression of a functional CFTR protein. In some embodiments, administration of the prime editing composition results in incorporation of one or more intended nucleotide edits in the CFTR gene, wherein incorporation of the one or more intended nucleotide edits corrects each of the two or more mutations in the CFTR gene. In some embodiments, the subject comprises two copies of CFTR gene, wherein each of the two copies comprises a mutation selected from a c.1624G>T mutation, a c.1646G>A mutation, a c.1652G>A mutation, and a c.1657C>T mutation compared to a wild type CFTR gene. In some embodiments, each of the two copies of CFTR gene comprises a different mutation selected from a c.1624G>T mutation, a c.1646G>A mutation, a c.1652G>A mutation, and a c.1657C>T mutation compared to a wild type CFTR gene. In some embodiments, administration of a prime editing composition results in incorporation of one or more intended nucleotide edits in the CFTR gene, wherein incorporation of the one or more intended nucleotide edits results in expression of a functional CFTR protein. In some embodiments, administration of the prime editing composition results in incorporation of one or more intended nucleotide edits in the CFTR gene, wherein incorporation of the one or more intended nucleotide edits corrects each of the two or more mutations in the CFTR gene. In some embodiments, the prime editing composition comprises two or more PEgRNAs. In some embodiments, the prime editing composition comprises a single PEgRNA.

In some embodiments, the subject comprises at least a copy of CFTR gene that comprises a c.3472C>T mutation and a c.3484C>T mutation compared to a wild type CFTR gene. In some embodiments, administration of a prime editing composition results in incorporation of one or more intended nucleotide edits in the CFTR gene, wherein incorporation of the one or more intended nucleotide edits results in expression of a functional CFTR protein. In some embodiments, administration of the prime editing composition results in incorporation of one or more intended nucleotide edits in the CFTR gene, wherein incorporation of the one or more intended nucleotide edits corrects both the c.3472C>T mutation and the c.3484C>T mutation in the CFTR gene. In some embodiments, the subject comprises two copies of CFTR gene, wherein each of the two copies comprises a c.3472C>T mutation and/or a c.3484C>T mutation compared to a wild type CFTR gene. In some embodiments, each of the two copies of CFTR gene comprises a different mutation selected from a c.3472C>T mutation and a c.3484C>T mutation compared to a wild type CFTR gene. In some embodiments, administration of a prime editing composition results in incorporation of one or more intended nucleotide edits in the CFTR gene, wherein incorporation of the one or more intended nucleotide edits results in expression of a functional CFTR protein. In some embodiments, administration of the prime editing composition results in incorporation of one or more intended nucleotide edits in the CFTR gene, wherein incorporation of the one or more intended nucleotide edits corrects each of the two or more mutations in the CFTR gene. In some embodiments, the prime editing composition comprises two or more PEgRNAs. In some embodiments, the prime editing composition comprises a single PEgRNA.

In some embodiments, the subject comprises at least a copy of the CFTR gene comprising a mutation that encodes an amino acid substitution F508del relative to a wild type CFTR polypeptide set forth in SEQ ID NO: 19. In some embodiments, the subject comprises at least a copy of CFTR gene that comprises a CTT deletion at positions chr7:117559591-117559594 (GRCh38) of the CFTR gene. In some embodiments, the PEgRNA comprises a spacer comprising a sequence selected from SEQ ID NOs: A330-A334, A343-A361, and A364-A380. In some embodiments, the PEgRNA comprises a PBS comprising a sequence selected from the group consisting of SEQ ID NOs: B1377-B1403, B1413-B1520, and B1530-B1565. In some embodiments, the PEgRNA comprises an editing template comprising a sequence selected from the group consisting of SEQ ID NO: C4592-C4964 and C4976-C5414.

In some embodiments, the method comprises directly administering prime editing compositions provided herein to a subject. The prime editing compositions described herein can be delivered with in any form as described herein, e.g., as LNPs, RNPs, polynucleotide vectors such as viral vectors, or mRNAs. The prime editing compositions can be formulated with any pharmaceutically acceptable carrier described herein or known in the art for administering directly to a subject. Components of a prime editing composition or a pharmaceutical composition thereof may be administered to the subject simultaneously or sequentially. For example, in some embodiments, the method comprises administering a prime editing composition, or pharmaceutical composition thereof, comprising a complex that comprises a prime editor fusion protein and a PEgRNA and/or a ngRNA, to a subject. In some embodiments, the method comprises administering a polynucleotide or vector encoding a prime editor to a subject simultaneously with a PEgRNA and/or a ngRNA. In some embodiments, the method comprises administering a polynucleotide or vector encoding a prime editor to a subject before administration with a PEgRNA and/or a ngRNA.

Suitable routes of administrating the prime editing compositions to a subject include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the compositions described are administered intraperitoneally, intravenously, or by direct injection or direct infusion. In some embodiments, the compositions described are administered by inhalation therapy, e.g., by aerosolized or nebulized compositions. In some embodiments, the compositions described are administered by direct injection or infusion into an affected organ or tissue of a subject, e.g., the lungs or pancreas. In some embodiments, the compositions described herein are administered to a subject by injection, by means of a catheter, by means of a suppository, or by means of an implant.

In some embodiments, the method comprises administering cells edited with a prime editing composition described herein to a subject. In some embodiments, the cells are allogeneic. In some embodiments, allogeneic cells are or have been contacted ex vivo with a prime editing composition or pharmaceutical composition thereof and are introduced into a human subject in need thereof. In some embodiments, the cells are autologous to the subject. In some embodiments, cells are removed from a subject and contacted ex vivo with a prime editing composition or pharmaceutical composition thereof and are re-introduced into the subject.

In some embodiments, cells are contacted ex vivo with one or more components of a prime editing composition. In some embodiments, the ex vivo-contacted cells are introduced into the subject, and the subject is administered in vivo with one or more components of a prime editing composition. For example, in some embodiments, cells are contacted ex vivo with a prime editor and introduced into a subject. In some embodiments, the subject is then administered with a PEgRNA and/or a ngRNA, or a polynucleotide encoding the PEgRNA and/or the ngRNA.

In some embodiments, cells contacted with the prime editing composition are determined for incorporation of the one or more intended nucleotide edits in the genome before re-introduction into the subject. In some embodiments, the cells are enriched for incorporation of the one or more intended nucleotide edits in the genome before re-introduction into the subject. In some embodiments, the edited cells are primary cells. In some embodiments, the edited cells are progenitor cells. In some embodiments, the edited cells are stem cells. In some embodiments, the edited cells are hepatocytes. In some embodiments, the edited cells are primary human cells. In some embodiments, the edited cells are human progenitor cells. In some embodiments, the edited cells are human stem cells.

In some embodiments, the edited cells are induced human pluripotent stem cells (iPSCs). In some embodiments, the edited cells are lung progenitor cells. In some embodiments, the edited cells are bronchioalveolar stem cells. In some embodiments, the edited cells are human bronchioalveolar stem cells. In some embodiments, the edited cells are human lung progenitor cells. In some embodiments, the edited cells are basal cells. In some embodiments, the edited cells are club cells. In some embodiments, the edited cells are ciliated cells. In some embodiments, the edited cells are club cells. In some embodiments, the edited cells are ionocytes, e.g., pulmonary human ionocytes. In some embodiments, the edited cells are human basal cells. In some embodiments, the cell is a human basal cell from the respiratory epithelium. In some embodiments, the edited cells are human club cells. In some embodiments, the edited cells are human ciliated cells. In some embodiments, the edited cells are club cells. In some embodiments, the edited cells are human ionocytes, e.g., pulmonary human ionocytes. In some embodiments, the edited cells are epithelial cells. In some embodiments, the edited cells are airway epithelial cells. In some embodiments, the edited cells are human airway epithelial cells. In some embodiments, the edited cells are bronchial epithelial cells. In some embodiments, the edited cells are human bronchial epithelial cells. In some embodiments, the edited cells are human bronchial epithelial cells induced from iPSCs. In some embodiments, the edited cells are pancreatic epithelial cells. In some embodiments, the edited cells are pancreatic ductal epithelia cells. In some embodiments, the edited cells are pancreatic acinar cells. In some embodiments, the edited cells are human pancreatic epithelial cells. In some embodiments, the edited cells are human pancreatic ductal epithelia cells. In some embodiments, the edited cells are human pancreatic acinar cells. In some embodiments, the edited cells are hepatocytes. In some embodiments, the edited cells are human hepatocytes. In some embodiments, the edited cells are kidney epithelial cells. In some embodiments, the edited cells are intestine epithelial cells. In some embodiments, the edited cells are human kidney epithelial cells. In some embodiments, the edited cells are human intestine epithelial cells. In some embodiments, the edited cells are tuft cells. In some embodiments, the edited cells are neuroendocrine cells. In some embodiments, the edited cells are goblet cells. In some embodiments, the edited cells are reproductive tissue epithelial cells. In some embodiments, the edited cells are sperm canal epithelial cells. In some embodiments, the edited cells are human tuft cells. In some embodiments, the edited cells are human neuroendocrine cells. In some embodiments, the edited cells are human goblet cells. In some embodiments, the edited cells are human reproductive tissue epithelial cells. In some embodiments, the edited cells are human sperm canal epithelial cells. In some embodiments, the edited cells are an ex vivo cells. In some embodiments, the edited cells are an ex vivo cells obtained from a human subject. In some embodiments, the edited cells are in a subject, e.g., a human subject.

The prime editing composition or components thereof may be introduced into a cell by any delivery approaches as described herein, including LNP administration, RNP administration, electroporation, nucleofection, transfection, viral transduction, microinjection, cell membrane disruption and diffusion, or any other approach known in the art.

The cells edited with prime editing can be introduced into the subject by any route known in the art. In some embodiments, the edited cells are administered to a subject by direct infusion. In some embodiments, the edited cells are administered to a subject by intravenous infusion. In some embodiments, the edited cells are administered to a subject as implants.

The pharmaceutical compositions, prime editing compositions, and cells, as described herein, can be administered in effective amounts. In some embodiments, the effective amount depends upon the mode of administration. In some embodiments, the effective amount depends upon the stage of the condition, the age and physical condition of the subject, the nature of concurrent therapy, if any, and like factors well-known to the medical practitioner.

The specific dose administered can be a uniform dose for each subject. Alternatively, a subject's dose can be tailored to the approximate body weight of the subject. Other factors in determining the appropriate dosage can include the disease or condition to be treated or prevented, the severity of the disease, the route of administration, and the age, sex and medical condition of the patient.

In embodiments wherein components of a prime editing composition are administered sequentially, the time between sequential administration can be at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days.

In some embodiments, a method of monitoring treatment progress is provided. In some embodiments, the method includes the step of determining a level of diagnostic marker, for example, correction of a mutation in CFTR gene, or diagnostic measurement associated with Cystic Fibrosis, (e.g., chloride efflux assay) in a subject suffering from Cystic Fibrosis symptoms and has been administered an effective amount of a prime editing composition described herein. The level of the diagnostic marker determined in the method can be compared to known levels of the marker in either healthy normal controls or in other afflicted subjects to establish the subject's disease status.

### Delivery

Prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, a PEgRNA can be delivered directly as an RNA or as a DNA encoding the PEgRNA.

In some embodiments, a prime editing composition component is encoded by a polynucleotide, a vector, or a construct. In some embodiments, a prime editor polypeptide, a PEgRNA and/or a ngRNA is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA polymerase domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PEgRNA and/or a ngRNA. In some embodiments, the polypeptide encodes two or more components of a prime editing composition, for example, a prime editor fusion protein and a PEgRNA.

In some embodiments, the polynucleotide encoding one or more prime editing composition components is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, H1 promoter).

In some embodiments, the polynucleotide encoding one or more prime editing composition components is a part of, or is encoded by, a vector. In some embodiments, the vector is a viral vector. In some embodiments, the vector is a non-viral vector.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA, e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, a mRNA that encodes a prime editor polypeptide is generated using in vitro transcription. Guide polynucleotides (e.g., PEgRNA or ngRNA) can also be transcribed using in vitro transcription from a cassette containing a T7 promoter, followed by the sequence “GG”, and guide polynucleotide sequence. In some embodiments, the prime editor encoding mRNA, PEgRNA, and/or ngRNA are synthesized in vitro using an RNA polymerase enzyme (e.g., T7 polymerase, T3 polymerase, SP6 polymerase, etc.). Once synthesized, the RNA can directly contact a target CFTR gene or can be introduced into a cell using any suitable technique for introducing nucleic acids into cells (e.g., microinjection, electroporation, transfection). In some embodiments, the prime editor-coding sequences, the PEgRNAs, and/or the ngRNAs are modified to include one or more modified nucleoside e.g. using pseudo-U or 5-Methyl-C.

Methods of non-viral delivery of nucleic acids can include lipofection, electroporation, nucleofection, microinjection, biolistics, virosomes, liposomes, immunoliposomes, polycation or lipid:nucleic acid conjugates, nanoparticles, cell penetrating peptides and associated conjugated molecules and chemistry, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid:nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered after delivery (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), Simian Immuno deficiency virus (SIV), human immuno deficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector.

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and .psi.2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. In some embodiment, the polynucleotides are a DNA polynucleotide. In some embodiment, the polynucleotides are an RNA polynucleotide; e.g., an mRNA polynucleotide.

In some embodiments, the AAV vector is selected for tropism to a particular cell, tissue, organism. In some embodiments, the AAV vector is pseudotyped, e.g., AAV5/8. In some embodiments, polynucleotides encoding one or more prime editing composition components are packaged in a first AAV and a second AAV. In some embodiments, the polynucleotides encoding one or more prime editing composition components are packaged in a first rAAV and a second rAAV.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g., a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. In some embodiments, intein-N may be fused to the N-terminal portion of a first domain described herein, and and intein-C may be fused to the C-terminal portion of a second domain described herein for the joining of the N-terminal portion to the C-terminal portion, thereby joining the first and second domains. In some embodiments, the first and second domains are each independently chosen from a DNA binding domain or a DNA polymerase domain. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the full-length prime editor fusion protein is reassembled upon co-infection of the same cell by both dual AAV vectors, expression of both halves of the prime editor fusion protein, and self-excision of the inteins.

In some embodiments, the in vivo use of dual AAV vectors results in the expression of full-length full-length prime editor fusion proteins. In some embodiments, the use of the dual AAV vector platform allows viable delivery of transgenes of greater than about 4.5, 4.6, 4.7, 4.8, 4.9, or 5.0 kb in size.

In some embodiments, an intein is inserted at a splice site within a Cas protein. In some embodiments, insertion of an intein disrupts a Cas activity. As used herein, “intein” refers to a self-splicing protein intron (e.g., peptide), e.g., which ligates flanking N-terminal and C-terminal exteins (e.g., fragments to be joined). In some embodiments, an intein may comprise a polypeptide that is able to excise itself and join exteins with a peptide bond (e.g., protein splicing). In some embodiments, an intein of a precursor gene comes from two genes (e.g., split intein). In some embodiments, an intein may be a synthetic intein. Non-limiting examples of intein pairs that may be used in accordance with the present disclosure include: dnaE-n and dnaE-c. a 4-hydroxytamoxifen (4-HT)-responsive intein, an iCas molecule, a Ssp DnaX intein, Ter DnaE3 intein, Ter ThyX intein, Rma DnaB intein, Cfa DnaE intein, Ssp GyrB intein, and Rma DnaB intein. In some embodiments, intein fragments may be fused to the N terminal and C-terminal portion of a split Cas protein respectively for joining the fragments of split Cas9.

In some embodiments, the split Cas9 system may be used in general to bypass the packing limit of the viral delivery vehicles. In some embodiments, a split Cas9 may be a Type II CRISPR system Cas9. In some embodiments, a first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein. In some embodiments, the first portion of the Cas9 protein is the N-terminal fragment of the Cas9 protein and the second portion of the Cas9 protein is the C-terminal fragment of the Cas9 protein. In some embodiments, a split site may be selected which are surface exposed due to the sterical need for protein splicing.

In some embodiments, a Cas protein may be split into two fragments at any C, T, A, or S. In some embodiments, a Cas9 may be intein split at residues 203-204, 280-292, 292-364, 311-325, 417-438, 445-483, 468-469, 481-502, 513-520, 522-530, 565-637, 696-707, 713-714, 795-804, 803-810, 878-887, and 1153-1154. In some embodiments, protein is divided into two fragments at SpCas9 T310, T313, A456, S469, or C574. In some embodiments, split Cas9 fragments across different split pairs yield combinations that provided the complete polypeptide sequence activate gene expression even when fragments are partially redundant. In some embodiments, a functional Cas9 protein may be reconstituted from two inactive split-Cas9 peptides in the presence of gRNA by using a split-intein protein splicing strategy. In some embodiment, the split Cas9 fragments are fused to either a N-terminal intein fragment or a C-terminal intein fragment, which can associate with each other and catalytically splice the two split Cas9 fragments into a functional reconstituted Cas9 protein. In some embodiments, a split-Cas9 can be packaged into self-complementary AAV. In some embodiments, a split-Cas9 comprises a 2.5 kb and a 2.2 kb fragment of S. pyogenes Cas9 coding sequences.

In some embodiments, a split-Cas9 architecture reduces the length and/or size of the coding sequences of a viral vector, e.g., AAV.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a including peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 346). As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine (SEQ ID NO: 21633), and octa-arginine (SEQ ID NO: 21634). The nona-arginine (R9) sequence (SEQ ID NO: 21633) can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, prime editor polypeptide components and PEgRNA/ngRNA are introduced to a target cell by nanoparticles. In some embodiments, the prime editor polypeptide components and the PEgRNA and/or ngRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., an epithelial cell, in an organic nanoparticle, e.g. a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids used to produce LNPs are provided in Table 4 below.

In some embodiments, components of a prime editing composition form a complex prior to delivery to a target cell. For example, a prime editor fusion protein, a PEgRNA, and/or a ngRNA can form a complex prior to delivery to the target cell. In some embodiments, a prime editing polypeptide (e.g. a prime editor fusion protein) and a guide polynucleotide (e.g. a PEgRNA or ngRNA) form a ribonucleoprotein (RNP) for delivery to a target cell. In some embodiments, the RNP comprises a prime editor fusion protein complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary lipids for use in nanoparticle formulations and/or gene transfer are shown in Table 4 below.

TABLE 4
Exemplary lipids for nanoparticle formulation or gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N41-(2,3-Dioleyloxy)prophyliN,N,N-trimethylammonium | DOTMA | Cationic |
| chloride | | |
| 1,2-Dioleoyloxy-3-trimethylammonium-propane | DOGS | Cationic |
| Dioctadecylamidoglycylspermine | | |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl omithinate | LHON | Cationic |
| 1-(2,3-Dioleoyloxypropyl)-2,4,6-trimethylpyridinium | 2Oc | Cationic |
| 2,3-Dioleyloxy-N-P(spenninecarboxamido-ethylJ-N,Ndimethyl- | DOSPA | Cationic |
| 1-propanatninium trifluoroacetate | | |
| 1,2-Dioleyl-3-trimethylamtnonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyloxymethyloxy) | CLIP-6 | Cationic |
| ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spenmine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-Bis(3-amino-propyl)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Exemplary polymers for use in nanoparticle formulations and/or gene transfer are shown in Table 5 below.

TABLE 5
Exemplary lipids for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethy1-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for polynucleotides encoding prime editing composition components are shown in Table 6 below.

TABLE 6
Exemplary polynucleotide delivery methods

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium phosphate | | | | |
| | transfection) | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic acids |
| | | | | what is | and Proteins |
| | | | | delivered | |
| | Polymeric | YES | Transient | NO | Nucleic Acids |
| | Nanoparticles | | | | |
| Biological | Attenuated Bacteria | YES | Transient | NO | Nucleic Acids |
| Non-Viral | Engineered | YES | Transient | NO | Nucleic Acids |
| Delivery | Bacteriophages | | | | |
| Vehicles | Mammalian Virus- | YES | Transient | NO | Nucleic Acids |
| | like Particles | | | | |
| | Biological | YES | Transient | NO | Nucleic Acids |
| | liposomes: | | | | |
| | Erythrocyte Ghosts | | | | |
| | and Exosomes | | | | |

The prime editing compositions of the disclosure, whether introduced as polynucleotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g., two different polynucleotide constructs are provided to the cell (e.g., different components of the same prim editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g. two different polynucleotide constructs are administered to the subject (e.g., different components of the same prim editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

### EXAMPLES

### Example 1—Screening of PEgRNA for Editing of a Mutation Associated with Cystic Fibrosis

The following examples are provided for illustrative purposes only and are not intended to limit the scope of the claims provided herein.

PEgRNA assembly: PEgRNAs are cloned as follows: double-stranded linear DNA fragments encoding PEgRNA and flanking U6 expression plasmid homology sequences are individually Gibson-cloned into U6 expression plasmids. Alternatively, for each PEgRNA, separate oligos encoding a protospacer, a gRNA scaffold, and PEgRNA extension (PBS and RTT) are ligated, and then cloned into a U6 expression plasmid as described in Anzalone et al., Nature. 2019 December; 576(7785):149-157. Bacterial colonies carrying sequence-verified plasmids are propagated in LB or TB. Plasmid DNA is purified by minipreps for mammalian transfection.

Chemically synthesized PEgRNAs are also used (Integrated DNA Technologies, following the vendor's standard procedure).

HEK cell culture and transfection: PEgRNAs each harboring a mutation corresponding to one of F508del, G542*, S549N, G551D, R553* mutations in the CFTR polypeptide are used for installation in wild type HEK293T cells, using installation frequency as a proxy for editing efficiency. HEK293T cells are propagated in DMEM with 10% FBS. Prior to transfection, cells are seeded in 96-well plates and then transfected with Lipofectamine 2000 according to the manufacturer's directions with plasmids encoding PEgRNAs as described above and a prime editor (PE) fusion, or transfected with Lipofectamine MessengerMAX™ with mRNA encoding the PE and synthetic PEgRNAs. Three days after transfection, gDNA is harvested in lysis buffer for high throughput sequencing and sequenced using Miseq. PEgRNA sequences are provided in Table 7.

Lentiviral production and cell line generation and CFTR mutation correction: DNA sequences each containing one of F508del, G542*, S549N, G551D, R553* amino acid mutations respectively with flanking sequences from the CFTR gene on each side, and an EF11a short promoter driven Puromycin selection cassette, are cloned in lentiviral transfer plasmids. HEK 293T cells are transiently transfected with the transfer plasmids and packaging plasmids containing VSV glycoprotein and lentiviral gag/pol coding sequences. After transfection, lentiviral particles are harvested from the cell media and concentrated. HEK 293T cells are transduced using serial dilutions of the lentiviral particles. Cells generated at a dilution of MOI<1, as determined by survival following puromycin, are selected for expansion. PEgRNAs each designed to correct one of the F508del, G542*, S549N, R553*, and G551D mutations are also transfected in each cell line that harbors the lentivirus that has the corresponding mutation. Three days after transfection, gDNA is harvested in lysis buffer for high throughput sequencing and sequenced using Miseq. Prime editing efficiency of each PEgRNA in each of the resulting HEK293T cell lines is examined using Miseq. PEgRNA and ngRNA sequences are provided in Table 8.

Prime editing of CFTR in human cells: PEgRNAs screened from the lentiviral assay as described above are tested in human fibroblasts, lymphoblasts, and iPSCs, respectively. Human fibroblasts lymphoblasts, and iPSCs carrying CFTR mutations are transfected with RNA encoding the PE2 protein and the PEgRNA as tested above. Genomic DNA is harvested after a 1-week incubation, and editing efficiency is examined using Miseq.

Chloride efflux assay: N-[ethoxycarbonylmethyl]-6-methoxy-quinolinium bromide (MQAE), a chloride sensitive fluorescent dye, is used to assess chloride flux in plated human intestinal epithelial cells. Cells are grown to confluence directly on coverslips. Then, cells are placed in C1-containing solution (135 mM NaCl, 5 mM KCl, 1 mM CaCl2), 1.2 mM MgSO4, 2 mM NaH2PO4, 2 mM HEPES, and 10 mM glucose), then moved to a chloride free solution (135 mM NaCyclamate, 3 mM KGluconate, 0.5 mM CaCyclamate, 1.2 mM MgSO4, 2 mM KH2PO4, 2 mM HEPES, 10 mM glucose). Chloride flux is assessed in solution with forskolin (10 μM) and IBMX (100 μM) added. MQAE fluorescence is examined on an Olympus IX-71 inverted microscope, with MQAE excited at 354 nm and fluorescence measured at 460 nm every 5 s. Fluorescence is measured on a cell-by-cell basis, with 30 to 100 cells catalogued per slide. The rate of change in MQAE fluorescence (arbitrary fluorescence units AFU/time) is graphed, and AFU/min is compared between groups.

Lengthy table referenced here
US20240301444A1-20240912-T00001
Please refer to the end of the specification for access instructions.

Lengthy table referenced here
US20240301444A1-20240912-T00002
Please refer to the end of the specification for access instructions.

Lengthy table referenced here
US20240301444A1-20240912-T00003
Please refer to the end of the specification for access instructions.

Lengthy table referenced here
US20240301444A1-20240912-T00004
Please refer to the end of the specification for access instructions.

Lengthy table referenced here
US20240301444A1-20240912-T00005
Please refer to the end of the specification for access instructions.

Lengthy table referenced here
US20240301444A1-20240912-T00006
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240301444A1). An electronic copy of the table will also be available from the USPTO upon request and payment of the fee set forth in 37 CFR 1.19(b)(3).

1. A prime editing guide RNA (PEgRNA) comprising:
a spacer that is complementary to a search target sequence on a first strand of a CFTR gene,
an editing template that comprises a region of complementarity to an editing target sequence on a second strand of the CFTR gene, and
a gRNA core that associates with a prime editor comprising a DNA binding domain and a DNA polymerase domain,
wherein the first strand and the second strand are complementary to each other, and
a) wherein the editing target sequence is in an exon selected from the group consisting of: exon 1, exon 2, exon 3, exon 4, exon 5, exon 6, exon 7, exon 8, exon 9, exon 10, exon 12, exon 13, exon 14, exon 15, exon 16, exon 17, exon 18, exon 19, exon 20, exon 21, exon 22, exon 23, exon 24, exon 25, exon 26, and exon 27 of the CFTR gene;
b) wherein if the editing target sequence is in exon 11, then the editing target sequence does not comprise a CTT deletion at positions chr7:117559591-117559594 as compared to a wild type CFTR gene;
c) wherein the editing target sequence is between positions 117480095-117548823 and positions 117587739-117665564 of human chromosome 7: or
d) wherein the editing target sequence comprises a mutation associated with cystic fibrosis, and wherein the mutation is not the CTT deletion at positions chr7:117559591-117559594 as compared to the wild type CFTR gene.

2.-4. (canceled)

5. The PEgRNA of claim 1, wherein the PEgRNA comprises a primer binding site sequence (PBS) at least partially complementary to the spacer.

6. The PEgRNA of claim 1, wherein the gRNA core is between the spacer and the editing template.

7. The PEgRNA of claim 1, wherein the editing template comprises an intended nucleotide edit compared to the CFTR gene.

8. The PEgRNA of claim 7, wherein the intended nucleotide edit results in the CFTR gene encoding wild-type protein sequence.

9. (canceled)

10. The PEgRNA of claim 7, wherein the search target sequence is complementary to a protospacer sequence in the CFTR gene, and wherein the protospacer sequence is adjacent to a protospacer adjacent motif (PAM) in the second strand of the CFTR gene.

11.-12. (canceled)

13. The PEgRNA of claim 5, wherein the PBS is about 8 to 16 base pairs in length.

14. The PEgRNA of claim 1, wherein the editing template is about 4 to 30 base pairs in length.

15. The PEgRNA of claim 14, wherein the editing template is about 10 to 30 base pairs in length.

16. The PEgRNA of claim 10, wherein the intended nucleotide edit is configured for incorporation at a position about 0 to 27 base pairs downstream of the 5′ end of the PAM.

17. The PEgRNA of claim 7, wherein the intended nucleotide edit comprises a single nucleotide substitution, an insertion, or a deletion in the CFTR gene.

18.-33. (canceled)

34. The PEgRNA of claim 1, wherein the editing target sequence comprises a mutation that encodes an amino acid substitution selected from the group consisting of: G85E, R117H, R334W, R347P, R347H, A445E, G542*, S549N, G551D, R553*, K684fs, D1152H, R1158*, R1162*, K1177R, W1282*, and N1303K as compared to a wild type CFTR protein as set forth in SEQ ID NO: 19, wherein * refers to a nonsense mutation and fs refers to a frameshift mutation.

35. A PEgRNA system comprising:
(i) a PEgRNA comprising:
a spacer that is complementary to a search target sequence on a first strand of a CFTR gene,
an editing template that comprises a region of complementarity to an editing target sequence on a second strand of the CFTR gene, and
a gRNA core that associates with a prime editor comprising a DNA binding domain and a DNA polymerase domain,
wherein the first strand and the second strand are complementary to each other, and
a) wherein the editing target sequence is in an exon selected from the group consisting of: exon 1, exon 2, exon 3, exon 4, exon 5, exon 6, exon 7, exon 8, exon 9, exon 10, exon 12, exon 13, exon 14, exon 15, exon 16, exon 17, exon 18, exon 19, exon 20, exon 21, exon 22, exon 23, exon 24, exon 25, exon 26, and exon 27 of the CFTR gene;
b) wherein if the editing target sequence is in exon 11 then the editing target sequence does not comprise a CTT deletion at positions chr7:117559591-117559594 as compared to a wild type CFTR gene;
c) wherein the editing target sequence is between positions 117480095-117548823 and positions 117587739-117665564 of human chromosome 7; or
d) wherein the editing target sequence comprises a mutation associated with cystic fibrosis, and wherein the mutation is not a CTT deletion at positions chr7:117559591-117559594 as compared to a wild type CFTR gene; and
(ii) a nick guide RNA (ngRNA) comprising an ng spacer that is complementary to a second search target sequence in the CFTR gene.

36. The PEgRNA system of claim 35, wherein the second search target sequence is on the second strand of the CFTR gene.

37. A prime editing guide RNA (PEgRNA) comprising:
a spacer comprising a sequence selected from the group consisting of SEQ ID NOs: 678-682, 691-709, and 712-728,
an editing template that comprises a region of complementarity to an editing target sequence on an edit strand of a CFTR gene, and
a gRNA core that associates with a prime editor comprising a DNA binding domain and a DNA polymerase domain,
wherein a first strand of the CFTR gene and a second strand of the CFTR gene are complementary to each other, and wherein the editing target sequence comprises a CTT deletion at positions chr7:117559591-117559594 as compared to a wild type CFTR gene.

38. The PEgRNA of claim 37, wherein the PEgRNA comprises a primer binding site sequence (PBS) that is at least partially complementary to the spacer.

39. The PEgRNA of claim 38, wherein the PBS comprises a sequence selected from the group consisting of SEQ Identifiers: B1377-B1403, B1413-B1520, and B1530-B1565.

40. The PEgRNA of claim 38, wherein the PBS comprises a sequence selected from the group consisting of SEQ ID NOs: 2284-2304, 2312-2395, and 2403-2430.

41. The PEgRNA of claim 38, wherein the PBS comprises a sequence selected from the group consisting of sequences: ATAATCCA, ATAATCCAG, CATAATCC, CATAATCCA, TGGTGCCA, TGGTGCCAG, TATTTTCT, TATTTTCTT, ATATTTTC, ATATTTTCT, GATATTTT, GATATTTTC, ATGAATAT, ATGAATATA, GCGTCATC, GCGTCATCA, TAATCCAG, TAATCCAGG, GTGTTTCC, GTGTTTCCT, TGAATATA, TGAATATAG, GATGAATA, GATGAATAT, ATGGTGCC, ATGGTGCCA, TGTTTCCT, TGTTTCCTA, TCCAGGAA, TCCAGGAAA, ATGATATT, ATGATATTT, TGATATTT, TGATATTTT, TCAAAGCA, TCAAAGCAT, GATGATAT, and GATGATATT.

42. The PEgRNA of claim 37, wherein the editing template comprises a sequence selected from the group consisting of SEQ ID NOs: 8618-8990 and 9002-9440.

43.-82. (canceled)
